SEQUENCE LISTING (445 sequences)

SEQ ID NO: 1 - protein, 735 aa, Adeno-associated virus 2 [sequence omitted]

SEQ ID NO: 2 - DNA, 2208 bp, Adeno-associated virus 2
  source 1..2208: mol_type = genomic DNA
atggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggctccgggaaaaaagaggccggtagagcactctcctgtggagccagactcctcctcgggaaccggaaaggcgggccagcagcctgcaagaaaaagattgaattttggtcagactggagacgcagactcagtacctgacccccagcctctcggacagccaccagcagccccctctggtctgggaactaatacgatggctacaggcagtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtattacttgagcagaacaaacactccaagtggaaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcaacagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagtacacttccaactacaacaagtctgttaatgtggactttactgtggacactaatggcgtgtattcagagcctcgccccattggcaccagatacctgactcgtaatctgtaa

SEQ ID NO: 3 - protein, 724 aa, Adeno-associated virus 5
MSFVDHPPDWLEEVGEGLREFLGLEAGPPKPKPNQQHQDQARGLVLPGYNYLGPGNGLDRGEPVNRADEVAREHDISYNEQLEAGDNPYLKYNHADAEFQEKLADDTSFGGNLGKAVFQAKKRVLEPFGLVEEGAKTAPTGKRIDDHFPKRKKARTEEDSKPSTSSDAEAGPSGSQQLQIPAQPASSLGADTMSAGGGGPLGDNNQGADGVGNASGDWHCDSTWMGDRVVTKSTRTWVLPSYNNHQYREIKSGSVDGSNANAYFGYSTPWGYFDFNRFHSHWSPRDWQRLINNYWGFRPRSLRVKIFNIQVKEVTVQDSTTTIANNLTSTVQVFTDDDYQLPYVVGNGTEGCLPAFPPQVFTLPQYGYATLNRDNTENPTERSSFFCLEYFPSKMLRTGNNFEFTYNFEEVPFHSSFAPSQNLFKLANPLVDQYLYRFVSTNNTGGVQFNKNLAGRYANTYKNWFPGPMGRTQGWNLGSGVNRASVSAFATTNRMELEGASYQVPPQPNGMTNNLQGSNTYALENTMIFNSQPANPGTTATYLEGNMLITSESETQPVNRVAYNVGGQMATNNQSSTTAPATGTYNLQEIVPGSVWMERDVYLQGPIWAKIPETGAHFHPSPAMGGFGLKHPPPMMLIKNTPVPGNITSFSDVPVSSFITQYSTGQVTVEMEWELKKENSKRWNPEIQYTNNYNDPQFVDFAPDSTGEYRTTRPIGTRYLTRPL

SEQ ID NO: 4 - DNA, 2175 bp, Adeno-associated virus 5
  source 1..2175: mol_type = genomic DNA
atgtcttttgttgatcaccctccagattggttggaagaagttggtgaaggtcttcgcgagtttttgggccttgaagcgggcccaccgaaaccaaaacccaatcagcagcatcaagatcaagcccgtggtcttgtgctgcctggttataactatctcggacccggaaacgggctcgatcgaggagagcctgtcaacagggcagacgaggtcgcgcgagagcacgacatctcgtacaacgagcagcttgaggcgggagacaacccctacctcaagtacaaccacgcggacgccgagtttcaggagaagctcgccgacgacacatccttcgggggaaacctcggaaaggcagtctttcaggccaagaaaagggttctcgaaccttttggcctggttgaagagggtgctaagacggcccctaccggaaagcggatagacgaccactttccaaaaagaaagaaggctcggaccgaagaggactccaagccttccacctcgtcagacgccgaagctggacccagcggatcccagcagctgcaaatcccagcccaaccagcctcaagtttgggagctgatacaatgtctgcgggaggtggcggcccattgggcgacaataaccaaggtgccgatggagtgggcaatgcctcgggagattggcattgcgattccacgtggatgggggacagagtcgtcaccaagtccacccgaacctgggtgctgcccagctacaacaaccaccagtaccgagagatcaaaagcggctccgtcgacggaagcaacgccaacgcctactttggatacagcaccccctgggggtactttgactttaaccgcttccacagccactggagcccccgagactggcaaagactcatcaacaactactggggcttcagaccccggtccctcagagtcaaaatcttcaacattcaagtcaaagaggtcacggtgcaggactccaccaccaccatcgccaacaacctcacctccaccgtccaagtgtttacggacgacgactaccagctgccctacgtcgtcggcaacgggaccgagggatgcctgccggccttccctccgcaggtctttacgctgccgcagtacggttacgcgacgctgaaccgcgacaacacagaaaatcccaccgagaggagcagcttcttctgcctagagtactttcccagcaagatgctgagaacgggcaacaactttgagtttacctacaactttgaggaggtgcccttccactccagcttcgctcccagtcagaacctgttcaagctggccaacccgctggtggaccagtacttgtaccgcttcgtgagcacaaataacactggcggagtccagttcaacaagaacctggccgggagatacgccaacacctacaaaaactggttcccggggcccatgggccgaacccagggctggaacctgggctccggggtcaaccgcgccagtgtcagcgccttcgccacgaccaataggatggagctcgagggcgcgagttaccaggtgcccccgcagccgaacggcatgaccaacaacctccagggcagcaacacctatgccctggagaacactatgatcttcaacagccagccggcgaacccgggcaccaccgccacgtacctcgagggcaacatgctcatcaccagcgagagcgagacgcagccggtgaaccgcgtggcgtacaacgtcggcgggcagatggccaccaacaaccagagctccaccactgcccccgcgaccggcacgtacaacctccaggaaatcgtgcccggcagcgtgtggatggagagggacgtgtacctccaaggacccatctgggccaagatcccagagacgggggcgcactttcacccctctccggccatgggcggattcggactcaaacacccaccgcccatgatgctcatcaagaacacgcctgtgcccggaaatatcaccagcttctcggacgtgcccgtcagcagcttcatcacccagtacagcaccgggcaggtcaccgtggagatggagtgggagctcaagaaggaaaactccaagaggtggaacccagagatccagtacacaaacaactacaacgacccccagtttgtggactttgccccggacagcaccggggaatacagaaccaccagacctatcggaacccgataccttacccgacccctttaa

SEQ ID NO: 5 - protein, 738 aa, Adeno-associated virus 8
MAADGYLPDWLEDNLSEGIREWWALKPGAPKPKANQQKQDDGRGLVLPGYKYLGPFNGLDKGEPVNAADAAALEHDKAYDQQLQAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGDSESVPDPQPLGEPPAAPSGVGPNTMAAGGGAPMADNNEGADGVGSSSGNWHCDSTWLGDRVITTSTRTWALPTYNNHLYKQISNGTSGGATNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLSRTQTTGGTANTQTLGFSQGGPNTMANQAKNWLPGPCYRQQRVSTTTGQNNNSNFAWTAGTKYHLNGRNSLANPGIAMATHKDDEERFFPSNGILIFGKQNAARDNADYSDVMLTSEEEIKTTNPVATEEYGIVADNLQQQNTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL

SEQ ID NO: 6 - DNA, 2217 bp, Adeno-associated virus 8
  source 1..2217: mol_type = genomic DNA
atggctgccgatggttatcttccagattggctcgaggacaacctctctgagggcattcgcgagtggtgggcgctgaaacctggagccccgaagcccaaagccaaccagcaaaagcaggacgacggccggggtctggtgcttcctggctacaagtacctcggacccttcaacggactcgacaagggggagcccgtcaacgcggcggacgcagcggccctcgagcacgacaaggcctacgaccagcagctgcaggcgggtgacaatccgtacctgcggtataaccacgccgacgccgagtttcaggagcgtctgcaagaagatacgtcttttgggggcaacctcgggcgagcagtcttccaggccaagaagcgggttctcgaacctctcggtctggttgaggaaggcgctaagacggctcctggaaagaagagaccggtagagccatcaccccagcgttctccagactcctctacgggcatcggcaagaaaggccaacagcccgccagaaaaagactcaattttggtcagactggcgactcagagtcagttccagaccctcaacctctcggagaacctccagcagcgccctctggtgtgggacctaatacaatggctgcaggcggtggcgcaccaatggcagacaataacgaaggcgccgacggagtgggtagttcctcgggaaattggcattgcgattccacatggctgggcgacagagtcatcaccaccagcacccgaacctgggccctgcccacctacaacaaccacctctacaagcaaatctccaacgggacatcgggaggagccaccaacgacaacacctacttcggctacagcaccccctgggggtattttgactttaacagattccactgccacttttcaccacgtgactggcagcgactcatcaacaacaactggggattccggcccaagagactcagcttcaagctcttcaacatccaggtcaaggaggtcacgcagaatgaaggcaccaagaccatcgccaataacctcaccagcaccatccaggtgtttacggactcggagtaccagctgccgtacgttctcggctctgcccaccagggctgcctgcctccgttcccggcggacgtgttcatgattccccagtacggctacctaacactcaacaacggtagtcaggccgtgggacgctcctccttctactgcctggaatactttccttcgcagatgctgagaaccggcaacaacttccagtttacttacaccttcgaggacgtgcctttccacagcagctacgcccacagccagagcttggaccggctgatgaatcctctgattgaccagtacctgtactacttgtctcggactcaaacaacaggaggcacggcaaatacgcagactctgggcttcagccaaggtgggcctaatacaatggccaatcaggcaaagaactggctgccaggaccctgttaccgccaacaacgcgtctcaacgacaaccgggcaaaacaacaatagcaactttgcctggactgctgggaccaaataccatctgaatggaagaaattcattggctaatcctggcatcgctatggcaacacacaaagacgacgaggagcgtttttttcccagtaacgggatcctgatttttggcaaacaaaatgctgccagagacaatgcggattacagcgatgtcatgctcaccagcgaggaagaaatcaaaaccactaaccctgtggctacagaggaatacggtatcgtggcagataacttgcagcagcaaaacacggctcctcaaattggaactgtcaacagccagggggccttacccggtatggtctggcagaaccgggacgtgtacctgcagggtcccatctgggccaagattcctcacacggacggcaacttccacccgtctccgctgatgggcggctttggcctgaaacatcctccgcctcagatcctgatcaagaacacgcctgtacctgcggatcctccgaccaccttcaaccagtcaaagctgaactctttcatcacgcaatacagcaccggacaggtcagcgtggaaattgaatgggagctgcagaaggaaaacagcaagcgctggaaccccgagatccagtacacctccaactactacaaatctacaagtgtggactttgctgttaatacagaaggcgtgtactctgaaccccgccccattggcacccgttacctcacccgtaatctgtaa

SEQ ID NO: 7 - protein, 736 aa, Adeno-associated vrius 9
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 8 - DNA, 2211 bp, Adeno-associated vrius 9
  source 1..2211: mol_type = genomic DNA
atggctgccgatggttatcttccagattggctcgaggacaaccttagtgaaggtattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcccaagcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaa

SEQ ID NO: 9 - protein, 738 aa, Adeno-associated virus
MAADGYLPDWLEDNLSEGIREWWDLKPGAPKPKANQQKQDNGRGLVLPGYKYLGPFNGLDKGEPVNAADAAALEHDKAYDQQLQAGDNPYLRYNHADAEFQERLQEDTSFGGNLGRAVFQAKKRVLEPLGLVESPVKTAPGKKRPVEPSPQRSPDSSTGIGKKGQQPAKKRLNFGQTGDSESVPDPQPIGEPPAGPSGLGSGTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLNFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFEFSYNFEDVPFHSSYAHSQSLDRLMNPLIDQYLYYLSRTQSTGGTAGTQQLLFSQAGPNNMSAQAKNWLPGPCYRQQRVSTTLSQNNNSNFAWTGATKYHLNGRDSLVNPGVAMATHKDDEERFFPSSGVLMFGKQGAGKDNVDYSSVMLTSEEEIKTTNPVATEQYGVVADNLQQQNAAPIVGAVNSQGALPGMVWQNRDVYLQGPIWAKIPHTDGNFHPSPLMGGFGLKHPPPQILIKNTPVPADPPTTFNQAKLASFITQYSTGQVSVEIEWELQKENSKRWNPEIQYTSNYYKSTNVDFAVNTEGTYSEPRPIGTRYLTRNL

SEQ ID NO: 10 - DNA, 2217 bp, Adeno-associated virus
  source 1..2217: mol_type = genomic DNA
atggctgccgatggttatcttccagattggctcgaggacaacctctctgagggcattcgcgagtggtgggacctgaaacctggagccccgaaacccaaagccaaccagcaaaagcaggacaacggccggggtctggtgcttcctggctacaagtacctcggacccttcaacggactcgacaagggggagcccgtcaacgcggcggacgcagcggccctcgagcacgacaaggcctacgaccagcagctccaagcgggtgacaatccgtacctgcggtataatcacgccgacgccgagtttcaggagcgtctgcaagaagatacgtcttttgggggcaacctcgggcgcgcagtcttccaggccaaaaagcgggttctcgaacctctgggcctggttgaatcgccggttaagacggctcctggaaagaagaggccggtagagccatcaccccagcgctctccagactcctctacgggcatcggcaagaaaggccagcagcccgcaaaaaagagactcaattttgggcagactggcgactcagagtcagtccccgaccctcaaccaatcggagaaccaccagcaggcccctctggtctgggatctggtacaatggctgcaggcggtggcgctccaatggcagacaataacgaaggcgccgacggagtgggtagttcctcaggaaattggcattgcgattccacatggctgggcgacagagtcatcaccaccagcacccgcacctgggccctgcccacctacaacaaccacctctacaagcaaatctccaacgggacctcgggaggaagcaccaacgacaacacctacttcggctacagcaccccctgggggtattttgacttcaacagattccactgccacttttcaccacgtgactggcagcgactcatcaacaacaactggggattccggcccaagaggctcaacttcaagctcttcaacatccaagtcaaggaggtcacgcagaatgaaggcaccaagaccatcgccaataaccttaccagcacgattcaggtctttacggactcggaataccagctcccgtacgtgctcggctcggcgcaccagggctgcctgcctccgttcccggcggacgtcttcatgattcctcagtacgggtacctgactctgaacaatggcagtcaggctgtgggccggtcgtccttctactgcctggagtactttccttctcaaatgctgagaacgggcaacaactttgaattcagctacaacttcgaggacgtgcccttccacagcagctacgcgcacagccagagcctggaccggctgatgaaccctctcatcgaccagtacttgtactacctgtcccggactcaaagcacgggcggtactgcaggaactcagcagttgctattttctcaggccgggcctaacaacatgtcggctcaggccaagaactggctacccggtccctgctaccggcagcaacgtgtctccacgacactgtcgcagaacaacaacagcaactttgcctggacgggtgccaccaagtatcatctgaatggcagagactctctggtgaatcctggcgttgccatggctacccacaaggacgacgaagagcgattttttccatccagcggagtcttaatgtttgggaaacagggagctggaaaagacaacgtggactatagcagcgtgatgctaaccagcgaggaagaaataaagaccaccaacccagtggccacagaacagtacggcgtggtggccgataacctgcaacagcaaaacgccgctcctattgtaggggccgtcaatagtcaaggagccttacctggcatggtgtggcagaaccgggacgtgtacctgcagggtcccatctgggccaagattcctcatacggacggcaactttcatccctcgccgctgatgggaggctttggactgaagcatccgcctcctcagatcctgattaaaaacacacctgttcccgcggatcctccgaccaccttcaatcaggccaagctggcttctttcatcacgcagtacagtaccggccaggtcagcgtggagatcgagtgggagctgcagaaggagaacagcaaacgctggaacccagagattcagtacacttccaactactacaaatctacaaatgtggactttgctgtcaatactgagggtacttattccgagcctcgccccattggcacccgttacctcacccgtaatctgtaa

SEQ ID NO: 11 - protein, 738 aa, Adeno-associated virus
MAADGYLPDWLEDNLSEGIREWWDLKPGAPKPKANQQKQDNGRGLVLPGYKYLGPFNGLDKGEPVNAADAAALEHDKAYDQQLQAGDNPYLRYNHADAEFQERLQEDTSFGGNLGRAVFQAKKRVLEPLGLVESPVKTAPGKKRPVEPSPQRSPDSSTGIGKKGQQPAKKRLNFGQTGDSESVPDPQPIGEPPAGPSGLGSGTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLNFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFEFSYNFEDVPFHSSYAHSQSLDRLMNPLIDQYLYYLSRTQSTGGTAGTQQLLFSQAGPNNMSAQAKNWLPGPCYRQQRVSTTLSQNNNSNFAWTGATKYHLNGRDSLVNPGVAMATHKDDEERFFPSSGVLMFGKQGAGKDNVDYSSVMLTSEEEIKTTNPVATEQYGVVADNLQQQNAAPIVGAVNSQGALPGMVWQNRDVYLQGPIWAKIPHTDGNFHPSPLMGGFGLKHPPPQILIKNTPVPADPPTTFTKAKLASFITQYSTGQVSVEIEWELQKENSKRWNPEIQYTSNYYKSTNVDFAVNTEGTYSEPRPIGTRYLTRNL

SEQ ID NO: 12 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGHDNVDADKVMITNEEEIKTTNPVATEAYGTVATNKQSAYAQAQTGALQSQGVLPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 13 - DNA, 2211 bp, synthetic construct
  source 1..2211: mol_type = other DNA
atggctgccgatggttatcttccagattggctcgaggacaaccttagtgaaggtattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggacacgacaacgtggatgcggacaaagtcatgataaccaacgaagaagaaattaaaactactaacccggtagcaacggaggcatatggaaccgtggccacaaacaaacagagtgcctatgcacaggcgcagaccggcgcattgcaaagccaaggagttcttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaa

SEQ ID NO: 14 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGHNNVDADKVMITNEEEIKTTNPVATEAYGTVATNKQSAYAQAQTGALQSQGVLPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 15 - DNA, 2211 bp, synthetic construct
  source 1..2211: mol_type = other DNA
atggctgccgatggttatcttccagattggctcgaggacaaccttagtgaaggtattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggacataataacgtggatgcggacaaagtcatgataaccaacgaagaagaaattaaaactactaacccggtagcaacggaggcctatggaactgtggccacaaacaagcagagtgcctacgcacaggcgcagaccggcgctttacaaagccaaggagtccttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaa

SEQ ID NO: 16 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGHDNVDADKVMITNEEEIKTTNPVATESYGTVATNKQNHTQQAQTGALQSQGVLPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 17 - DNA, 2211 bp, synthetic construct
  source 1..2211: mol_type = other DNA
atggctgccgatggttatcttccagattggctcgaggacaaccttagtgaaggtattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggacatgacaacgtggatgcggacaaagtcatgataaccaacgaagaagaaattaaaactactaacccggtagcaacggagtcctatggaaccgtggccacaaacaagcagaatcatacccagcaggcgcagaccggcgccctacaatcacaaggagtccttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaa

SEQ ID NO: 18 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGIVATNHQSSNTQANVGALQSQGVLPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 19 - DNA, 2211 bp, synthetic construct
  source 1..2211: mol_type = other DNA
atggctgccgatggttatcttccagattggctcgaggacaaccttagtgaaggtattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggaatagtggccacaaaccaccagagtagcaatacgcaggcgaatgtgggcgcgttgcaatcgcaaggagtgcttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaa

SEQ ID NO: 20-99 - skipped (80 entries)

SEQ ID NO: 100 - DNA, 798 bp, unidentified
  source 1..798: mol_type = unassigned DNA
ccaacctgaaaaaaagtgatttcaggcaggtgctccaggtaattaaacattaataccccaccaaccaaccatcccttaaacccttacctcttgctcagctaattacagcccggaggagaagggccgtcccgcccgctcacctgtgggagtaacgcggtcagtcagagccggggcgggcggcgcgaggcggcggcggagcggggcacggggcgaaggcagcgcgcagcgactcccgcccgccgcgcgcttcgctttttatagggccgccgccgccgccgcctcgccataaaaggaaactttcggagcgcgccgctctgattggctgccgccgcacctctccgcctcgccccgccccgcccctcgccccgccccgccccgcctggcgcgcgccccccccccccccccgcccccatcgctgcacaaaataattaaaaaataaataaatacaaaattgggggtggggaggggggggagatggggagagtgaagcagaacgtggggctcacctcgaccatggtaatagcgatgactaatacgtagatgtactgccaagtaggaaagtcccataaggtcatgtactgggcacaatgccaggcgggccatttaccgtcattgacgtcaatagggggcgtacttggcatatgatacacttgatgtactgccaagtgggcagtttaccgtaaatactccacccattgacgtcaatggaaagtccctattggcgttactattgacgtcaatgggcgggggtcgttgggcggtcagccaggcgggccatttaccgtaagttatgtaacg

SEQ ID NO: 101 - DNA, 1181 bp, Homo sapiens
  source 1..1181: mol_type = genomic DNA
ttggctccggtgcccgtcagtgggcagagcgcacatcgcccacagtccccgagaagttgtggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtga

SEQ ID NO: 102 - DNA, 448 bp, Homo sapiens
  source 1..448: mol_type = genomic DNA
agtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcag

SEQ ID NO: 103 - protein, 9 aa, unidentified
PLNGAVHLY

SEQ ID NO: 104 - protein, 7 aa, unidentified
IVMNSLK

SEQ ID NO: 105 - protein, 7 aa, unidentified
RDSPKGW

SEQ ID NO: 106 - protein, 7 aa, unidentified
YSTDVRM

SEQ ID NO: 107 - protein, 7 aa, unidentified
RESPRGL

SEQ ID NO: 108 - protein, 7 aa, unidentified
GNNTRSV

SEQ ID NO: 109 - protein, 7 aa, unidentified
GNNTRDT

SEQ ID NO: 110 - protein, 7 aa, unidentified
TNSTRPV

SEQ ID NO: 111 - protein, 35 aa, unidentified
SLEEEWAQVECEVYGRGCPSGSLDESFYDWFERQL

SEQ ID NO: 112 - protein, 96 aa, unidentified
TPFYPVGYTVKQPGTCGDGVLQPGEECDDGNPDVSDGCIDCHRAYCGDGYRHQGVEDCDGSDFGYLTCETYLPGSYGDLRCTQYCSIDSTPCRYFT

SEQ ID NO: 113 - protein, 7 aa, unidentified
RGDLGLS

SEQ ID NO: 114 - protein, 7 aa, unidentified
RGDLSTP

SEQ ID NO: 115 - protein, 10 aa, unidentified
SNSRGDYNSL

SEQ ID NO: 116 - protein, 10 aa, unidentified
ENRRGDFNNT

SEQ ID NO: 117 - protein, 8 aa, unidentified
SRGDYNSL

SEQ ID NO: 118 - protein, 7 aa, unidentified
RGDYNSL

SEQ ID NO: 119 - protein, 6 aa, unidentified
RGDLST

SEQ ID NO: 120 - protein, 7 aa, unidentified
RGDYVGL

SEQ ID NO: 121 - protein, 7 aa, unidentified
RGDAVGV

SEQ ID NO: 122-199 - skipped (78 entries)

SEQ ID NO: 200 - protein, 13 aa, synthetic construct
AYGTVATNKQSAY

SEQ ID NO: 201 - protein, 52 aa, synthetic construct
HDNVDADKVMITNEEEIKTTNPVATEAYGTVATNKQSAYAQAQTGALQSQGV

SEQ ID NO: 202 - protein, 52 aa, synthetic construct
HNNVDADKVMITNEEEIKTTNPVATEAYGTVATNKQSAYAQAQTGALQSQGV

SEQ ID NO: 203 - protein, 10 aa, synthetic construct
TVATNKQNHT

SEQ ID NO: 204 - protein, 52 aa, synthetic construct
HDNVDADKVMITNEEEIKTTNPVATESYGTVATNKQNHTQQAQTGALQSQGV

SEQ ID NO: 205 - protein, 10 aa, synthetic construct
IVATNHQSSN

SEQ ID NO: 206 - protein, 23 aa, synthetic construct
IVATNHQSSNTQANVGALQSQGV

SEQ ID NO: 207 - protein, 4 aa, synthetic construct
NHTQ

SEQ ID NO: 208 - protein, 7 aa, unidentified
ASSLNIA

SEQ ID NO: 209 - protein, 4 aa, synthetic construct
RGDY

SEQ ID NO: 210 - protein, 4 aa, synthetic construct
RGDF

SEQ ID NO: 211 - protein, 4 aa, synthetic construct
GGGS

SEQ ID NO: 212 - protein, 289 aa, synthetic construct
MRHIICHGGVITEEMAASLLDQLIEEVLADNLPPPSHFEPPTLHELYDLDVTAPEDPNEEAVSQIFPDSVMLAVQEGIDLLTFPPAPGSPEPPHLSRQPEQPEQRALGPVSMPNLVPEVIDLTCHEAGFPPSDDEDEEGEEFVLDYVEHPGHGCRSCHYHRRNTGDPDIMCSLCYMRTCGMFVYSPVSEPEPEPEPEPEPARPTRRPKMAPAILRRPTSPVSRECNSSTDSCDSGPSNTPPEIHPVVPLCPIKPVAVRVGGRRQAVECIEDLLNEPGQPLDLSCKRPRP

SEQ ID NO: 213 - protein, 176 aa, synthetic construct
MEAWECLEDFSAVRNLLEQSSNSTSWFWRFLWGSSQAKLVCRIKEDYKWEFEELLKSCGELFDSLNLGHQALFQEKVIKTLDFSTPGRAAAAVAFLSFIKDKWSEETHLSGGYLLDFLAMHLWRAVVRHKNRLLLLSSVRPAIIPTEEQQQQQEEARRRRQEQSPWNPRAGLDPRE

SEQ ID NO: 214 - protein, 496 aa, synthetic construct
MERRNPSERGVPAGFSGHASVESGCETQESPATVVFRPPGDNTDGGAAAAAGGSQAAAAGAEPMEPESRPGPSGMNVVQVAELYPELRRILTITEDGQGLKGVKRERGACEATEEARNLAFSLMTRHRPECITFQQIKDNCANELDLLAQKYSIEQLTTYWLQPGDDFEEAIRVYAKVALRPDCKYKISKLVNIRNCCYISGNGAEVEIDTEDRVAFRCSMINMWPGVLGMDGVVIMNVRFTGPNFSGTVFLANTNLILHGVSFYGFNNTCVEAWTDVRVRGCAFYCCWKGVVCRPKSRASIKKCLFERCTLGILSEGNSRVRHNVASDCGCFMLVKSVAVIKHNMVCGNCEDRASQMLTCSDGNCHLLKTIHVASHSRKAWPVFEHNILTRCSLHLGNRRGVFLPYQCNLSHTKILLEPESMSKVNLNGVFDMTMKIWKVLRYDETRTRCRPCECGGKHIRNQPVMLDVTEELRPDHLVLACTRAEFGSSDEDTD

SEQ ID NO: 215 - protein, 107 aa, synthetic construct
MLSGEAEQLRLKHLVHCRRHKCFARDSGEFCYFELPEDHIEGPAHGVRLTAQGELARSLIREFTQRPLLVERDRGPCVLTVICNCPNLGLHQDLCCHLCAEYNKYRN

SEQ ID NO: 216 - protein, 63 aa, synthetic construct
MNNSSNSTGYSNSGFSRIGVGVILCLVILFILILTLLCLRLAACCVHICIYCQLFKRWGRHPR

SEQ ID NO: 217 - protein, 160 aa, synthetic construct
MIRYIILGLLTLASAHGTTQKVDFKEPACNVTFAAEANECTTLIKCTTEHEKLLIRHKNKIGKYAVYAIWQPGDTTEYNVTVFQGKSHKTFMYTFPFYEMCDITMYMSKQYKLWPPQNCVENTGTFCCTAMLITVLALVCTLLYIKYKSRRSFIEEKKMP

SEQ ID NO: 218 - protein, 93 aa, synthetic construct
MTNTTNAAAATGLTSTTNTPQVSAFVNNWDNLGMWWFSIALMFVCLIIMWLICCLKRKRARPPIYSPIIVLHPNNDGIHRLDGLKHMFFSLTV

SEQ ID NO: 219 - protein, 91 aa, synthetic construct
MIPRVFILLTLVALFCACSTLAAVSHIEVDCIPAFTVYLLYGFVTLTLICSLITVVIAFIQCIDWVCVRFAYLRHHPQYRDRTIAELLRIL

SEQ ID NO: 220 - protein, 132 aa, synthetic construct
MKFTVTFLLIICTLSAFCSPTSKPQRHISCRFTRIWNIPSCYNEKSDLSEAWLYAIISVMVFCSTILALAIYPYLDIGWKRIDAMNHPTFPAPAMLPLQQVVAGGFVPANQPRPTSPTPTEISYFNLTGGDD

SEQ ID NO: 221 - protein, 128 aa, synthetic construct
MTDTLDLEMDGIITEQRLLERRRAAAEQQRMNQELQDMVNLHQCKRGIFCLVKQAKVTYDSNTTGHRLSYKLPTKRQKLVVMVGEKPITITQHSVETEGCIHSPCQGPEDLCTLIKTLCGLKDLIPFN

SEQ ID NO: 222 - protein, 150 aa, synthetic construct
MTTSGVPFGMTLRPTRSRLSRRTPYSRDRLPPFETETRATILEDHPLLPECNTLTMHNAWTSPSPPVKQPQVGQQPVAQQLDSDMNLSELPGEFINITDERLARQETVWNITPKNMSVTHDMMLFKASRGERTVYSVCWEGGGRLNTRVL

SEQ ID NO: 223 - protein, 294 aa, synthetic construct
MTTSGVPFGMTLRPTRSRLSRRTPYSRDRLPPFETETRATILEDHPLLPECNTLTMHNVSYVRGLPCSVGFTLIQEWVVPWDMVLTREELVILRKCMHVCLCCANIDIMTSMMIHGYESWALHCHCSSPGSLQCIAGGQVLASWFRMVVDGAMFNQRFIWYREVVNYNMPKEVMFMSSVFMRGRHLIYLRLWYDGHVGSVVPAMSFGYSALHCGILNNIVVLCCSYCADLSEIRVRCCARRTRRLMLRAVRIIAEETTAMLYSCRTERRRQQFIRALLQHHRPILMHDYDSTPM

SEQ ID NO: 224 - protein, 114 aa, synthetic construct
MVLPALPAPPVCDSQNECVGWLGVAYSAVVDVIRAAAHEGVYIEPEARGRLDALREWIYYNYYTERSKRRDRRRRSVCHARTWFCFRKYDYVRRSIWHDTTTNTISVVSAHSVQ

SEQ ID NO: 225 - protein, 116 aa, synthetic construct
MIRCLRLKVEGALEQIFTMAGLNIRDLLRDILRRWRDENYLGMVEGAGMFIEEIHPEGFSLYVHLDVRAVCLLEAIVQHLTNAIICSLAVEFDHATGGERVHLIDLHFEVLDNLLE

SEQ ID NO: 226 - protein, 130 aa, synthetic construct
MFERKMVSFSVVVPELTCLYLHEHDYDVLSFLREALPDFLSSTLHFISPPMQQAYIGATLVSIAPSMRVIISVGSFVMVPGGEVAALVRADLHDYVQLALRRDLRDRGIFVNVPLLNLIQVCEEPEFLQS

SEQ ID NO: 227 - protein, 128 aa, synthetic construct
MAAAVEALYVVLEREGAILPRQEGFSGVYVFFSPINFVIPPMGAVMLSLRLRVCIPPGYFGRFLALTDVNQPDVFTESYIMTPDMTEELSVVLFNHGDQFFYGHAGMAVVRLMLIRVVFPVVRQASNV

SEQ ID NO: 228 - protein, 289 aa, synthetic construct
MRHIICHGGVITEEMAASLLDQLIEEVLADNLPPPSHFEPPTLHELYDLDVTAPEDPNEEAVSQIFPESVMLAVQEGIDLFTFPPAPGSPEPPHLSRQPEQPEQRALGPVSMPNLVPEVIDLTCHEAGFPPSDDEDEEGEEFVLDYVEHPGHGCRSCHYHRRNTGDPDIMCSLCYMRTCGMFVYSPVSEPEPEPEPEPEPARPTRRPKLVPAILRRPTSPVSRECNSSTDSCDSGPSNTPPEIHPVVPLCPIKPVAVRVGGRRQAVECIEDLLNESGQPLDLSCKRPRP

SEQ ID NO: 229 - protein, 175 aa, synthetic construct
MEAWECLEDFSAVRNLLEQSSNSTSWFWRFLWGSSQAKLVCRIKEDYKWEFEELLKSCGELFDSLNLGHQALFQEKVIKTLDFSTPGRAAAAVAFLSFIKDKWSEETHLSGGYLLDFLAMHLWRAVVRHKNRLLLLSSVRPAIIPTEEQQQEEARRRRRQEQSPWNPRAGLDPRE

SEQ ID NO: 230 - protein, 495 aa, synthetic construct
MERRNPSERGVPAGFSGHASVESGGETQESPATVVFRPPGNNTDGGATAGGSQAAAAAGAEPMEPESRPGPSGMNVVQVAELFPELRRILTINEDGQGLKGVKRERGASEATEEARNLTFSLMTRHRPECVTFQQIKDNCANELDLLAQKYSIEQLTTYWLQPGDDFEEAIRVYAKVALRPDCKYKISKLVNIRNCCYISGNGAEVEIDTEDRVAFRCSMINMWPGVLGMDGVVIMNVRFTGPNFSGTVFLANTNLILHGVSFYGFNNTCVEAWTDVRVRGCAFYCCWKGVVCRPKSRASIKKCLFERCTLGILSEGNSRVRHNVASDCGCFMLVKSVAVIKHNMVCGNCEDRASQMLTCSDGNCHLLKTIHVASHSRKAWPVFEHNILTRCSLHLGNRRGVFLPYQCNLSHTKILLEPESMSKVNLNGVFDMTMKIWKVLRYDETRTRCRPCECGGKHIRNQPVMLDVTEELRPDHLVLACTRAEFGSSDEDTD

SEQ ID NO: 231 - protein, 107 aa, synthetic construct
MTSGEAERLRLTHLDHCRRHKCFARGSGEFCYFELPEEHIEGPAHGVRLTTQVELTRSLIREFTKRPLLVERERGPCVLTVVCNCPNPGLHQDLCCHLCAEYNKYRN

SEQ ID NO: 232 - protein, 61 aa, synthetic construct
MSNSSNSTSLSNFSGIGVGVILTLVILFILILALLCLRVAACCTHVCTYCQLFKRWGQHPR

SEQ ID NO: 233 - protein, 159 aa, synthetic construct
MRYMILGLLALAAVCSAAKKVEFKEPACNVTFKSEANECTTLIKCTTEHEKLIIRHKDKIGKYAVYAIWQPGDTNDYNVTVFQGENRKTFMYKFPFYEMCDITMYMSKQYKLWPPQKCLENTGTFCSTALLITALALVCTLLYLKYKSRRSFIDEKKMP

SEQ ID NO: 234 - protein, 101 aa, synthetic construct
MTGSTIAPTTDYRNTTATGLTSALNLPQVHAFVNDWASLDMWWFSIALMFVCLIIMWLICCLKRRRARPPIYRPIIVLNPHNEKIHRLDGLKPCSLLLQYD

SEQ ID NO: 235 - protein, 91 aa, synthetic construct
MIPRVLILLTLVALFCACSTLAAVAHIEVDCIPPFTVYLLYGFVTLILICSLVTVVIAFIQFIDWVCVRIAYLRHHPQYRDRTIADLLRIL

SEQ ID NO: 236 - protein, 130 aa, synthetic construct
MKRSVIFVLLIFCALPVLCSQTSAPPKRHISCRFTQIWNIPSCYNKQSDLSEAWLYAIISVMVFCSTIFALAIYPYLDIGWNAIDAMNHPTFPVPAVIPLQQVIAPINQPRPPSPTPTEISYFNLTGGDD

SEQ ID NO: 237 - protein, 128 aa, synthetic construct
MTESLDLELDGINTEQRLLERRKAASERERLKQEVEDMVNLHQCKRGIFCVVKQAKLTYEKTTTGNRLSYKLPTQRQKLVLMVGEKPITVTQHSAETEGCLHFPYQGPEDLCTLIKTMCGIRDLIPFN

SEQ ID NO: 238 - protein, 150 aa, synthetic construct
MTTSGVPFGMTLRPTRSRLSRRTPYSRDRLPPFETETRATILEDHPLLPECNTLTMHNAWTSPSPPVEQPQVGQQPVAQQLDSDMNLSELPGEFINITDERLARQETVWNITPKNMSVTHDMMLFKASRGERTVYSVCWEGGGRLNTRVL

SEQ ID NO: 239 - protein, 114 aa, synthetic construct
MVLPALPAPPVCDSQNECVGWLGVAYSAVVDVIRAAAHEGVYIEPEARGRLDALREWIYYNYYTERAKRRDRRRRSVCHARTWFCFRKYDYVRRSIWHDTTTNTISVVSAHSVQ

SEQ ID NO: 240 - protein, 116 aa, synthetic construct
MIRCLRLKVEGALEQIFTMAGLNIRDLLRDILIRWRDENYLGMVEGAGMFIEEIHPEGFSLYVHLDVRAVCLLEAIVQHLTNAIICSLAVEFDHATGGERVHLIDLHFEVLDNLLE

SEQ ID NO: 241 - protein, 130 aa, synthetic construct
MFERKMVSFSVVVPELTCLYLHEHDYDVLAFLREALPDFLSSTLHFISPPMQQAYIGATLVSIAPSMRVIISVGSFVMVPGGEVAALVRADLHDYVQLALRRDLRDRGIFVNVPLLNLIQVCEEPEFLQS

SEQ ID NO: 242 - protein, 621 aa, synthetic construct
MPGFYEIVIKVPSDLDGHLPGISDSFVNWVAEKEWELPPDSDMDLNLIEQAPLTVAEKLQRDFLTEWRRVSKAPEALFFVQFEKGESYFHMHVLVETTGVKSMVLGRFLSQIREKLIQRIYRGIEPTLPNWFAVTKTRNGAGGGNKVVDECYIPNYLLPKTQPELQWAWTNMEQYLSACLNLTERKRLVAQHLTHVSQTQEQNKENQNPNSDAPVIRSKTSARYMELVGWLVDKGITSEKQWIQEDQASYISFNAASNSRSQIKAALDNAGKIMSLTKTAPDYLVGQQPVEDISSNRIYKILELNGYDPQYAASVFLGWATKKFGKRNTIWLFGPATTGKTNIAEAIAHTVPFYGCVNWTNENFPFNDCVDKMVIWWEEGKMTAKVVESAKAILGGSKVRVDQKCKSSAQIDPTPVIVTSNTNMCAVIDGNSTTFEHQQPLQDRMFKFELTRRLDHDFGKVTKQEVKDFFRWAKDHVVEVEHEFYVKKGGAKKRPAPSDADISEPKRVRESVAQPSTSDAEASINYADRYQNKCSRHVGMNLMLFPCRQCERMNQNSNICFTHGQKDCLECFPVSESQPVSVVKKAYQKLCYIHHIMGKVPDACTACDLVNVDLDDCIFEQ

SEQ ID NO: 243 - protein, 536 aa, synthetic construct
MPGFYEIVIKVPSDLDGHLPGISDSFVNWVAEKEWELPPDSDMDLNLIEQAPLTVAEKLQRDFLTEWRRVSKAPEALFFVQFEKGESYFHMHVLVETTGVKSMVLGRFLSQIREKLIQRIYRGIEPTLPNWFAVTKTRNGAGGGNKVVDECYIPNYLLPKTQPELQWAWTNMEQYLSACLNLTERKRLVAQHLTHVSQTQEQNKENQNPNSDAPVIRSKTSARYMELVGWLVDKGITSEKQWIQEDQASYISFNAASNSRSQIKAALDNAGKIMSLTKTAPDYLVGQQPVEDISSNRIYKILELNGYDPQYAASVFLGWATKKFGKRNTIWLFGPATTGKTNIAEAIAHTVPFYGCVNWTNENFPFNDCVDKMVIWWEEGKMTAKVVESAKAILGGSKVRVDQKCKSSAQIDPTPVIVTSNTNMCAVIDGNSTTFEHQQPLQDRMFKFELTRRLDHDFGKVTKQEVKDFFRWAKDHVVEVEHEFYVKKGGAKKRPAPSDADISEPKRVRESVAQPSTSDAEASINYADRLARGHSL

SEQ ID NO: 244 - protein, 397 aa, synthetic construct
MELVGWLVDKGITSEKQWIQEDQASYISFNAASNSRSQIKAALDNAGKIMSLTKTAPDYLVGQQPVEDISSNRIYKILELNGYDPQYAASVFLGWATKKFGKRNTIWLFGPATTGKTNIAEAIAHTVPFYGCVNWTNENFPFNDCVDKMVIWWEEGKMTAKVVESAKAILGGSKVRVDQKCKSSAQIDPTPVIVTSNTNMCAVIDGNSTTFEHQQPLQDRMFKFELTRRLDHDFGKVTKQEVKDFFRWAKDHVVEVEHEFYVKKGGAKKRPAPSDADISEPKRVRESVAQPSTSDAEASINYADRYQNKCSRHVGMNLMLFPCRQCERMNQNSNICFTHGQKDCLECFPVSESQPVSVVKKAYQKLCYIHHIMGKVPDACTACDLVNVDLDDCIFEQ

SEQ ID NO: 245 - protein, 312 aa, synthetic construct
MELVGWLVDKGITSEKQWIQEDQASYISFNAASNSRSQIKAALDNAGKIMSLTKTAPDYLVGQQPVEDISSNRIYKILELNGYDPQYAASVFLGWATKKFGKRNTIWLFGPATTGKTNIAEAIAHTVPFYGCVNWTNENFPFNDCVDKMVIWWEEGKMTAKVVESAKAILGGSKVRVDQKCKSSAQIDPTPVIVTSNTNMCAVIDGNSTTFEHQQPLQDRMFKFELTRRLDHDFGKVTKQEVKDFFRWAKDHVVEVEHEFYVKKGGAKKRPAPSDADISEPKRVRESVAQPSTSDAEASINYADRLARGHSL

SEQ ID NO: 246-299 - skipped (54 entries)

SEQ ID NO: 300 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGHDNVDADKVMITNEEEIKTTNPVATEAYGTVATNKQSAYAQAQTGALQSQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 301 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGHDNVDADKVMITNEEEIKTTNPVATEAYGQVATNKQSAYAQAQTGALQSQGVLPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 302 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGHDNVDADKVMITNEEEIKTTNPVATEAYGTVATNHQSAYAQAQTGALQSQGVLPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 303 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATEAYGTVATNKQSAYAQAQTGALQSQGVLPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 304 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGHDNVDADKVMITNEEEIKTTNPVATESYGTVATNKQSAYAQAQTGALQSQGVLPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 305 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGHDNVDADKVMITNEEEIKTTNPVATEAYGTVATNKQSAYAQAQTGWLQSQGVLPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 306 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGHDNVDADKVMITNEEEIKTTNPVATEAYGTVATNKQSAYAQAQTGALQNQGVLPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 307 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGHDNVDADKVMITNEEEIKTTNPVATEAYGTVATNKQSAYAQAQTGAVQSQGVLPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 308 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGHDNVDADKVMITNEEEIKTTNPVATEAYGTVATNKQSAQAQAQTGALQSQGVLPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 309 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGHDNVDADKVMITNEEEIKTTNPVATESYGQVATNKQSAYAQAQTGALQSQGVLPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 310 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGHDNVDADKVMITNEEEIKTTNPVATEAYGQVATNKQSAYAQAQTGALQNQGVLPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 311 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGHDNVDADKVMITNEEEIKTTNPVATEAYGTVATNHQSAYAQAQTGAVQSQGVLPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 312 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGHDNVDADKVMITNEEEIKTTNPVATESYGTVATNHQSAYAQAQTGALQSQGVLPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 313 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGHDNVDADKVMITNEEEIKTTNPVATEAYGTVATNKQSAQAQAQTGALQSQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 314 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATEAYGTVATNKQSAYAQAQTGALQSQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 315 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATEAYGQVATNKQSAYAQAQTGALQSQGVLPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 316 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGTVATNKQSAYAQAQTGALQSQGVLPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 317 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATEAYGTVATNKQSAYAQAQTGAVQSQGVLPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 318 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGHDNVDADKVMITNEEEIKTTNPVATEAYGQVATNHQSAYAQAQTGALQSQGVLPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 319 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGHDNVDADKVMITNEEEIKTTNPVATEAYGTVATNKQSAYAQAQTGAVQNQGVLPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 320 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGHDNVDADKVMITNEEEIKTTNPVATEAYGTVATNHQSAYAQAQTGALQSQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 321 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGHDNVDADKVMITNEEEIKTTNPVATESYGTVATNKQSAYAQAQTGALQSQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 322 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGHDNVDADKVMITNEEEIKTTNPVATEAYGTVATNKQSAYAQAQTGALQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 323 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGHDNVDADKVMITNEEEIKTTNPVATEAYGQVATNKQSAYAQAQTGALQSQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 324 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGHDNVDADKVMITNEEEIKTTNPVATESYGTVATNKQSAYAQAQTGALQNQGVLPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 325 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATEAYGTVATNKQSAQAQAQTGALQSQGVLPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 326 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGHDNVDADKVMITNEEEIKTTNPVATEAYGTVATNHQSAYAQAQTGALQNQGVLPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 327 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGHDNVDADKVMITNEEEIKTTNPVATEAYGQVATNKQSAYAQAQTGAVQSQGVLPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 328 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATEAYGTVATNKQSAYAQAQTGALQNQGVLPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 329 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGHDNVDADKVMITNEEEIKTTNPVATEAYGTVATNKQSAQAQAQTGWLQSQGVLPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 330 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATEAYGTVATNHQSAYAQAQTGALQSQGVLPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 331 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGHDNVDADKVMITNEEEIKTTNPVATEAYGTVATNHQSAQAQAQTGALQSQGVLPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 332 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGHDNVDADKVMITNEEEIKTTNPVATESYGTVATNKQSAQAQAQTGALQSQGVLPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 333 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGTVATNHQSAYAQAQTGALQSQGVLPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 334 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNKQSAYAQAQTGALQSQGVLPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 335 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGHDNVDADKVMITNEEEIKTTNPVATEAYGTVATNHQSAYAQAQTGAVQSQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 336 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGHDNVDADKVMITNEEEIKTTNPVATEAYGQVATNKQSAYAQAQTGAVQNQGVLPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 337 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGHDNVDADKVMITNEEEIKTTNPVATESYGQVATNKQSAYAQAQTGALQNQGVLPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 338 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATEAYGTVATNKQSAYAQAQTGAVQSQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 339 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGHDNVDADKVMITNEEEIKTTNPVATESYGQVATNKQSAYAQAQTGALQSQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 340 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATEAYGTVATNKQSAYAQAQTGAVQNQGVLPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 341 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGHDNVDADKVMITNEEEIKTTNPVATEAYGTVATNKQSAQAQAQTGWVQSQGVLPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 342 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATEAYGQVATNHQSAYAQAQTGALQSQGVLPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 343 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGHDNVDADKVMITNEEEIKTTNPVATEAYGTVATNKQSAYAQAQTGAVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 344 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATEAYGQVATNKQSAYAQAQTGALQSQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 345 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATEAYGTVATNKQSAYAQAQTGWVQSQGVLPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 346 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATEAYGTVATNHQSAYAQAQTGALQSQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 347 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATEAYGQVATNKQSAYAQAQTGALQNQGVLPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 348 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGHDNVDADKVMITNEEEIKTTNPVATESYGTVATNKQSAYAQAQTGAVQNQGVLPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 349 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGHDNVDADKVMITNEEEIKTTNPVATEAYGQVATNKQSAYAQAQTGAVQSQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 350 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGHDNVDADKVMITNEEEIKTTNPVATEAYGTVATNHQSAYAQAQTGAVQNQGVLPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 351 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATEAYGTVATNKQSAYAQAQTGALQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 352 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGHDNVDADKVMITNEEEIKTTNPVATEAYGTVATNHQSAYAQAQTGALQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 353 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGHDNVDADKVMITNEEEIKTTNPVATESYGQVATNKQSAYAQAQTGAVQSQGVLPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 354 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGHDNVDADKVMITNEEEIKTTNPVATESYGTVATNHQSAYAQAQTGALQNQGVLPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 355 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGHDNVDADKVMITNEEEIKTTNPVATESYGTVATNHQSAYAQAQTGALQSQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 356 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGHDNVDADKVMITNEEEIKTTNPVATEAYGQVATNHQSAYAQAQTGALQSQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 357 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATEAYGTVATNHQSAYAQAQTGALQNQGVLPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 358 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGTVATNKQSAQAQAQTGALQSQGVLPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 359 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGHDNVDADKVMITNEEEIKTTNPVATESYGTVATNKQSAYAQAQTGAVQSQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 360 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATEAYGTVATNHQSAQAQAQTGALQSQGVLPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 361 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATEAYGTVATNHQSAYAQAQTGAVQSQGVLPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 362 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGTVATNKQSAYAQAQTGALQSQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 363 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGHDNVDADKVMITNEEEIKTTNPVATESYGQVATNHQSAYAQAQTGALQSQGVLPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 364 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGHDNVDADKVMITNEEEIKTTNPVATEAYGQVATNHQSAYAQAQTGALQNQGVLPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 365 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGHDNVDADKVMITNEEEIKTTNPVATESYGTVATNKQSAYAQAQTGALQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 366 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGHDNVDADKVMITNEEEIKTTNPVATESYGTVATNKQSAQAQAQTGWLQSQGVLPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 367 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGHDNVDADKVMITNEEEIKTTNPVATESYGTVATNHQSAYAQAQTGAVQSQGVLPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 368 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGHDNVDADKVMITNEEEIKTTNPVATEAYGQVATNKQSAYAQAQTGALQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 369 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATEAYGQVATNKQSAYAQAQTGAVQSQGVLPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 370 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGHDNVDADKVMITNEEEIKTTNPVATESYGTVATNHQSAQAQAQTGALQSQGVLPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 371 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGHDNVDADKVMITNEEEIKTTNPVATESYGTVATNKQSAQAQAQTGALQSQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 372 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGHDNVDADKVMITNEEEIKTTNPVATESYGTVATNKQSAYAQAQTGAVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 373 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATEAYGQVATNKQSAYAQAQTGALQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 374 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNKQSAYAQAQTGALQNQGVLPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 375 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGTVATNKQSAYAQAQTGAVQNQGVLPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 376 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATEAYGTVATNKQSAYAQAQTGAVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 377 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGTVATNHQSAYAQAQTGALQSQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 378 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGHDNVDADKVMITNEEEIKTTNPVATESYGQVATNHQSAYAQAQTGALQSQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 379 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGTVATNKQSAQAQAQTGWLQSQGVLPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 380 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGHDNVDADKVMITNEEEIKTTNPVATEAYGTVATNHQSAYAQAQTGAVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 381 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGHDNVDADKVMITNEEEIKTTNPVATESYGTVATNKQSAQAQAQTGWVQSQGVLPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 382 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGHDNVDADKVMITNEEEIKTTNPVATESYGQVATNHQSAYAQAQTGAVQSQGVLPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 383 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGTVATNKQSAYAQAQTGALQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 384 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGHDNVDADKVMITNEEEIKTTNPVATEAYGQVATNHQSAYAQAQTGALQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 385 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGHDNVDADKVMITNEEEIKTTNPVATEAYGQVATNKQSAYAQAQTGAVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 386 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATEAYGTVATNHQSAYAQAQTGAVQNQGVLPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 387 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGTVATNHQSAQAQAQTGALQSQGVLPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 388 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGHDNVDADKVMITNEEEIKTTNPVATESYGQVATNKQSAYAQAQTGAVQNQGVLPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 389 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGHDNVDADKVMITNEEEIKTTNPVATESYGQVATNHQSAYAQAQTGALQNQGVLPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 390 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATEAYGQVATNHQSAYAQAQTGALQNQGVLPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 391 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGHDNVDADKVMITNEEEIKTTNPVATEAYGQVATNHQSAYAQAQTGAVQSQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 392 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGTVATNHQSAYAQAQTGAVQSQGVLPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 393 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGHDNVDADKVMITNEEEIKTTNPVATEAYGQVATNHQSAYAQAQTGAVQNQGVLPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 394 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATEAYGQVATNHQSAYAQAQTGALQSQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 395 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGHDNVDADKVMITNEEEIKTTNPVATESYGQVATNKQSAYAQAQTGALQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 396 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGTVATNHQSAYAQAQTGALQNQGVLPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 397 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATEAYGQVATNHQSAYAQAQTGAVQSQGVLPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 398 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGHDNVDADKVMITNEEEIKTTNPVATESYGTVATNHQSAYAQAQTGALQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 399 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATEAYGTVATNHQSAYAQAQTGAVQSQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 400 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGHDNVDADKVMITNEEEIKTTNPVATESYGTVATNHQSAYAQAQTGAVQSQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 401 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGHDNVDADKVMITNEEEIKTTNPVATESYGQVATNKQSAYAQAQTGAVQSQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 402 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAYAQAQTGALQSQGVLPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 403 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNKQSAYAQAQTGAVQSQGVLPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 404 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATEAYGQVATNKQSAYAQAQTGAVQNQGVLPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 405 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNKQSAYAQAQTGALQSQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 406 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGHDNVDADKVMITNEEEIKTTNPVATESYGTVATNHQSAYAQAQTGAVQNQGVLPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 407 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNKQSAYAQAQTGALQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 408 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNKQSAYAQAQTGAVQSQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 409 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAYAQAQTGALQNQGVLPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 410 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGHDNVDADKVMITNEEEIKTTNPVATESYGQVATNHQSAYAQAQTGAVQSQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 411 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATEAYGQVATNHQSAYAQAQTGALQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 412 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGTVATNHQSAYAQAQTGALQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 413 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGTVATNHQSAQAQAQTGWLQSQGVLPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 414 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGHDNVDADKVMITNEEEIKTTNPVATESYGTVATNHQSAYAQAQTGAVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 415 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAYAQAQTGALQSQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 416 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGTVATNKQSAYAQAQTGAVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 417 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGTVATNHQSAYAQAQTGAVQNQGVLPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 418 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGTVATNHQSAYAQAQTGAVQSQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 419 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNKQSAYAQAQTGAVQNQGVLPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 420 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGHDNVDADKVMITNEEEIKTTNPVATEAYGQVATNHQSAYAQAQTGAVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 421 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATEAYGTVATNHQSAYAQAQTGAVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 422 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATEAYGQVATNHQSAYAQAQTGAVQSQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 423 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATEAYGQVATNHQSAYAQAQTGAVQNQGVLPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 424 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATEAYGQVATNKQSAYAQAQTGAVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 425 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGHDNVDADKVMITNEEEIKTTNPVATESYGQVATNHQSAYAQAQTGAVQNQGVLPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 426 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGTVATNHQSAQAQAQTGALQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 427 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAYAQAQTGWLQSQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 428 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATEAYGQVATNHQSAYAQAQTGAVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 429 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAYAQAQTGAVQSQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 430 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGALQSQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 431 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNKQSAYAQAQTGAVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 432 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGHDNVDADKVMITNEEEIKTTNPVATESYGQVATNHQSAYAQAQTGAVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 433 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAYAQAQTGAVQNQGVLPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 434 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAYAQAQTGALQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 435 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGAVQNQGVLPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 436 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAYAQAQTGAVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 437 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGTVATNHQSAQAQAQTGAVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 438 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGALQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 439 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGVLPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 440 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQSQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 441 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWLQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 442 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAYAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 443 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGAVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 444 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGHDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 445 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGTVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

(19) United States
(12) Patent Application Publication
(10) Pub. No.: US 2026/0158170 A1
(43) Pub. Date: Jun. 11, 2026

(54) CAPSID POLYPEPTIDES AND METHODS OF USE THEREOF

(71) Applicant: DYNO THERAPEUTICS, INC., Watertown, MA (US)

(72) Inventors: Samuel WOLOCK, Brookline, MA (US); Megan CRAMER, Medford, MA (US); Elina LOCANE, Cambridge, MA (US); Daniela PIGNATTA, Arlington, MA (US)

(73) Assignee: DYNO THERAPEUTICS, INC., Watertown, MA (US)

(21) Appl. No.: 19/537,081

(22) Filed: Feb. 11, 2026

Related U.S. Application Data
(63) Continuation of application No. PCT/US2025/046858, filed on Sep. 17, 2025.
(60) Provisional application No. 63/695,939, filed on Sep. 18, 2024.

(51) Int. Cl. A61K 48/00; A61K 47/69; C07K 14/005

(52) U.S. Cl. CPC A61K 48/005; A61K 47/6901; C07K 14/005; C12N 2750/14122; C12N 2750/14143

(57) ABSTRACT

The disclosure is directed in part to dependoparvovirus capsid polypeptides that can be used to deliver payloads.

### 1. CROSS-REFERENCE TO RELATED APPLICATIONS

This application is a continuation of international application no. PCT/US2025/046858, filed Sep. 17, 2025, which claims the priority benefit of U.S. provisional application No. 63/695,939, filed Sep. 18, 2024, the contents of each of which are incorporated herein in their entireties by reference thereto.

### 2. SEQUENCE LISTING

The instant application contains a Sequence Listing which has been submitted electronically in XML format and is hereby incorporated by reference in its entirety. Said XML Sequence Listing, created on Sep. 16, 2025, is named DYN-014WO_SL.xml and is 387,426 bytes in size.

### 3. BACKGROUND

Dependoparvoviruses, e.g., adeno-associated dependoparvoviruses, e.g., adeno-associated viruses (AAVs), are of interest as vectors for delivering various payloads to cells, including in human subjects.

### 4. SUMMARY

The present disclosure relates, in part, to improved dependoparvovirus capsid polypeptides, such as VP1, VP2 and/or VP3 capsid polypeptides, methods of producing a dependoparvovirus comprising capsid polypeptides, compositions for use in the same, as well as viral particles produced by the same. In certain aspects, the present disclosure relates to viral particles comprising the improved dependoparvovirus capsid polypeptides, with increased muscle biodistribution and/or transduction as compared to viral particles, e.g., without the mutations in the improved dependoparvovirus capsid polypeptides.

In some aspects, the disclosure provides capsid polypeptides comprising a histidine at a position corresponding to R550 of the VP1 capsid polypeptide of SEQ ID NO:7, an asparagine at a position corresponding to D551 of the VP1 capsid polypeptide of SEQ ID NO:7, an alanine at a position corresponding to S576 of the VP1 capsid polypeptide of SEQ ID NO:7, an isoleucine at a position corresponding to Q579 of the VP1 capsid polypeptide of SEQ ID NO:7, a lysine at a position corresponding to H584 of the VP1 capsid polypeptide of SEQ ID NO:7, an asparagine at a position corresponding to S586 of the VP1 capsid polypeptide of SEQ ID NO:7, a histidine or serine at a position corresponding to A587 of the VP1 capsid polypeptide of SEQ ID NO:7, a tyrosine, a threonine, or asparagine at a position corresponding to Q588 of the VP1 capsid polypeptide of SEQ ID NO:7, a glutamine at a position corresponding to A589 of the VP1 capsid polypeptide of SEQ ID NO:7, an asparagine at a position corresponding to Q592 of the VP1 capsid polypeptide of SEQ ID NO:7, or any combination of two, three, four, five, six, seven, eight, nine, or all ten of the foregoing.

In some embodiments, the disclosure provides capsid polypeptides comprising a histidine at a position corresponding to R550 of the VP1 capsid polypeptide of SEQ ID NO:7, an asparagine at a position corresponding to D551 of the VP1 capsid polypeptide of SEQ ID NO:7, an alanine at a position corresponding to S576 of the VP1 capsid polypeptide of SEQ ID NO:7, a lysine at a position corresponding to H584 of the VP1 capsid polypeptide of SEQ ID NO:7, a tyrosine at a position corresponding to Q588 of the VP1 capsid polypeptide of SEQ ID NO:7, or any combination of two, three, four, or all five of the foregoing.

In some embodiments, the disclosure provides capsid polypeptides comprising a mutation or combination of mutations as described in the preceding two paragraphs (e.g., two, three, four, five mutations described in the preceding paragraph) as compared to a capsid polypeptide of SEQ ID NO:7, together with an alanine at a position corresponding to W595 of the VP1 capsid polypeptide of SEQ ID NO:7, a leucine at a position corresponding to V596 of the VP1 capsid polypeptide of SEQ ID NO:7, a serine at a position corresponding to N598 of the VP1 capsid polypeptide of SEQ ID NO:7, a valine at a position corresponding to 1601 of the VP1 capsid polypeptide of SEQ ID NO:7, or any combination of two, three, or all four of the foregoing.

In some embodiments, the disclosure provides capsid polypeptides comprising a mutation or combination of mutations as described in the preceding three paragraphs as compared to a capsid polypeptide of SEQ ID NO:7, and further comprising a threonine at a position corresponding to Q579 of the VP1 capsid polypeptide of SEQ ID NO:7, a threonine at a position corresponding to A589 of the VP1 capsid polypeptide of SEQ ID NO:7, a valine at a position corresponding to T593 of the VP1 capsid polypeptide of SEQ ID NO:7, or any combination of the foregoing.

In some embodiments, the disclosure provides capsid polypeptides comprising any two or all three of a lysine at a position corresponding to H584 of the VP1 capsid polypeptide of SEQ ID NO:7, a tyrosine at a position corresponding to Q588 of the VP1 capsid polypeptide of SEQ ID NO:7, and an alanine at a position corresponding to W595 of the VP1 capsid polypeptide of SEQ ID NO:7.

In some embodiments, the disclosure provides capsid polypeptides comprising a combination of mutations as described in the preceding paragraph as compared to a capsid polypeptide of SEQ ID NO:7, and further comprising a histidine at a position corresponding to R550 of the VP1 capsid polypeptide of SEQ ID NO:7, an alanine at a position corresponding to S576 of the VP1 capsid polypeptide of SEQ ID NO:7, a threonine at a position corresponding to Q579 of the VP1 capsid polypeptide of SEQ ID NO:7, a leucine at a position corresponding to V596 of the VP1 capsid polypeptide of SEQ ID NO:7, a serine at a position corresponding to N598 of the VP1 capsid polypeptide of SEQ ID NO:7, a valine at a position corresponding to 1601 of the VP1 capsid polypeptide of SEQ ID NO:7, or any combination of two, three, four, five, or all six of the foregoing.

Accordingly, the present disclosure provides a capsid polypeptide described herein. In some embodiments, the capsid polypeptide comprises an amino acid sequence having at least 70%, 75%, 80%, 85%, 90%, 91%, 92%, 93%, 94%, 95%, 96%, 97%, 98%, 99% or 100% identity to a VP1 polypeptide of SEQ ID NO:12 (referred to herein as “V1”), SEQ ID NO:14 (referred to herein as “V2”), SEQ ID NO:16 (referred to herein as “V3”), or SEQ ID NO:18 (referred to herein as “V4”) or to a VP2 or VP3 portion thereof. In some embodiments, a capsid polypeptide of the disclosure comprises one or more mutation differences or a mutation set mutation set present in any one of V1-V4. In some embodiments, the capsid polypeptide comprises an amino acid sequence having at least 70%, 75%, 80%, 85%, 90%, 91%, 92%, 93%, 94%, 95%, 96%, 97%, 98%, 99% or 100% identity to a VP1 polypeptide of any one of SEQ ID NOS:300-445 (referred to herein as “V5” to “V150,” respectively) or to a VP2 or VP3 portion thereof. In some embodiments, a capsid polypeptide of the disclosure comprises one or more mutation differences or a mutation set mutation set present in any one of V5-V150.

In some embodiments, the percentage sequence identity is calculated excluding any targeting peptide sequence insertion(s) in the capsid polypeptide sequence. In other embodiments, the percentage sequence identity is calculated including any targeting peptide sequence insertion(s) in the capsid polypeptide sequence.

Exemplary VP1 amino acid sequences of the disclosure are set forth in SEQ ID NOS:12, 14, 16, and 18. Additional exemplary capsid polypeptides are disclosed in Section 6.2 and numbered embodiments 1 to 326.

The present disclosure further provides a nucleic acid comprising a nucleotide sequence encoding a capsid polypeptide as provided for herein, e.g., a capsid polypeptide disclosed in Section 6.2 or any one of numbered embodiments 1 to 326. In some embodiments, the nucleic acid molecule comprises a nucleotide sequence of SEQ ID NO:13, SEQ ID NO:15, SEQ ID NO:17, or SEQ ID NO:19, a fragment of any of the foregoing (e.g., a fragment thereof encoding a VP2 or VP3 polypeptide), or a variant of any of the foregoing having at least 70%, 75%, 80%, 85%, 90%, 91%, 92%, 93%, 94%, 95%, 96%, 97%, 98%, 99%, or 100% sequence identity thereto. In some embodiments, the percentage sequence identity is calculated excluding any nucleotide sequence(s) encoding targeting peptide sequence insertion(s). In some embodiments, the percentage sequence identity is calculated including any nucleotide sequence(s) encoding targeting peptide sequence insertion(s). In some embodiments, the nucleic acid is a vector, e.g., a plasmid. Exemplary nucleic acids are disclosed in Section 6.2 and numbered embodiments 327 to 397.

The present disclosure further provides a dependoparvovirus particle comprising a capsid polypeptide, a capsid polypeptide disclosed in Section 6.2 or any one of numbered embodiments 1 to 326 and/or a nucleic acid described herein, e.g., a nucleic acid disclosed in Section 6.2 or any one of numbered embodiments 327 to 397 or a nucleic acid comprising a transgene as disclosed in Section 6.7.1. In some embodiments, the dependoparvovirus is an adeno-associated dependoparvovirus (AAV). In some embodiments, the AAV is AAV9, e.g., a variant AAV9. Exemplary virus particles are disclosed in Section 6.4 and numbered embodiments 398 to 429. In some embodiments, the virus particles have one or more characteristics disclosed in Section 6.5 and numbered embodiments 413 to 419.

In some embodiments, the disclosure is directed, in part, to a cell, cell-free system, or other translation system comprising a nucleic acid or vector described herein, e.g., comprising a sequence encoding a capsid polypeptide having one or more mutations described herein, for example a capsid polypeptide disclosed in Section 6.2 or any one of numbered embodiments 1 to 326. In some embodiments, the cell, cell-free system, or other translation system comprises a dependoparvovirus particle described herein, e.g., wherein the particle comprises a nucleic acid comprising a sequence encoding a capsid polypeptide, e.g., a capsid polypeptide disclosed in Section 6.2 or any one of numbered embodiments 1 to 326 and/or a nucleic acid described herein, e.g., a nucleic acid disclosed in Section 6.2 or any one of numbered embodiments 327 to 397 or a nucleic acid comprising a transgene as disclosed in Section 6.7.1. Exemplary cells, cell-free and other translation systems and their use to produce dependoparvovirus particles are disclosed in Section 6.6 and in numbered embodiments 1621 to 1633 and 1637 to 1647.

The present disclosure further provides methods of using a dependoparvovirus disclosed herein, e.g., for delivering a payload to a cell or treating a disease or condition in a subject. The methods typically comprise contacting the cell or administering to the subject a dependoparvovirus particle described herein in an amount effective to treat the disease or condition. Exemplary methods are disclosed in Section 6.7 and numbered embodiments 430 to 1620. The dependoparvovirus particles may be in the form of a composition, e.g., a pharmaceutical composition comprising the dependoparvovirus particles and a pharmaceutically acceptable carrier or excipient, for example as described in Section 6.7 and numbered embodiment 1634. The disclosure further provides compositions disclosed herein for use in treating a disease or condition in a subject and for use in the manufacture of a medicament for use in treating a disease or condition in a subject. Exemplary compositions for use are described in numbered embodiments 1635 and 1636.

Additional features, advantages and applications of the capsid polypeptides, nucleic acids, dependoparvovirus particles of the disclosure and methods of their production and use are more particularly described below.

### 5. BRIEF DESCRIPTION OF THE DRAWINGS

FIGS. 1A-1C. Illustration of exemplary AAV serotype alignments. Amino acids that are present only in VP1 polypeptides are in normal text; amino acids that are present only in VP1 and VP2 polypeptides are in bold; amino acids that are present in VP1, VP2 and VP3 polypeptides are underlined. Figures disclose SEQ ID NOS 3, 1, 7, 5, and 9, respectively, in order of appearance.

FIGS. 2A-2O are graphs of log 2 rate of production, biodistribution, or transduction for variant capsids with single submutations of V1 relative to WT AAV9, where the indicated position has the amino acid corresponding to WT AAV9 and all other positions of the variant contain the mutations of V1.

FIGS. 3A-3O are graphs of log 2 rate of production, biodistribution, or transduction for variant capsids with single submutations of V1 relative to V1, where the indicated position has the amino acid corresponding to WT AAV9 and all other positions of the variant contain the mutations of V1.

FIGS. 4A-4O are graphs of log 2 rate of production, biodistribution, or transduction for variant capsids with submutations of V1 relative to WT AAV9, grouped such that the indicated position has data for variants having the amino acid corresponding to WT AAV9 at that position and any combination of one or more of the mutations of V1.

FIGS. 5A-5O are graphs of log 2 rate of production, biodistribution, or transduction for variant capsids with submutations of V1 relative to V1, grouped such that the indicated position has data for variants having the amino acid corresponding to WT AAV9 at that position and any combination of one or more of the mutations of V1.

FIGS. 6A-6O are graphs of log 2 rate of production, biodistribution, or transduction for variant capsids with submutations of V1 relative to WT AAV9 grouped by the indicated edit distance from variant V1.

### 6. DETAILED DESCRIPTION

### 6.1. Definitions

Unless otherwise defined herein, scientific and technical terms used in connection with the present disclosure shall have the meanings that are commonly understood by those of ordinary skill in the art. Exemplary methods and materials are described below, although methods and materials similar or equivalent to those described herein can also be used in the practice or testing of the present disclosure. In case of conflict, the present specification, including definitions, will control. Generally, nomenclature used in connection with, and techniques of, cell and tissue culture, molecular biology, immunology, microbiology, genetics, analytical chemistry, synthetic organic chemistry, medicinal and pharmaceutical chemistry, and protein and nucleic acid chemistry and hybridization described herein are those well-known and commonly used in the art. Enzymatic reactions and purification techniques are performed according to manufacturer's specifications, as commonly accomplished in the art or as described herein. Further, unless otherwise required by context, singular terms shall include pluralities and plural terms shall include the singular. Throughout this specification and embodiments, the words “have” and “comprise,” or variations such as “has,” “having,” “comprises,” or “comprising,” will be understood to imply the inclusion of a stated integer or group of integers but not the exclusion of any other integer or group of integers. All publications and other references mentioned herein are incorporated by reference in their entirety. Although a number of documents are cited herein, this citation does not constitute an admission that any of these documents forms part of the common general knowledge in the art.

A, An, The: As used herein, the singular forms “a,” “an” and “the” include plural referents unless the context clearly dictates otherwise.

About, Approximately: As used herein, the terms “about” and “approximately” shall generally mean an acceptable degree of error for the quantity measured given the nature or precision of the measurements. Exemplary degrees of error are within 15 percent (%), typically, within 10%, and more typically, within 5% of a given value or range of values. Any disclosure herein of a value preceded by the term “about” or “approximately” is also a disclosure of the value per se. For example, disclosure of “about 10 μg/ml” is a disclosure of the value “10 μg/ml.”

CNS: As used herein, “CNS” means one or more regions of the central nervous system. In some embodiments, the CNS includes one or more of: brain and spinal cord.

Corresponds to: As used herein, the term “corresponds to” as used in reference to a position in a sequence, such as an amino acid or nucleic acid sequence, can be used in reference to an entire capsid polypeptide or polynucleotide sequence, such as the full-length sequence of the capsid polypeptide that comprises a VP1, VP2, and VP3 polypeptide, or a nucleic acid molecule encoding the same. In some embodiments, the term “corresponds to” can be used in reference to a region or domain of the capsid polypeptide. For example, a position that corresponds to a position in the VP1 section of the reference capsid polypeptide can correspond to the VP1 portion of the polypeptide of the variant capsid polypeptide. Thus, when aligning the two sequences to determine whether a position corresponds to another position the full-length polypeptide can be used or domains (regions) can be used to determine whether a position corresponds to a specific position. In some embodiments, the region is the VP1 polypeptide. In some embodiments, the region is the VP2 polypeptide. In some embodiments, the region is the VP3 polypeptide. In some embodiments, when the reference polypeptide is the wild-type sequence (e.g., full-length or region) of a certain serotype of AAV, the variant polypeptide can be of the same serotype with a mutation made at such corresponding position as compared to the reference sequence (e.g., full-length or region). In some embodiments, the variant capsid polypeptide is a different serotype as compared to the reference sequence.

Dependoparvovirus capsid: As used herein, the term “dependoparvovirus capsid” refers to an assembled viral capsid comprising dependoparvovirus polypeptides. In some embodiments, a dependoparvovirus capsid is a functional dependoparvovirus capsid, e.g., is fully folded and/or assembled, is competent to infect a target cell, or remains stable (e.g., folded/assembled and/or competent to infect a target cell) for at least a threshold time.

Dependoparvovirus particle: As used herein, the term “dependoparvovirus particle” refers to an assembled viral capsid comprising dependoparvovirus polypeptides and a packaged nucleic acid, e.g., comprising a payload, one or more components of a dependoparvovirus genome (e.g., a whole dependoparvovirus genome), or both. In some embodiments, a dependoparvovirus particle is a functional dependoparvovirus particle, e.g., comprises a desired payload, is fully folded and/or assembled, is competent to infect a target cell, or remains stable (e.g., folded/assembled and/or competent to infect a target cell) for at least a threshold time.

Dependoparvovirus X particle/capsid: As used herein, the term “dependoparvovirus X particle/capsid” refers to a dependoparvovirus particle/capsid comprising at least one polypeptide or polypeptide encoding nucleic acid sequence derived from a naturally occurring dependoparvovirus X species or serotype. For example, a dependoparvovirus B particle refers to a dependoparvovirus particle comprising at least one polypeptide or polypeptide encoding nucleic acid sequence derived from a naturally occurring dependoparvovirus B sequence. Derived from, as used in this context, means having at least 70, 75, 80, 85, 90, 91, 92, 93, 94, 95, 96, 97, 98, 99, or 100% identity to the sequence in question. Correspondingly, an AAVX particle/capsid, as used herein, refers to an AAV particle/capsid comprising at least one polypeptide or polypeptide encoding nucleic acid sequence derived from a naturally occurring AAV X serotype. For example, an AAV9 particle refers to an AAV particle comprising at least one polypeptide or polypeptide encoding nucleic acid sequence derived from a naturally occurring AAV9 sequence. Sometimes, a dependoparvovirus X capsid is referred to as “Wild Type” or “wt” when such capsid comprises capsid polypeptides from a specified sequence identifier associated with such dependoparvovirus X capsid. Thus, for example, the terms wild-type AAV9 capsid or wtAAV9 capsid (or simply wtAAV9) are used interchangeably and refer to a capsid that comprises capsid polypeptides of SEQ ID NO:7 (e.g., a VP1 capsid of SEQ ID NO:7 and VP2 and VP3 portions thereof).

Edit Distance: Sequences disclosed herein may be described in terms of “edit distance.” The minimum number of sequence edits, i.e., additions, substitutions, or deletions of a single amino acid (for amino acid sequence) or a single nucleotide (for nucleotide sequences), which change one sequence into another sequence is the edit distance between the two sequences. The term “edit distance” is often used interchangeably with the term “Levenshtein distance.”

Exogenous: As used herein, the term “exogenous” refers to a feature, sequence, or component present in a circumstance (e.g., in a nucleic acid, polypeptide, or cell) that does not naturally occur in said circumstance. For example, a nucleic acid sequence encoding a polypeptide can comprise an exogenous codon (e.g., codon encoding for an amino acid that does not naturally occur in that position, for example in a reference sequence), such as provided for herein. Use of the term exogenous in this fashion means that the codon in question at this position does not occur naturally, e.g., is not present in AAV9, e.g., is not present in SEQ ID NO:7. In some embodiments, the codon replaces an endogenous codon. In some embodiments, the exogenous codon is inserted into the nucleic acid sequence, for example, relative to a reference sequence. A person of skill will readily understand that a sequence (e.g., a codon) can be exogenous when provided in a particular sequence (e.g., that does not naturally comprise the codon at the site in question) but may not be exogenous in a second sequence (e.g., that does naturally comprise that particular codon at the site in question).

Functional: As used herein in reference to a polypeptide component of a dependoparvovirus capsid (e.g., Cap (e.g., VP1, VP2, and/or VP3) or Rep), the term “functional” refers to a polypeptide which provides at least 50, 60, 70, 80, 90, or 100% of the activity of a naturally occurring version of that polypeptide component (e.g., when present in a host cell). For example, a functional VP1 polypeptide can stably fold and assemble into a dependoparvovirus capsid (e.g., that is competent for packaging and/or secretion). As used herein in reference to a dependoparvovirus capsid or particle, “functional” refers to a capsid or particle comprising one or more of the following production characteristics: comprises a desired payload, is fully folded and/or assembled, is competent to infect a target cell, or remains stable (e.g., folded/assembled and/or competent to infect a target cell) for at least a threshold time.

Mutation Difference: As used herein with respect to a polypeptide sequence, means a single amino acid mutation (e.g., substitution, insertion or deletion) present in a subject polypeptide sequence, relative to a reference polypeptide sequence. In various embodiments, the reference polypeptide sequence is a polypeptide of any one of SEQ ID NO:1, SEQ ID NO:3, SEQ ID NO:5, SEQ ID NO:7, SEQ ID NO:9, SEQ ID NO:11, SEQ ID NO:12, SEQ ID NO:14, SEQ ID NO: 16, SEQ ID NO:18, or a VP2 or VP3 portion thereof. In some embodiments, the reference polypeptide is any one of SEQ ID NO:7, 12, 14, 16, 18, or a VP2 or VP3 portion thereof. In a preferred embodiment, the reference polypeptide is a polypeptide of SEQ ID NO:7. In various embodiments, the subject polypeptide is SEQ ID NO:12, 14, 16, 18 or a VP2 or VP3 portion thereof.

Mutation Set: As used herein, the term “mutation set” refers to the complete set of single amino acid mutations (substitutions, deletions and/or insertions) in a variant capsid polypeptide sequence (e.g., a polypeptide sequence of SEQ ID NO:12, 14, or a VP2 or VP3 portion thereof) relative to a reference sequence (e.g., a wild-type reference sequence). In some embodiments, the reference sequence is wild-type AAV9 VP1 capsid polypeptide (SEQ ID NO:7) or a VP2 or VP3 portion thereof. In some cases, part of the mutation set (i.e., more than one single amino acid mutation) is notated collectively, however, it will be understood that even when referred to in this way, the mutation set is a collection of single amino acid mutations. For example, an insertion of amino acid 1, 2, and 3 between amino acid N at position nn and amino acid W at position ww of a reference sequence may be notated as “Nnn_3aa_Www_123,” and it will be understood that each of amino acids 1, 2 and 3 represent separate single amino acid mutations within the mutation set. The mutation sets for certain variant capsid polypeptides described herein are found, for example, in Table 1. In some embodiments, a variant capsid polypeptide of the disclosure comprises a mutation set not consisting solely of a mutation set present in a capsid polypeptide of SEQ ID NO:12, 14, 16, or 18.

Nucleic Acid: As used herein, in its broadest sense, the term “nucleic acid” refers to any compound and/or substance that is or can be incorporated into an oligonucleotide chain. In some embodiments, a nucleic acid is a compound and/or substance that is or can be incorporated into an oligonucleotide chain via a phosphodiester linkage. As will be clear from context, in some embodiments, “nucleic acid” refers to an individual nucleic acid monomer (e.g., a nucleotide and/or nucleoside); in some embodiments, “nucleic acid” refers to an oligonucleotide chain comprising individual nucleic acid monomers or a longer polynucleotide chain comprising many individual nucleic acid monomers. In some embodiments, a “nucleic acid” is or comprises RNA; in some embodiments, a “nucleic acid” is or comprises DNA. In some embodiments, a nucleic acid is, comprises, or consists of one or more natural nucleic acid residues. In some embodiments, a nucleic acid is, comprises, or consists of one or more nucleic acid analogs. In some embodiments, a nucleic acid is, comprises, or consists of one or more modified, synthetic, or non-naturally occurring nucleotides. In some embodiments, a nucleic acid analog differs from a nucleic acid in that it does not utilize a phosphodiester backbone. For example, in some embodiments, a nucleic acid is, comprises, or consists of one or more “peptide nucleic acids”, which are known in the art and have peptide bonds instead of phosphodiester bonds in the backbone, are considered within the scope of the present invention. Alternatively or additionally, in some embodiments, a nucleic acid has one or more phosphorothioate and/or 5′-N-phosphoramidite linkages rather than phosphodiester bonds. In some embodiments, a nucleic acid has a nucleotide sequence that encodes a functional gene product such as an RNA or protein. In some embodiments, a nucleic acid is partly or wholly single stranded; in some embodiments, a nucleic acid is partly or wholly double stranded.

Or: Unless indicated otherwise, an “or” conjunction is intended to be used in its correct sense as a Boolean logical operator, encompassing both the selection of features in the alternative (A or B, where the selection of A is mutually exclusive from B) and the selection of features in conjunction (A or B, where both A and B are selected). In some places in the text, the term “and/or” is used for the same purpose, which shall not be construed to imply that “or” is used with reference to mutually exclusive alternatives.

Percent Identity: Sequences disclosed herein may be described in terms of “percent identity” (% identity). For calculating percent identity between two amino acid sequences or two nucleic acid sequences, the two sequences to be compared are aligned using the EMBOSS Needle Pairwise Sequence Alignment software tool based on the Needleman and Wunsch algorithm (Needleman & Wunsch, 1970, J. Mol. Biol. 48(3):443-53) (available at www.ebi.ac.uk/Tools/psa/emboss_needle/) using the following parameters: Matrix: BLOSUM62 (for amino acid sequences) or DNAfull (for DNA sequences); Gap Open: 10; Gap Extend: 0.5; End Gap Penalty: false; End Gap Open: 10; and End Gap Extend: 0.5. Percent identity is determined by dividing the number of amino acid or nucleotide matches in the alignment by the length of the alignment and multiplying by 100. For example, if an alignment of two amino acid sequences has 95 matching amino acids and an alignment length of 100 amino acids, the two sequences have 95% identity.

When calculating percent identity of two capsid polypeptides, one or both of which contain(s) one or more targeting peptide insertions, percent identity can be determined without removing the targeting peptide insertion sequence(s) from the capsid polypeptide sequence(s) or, alternatively, percent identity can be determined after removing the targeting peptide insertion sequence(s) from the capsid polypeptide sequence(s). For example, if a first capsid polypeptide has an identical sequence to a second capsid polypeptide, except that the first capsid polypeptide has a 7-mer targeting peptide insertion, the two capsid polypeptides have less than 100% sequence identity when percent identity is determined without removal of the targeting peptide insertion sequence from the first capsid polypeptide sequence, whereas the two capsid polypeptides have 100% sequence identity when the targeting peptide insertion sequence is removed from the first capsid polypeptide sequence prior to calculating percent identity. References herein to percent identity of capsid polypeptides without mention of a targeting peptide refer to percent identity of the capsid polypeptides determined following removal of targeting polypeptide insertion sequence(s), if any, present in both capsid polypeptides, unless required otherwise by context. References herein to percent identity calculated “taking targeting peptide insertions into account” means that the percent identity is calculated without removal of targeting polypeptide insertion sequence(s), if any, present in both capsid polypeptides. References herein to percent identity calculated “without taking targeting peptide insertions into account” means that the percent identity is calculated following removal of targeting polypeptide insertion sequence(s), if any, present in both capsid polypeptides.

PNS: as used herein, “PNS” means one or more regions of the peripheral nervous system that does not include the CNS. In some embodiments, the PNS includes dorsal root ganglia. In some embodiments, the PNS includes sensory neurons and motor neurons.

Polypeptide, peptide, and protein: The terms “polypeptide,” “peptide” and “protein” are used interchangeably herein to refer to polymers of amino acids of any length. The polymer may be linear or branched, it may comprise modified amino acids, and it may be interrupted by non-amino acids.

Targeting Peptide: As used herein, a “targeting peptide” refers to a peptide inserted into, or attached to, a capsid polypeptide to alter the tropism of the capsid polypeptide. A targeting peptide can be inserted into an AAV capsid sequence for enhanced targeting to a desired cell-type, tissue, or organ, for example for enhanced targeting to the CNS or to muscle tissue. A targeting peptide is typically 3 to 20 amino acids in length, for example, 3 to 12 amino acids, 5 to 12 amino acids, 5 to 10 amino acids, or 7 to 10 amino acids in length.

Treating: As used herein, the term “treating a disease or condition” refers to treating a manifest disease or condition, for example, where the subject is already suffering from one or more symptoms of the disease or condition, or refers to treating a pre-manifest disease or condition, for example, where the subject is identified as having a disease or condition but is not yet exhibiting one or more symptoms of the disease or condition. Pre-manifest conditions may be identified by, for example, genetic testing.

Variant: As used herein, a “variant capsid polypeptide” refers to a polypeptide that differs from a reference sequence (e.g., SEQ ID NO:1, SEQ ID NO:3, SEQ ID NO:5, SEQ ID NO:7, SEQ ID NO:9, or SEQ ID NO:11, preferably SEQ ID NO:7), or sequence subunit thereof such as a VP2 or VP3 portion thereof). The variant capsid polypeptide can, for example, comprise a mutation (e.g., substitution, deletion, or insertion). In some embodiments, the variant is about, or at least, 70%, 75%, 80%, 85%, 86%, 87%, 88%, 89%, 90%, 91%, 92%, 93%, 94%, 95%, 96%, 97%, 98%, or 99% identical to the reference sequence. It will be clear to the skilled artisan from this disclosure that any capsid polypeptide, for example any capsid polypeptide disclosed herein, for example, a capsid polypeptide of SEQ ID NO:12, 14, 16, or 18, is a variant capsid polypeptide with respect to another capsid polypeptide having a different amino acid sequence, e.g., another capsid polypeptide with a reference sequence as set forth above. Thus, the term “variant capsid polypeptide” herein means, and is used interchangeably with, “capsid polypeptide,” and does not require any comparison to a specific reference sequence. In some embodiments, the reference sequence is a polypeptide comprising SEQ ID NO:7. In some embodiments, the reference sequence comprises or consists of a VP1, VP2 or VP3 polypeptide, e.g., of SEQ ID NO:7.

In some contexts used herein, the term “variant” refers to a virus particle that includes a variant capsid polypeptide, e.g., described herein.

### 6.2. Capsid Polypeptides

The disclosure is directed, in part, to a variant capsid polypeptide, wherein the variant capsid polypeptide comprises a mutation (insertion, deletion, or substitution) as compared to the wild-type sequence. In some embodiments, the wild-type sequence is SEQ ID NO:7. The disclosure is directed, in part, to a variant capsid polypeptide comprising SEQ ID NO:7 with one or more mutations as compared to SEQ ID NO:7. The mutation can be, for example, an insertion, deletion, or substitution as compared to the wild-type sequence. In some embodiments, the wild-type sequence is SEQ ID NO:7.

In some embodiments, disclosed are variant capsid polypeptides that increase muscle (cardiac and/or skeletal muscle) biodistribution and/or transduction, and/or that increase CNS (e.g., brain) biodistribution and/or transduction when incorporated into a viral particle as compared to reference capsid polypeptides, e.g., wild-type capsid polypeptides. The variant capsid polypeptides comprise a mutation or mutation set as compared to a reference capsid polypeptide, e.g., a VP1 capsid polypeptide of SEQ ID NO:7 or a VP2 or VP3 portion thereof.

In some embodiments, the disclosure provides a variant capsid polypeptide that comprises one or more mutation differences associated with the variant capsid polypeptide V1 (corresponding to a capsid polypeptide of SEQ ID NO:12), V2 (corresponding to a capsid polypeptide of SEQ ID NO:14), V3 (corresponding to a capsid polypeptide of SEQ ID NO:16), or V4 (corresponding to a capsid polypeptide of SEQ ID NO:18). In some embodiments, the disclosure provides a variant capsid polypeptide that comprises one or more mutation differences associated with any one of V5-V150 (V5-V150 correspond to capsid polypeptides of SEQ ID NOS:300-450, respectively).

Mutations associated with V1, V2, V3, and V4 are shown in Table 1 in relation to a VP1 polypeptide of SEQ ID NO:7. V5-V150 comprise one or more mutations associated with V1.

TABLE 1

| | | | | | | | | | | | | | | | | Edit |
|---|---|---|---|---|---|---|---|---|---|---|---|---|---|---|---|---|
| Capsid | | | | | | | | | | | | | | | | distance |
| Poly- | | | | | | | | | | | | | | | | to SEQ |
| peptide | R550 | D551 | S576 | Q579 | H584 | S586 | A587 | Q588 | A589 | Q592 | T593 | W595 | V596 | N598 | I601 | ID NO: 7 |
| V1 | H | | A | T | K | | | Y | | | | A | L | S | V | 9 |
| V2 | H | N | A | T | K | | | Y | | | | A | L | S | V | 10 |
| V3 | H | | | T | K | N | H | T | Q | | | A | L | S | V | 11 |
| V4 | | | | I | | | S | N | T | N | V | A | L | S | V | 10 |

Accordingly, in some embodiments, the disclosure provides a variant capsid polypeptide that comprises one or more of the mutation differences or the entire mutation set associated with V1. In other embodiments, the disclosure provides a variant capsid polypeptide that comprises one or more of the mutation differences or the entire mutation set associated with V2. In other embodiments, the disclosure provides a variant capsid polypeptide that comprises one or more of the mutation differences or the entire mutation set associated with V3. In other embodiments, the disclosure provides a variant capsid polypeptide that comprises one or more of the mutation differences or the entire mutation set associated with V4.

Typically, the variant capsid polypeptide comprises one or more mutation differences or the entire mutation set associated with V1, V2, V3, or V4 and has at least 80%, at least 85%, at least 90%, at least 91%, at least 92%, at least 93%, at least 94%, at least 95%, at least 96%, at least 97%, at least 98%, or at least 99% sequence identity to a reference AAV serotype, e.g., as described herein, e.g., to SEQ ID NO:7. In some embodiments, the variant capsid polypeptide comprises one or more mutation differences or the entire mutation set associated with any one of V5-V150 and has at least 80%, at least 85%, at least 90%, at least 91%, at least 92%, at least 93%, at least 94%, at least 95%, at least 96%, at least 97%, at least 98%, or at least 99% sequence identity to a reference AAV serotype, e.g., as described herein, e.g., to SEQ ID NO:7.

In various embodiments, the variant capsid polypeptide is, but for the mutation differences or mutation set associated with V1, V2, V3, or V4, at least 90%, at least 95%, 96%, 97%, 98%, 99%, or 100% identical to a reference AAV serotype described herein. In various embodiments, the variant capsid polypeptide is, but for the mutation differences or mutation set associated with V1, V2, V3, or V4, at least 90%, at least 95%, 96%, 97%, 98%, 99%, or 100% identical to a capsid polypeptide of SEQ ID NO:1 (e.g., a VP1, VP2 or VP3 sequence of SEQ ID NO: 1). In various embodiments, the variant capsid polypeptide is, but for the mutation differences or mutation set associated with any one of V5-V150, at least 90%, at least 95%, 96%, 97%, 98%, 99%, or 100% identical to a reference AAV serotype described herein. In various embodiments, the variant capsid polypeptide is, but for the mutation differences or mutation set associated with any one of V5-V150, at least 90%, at least 95%, 96%, 97%, 98%, 99%, or 100% identical to a capsid polypeptide of SEQ ID NO:1 (e.g., a VP1, VP2 or VP3 sequence of SEQ ID NO: 1).

In various embodiments, the variant capsid polypeptide is, but for the mutation differences or mutation set associated with V1, V2, V3, or V4, at least 90%, at least 95%, 96%, 97%, 98%, 99%, or 100% identical to a capsid polypeptide of SEQ ID NO:3 (e.g., a VP1, VP2 or VP3 sequence of SEQ ID NO:3). In various embodiments, the variant capsid polypeptide is, but for the mutation differences or mutation set associated with any one of V5-V150, at least 90%, at least 95%, 96%, 97%, 98%, 99%, or 100% identical to a capsid polypeptide of SEQ ID NO:3 (e.g., a VP1, VP2 or VP3 sequence of SEQ ID NO:3).

In various embodiments, the variant capsid polypeptide is, but for the mutation differences or mutation set associated with V1, V2, V3, or V4, at least 90%, at least 95%, 96%, 97%, 98%, 99%, or 100% identical to a capsid polypeptide of SEQ ID NO:5 (e.g., a VP1, VP2 or VP3 sequence of SEQ ID NO:5). In various embodiments, the variant capsid polypeptide is, but for the mutation differences or mutation set associated with any one of V5-V150, at least 90%, at least 95%, 96%, 97%, 98%, 99%, or 100% identical to a capsid polypeptide of SEQ ID NO:5 (e.g., a VP1, VP2 or VP3 sequence of SEQ ID NO:5).

In various embodiments, the variant capsid polypeptide is, but for the mutation differences or mutation set associated with V1, V2, V3, or V4, at least 90%, at least 95%, 96%, 97%, 98%, 99%, or 100% identical to a capsid polypeptide of SEQ ID NO:7 (e.g., a VP1, VP2 or VP3 sequence of SEQ ID NO:7). In various embodiments, the variant capsid polypeptide is, but for the mutation differences or mutation set associated with any one of V5-V150, at least 90%, at least 95%, 96%, 97%, 98%, 99%, or 100% identical to a capsid polypeptide of SEQ ID NO:7 (e.g., a VP1, VP2 or VP3 sequence of SEQ ID NO:7).

In various embodiments, the variant capsid polypeptide is, but for the mutation differences or mutation set associated with V1, V2, V3, or V4, at least 90%, at least 95%, 96%, 97%, 98%, 99%, or 100% identical to a capsid polypeptide of SEQ ID NO:9 (e.g., a VP1, VP2 or VP3 sequence of SEQ ID NO:9). In various embodiments, the variant capsid polypeptide is, but for the mutation differences or mutation set associated with any one of V5-V150, at least 90%, at least 95%, 96%, 97%, 98%, 99%, or 100% identical to a capsid polypeptide of SEQ ID NO:9 (e.g., a VP1, VP2 or VP3 sequence of SEQ ID NO:9).

In various embodiments, the variant capsid polypeptide is, but for the mutation differences or mutation set associated with V1, V2, V3, or V4, at least 90%, at least 95%, 96%, 97%, 98%, 99%, or 100% identical to a capsid polypeptide of SEQ ID NO:11 (e.g., a VP1, VP2 or VP3 sequence of SEQ ID NO:11). In various embodiments, the variant capsid polypeptide is, but for the mutation differences or mutation set associated with any one of V5-V150, at least 90%, at least 95%, 96%, 97%, 98%, 99%, or 100% identical to a capsid polypeptide of SEQ ID NO:11 (e.g., a VP1, VP2 or VP3 sequence of SEQ ID NO:11).

In some embodiments, a variant capsid polypeptide is provided that comprises a variant capsid polypeptide that is at least 70%, 75%, 80%, 85%, 90%, 91%, 92%, 93%, 94%, 95%, 96%, 97%, 98%, 99%, or 100% identical to a variant capsid polypeptide as provided herein, e.g., a VP1 capsid polypeptide of SEQ ID NO: 12, SEQ ID NO: 14, SEQ ID NO:16, or SEQ ID NO:18, or a VP2 or VP3 portion thereof. In some embodiments, a variant capsid polypeptide is provided that comprises a variant capsid polypeptide that is at least 70%, 75%, 80%, 85%, 90%, 91%, 92%, 93%, 94%, 95%, 96%, 97%, 98%, 99%, or 100% identical to a variant capsid polypeptide of any one of SEQ ID NOS:300-445, e.g., a VP1 capsid polypeptide of any one of SEQ ID NOs:300-445, or a VP2 or VP3 portion thereof.

In some embodiments, the variant capsid polypeptide comprises a VP1, VP2, VP3, or any combination thereof, that each has about 1 to about 20 mutations as compared to a VP1 polypeptide of SEQ ID NO: 12, 14, 16, or 18, and comprises one or more of the mutation differences or the entire mutation set of V1, V2, V3, or V4. In some embodiments, the variant capsid polypeptide comprises a VP1, VP2, VP3, or any combination thereof, that each has about 1 to about 20 mutations as compared to a VP1 polypeptide of any one of SEQ ID NOS:300-445, and comprises one or more of the mutation differences or the entire mutation set of the any one of SEQ ID NOS:300-445.

In some embodiments, the variant capsid polypeptide comprises a VP1, VP2, VP3, or any combination thereof, that each has about 1 to about 10 mutations as compared to a VP1 polypeptide of SEQ ID NO: 12, 14, 16, or 18, and comprises one or more of the mutation differences or the entire mutation set of V1, V2, V3, or V4. In some embodiments, the variant capsid polypeptide comprises a VP1, VP2, VP3, or any combination thereof, that each has about 1 to about 10 mutations as compared to a VP1 polypeptide of any one of SEQ ID NOS:300-445, and comprises one or more of the mutation differences or the entire mutation set of the any one of SEQ ID NOS:300-445.

In some embodiments, the variant capsid polypeptide comprises a VP1, VP2, VP3, or any combination thereof, that each has about 1 to about 5 mutations as compared to a VP1 polypeptide of SEQ ID NO: 12, 14, 16, or 18, and comprises one or more of the mutation differences or the entire mutation set of V1, V2, V3, or V4. In some embodiments, the variant capsid polypeptide comprises a VP1, VP2, VP3, or any combination thereof, that each has about 1 to about 5 mutations as compared to a VP1 polypeptide of any one of SEQ ID NOS:300-445, and comprises one or more of the mutation differences or the entire mutation set of the any one of SEQ ID NOS:300-445.

The mutations to capsid polypeptide sequences described herein are described in relation to a position and/or amino acid at a position within a reference sequence, e.g., SEQ ID NO:7. Thus, in some embodiments, the capsid polypeptides described herein are variant capsid polypeptides of the reference sequence, e.g., SEQ ID NO:7, e.g., include capsid polypeptides comprising at least 80%, at least 85%, at least 90%, at least 91%, at least 92%, at least 93%, at least 94%, at least 95%, at least 96%, at least 97%, at least 98%, or at least 99% identity to the reference capsid polypeptide sequence (e.g., reference capsid polypeptide VP1, VP2 and/or VP3 sequence), e.g., SEQ ID NO:7 (or VP2 or VP3 sequence comprised therein) and include one or more mutations described herein.

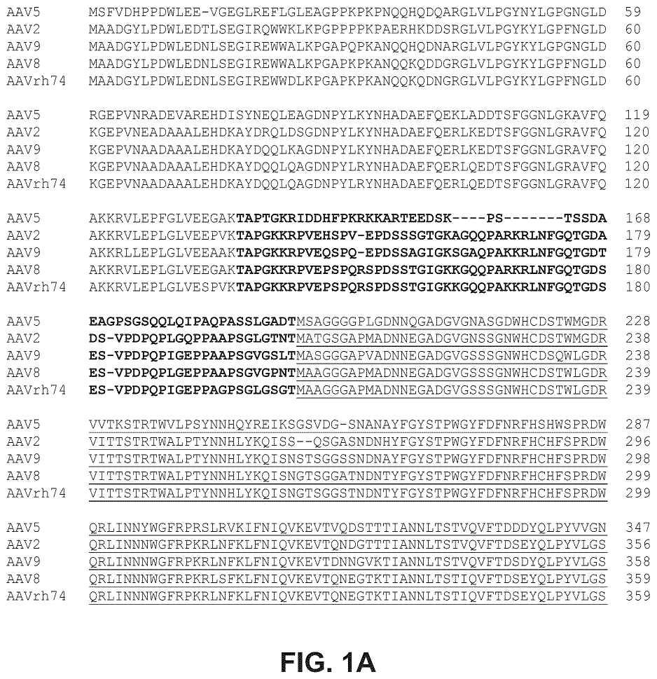

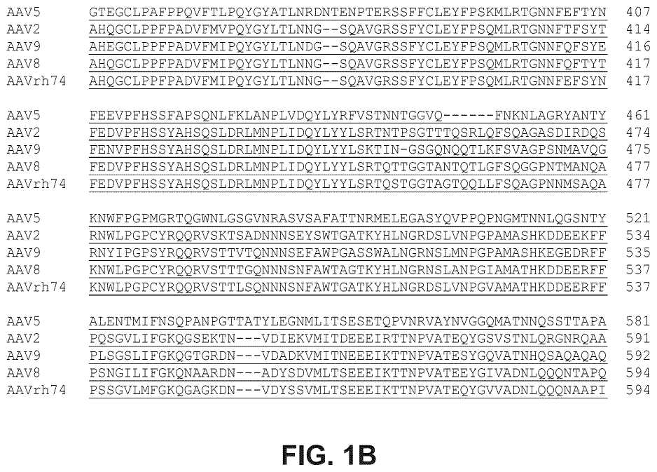

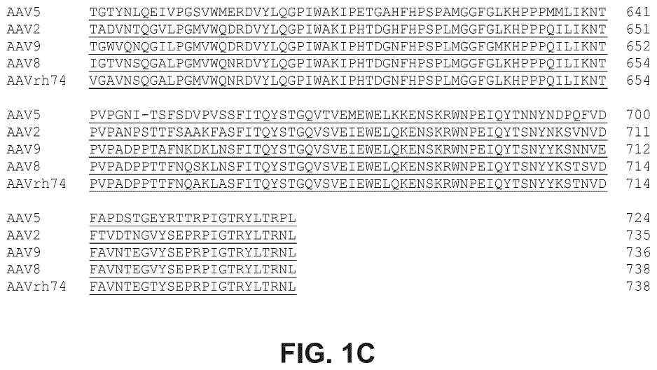

It will be understood by the skilled artisan, and without being bound by theory, that each amino acid position within a reference sequence corresponds to a position within the sequence of other reference capsid polypeptides such as capsid polypeptides derived from dependoparvoviruses with different serotypes. Such corresponding positions are identified using sequence alignment tools known in the art. A particularly preferred sequence alignment tool is EMBOSS Needle Pairwise Sequence Alignment software tool based on the Needleman and Wunsch algorithm (Needleman & Wunsch, 1970, J. Mol. Biol. 48(3):443-53) (available on the World Wide Web at ebi.ac.uk/Tools/psa/emboss_needle/). An alignment of exemplary reference capsid polypeptides is shown in FIGS. 1A-1C. Thus, in some embodiments, the variant capsid polypeptides of the invention include variants of reference capsid polypeptides that include one or more mutations described herein in such reference capsid polypeptides at positions corresponding to the position of the mutation described herein in relation to a different reference capsid polypeptide. Thus, for example, a mutation described as XnnnY relative to SEQ ID NO:7 (where X is the amino acid present at position nnn in SEQ ID NO:7 and Y is the amino acid mutation at that position, e.g., described herein), the disclosure provides variant capsid polypeptides comprising at least 70%, at least 75%, at least 80%, at least 85%, at least 90%, at least 91%, at least 92%, at least 93%, at least 94%, at least 95%, at least 96%, at least 97%, at least 98%, or at least 99% identity to a reference capsid polypeptide sequence (e.g., reference capsid polypeptide VP1, VP2 and/or VP3 sequence) other than SEQ ID NO:7 (or VP2 or VP3 sequence comprised therein) and further comprising the disclosed mutation at a position corresponding to position nnn of SEQ ID NO:7 (e.g., comprising Y at the position in the new variant capsid polypeptide sequence that corresponds to position nnn of SEQ ID NO:7). As described above, such corresponding position is determined using a sequence alignment tool, such as, for example, the clustal omega tool described above. Examples of corresponding amino acid positions of exemplary known AAV serotypes is provided in FIGS. 1A-1C. In some embodiments, the variant is a variant of the AAV9 capsid polypeptide, which can be referred to as a “AAV9 variant capsid polypeptide” or “variant AAV9 capsid polypeptide.”

Thus, in some embodiments, the disclosure provides variant capsid polypeptide sequences that are variants of a reference sequence other than SEQ ID NO:7, e.g., a reference sequence other than SEQ ID NO:7 as described herein, which include one or more mutation corresponding to the mutations described herein. In some embodiments, such variants include mutations corresponding to all of the mutations associated with SEQ ID NO:12. In some embodiments, such variants include mutations corresponding to all of the mutations associated with SEQ ID NO:14. In some embodiments, such variants include mutations corresponding to all of the mutations associated with SEQ ID NO:16. In some embodiments, such variants include mutations corresponding to all of the mutations associated with SEQ ID NO:18. In some embodiments, such variants include mutations corresponding to all of the mutations associated with any one of SEQ ID NOS:300-445.

The variant capsid polypeptides described herein are optionally variants of reference capsids serotypes known in the art. Non-limiting examples of such reference AAV serotypes include AAV1, AAVrh10, AAV-DJ, AAV-DJ8, AAV5, AAVPHP.B (PHP.B), AAVPHP.A (PHP.A), AAVG2B-26, AAVG2B-13, AAVTH1.1-32, AAVTH1.1-35, AAVPHP.B2 (PHP.B2), AAVPHP.B3 (PHP.B3), AAVPHP.N/PHP.B-DGT, AAVPHP.B-EST, AAVPHP.B-GGT, AAVPHP.B-ATP, AAVPHP.B-ATT-T, AAVPHP.B-DGT-T, AAVPHP.B-GGT-T, AAVPHP.B-SGS, AAVPHP.B-AQP, AAVPHP.B-QQP, AAVPHP.B-SNP(3), AAVPHP.B-SNP, AAVPHP.B-QGT, AAVPHP.B-NQT, AAVPHP.B-EGS, AAVPHP.B-SGN, AAVPHP.B-EGT, AAVPHP.B-DST, AAVPHP.B-DST, AAVPHP.B-STP, AAVPHP.B-PQP, AAVPHP.B-SQP, AAVPHP.B-QLP, AAVPHP.B-TMP, AAVPHP.B-TTP, AAVPHP.eB, AAVPHP.S/G2A12, AAVG2A15/G2A3 (G2A3), AAVG2B4 (G2B4), AAVG2B5 (G2B5), PHP.S, AAV2, AAV2G9, AAV3, AAV3a, AAV3b, AAV3-3, AAV4, AAV4-4, AAV6, AAV6.1, AAV6.2, AAV6.1.2, AAV7, AAV7.2, AAV8, AAV9.11, AAV9.13, AAV9, AAV9 K449R (or K449R AAV9), AAV9.16, AAV9.24, AAV9.45, AAbiodisV9.47, AAV9.61, AAV9.68, AAV9.84, AAV9.9, AAV10, AAV11, AAV12, AAV16.3, AAV24.1, AAV27.3, AAV42.12, AAV42-1b, AAV42-2, AAV42-3a, AAV42-3b, AAV42-4, AAV42-5a, AAV42-5b, AAV42-6b, AAV42-8, AAV42-10, AAV42-11, AAV42-12, AAV42-13, AAV42-15, AAV42-aa, AAV43-1, AAV43-12, AAV43-20, AAV43-21, AAV43-23, AAV43-25, AAV43-5, AAV44.1, AAV44.2, AAV44.5, AAV223.1, AAV223.2, AAV223.4, AAV223.5, AAV223.6, AAV223.7, AAV1-7/rh.48, AAV1-8/rh.49, AAV2-15/rh.62, AAV2-3/rh.61, AAV2-4/rh.50, AAV2-5/rh.51, AAV3.1/hu.6, AAV3.1/hu.9, AAV3-9/rh.52, AAV3-11/rh.53, AAV4-8/r11.64, AAV4-9/rh.54, AAV4-19/rh.55, AAV5-3/rh.57, AAV5-22/rh.58, AAV7.3/hu.7, AAV16.8/hu.10, AAV16.12/hu.11, AAV29.3/bb.1, AAV29.5/bb.2, AAV106.1/hu.37, AAV114.3/hu.40, AAV127.2/hu.41, AAV127.5/hu.42, AAV128.3/hu.44, AAV130.4/hu.48, AAV145.1/hu.53, AAV145.5/hu.54, AAV145.6/hu.55, AAV161.10/hu.60, AAV161.6/hu.61, AAV33.12/hu.17, AAV33.4/hu.15, AAV33.8/hu.16, AAV52/hu.19, AAV52.1/hu.20, AAV58.2/hu.25, AAVA3.3, AAVA3.4, AAVA3.5, AAVA3.7, AAVC1, AAVC2, AAVC5, AAVF3, AAVF5, AAVH2, AAVrh.72, AAVhu.8, AAVrh.68, AAVrh.70, AAVpi.1, AAVpi.3, AAVpi.2, AAVrh.60, AAVrh.44, AAVrh.65, AAVrh.55, AAVrh.47, AAVrh.69, AAVrh.45, AAVrh.59, AAVhu.12, AAVH6, AAVH-1/hu.1, AAVH-5/hu.3, AAVLG-10/rh.40, AAVLG-4/rh.38, AAVLG-9/hu.39, AAVN721-8/rh.43, AAVCh.5, AAVCh.5R1, AAVcy.2, AAVcy.3, AAVcy.4, AAVcy.5, AAVCy.5R1, AAVCy.5R2, AAVCy.5R3, AAVCy.5R4, AAVcy.6, AAVhu.1, AAVhu.2, AAVhu.3, AAVhu.4, AAVhu.5, AAVhu.6, AAVhu.7, AAVhu.9, AAVhu.10, AAVhu.11, AAVhu.13, AAVhu.15, AAVhu.16, AAVhu.17, AAVhu.18, AAVhu.20, AAVhu.21, AAVhu.22, AAVhu.23.2, AAVhu.24, AAVhu.25, AAVhu.27, AAVhu.28, AAVhu.29, AAVhu.29R, AAVhu.31, AAVhu.32, AAVhu.34, AAVhu.35, AAVhu.37, AAVhu.39, AAVhu.40, AAVhu.41, AAVhu.42, AAVhu.43, AAVhu.44, AAVhu.44R1, AAVhu.44R2, AAVhu.44R3, AAVhu.45, AAVhu.46, AAVhu.47, AAVhu.48, AAVhu.48R1, AAVhu.48R2, AAVhu.48R3, AAVhu.49, AAVhu.51, AAVhu.52, AAVhu.54, AAVhu.55, AAVhu.56, AAVhu.57, AAVhu.58, AAVhu.60, AAVhu.61, AAVhu.63, AAVhu.64, AAVhu.66, AAVhu.67, AAVhu.14/9, AAVhu.t 19, AAVrh.2, AAVrh.2R, AAVrh.8, AAVrh.8R, AAVrh.10, AAVrh.12, AAVrh.13, AAVrh.13R, AAVrh.14, AAVrh.17, AAVrh.18, AAVrh.19, AAVrh.20, AAVrh.21, AAVrh.22, AAVrh.23, AAVrh.24, AAVrh.25, AAVrh.31, AAVrh.32, AAVrh.33, AAVrh.34, AAVrh.35, AAVrh.36, AAVrh.37, AAVrh.37R2, AAVrh.38, AAVrh.39, AAVrh.40, AAVrh.46, AAVrh.48, AAVrh.48.1, AAVrh.48.1.2, AAVrh.48.2, AAVrh.49, AAVrh.51, AAVrh.52, AAVrh.53, AAVrh.54, AAVrh.56, AAVrh.57, AAVrh.58, AAVrh.61, AAVrh.64, AAVrh.64R1, AAVrh.64R2, AAVrh.67, AAVrh.73, AAVrh.74 (also referred to as AAVrh74), AAVrh8R, AAVrh8R A586R mutant, AAVrh8R R533A mutant, AAAV, BAAV, caprine AAV, bovine AAV, AAVhE1.1, AAVhEr1.5, AAVhER1.14, AAVhEr1.8, AAVhEr1.16, AAVhEr1.18, AAVhEr1.35, AAVhEr1.7, AAVhEr1.36, AAVhEr2.29, AAVhEr2.4, AAVhEr2.16, AAVhEr2.30, AAVhEr2.31, AAVhEr2.36, AAVhER1.23, AAVhEr3.1, AAV2.5T, AAV-PAEC, AAV-LK01, AAV-LK02, AAV-LK03, AAV-LK04, AAV-LK05, AAV-LK06, AAV-LK07, AAV-LK08, AAV-LK09, AAV-LK10, AAV-LK11, AAV-LK12, AAV-LK13, AAV-LK14, AAV-LK15, AAV-LK16, AAV-LK17, AAV-LK18, AAV-LK19, AAV-PAEC2, AAV-PAEC4, AAV-PAEC6, AAV-PAEC7, AAV-PAEC8, AAV-PAEC11, AAV-PAEC12, AAV-2-pre-miRNA-101, AAV-8h, AAV-8b, AAV-h, AAV-b, AAV SM 10-2, AAV Shuffle 100-1, AAV Shuffle 100-3, AAV Shuffle 100-7, AAV Shuffle 10-2, AAV Shuffle 10-6, AAV Shuffle 10-8, AAV Shuffle 100-2, AAV SM 10-1, AAV SM 10-8, AAV SM 100-3, AAV SM 100-10, BNP61 AAV, BNP62 AAV, BNP63 AAV, AAVrh.50, AAVrh.43, AAVrh.62, AAVrh.48, AAVhu.19, AAVhu.11, AAVhu.53, AAV4-8/rh.64, AAVLG-9/hu.39, AAV54.5/hu.23, AAV54.2/hu.22, AAV54.7/hu.24, AAV54.1/hu.21, AAV54.4R/hu.27, AAV46.2/hu.28, AAV46.6/hu.29, AAV128.1/hu.43, true type AAV (ttAAV), UPENN AAV 10, Japanese AAV 10 serotypes, AAV CBr-7.1, AAV CBr-7.10, AAV CBr-7.2, AAV CBr-7.3, AAV CBr-7.4, AAV CBr-7.5, AAV CBr-7.7, AAV CBr-7.8, AAV CBr-B7.3, AAV CBr-B7.4, AAV CBr-E1, AAV CBr-E2, AAV CBr-E3, AAV CBr-E4, AAV CBr-E5, AAV CBr-e5, AAV CBr-E6, AAV CBr-E7, AAV CBr-E8, AAV CHt-1, AAV CHt-2, AAV CHt-3, AAV CHt-6.1, AAV CHt-6.10, AAV CHt-6.5, AAV CHt-6.6, AAV CHt-6.7, AAV CHt-6.8, AAV CHt-P1, AAV CHt-P2, AAV CHt-P5, AAV CHt-P6, AAV CHt-P8, AAV CHt-P9, AAV CKd-1, AAV CKd-10, AAV CKd-2, AAV CKd-3, AAV CKd-4, AAV CKd-6, AAV CKd-7, AAV CKd-8, AAV CKd-B1, AAV CKd-B2, AAV CKd-B3, AAV CKd-B4, AAV CKd-B5, AAV CKd-B6, AAV CKd-B7, AAV CKd-B8, AAV CKd-H1, AAV CKd-H2, AAV CKd-H3, AAV CKd-H4, AAV CKd-H5, AAV CKd-H6, AAV CKd-N3, AAV CKd-N4, AAV CKd-N9, AAV CLg-F1, AAV CLg-F2, AAV CLg-F3, AAV CLg-F4, AAV CLg-F5, AAV CLg-F6, AAV CLg-F7, AAV CLg-F8, AAV CLv-1, AAV CLv1-1, AAV CIv1-10, AAV CLv1-2, AAV CLv-12, AAV CLv1-3, AAV CLv-13, AAV CLv1-4, AAV Clv1-7, AAV Clv1-8, AAV Clv1-9, AAV CLv-2, AAV CLv-3, AAV CLv-4, AAV CLv-6, AAV CLv-8, AAV CLv-D1, AAV CLv-D2, AAV CLv-D3, AAV CLv-D4, AAV CLv-D5, AAV CLv-D6, AAV CLv-D7, AAV CLv-D8, AAV CLv-E1, AAV CLv-K1, AAV CLv-K3, AAV CLv-K6, AAV CLv-L4, AAV CLv-L5, AAV CLv-L6, AAV CLv-M1, AAV CLv-M11, AAV CLv-M2, AAV CLv-M5, AAV CLv-M6, AAV CLv-M7, AAV CLv-M8, AAV CLv-M9, AAV CLv-R1, AAV CLv-R2, AAV CLv-R3, AAV CLv-R4, AAV CLv-R5, AAV CLv-R6, AAV CLv-R7, AAV CLv-R8, AAV CLv-R9, AAV CSp-1, AAV CSp-10, AAV CSp-11, AAV CSp-2, AAV CSp-3, AAV CSp-4, AAV CSp-6, AAV CSp-7, AAV CSp-8, AAV CSp-8.10, AAV CSp-8.2, AAV CSp-8.4, AAV CSp-8.5, AAV CSp-8.6, AAV CSp-8.7, AAV CSp-8.8, AAV CSp-8.9, AAV CSp-9, AAV.hu.48R3, AAV.VR-355, AAV3B, AAV4, AAV5, AAVF1/HSC1, AAVF11/HSC11, AAVF12/HSC12, AAVF13/HSC13, AAVF14/HSC14, AAVF15/HSC15, AAVF16/HSC16, AAVF17/HSC17, AAVF2/HSC2, AAVF3/HSC3, AAVF4/HSC4, AAVF5/HSC5, AAVF6/HSC6, AAVF7/HSC7, AAVF8/HSC8, and/or AAVF9/HSC9, 7m8, Spark100, AAVMYO and variants thereof.

In some embodiments, the reference AAV capsid sequence comprises an AAV2 sequence. In some embodiments, the reference AAV capsid sequence comprises an AAV5 sequence. In some embodiments, the reference AAV capsid sequence comprises an AAV8 sequence. In some embodiments, the reference AAV capsid sequence comprises an AAV9 sequence. In some embodiments, the reference AAV capsid sequence comprises an AAVrh74 sequence. While not wishing to be bound by theory, it is understood that a reference AAV capsid sequence comprises a VP1 region. In certain embodiments, a reference AAV capsid sequence comprises a VP1, VP2 and/or VP3 region, or any combination thereof. A reference VP1 sequence may be considered synonymous with a reference AAV capsid sequence.

An exemplary reference sequence of wild-type AAV2, SEQ ID NO:1 (wild-type AAV2) is as follows:

(SEQ ID NO: 1)
MAADGYLPDWLEDTLSEGIRQWWKLKPGPPPPKPAERHKDDSRGLVL
PGYKYLGPFNGLDKGEPVNEADAAALEHDKAYDRQLDSGDNPYLKYN
HADAEFQERLKEDTSFGGNLGRAVFQAKKRVLEPLGLVEEPVKTAPG
KKRPVEHSPVEPDSSSGTGKAGQQPARKRLNFGQTGDADSVPDPQPL
GQPPAAPSGLGTNTMATGSGAPMADNNEGADGVGNSSGNWHCDSTWM
GDRVITTSTRTWALPTYNNHLYKQISSQSGASNDNHYFGYSTPWGYF
DFNRFHCHFSPRDWQRLINNNWGFRPKRLNFKLFNIQVKEVTQNDGT
TTIANNLTSTVQVFTDSEYQLPYVLGSAHQGCLPPFPADVFMVPQYG
YLTLNNGSQAVGRSSFYCLEYFPSQMLRTGNNFTFSYTFEDVPFHSS
YAHSQSLDRLMNPLIDQYLYYLSRTNTPSGTTTQSRLQFSQAGASDI
RDQSRNWLPGPCYRQQRVSKTSADNNNSEYSWTGATKYHLNGRDSLV
NPGPAMASHKDDEEKFFPQSGVLIFGKQGSEKTNVDIEKVMITDEEE
IRTTNPVATEQYGSVSTNLQRGNRQAATADVNTQGVLPGMVWQDRDV
YLQGPIWAKIPHTDGHFHPSPLMGGFGLKHPPPQILIKNTPVPANPS
TTFSAAKFASFITQYSTGQVSVEIEWELQKENSKRWNPEIQYTSNYN
KSVNVDFTVDTNGVYSEPRPIGTRYLTRNL

In the sequence above, the sequence found in VP1, VP2 and VP3 is underlined (e.g., a VP3 capsid polypeptide includes, e.g., consists of, amino acids corresponding to amino acids 203-735 of SEQ ID NO:1), the sequence found in both VP1 and VP2 is in bold (e.g., a VP2 capsid polypeptide includes, e.g., consists of, the sequence corresponding to amino acids 138-735 of SEQ ID NO:1) and the sequence that is not underlined or bold is found only in VP1 (e.g., a VP1 capsid polypeptide includes, e.g., consists of, amino acids corresponding to amino acids 1-735 of SEQ ID NO:1).

An example nucleic acid sequence encoding SEQ ID NO:1 is SEQ ID NO:2.

An exemplary reference sequence of wild-type AAV5, SEQ ID NO:3 (wild-type AAV5), is as follows:

(SEQ ID NO: 3)
MSFVDHPPDWLEEVGEGLREFLGLEAGPPKPKPNQQHQDQARGLVLP
GYNYLGPGNGLDRGEPVNRADEVAREHDISYNEQLEAGDNPYLKYNH
ADAEFQEKLADDTSFGGNLGKAVFQAKKRVLEPFGLVEEGAKTAPTG
KRIDDHFPKRKKARTEEDSKPSTSSDAEAGPSGSQQLQIPAQPASSL
GADTMSAGGGGPLGDNNQGADGVGNASGDWHCDSTWMGDRVVTKSTR
TWVLPSYNNHQYREIKSGSVDGSNANAYFGYSTPWGYFDFNRFHSHW
SPRDWQRLINNYWGFRPRSLRVKIFNIQVKEVTVQDSTTTIANNLTS
TVQVFTDDDYQLPYVVGNGTEGCLPAFPPQVFTLPQYGYATLNRDNT
ENPTERSSFFCLEYFPSKMLRTGNNFEFTYNFEEVPFHSSFAPSQNL
FKLANPLVDQYLYRFVSTNNTGGVQFNKNLAGRYANTYKNWFPGPMG
RTQGWNLGSGVNRASVSAFATTNRMELEGASYQVPPQPNGMTNNLQG
SNTYALENTMIFNSQPANPGTTATYLEGNMLITSESETQPVNRVAYN
VGGQMATNNQSSTTAPATGTYNLQEIVPGSVWMERDVYLQGPIWAKI
PETGAHFHPSPAMGGFGLKHPPPMMLIKNTPVPGNITSFSDVPVSSF
ITQYSTGQVTVEMEWELKKENSKRWNPEIQYTNNYNDPQFVDFAPDS
TGEYRTTRPIGTRYLTRPL

Unless otherwise noted, SEQ ID NO:3 is the reference sequence. In the sequence above, the sequence found in VP1, VP2 and VP3 is underlined (e.g., a VP3 capsid polypeptide includes, e.g., consists of, amino acids corresponding to amino acids 193-724 of SEQ ID NO:3), the sequence found in both VP1 and VP2 is in bold (e.g., a VP2 capsid polypeptide includes, e.g., consists of, the sequence corresponding to amino acids 137-724 of SEQ ID NO:3) and the sequence that is not underlined or bold is found only in VP1 (e.g., a VP1 capsid polypeptide includes, e.g., consists of, amino acids corresponding to amino acids 1-724 of SEQ ID NO:3).

The wild-type reference sequence of SEQ ID NO:3 can be encoded by a reference nucleic acid molecule sequence of SEQ ID NO:4.

An exemplary reference sequence of wild-type AAV8, SEQ ID NO:5 (wild-type AAV8), is as follows:

(SEQ ID NO: 5)
MAADGYLPDWLEDNLSEGIREWWALKPGAPKPKANQQKQDDGRGLVL
PGYKYLGPFNGLDKGEPVNAADAAALEHDKAYDQQLQAGDNPYLRYN
HADAEFQERLQEDTSFGGNLGRAVFQAKKRVLEPLGLVEEGAKTAPG
KKRPVEPSPQRSPDSSTGIGKKGQQPARKRLNFGQTGDSESVPDPQP
LGEPPAAPSGVGPNTMAAGGGAPMADNNEGADGVGSSSGNWHCDSTW
LGDRVITTSTRTWALPTYNNHLYKQISNGTSGGATNDNTYFGYSTPW
GYFDFNRFHCHFSPRDWQRLINNNWGFRPKRLSFKLFNIQVKEVTQN
EGTKTIANNLTSTIQVFTDSEYQLPYVLGSAHQGCLPPFPADVFMIP
QYGYLTLNNGSQAVGRSSFYCLEYFPSQMLRTGNNFQFTYTFEDVPF
HSSYAHSQSLDRLMNPLIDQYLYYLSRTQTTGGTANTQTLGFSQGGP
NTMANQAKNWLPGPCYRQQRVSTTTGQNNNSNFAWTAGTKYHLNGRN
SLANPGIAMATHKDDEERFFPSNGILIFGKQNAARDNADYSDVMLTS
EEEIKTTNPVATEEYGIVADNLQQQNTAPQIGTVNSQGALPGMVWQN
RDVYLQGPIWAKIPHTDGNFHPSPLMGGFGLKHPPPQILIKNTPVPA
DPPTTFNQSKLNSFITQYSTGQVSVEIEWELQKENSKRWNPEIQYTS
NYYKSTSVDFAVNTEGVYSEPRPIGTRYLTRNL

In the sequence above, the sequence found in VP1, VP2 and VP3 is underlined (e.g., a VP3 capsid polypeptide includes, e.g., consists of, amino acids corresponding to amino acids 204-738 of SEQ ID NO:5), the sequence found in both VP1 and VP2 is in bold (e.g., a VP2 capsid polypeptide includes, e.g., consists of, the sequence corresponding to amino acids 138-738 of SEQ ID NO:5) and the sequence that is not underlined or bold is found only in VP1 (e.g., a VP1 capsid polypeptide includes, e.g., consists of, amino acids corresponding to amino acids 1-738 of SEQ ID NO:5).

An example nucleic acid sequence encoding SEQ ID NO:5 is SEQ ID NO:6.

An exemplary reference sequence of wild-type AAV9, SEQ ID NO:7 (wild-type AAV9) is as follows:

(SEQ ID NO: 7)
MAADGYLPDWLEDNLSEGIREWWALKPGAPQPKANQQHQDNARGLVL
PGYKYLGPGNGLDKGEPVNAADAAALEHDKAYDQQLKAGDNPYLKYN
HADAEFQERLKEDTSFGGNLGRAVFQAKKRLLEPLGLVEEAAKTAPG
KKRPVEQSPQEPDSSAGIGKSGAQPAKKRLNFGQTGDTESVPDPQPI
GEPPAAPSGVGSLTMASGGGAPVADNNEGADGVGSSSGNWHCDSQWL
GDRVITTSTRTWALPTYNNHLYKQISNSTSGGSSNDNAYFGYSTPWG
YFDFNRFHCHFSPRDWQRLINNNWGFRPKRLNFKLFNIQVKEVTDNN
GVKTIANNLTSTVQVFTDSDYQLPYVLGSAHEGCLPPFPADVFMIPQ
YGYLTLNDGSQAVGRSSFYCLEYFPSQMLRTGNNFQFSYEFENVPFH
SSYAHSQSLDRLMNPLIDQYLYYLSKTINGSGQNQQTLKFSVAGPSN
MAVQGRNYIPGPSYRQQRVSTTVTQNNNSEFAWPGASSWALNGRNSL
MNPGPAMASHKEGEDRFFPLSGSLIFGKQGTGRDNVDADKVMITNEE
EIKTTNPVATESYGQVATNHQSAQAQAQTGWVQNQGILPGMVWQDRD
VYLQGPIWAKIPHTDGNFHPSPLMGGFGMKHPPPQILIKNTPVPADP
PTAFNKDKLNSFITQYSTGQVSVEIEWELQKENSKRWNPEIQYTSNY
YKSNNVEFAVNTEGVYSEPRPIGTRYLTRNL

In the sequence above, the sequence found in VP1, VP2 and VP3 is underlined (e.g., a VP3 capsid polypeptide includes, e.g., consists of, amino acids corresponding to amino acids 203-736 of SEQ ID NO:7), the sequence found in both VP1 and VP2 is in bold (e.g., a VP2 capsid polypeptide includes, e.g., consists of, the sequence corresponding to amino acids 138-736 of SEQ ID NO:7) and the sequence that is not underlined or bold is found only in VP1 (e.g., a VP1 capsid polypeptide includes, e.g., consists of, amino acids corresponding to amino acids 1-736 of SEQ ID NO:7).

An example nucleic acid sequence encoding SEQ ID NO:7 is SEQ ID NO:8.

An exemplary reference sequence of wild-type AAVrh74, SEQ ID NO:9 (wild-type AAVrh74), is as follows:

(SEQ ID NO: 9)
MAADGYLPDWLEDNLSEGIREWWDLKPGAPKPKANQQKQDNGRGLVL
PGYKYLGPFNGLDKGEPVNAADAAALEHDKAYDQQLQAGDNPYLRYN
HADAEFQERLQEDTSFGGNLGRAVFQAKKRVLEPLGLVESPVKTAPG
KKRPVEPSPQRSPDSSTGIGKKGQQPAKKRLNFGQTGDSESVPDPQP
IGEPPAGPSGLGSGTMAAGGGAPMADNNEGADGVGSSSGNWHCDSTW
LGDRVITTSTRTWALPTYNNHLYKQISNGTSGGSTNDNTYFGYSTPW
GYFDFNRFHCHFSPRDWQRLINNNWGFRPKRLNFKLFNIQVKEVTQN
EGTKTIANNLTSTIQVFTDSEYQLPYVLGSAHQGCLPPFPADVFMIP
QYGYLTLNNGSQAVGRSSFYCLEYFPSQMLRTGNNFEFSYNFEDVPF
HSSYAHSQSLDRLMNPLIDQYLYYLSRTQSTGGTAGTQQLLFSQAGP
NNMSAQAKNWLPGPCYRQQRVSTTLSQNNNSNFAWTGATKYHLNGRD
SLVNPGVAMATHKDDEERFFPSSGVLMFGKQGAGKDNVDYSSVMLTS
EEEIKTTNPVATEQYGVVADNLQQQNAAPIVGAVNSQGALPGMVWQN
RDVYLQGPIWAKIPHTDGNFHPSPLMGGFGLKHPPPQILIKNTPVPA
DPPTTFNQAKLASFITQYSTGQVSVEIEWELQKENSKRWNPEIQYTS
NYYKSTNVDFAVNTEGTYSEPRPIGTRYLTRNL

An alternative exemplary reference sequence of SEQ ID NO:11 (alternate wild-type AAVrh74) is as follows:

(SEQ ID NO: 11)
MAADGYLPDWLEDNLSEGIREWWDLKPGAPKPKANQQKQDNGRGLVL
PGYKYLGPFNGLDKGEPVNAADAAALEHDKAYDQQLQAGDNPYLRYN
HADAEFQERLQEDTSFGGNLGRAVFQAKKRVLEPLGLVESPVKTAPG
KKRPVEPSPQRSPDSSTGIGKKGQQPAKKRLNFGQTGDSESVPDPQP
IGEPPAGPSGLGSGTMAAGGGAPMADNNEGADGVGSSSGNWHCDSTW
LGDRVITTSTRTWALPTYNNHLYKQISNGTSGGSTNDNTYFGYSTPW
GYFDFNRFHCHFSPRDWQRLINNNWGFRPKRLNFKLFNIQVKEVTQN
EGTKTIANNLTSTIQVFTDSEYQLPYVLGSAHQGCLPPFPADVFMIP
QYGYLTLNNGSQAVGRSSFYCLEYFPSQMLRTGNNFEFSYNFEDVPF
HSSYAHSQSLDRLMNPLIDQYLYYLSRTQSTGGTAGTQQLLFSQAGP
NNMSAQAKNWLPGPCYRQQRVSTTLSQNNNSNFAWTGATKYHLNGRD
SLVNPGVAMATHKDDEERFFPSSGVLMFGKQGAGKDNVDYSSVMLTS
EEEIKTTNPVATEQYGVVADNLQQQNAAPIVGAVNSQGALPGMVWQN
RDVYLQGPIWAKIPHTDGNFHPSPLMGGFGLKHPPPQILIKNTPVPA
DPPTTFTKAKLASFITQYSTGQVSVEIEWELQKENSKRWNPEIQYTS
NYYKSTNVDFAVNTEGTYSEPRPIGTRYLTRNL

In the sequences above (SEQ ID NO:9 or SEQ ID NO:11), the sequence found in VP1, VP2 and VP3 is underlined (e.g., a VP3 capsid polypeptide includes, e.g., consists of, amino acids corresponding to amino acids 204-738 of SEQ ID NO:9), the sequence found in both VP1 and VP2 is in bold (e.g., a VP2 capsid polypeptide includes, e.g., consists of, the sequence corresponding to amino acids 137-738 of SEQ ID NO:9) and the sequence that is not underlined or bold is found only in VP1 (e.g., a VP1 capsid polypeptide includes, e.g., consists of, amino acids corresponding to amino acids 1-738 of SEQ ID NO:9).

An example nucleic acid sequence encoding SEQ ID NO:9 is SEQ ID NO:10.

The present disclosure refers to structural capsid proteins (including VP1, VP2 and VP3) which are encoded by capsid (Cap) genes. These capsid proteins form an outer protein structural shell (i.e. capsid) of a viral vector such as AAV VP capsid proteins synthesized from Cap polynucleotides generally include a methionine as the first amino acid in the peptide sequence (Met1), which is associated with the start codon (AUG or ATG) in the corresponding Cap nucleotide sequence. However, it is common for a first-methionine (Met1) residue or generally any first amino acid (AA1) to be cleaved off after or during polypeptide synthesis by protein processing enzymes such as Met-aminopeptidases. This “Met/AA− clipping” process often correlates with a corresponding acetylation of the second amino acid in the polypeptide sequence (e.g., alanine, valine, serine, threonine, etc.). Met-clipping commonly occurs with VP1 and VP3 capsid proteins but can also occur with VP2 capsid proteins. Where the Met/AA− clipping is incomplete, a mixture of one or more (one, two or three) VP capsid proteins comprising the viral capsid can be produced, some of which include a Met1/AA1 amino acid (Met+/AA+) and some of which lack a Met1/AA1 amino acid as a result of Met/AA−clipping (Met−/AA−). For further discussion regarding Met/AA−clipping in capsid proteins, see Jin, et al. Direct Liquid Chromatography/Mass Spectrometry Analysis for Complete Characterization of Recombinant Adeno-Associated Virus Capsid Proteins. Hum Gene Ther Methods.2017 Oct.28(5):255-267; Hwang, et al. N-Terminal Acetylation of Cellular Proteins Creates Specific Degradation Signals. Science. 2010 February 19.327(5968): 973-977; the contents of which are each incorporated herein by reference in their entirety. According to the present disclosure, references to capsid polypeptides is not limited to either clipped (Met−/AA−) or unclipped (Met+/AA+) and, in context, also refer to independent capsid polypeptides, viral capsids comprised of a mixture of capsid proteins, and/or polynucleotide sequences (or fragments thereof) which encode, describe, produce or result in capsid polypeptides of the present disclosure. A direct reference to a “capsid polypeptide” (such as VP1, VP2 or VP3) also comprises VP capsid proteins which include a Met1/AA1 amino acid (Met+/AA+) as well as corresponding VP capsid polypeptide which lack the Met1/AA1 amino acid as a result of Met/AA−clipping (Met−/AA−). Further according to the present disclosure, a reference to a specific SEQ ID NO: which comprises one or more capsid polypeptides which include a Met1/AA1 amino acid (Met+/AA+) should be understood to teach the VP capsid polypeptides which lack the Met1/AA1 amino acid as upon review of the sequence, it is readily apparent any sequence which merely lacks the first listed amino acid (whether or not Met1/AA1). As a non-limiting example, reference to a VP1 polypeptide sequence which is 736 amino acids in length and which includes a “Met1” amino acid (Met+) encoded by the AUG/ATG start codon is also understood to teach a VP1 polypeptide sequence which is 735 amino acids in length and which does not include the “Met1” amino acid (Met−) of the 736 amino acid Met+sequence. As a second non-limiting example, reference to a VP1 polypeptide sequence which is 736 amino acids in length and which includes an “AA1” amino acid (AA1+) encoded by any NNN initiator codon can also be understood to teach a VP1 polypeptide sequence which is 735 amino acids in length and which does not include the “AA1” amino acid (AA1−) of the 736 amino acid AA1+sequence. References to viral capsids formed from VP capsid proteins (such as reference to specific AAV capsid serotypes), can incorporate VP capsid proteins which include a Met1/AA1 amino acid (Met+/AA1+), corresponding VP capsid proteins which lack the Met1/AA1 amino acid as a result of Met/AA1-clipping (Met−/AA1−), and combinations thereof (Met+/AA1+ and Met−/AA1−). As a non-limiting example, an AAV capsid serotype can include VP1 (Met+/AA1+), VP1 (Met−/AA1−), or a combination of VP1 (Met+/AA1+) and VP1 (Met−/AA1−). An AAV capsid serotype can also include VP3 (Met+/AA1+), VP3 (Met−/AA1−), or a combination of VP3 (Met+/AA1+) and VP3 (Met−/AA1−); and can also include similar optional combinations of VP2 (Met+/AA1) and VP2 (Met−/AA1−).

In some embodiments, the reference AAV capsid sequence comprises an amino acid sequence with 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identity to any of the those described above.

In some embodiments, a capsid polypeptide of the disclosure is an isolated or purified polypeptide (e.g., isolated or purified from a cell, other biological component, or contaminant). In some embodiments, the variant polypeptide is present in a dependoparvovirus particle, e.g., described herein.

In some embodiments, the variant capsid polypeptide is present in a cell, cell-free system, or translation system, e.g., described herein.

In some embodiments, the capsid polypeptide is present in a dependoparvovirus B (e.g., AAV9) particle. In some embodiments, the capsid particle has increased CNS transduction as compared to a reference capsid polypeptide, e.g., a wild-type capsid polypeptide such as a VP1 capsid polypeptide of SEQ ID NO:7 or a VP2 or VP3 portion thereof. In some embodiments, the capsid particle has increased muscle transduction as compared to a reference capsid polypeptide, e.g., a wild-type capsid polypeptide such as a VP1 capsid polypeptide of SEQ ID NO:7 or a VP2 or VP3 portion thereof.

### 6.2.1. Targeting Peptides

The capsid polypeptides of the disclosure can include (but do not necessarily include) a targeting peptide to alter the tropism of the capsid polypeptides, for example to enhance targeting to the CNS or muscle tissue. Thus, in some embodiments, a capsid polypeptide of the disclosure includes a targeting peptide. In other embodiments, a capsid polypeptide of the disclosure does not include a targeting peptide.

### 6.2.1.1. CNS Targeting Peptides

Various targeting peptides for enhancing CNS tropism, and which can be included in capsid polypeptides of the disclosure, are described in the art, for example in WO 2017/197355, WO 2019/006182, WO 2019/060454, WO 2012/145601, WO 2018/022905, WO 2021/243085, WO 2019/076856, WO 2015/038958, WO 2015/191508, WO 2020/068990, WO 2020/210655, WO 2020/198737, WO 2020/028751, WO 2019/028306, WO 2017/100671 A1, WO 2020/028751 A2, WO 2020/072683 A1, WO 2020/160337 A1, WO 2020/223280 A1, WO 2021/025995 A1, WO 2021/202651 A1, WO 2021/230987 A1, WO 2022/235702 A1, WO 2020/014471, WO 2018/189244, WO 2019/141765, WO 2019/207132, WO 2019/210267, WO 2018/156654, WO 2010/093784, WO 2015/048534, WO 2017/058892, WO 2019/169132, WO 2021/108468, WO 2021/102234, WO 2022/173847, WO 2021/077000, WO 2020/160337, WO 2021/050974, WO 2021/222831, WO 2022/020616, WO 2020/193799, WO 2021/072197, WO 2022/126188, WO 2022/126189, WO 2021/165544, WO 2021/084133, WO 2022/040527, WO 2022/221400, WO 2022/221404, WO 2022/221420, WO 2021/216456, WO 2021/009684, WO 2021/242909, WO 2019/158619, WO 2021/226267, WO 2023/283962, WO 2021/219762, WO 2022/226374, WO 2022/226375, WO 2022/229703, and WO 2022/229702, the contents of which are incorporated herein by reference in their entireties. Targeting peptides are typically 3 to 20 amino acids in length. In some embodiments, a targeting peptide is 3 to 12 amino acids in length. In other embodiments, a targeting peptide is 5 to 12 amino acids in length. In other embodiments, a targeting peptide is 5 to 10 amino acids in length. In other embodiments, a targeting peptide is 7 to 10 amino acids in length. In some embodiments, a targeting peptide is 7 amino acids in length. In other embodiments, a targeting peptide is 9 amino acids in length.

In some embodiments, the targeting peptide comprises at least 3, 4, 5, 6, 7, 8, or 9 consecutive amino acids from the amino acid sequence of PLNGAVHLY (SEQ ID NO: 103). In some embodiments, the targeting peptide comprises the amino acid sequence PLNGAVHLY (SEQ ID NO: 103). In some embodiments, the targeting peptide comprises at least 3, 4, 5, 6, or 7 consecutive amino acids from the amino acid sequence of IVMNSLK (SEQ ID NO: 104). In some embodiments, the targeting peptide comprises the amino acid sequence IVMNSLK (SEQ ID NO: 104). In some embodiments, the targeting peptide comprises at least 3, 4, 5, 6, or 7 consecutive amino acids from the amino acid sequence of RDSPKGW (SEQ ID NO: 105). In some embodiments, the targeting peptide comprises the amino acid sequence RDSPKGW (SEQ ID NO: 105). In some embodiments, the targeting peptide comprises at least 3, 4, 5, 6, or 7 consecutive amino acids from the amino acid sequence of YSTDVRM (SEQ ID NO: 106). In some embodiments, the targeting peptide comprises the amino acid sequence YSTDVRM (SEQ ID NO: 106). In some embodiments, the targeting peptide comprises at least 3, 4, 5, 6, or 7 consecutive amino acids from the amino acid sequence of RESPRGL (SEQ ID NO: 107). In some embodiments, the targeting peptide comprises the amino acid sequence RESPRGL (SEQ ID NO: 107). In some embodiments, the targeting peptide comprises 4, 5, 6, or 7 consecutive amino acids from GNNTRSV (SEQ ID NO: 108), GNNTRDT (SEQ ID NO: 109) or TNSTRPV (SEQ ID NO: 110). In some embodiments, the targeting peptide comprises the amino acid sequence GNNTRSV (SEQ ID NO: 108). In some embodiments, the targeting peptide comprises the amino acid sequence GNNTRDT (SEQ ID NO: 109). In some embodiments, the targeting peptide comprises the amino acid sequence TNSTRPV (SEQ ID NO: 110).

In some embodiments, the CNS-targeting peptide is present in, e.g., inserted into, loop VIII of the capsid polypeptide. In some embodiments, the targeting peptide is inserted at any amino acid position corresponding to positions 586-592, inclusive, of the wild-type capsid polypeptide (e.g., SEQ ID NO:7). For example, the targeting peptide can be inserted between amino acids 588-589 (positions corresponding to the wild-type capsid polypeptide (e.g., SEQ ID NO:7)). In some embodiments, the targeting peptide is present, e.g., inserted, immediately subsequent to the position corresponding to 586, 588, or 589 of the wild-type capsid polypeptide (e.g., SEQ ID NO:7). In some embodiments, the capsid polypeptide further comprises a deletion at the position corresponding to 587 and/or a deletion at the position corresponding to 588 of the wild-type capsid polypeptide (e.g., SEQ ID NO:7).

### 6.2.1.2. Muscle Targeting Peptides

Various targeting peptides for enhancing skeletal muscle tropism, and which can be included in capsid polypeptides of the disclosure, are described in the art, for example in WO 2020/206189A1, WO 2022/226374 A1, WO 2022/053630 A1, and WO 2019/207132 A1.

In some embodiments, the muscle targeting peptide comprises at least 3, 4, 5, 6, or 7 consecutive amino acids from the amino acid sequence ASSLNIA (SEQ ID NO: 208).

In some aspects, the targeting peptide targets the insulin receptor (INSR). In some embodiments, the peptide targeting the INSR comprises an amino acid sequence having at least 80%, 87%, 91%, 94%, 97%, or 100% sequence identity to SLEEEWAQVECEVYGRGCPSGSLDESFYDWFERQL (SEQ ID NO: 111), or which has at least 25, 26, 27, 28, 29, 30, 31, 32, 33, 34 or 35 consecutive amino acids from the amino acid sequence

(SEQ ID NO: 111)
SLEEEWAQVECEVYGRGCPSGSLDESFYDWFERQL.

In some aspects, the targeting peptide targets muscle-specific kinase (MUSK). In some embodiments, the inserted MUSK-targeting peptide is from the acetylcholinesterase collagenic tail peptide (CoIQ), e.g., a C-terminal portion of CoIQ (CoIQ CTD). In some embodiments, the CoIQ CTD peptide comprises an amino acid sequence having at least 80%, 85%, 90%, 95%, 96%, 97%, 98%, 99%, or 100% sequence identity to TPFYPVGYTVKQPGTCGDGVLQPGEECDDGNPDVSDGCIDCHRAYCGDGYRHQGVEDCDGSDFGYLT CETYLPGSYGDLRCTQYCSIDSTPCRYFT (SEQ ID NO: 112), or which has at least 70, 80, 85, 90, 91, 92, 93, 94, 95, or 96 consecutive amino acids from the amino acid sequence

(SEQ ID NO: 112)
TPFYPVGYTVKQPGTCGDGVLQPGEECDDGNPDVSDGCIDCHRAYCG
DGYRHQGVEDCDGSDFGYLTCETYLPGSYGDLRCTQYCSIDSTPCRY
FT.

In some aspects, the targeting peptide targets integrin, for example via an RGD-motif. In some embodiments, the RGD peptide comprises a subsequence Y or F amino acid to produce an RGDY (SEQ ID NO: 209) or RGDF motif (SEQ ID NO: 210). In some embodiments, integrin targeting peptides have the motif RGDX1X2X3X4, with X1 to X4 each being any amino acid. In various aspects of the motif RGDX1X2X3X4, X1, X2, and X3 are each independently selected from L, G, V, and A and/or X4 is S, V, A, G, or L. In some embodiments, at least one of X2 and X3 is G. In some embodiments, the targeting peptide comprises the amino acid sequence RGDLGLS (SEQ ID NO: 113). In some embodiments, the targeting peptide comprises the amino acid sequence RGDLSTP (SEQ ID NO: 114). In some embodiments, the targeting peptide comprises the amino acid sequence SNSRGDYNSL (SEQ ID NO: 115). In some embodiments, the targeting peptide comprises the amino acid sequence ENRRGDFNNT (SEQ ID NO: 116). In some embodiments, the targeting peptide comprises the amino acid sequence SRGDYNSL (SEQ ID NO: 117). In some embodiments, the targeting peptide comprises the amino acid sequence RGDYNSL (SEQ ID NO: 118). In some embodiments, the targeting peptide comprises the amino acid sequence RGDLST (SEQ ID NO: 119). In some embodiments, the targeting peptide comprises the amino acid sequence RGDYVGL (SEQ ID NO: 120). In some embodiments, the targeting peptide comprises the amino acid sequence RGDAVGV (SEQ ID NO: 121). The RGD peptides can be inserted in a linker, e.g., a flexible linker such as GGGS (SEQ ID NO: 211), scaffold. Other suitable linker scaffolds can be found in WO 2022/226374 A1, pages 26-28 of which are incorporated herein by reference.

In some embodiments, the skeletal muscle-targeting peptide is present in, e.g., inserted into, loop VIII of the capsid polypeptide. In some embodiments, the targeting peptide is inserted at any amino acid position corresponding to positions 586-592, inclusive, of the wild-type capsid polypeptide (e.g., SEQ ID NO:7). For example, the targeting peptide can be inserted between amino acids 588-589 (positions corresponding to the wild-type capsid polypeptide (e.g., SEQ ID NO:7)). In some embodiments, the targeting peptide is present, e.g., inserted, immediately subsequent to the position corresponding to 586, 588, or 589 of the wild-type capsid polypeptide (e.g., SEQ ID NO:7). In some embodiments, the capsid polypeptide further comprises a deletion at the position corresponding to 587 and/or a deletion at the position corresponding to 588 of the wild-type capsid polypeptide (e.g., SEQ ID NO:7).

### 6.3. Nucleic Acids

The disclosure is further directed, in part, to a nucleic acid comprising a sequence encoding a variant capsid polypeptide as provided for herein, e.g., as described in Section 6.2. In some embodiments the nucleic acid encodes a VP1 variant capsid polypeptide. In some embodiments, the nucleic acid encodes a VP2 variant capsid polypeptide. In some embodiments, the nucleic acid encodes a VP3 variant capsid polypeptide. In some embodiments, the nucleic acid encodes a VP1, VP2 and VP3 variant capsid polypeptide.

Accordingly, in some embodiments, the disclosure provides a nucleic acid comprising a nucleotide sequence encoding a variant capsid polypeptide that comprises one or more of the mutation differences or the entire mutation set associated with the capsid polypeptide of SEQ ID NO:12, 14, 16, or 18.

Typically, the nucleic acid comprises a nucleotide sequence that encodes a variant capsid polypeptide comprising one or more mutation differences or the entire mutation set associated with the capsid polypeptide of SEQ ID NO:12, 14, 16, or 18, and has at least 80%, at least 85%, at least 90%, at least 91%, at least 92%, at least 93%, at least 94%, at least 95%, at least 96%, at least 97%, at least 98%, or at least 99% sequence identity to a reference AAV serotype, e.g., as described herein, e.g., to SEQ ID NO:7.

In various embodiments, the nucleic acid comprises a nucleotide sequence that encodes a variant capsid polypeptide which, but for the mutation differences or mutation set associated with the capsid polypeptide of SEQ ID NO:12, 14, 16, or 18, is at least 90%, at least 95%, 96%, 97%, 98%, 99%, or 100% identical to a reference AAV serotype described herein.

In some embodiments, the nucleic acid comprises a nucleotide sequence that encodes a variant capsid polypeptide which, but for the mutation differences associated with the capsid polypeptide of SEQ ID NO:12, 14, 16, or 18, is at least 90%, at least 95%, 96%, 97%, 98%, 99%, or 100% identical to a capsid polypeptide of SEQ ID NO:1 (e.g., a VP1, VP2 or VP3 sequence of SEQ ID NO:1).

In some embodiments, the nucleic acid comprises a nucleotide sequence that encodes a variant capsid polypeptide which, but for the mutation differences associated with the capsid polypeptide of SEQ ID NO:12, 14, 16, or 18, is at least 90%, at least 95%, 96%, 97%, 98%, 99%, or 100% identical to a capsid polypeptide of SEQ ID NO:3 (e.g., a VP1, VP2 or VP3 sequence of SEQ ID NO:3).

In some embodiments, the nucleic acid comprises a nucleotide sequence that encodes a variant capsid polypeptide which, but for the mutation differences associated with the capsid polypeptide of SEQ ID NO:12, 14, 16, or 18, is at least 90%, at least 95%, 96%, 97%, 98%, 99%, or 100% identical to a capsid polypeptide of SEQ ID NO:5 (e.g., a VP1, VP2 or VP3 sequence of SEQ ID NO:5).

In some embodiments, the nucleic acid comprises a nucleotide sequence that encodes a variant capsid polypeptide which, but for the mutation differences associated with the capsid polypeptide of SEQ ID NO:12, 14, 16, or 18, is at least 90%, at least 95%, 96%, 97%, 98%, 99%, or 100% identical to a capsid polypeptide of SEQ ID NO:7 (e.g., a VP1, VP2 or VP3 sequence of SEQ ID NO:7).

In some embodiments, the nucleic acid comprises a nucleotide sequence that encodes a variant capsid polypeptide which, but for the mutation differences associated with the capsid polypeptide of SEQ ID NO:12, 14, 16, or 18, is at least 90%, at least 95%, 96%, 97%, 98%, 99%, or 100% identical to a capsid polypeptide of SEQ ID NO:9 (e.g., a VP1, VP2 or VP3 sequence of SEQ ID NO:9).

In some embodiments, the nucleic acid comprises a nucleotide sequence that encodes a variant capsid polypeptide which, but for the mutation differences associated with the capsid polypeptide of SEQ ID NO:12, 14, 16, or 18, is at least 90%, at least 95%, 96%, 97%, 98%, 99%, or 100% identical to a capsid polypeptide of SEQ ID NO:11 (e.g., a VP1, VP2 or VP3 sequence of SEQ ID NO:11).

In some embodiments, the nucleic acid comprises a nucleotide sequence that encodes a variant capsid polypeptide which is at least 70%, 75%, 80%, 85%, 90%, 91%, 92%, 93%, 94%, 95%, 96%, 97%, 98%, 99%, or 100% identical to the amino acid sequence of a variant capsid polypeptide as provided herein, e.g., a VP1 capsid polypeptide of the capsid polypeptide of SEQ ID NO:12, 14, 16, or 18 or a VP2 or VP3 portion thereof.

In some embodiments, the nucleic acid comprises a nucleotide sequence that encodes a variant capsid polypeptide that is at least 70%, 75%, 80%, 85%, 90%, 91%, 92%, 93%, 94%, 95%, 96%, 97%, 98%, 99%, or 100% identical to a variant capsid polypeptide as provided herein.

In some embodiments, the nucleic acid comprises a nucleotide sequence that encodes a variant capsid polypeptide comprising a VP1, VP2, VP3, or any combination thereof, that each has about 1 to about 20 mutations as compared to a VP1 polypeptide of the capsid polypeptide of SEQ ID NO:12, 14, 16, or 18, and comprises one or more of the mutation differences or the entire mutation set of the capsid polypeptide of SEQ ID NO:12, 14, 16, or 18.

In some embodiments, the nucleic acid comprises a nucleotide sequence that encodes a variant capsid polypeptide comprising a VP1, VP2, VP3, or any combination thereof, that each has about 1 to about 10 mutations as compared to a VP1 polypeptide of the capsid polypeptide of SEQ ID NO:12, 14, 16, or 18, and comprises one or more of the mutation differences or the entire mutation set of the capsid polypeptide of SEQ ID NO:12, 14, 16, or 18.

In some embodiments, the nucleic acid comprises a nucleotide sequence that encodes a variant capsid polypeptide comprising a VP1, VP2, VP3, or any combination thereof, that each has about 1 to about 5 mutations as compared to a VP1 polypeptide of the capsid polypeptide of SEQ ID NO:12, 14, 16, or 18, and comprises one or more of the mutation differences or the entire mutation set of the capsid polypeptide of SEQ ID NO:12, 14, 16, or 18.

In some embodiments, the nucleic acid comprises a nucleotide sequence of SEQ ID NO:13 (which encodes a capsid polypeptide of SEQ ID NO:12). In some embodiments, the nucleic acid comprises a nucleotide sequence of SEQ ID NO:15 (which encodes a capsid polypeptide of SEQ ID NO:14). In some embodiments, the nucleic acid comprises a nucleotide sequence of SEQ ID NO:17 (which encodes a capsid polypeptide of SEQ ID NO:16). In some embodiments, the nucleic acid comprises a nucleotide sequence of SEQ ID NO:19 (which encodes a capsid polypeptide of SEQ ID NO:18). In some embodiments, the nucleic acid comprises a nucleotide sequence which encodes a capsid polypeptide of any one of SEQ ID NOS:300-445.

In some embodiments, a nucleic acid of the disclosure (e.g., encoding a variant capsid polypeptide as described in Section 6.2) comprises conventional control elements or sequences which are operably linked to the nucleic acid molecule in a manner which permits transcription, translation and/or expression in a cell transfected with the nucleic acid (e.g., a plasmid vector comprising said nucleic acid) or infected with a virus comprising said nucleic acid. As used herein, “operably linked” sequences include both expression control sequences that are contiguous with the gene of interest and expression control sequences that act in trans or at a distance to control the gene of interest.

### 6.4. Dependoparvovirus Particles

The disclosure is also directed, in part, to a dependoparvovirus particle (e.g., a functional dependoparvovirus particle) comprising a nucleic acid or polypeptide described herein or produced by a method described herein.

In some embodiments, the viral particle comprising a variant capsid polypeptide, e.g., a variant capsid polypeptide described herein, exhibits increased muscle (cardiac and/or skeletal) transduction as compared to a viral particle with the wild-type capsid polypeptide (SEQ ID NO:7).

In some embodiments, a dependoparvovirus particle comprises an amino acid sequence that has at least 80, 85, 90, 91, 92, 93, 94, 95, 96, 97, 98, 99, or 100% identity to the amino acid sequences provided for herein (e.g., a capsid polypeptide described in Section 6.2 such as the VP1 capsid polypeptide of SEQ ID NO:12, 14, 16, or 18 or a VP2 or VP3 portion thereof). In some embodiments, the variant capsid polypeptide comprises an amino acid sequence that differs by no more than 30, 29, 28, 27, 26, 25, 24, 23, 22, 21, 20, 19, 18, 17, 16, 15, 14, 13, 12, 11, 10, 9, 8, 7, 6, 5, 4, 3, 2, or 1 amino acids from the amino acid sequence of a variant capsid polypeptide provided for herein.

In some embodiments, the additional alteration improves a production characteristic of a dependoparvovirus particle or method of making the same. In some embodiments, the additional alteration improves or alters another characteristic of a dependoparvovirus particle, e.g., tropism.

Dependoparvovirus is a single-stranded DNA parvovirus that grows only in cells in which certain functions are provided, e.g., by a co-infecting helper virus. Several species of dependoparvovirus are known, including dependoparvovirus A and dependoparvovirus B, which include serotypes known in the art as adeno-associated viruses (AAV). At least thirteen serotypes of AAV that have been characterized. General information and reviews of AAV can be found in, for example, Carter, Handbook of Parvoviruses, Vol. 1, pp. 169-228 (1989), and Berns, Virology, pp. 1743-1764, Raven Press, (New York, 1990). AAV serotypes, and to a degree, dependoparvovirus species, are significantly interrelated structurally and functionally. (See, for example, Blacklowe, pp. 165-174 of Parvoviruses and Human Disease, J. R. Pattison, ed. (1988); and Rose, Comprehensive Virology 3:1-61 (1974)). For example, all AAV serotypes apparently exhibit very similar replication properties mediated by homologous rep genes; and all bear three related capsid proteins. In addition, heteroduplex analysis reveals extensive cross-hybridization between serotypes along the length of the genome, further suggesting interrelatedness. Dependoparvoviruses genomes also comprise self-annealing segments at the termini that correspond to “inverted terminal repeat sequences” (ITRs).

The genomic organization of naturally occurring dependoparvoviruses, e.g., AAV serotypes, is very similar. For example, the genome of AAV is a linear, single-stranded DNA molecule that is approximately 5,000 nucleotides (nt) in length or less. Inverted terminal repeats (ITRs) flank the unique coding nucleotide sequences for the non-structural replication (Rep) proteins and the structural capsid (Cap) proteins. Three different viral particle (VP) proteins form the capsid. The terminal approximately 145 nt of the genome are self-complementary and are organized so that an energetically stable intramolecular duplex forming a T-shaped hairpin may be formed. These hairpin structures function as an origin for viral DNA replication, serving as primers for the cellular DNA polymerase complex. The Rep genes encode the Rep proteins: Rep78, Rep68, Rep52, and Rep40. Rep78 and Rep68 are transcribed from the p5 promoter, and Rep 52 and Rep40 are transcribed from the p19 promoter. The cap genes encode the VP proteins, VP1, VP2, and VP3. The cap genes are transcribed from the p40 promoter.

In some embodiments, a dependoparvovirus particle of the disclosure comprises a nucleic acid comprising a variant capsid polypeptide provided for herein. In some embodiments, the particle comprises a polypeptide as provided for herein.

In some embodiments, the dependoparvovirus particle of the disclosure is an AAV9 particle. In some embodiments, the AAV9 particle comprises a variant capsid polypeptide as provided for herein or a nucleic acid molecule encoding the same.

In some embodiments the dependoparvovirus particle comprises a variant capsid comprising a variant capsid polypeptide described herein. In some embodiments, the dependoparvovirus particle comprises variant capsid polypeptide described herein and a nucleic acid molecule. In some embodiments, the dependoparvovirus particle comprises variant capsid polypeptide described herein and a nucleic acid molecule comprising one or more inverted terminal repeat sequences (ITRs), for example, ITRs derived from an AAV9 dependoparvovirus or an AAV2 dependoparvovirus, one or more regulatory elements (for example, a promoter), and a payload (e.g., as described herein, e.g., a heterologous transgene). In some embodiments, at least one of the ITRs is modified. In some embodiments, the nucleic acid molecule is single-stranded. In some embodiments, the nucleic acid molecule is double stranded, for example, self-complementary. Various ITRs and their use in self-complementary AAV vectors are recognized in the art and include those described in U.S. Pat. Nos. 7,465,583, 8,298,818 and 9,150,882; in U.S. Patent Application Publication Nos. 2004/0029106 A1, 2023/0139985 A1, 2022/0175887 A1; and in Wilmott et al., 2019, Hum. Gene. Ther. Methods, 30(6):206-213 and McCarty, 2008, Mol. Ther., 16(10):1648-1656, each incorporated herein by reference.

### 6.5. Improved Biodistribution and Transduction Characteristics

The disclosure is directed, in part, to nucleic acids, polypeptides, cells, cell free systems, translation systems, viral particles, and methods associated with using and making the same to produce viral particles that have increased distribution to CNS tissues and cells and/or CNS tissue transduction, and/or increased distribution to muscle tissues and cells and/or muscle tissue transduction as compared to a viral particle comprising a reference sequence that does not otherwise comprise the mutations described herein (or mutations corresponding thereto), for example, as compared with a viral particle comprising a capsid polypeptide sequence of SEQ ID NO:7. In some embodiments, a use of a viral particle (e.g., a viral particle as described in Section 6.4) comprising the variant capsid polypeptides as described in Section 6.2 or any one of numbered embodiments 1 to 326 leads to increased CNS biodistribution of the viral particle and/or increased transduction (e.g., brain biodistribution and/or transduction) of a transgene virus particle in the cells of the CNS, and, therefore, increased expression of the payload (transgene) in the CNS. In some embodiments, a use of a viral particle (e.g., a viral particle as described in Section 6.4) comprising the variant capsid polypeptides as described in Section 6.2 or any one of numbered embodiments 1 to 326 leads to increased muscle biodistribution of the viral particle and/or increased transduction (e.g., cardiac or skeletal biodistribution and/or transduction) of a transgene virus particle in the cells of the muscle, and, therefore, increased expression of the payload (transgene) in the muscle.

In some embodiments, use of a viral particle (e.g., a viral particle as described in Section 6.4) comprising the variant capsid polypeptides as described in Section 6.2 or any one of numbered embodiments 1 to 326 further leads to reduced (or non-increased) biodistribution of the viral particle and/or reduced (or non-increased) transduction of the transgene in one or more peripheral tissues, e.g., liver.

In some embodiments, biodistribution and transduction (e.g., of the tissue types described in this section) are measured as described herein, for example as described in Section 8.1 (e.g., Example 1) for example by relative quantification (e.g., via qPCR) of transgene mRNA in one or more samples isolated from the relevant tissue type, e.g., CNS or muscle. In some embodiments, biodistribution and/or transduction of a virus particle having a variant capsid polypeptide can be measured using virus particles having a transgene operably linked to a promoter that is active in a target cell or tissue type of interest. In some embodiments, the promoter is a ubiquitous promoter. In other embodiments, the promoter is selective or specific to a target cell or tissue type (e.g., CNS and/or muscle), and, optionally, is less (or not) active in a cell or tissue type where transgene expression is not desired (e.g., liver). A promoter that is selective for a first cell or tissue type over a second cell or tissue type is active in the first cell or tissue type and less active or silent in the second cell or tissue type. In some embodiments, the promoter is a CNS-specific or CNS-selective promoter. In some embodiments, the promoter is a muscle-specific promoter-specific or muscle-selective promoter. In some embodiments, the promoter is active in both CNS and muscle tissue. In some embodiments, biodistribution and/or transduction of a virus particle having a variant capsid polypeptide can be measured using virus particles having a transgene operably linked to a ubiquitous promoter or a CNS-specific promoter. For example, biodistribution can be measured using virus particles having a transgene operably linked to CBh promoter or a hSYN promoter. In various embodiments, the transgene is a transgene encoding a capsid polypeptide or any other suitable heterologous transgene, for example a nucleic acid sequence encoding a synthetic, mammalian or human therapeutic protein or nucleic acid (e.g., mRNA or RNAi) or reporter gene such as, for example a nucleic acid encoding a GFP or mCherry reporter.

In some embodiments, the virus particle, e.g., as described herein, e.g., comprising a variant capsid polypeptide described herein, exhibits increased transduction of myocytes (muscle cells) relative to a virus particle comprising a reference capsid polypeptide, e.g., a reference capsid polypeptide of SEQ ID NO:7.

In some embodiments, a viral particle comprising the variant capsid polypeptide, e.g., the variant capsid polypeptide described herein, exhibits improved properties, e.g., improved biodistribution, transduction and/or production. Unless indicated otherwise, improvement rates are presented as fold-improvement over the rates exhibited by a virus particle comprising capsid polypeptides of SEQ ID NO:7. In some embodiments, improvement means an increase, e.g., in the case of CNS and/or muscle biodistribution or transduction. In other embodiments, improvement means a decrease, e.g., in the case of liver biodistribution or liver transduction. A virus particle having increased biodistribution or transduction in target cells or target tissue types, e.g., muscle, and/or decreased biodistribution or transduction in off-target cells or off-target tissue types, e.g., liver, may have improved specificity for a target cell or target tissue type. This improvement may be beneficial in the use of the viral particle to deliver a therapeutic transgene to the target cell or target tissue type in a subject afflicted with a disease affecting the target cell or target tissue type, for example.

In some embodiments, one or more improved properties (e.g., increased or decreased biodistribution and/or transduction) is exhibited in a mammal, e.g., a primate, e.g., a human. In some embodiments, the increased or decreased biodistribution and/or transduction is exhibited upon administration of the virus particle or pharmaceutical composition comprising the virus particle, e.g., as described herein, by systemic administration, e.g., intravenous administration.

### 6.6. Methods of Making Compositions Described Herein

The disclosure is directed, in part, to a method of making a dependoparvovirus particle, e.g., a dependoparvovirus particle described herein. In some embodiments, a method of making dependoparvovirus particle comprises providing a cell, cell-free system, or other translation system, comprising a nucleic acid described encoding a variant capsid polypeptide provided for herein, or a polypeptide provided for herein (e.g., a variant capsid polypeptide); and cultivating the cell, cell-free system, or other translation system under conditions suitable for the production of the dependoparvovirus particle, thereby making the dependoparvovirus particle.

In some embodiments, a nucleic acid or polypeptide described herein is produced by a method known to one of skill in the art. In some embodiments, the nucleic acids, polypeptides, and fragments thereof of the disclosure are produced by any suitable means, including recombinant production, chemical synthesis, or other synthetic means. Such production methods are within the knowledge of those of skill in the art and are not a limitation of the present invention.

### 6.6.1. Host Cells

Aspects of the disclosure are directed to a host cell comprising a nucleic acid of the disclosure (e.g., encoding a variant capsid polypeptide as described in Section 6.2). A host cell of the disclosure, e.g., a host cell useful to general AAV virus particles comprising a variant AAV capsid as described herein, generally comprises one or more nucleic acids comprising a coding sequence encoding a variant capsid polypeptide of the disclosure (e.g., as described in Section 6.2), together with a payload (e.g., transgene) and one or more coding sequences encoding additional components useful for promoting packaging of the payload into a dependoparvovirus capsid. Additional components include, for example, coding sequences for a rep protein and dependoparvovirus inverted terminal repeats (ITRs), as well as helper sequences which promote dependoparvovirus particle production and/or secretion. Examples of helper sequences include E1a, E1b, E2a, E4, and VA. Such helper sequences may be included endogenously within the host cell (e.g., the host cell may be engineered to express such helper sequences, e.g., integrated into the host cell genome) or may be provided exogenously (e.g., transduced on the same or a different nucleic acid as the variant capsid polypeptide, payload, rep, and/or ITRs).

In some embodiments, the helper sequences include AdV5 helper sequences. An exemplary AdV5 genome is disclosed in the art as NCBI Reference Sequence AC_000008.1 (disclosed as SEQ ID NO:1 of PCT Patent Application Publication No. WO/2022/079429 A1). In some embodiments, a host cell disclosed herein comprises a portion of an AdV5 genome encoding for one or more helper protein sequences (e.g., E1a, E1b, E2a, E4) or RNA sequences (e.g., VA). In some embodiments, a host cell disclosed herein comprises a nucleic acid sequence encoding one or more AdV5 helper protein sequences. Certain exemplary AdV5 helper protein sequences are provided below.

AdV5 E1A:
(SEQ ID NO: 212)
MRHIICHGGVITEEMAASLLDQLIEEVLADNLPPPSHFEPPTLHELY
DLDVTAPEDPNEEAVSQIFPDSVMLAVQEGIDLLTFPPAPGSPEPPH
LSRQPEQPEQRALGPVSMPNLVPEVIDLTCHEAGFPPSDDEDEEGEE
FVLDYVEHPGHGCRSCHYHRRNTGDPDIMCSLCYMRTCGMFVYSPVS
EPEPEPEPEPEPARPTRRPKMAPAILRRPTSPVSRECNSSTDSCDSG
PSNTPPEIHPVVPLCPIKPVAVRVGGRRQAVECIEDLLNEPGQPLDL
SCKRPRP
AdV5 E1B 19K:
(SEQ ID NO: 213)
MEAWECLEDFSAVRNLLEQSSNSTSWFWRFLWGSSQAKLVCRIKEDY
KWEFEELLKSCGELFDSLNLGHQALFQEKVIKTLDFSTPGRAAAAVA
FLSFIKDKWSEETHLSGGYLLDFLAMHLWRAVVRHKNRLLLLSSVRP
AIIPTEEQQQQQEEARRRRQEQSPWNPRAGLDPRE
AdV5 E1B 55K:
(SEQ ID NO: 214)
MERRNPSERGVPAGFSGHASVESGCETQESPATVVFRPPGDNTDGGA
AAAAGGSQAAAAGAEPMEPESRPGPSGMNVVQVAELYPELRRILTIT
EDGQGLKGVKRERGACEATEEARNLAFSLMTRHRPECITFQQIKDNC
ANELDLLAQKYSIEQLTTYWLQPGDDFEEAIRVYAKVALRPDCKYKI
SKLVNIRNCCYISGNGAEVEIDTEDRVAFRCSMINMWPGVLGMDGVV
IMNVRFTGPNFSGTVFLANTNLILHGVSFYGFNNTCVEAWTDVRVRG
CAFYCCWKGVVCRPKSRASIKKCLFERCTLGILSEGNSRVRHNVASD
CGCFMLVKSVAVIKHNMVCGNCEDRASQMLTCSDGNCHLLKTIHVAS
HSRKAWPVFEHNILTRCSLHLGNRRGVFLPYQCNLSHTKILLEPESM
SKVNLNGVFDMTMKIWKVLRYDETRTRCRPCECGGKHIRNQPVMLDV
TEELRPDHLVLACTRAEFGSSDEDTD
AdV5 E3 12.5K:
(SEQ ID NO: 215)
MLSGEAEQLRLKHLVHCRRHKCFARDSGEFCYFELPEDHIEGPAHGV
RLTAQGELARSLIREFTQRPLLVERDRGPCVLTVICNCPNLGLHQDL
CCHLCAEYNKYRN
AdV5 E3 CR1-alpha0:
(SEQ ID NO: 216)
MNNSSNSTGYSNSGFSRIGVGVILCLVILFILILTLLCLRLAACCVH
ICIYCQLFKRWGRHPR
AdV5 E3 gp19K:
(SEQ ID NO: 217)
MIRYIILGLLTLASAHGTTQKVDFKEPACNVTFAAEANECTTLIKCT
TEHEKLLIRHKNKIGKYAVYAIWQPGDTTEYNVTVFQGKSHKTFMYT
FPFYEMCDITMYMSKQYKLWPPQNCVENTGTFCCTAMLITVLALVCT
LLYIKYKSRRSFIEEKKMP
AdV5 E3 CR1-beta0:
(SEQ ID NO: 218)
MTNTTNAAAATGLTSTTNTPQVSAFVNNWDNLGMWWFSIALMFVCLI
IMWLICCLKRKRARPPIYSPIIVLHPNNDGIHRLDGLKHMFFSLTV
AdV5 E3 RID-alpha:
(SEQ ID NO: 219)
MIPRVFILLTLVALFCACSTLAAVSHIEVDCIPAFTVYLLYGFVTLT
LICSLITVVIAFIQCIDWVCVRFAYLRHHPQYRDRTIAELLRIL
AdV5 E3 RID-beta:
(SEQ ID NO: 220)
MKFTVTFLLIICTLSAFCSPTSKPQRHISCRFTRIWNIPSCYNEKSD
LSEAWLYAIISVMVFCSTILALAIYPYLDIGWKRIDAMNHPTFPAPA
MLPLQQVVAGGFVPANQPRPTSPTPTEISYFNLTGGDD
AdV5 E3 14.7K:
(SEQ ID NO: 221)
MTDTLDLEMDGIITEQRLLERRRAAAEQQRMNQELQDMVNLHQCKRG
IFCLVKQAKVTYDSNTTGHRLSYKLPTKRQKLVVMVGEKPITITQHS
VETEGCIHSPCQGPEDLCTLIKTLCGLKDLIPFN
AdV5 E4 ORF6/7:
(SEQ ID NO: 222)
MTTSGVPFGMTLRPTRSRLSRRTPYSRDRLPPFETETRATILEDHPL
LPECNTLTMHNAWTSPSPPVKQPQVGQQPVAQQLDSDMNLSELPGEF
INITDERLARQETVWNITPKNMSVTHDMMLFKASRGERTVYSVCWEG
GGRLNTRVL
AdV5 E4 34K:
(SEQ ID NO: 223)
MTTSGVPFGMTLRPTRSRLSRRTPYSRDRLPPFETETRATILEDHPL
LPECNTLTMHNVSYVRGLPCSVGFTLIQEWVVPWDMVLTREELVILR
KCMHVCLCCANIDIMTSMMIHGYESWALHCHCSSPGSLQCIAGGQVL
ASWFRMVVDGAMFNQRFIWYREVVNYNMPKEVMFMSSVFMRGRHLIY
LRLWYDGHVGSVVPAMSFGYSALHCGILNNIVVLCCSYCADLSEIRV
RCCARRTRRLMLRAVRIIAEETTAMLYSCRTERRRQQFIRALLQHHR
PILMHDYDSTPM
AdV5 E4 ORF4:
(SEQ ID NO: 224)
MVLPALPAPPVCDSQNECVGWLGVAYSAVVDVIRAAAHEGVYIEPEA
RGRLDALREWIYYNYYTERSKRRDRRRRSVCHARTWFCFRKYDYVRR
SIWHDTTTNTISVVSAHSVQ
AdV5 E4 ORF3:
(SEQ ID NO: 225)
MIRCLRLKVEGALEQIFTMAGLNIRDLLRDILRRWRDENYLGMVEGA
GMFIEEIHPEGFSLYVHLDVRAVCLLEAIVQHLTNAIICSLAVEFDH
ATGGERVHLIDLHFEVLDNLLE
AdV5 E4 ORFB:
(SEQ ID NO: 226)
MFERKMVSFSVVVPELTCLYLHEHDYDVLSFLREALPDFLSSTLHFI
SPPMQQAYIGATLVSIAPSMRVIISVGSFVMVPGGEVAALVRADLHD
YVQLALRRDLRDRGIFVNVPLLNLIQVCEEPEFLQS
AdV5 E4 ORF1:
(SEQ ID NO: 227)
MAAAVEALYVVLEREGAILPRQEGFSGVYVFFSPINFVIPPMGAVML
SLRLRVCIPPGYFGRFLALTDVNQPDVFTESYIMTPDMTEELSVVLF
NHGDQFFYGHAGMAVVRLMLIRVVFPVVRQASNV

In some embodiments, the helper sequences include AdV2 helper sequences. An exemplary AdV2 genome is disclosed in the art as NCBI Reference Sequence AC_000007.1 (disclosed as SEQ ID NO:2 of PCT Patent Application Publication No. WO/2022/079429 A1). In some embodiments, a host cell disclosed herein comprises a portion of an AdV2 genome encoding for one or more helper protein sequences (e.g., E1a, E1b, E2a, E4) or RNA sequences (e.g., VA). In some embodiments, a host cell disclosed herein comprises a nucleic acid sequence encoding one or more AdV2 helper protein sequences. Certain exemplary AdV2 helper protein sequences are provided below.

AdV2 E1A:
(SEQ ID NO: 228)
MRHIICHGGVITEEMAASLLDQLIEEVLADNLPPPSHFEPPTLHELY
DLDVTAPEDPNEEAVSQIFPESVMLAVQEGIDLFTFPPAPGSPEPPH
LSRQPEQPEQRALGPVSMPNLVPEVIDLTCHEAGFPPSDDEDEEGEE
FVLDYVEHPGHGCRSCHYHRRNTGDPDIMCSLCYMRTCGMFVYSPVS
EPEPEPEPEPEPARPTRRPKLVPAILRRPTSPVSRECNSSTDSCDSG
PSNTPPEIHPVVPLCPIKPVAVRVGGRRQAVECIEDLLNESGQPLDL
SCKRPRP
AdV2 E1B 19K:
(SEQ ID NO: 229)
MEAWECLEDFSAVRNLLEQSSNSTSWFWRFLWGSSQAKLVCRIKEDY
KWEFEELLKSCGELFDSLNLGHQALFQEKVIKTLDFSTPGRAAAAVA
FLSFIKDKWSEETHLSGGYLLDFLAMHLWRAVVRHKNRLLLLSSVRP
AIIPTEEQQQEEARRRRRQEQSPWNPRAGLDPRE
AdV2 E1B 55K:
(SEQ ID NO: 230)
MERRNPSERGVPAGFSGHASVESGGETQESPATVVFRPPGNNTDGGA
TAGGSQAAAAAGAEPMEPESRPGPSGMNVVQVAELFPELRRILTINE
DGQGLKGVKRERGASEATEEARNLTFSLMTRHRPECVTFQQIKDNCA
NELDLLAQKYSIEQLTTYWLQPGDDFEEAIRVYAKVALRPDCKYKIS
KLVNIRNCCYISGNGAEVEIDTEDRVAFRCSMINMWPGVLGMDGVVI
MNVRFTGPNFSGTVFLANTNLILHGVSFYGFNNTCVEAWTDVRVRGC
AFYCCWKGVVCRPKSRASIKKCLFERCTLGILSEGNSRVRHNVASDC
GCFMLVKSVAVIKHNMVCGNCEDRASQMLTCSDGNCHLLKTIHVASH
SRKAWPVFEHNILTRCSLHLGNRRGVFLPYQCNLSHTKILLEPESMS
KVNLNGVFDMTMKIWKVLRYDETRTRCRPCECGGKHIRNQPVMLDVT
EELRPDHLVLACTRAEFGSSDEDTD
AdV2 E3 12.5K:
(SEQ ID NO: 231)
MTSGEAERLRLTHLDHCRRHKCFARGSGEFCYFELPEEHIEGPAHGV
RLTTQVELTRSLIREFTKRPLLVERERGPCVLTVVCNCPNPGLHQDL
CCHLCAEYNKYRN
AdV2 E3 CR1-alphap0:
(SEQ ID NO: 232)
MSNSSNSTSLSNFSGIGVGVILTLVILFILILALLCLRVAACCTHVC
TYCQLFKRWGQHPR
AdV2 E3 gp19K:
(SEQ ID NO: 233)
MRYMILGLLALAAVCSAAKKVEFKEPACNVTFKSEANECTTLIKCTT
EHEKLIIRHKDKIGKYAVYAIWQPGDTNDYNVTVFQGENRKTFMYKF
PFYEMCDITMYMSKQYKLWPPQKCLENTGTFCSTALLITALALVCTL
LYLKYKSRRSFIDEKKMP
AdV2 E3 CR1-beta0:
(SEQ ID NO: 234)
MTGSTIAPTTDYRNTTATGLTSALNLPQVHAFVNDWASLDMWWFSIA
LMFVCLIIMWLICCLKRRRARPPIYRPIIVLNPHNEKIHRLDGLKPC
SLLLQYD
AdV2 E3 RID alpha:
(SEQ ID NO: 235)
MIPRVLILLTLVALFCACSTLAAVAHIEVDCIPPFTVYLLYGFVTLI
LICSLVTVVIAFIQFIDWVCVRIAYLRHHPQYRDRTIADLLRIL
AdV2 E3 RID beta:
(SEQ ID NO: 236)
MKRSVIFVLLIFCALPVLCSQTSAPPKRHISCRFTQIWNIPSCYNKQ
SDLSEAWLYAIISVMVFCSTIFALAIYPYLDIGWNAIDAMNHPTFPV
PAVIPLQQVIAPINQPRPPSPTPTEISYFNLTGGDD
AdV2 E3 14.7K:
(SEQ ID NO: 237)
MTESLDLELDGINTEQRLLERRKAASERERLKQEVEDMVNLHQCKRG
IFCVVKQAKLTYEKTTTGNRLSYKLPTQRQKLVLMVGEKPITVTQHS
AETEGCLHFPYQGPEDLCTLIKTMCGIRDLIPFN
AdV2 E4 ORF6/7:
(SEQ ID NO: 238)
MTTSGVPFGMTLRPTRSRLSRRTPYSRDRLPPFETETRATILEDHPL
LPECNTLTMHNAWTSPSPPVEQPQVGQQPVAQQLDSDMNLSELPGEF
INITDERLARQETVWNITPKNMSVTHDMMLFKASRGERTVYSVCWEG
GGRLNTRVL
AdV2 E4 34K:
(SEQ ID NO: 223)
MTTSGVPFGMTLRPTRSRLSRRTPYSRDRLPPFETETRATILEDHPL
LPECNTLTMHNVSYVRGLPCSVGFTLIQEWVVPWDMVLTREELVILR
KCMHVCLCCANIDIMTSMMIHGYESWALHCHCSSPGSLQCIAGGQVL
ASWFRMVVDGAMFNQRFIWYREVVNYNMPKEVMFMSSVFMRGRHLIY
LRLWYDGHVGSVVPAMSFGYSALHCGILNNIVVLCCSYCADLSEIRV
RCCARRTRRLMLRAVRIIAEETTAMLYSCRTERRRQQFIRALLQHHR
PILMHDYDSTPM
AdV2 E4 ORF4:
(SEQ ID NO: 239)
MVLPALPAPPVCDSQNECVGWLGVAYSAVVDVIRAAAHEGVYIEPEA
RGRLDALREWIYYNYYTERAKRRDRRRRSVCHARTWFCFRKYDYVRR
SIWHDTTTNTISVVSAHSVQ
AdV2 E4 ORF3:
(SEQ ID NO: 240)
MIRCLRLKVEGALEQIFTMAGLNIRDLLRDILIRWRDENYLGMVEGA
GMFIEEIHPEGFSLYVHLDVRAVCLLEAIVQHLTNAIICSLAVEFDH
ATGGERVHLIDLHFEVLDNLLE
AdV2 E4 ORF2:
(SEQ ID NO: 241)
MFERKMVSFSVVVPELTCLYLHEHDYDVLAFLREALPDFLSSTLHFI
SPPMQQAYIGATLVSIAPSMRVIISVGSFVMVPGGEVAALVRADLHD
YVQLALRRDLRDRGIFVNVPLLNLIQVCEEPEFLQS
AdV2 E4 ORF1:
(SEQ ID NO: 227)
MAAAVEALYVVLEREGAILPRQEGFSGVYVFFSPINFVIPPMGAVML
SLRLRVCIPPGYFGRFLALTDVNQPDVFTESYIMTPDMTEELSVVLF
NHGDQFFYGHAGMAVVRLMLIRVVFPVVRQASNV

Additional AAV helper sequences are recognized in the art and include, for example, those described in U.S. Patent Application Publication Nos. 2004/0248288 A1 and 2022/0259572A1, PCT Patent Application Publication Nos. WO/1997/017458 A1, WO/2024/143429 A1, and WO/2020/208379 A1, each of which is incorporated herein by reference.

Expression control sequences include efficient RNA processing signals such as splicing and polyadenylation (polyA) signals; appropriate transcription initiation, termination, promoter and enhancer sequences; sequences that stabilize cytoplasmic mRNA; sequences that enhance protein stability; sequences that enhance translation efficiency (e.g., Kozak consensus sequence); and in some embodiments, sequences that enhance secretion of the encoded transgene product. Expression control sequences, including promoters which are native, constitutive, inducible and/or tissue-specific, are known in the art and can be utilized with the compositions and methods disclosed herein.

In some embodiments, the native promoter for the transgene is used. Without wishing to be bound by theory, the native promoter can mimic native expression of the transgene, or provide temporal, developmental, or tissue-specific expression, or expression in response to specific transcriptional stimuli. In some embodiments, the transgene is operably linked to other native expression control elements, such as enhancer elements, polyadenylation sites or Kozak consensus sequences, e.g., to mimic the native expression.

In some embodiments, the transgene is operably linked to a tissue-specific promoter, e.g., a promoter active specifically in one or more CNS or muscle cell types.

In some embodiments, a vector, e.g., a plasmid, carrying a transgene includes a selectable marker or a reporter gene. Such selectable reporters or marker genes can be used to signal the presence of the vector, e.g., plasmid, in bacterial cells. Other components of the vector, e.g., plasmid, include an origin of replication. Selection of these and other promoters and vector elements are conventional and many such sequences are available (see, e.g., Sambrook et al, Molecular Cloning: A Laboratory Manual, Cold Spring Harbor Press, Cold Spring Harbor, NY, and references cited therein).

In some embodiments, an insect cell may be used in production of the compositions described herein or in the methods of making a dependoparvovirus particle described herein. For example, an insect cell line used can be from Spodoptera frugiperda, such as Sf9, SF21, SF900+, drosophila cell lines, mosquito cell lines, e.g., Aedes albopictus derived cell lines, domestic silkworm cell lines, e.g., Bombyxmori cell lines, Trichoplusia ni cell lines such as High Five cells or Lepidoptera cell lines such as Ascalapha odorata cell lines. In some embodiments, the insect cells are susceptible to baculovirus infection, including High Five, Sf9, Se301, SeIZD2109, SeUCR1, SP900+, Sf21, BTI-TN-5B1-4, MG-1, Tn368, HzAml, BM-N, Ha2302, Hz2E5 and Ao38. Use of insect cells for expression of heterologous proteins is well recognized in the art, as are methods of introducing nucleic acids, such as vectors, e.g., insect-cell compatible vectors, into such cells and methods of maintaining such cells in culture. See, for example, O'Reilly et al., 1994, Baculovirus Expression Vectors, A Laboratory Manual. Oxford Univ. Press; Satnulski et al., 1989, J. Vir.63:3822-8; Kajigaya et al., 1991 PNAS 88:4646-50; Ruffin et al., 1992, J. Vir. 66:6922-30; Kimbauer et al., 1996, Vir.21.9:37-44; Zhao et al., 2000, Vir.272:382-93; and U.S. Pat. No. 6,204,059, the contents of each of which are incorporated herein by reference in their entireties.

In certain embodiments, insect host cell systems, in combination with baculoviral systems (e.g., as described by Luckow et al., 1988, Bio/Technology 6:47) is used. In certain embodiments, the expression system is a Trichoplusia ni, Tn 5B1-4 insect cells/baculoviral system, which can be used for production of high levels of proteins, as described in U.S. Pat. No. 6,660,521, incorporated herein by reference in its entirety.

Expansion, culture, transfection, infection, and storage of insect cells can be carried out in any cell culture media, cell transfection media or storage media known in the art. Nonlimiting examples of media are Hyclone SFX Insect Cell Culture Media, Expression System ESF AF Insect Cell Culture Medium, Basal IPL-41 Insect Cell Culture Media, ThermoFisher Sf90011 media, ThermoFisher Sf900111 media, and ThermoFisher Grace's Insect Media. Insect cell mixtures and/or media can also comprise appropriate formulation additives or elements, including but not limited to salts, acids, bases, buffers, and surfactants (such as Poloxatner 188/Pluronic F-68).

In some embodiments, the methods of the disclosure can be carried out with a mammalian cell type which allows for replication of dependoparvovirus or production of biologic products, and which can be maintained in culture. Host cells include cells derived from mammalian species including but not limited to, human, monkey, mouse, rat, rabbit, and hamster. Host cells can be of any suitable cell type, including but not limited to cell lines, fibroblasts, hepatocytes, tumor cells, and transformed cells. The mammalian cells used can be HEK293, HEK293T, HeLa, CHO, NSO, SP2/0, PER.C6, Vero, RD, BHK, HT 1080, A549, Cos-7, ARPE-19, MRC-5, WEH1, 3T3, 1.0T1/2, MDCK, COS 1, COS 7, BSC 1, BSC 40, BMT 10, W138, Saos, C2C12, HepG2, L cells, primary fibroblast, hepatocyte and myoblast cells derived from mammals, COS cells, C127, 3T3, CHO, HeLa cells, KB cells, BHK, and other mammalian cell lines as described in U.S. Pat. Nos. 6,156,303, 5,387,484, 5,741,683, 5,691,176, 6,428,988 and 5,688,676, 6,541,258, the contents of each of which are incorporated herein by reference.

In some embodiments, the host cell comprises a nucleic acid encoding a variant capsid polypeptide disclosed herein, where the nucleic acid is integrated into the host cell genome. Such host cells include adenovirus rep and cap genes integrated into the genome. Transcription of the integrated rep and cap genes may be dependent upon introduction of certain helper virus sequences (e.g., adenovirus E4, E2a and/or VA RNA) into the cell by transduction or other suitable means. Example plasmid free host cells are described in U.S. Patent Application Publication No. 2022/0025396 A1, and U.S. Pat. No. 5,658,785, incorporated herein by reference.

In some embodiments, the host cells are trans-complementing packaging cell lines that provide functions deleted from a replication-defective helper virus, e.g., HEK293 cells or other Ea trans-complementing cells. In some embodiments, the packaging cell line 293-10-3 is used as described in U.S. Pat. No. 6,281,010, incorporated herein by reference.

In some embodiments, mammalian host cells (e.g. 293T cells) can be in an adherent state (e.g., adhered/attached to a suitable surface of a cell culture flask, vial, tray, well, tube, etc.). In other embodiments, mammalian host cells can be in a suspended state (e.g., suspended in a medium).

### 6.6.2. Viral Particle Production

In some embodiments, the nucleic acids of the disclosure are situated as a part of any genetic element (vector) which can be delivered to a host cell, e.g., naked DNA, a plasmid, phage, transposon, cosmid, episome, a protein in a non-viral delivery vehicle (e.g., a lipid-based carrier), virus, etc., which transfer the sequences carried thereon. Such a vector can be delivered into a host cell by any suitable method, including transfection, liposome delivery, electroporation, membrane fusion techniques, viral infection, high velocity DNA-coated pellets, and protoplast fusion. A person of skill in the art possesses the knowledge and skill in nucleic acid manipulation to construct any embodiment of this invention and said skills include genetic engineering, recombinant engineering, and synthetic techniques. See, e.g., Sambrook et al, Molecular Cloning: A Laboratory Manual, Cold Spring Harbor Press, Cold Spring Harbor, NY.

In some embodiments, a vector of the disclosure comprises sequences encoding a dependoparvovirus variant capsid polypeptide as provided for herein or a fragment thereof. In some embodiments, a vector of the disclosure comprises sequences encoding a dependoparvovirus Rep protein or a fragment thereof. Such a Rep coding region encodes at least for AAV Rep78, Rep68, Rep52, and Rep40, or functional homologs thereof. The Rep coding region is not required to include all wild-type genes but may be altered (e.g., by insertion, deletion, or mutation of one or more nucleotides) so long as the rep genes present provide for sufficient replication functions when expressed in the recombinant cell. The Rep coding region may be derived from any AAV serotype. In some embodiments, the Rep coding region is or comprises a rep gene encoding AAV2 Rep proteins, exemplary sequences of which are provided below.

AAV2 Rep78:
(SEQ ID NO: 242)
MPGFYEIVIKVPSDLDGHLPGISDSFVNWVAEKEWELPPDSDMDLNL
IEQAPLTVAEKLQRDFLTEWRRVSKAPEALFFVQFEKGESYFHMHVL
VETTGVKSMVLGRFLSQIREKLIQRIYRGIEPTLPNWFAVTKTRNGA
GGGNKVVDECYIPNYLLPKTQPELQWAWTNMEQYLSACLNLTERKRL
VAQHLTHVSQTQEQNKENQNPNSDAPVIRSKTSARYMELVGWLVDKG
ITSEKQWIQEDQASYISFNAASNSRSQIKAALDNAGKIMSLTKTAPD
YLVGQQPVEDISSNRIYKILELNGYDPQYAASVFLGWATKKFGKRNT
IWLFGPATTGKTNIAEAIAHTVPFYGCVNWTNENFPFNDCVDKMVIW
WEEGKMTAKVVESAKAILGGSKVRVDQKCKSSAQIDPTPVIVTSNTN
MCAVIDGNSTTFEHQQPLQDRMFKFELTRRLDHDFGKVTKQEVKDFF
RWAKDHVVEVEHEFYVKKGGAKKRPAPSDADISEPKRVRESVAQPST
SDAEASINYADRYQNKCSRHVGMNLMLFPCRQCERMNQNSNICFTHG
QKDCLECFPVSESQPVSVVKKAYQKLCYIHHIMGKVPDACTACDLVN
VDLDDCIFEQ
AAV2 Rep68:
(SEQ ID NO: 243)
MPGFYEIVIKVPSDLDGHLPGISDSFVNWVAEKEWELPPDSDMDLNL
IEQAPLTVAEKLQRDFLTEWRRVSKAPEALFFVQFEKGESYFHMHVL
VETTGVKSMVLGRFLSQIREKLIQRIYRGIEPTLPNWFAVTKTRNGA
GGGNKVVDECYIPNYLLPKTQPELQWAWTNMEQYLSACLNLTERKRL
VAQHLTHVSQTQEQNKENQNPNSDAPVIRSKTSARYMELVGWLVDKG
ITSEKQWIQEDQASYISFNAASNSRSQIKAALDNAGKIMSLTKTAPD
YLVGQQPVEDISSNRIYKILELNGYDPQYAASVFLGWATKKFGKRNT
IWLFGPATTGKTNIAEAIAHTVPFYGCVNWTNENFPFNDCVDKMVIW
WEEGKMTAKVVESAKAILGGSKVRVDQKCKSSAQIDPTPVIVTSNTN
MCAVIDGNSTTFEHQQPLQDRMFKFELTRRLDHDFGKVTKQEVKDFF
RWAKDHVVEVEHEFYVKKGGAKKRPAPSDADISEPKRVRESVAQPST
SDAEASINYADRLARGHSL
AAV2 Rep52:
(SEQ ID NO: 244)
MELVGWLVDKGITSEKQWIQEDQASYISFNAASNSRSQIKAALDNAG
KIMSLTKTAPDYLVGQQPVEDISSNRIYKILELNGYDPQYAASVFLG
WATKKFGKRNTIWLFGPATTGKTNIAEAIAHTVPFYGCVNWTNENFP
FNDCVDKMVIWWEEGKMTAKVVESAKAILGGSKVRVDQKCKSSAQID
PTPVIVTSNTNMCAVIDGNSTTFEHQQPLQDRMFKFELTRRLDHDFG
KVTKQEVKDFFRWAKDHVVEVEHEFYVKKGGAKKRPAPSDADISEPK
RVRESVAQPSTSDAEASINYADRYQNKCSRHVGMNLMLFPCRQCERM
NQNSNICFTHGQKDCLECFPVSESQPVSVVKKAYQKLCYIHHIMGKV
PDACTACDLVNVDLDDCIFEQ
AAV2 Rep40:
(SEQ ID NO: 245)
MELVGWLVDKGITSEKQWIQEDQASYISFNAASNSRSQIKAALDNAG
KIMSLTKTAPDYLVGQQPVEDISSNRIYKILELNGYDPQYAASVFLG
WATKKFGKRNTIWLFGPATTGKTNIAEAIAHTVPFYGCVNWTNENFP
FNDCVDKMVIWWEEGKMTAKVVESAKAILGGSKVRVDQKCKSSAQID
PTPVIVTSNTNMCAVIDGNSTTFEHQQPLQDRMFKFELTRRLDHDFG
KVTKQEVKDFFRWAKDHVVEVEHEFYVKKGGAKKRPAPSDADISEPK
RVRESVAQPSTSDAEASINYADRLARGHSL

In some embodiments the Rep coding region encodes for Rep sequences having at least 80%, at least 85%, at least 90%, at least 95%, or at least 99% sequence identity to one or more AAV2 Rep proteins. In some embodiments, the Rep coding region comprises a nucleotide sequence encoding for a Rep78 protein having at least 80%, at least about 85%, at least about 90%, at least about 95%, at least 98%, at least 99%, or 100% sequence identity to AAV2 Rep78. In some embodiments, the Rep coding region comprises a nucleotide sequence encoding for a Rep68 protein having at least 80%, at least about 85%, at least about 90%, at least about 95%, at least 98%, at least 99%, or 100% sequence identity to AAV2 Rep68. In some embodiments, the Rep coding region comprises a nucleotide sequence encoding for a Rep52 protein having at least 80%, at least about 85%, at least about 90%, at least about 95%, at least 98%, at least 99%, or 100% sequence identity to AAV2 Rep52. In some embodiments, the Rep coding region comprises a nucleotide sequence encoding for a Rep40 protein having at least 80%, at least about 85%, at least about 90%, at least about 95%, at least 98%, at least 99%, or 100% sequence identity to AAV2 Rep40.

In some embodiments, the Rep coding sequence encodes for wild type Rep proteins. Alternatively, the Rep coding sequence may encode for one or more mutant Rep proteins having improved properties compared with wild type Rep proteins. Examples of such mutant Rep proteins are described in U.S. Pat. No. 11,060,070, incorporated herein by reference.

Additional exemplary Rep gene and protein sequences are disclosed as SEQ ID NOs: 7 to 15 and 20 to 22 of PCT Patent Application Publication No. WO/2022/079429 A1, incorporated herein by reference.

In some embodiments, such vectors contain both dependoparvovirus cap and rep proteins. In vectors in which both AAV rep and cap are provided, in some embodiments, the dependoparvovirus rep and dependoparvovirus cap sequences are both of the same dependoparvovirus species or serotype origin. Alternatively, the present embodiments also provide vectors in which the rep sequences are from a dependoparvovirus species or serotype which differs from that which is providing the cap sequences (e.g., AAV2 rep sequences and AAV9 cap sequences). In some embodiments, the rep and cap sequences are expressed from separate sources (e.g., separate vectors, or a host cell genome and a vector). In some embodiments, the rep sequences are fused in frame to cap sequences of a different dependoparvovirus species or serotype to form a chimeric dependoparvovirus vector.

In some embodiments, a vector of the disclosure comprises one or more helper sequences. Examples of helper sequences include E1a, E1b, E2a, E4, and VA. Certain exemplary helper protein sequences are provided in Section 6.6.1. Additional AAV helper sequences are recognized in the art and include, for example, those described in U.S. Patent Application Publication Nos. 2004/0248288 A1 and 2022/0259572A1, and in PCT Patent Application Publication Nos. WO/1997/017458 A1, WO/2024/143429 A1, and WO/2020/208379 A1, each of which is incorporated herein by reference.

In some embodiments, the vectors of the disclosure further contain a payload, e.g., a minigene comprising a selected transgene, e.g., flanked by dependoparvovirus 5′ ITR and dependoparvovirus 3′ ITR. In some embodiments, the ITR is from the same serotype as the variant capsid polypeptide. In some embodiments, the ITR is of a different serotype than the variant capsid polypeptide. In some embodiments, the viral genome comprises two ITR sequence regions, wherein the ITRs are of the same serotype as one another. In some embodiments, the viral genome comprises two ITR sequence regions, wherein the ITRs are of different serotypes. Non-limiting examples include zero, one or both of the ITRs having the same serotype as the capsid. In one embodiment both ITRs of the viral genome of the AAV particle are AAV2 ITRs. Independently, each ITR may be about 100 to about 150 nucleotides in length. An ITR may be about 100-105 nucleotides in length, 106-110 nucleotides in length, 111-115 nucleotides in length, 116-120 nucleotides in length, 121-125 nucleotides in length, 126-130 nucleotides in length, 131-135 nucleotides in length, 136-140 nucleotides in length, 141-145 nucleotides in length or 146-150 nucleotides in length. In one embodiment, the ITRs are 140-142 nucleotides in length. Nonlimiting examples of ITR lengths are 102, 105, 130, 140, 141, 142, 145 nucleotides in length.

The vectors described herein, e.g., a plasmid, are useful for a variety of purposes, but are particularly well suited for use in production of recombinant dependoparvovirus particles comprising dependoparvovirus sequences or a fragment thereof, and in some embodiments, a payload.

In one aspect, the disclosure provides a method of making a dependoparvovirus particle (e.g., a dependoparvovirus B particle, e.g., an AAV9 particle), or a portion thereof. In some embodiments, the method comprises culturing a host cell which contains a nucleic acid sequence encoding a dependoparvovirus variant capsid protein as provided for herein, or fragment thereof; a functional rep gene (e.g., encoding Rep proteins as described herein); a payload, e.g., a minigene comprising dependoparvovirus inverted terminal repeats (ITRs) and a transgene; and sufficient helper functions to promote packaging of the payload, e.g., minigene, into the dependoparvovirus capsid. In some embodiments, the components necessary to be cultured in the host cell to package a payload, e.g., minigene, in a dependoparvovirus capsid are provided to the host cell in trans. In some embodiments, any one or more of the required components (e.g., payload (e.g., minigene), rep sequences, cap sequences, and/or helper functions) are provided by a host cell which has been engineered to stably comprise one or more of the required components using methods known to those of skill in the art. In some embodiments, a host cell which has been engineered to stably comprise the required component(s) comprises it under the control of an inducible promoter. In some embodiments, the required components are under the control of a constitutive promoter. Examples of suitable inducible and constitutive promoters are provided herein, and further examples are known to those of skill in the art. In some embodiments, a selected host cell which has been engineered to stably comprise one or more components comprises a component under the control of a constitutive promoter and another component under the control of one or more inducible promoters. For example, a host cell which has been engineered to stably comprise the required components is generated from HEK 293 cells (e.g., which comprise helper functions under the control of a constitutive promoter), which comprises the rep and/or cap proteins under the control of one or more inducible promoters.

In some embodiments, the payload (e.g., minigene), rep sequences, cap sequences, and helper functions required for producing a dependoparvovirus particle of the disclosure are delivered to the packaging host cell in the form of any genetic element which transfers the sequences carried thereon (e.g., in a vector or combination of vectors). The genetic element may be delivered by any suitable method, including those described herein. Methods used to construct genetic elements, vectors, and other nucleic acids of the disclosure are known to those with skill and include genetic engineering, recombinant engineering, and synthetic techniques. See, e.g., Sambrook et al, Molecular Cloning: A Laboratory Manual, Cold Spring Harbor Press, Cold Spring Harbor, NY Similarly, methods of generating rAAV virions are well known and the selection of a suitable method is not a limitation on the present invention. See, e.g., K. Fisher et al, J. Virol, 70:520-532 (1993) and U.S. Pat. No. 5,478,745. Unless otherwise specified, the dependoparvovirus ITRs, and other selected dependoparvovirus components described herein, are readily selected from among any dependoparvovirus species and serotypes, e.g., AAV1, AAV2, AAV3, AAV4, AAV5, AAV6, AAV7, AAV8, AAVrh74, or AAV9.

ITRs or other dependoparvovirus components may be readily isolated using techniques available to those of skill in the art from a dependoparvovirus species or serotype. Dependoparvovirus species and serotypes may be isolated or obtained from academic, commercial, or public sources (e.g., the American Type Culture Collection, Manassas, VA). In some embodiments, the dependoparvovirus sequences may be obtained through synthetic or other suitable means by reference to published sequences such as are available in the literature or in databases such as, e.g., GenBank or PubMed.

Methods of expressing proteins (e.g., recombinant or heterologous proteins, e.g., dependoparvovirus polypeptides) in insect cells are well documented, as are methods of introducing nucleic acids, such as vectors, e.g., insect-cell compatible vectors, into such cells and methods of maintaining such cells in culture. See, for example, METHODS IN MOLECULAR BIOLOGY, ed. Richard, Humana Press, N J (1995); O'Reilly et al., BACULOVIRUS EXPRESSION VECTORS, A LABORATORY MANUAL, Oxford Univ. Press (1994); Samulski et al., J. Vir. 63:3822-8 (1989); Kajigaya et al., Proc. Nat'l. Acad. Sci. USA 88:4646-50 (1991); Ruffing et al., J. Vir. 66:6922-30 (1992); Kirnbauer et al., Vir. 219:37-44 (1996); Zhao et al., Vir. 272:382-93 (2000); Samulski et al., and U.S. Pat. No. 6,204,059. In some embodiments, a nucleic acid construct encoding dependoparvovirus polypeptides (e.g., a dependoparvovirus genome) in insect cells is an insect cell-compatible vector. An “insect cell-compatible vector” as used herein refers to a nucleic acid molecule capable of productive transformation or transfection of an insect or insect cell. Exemplary biological vectors include plasmids, linear nucleic acid molecules, and recombinant viruses. Any vector can be employed as long as it is insect cell compatible. The vector may integrate into the insect cell's genome or remain present extra-chromosomally. The vector may be present permanently or transiently, e.g., as an episomal vector. Vectors may be introduced by any means known in the art. Such means include but are not limited to chemical treatment of the cells, electroporation, or infection. In some embodiments, the vector is a baculovirus, a viral vector, or a plasmid. Methods of dependoparvovirus capsid (e.g., AAV) production in insect cells include, for example, those described in U.S. Patent Application Publication No. 2024/0093231 A1, U.S. Pat. No. 11,306,291, Joshi et al., 2024, Methods Mol Biol, 2829:203-214, and Marwidi et al., 2024, Mol Ther Methods Clin Dev., 32(2)101228, incorporated herein by reference.

In some embodiments, a nucleic acid sequence encoding a dependoparvovirus polypeptide is operably linked to regulatory expression control sequences for expression in a specific cell type, such as Sf9 or HEK cells. Techniques known to one skilled in the art for expressing foreign genes in insect host cells or mammalian host cells can be used with the compositions and methods of the disclosure. Methods for molecular engineering and expression of polypeptides in insect cells are described, for example, in Summers and Smith. A Manual of Methods for Baculovirus Vectors and Insect Culture Procedures, Texas Agricultural Experimental Station Bull. No. 7555, College Station, Tex. (1986); Luckow. 1991. In Prokop et al., Cloning and Expression of Heterologous Genes in Insect Cells with Baculovirus Vectors' Recombinant DNA Technology and Applications, 97-152 (1986); King, L. A. and R. D. Possee, The baculovirus expression system, Chapman and Hall, United Kingdom (1992); O'Reilly, D. R., L. K. Miller, V. A. Luckow, Baculovirus Expression Vectors: A Laboratory Manual, New York (1992); W. H. Freeman and Richardson, C. D., Baculovirus Expression Protocols, Methods in Molecular Biology, volume 39 (1995); U.S. Pat. No. 4,745,051; US2003148506; and WO 03/074714. Promoters suitable for transcription of a nucleotide sequence encoding a dependoparvovirus polypeptide include the polyhedron, p10, p35 or IE-1 promoters and further promoters described in the above references are also contemplated.

In some embodiments, providing a cell comprising a nucleic acid described herein comprises acquiring a cell comprising the nucleic acid.

Methods of cultivating host cells, cell-free systems, and other translation systems are known to those of skill in the art. In some embodiments, cultivating a host cell comprises providing the cell with suitable media and incubating the cell and media for a time suitable to achieve viral particle production.

In some embodiments, a method of making a dependoparvovirus particle further comprises a purification step comprising isolating the dependoparvovirus particle from one or more other components (e.g., from a cell or media component).

In some embodiments, production of the dependoparvovirus particle comprises one or more (e.g., all) of: expression of dependoparvovirus polypeptides, assembly of a dependoparvovirus capsid, expression (e.g., duplication) of a dependoparvovirus genome, and packaging of the dependoparvovirus genome into the dependoparvovirus capsid to produce a dependoparvovirus particle. In some embodiments, production of the dependoparvovirus particle further comprises secretion of the dependoparvovirus particle into the media. The dependoparvovirus particle can be isolated from the collected media. In other embodiments, dependoparvovirus particles are isolated from host cells. For instance, adherent host cells can subsequently be collected by scraping and/or pelleting and suspended cells can be collected by pelleting and transferred into a receptacle. Collection steps can be repeated as necessary for full collection of produced cells. If necessary, host cell lysis can be achieved by consecutive freeze-thaw cycles (−80° C. to 37° C.), chemical lysis (such as adding detergent, e.g., triton), mechanical lysis, or by allowing the cell culture to degrade after reaching ˜0% viability. Cellular debris can be removed by centrifugation and/or depth filtration.

In some embodiments, and as described elsewhere herein, the nucleic acid molecule encoding the variant capsid polypeptide is disposed in a dependoparvovirus genome. In some embodiments, and as described elsewhere herein, the nucleic acid molecule encoding the variant capsid polypeptide is packaged into a dependoparvovirus particle along with the dependoparvovirus genome as part of a method of making a dependoparvovirus particle described herein. In other embodiments, the nucleic acid molecule encoding the variant capsid polypeptide is not packaged into a dependoparvovirus particle made by a method described herein.

In some embodiments, a method of making a dependoparvovirus particle described herein produces a dependoparvovirus particle comprising a payload (e.g., a payload described herein) and the variant capsid polypeptide. In some embodiments, the payload comprises a second nucleic acid (e.g., in addition to the dependoparvovirus genome), and production of the dependoparvovirus particle comprises packaging the second nucleic acid into the dependoparvovirus particle. In some embodiments, a cell, cell-free system, or other translation system for use in a method of making a dependoparvovirus particle comprises the second nucleic acid. In some embodiments, the second nucleic acid comprises an exogenous sequence (e.g., exogenous to the dependoparvovirus, the cell, or to a target cell or subject who will be administered the dependoparvovirus particle). In some embodiments, the exogenous sequence encodes an exogenous polypeptide. In some embodiments, the exogenous sequence encodes a therapeutic product.

In some embodiments, virus particles of the disclosure have a similar production efficiency to viral particles with a reference capsid polypeptide, for example, with the wild-type capsid polypeptide (SEQ ID NO:7). In some embodiments, production efficiency of viral particles of the disclosure is (a) at least 0.1-fold, at least 0.2-fold, at least 0.3-fold, at least 0.4-fold, at least 0.5-fold, at least 0.6-fold, at least 0.7-fold, at least 0.8-fold, or at least 0.9-fold and/or (b) up to 1-fold, e.g., as compared to a viral particle with a reference capsid polypeptide, for example, with the wild-type capsid polypeptide (SEQ ID NO:1), or the production efficiency is within any range bounded by a value in (a) and a value in (b). In some embodiments, production efficiency is at least 0.5-fold as compared to a viral particle with the wild-type capsid polypeptide (SEQ ID NO:7).

Production efficiency can be evaluated by producing viral particles having a variant capsid with a genome encoding a unique barcode and a fluorescent reporter gene under the control of a ubiquitous (e.g., CBh), neuronal cell-type specific (e.g., hSYN), or muscle-specific promoter via transient triple transfection of adherent HEK293T cells followed by iodixanol gradient purification.

Various methods and systems for dependoparvovirus production in host cells are recognized in the art and are contemplated herein including, for example, those described in U.S. Patent Application Publication Nos. 20220064671 A1, 20220259572 A1, 20220025396 A1 and PCT Patent Application Publication Nos. WO/1999/011764 A2, WO/2023/178220 A1, WO/2020/208379 A1, WO/2023/143063 A1, WO/2023/239627 A2, WO/2021/156609 A1, and WO/2021/113767 A1, each of which is incorporated herein by reference in its entirety.

### 6.7. Applications

The disclosure is directed, in part, to compositions comprising a nucleic acid, polypeptide, or particles described herein. The disclosure is further directed, in part, to methods utilizing a composition, nucleic acid, polypeptide, or particles described herein. As will be apparent based on the disclosure, nucleic acids, polypeptides, particles, and methods disclosed herein have a variety of utilities.

The disclosure is directed, in part, to a vector comprising a nucleic acid described herein, e.g., a nucleic acid encoding a variant capsid polypeptide. Many types of vectors are known to those of skill in the art. In some embodiments, a vector comprises a plasmid. In some embodiments, the vector is an isolated vector, e.g., removed from a cell or other biological components.

The disclosure is directed, in part to a cell, cell-free system, or other translation system, comprising a nucleic acid or vector described herein, e.g., a nucleic acid or vector comprising a nucleic acid molecule encoding a variant capsid polypeptide. In some embodiments, the cell, cell-free system, or other translation system is capable of producing dependoparvovirus particles comprising the variant capsid polypeptides. In some embodiments, the cell, cell-free system, or other translation system comprises a nucleic acid comprising a dependoparvovirus genome or components of a dependoparvovirus genome sufficient to promote production of dependoparvovirus particles comprising the variant capsid polypeptides.

In some embodiments, the cell, cell-free system, or other translation system further comprises one or more non-dependoparvovirus nucleic acid sequences that promote dependoparvovirus particle production and/or secretion. Said sequences are referred to herein as helper sequences. In some embodiments, a helper sequence comprises one or more genes from another virus, e.g., an adenovirus or herpes virus. In some embodiments, the presence of a helper sequence is necessary for production and/or secretion of a dependoparvovirus particle. In some embodiments, a cell, cell-free system, or other translation system comprises a vector, e.g., plasmid, comprising one or more helper sequences.

In some embodiments, a cell, cell-free system, or other translation system comprises a first nucleic acid and a second nucleic acid, wherein the first nucleic acid comprises sequences encoding one or more dependoparvovirus genes (e.g., a Cap gene, a Rep gene, or a complete dependoparvovirus genome) and a helper sequence, and wherein the second nucleic acid comprises a payload. In some embodiments, a cell, cell-free system, or other translation system comprises a first nucleic acid and a second nucleic acid, wherein the first nucleic acid comprises sequences encoding one or more dependoparvovirus genes (e.g., a Cap gene, a Rep gene, or a complete dependoparvovirus genome) and a payload, and wherein the second nucleic acid comprises a helper sequence. In some embodiments, a cell, cell-free system, or other translation system comprises a first nucleic acid and a second nucleic acid, wherein the first nucleic acid comprises a helper sequence and a payload, and wherein the second nucleic acid comprises sequences encoding one or more dependoparvovirus genes (e.g., a Cap gene, a Rep gene, or a complete dependoparvovirus genome). In some embodiments, a cell, cell-free system, or other translation system comprises a first nucleic acid, a second nucleic acid, and a third nucleic acid, wherein the first nucleic acid comprises sequences encoding one or more dependoparvovirus genes (e.g., a Cap gene, a Rep gene, or a complete dependoparvovirus genome), the second nucleic acid comprises a helper sequence, and the third nucleic acid comprises a payload.

In some embodiments, the first nucleic acid, second nucleic acid, and optionally third nucleic acid are situated in separate molecules, e.g., separate vectors or a vector and genomic DNA. In some embodiments, one, two, or all of the first nucleic acid, second nucleic acid, and optionally third nucleic acid are integrated (e.g., stably integrated) into the genome of a cell.

In some embodiments, a cell of the disclosure is generated by transfecting a suitable cell with a nucleic acid described herein. In some embodiments, a method of making a dependoparvovirus particle comprising a variant capsid polypeptide as provided for herein or improving a method of making a dependoparvovirus particle comprises providing a cell described herein. In some embodiments, providing a cell comprises transfecting a suitable cell with one or more nucleic acids described herein.

Many types and kinds of cells suitable for use with the nucleic acids and vectors described herein are known in the art. In some embodiments, the cell is a human cell. In some embodiments, the cell is an immortalized cell or a cell from a cell line known in the art. In some embodiments, the cell is an HEK293 cell. In some embodiments, the cell is an HEK293T cell.

### 6.7.1. Methods of Delivering a Payload

The disclosure is directed, in part, to a method of delivering a payload to a cell, e.g., a cell in a subject or in a sample. In some embodiments, a method of delivering a payload to a cell comprises contacting the cell with a dependoparvovirus particle comprising a variant capsid polypeptide (e.g., described herein) comprising the payload. The disclosure also includes a dependoparvovirus particle comprising a variant capsid polypeptide (e.g., described herein) comprising a payload described herein for use in the methods of delivering a payload described herein. In some embodiments, the dependoparvovirus particle is a dependoparvovirus particle described herein and comprises a payload described herein. In some embodiments, the cell is a myocyte (muscle cell). Non-limiting examples of skeletal muscles include the biceps, the triceps, the quadriceps, the tibialis interior, the gastrocnemius muscle, and diaphragm. In some embodiments, the cell is a CNS cell. In some embodiments, the method is conducted ex vivo. In some embodiments, the cell is a cell in an ex vivo sample that has been obtained from a subject.

In some embodiments, the payload comprises a transgene. In some embodiments, the transgene is a nucleic acid sequence heterologous to the vector sequences flanking the transgene which encodes a polypeptide, RNA (e.g., a miRNA or siRNA) or other product of interest. In some embodiments, the nucleic acid of the transgene is operatively linked to a regulatory component in a manner sufficient to promote transgene transcription, translation, and/or expression in a host cell.

In aspects, a transgene is any polypeptide- or RNA-encoding sequence and the transgene selected will depend upon the use envisioned. In some embodiments, a transgene comprises a reporter sequence, which upon expression produces a detectable signal. Such reporter sequences include, without limitation, DNA sequences encoding colorimetric reporters (e.g., β-lactamase, β-galactosidase (LacZ), alkaline phosphatase), cell division reporters (e.g., thymidine kinase), fluorescent or luminescence reporters (e.g., green fluorescent protein (GFP) or luciferase), resistance conveying sequences (e.g., chloramphenicol acetyltransferase (CAT)), or membrane bound proteins including to which high affinity antibodies directed thereto exist or can be produced by conventional means, e.g., comprising an antigen tag, e.g., hemagglutinin or Myc.

In some embodiments, a reporter sequence operably linked with regulatory elements which drive their expression, provide signals detectable by conventional means, including enzymatic, radiographic, colorimetric, fluorescence or other spectrographic assays, fluorescent activating cell sorting assays and immunological assays, including enzyme linked immunosorbent assay (ELISA), radioimmunoassay (RIA) and immunohistochemistry. In some embodiments, the transgene encodes a product which is useful in biology and medicine, such as RNA, proteins, peptides, enzymes, dominant negative mutants. In some embodiments, the RNA comprises a tRNA, ribosomal RNA, dsRNA, catalytic RNAs, small hairpin RNA, siRNA, trans-splicing RNA, and antisense RNAs. In some embodiments, the RNA inhibits or abolishes expression of a targeted nucleic acid sequence in a treated subject (e.g., a human or animal subject).

In some embodiments, the transgene is used to correct or ameliorate gene deficiencies. In some embodiments, gene deficiencies include deficiencies in which normal genes are expressed at less than normal levels or deficiencies in which the functional gene product is not expressed. In some embodiments, the transgene encodes a therapeutic protein or polypeptide which is expressed in a host cell. In some embodiments, a dependoparvovirus particle comprises or delivers multiple transgenes, e.g., to correct or ameliorate a gene defect caused by a multi-subunit protein. In some embodiments, a different transgene (e.g., each situated/delivered in a different dependoparvovirus particle, or in a single dependoparvovirus particle) is used to encode each subunit of a protein, or to encode different peptides or proteins, e.g., when the size of the DNA encoding the protein subunit is large, e.g., for immunoglobulin, platelet-derived growth factor, or dystrophin protein. In some embodiments, different subunits of a protein are encoded by the same transgene, e.g., a single transgene encoding each of the subunits with the DNA for each subunit separated by an internal ribozyme entry site (IRES). In some embodiments, the DNA is separated by sequences encoding a 2A peptide, which self-cleaves in a post-translational event. See, e.g., Donnelly et al, J. Gen. Virol., 78(Pt 1):13-21 (January 1997); Furler, et al, Gene Ther., 8(11):864-873 (June 2001); Klump et al., Gene Ther8(10):811-817 (May 2001) (incorporated herein by reference in their entirety).

In some embodiments, virus particles comprising a genome are provided, wherein the genome includes a nucleic acid expression construct. The nucleic acid expression construct can include a heterologous transgene and one or more regulatory elements.

In some embodiments, the regulatory elements include a promotor, e.g., a promoter that is active in a target cell or tissue type of interest. In some embodiments, the promoter is a ubiquitous promoter. In other embodiments, the promoter is selective or specific to a target cell or tissue type (e.g., CNS and/or muscle), and, optionally, is less (or not) active in a cell or tissue type where transgene expression is not desired (e.g., liver). A promoter that is selective for a first cell or tissue type over a second cell or tissue type is active in the first cell or tissue type and less active or silent in the second cell or tissue type. In some embodiments, the promoter is a CNS-specific or CNS-selective promoter. In some embodiments, the promoter is a muscle-specific promoter-specific or muscle-selective promoter. In some embodiments, the promoter is active in both CNS and muscle tissue.

In some embodiments, the promoter is a ubiquitous or constitutive promoter active in a mammalian cell, for example a human cell, for example, in a human cell type of interest. In some embodiments, the cell type is a muscle cell such as, for example, a cardiac myocyte, skeletal muscle myocyte, or smooth muscle myocyte. In some embodiments, the cell type is a CNS cell such as, for example, a neuronal cell, a glial cell, an endothelia cell, and the like. Examples of ubiquitous promoters include, but are not limited, to a CAG promoter (hybrid from a cytomegalovirus early enhancer element, a chicken-beta actin promoter, e.g., the first exon and the first intron of the chicken beta actin gene, and the splice acceptor of the rabbit beta globin gene), chicken-beta actin promoter, CBA promoter, CBh promoter, CB6 promoter, CMV promoter, human EF1-alpha promoter, PGK promoter, ubiquitin C (UBC) promoter and fragments thereof. In some embodiments, the promoter is a tissue-specific promoter, for example, a promoter specific in CNS tissue or cells of the CNS. Examples of CNS-specific promoters include but are not limited to a synapsin (SYN or SYN1) promoter, a neuron-specific enolase (NSE) promoter, a Ca²⁺/calmodulin-dependent kinase subunit a (CaMKII) promoter, a synapsin I with a minimal CMV sequence (Synl-minCMV) promoter, a glial fibrillary acidic protein (GFAP) promoter, a internexin neuronal intermediate filament protein alpha (INA) promoter, a nestin (NES) promoter, a neurofilament light chain (NfL) promoter, a neurofilament heavy chain (NfH) promoter, a myelin-associated oligodendrocyte basic protein (MOBP) promoter, a myelin basic protein (MBP) promoter, a tyrosine hydroxylase (TH) promoter, a forkhead box A2 (FOXA2) promoter, a aldehyde dehydrogenase 1 family member L1 (ALDH1L1) promoter, a glutamate decarboxylase 2 (GAD2) promoter, a riken gene A930098C07Rik (A93) promoter, a somatostatin (SST) promoter, a platelet derived growth factor receptor alpha (PDGFRA) promoter, a glutamate receptor metabotropic 1 (GRM1) promoter, a C-type natriuretic peptide precursor (NPPC) promoter, a adrenomedullin (ADM) promoter, a type 2 lactosamine alpha-2,3-sialyltransferase (ST3GAL6) promoter, a ras responsive element binding protein 1 (RREB1) promoter, a deiodinase iodothyronine type II (DIO2) promoter, an excitatory amino acid transporter 2 (EAAT2) promoter, a nuclear receptor subfamily 2 group F member 2 (NR2F2) promoter, a platelet-derived growth factor (PDGF) promoter, a methyl-CpG binding protein 2 (MeCP2) promoter, and mouse, primate or human homologs of any of the forgoing, and fragments (e.g., active fragments) of any of the foregoing. In some embodiments, the CNS-specific promoter is a neuron specific promoter. In some embodiments, the CNS-specific promoter is an astrocyte-specific promoter. In some embodiments, the promoter is a promoter specific in muscle tissue or muscle cells, e.g., a desmin, MCK, TNNT2, or smooth muscle 22 (SM22) promoter. Further exemplary muscle specific promoters are described below.

In some embodiments, the promoter is a CBh promoter. An exemplary CBh promoter sequence is set forth as SEQ ID NO:100. In some embodiments, the CBh promoter comprises a nucleotide sequence having at least 90%, at least 95%, at least 96%, at least 97%, or at least 98%, at least 99%, or 100% sequence identity to SEQ ID NO:100.

In other embodiments, the promoter is a EF1a promoter, for example a human EF1a promoter. An exemplary human EF1a promoter sequence is set forth as SEQ ID NO:101. In some embodiments, the human EF1a promoter comprises a nucleotide sequence having at least 90%, at least 95%, at least 96%, at least 97%, or at least 98%, at least 99%, or 100% sequence identity to SEQ ID NO:101.

In other embodiments, the promoter is a synapsin promoter, for example a human synapsin (hSYN) promoter. An exemplary hSYN promoter sequence is set forth as SEQ ID NO:102. In some embodiments, the hSYN promoter comprises a nucleotide sequence having at least 90%, at least 95%, at least 96%, at least 97%, or at least 98%, at least 99%, or 100% sequence identity to SEQ ID NO:102.

In some embodiments, where preferential expression of a payload (e.g., a transgene) to a specific tissue is desired, the promoter is a tissue-specific promoter. Example tissue-specific promoters include muscle-specific promoters.

In some embodiments, the promoter is a promoter specific in muscle tissue or muscle cells, e.g., a desmin, MCK, TNNT2, or smooth muscle 22 (SM22) promoter. Further exemplary muscle specific promoters are described below.

In some embodiments, the nucleic acid expression construct comprises an intron. In some embodiments, the intron is disposed between the promoter and the heterologous transgene. In some aspects, the intron is disposed 5′ to the heterologous transgene on the expression construct, for example immediately 5′ to the heterologous transgene or 100 nucleotides or less 5′ to the heterologous transgene. In some aspects, the intron is a chimeric intron derived from human b-globin and Ig heavy chain (also known as b-globin splice donor/immunoglobulin heavy chain splice acceptor intron, or b-globin/IgG chimeric intron; Reed, R., et al. Genes and Development, 1989, incorporated herein by reference in its entirety). In other aspects, the intron is a VH4 intron or a SV40 intron.

As provided herein, in some embodiments, virus particles comprising a payload, wherein the payload includes a nucleic acid that includes a heterologous transgene are provided. In some embodiments, the heterologous transgene encodes an RNA interference agent, for example a siRNA, shRNA or other interfering nucleic acid.

In some embodiments, the payload includes a heterologous transgene that encodes a therapeutic polypeptide. In some aspects, the heterologous transgene is a human gene or fragment thereof. In some aspects, the therapeutic polypeptide is a human protein. In some embodiments, the heterologous transgene of the virus particle encodes a molecule useful in treating a disease, and the virus particle is administered to a patient in need thereof to treat said disease. In some aspects the payload comprises a molecule that is effective in treating chronic CNS disease, such as, for example, an RNA interference nucleotide (e.g., shRNA, siRNA or miRNA that inhibits APOL-1). Examples of diseases (and heterologous transgenes or molecules encoded by said heterologous transgenes) according to the present disclosure include: MPSI (alpha-L-iduronidase (IDUA)); MPS II—Hunter syndrome (iduronate-2-sulfatase (IDS)); Ceroid lipofuscinosis-Batten disease (CLN1, CLN2, CLN10, CLN13, CLN5, CLN11, CLN4, CNL14, CLN3, CLN6, CLN7, CLN8, CLN12); MPS IIIa-Sanfilippo Type A syndrome (heparin sulfate sulfatase (also called N-sulfoglucosamine sulfohydrolase (SGSH)); MPS IIIB—Sanfilippo Type b syndrome (N-acetyl-alpha-D-glucosaminidase (NAGLU)); MPS VI—Maroteaux-Lamy syndrome (arylsulfatase B); MPS IV A—Morquio syndrome type A (GALNS); MPS IV B—Morquio syndrome type B (GLB1); chronic or neuropathic pain; Osteogenesis Imperfecgta Type I, II, III or IV (COL1A1 and/or COL1A2); hereditary angioedema (SERPING1, C1NH); Osteogenesis Imperfecta Type V (IFITM5); Osteogenesis Imperfecta Type VI (SERPINF1); Osteogenesis Imperfecta Type VII (CRTAP); Osteogenesis Imperfecta Type VIII (LEPRE1 and/or P3H1); Osteogenesis Imperfecta Type IX (PPIB); Gaucher disease type I, II and Ill (Glucocerebrosidase; GBAI); Parkinson's Disease (Glucocerebrosidase; GBAI and/or dopamine decarboxylase); Pompe (acid maltase; GAA; hGAA); Metachromatic leukodystrophy (Aryl sulfatase A); MPS VII-Sly syndrome (beta-glucuronidase); MPS VIII (glucosamine-6-sulfate sulfatase); MPS IX (Hyaluronidase); maple syrup urine disease (BCKDHA, BCKDHB, and/or DBT); Niemann-Pick disease (Sphingomyelinase); Parkinson's disease (anti-alpha synuclein RNAi); Alzheimer's disease (anti-mutant APP RNAi); Niemann-Pick disease without sphingomyelinase deficiency (NPC1 or NPC gene encoding a cholesterol metabolizing enzyme); Tay-Sachs disease (alpha subunit of beta-hexosaminidase); Sandhoff disease (both alpha and beta subunit of beta-hexosaminidase); Fabry Disease (alpha-galactosidase); Fucosidosis (fucosidase (FUCAI)); Alpha-mannosidosis (alpha-mannosidase); Beta-mannosidosis (beta-mannosidase); Wolman disease (cholesterol ester hydrolase); Dravet syndrome (SCN1A, SCN1B, SCN2A, GABRG2); Parkinson's disease (Neurturin); Parkinson's disease (glial derived growth factor (GDGF)); Parkinson's disease (tyrosine hydroxylase); frontotemporal dementia (progranulin); Angleman syndrome (ubiquitin protein ligase 3A (UBE3A), gene editing systems targeting a UBE3A inhibitory RNA (UBE3A-anitsense transcript)); Parkinson's disease (glutamic acid decarboxylase; FGF-2; BDGF); Spinal Muscular Atrophy (SMN, including SMN1 or SMN2); Friedreich's ataxia (Frataxin); Amyotrophic lateral sclerosis (ALS) (SOD1 inhibitor, e.g., anti-SOD1 RNAi); Glycogen Storage Disease Ia (Glucose-6-phosphatase); XLM™ (MTMI); Crigler Najjar (UGTIAI); CPVT (CASQ2); spinocerebellar ataxia (ATXN2; ATXN3 or other ATXN gene; anti-mutant Machado-Joseph disease/SCA3 allele RNAi); Rett syndrome (MECP2 or fragment thereof); Achromatopsia (CNGB3, CNGA3, GNAT2, PDE6C); Choroidermia (CDM); Danon Disease (LAMP2); Cystic Fibrosis (CFTR or fragment thereof); Duchenne Muscular Dystrophy (Mini-/Micro-Dystrophin Gene); SARS-Cov-2 infection (anti-SARS-Cov-2 RNAi, SARS-Cov-2 genome fragments or S protein (including variants)); Limb Girdle Muscular Dystrophy Type 2C—Gamma-sarcoglycanopathy (human-alpha-sarcoglycan); Advanced Heart Failure (SERCA2a); Rheumatoid Arthritis (TNFR:Fc Fusion; anti-TNF antibody or fragment thereof); Leber Congenital Amaurosis (GAA); X-linked adrenoleukodystrophy (ABCD1); Limb Girdle Muscular Dystrophy Type 2C-Gamma-sarcoglycanopathy (gamma-sarcoglycan); Angelman syndrome (UBE3A); Retinitis Pigmentosa (hMERTK); Age-Related Macular Degeneration (sFLT01); Phelan-McDermid syndrome (SHANK3; 22q13.3 replacement); Becker Muscular Dystrophy and Sporadic Inclusion Body Myositis (huFollistatin344); Parkinson's Disease (GDNF); Metachromatic Leukodystrophy—MLD (cuARSA); Hepatitis C (anti-HCV RNAi); Limb Girdle Muscular Dystrophy Type 2D (hSGCA); Human Immunodeficiency Virus Infections; (PG9DP); Acute Intermittant Porphyria (PBGD); Leber's Hereditary Optical Neuropathy (PIND4v2); Alpha-1 Antitrypsin Deficiency (alphalAT); X-linked Retinoschisis (RS1); Choroideremia (hCHM); Giant Axonal Neuropathy (GAN); Hemophilia B (Factor IX); Homozygous FH (hLDLR); Dysferlinopathies (DYSF); Achromatopsia (CNGA3 or CNGB3); Progressive supranuclear palsy (MAPT; anti-Tau; anti-MAPT RNAi); Omithine Transcarbamylase deficiency (OTC); Hemophilia A (Factor VIII); Age-related macular degeneration (AMD), including wetAMD (anti-VEGF antibody or RNAi); X-Linked Retinitis Pigmentosa (RPGR); Myotonic dystrophy Type 1 (DMPK; anti-DMPK RNAi, including anti-CTG trinucleotide repeat RNAi); Myotonic dystrophy Type 2 (CNBP); Facioscapulohumeral muscular dystrophy (D4Z4 DNA); oculopharynggeal muscular dystrophy (PABPN1; mutated PABPN1 inhibitor (e.g., RNAi)); Mucopolysaccharidosis Type VI (hARSB); Leber Hereditary Optic Neuropathy (ND4); X-Linked myotubular Myopathy (MTMI); Crigler-Najjar Syndrome (UGTIAI); Retinitis Pigmentosa (hPDE6B); Mucopolysaccharidosis Type 3B (hNAGLU); Duchenne Muscular Dystrophy (GALGT2); Alzheimer's Disease (NGF; ApoE4; ApoE2; ApoE3; Anti-ApoE RNAi, MAPT, anti-Tau antibody, anti-amyloid beta antibody (e.g., aducanumab)); multiple system atrophy; Familial Lipoprotein Lipase Deficiency (LPL); Alpha-1 Antitrypsin Deficiency (hAAT); Leber Congenital Amaurosis 2 (hRPE65v2); Batten Disease; Late Infantile Neuronal Lipofuscinosis (CLN2); Huntington's disease (HTT; anti-HTT RNAi); Fragile X syndrome (FMR1); Fragile X-associated tremor/ataxia syndrome (FMR1), Premature ovarian aging (FMR1), Polycystic ovarian syndrome (FMR1), Leber's Hereditary Optical Neuropathy (PIND4v2); Aromatic Amino Acid Decarboxylase Deficiency (hAADC); Retinitis Pigmentosa (hMERKTK); and Retinitis Pigmentosa (RLBPI). In some embodiments, the CNS disease is a tauopathy (e.g., Alzheimers' disease, progressive supranuclear palsy, frontotemporal dementia (Pick disease), corticobasal degeneration, argyrophilic grain disease, globular glial tauopathies, neurofibrillary tangle dementia, chronic traumatic encephalopathy (CTE), or aging-related tau astrogliopathy) and the payload is an anti-Tau antibody or antisense oligonucleotide targeting MAPT. In some aspects, the payload comprises a molecule that is effective in treating muscle-related disease. Exemplary, non-limiting muscle-related diseases are described below.

In some embodiments, the heterologous transgene encodes a therapeutic polypeptide. In some aspects, the heterologous transgene is a human gene or fragment thereof. In some aspects, the therapeutic polypeptide is a human protein. In some aspects, the heterologous transgene encodes an antibody or fragment thereof (for example an antibody light chain, an antibody heavy chain, a Fab or an scFv). Examples of antibodies or fragments thereof that are encoded by the heterologous transgene include but are not limited to; and an anti-Ab antibody (e.g., solanezumab, GSK933776, and lecanemab), anti-sortilin (e.g., AL-001), anti-Tau (e.g., ABBV-8E12, UCB-0107, and NI-105), anti-SEMA4D (e.g., VX15/2503), anti-alpha synuclein (e.g., prasinezumab, NI-202, and MED-1341), anti-SOD1 (e.g., NI-204), anti-CGRP receptor (e.g., eptinezumab, fremanezumab, or galcanezumab), anti-VEGF (e.g., sevacizumab, ranibizumab, bevacizumab, and brolucizumab), anti-EpoR (e.g., LKA-651,), anti-ALKI (e.g., ascrinvacumab), anti-C5 (e.g., tesidolumab, ravulizumab, and eculizumab), anti-CD105 (e.g., carotuximab), anti-CCIQ (e.g., ANX-007), anti-TNFa (e.g., adalimumab, infliximab, and golimumab), anti-RGMa (e.g., elezanumab), anti-TTR (e.g., NI-301 and PRX-004), anti-CTGF (e.g., pamrevlumab), anti-IL6R (e.g., satralizumab, tocilizumab, and sarilumab), anti-IL6 (e.g., siltuximab, clazakizumab, sirukumab, olokizumab, and gerilimzumab), anti-IL4R (e.g., dupilumab), anti-IL17A (e.g., ixekizumab and secukinumab), anti-ILSR (e.g., reslizumab), anti-IL-5 (e.g., benralizumab and mepolizumab), anti-IL13 (e.g., tralokinumab), anti-IL12/IL23 (e.g., ustekinumab), anti-CD 19 (e.g., inebilizumab), anti-IL31RA (e.g., nemolizumab), anti-ITGF7 mAb (e.g., etrolizumab), anti-SOST mAb (e.g., romosozumab), anti-IgE (e.g., omalizumab), anti-TSLP (e.g., nemolizumab), anti-pKal mAb (e.g., lanadelumab), anti-ITGA4 (e.g., natalizumab), anti-ITGA4B7 (e.g., vedolizumab), anti-BLyS (e.g., belimumab), anti-PD-1 (e.g., nivolumab and pembrolizumab), anti-RANKL (e.g., denosumab), anti-PCSK9 (e.g., alirocumab and evolocumab), anti-ANGPTL3 (e.g., evinacumab*), anti-OxPL (e.g., E06), anti-fD (e.g., lampalizumab), or anti-MMP9 (e.g., andecaliximab), optionally wherein the heavy chain (Fab and Fc region) and the light chain are separated by a self-cleaving furin (F)/F2A or furin (F)/T2A, IRES site, or flexible linker, for example, ensuring expression of equal amounts of the heavy and the light chain polypeptides.

In some embodiments, the virus particle comprises a heterologous transgene encoding a genome editing system. Examples include a CRISPR genome editing system (e.g., one or more components of a CRISPR genome editing system such as, for example, a guide RNA molecule and/or a RNA-guided nuclease such as a Cas enzyme such as Cas9, Cpf1 and the like), a zinc finger nuclease genome editing system, a TALEN genome editing system or a meganuclease genome editing system. In some embodiments, the genome editing system targets a mammalian, e.g., human, genomic target sequence. In some embodiments, the virus particle includes a heterologous transgene encoding a targetable transcription regulator. Examples include a CRISPR-based transcription regulator (for example, one or more components of a CRISPR-based transcription regulator, for example, a guide RNA molecule and/or a enzymatically-inactive RNA-guided nuclease/transcription factor (“TF”) fusion protein such as a dCas9-TF fusion, dCpf1-TF fusion and the like), a zinc finger transcription factor fusion protein, a TALEN transcription regulator or a meganuclease transcription regulator.

In some embodiments, components of a therapeutic molecule or system are delivered by more than one unique virus particle (e.g., a population that includes more than one unique virus particles). In other embodiments, the therapeutic molecule or components of a therapeutic molecule or system are delivered by a single unique virus particle (e.g., a population that includes a single unique virus particle).

In some embodiments, the transgene encodes any biologically active product or other product, e.g., a product desirable for study. Suitable transgenes may be readily selected by persons of skill in the art, such as those, but not limited to, those described herein.

Other examples of proteins encoded for by the transgene include, but are not limited to, colony stimulating factors (CSF); blood factors, such as β-globin, hemoglobin, tissue plasminogen activator or an analog thereof such as reteplase, lanoteplase or tenecteplase, and coagulation factors; interleukins; soluble receptors, such as soluble TNF-α receptors, soluble VEGF receptors, soluble interleukin receptors (e.g., soluble IL-1 receptors and soluble type II IL-1 receptors), or ligand-binding fragments of a soluble receptor; growth factors, such as keratinocyte growth factor (KGF), stem cell factor (SCF), or fibroblast growth factor (FGF, such as basic FGF and acidic FGF); enzymes; chemokines; enzyme activators, such as tissue plasminogen activator; angiogenic agents, such as vascular endothelial growth factors, glioma-derived growth factor, angiogenin, or angiogenin-2; anti-angiogenic agents, such as a soluble VEGF receptor; a protein vaccine; neuroactive peptides, such as nerve growth factor (NGF) or oxytocin; thrombolytic agents; tissue factors; macrophage activating factors; tissue inhibitors of metalloproteinases; or IL-1 receptor antagonists.

In some embodiments, the disclosure provides a nucleotide sequence which encodes a molecule for the treatment of Alzheimer's disease. In some embodiments, the molecule for the treatment of Alzheimer's disease comprises an inhibitory nucleic acid molecule (e.g., an antisense oligonucleotide or inhibitory RNA (e.g., siRNA, miRNA or shRNA molecule). In some embodiments, the molecule for the treatment of Alzheimer's disease comprises an inhibitory nucleic acid molecule (e.g., an antisense oligonucleotide or inhibitory RNA (e.g., siRNA, miRNA or shRNA molecule) targeting beta-amyloid, alpha-synuclein, Tau, TREM, e.g., TREM2, or an apolipoprotein (APO) E protein, e.g. APOE1, APOE2, APOE3 or APOE4.

In some embodiments, the molecule for the treatment of Alzheimer's disease comprises a genome editing system (for example a zinc finger nuclease, a meganuclease, a TALEN, or an RNA-guided genome editing system (e.g. a Cas polypeptide and a guide RNA molecule). In some embodiments the genome editing system targets a genetic region encoding a beta-amyloid protein or a Tau protein. In some embodiments, the genome editing system targets MSA4.

In some embodiments, the molecule for the treatment of Alzheimer's disease is an antibody or antigen-binding fragment thereof (e.g., as scFV). In some embodiments, the molecule for the treatment of Alzheimer's disease is a human protein or fragment or variant thereof. In some embodiments, the molecule for the treatment of Alzheimer's disease is an inhibitor of beta-amyloid aggregation. In some embodiments, the molecule for the treatment of Alzheimer's disease is an inhibitor of alpha-synuclein. In some embodiments, the molecule for the treatment of Alzheimer's disease is an anti-beta amyloid antibody, e.g., gantenerumab, crenezumab, aducanumab, lecanemab, bapineuzumab, solanezumab, donanemab or trontinemab (which is an anti-beta amyloid/anti-transferrin receptor bispecific antibody). In some embodiments, the molecule for the treatment of Alzheimer's disease is a Tau inhibitor, e.g., an anti-tau antibody (e.g., semorinemab). In some embodiments, the molecule for the treatment of Alzheimer's disease is an anti-TREM antibody or antigen-binding fragment thereof. In some embodiments, the molecule for the treatment of Alzheimer's disease is human nerve growth factor or a fragment or variant thereof. In some embodiments, the molecule for the treatment of Alzheimer's disease is human brain-derived neurotrophic factor or a fragment or variant thereof. In some embodiments, the molecule for the treatment of Alzheimer's disease is human synapsin-caveolin-1 (SynCav1) or a fragment or variant thereof.

Accordingly, in certain aspects, is the disclosure provides a virus particle comprising (a) a variant capsid polypeptide described herein, e.g., a capsid polypeptide described in Section 6.2 and (b) an engineered viral genome comprising (i) a nucleotide sequence encoding a molecule for the treatment of Alzheimer's disease, for example as described herein, and (ii) a promoter operably linked to said nucleotide sequence. The viral genome can further comprise one or more (e.g., two, three, four or all five) of (a) a pair of dependoparvovirus ITRs, (b) an intron, (c) an enhancer or repressor sequence, (d) a stuffer sequence, and (e) a polyA sequence. Preferably, the viral genome comprises ITRs flanking the nucleotide sequence and a polyA sequence operably linked to the nucleotide sequence. Typically, the viral genome lacks rep and cap sequences, which are in trans by the host cell in which the virus particle is produced. In some embodiments, the viral genome is self-complementary.

In certain aspects, the disclosure provides a virus particle comprising (a) a capsid polypeptide described in Section 6.2; (b) an engineered viral genome comprising (i) a nucleotide sequence encoding a molecule for the treatment of Alzheimer's disease, for example as described herein, and (ii) a promoter operably linked to said nucleotide sequence.

In certain aspects, the disclosure provides a virus particle comprising (a) a capsid polypeptide described herein, for example, a capsid polypeptide described in Section 6.2 and (b) an engineered viral genome comprising (i) a nucleotide sequence encoding a molecule for the treatment of Alzheimer's disease, for example gantenerumab or an antigen binding fragment thereof, and (ii) a promoter operably linked to said nucleotide sequence. The viral genome can further comprise one or more (e.g., two, three, four, or all five) of (a) a pair of dependoparvovirus ITRs, (b) an intron, (c) an enhancer or repressor sequence, (d) a stuffer sequence, and (e) a polyA sequence. Preferably, the viral genome comprises ITRs flanking the nucleotide sequence and a polyA sequence operably linked to the nucleotide sequence. Typically, the viral genome lacks rep and cap sequences, which are in trans by the host cell in which the virus particle is produced. In some embodiments, the viral genome is self-complementary.

In some embodiments, described herein is a virus particle comprising (a) a capsid polypeptide described herein, for example, a capsid polypeptide described in Section 6.2 and (b) an engineered viral genome comprising (i) a nucleotide sequence encoding a molecule for the treatment of Alzheimer's disease, for example crenezumab or an antigen binding fragment thereof, and (ii) a promoter operably linked to said nucleotide sequence. The viral genome can further comprise one or more (e.g., two, three, four, or all five) of (a) a pair of dependoparvovirus ITRs, (b) an intron, (c) an enhancer or repressor sequence, (d) a stuffer sequence, and (e) a polyA sequence. Preferably, the viral genome comprises ITRs flanking the nucleotide sequence and a polyA sequence operably linked to the nucleotide sequence. Typically, the viral genome lacks rep and cap sequences, which are in trans by the host cell in which the virus particle is produced. In some embodiments, the viral genome is self-complementary.

In certain aspects, the disclosure provides a virus particle comprising (a) a capsid polypeptide described herein, for example, a capsid polypeptide described in Section 6.2 and (b) an engineered viral genome comprising (i) a nucleotide sequence encoding a molecule for the treatment of myasthenia Gravis disease, for example an anti-IL-6 antibody (e.g., satralizumab) or antigen-binding fragment thereof, and (ii) a promoter operably linked to said nucleotide sequence. The viral genome can further comprise one or more (e.g., two, three, four, or all five) of (a) a pair of dependoparvovirus ITRs, (b) an intron, (c) an enhancer or repressor sequence, (d) a stuffer sequence, and (e) a polyA sequence.

Preferably, the viral genome comprises ITRs flanking the nucleotide sequence and a polyA sequence operably linked to the nucleotide sequence. Typically, the viral genome lacks rep and cap sequences, which are in trans by the host cell in which the virus particle is produced. In some embodiments, the viral genome is self-complementary.

In some embodiments, the disclosure provides a nucleotide sequence which encodes a molecule for the treatment of Parkinson's disease. In some embodiments, the molecule for the treatment of Parkinson's disease comprises an inhibitory nucleic acid molecule (e.g., an antisense oligonucleotide or inhibitory RNA (e.g., siRNA, miRNA or shRNA molecule). In some embodiments, the molecule for the treatment of Parkinson's disease comprises an inhibitory nucleic acid molecule (e.g., an antisense oligonucleotide or inhibitory RNA (e.g., siRNA, miRNA or shRNA molecule) targeting SNCA. An exemplary accession number for human SNCA is set forth in Table C1, together with example antisense oligonucleotide targeting SNCA.

In some embodiments, the molecule for the treatment of Parkinson's disease comprises a genome editing system (for example a zinc finger nuclease, a meganuclease, a TALEN, or an RNA-guided genome editing system (e.g. a Cas polypeptide and a guide RNA molecule). In some embodiments the genome editing system targets a genetic region encoding an alpha-synuclein protein (e.g., SNCA gene).

In certain aspects, the disclosure provides a virus particle comprising (a) a capsid polypeptide described herein, for example, a capsid polypeptide described in Section 6.2 and (b) an engineered viral genome comprising (i) a nucleotide sequence encoding a molecule for the treatment of Parkinson's disease, for example an anti-alpha synuclein antibody (e.g., prasinezumab or BIIB054) or antigen-binding fragment thereof, and (ii) a promoter operably linked to said nucleotide sequence. In some embodiments, described herein is a virus particle comprising (a) a capsid polypeptide described herein, for example, a capsid polypeptide described in Section 6.2 and (b) an engineered viral genome comprising (i) a nucleotide sequence (e.g., a human GBA1 gene or fragment or variant thereof, e.g., a variant thereof having at least 90% or at least 95% sequence identity to human GBA1) encoding a molecule for the treatment of Parkinson's disease, for example human beta-glucocerebrosidase or a fragment thereof, and (ii) a promoter operably linked to said nucleotide sequence. An exemplary accession number for human GBA1 is set forth in Table C1. Example human GBA1 variants (e.g., single nucleotide polymorphism containing variants) are disclosed in PCT Patent Application Publication No. WO/2023/004370/A1, incorporated herein by reference in its entirety. In some embodiments, described herein is a virus particle comprising (a) a capsid polypeptide described herein, for example, a capsid polypeptide described in Section 6.2 and (b) an engineered viral genome comprising (i) a nucleotide sequence encoding a molecule for the treatment of Parkinson's disease, for example an inhibitor of LRRK2, and (ii) a promoter operably linked to said nucleotide sequence. In some embodiments, described herein is a virus particle comprising (a) a capsid polypeptide described herein, for example, a capsid polypeptide described in Section 6.2 and (b) an engineered viral genome comprising (i) a nucleotide sequence encoding a molecule for the treatment of Parkinson's disease, for example a trophic factor (e.g., glial cell line-derived neurotrophic factor (GDNF) or cerebral dopamine neurotrophic factor (CDNF)) or a fragment thereof, and (ii) a promoter operably linked to said nucleotide sequence. In some embodiments, described herein is a virus particle comprising (a) a capsid polypeptide described herein, for example, a capsid polypeptide described in Section 6.2 and (b) an engineered viral genome comprising (i) a nucleotide sequence encoding a molecule for the treatment of Parkinson's disease, for example an antisense RNA (e.g., antisense RNA targeting alpha-synuclein, or SNCA, gene) or a fragment thereof, and (ii) a promoter operably linked to said nucleotide sequence. The viral genome can further comprise one or more (e.g., two, three, four, or all five) of (a) a pair of dependoparvovirus ITRs, (b) an intron, (c) an enhancer or repressor sequence, (d) a stuffer sequence, and (e) a polyA sequence. Preferably, the viral genome comprises ITRs flanking the nucleotide sequence and a polyA sequence operably linked to the nucleotide sequence. Typically, the viral genome lacks rep and cap sequences, which are in trans by the host cell in which the virus particle is produced. In some embodiments, the viral genome is self-complementary.

In certain aspects, the disclosure provides a virus particle comprising (a) a capsid polypeptide described herein, for example, a capsid polypeptide described in Section 6.2 and (b) an engineered viral genome comprising (i) a nucleotide sequence (e.g., a human GBA gene, e.g., a human GBA1 gene) encoding a molecule for the treatment of Gaucher's disease, for example a human glucocerebrosidase (GCase, e.g. beta-glucosylceramidase-1) or fragment or variant thereof, and (ii) a promoter operably linked to said nucleotide sequence. The viral genome can further comprise one or more (e.g., two, three, four, or all five) of (a) a pair of dependoparvovirus ITRs, (b) an intron, (c) an enhancer or repressor sequence, (d) a stuffer sequence, and (e) a polyA sequence. Preferably, the viral genome comprises ITRs flanking the nucleotide sequence and a polyA sequence operably linked to the nucleotide sequence. Typically, the viral genome lacks rep and cap sequences, which are in trans by the host cell in which the virus particle is produced. In some embodiments, the viral genome is self-complementary.

In certain aspects, the disclosure provides a virus particle comprising (a) a capsid polypeptide described herein, for example, a capsid polypeptide described in Section 6.2 and (b) an engineered viral genome comprising (i) a nucleotide sequence encoding a molecule for the treatment of multiple sclerosis, for example an anti-CD20 antibody (e.g., ocrelizumab, rituximab, ofatumumab, or RG6035/R07121932 (an anti-CD20, anti-transferrin receptor bispecific antibody sometimes known as Brainshuttle (BS) CD20-Multiple Sclerosis) or antigen-binding fragment thereof, and (ii) a promoter operably linked to said nucleotide sequence. In some embodiments the multiple sclerosis is relapsing remitting multiple sclerosis. In some embodiments the multiple sclerosis is primary progressing multiple sclerosis. In some embodiments the multiple sclerosis is secondary progressive multiple sclerosis. The viral genome can further comprise one or more (e.g., two, three, four, or all five) of (a) a pair of dependoparvovirus ITRs, (b) an intron, (c) an enhancer or repressor sequence, (d) a stuffer sequence, and (e) a polyA sequence. Preferably, the viral genome comprises ITRs flanking the nucleotide sequence and a polyA sequence operably linked to the nucleotide sequence. Typically, the viral genome lacks rep and cap sequences, which are in trans by the host cell in which the virus particle is produced. In some embodiments, the viral genome is self-complementary.

In certain aspects, the disclosure provides a virus particle comprising (a) a capsid polypeptide described herein, for example, a capsid polypeptide described in Section 6.2 and (b) an engineered viral genome comprising (i) a nucleotide sequence encoding a molecule for the treatment of Huntington's disease, for example an inhibitory nucleic acid directed to mutated huntingtin protein (HTT) (e.g., tominersen, WVE-120101 or WVE-120102), and (ii) a promoter operably linked to said nucleotide sequence. The viral genome can further comprise one or more (e.g., two, three, four, or all five) of (a) a pair of dependoparvovirus ITRs, (b) an intron, (c) an enhancer or repressor sequence, (d) a stuffer sequence, and (e) a polyA sequence. Preferably, the viral genome comprises ITRs flanking the nucleotide sequence and a polyA sequence operably linked to the nucleotide sequence. Typically, the viral genome lacks rep and cap sequences, which are in trans by the host cell in which the virus particle is produced. In some embodiments, the viral genome is self-complementary.

In certain aspects, the disclosure provides a virus particle comprising (a) a capsid polypeptide described herein, for example, a capsid polypeptide described in Section 6.2 and (b) an engineered viral genome comprising (i) a nucleotide sequence encoding a molecule for the treatment of Phelan McDermid syndrome, for example human SHANK3 or fragment or variant thereof (e.g., a variant thereof having at least 90% or at least 95% sequence identity to human SHANK3), and (ii) a promoter operably linked to said nucleotide sequence. In some embodiments, described herein is a virus particle comprising (a) a capsid polypeptide described herein, for example, a capsid polypeptide described in Section 6.2 and (b) an engineered viral genome comprising (i) a nucleotide sequence encoding a molecule for the treatment of Phelan McDermid syndrome, for example a human growth hormone, e.g., human insulin like growth factor 1 (IGF-1), or fragment or variant thereof (e.g., a variant thereof having at least 90% or at least 95% sequence identity to human IGF-1), and (ii) a promoter operably linked to said nucleotide sequence. The viral genome can further comprise one or more (e.g., two, three, four, or all five) of (a) a pair of dependoparvovirus ITRs, (b) an intron, (c) an enhancer or repressor sequence, (d) a stuffer sequence, and (e) a polyA sequence. Preferably, the viral genome comprises ITRs flanking the nucleotide sequence and a polyA sequence operably linked to the nucleotide sequence. Typically, the viral genome lacks rep and cap sequences, which are in trans by the host cell in which the virus particle is produced. In some embodiments, the viral genome is self-complementary.

In certain aspects, the disclosure provides a virus particle comprising (a) a capsid polypeptide described herein, for example, a capsid polypeptide described in Section 6.2 and (b) an engineered viral genome comprising (i) a nucleotide sequence encoding a molecule for the treatment of frontotemporal dementia, for example human progranulin or granulin, or fragment or variant thereof, and (ii) a promoter operably linked to said nucleotide sequence. In some embodiments, described herein is a virus particle comprising (a) a capsid polypeptide described herein, for example, a capsid polypeptide described in Section 6.2 and (b) an engineered viral genome comprising (i) a nucleotide sequence encoding a molecule for the treatment of frontotemporal dementia, for example an anti-tau antibody (e.g., semorinemab), or fragment or variant thereof, and (ii) a promoter operably linked to said nucleotide sequence. In some embodiments, described herein is a virus particle comprising (a) a capsid polypeptide described herein, for example, a capsid polypeptide described in Section 6.2 and (b) an engineered viral genome comprising (i) a nucleotide sequence encoding a molecule for the treatment of frontotemporal dementia, for example an inhibitory nucleic acid which targets SOD-1 (e.g., tofersen), and (ii) a promoter operably linked to said nucleotide sequence. The viral genome can further comprise one or more (e.g., two, three, four, or all five) of (a) a pair of dependoparvovirus ITRs, (b) an intron, (c) an enhancer or repressor sequence, (d) a stuffer sequence, and (e) a polyA sequence. Preferably, the viral genome comprises ITRs flanking the nucleotide sequence and a polyA sequence operably linked to the nucleotide sequence. Typically, the viral genome lacks rep and cap sequences, which are in trans by the host cell in which the virus particle is produced. In some embodiments, the viral genome is self-complementary.

In certain aspects, the disclosure provides a virus particle comprising (a) a capsid polypeptide described herein, for example, a capsid polypeptide described in Section 6.2 and (b) an engineered viral genome comprising (i) a nucleotide sequence encoding a molecule for the treatment of amyotrophic lateral sclerosis (ALS), for example an inhibitory nucleic acid which targets SOD-1 (e.g., tofersen), and (ii) a promoter operably linked to said nucleotide sequence. In some embodiments, described herein is a virus particle comprising (a) a capsid polypeptide described herein, for example, a capsid polypeptide described in Section 6.2 and (b) an engineered viral genome comprising (i) a nucleotide sequence encoding a molecule for the treatment of ALS, for example an inhibitory nucleic acid which targets C9orf72 (e.g., BIIB078), and (ii) a promoter operably linked to said nucleotide sequence. In some embodiments, described herein is a virus particle comprising (a) a capsid polypeptide described herein, for example, a capsid polypeptide described in Section 6.2 and (b) an engineered viral genome comprising (i) a nucleotide sequence encoding a molecule for the treatment of ALS, for example an inhibitory nucleic acid which targets ATXN2 (e.g., BIIB105), and (ii) a promoter operably linked to said nucleotide sequence. In some embodiments, described herein is a virus particle comprising (a) a capsid polypeptide described herein, for example, a capsid polypeptide described in Section 6.2 and (b) an engineered viral genome comprising (i) a nucleotide sequence encoding a molecule for the treatment of ALS, for example an inhibitory nucleic acid which targets FUS, and (ii) a promoter operably linked to said nucleotide sequence. The viral genome can further comprise one or more (e.g., two, three, four, or all five) of (a) a pair of dependoparvovirus ITRs, (b) an intron, (c) an enhancer or repressor sequence, (d) a stuffer sequence, and (e) a polyA sequence. Preferably, the viral genome comprises ITRs flanking the nucleotide sequence and a polyA sequence operably linked to the nucleotide sequence. Typically, the viral genome lacks rep and cap sequences, which are in trans by the host cell in which the virus particle is produced. In some embodiments, the viral genome is self-complementary.

In certain aspects, the disclosure provides a virus particle comprising (a) a capsid polypeptide described herein, for example, a capsid polypeptide described in Section 6.2 and (b) an engineered viral genome comprising (i) a nucleotide sequence encoding a molecule for the treatment of multiple system atrophy, for example an anti-alpha synuclein antibody (e.g., prasinezumab), and (ii) a promoter operably linked to said nucleotide sequence. In some embodiments, described herein is a virus particle comprising (a) a capsid polypeptide described herein, for example, a capsid polypeptide described in Section 6.2 and (b) an engineered viral genome comprising (i) a nucleotide sequence encoding a molecule for the treatment of multiple system atrophy, for example an antisense oligonucleotide targeting human SNCA, and (ii) a promoter operably linked to said nucleotide sequence. In some embodiments, described herein is a virus particle comprising (a) a capsid polypeptide described herein, for example, a capsid polypeptide described in Section 6.2 and (b) an engineered viral genome comprising (i) a nucleotide sequence encoding a molecule for the treatment of multiple system atrophy, for example human glial cell-derived neurotrophic factor (GDNF) or fragment or variant thereof (e.g., a variant thereof having at least 90% or at least 95% sequence identity to human GDNF), and (ii) a promoter operably linked to said nucleotide sequence. The viral genome can further comprise one or more (e.g., two, three, four, or all five) of (a) a pair of dependoparvovirus ITRs, (b) an intron, (c) an enhancer or repressor sequence, (d) a stuffer sequence, and (e) a polyA sequence. Preferably, the viral genome comprises ITRs flanking the nucleotide sequence and a polyA sequence operably linked to the nucleotide sequence. Typically, the viral genome lacks rep and cap sequences, which are in trans by the host cell in which the virus particle is produced. In some embodiments, the viral genome is self-complementary.

In certain aspects, the disclosure provides a virus particle comprising (a) a capsid polypeptide described herein, for example, a capsid polypeptide described in Section 6.2 and (b) an engineered viral genome comprising (i) a nucleotide sequence encoding a molecule for the treatment of progressive supranuclear palsy (PSP), for example an anti-Tau antibody (e.g., semorinemab), and (ii) a promoter operably linked to said nucleotide sequence. In some embodiments, described herein is a virus particle comprising (a) a capsid polypeptide described herein, for example, a capsid polypeptide described in Section 6.2 and (b) an engineered viral genome comprising (i) a nucleotide sequence encoding a molecule for the treatment of progressive supranuclear palsy (PSP), for example an anti-alpha-synuclein antibody (e.g., prasinezumab), and (ii) a promoter operably linked to said nucleotide sequence. The viral genome can further comprise one or more (e.g., two, three, four, or all five) of (a) a pair of dependoparvovirus ITRs, (b) an intron, (c) an enhancer or repressor sequence, (d) a stuffer sequence, and (e) a polyA sequence. Preferably, the viral genome comprises ITRs flanking the nucleotide sequence and a polyA sequence operably linked to the nucleotide sequence. Typically, the viral genome lacks rep and cap sequences, which are in trans by the host cell in which the virus particle is produced. In some embodiments, the viral genome is self-complementary.

In certain aspects, the disclosure provides a virus particle comprising (a) a capsid polypeptide described herein, for example, a capsid polypeptide described in Section 6.2 and (b) an engineered viral genome comprising (i) a nucleotide sequence encoding a molecule for the treatment of Freidrich's ataxia, for example human frataxin (FRXN) or fragment or variant thereof (e.g., a variant thereof having at least 90% or at least 95% sequence identity to human FRXN), and (ii) a promoter operably linked to said nucleotide sequence. The viral genome can further comprise one or more (e.g., two, three, four, or all five) of (a) a pair of dependoparvovirus ITRs, (b) an intron, (c) an enhancer or repressor sequence, (d) a stuffer sequence, and (e) a polyA sequence. Preferably, the viral genome comprises ITRs flanking the nucleotide sequence and a polyA sequence operably linked to the nucleotide sequence. Typically, the viral genome lacks rep and cap sequences, which are in trans by the host cell in which the virus particle is produced. In some embodiments, the viral genome is self-complementary.

In certain aspects, the disclosure provides a nucleotide sequence which encodes a molecule for the treatment of Angelman syndrome. In some embodiments, the molecule for the treatment of Angelman syndrome comprises an inhibitory nucleic acid molecule (e.g., an antisense oligonucleotide or inhibitory RNA (e.g., siRNA, miRNA or shRNA molecule). In some embodiments, the molecule for the treatment of Angelman syndrome comprises an inhibitory nucleic acid molecule (e.g., an antisense oligonucleotide or inhibitory RNA (e.g., siRNA, miRNA or shRNA molecule) targeting UBE3A.

In some embodiments, the molecule for the treatment of Angelman syndrome comprises a genome editing system (for example a zinc finger nuclease, a meganuclease, a TALEN, or an RNA-guided genome editing system (e.g. a Cas polypeptide and a guide RNA molecule). In some embodiments the genome editing system targets UBE3A.

In certain aspects, the disclosure provides a virus particle comprising (a) a capsid polypeptide described herein, for example, a capsid polypeptide described in Section 6.2 and (b) an engineered viral genome comprising (i) a nucleotide sequence encoding a molecule for the treatment of Angelman syndrome, for example an inhibitor of a UBE3A antisense nucleic acid (e.g., rugonersen) or a human UBE3A or fragment or variant thereof (e.g., a variant thereof having at least 90% or at least 95% sequence identity to human UBE3A), and (ii) a promoter operably linked to said nucleotide sequence. An exemplary accession number for human UBE3A is set forth in Table C1. The viral genome can further comprise one or more (e.g., two, three, four, or all five) of (a) a pair of dependoparvovirus ITRs, (b) an intron, (c) an enhancer or repressor sequence, (d) a stuffer sequence, and (e) a polyA sequence. Preferably, the viral genome comprises ITRs flanking the nucleotide sequence and a polyA sequence operably linked to the nucleotide sequence. Typically, the viral genome lacks rep and cap sequences, which are in trans by the host cell in which the virus particle is produced. In some embodiments, the viral genome is self-complementary.

In certain aspects, the disclosure provides a virus particle comprising (a) a capsid polypeptide described herein, for example, a capsid polypeptide described in Section 6.2 and (b) an engineered viral genome comprising (i) a nucleotide sequence encoding a molecule for the treatment of Fragile X syndrome, for example human fragile X mental retardation protein (FMRP) or fragment or variant thereof (e.g., a nucleotide sequence comprising an FMR1 gene or fragment or variant thereof), and (ii) a promoter operably linked to said nucleotide sequence. An exemplary accession number for human FMRP is set forth in Table C1. In some embodiments, described herein is a virus particle comprising (a) a capsid polypeptide described herein, for example, a capsid polypeptide described in Section 6.2 and (b) an engineered viral genome comprising (i) a nucleotide sequence encoding a molecule for the treatment of Fragile X syndrome, for example an inhibitor of transcriptional silencing of FMRP, and (ii) a promoter operably linked to said nucleotide sequence. In some embodiments, described herein is a virus particle comprising (a) a capsid polypeptide described herein, for example, a capsid polypeptide described in Section 6.2 and (b) an engineered viral genome comprising (i) a nucleotide sequence encoding a molecule for the treatment of Fragile X syndrome, for example human diacylglycerol kinase (DGKk) or fragment or variant thereof (e.g., a variant thereof having at least 90% or at least 95% sequence identity to human DGKk), and (ii) a promoter operably linked to said nucleotide sequence. An exemplary accession number for human DGKk is set forth in Table C1. The viral genome can further comprise one or more (e.g., two, three, four, or all five) of (a) a pair of dependoparvovirus ITRs, (b) an intron, (c) an enhancer or repressor sequence, (d) a stuffer sequence, and (e) a polyA sequence. Preferably, the viral genome comprises ITRs flanking the nucleotide sequence and a polyA sequence operably linked to the nucleotide sequence. Typically, the viral genome lacks rep and cap sequences, which are in trans by the host cell in which the virus particle is produced. In some embodiments, the viral genome is self-complementary.

In certain aspects, the disclosure provides a virus particle comprising (a) a capsid polypeptide described herein, for example, a capsid polypeptide described in Section 6.2 and (b) an engineered viral genome comprising (i) a nucleotide sequence encoding a molecule for the treatment of Rett syndrome, for example a human MECP2 or fragment or variant thereof, e.g., a variant thereof having at least 90% or at least 95% sequence identity to human MECP2, and (ii) a promoter operably linked to said nucleotide sequence. An exemplary accession number for human MECP2 is set forth in Table C1. The viral genome can further comprise one or more (e.g., two, three, four or all five) of (a) a pair of dependoparvovirus ITRs, (b) an intron, (c) an enhancer or repressor sequence, (d) a stuffer sequence, and (e) a polyA sequence. Preferably, the viral genome comprises ITRs flanking the nucleotide sequence and a polyA sequence operably linked to the nucleotide sequence. Typically, the viral genome lacks rep and cap sequences, which are in trans by the host cell in which the virus particle is produced. In some embodiments, the viral genome is self-complementary.

In certain aspects, the disclosure provides a virus particle comprising (a) a capsid polypeptide described herein, for example, a capsid polypeptide described in Section 6.2 and (b) an engineered viral genome comprising (i) a nucleotide sequence encoding a molecule for the treatment of Dravet syndrome, for example human sodium channel, voltage gated, type 1-alpha (SCN1A or Nav1.1) or fragment or variant thereof, and (ii) a promoter operably linked to said nucleotide sequence. In some embodiments, described herein is a virus particle comprising (a) a capsid polypeptide described herein, for example, a capsid polypeptide described in Section 6.2 and (b) an engineered viral genome comprising (i) a nucleotide sequence encoding a molecule for the treatment of Dravet syndrome, for example an inhibitory nucleic acid targeting a mutant SCN1A transcript, and (ii) a promoter operably linked to said nucleotide sequence. In some embodiments, described herein is a virus particle comprising (a) a capsid polypeptide described herein, for example, a capsid polypeptide described in Section 6.2 and (b) an engineered viral genome comprising (i) a nucleotide sequence encoding a molecule for the treatment of Dravet syndrome, for example an anti-tau antibody (e.g., semorinemab), or fragment or variant thereof, and (ii) a promoter operably linked to said nucleotide sequence. In some embodiments, described herein is a virus particle comprising (a) a capsid polypeptide described herein, for example, a capsid polypeptide described in Section 6.2 and (b) an engineered viral genome comprising (i) a nucleotide sequence encoding a molecule for the treatment of Dravet syndrome, for example human Syntaxin-binding protein 1 (STXBP1) or fragment or variant thereof, and (ii) a promoter operably linked to said nucleotide sequence. The viral genome can further comprise one or more (e.g., two, three, four, or all five) of (a) a pair of dependoparvovirus ITRs, (b) an intron, (c) an enhancer or repressor sequence, (d) a stuffer sequence, and (e) a polyA sequence. Preferably, the viral genome comprises ITRs flanking the nucleotide sequence and a polyA sequence operably linked to the nucleotide sequence. Typically, the viral genome lacks rep and cap sequences, which are in trans by the host cell in which the virus particle is produced. In some embodiments, the viral genome is self-complementary.

In certain aspects, the disclosure provides a virus particle comprising (a) a capsid polypeptide described herein, for example, a capsid polypeptide described in Section 6.2 and (b) an engineered viral genome comprising (i) a nucleotide sequence encoding a molecule for the treatment of Friedreich's ataxia, for example a human FXN gene or fragment or variant thereof, and (ii) a promoter operably linked to said nucleotide sequence. The viral genome can further comprise one or more (e.g., two, three, four, or all five) of (a) a pair of dependoparvovirus ITRs, (b) an intron, (c) an enhancer or repressor sequence, (d) a stuffer sequence, and (e) a polyA sequence. Preferably, the viral genome comprises ITRs flanking the nucleotide sequence and a polyA sequence operably linked to the nucleotide sequence. Typically, the viral genome lacks rep and cap sequences, which are in trans by the host cell in which the virus particle is produced. In some embodiments, the viral genome is self-complementary.

Further exemplary diseases that can be treated and further exemplary heterologous transgenes that can be delivered via the viral particles of the disclosure are provided in Table C1.

TABLE C1
Exemplary indications and transgenes

| | | UniProt |
|---|---|---|
| | | Accession # |
| Indication | Transgene | (Human) |
| Achromatopsia - color blindness | Cyclic nucleotide-gated cation channel | Q16281 |
| | alpha-3 (CNGA3) | |
| | Cyclic nucleotide-gated cation channel | Q9NQW8 |
| | beta-3 (CNGB3) | |
| | Guanine nucleotide-binding protein G(t) | P19087 |
| | subunit alpha-2 (GNAT2) | |
| | Cone cGMP-specific 3′,5′-cyclic | P51160 |
| | phosphodiesterase subunit alpha | |
| | (PDE6C) | |
| Acute Intermittent Porphyria | Porphobilinogen deaminase (PBGD), | P08397 |
| | HMBS | |
| Adie syndrome - Adie's pupil | MPZ | P25189 |
| Age-Related Macular Degeneration | Vascular endothelial growth factor | P17948 |
| | receptor 1 (FLT1) | |
| | Vascular endothelial growth factor A | P15692 |
| | (VEGFA) | |
| Agenesis of the Corpus Callosum | SLC12A6 | Q9UHW9 |
| (ACCPN) | | |
| Aicardi-Goutieres syndrome | TREX1 | Q9NSU2 |
| Alexander disease | GFAP | P14136 |
| Alpers syndrome | POLG | P54098 |
| Alternating hemiplegia | ATP1A2 | P50993 |
| | ATP1A3 | P13637 |
| Alzheimer's disease | NGF | P01138 |
| | ApoE | P02649 |
| | Presenilin (PSEN1) | A0A024R6A3 |
| | Presenilin-2 (PSEN2) | P49810 |
| | Amyloid-beta precursor protein (APP) | P05067 |
| | ADAM10 | O14672 |
| | MAPT, Tau | P10636 |
| Amyotrophic lateral sclerosis (ALS) - | Superoxide dismutase-1 (SOD1) | P00441 |
| Lou Gehrig's disease | | |
| Amyotrophy, hereditary neuralgic | SEPT9 | Q9UHD8 |
| Angelman syndrome | Ubiquitin-protein ligase E3A (UBE3A) | Q05086 |
| Aromatic L-amino acid decarboxylase | DDC | P20711 |
| deficiency (AADCD) | | |
| Ataxia | APTX | Q7Z2E3 |
| | KCNA1 | Q09470 |
| | CACNA1A | O00555 |
| Ataxia Telangiectasia - Louis-Bar | Serine-protein kinase ATM (ATM) | Q13315 |
| syndrome | | |
| Attention deficit hyperactivity disorder | DRD4 | P21917 |
| (ADHD) | CDH2 | P19022 |
| Becker muscular dystrophy | Follistatin (FST) | P19883 |
| | DMD | P11532 |
| Benign essential blepharospasm | DRD5 | P21918 |
| Bradbury-Eggleston syndrome - pure | COQ2 | Q96H96 |
| autonomic failure | | |
| Bulbar palsy (BVVLS1) | SLC52A3 | Q9NQ40 |
| Canavan disease - aminoacylase 2 | ASPA | P45381 |
| deficiency | | |
| Carpal tunnel syndrome | TTR | P02766 |
| Cavernoma - Cavernous angioma - | KRIT1 | O00522 |
| Cavernous malformations | | |
| Cerebellar Hypoplasia (CHEGDD) | OXR1 | Q8N573 |
| Cerebellar ataxia (CAMRQ2) | WDR81 | Q562E7 |
| Cerebral Arteriopathy with SCI and | NOTCH3 | Q9UM47 |
| Leukoencephalopathy (CADASIL) | | |
| Cerebral gigantism - Sotos syndrome 1 | NSD1 | Q96L73 |
| Cerebro-oculo-facio-skeletal syndrome | ERCC6 | Q03468 |
| (COFS) | | |
| Ceroid lipofuscinosis - Batten disease | CLN1 (PPT1) | P50897 |
| | CLN2 (TPP1) | O14773 |
| | CLN3 (battenin) | Q13286 |
| | CLN4 | Q9H3Z4 |
| | CLN5 | O75503 |
| | CLN6 | Q9NWW5 |
| | CLN7 (MFSD8) | Q8NHS3 |
| | CLN8 | Q9UBY8 |
| | CLN10 (cathepsin D) | P07339 |
| | CLN11 (progranulin) | P28799 |
| | CLN12 (ATP13A2) | Q9NQ11 |
| | CLN13 (cathepsin F) | Q9UBX1 |
| | CLN14 (KCTD7) | Q96MP8 |
| Charcot-Marie-Tooth disease | PMP22 | Q01453 |
| | MPZ | P25189 |
| | DNM2 | P50570 |
| | MFN2 | O95140 |
| | KIF1B | O60333 |
| | SBF2 | Q86WG5 |
| | PNKP | Q96T60 |
| | GDAP1 | Q8TB36 |
| | LMNA | P02545 |
| | FGD4 | Q96M96 |
| | MTMR2 | Q13614 |
| Chorea | NKX2-1 | P43699 |
| Choreoacanthocytosis | VPS13A | Q96RL7 |
| Choroideremia | Rab escort protein (Rep1), CHM | P24386 |
| Chronic Inflammatory Demyelinating | PMP22 | Q01453 |
| Polyneuropathy (CIDP) | | |
| Cockayne syndrome B (CSB) | ERCC6 | Q03468 |
| Coffin-Lowry syndrome | RPS6KA3 | P51812 |
| Craniosynostosis | MSX2 | P35548 |
| | TWIST1 | Q15672 |
| | SKI | P12755 |
| | SMAD6 | O43541 |
| Creutzfeldt-Jakob disease | PRNP | P04156 |
| | HLA-DQB1 | P01920 |
| Crigler-Najjar Syndrome - | UDP-glucuronosyltransferase 1A1 | P22309 |
| hyperbilirubinemia | (UGT1A1) | |
| Cushing Syndrome | PRKACA | P17612 |
| Dentatorubral atrophy (DRPLA) | ATN1 | P54259 |
| Developmental and Epileptic | ARX | Q96QS3 |
| Encephalopathy | FGF12 | P61328 |
| | PIGP | P57054 |
| | GABRB3 | P28472 |
| | NECAP1 | Q8NC96 |
| Developmental Dyspraxia - speech- | FOXP2 | O15409 |
| language disorder 1 (SPCH1) | | |
| Dravet syndrome | Sodium channel protein type 1 subunit | P35498 |
| | alpha (SCN1A) | |
| | SCN1B | Q07699 |
| | SCN2A | Q99250 |
| | GABA receptor subunit gamma-2 | P18507 |
| | (GABRG2) | |
| Dysautonomia - -Day | ELP1 | O95163 |
| syndrome | | |
| Dystonias | GCH1 | P30793 |
| | TOR1A | O14656 |
| | SGCE | O43556 |
| | TUBB4A | P04350 |
| Encephalocele | COL18A1 | P39060 |
| Epilepsy disorders | GRIN2A | Q12879 |
| | CSTB | P04080 |
| | STARD7 | Q9NQZ5 |
| | DEPDC5 | O75140 |
| | PCDH19 | Q8TAB3 |
| Essential tremor | DRD3 | P35462 |
| | NOTCH2NLC | P0DPK4 |
| | FUS | P35637 |
| Fabry disease | alpha-galactosidase A (GLA) | P06280 |
| Farber disease - ceramidase | ASAH1 | Q13510 |
| deficiency | | |
| Fahr disease | SLC20A2 | Q08357 |
| Febrile Seizures | GABRG2 | P18507 |
| | ADGRV1 | Q8WXG9 |
| | CPA6 | P11509 |
| | SCN1A | P35498 |
| Fragile X syndrome | FMR1 (FMRP) | Q06787 |
| | Diacylglycerol kinase (DGKk) | Q5KSL6 |
| Friedreich's ataxia | Frataxin (FXN) | Q16595 |
| Frontotemporal dementia | Progranulin (GRN) | P28799 |
| | MAPT (tau) | P10636 |
| | PSEN1 | A0A024R6A3 |
| Fucosidosis | alpha-L-fucosidase (FUCA1) | P04066 |
| Fundus albipunctatus | RLBP1 | P12271 |
| Gaucher disease, types I, II and III | Glucocerebrosidase (GBA1) | P04062 |
| Generalized gangliosidoses (GM1, | GLB1 | P16278 |
| GM2, GM3) | | |
| Gerstmann-Straussler-Scheinker | PRNP | P04156 |
| disease | | |
| Giant axonal neuropathy | Gigaxonin (GAN) | Q9H2C0 |
| Glycogen storage disease II - Pompe | Acid maltase, lysosomal alpha- | P10253 |
| disease - Acid Maltase Deficiency | glucosidase (LYAG, GAA) | |
| Guillain-Barre syndrome | PMP22 | Q01453 |
| Hallervorden-Spatz disease - PKAN - | PANK2 | Q9BZ23 |
| NBIA1 | | |
| Hemiplegia Alterans | ATP1A2 | P50993 |
| | ATP1A3 | P13637 |
| Hereditary Neuropathies | WNK1 | Q9H4A3 |
| | MFN2 | O95140 |
| | HK1 | P19367 |
| | TFG | Q92734 |
| | SPTLC1 | O15269 |
| Heredopathia Atactica | PHYH | O14832 |
| Polyneuritiformis - Refsum disease | | |
| Holoprosencephalies | GLI2 | P10070 |
| | TGIF1 | Q15583 |
| | ZIC2 | O95409 |
| | PTCH1 | Q13635 |
| | SHH | Q15465 |
| Huntington's disease | HTT | P42858 |
| Hydrocephalus disorders | CCDC88C | Q9P219 |
| | WDR81 | Q562E7 |
| | TRIM71 | Q2Q1W2 |
| | MPDZ | O75970 |
| Incontinentia Pigmenti | IKBKG | Q9Y6K9 |
| Infantile Hypotonia | NALCN | Q8IZF0 |
| | TBCK | Q8TEA7 |
| | CCDC174 | Q6PII3 |
| | UNC80 | Q8N2C7 |
| Infantile Neuroaxonal Dystrophy | PLA2G6 | O60733 |
| Infantile Phytanic Acid Storage | PEX1 | O43933 |
| Disease (PBD1B) | | |
| Joubert Syndrome | INPP5E | Q9NRR6 |
| Kennedy Disease | Androgen receptor (AR) | P10275 |
| Klippel-Feil Syndrome | GDF6 | Q6KF10 |
| Krabbe disease - GALC deficiency | GALC | P54803 |
| Lambert-Eaton Myasthenic Syndrome | CACNA1A | O00555 |
| | CACNB2 | Q13936 |
| Landau-Kleffner Syndrome | GRIN2A | Q12879 |
| Late infantile neuronal lipofuscinosis | TPP1 | O14773 |
| (CLN2) | | |
| Lesch-Nyhan Syndrome | HPRT1 | P00492 |
| Leber congenital amaurosis - retinal | Retinal guanylyl cyclase 1 (GUCY2D) | Q02846 |
| blindness | Retinoid isomerohydrolase (RPE65) | Q16518 |
| | Centrosomal protein of 290 kDa | O15078 |
| | (CEP290) | |
| | Protein crumbs homolog 1 (CRB1) | P82279 |
| Leber's hereditary optical neuropathy | NADH-ubiquinone oxidoreductase chain | P03905 |
| | 4 (ND4) | |
| Leukodystrophy | ARSA | P15289 |
| Levine-Critchley Syndrome - | VPS13A | Q96RL7 |
| choreoacanthocytosis | | |
| Lewy body dementia | SNCA | P37840 |
| | SNCB | Q16143 |
| Lipoid Proteinosis - Urbach-Wiethe | ECM1 | Q16610 |
| disease | | |
| Lissencephaly | PAFAH1B1 | Q9PTR5 |
| | NDE1 | Q9NXR1 |
| | TUBA1A | Q71U36 |
| | LAMB1 | LAMB1 |
| | KATNB1 | Q9BVA0 |
| | RELN | P78509 |
| Macrocephaly/ Megalencephaly | TBC1D7 | Q9P0N9 |
| Menkes Disease | ATP7A | Q04656 |
| Metachromatic Leukodystrophy - MLD | Arylsulfatase A (ARSA) | P15289 |
| Microcephaly diseases | KIF11 | P52732 |
| | MCPH1 | Q8NEM0 |
| | SLC25A19 | Q9HC21 |
| Migraine, familial hemiplegic | CACNA1A | O00555 |
| | ATP1A2 | P50993 |
| | SCN1A | P35498 |
| Mitochondrial DNA depletion | RRM2B | Q7LG56 |
| syndromes | DGUOK | Q16854 |
| | POLG | P54098 |
| | TYMP | P19971 |
| | TK2 | O00142 |
| Morvan disease | WNK1 | Q9H4A3 |
| Mucolipidosis | GNPTAB | Q3T906 |
| | MCOLN1 | Q9GZU1 |
| Mucopolysaccharidosis Type I (MPS I) - | alpha-L-iduronidase (IDUA) | P35475 |
| Hurler syndrome | | |
| MPS II - Hunter syndrome | iduronate-2-sulfatase (IDS) | P22304 |
| MPS IIIa - Sanfilippo Type A syndrome | heparan sulfate sulfatase (HSS) or N- | P51688 |
| | sulfoglucosamine sulfohydrolase | |
| | (SGSH) | |
| MPS IIIB - Sanfilippo Type B | N-acetyl-alpha-D-glucosaminidase | P54802 |
| syndrome | (NAGLU) | |
| MPS VI - Maroteaux-Lamy syndrome | arylsulfatase B (ARSB) | P15848 |
| MPS IV A - Morquio syndrome type A | N-acetylgalactosamine-6-sulfatase | P34059 |
| | (GALNS) | |
| MPS IV B - Morquio syndrome type B | Beta-galactosidase 1 (GLB1) | P16278 |
| MPS VII - Sly syndrome | beta-glucuronidase | P08236 |
| MPS VIII | glucosamine-6-sulfate sulfatase | P15586 |
| MPS IX | Hyaluronidase-1 (HYAL1) | Q12794 |
| Multiple Sclerosis | PDCD1 | Q15116 |
| Multiple system atrophy | COQ2 | Q96H96 |
| Myasthenic syndrome, congenital | CHAT | P28329 |
| presynaptic | | |
| Myoclonus | NOL3 | O60936 |
| Myoclonic epilepsy (FAME2) | STARD7 | Q9NQZ5 |
| Narcolepsy | HCRT, OX | O43612 |
| | MOG | Q16653 |
| Neuroacanthocytosis - McLeod | XK | P51811 |
| syndrome | | |
| Neurodevelopmental disorder with | TTC5 | Q8N0Z6 |
| cerebral atrophy and facial | | |
| dysmorphism (NEDCAFD) | | |
| Neurodevelopmental disorder with | NCDN | Q9UBB6 |
| infantile epileptic spasms | | |
| Neurofibromatosis | NF1 | P21359 |
| Neuromyotonia | HINT1 | P49773 |
| Neuronal Ceroid Lipofuscinosis | PPT1 | P50897 |
| | TPP1 | O14773 |
| | CLN5 | O75503 |
| | CLN3 | Q13286 |
| | CLN6 | Q9NWW5 |
| | CLN8 | Q9UBY8 |
| | DNAJC5 | Q9H3Z4 |
| | MFSD8 | Q8NHS3 |
| | CTSD | P07339 |
| Neuropathy, ataxia and retinitis | MTATP6 | P00846 |
| pigmentosa (NARP) | | |
| Neuropathy, hereditary sensory and | WNK1 | Q9H4A3 |
| autonomic, type II | | |
| Neuropathy, hypomyelinating | EGR2 | P11161 |
| congenital 1 | | |
| Niemann-Pick disease | Sphingomyelin phosphodiesterase 1 | P17405 |
| | (SMPD1) | |
| | NPC intracellular cholesterol transporter | O15118 |
| | 1 (NPC1) | |
| Ohtahara Syndrome - Developmental | ARX | Q96QS3 |
| and epileptic encephalopathy 1 | | |
| Omithine Transcarbamylase deficiency | OTC | P00480 |
| Orthostatic intolerance | SLC6A2 | P23975 |
| Parkinson's disease | Glucocerebrosidase (GBA1) | P04062 |
| | Dopamine decarboxylase (DDC) | P20711 |
| | Neurturin | Q99748 |
| | Glial derived growth factor (GDGF) | P39905 |
| | Tyrosine hydroxylase (TH), tyrosine 3- | P07101 |
| | monooxygenase | |
| | Glutamic acid decarboxylase (GAD) | Q99259 |
| | Fibroblast growth factor 2 (FGF2) | P09038 |
| | Brain-derived neurotrophic factor | P23560 |
| | (BDNF) | |
| Paroxysmal Choreoathetosis | PNKD | Q8N490 |
| Pelizaeus-Merzbacher Disease | PLP1 | P60201 |
| Pena-Shokeir Type II Syndrome | ERCC6 | Q03468 |
| Periodic Paralyses | SCN4A | P35499 |
| Phelan-McDermid syndrome | SH3 and multiple ankyrin repeat | Q9BYB0 |
| | domains protein 3 (SHANK3) | |
| Phytanic Acid Storage Disease - | PEX1 | O43933 |
| peroxisome biogenesis disorder 1B | | |
| Pick disease | PSEN1 | A0A024R6A3 |
| | MAPT (tau) | P10636 |
| Porencephaly type 1 | COL4A1 | P02462 |
| Primary Lateral Sclerosis, juvenile | ALS2 | Q96Q42 |
| Primary Progressive Aphasia | GRN | P28799 |
| Progressive external ophtalmoplegia | POLG | P54098 |
| | POLG2 | Q9UHN1 |
| | SLC25A4 | P12235 |
| | TWNK | Q96RR1 |
| Progressive bulbar palsy | SLC52A3 | Q9NQ40 |
| Progressive supranuclear palsy | Microtubule-associated protein tau | P10636 |
| | (MAPT), Tau | |
| Pseudo-Torch syndrome | OCLN | Q16625 |
| | STAT2 | P52630 |
| | USP18 | Q9UMW8 |
| Retinitis Pigmentosa 38 - rod-cone | Tyrosine-protein kinase Mer (MERTK) | Q12866 |
| dystrophy | | |
| Retinitis Pigmentosa 40 | PDE6B | P35913 |
| Rett syndrome | Methyl-CpG-binding protein 2 (MECP2) | P51608 |
| Sandhoff disease | Beta-hexosaminidase subunit alpha | P06865 |
| | (HEXA) | |
| | Beta-hexosaminidase subunit beta | P07686 |
| | (HEXB) | |
| Schizencephaly | SIX3 | O95343 |
| | EMX2 | Q04743 |
| | SHH | Q15465 |
| Seitelberger Disease | PLA2G6 | O60733 |
| Septo-optic dysplasia - De Morsier | HESX1 | Q9UBX0 |
| syndrome | | |
| Snijders Blok-Fisher syndrome | POU3F3 | P20264 |
| Spastic Paraplegias | SPG11 | Q96JI7 |
| | SPAST | Q9UBP0 |
| | KIF5A | Q12840 |
| | NIPA1 | Q7RTP0 |
| | CYP7B1 | O75881 |
| | ATL1 | Q8WXF7 |
| Spinal Muscular Atrophy - Kugelberg- | Survival motor neuron protein (SMN), | Q16637 |
| Welander Disease | SMN1 | |
| Spinocerebellar ataxia | Ataxin-1 (ATXN1), SCA1 | P54253 |
| | Ataxin-2 (ATXN2), SCA2 | Q99700 |
| | Ataxin-3 (ATXN3), SCA3 | P54252 |
| | ZFHX3 | Q15911 |
| | CACNA1A | O00555 |
| | ATXN7, SCA7 | O15265 |
| | TMEM240 | Q5SV17 |
| Sporadic Inclusion Body Myositis | Follistatin (FST) | P19883 |
| Steele-Richardson-Olszewski | MAPT(Tau) | P10636 |
| syndrome - Parkinson-dementia | | |
| syndrome | | |
| Stiff-Person Syndrome, congenital | GLRA1 | P23415 |
| | GLRB | P48167 |
| Striatonigral degeneration | NUP62 | P37198 |
| | PDE8B | O95263 |
| | MTATP6 | P00846 |
| | VAC14 | Q08AM6 |
| Stroke | Tissue-type plasminogen activator (tPA) | P00750 |
| | Neurogenic differentiation factor 1 | Q13562 |
| | (NeuroD1) | |
| Sturge-Weber Syndrome | GNAQ | P50148 |
| Subcortical Vascular Encephalopathy - | HTRA1 | Q92743 |
| Cerebral Arteriopathy | | |
| Systemic Lupus Erythematosus | DNASE1L3 | Q13609 |
| | TLR7 | Q9NYK1 |
| Tardive Dyskinesia | CYP2D6 | P10635 |
| Tay-Sachs disease | Beta-hexosaminidase subunit alpha | P06865 |
| | (HEXA) | |
| Tourette Syndrome | HDC | P19113 |
| | SLITRK1 | Q96PX8 |
| Tremor, hereditary type 1 | DRD3 | P35462 |
| Troyer Syndrome | SPART | Q8N0X7 |
| Tuberous Sclerosis | TSC1 | Q92574 |
| | TSC2 | P49815 |
| | IFNG | P01579 |
| Von Hippel-Lindau Disease | VHL | P40337 |
| | CCND1 | P24385 |
| Von Recklinghausen Disease | NF1 | P21359 |
| Werdnig-Hoffman Disease | SMN1 | Q16637 |
| West Syndrome, X-linked | ARX | Q96QS3 |
| Wilson disease | ATP7B | P35670 |
| Wolman's disease - acid lipase | LIPA | P38571 |
| disease | | |
| X-linked adrenoleukodystrophy | ATP-binding cassette sub-family D | P33897 |
| | member 1 (ABCD1) | |
| X-linked Retinoschisis | Retinoschisin (RS1) | O15537 |
| X-Linked Retinitis Pigmentosa | X-linked retinitis pigmentosa GTPase | Q92834 |
| | regulator (RPGR) | |
| X-Linked Spinal and Bulbar Muscular | UBA1 | P22314 |
| Atrophy | | |

In some embodiments, the payload is an antisense oligonucleotide effective in treating a CNS disease, for example by modulating expression of a target gene. Exemplary CNS diseases which may be treated using an antisense oligonucleotide include amyotrophic lateral sclerosis, Huntington's disease, and Alzheimer's disease. Exemplary target genes of such antisense oligonucleotides for treatment of CNS disease include SOD, C9orf72, Ataxin 2, huntingtin (HTT), Sortilin-related receptor, Microtubule-Associated Protein Tau (MAPT), and neutral sphingomyelinase (N-SMase). Various antisense oligonucleotides effective in treatment of CNS diseases are described in the art and include, for example, those described in Bennett et al., 2019 Annu Rev Neurosci. 42:385-406, Rinaldi et al., 2018, Nature Reviews Neurology, 14:9-21, and Rook et al., 2022, BioDrugs 36(2):105-119, each incorporated herein by reference.

Non-limiting, example antisense oligonucleotides that can be delivered via the viral particles of the disclosure (along with associated target genes and CNS tissue related diseases that can be treated) and are provided in Table C2. Where such antisense oligonucleotides are indicated by reference to a patent or patent application publication, such patent and patent applications are incorporated herein by reference in their entirety for their disclosed antisense oligonucleotide structures and sequences.

TABLE C2
Exemplary CNS Indications, Target Genes, and Antisense Oligonucleotide Transgenes

| | | Uniprot | |
|---|---|---|---|
| | | Accession | |
| Indication | Target Gene | # (Human) | Exemplary transgene(s) |
| Amyotrophic | Superoxide | P00441 | Antisense oligonucleotides described in |
| lateral sclerosis | dismutase 1 (SOD 1) | | U.S. Patent Application Publication Nos. |
| | | | 2024/0182903 A1, 2022/0073930 A1, |
| | | | 2017/0152517 A1, 2020/0354723 A1, |
| | | | 2022/0315930 A1, and 2023/0193281 A1. |
| | C9orf72 | Q96LT7 | Antisense oligonucleotides described in |
| | | | U.S. Patent Application Publication Nos. |
| | | | 2018/0023077 A1, 2015/0267197 A1, |
| | | | 2020/0385723 A1, 2016/0237432 A1, |
| | | | 2023/0098111 A1, and 20190231808 A1. |
| Huntington's | Huntingtin (HTT) | P42858 | Antisense oligonucleotides described in: |
| disease | | | U.S. Patent Application Publication Nos. |
| | | | 20140303238 A1, 2017/0253877 A1, and |
| | | | 2022/0098585 A1; and |
| | | | PCT Patent Application Publication No. |
| | | | WO/2024/035946 A1. |
| Alzheimer's | Sortilin-related | Q92673 | Antisense oligonucleotides described in |
| disease | receptor (SORL1) | | PCT Patent Application Publication No. |
| | | | WO/2023/275376 A1. |
| | Neutral | O60906 | Antisense oligonucleotides described in |
| | sphingomyelinase | | U.S. Patent Application Publication No. |
| | (N-SMase), also | | 2014/0275210 A1. |
| | known as | | |
| | Sphingomyelin | | |
| | phosphodiesterase 2 | | |
| | (SMPD2) | | |
| Spinocerebellar | Ataxin-2 (ATXN2) | Q99700 | Antisense oligonucleotides described in |
| ataxia type 2 | | | U.S. Patent Application Publication No. |
| (SCA2) | | | 2020/0024600 A1 |
| Parkinson's | SNCA | P37840 | Antisense oligonucleotides described in |
| Disease | | | U.S. Patent Application Publication Nos. |
| | | | 20050137155 A1, 20050186591 A1, |
| | | | 20200362347 A1, 20040219671 A1, |
| | | | 20220119811 A1, 20210180065 A1, and |
| | | | 20200392494 A1 |

The disclosure is further directed, in part, to a method of delivering a payload to a subject, e.g., an animal or human subject. In some embodiments, a method of delivering a payload to a subject comprises administering to the subject a dependoparvovirus particle comprising a variant polypeptide (e.g., described herein) comprising the payload, e.g., in a quantity and for a time sufficient to deliver the payload. In some embodiments, the dependoparvovirus particle is a dependoparvovirus particle described herein and comprises a payload described herein. In some embodiments, the particle delivers the payload to the CNS. In some embodiments, the delivery to the CNS is increased as compared to a particle without the variant capsid polypeptide or as compared to a wild-type capsid polypeptide, e.g., a particle with capsid polypeptides of SEQ ID NO:7. In some embodiments, the particle delivers the payload to skeletal muscle tissue. In some embodiments, the delivery to the skeletal muscle tissue is increased as compared to a particle without the variant capsid polypeptide or as compared to a wild-type capsid polypeptide, e.g., a particle with capsid polypeptides of SEQ ID NO:7.

In some embodiments, the particle delivers the payload to muscle tissue (e.g., cardiac muscle or skeletal muscle). In some embodiments, the delivery to muscle is increased as compared to a particle without the variant capsid polypeptide or as compared to a wild-type capsid polypeptide, e.g., a particle with capsid polypeptides of SEQ ID NO:7.

In some embodiments, virus particles comprising a genome are provided, wherein the genome includes a nucleic acid expression construct including a heterologous transgene and one or more regulatory elements, where the one or more regulatory elements include a muscle specific promoter. Examples of muscle specific promoters that can be used to drive expression of a transgene in muscle include but are not limited to Desmin (DES), CAMK, Mb, myosin (e.g., myo-3), dystrophin, muscle creatine kinase (MCK), MHCK7, CK6, CK7, CK8, CK8e, dMCK, tMCK, MH, SPc-5-12 (also known as C5-12), alpha skeletal actin (ASKA), SP-301, E-syn, myosin light chain (MLC), myosin heavy chain (MHC), four and a half LIM domains protein 1 (FHL1), alpha 2 actinin (ACTN2), filamin-C (FLNC), sarcoplasmic/endoplasmic reticulum calcium ATPase 1 (ATP2A1), troponin I type 1 (TNN11), myosin-1 (MYH1), phosphorylatable, fast skeletal muscle myosin light chain (MYLPF), alpha-3 chain tropomyosin (TPM3), Pitx3, and ankyrin repeat domain-containing protein 2 (ANKRD2) promoters. Promoters capable of driving expression in muscle are further described in Skopenkova et al., 2021, Acta Naturae 13(1):47-58, Piekarowicz et al., 2019, Mol Ther Methods Clin Dev. 15:157-169, Wang, 2008 Gene Ther. 15:1489-1499, Coulon et al., 2007, JBC 282(45):33192-33200, WO 2021/127655, and WO 2023/006890, the contents of each of which are incorporated herein by reference in their entireties.

In some aspects, the payload comprises a molecule that is effective in treating a muscle disease, such as, for example, a protein or an antisense oligonucleotide such as an RNA interference nucleotide (e.g., shRNA, siRNA or miRNA).

In certain aspects, the disclosure provides a virus particle comprising (a) a capsid polypeptide described herein, for example, a capsid polypeptide described in Section 6.2 and (b) an engineered viral genome comprising (i) a nucleotide sequence encoding a molecule for the treatment of Duchenne muscular dystrophy (DMD), for example human Dystrophin (DMD) or a fragment or variant thereof (e.g., a variant thereof having at least 90% or at least 95% sequence identity to human DMD), and (ii) a promoter operably linked to said nucleotide sequence. An exemplary accession number for human DMD is set forth in Table M1. The viral genome can further comprise one or more (e.g., two, three, four, or all five) of (a) a pair of dependoparvovirus ITRs, (b) an intron, (c) an enhancer or repressor sequence, (d) a stuffer sequence, and (e) a polyA sequence. Preferably, the viral genome comprises ITRs flanking the nucleotide sequence and a polyA sequence operably linked to the nucleotide sequence. Typically, the viral genome lacks rep and cap sequences, which are in trans by the host cell in which the virus particle is produced. In some embodiments, the viral genome is self-complementary.

In certain aspects, the disclosure provides a virus particle comprising (a) a capsid polypeptide described herein, for example, a capsid polypeptide described in Section 6.2 and (b) an engineered viral genome comprising (i) a nucleotide sequence encoding a molecule for the treatment of X-linked myotubular myopathy, for example human myotubularin (MTM1) or a fragment or variant thereof (e.g., a variant thereof having at least 90% or at least 95% sequence identity to human MTM1), and (ii) a promoter operably linked to said nucleotide sequence. An exemplary accession number for human MTM1 is set forth in Table M1. The viral genome can further comprise one or more (e.g., two, three, four, or all five) of (a) a pair of dependoparvovirus ITRs, (b) an intron, (c) an enhancer or repressor sequence, (d) a stuffer sequence, and (e) a polyA sequence. Preferably, the viral genome comprises ITRs flanking the nucleotide sequence and a polyA sequence operably linked to the nucleotide sequence. Typically, the viral genome lacks rep and cap sequences, which are in trans by the host cell in which the virus particle is produced. In some embodiments, the viral genome is self-complementary.

In certain aspects, the disclosure provides a virus particle comprising (a) a capsid polypeptide described herein, for example, a capsid polypeptide described in Section 6.2 and (b) an engineered viral genome comprising (i) a nucleotide sequence encoding a molecule for the treatment of Myotonic Dystrophy Type 1 (DM1), for example human myotonin-protein kinase (DMPK) or a fragment or variant thereof (e.g., a variant thereof having at least 90% or at least 95% sequence identity to human DMPK, and (ii) a promoter operably linked to said nucleotide sequence. An exemplary accession number for human DMPK is set forth in Table M1. The viral genome can further comprise one or more (e.g., two, three, four, or all five) of (a) a pair of dependoparvovirus ITRs, (b) an intron, (c) an enhancer or repressor sequence, (d) a stuffer sequence, and (e) a polyA sequence. Preferably, the viral genome comprises ITRs flanking the nucleotide sequence and a polyA sequence operably linked to the nucleotide sequence. Typically, the viral genome lacks rep and cap sequences, which are in trans by the host cell in which the virus particle is produced. In some embodiments, the viral genome is self-complementary.

In certain aspects, the disclosure provides a virus particle comprising (a) a capsid polypeptide described herein, for example, a capsid polypeptide described in Section 6.2 and (b) an engineered viral genome comprising (i) a nucleotide sequence encoding a molecule for the treatment of Limb-girdle muscular dystrophy (LGMD), for example human Gamma-sarcoglycan, Alpha-sarcoglycan, Beta-sarcoglycan, or Delta-sarcoglycan or a fragment or variant thereof (e.g., a variant thereof having at least 90% or at least 95% sequence identity to human Gamma-sarcoglycan, Alpha-sarcoglycan, Beta-sarcoglycan, or Delta-sarcoglycan), and (ii) a promoter operably linked to said nucleotide sequence. Exemplary accession numbers for human Gamma-sarcoglycan, Alpha-sarcoglycan, Beta-sarcoglycan, and Delta-sarcoglycan are set forth in Table M1. The viral genome can further comprise one or more (e.g., two, three, four, or all five) of (a) a pair of dependoparvovirus ITRs, (b) an intron, (c) an enhancer or repressor sequence, (d) a stuffer sequence, and (e) a polyA sequence. Preferably, the viral genome comprises ITRs flanking the nucleotide sequence and a polyA sequence operably linked to the nucleotide sequence. Typically, the viral genome lacks rep and cap sequences, which are in trans by the host cell in which the virus particle is produced. In some embodiments, the viral genome is self-complementary.

In certain aspects, the disclosure provides a virus particle comprising (a) a capsid polypeptide described herein, for example, a capsid polypeptide described in Section 6.2 and (b) an engineered viral genome comprising (i) a nucleotide sequence encoding a molecule for the treatment of Pompe disease, for example human acid alpha-glucosidase (GAA) or a fragment (e.g., exon 2) or variant thereof (e.g., a variant thereof having at least 90% or at least 95% sequence identity to human GAA or a portion thereof), and (ii) a promoter operably linked to said nucleotide sequence. An exemplary accession number for human GAA is set forth in Table M1. The viral genome can further comprise one or more (e.g., two, three, four, or all five) of (a) a pair of dependoparvovirus ITRs, (b) an intron, (c) an enhancer or repressor sequence, (d) a stuffer sequence, and (e) a polyA sequence. Preferably, the viral genome comprises ITRs flanking the nucleotide sequence and a polyA sequence operably linked to the nucleotide sequence. Typically, the viral genome lacks rep and cap sequences, which are in trans by the host cell in which the virus particle is produced. In some embodiments, the viral genome is self-complementary.

In certain aspects, the disclosure provides a virus particle comprising (a) a capsid polypeptide described herein, for example, a capsid polypeptide described in Section 6.2 and (b) an engineered viral genome comprising (i) a nucleotide sequence encoding a molecule for the treatment of Facioscapulohumeral muscular dystrophy (FSHD), for example an antisense oligonucleotide targeting human DUX4, and (ii) a promoter operably linked to said nucleotide sequence. The viral genome can further comprise one or more (e.g., two, three, four, or all five) of (a) a pair of dependoparvovirus ITRs, (b) an intron, (c) an enhancer or repressor sequence, (d) a stuffer sequence, and (e) a polyA sequence. Preferably, the viral genome comprises ITRs flanking the nucleotide sequence and a polyA sequence operably linked to the nucleotide sequence. Typically, the viral genome lacks rep and cap sequences, which are in trans by the host cell in which the virus particle is produced. In some embodiments, the viral genome is self-complementary.

In certain aspects, the disclosure provides a virus particle comprising (a) a capsid polypeptide described herein, for example, a capsid polypeptide described in Section 6.2 and (b) an engineered viral genome comprising (i) a nucleotide sequence encoding a molecule for the treatment of Facioscapulohumeral muscular dystrophy (FSHD), for example human SMCHD1 or a fragment or variant thereof (e.g., a variant thereof having at least 90% or at least 95% sequence identity to human SMCHD1 or a portion thereof), and (ii) a promoter operably linked to said nucleotide sequence. An exemplary accession number for human SMCHD1 is set forth in Table M1. The viral genome can further comprise one or more (e.g., two, three, four, or all five) of (a) a pair of dependoparvovirus ITRs, (b) an intron, (c) an enhancer or repressor sequence, (d) a stuffer sequence, and (e) a polyA sequence. Preferably, the viral genome comprises ITRs flanking the nucleotide sequence and a polyA sequence operably linked to the nucleotide sequence. Typically, the viral genome lacks rep and cap sequences, which are in trans by the host cell in which the virus particle is produced. In some embodiments, the viral genome is self-complementary.

Exemplary muscle tissue related diseases that can be treated include but are not limited to Acid Maltase Deficiency (AMD), Amyotrophic Lateral Sclerosis (ALS), Andersen-Tawil Syndrome, Barth syndrome (TAZ), Becker Muscular Dystrophy (BMD), Becker Myotonia Congenita, Bethlem Myopathy, Bulbospinal Muscular Atrophy (Spinal-Bulbar Muscular Atrophy), Carnitine Deficiency, Carnitine Palmityl Transferase Deficiency (CPT Deficiency), Central Core Disease (CCD), Centronuclear Myopathy, Charcot-Marie-Tooth Disease (CMT), Congenital Muscular Dystrophy (CMD), Congenital Myasthenic Syndromes (CMS), Congenital Myotonic Dystrophy, Cori Disease (Debrancher Enzyme Deficiency), Danon disease, Debrancher Enzyme Deficiency, Dejerine-Sottas Disease (DSD), Dermatomyositis (DM), Distal Muscular Dystrophy (DD), Distal myopathy with anterior tibial onset, Duchenne Muscular Dystrophy (DMD), Dystrophia Myotonica (Myotonic Muscular Dystrophy), Emery-Dreifuss Muscular Dystrophy (EDMD), Endocrine Myopathies, Eulenberg Disease (Paramyotonia Congenita), Facioscapulohumeral Muscular Dystrophy (FSH or FSHD), Finnish (Tibial) Distal Myopathy, Forbes Disease (Debrancher Enzyme Deficiency), Friedreich's Ataxia (FA), Fukuyama Congenital Muscular Dystrophy, Glycogenosis Type 10, Glycogenosis Type 11, Glycogenosis Type 2, Glycogenosis Type 3, Glycogenosis Type 5, Glycogenosis Type 7, Glycogenosis Type 9, Gowers-Laing Distal Myopathy, Hauptmann-Thanheuser MD (Emery-Dreifuss Muscular Dystrophy), Hereditary Inclusion-Body Myositis, Hereditary Motor and Sensory Neuropathy (Charcot-Marie-Tooth Disease), Hyperthyroid Myopathy, Hypothyroid Myopathy, Inclusion-Body Myositis (IBM), Inherited Myopathies, Integrin-Deficient Congenital Muscular Dystrophy, Kennedy Disease (Spinal-Bulbar Muscular Atrophy), Kugelberg-Welander Disease (Spinal Muscular Atrophy), Lactate Dehydrogenase Deficiency, Lambert-Eaton Myasthenic Syndrome (LEMS), Limb-Girdle Muscular Dystrophy (LGMD), Lou Gehrig's Disease (Amyotrophic Lateral Sclerosis), McArdle Disease (Phosphorylase Deficiency), Merosin-Deficient Congenital Muscular Dystrophy, Metabolic Diseases of Muscle, Mitochondrial Myopathy, Miyoshi myopathy, Miyoshi Distal Myopathy, Motor Neurone Disease, Muscle-Eye-Brain Disease, Myasthenia Gravis (MG), Myoadenylate Deaminase Deficiency, Myofibrillar Myopathy, Myophosphorylase Deficiency, Myotonia Congenita (MC), Myotonic Muscular Dystrophy (MMD), Myotubular Myopathy (MTM or MM), Nemaline Myopathy, Nonaka Distal Myopathy, Oculopharyngeal Muscular Dystrophy (OPMD), Paramyotonia Congenita, Pearson Syndrome, Periodic Paralysis, Peroneal Muscular Atrophy (Charcot-Marie-Tooth Disease), Phosphofructokinase Deficiency, Phosphoglycerate Kinase Deficiency, Phosphoglycerate Mutase Deficiency, Phosphorylase Deficiency, Phosphorylase Deficiency, Polymyositis (PM), Pompe Disease (Acid Maltase Deficiency), Primary merosin deficiency (LAMA2), Progressive External Ophthalmoplegia (PEO), Rod Body Disease (Nemaline Myopathy), Spinal Muscular Atrophy (SMA), Spinal-Bulbar Muscular Atrophy (SBMA), Steinert Disease (Myotonic Muscular Dystrophy), Tarui Disease (Phosphofructokinase Deficiency), Thomsen Disease (Myotonia Congenita), Ullrich Congenital Muscular Dystrophy, Walker-Warburg Syndrome (Congenital Muscular Dystrophy), Welander Distal Myopathy, Werdnig-Hoffmann Disease (Spinal Muscular Atrophy), and ZASP-Related Myopathy.

Payloads suitable for treating muscle-related disease are known in the art and include the following disease (suitable payload) combinations: Barth syndrome (TAZ), Primary merosin deficiency (LAMA2), Duchenne muscular dystrophy (DMD), Becker muscular dystrophy (BMD), Danon disease (LAMP2), Limb girdle muscular dystrophy (Subtypes and affected genes: LGMD1A (TTID), LGMD1B (LMNA), LGMD1C (CAV3), LGMD1D (DNAJB6), LGMD1E (DES), LGMD1F (TNPO3), LGMD1G (HNRPDL), LGMD1H, LGMD2A (CAPN3), LGMD2B (DYSF), LGMD2C (SGCG), LGMD2D (SGCA), LGMD2E (SGCB), LGMD2F (SGCD), LGMD2G (TCAP), LGMD2H (TRIM32), LGMD2I (FKRP), LGMD2J (TTN), LGMD2K (POMT1), LGMD2L (AN05), LGMD2M (FKTN), LGMD2N (POMT2), LGMD20 (POMGNT1), LGMD2Q (PLEC1)), Miyoshi myopathy (DYSF), Distal myopathy with anterior tibial onset (DYSF), Welander distal myopathy (TIA1), Gowers-Laing distal myopathy (MYH7), Facioscapulohumeral muscular dystrophy (Subtypes and affected genes: Type 1 (DUX4), Type 2 (SMCHD1)), Oculopharyngeal muscular dystrophy (PABPN1), myotonic dystrophy (Subtypes and affected genes: DM1 (DMPK) and DM2 (ZNF9)), congenital myotonia (CLCN1), paramyotonia congenital (SCN4A), myotubular myopathy (MTM1), glycogen storage disease type II (Pompe disease) (GAA).

In some embodiments, the payload is selected from: TAZ, LAMA2, DMD, LAMP2, CAPN3, DYSF, SGCA, SGCB, FKRP, PABPN1, MTM1, and GAA.

Certain exemplary muscle tissue related diseases that can be treated and exemplary heterologous transgenes that can be delivered via the viral particles of the disclosure are provided in Table M1.

TABLE M1
Exemplary Muscle Indications and Transgenes

| | | UniProt |
|---|---|---|
| | | Accession # |
| Indication | Transgene | (Human) |
| Acid maltase deficiency - Glycogen | GAA | P10253 |
| storage disease II | | |
| Advanced heart failure | SERCA2a, ATP2A2 | P16615 |
| Amyotrophic lateral sclerosis (ALS) - | Superoxide dismutase-1 (SOD1) | P00441 |
| Lou Gehrig's disease | | |
| Andersen-Tawil Syndrome | KCNJ2 | P63252 |
| Barth syndrome | TAFAZZIN | Q16635 |
| Becker Muscular Dystrophy (BMD) | DMD | P11532 |
| Becker Myotonia Congenita | CLCN1 | P35523 |
| Bethlem Myopathy | COL6A3 | P12111 |
| | COL6A2 | P12110 |
| | COL6A1 | P12109 |
| Bulbospinal Muscular Atrophy | AR | P10275 |
| Carnitine Deficiency, systemic primary | SLC22A5 | O76082 |
| Carnitine Palmityl Transferase | CPT1A | P50416 |
| Deficiency, type 1 | | |
| Carnitine Palmityl Transferase | CPT2 | P23786 |
| Deficiency, type 2 | | |
| Catecholaminergic polymorphic | Calsequestrin-2 (CASQ2) | O14958 |
| ventricular tachycardia 2 (CPVT2) | | |
| Central Core Disease - congenital | RYR1 | P21817 |
| myopathy, type 1A | | |
| Centronuclear Myopathy type 1 | MTMR14 | Q8NCE2 |
| | DNM2 | O14717 |
| Charcot-Marie-Tooth disease | PMP22 | Q01453 |
| | MPZ | P25189 |
| | DNM2 | P50570 |
| | MFN2 | O95140 |
| | KIF1B | O60333 |
| | SBF2 | Q86WG5 |
| | PNKP | Q96T60 |
| | GDAP1 | Q8TB36 |
| | LMNA | P02545 |
| | FGD4 | Q96M96 |
| | MTMR2 | Q13614 |
| Congenital Muscular Dystrophy - | COL6A3 | P12111 |
| Ullrich disease | COL6A2 | P12110 |
| | COL6A1 | P12109 |
| Congenital Myasthenic Syndromes | COLQ | Q9Y215 |
| | AGRN | O00468 |
| | RAPSN | Q13702 |
| | GFPT1 | Q06210 |
| | SCN4A | P35499 |
| | ALG2 | Q9H553 |
| | ALG14 | Q96F25 |
| | DPAGT1 | Q9H3H5 |
| | CHRNE | Q04844 |
| | CHRNA1 | P02708 |
| | DOK7 | Q18PE1 |
| | CHAT | P28329 |
| Congenital Myopathy | ACTA1 | P68133 |
| | STAC3 | Q96MF2 |
| | TPM3 | P06753 |
| Congenital Myotonic Dystrophy | DMPK | Q09013 |
| Cori Disease - Debrancher Enzyme | AGL | P35573 |
| Deficiency - Forbes Disease | | |
| Danon disease | LAMP2 | P13473 |
| Dejerine-Sottas Disease | MPZ | P25189 |
| | EGR2 | P11161 |
| | PMP22 | Q01453 |
| | PRX | Q9BXM0 |
| Distal Muscular Dystrophy, Welander | TIA1 | P31483 |
| Distal Muscular Dystrophy, Miyoshi | DYSF | O75923 |
| Distal myopathy with anterior tibial | DYSF | O75923 |
| onset | | |
| Duchenne Muscular Dystrophy | Dystrophin (DMD) | P11532 |
| | GALGT2, B4GALNT2 | Q8NHY0 |
| Dysferlinopathies | Dysferlin (DYSF) | O75923 |
| Emery-Dreifuss Muscular Dystrophy | EMD | P50402 |
| | SYNE1 | Q8NF91 |
| | SYNE2 | Q8WXH0 |
| | TMEM43 | Q9BTV4 |
| | LMNA | P02545 |
| Eulenberg Disease - Paramyotonia | SCN4A | P35499 |
| Congenita | | |
| Facioscapulohumeral Muscular | SMCHD1 | A6NHR9 |
| Dystrophy | LRIF1 | Q5T3J3 |
| Friedreich's ataxia | Frataxin (FXN) | Q16595 |
| Fukuyama Congenital Muscular | FKTN | O75072 |
| Dystrophy | | |
| Glycogenosis Type 10 - Glycogen | PGAM2 | P15259 |
| storage disease X | | |
| Glycogenosis Type 11 - Glycogen | LDHA | P00338 |
| storage disease XI | | |
| Glycogenosis Type 2 - Glycogen | GAA | P10253 |
| storage disease II - Pompe Disease - | | |
| Acid maltase deficiency | | |
| Glycogenosis Type 3 - Glycogen | AGL | P35573 |
| storage disease III | | |
| Glycogenosis Type 5 - Glycogen | PYGM | P11217 |
| storage disease V - McArdle Disease - | | |
| Myophosphorylase Deficiency | | |
| Glycogenosis Type 7 - Glycogen | PFKM | P08237 |
| storage disease VII - | | |
| Phosphofructokinase Deficiency - Tarui | | |
| Disease | | |
| Glycogenosis Type 9 - Glycogen | PHKB | Q93100 |
| storage disease IX | PHKA2 | P46019 |
| Hereditary Inclusion-Body Myositis | GNE | Q9Y223 |
| Integrin-Deficient Congenital Muscular | ITGA7 | Q13683 |
| Dystrophy | | |
| Kennedy Disease - Spinal-Bulbar | Androgen receptor (AR) | P10275 |
| Muscular Atrophy | | |
| Kugelberg-Welander Disease | SMN1 | Q16637 |
| Lactate dehydrogenase A deficiency | LDHA | P00338 |
| Lactate Dehydrogenase B Deficiency | LDHB | P07195 |
| Lambert-Eaton Myasthenic Syndrome | CACNB2 | Q08289 |
| Laing Distal Myopathy | MYH7 | A7E2Y1 |
| Limb Girdle Muscular Dystrophy Type | Gamma-sarcoglycan | Q13326 |
| 2C (LGMD-2C) | | |
| Limb Girdle Muscular Dystrophy Type | Alpha-sarcoglycan | Q16586 |
| 2D (LGMD-2D) | | |
| Limb Girdle Muscular Dystrophy | Beta-sarcoglycan | Q16585 |
| Type2E (LGMD-2E) | | |
| Limb Girdle Muscular Dystrophy Type | Delta-sarcoglycan | Q92629 |
| 2F (LGMD-2F) | | |
| Merosin-Deficient Congenital Muscular | LAMA2 | P24043 |
| Dystrophy | | |
| Muscle-Eye-Brain Disease | POMGNT1 | Q8WZA1 |
| Mitochondrial Myopathy | CHCHD10 | Q8WYQ3 |
| Miyoshi myopathy | DYSF | O75923 |
| Myoadenylate Deaminase Deficiency | AMPD1 | P23109 |
| Myofibrillar Myopathy 1 | DES | P17661 |
| Myofibrillar Myopathy 2 | CRYAB | P02511 |
| Myofibrillar Myopathy 3 | MYOT | Q9UBF9 |
| Myofibrillar Myopathy 4 - ZASP related | LDB3, ZASP | O75112 |
| myopathy | | |
| Myofibrillar Myopathy 5 | FLNC | Q14315 |
| Myofibrillar Myopathy 6 | BAG3 | O95817 |
| Myofibrillar Myopathy 7 | KY | Q8NBH2 |
| Myofibrillar Myopathy 8 | PYROXD1 | Q8WU10 |
| Myofibrillar Myopathy 9 | TTN | Q8WZ42 |
| Myofibrillar Myopathy 10 | SVIL | O95425 |
| Myofibrillar Myopathy 11 | UNC45B | Q8IWX7 |
| Myofibrillar Myopathy 12 | MYL2 | Q99972 |
| Myotonic dystrophy Type 1 - Steinert | Myotonin-protein kinase (DMPK) | Q09013 |
| Disease | | |
| Myotonic dystrophy Type 2 | CNBP | P62633 |
| Myotubular Myopathy | MTM1 | Q13496 |
| Nemaline Myopathy 1 | TPM3 | P06753 |
| Nemaline Myopathy 2 | NEB | P20929 |
| Nemaline Myopathy 5A, 5B, 5C | TNNT1 | P13805 |
| Nemaline Myopathy 3 | ACTA1 | P68133 |
| Nemaline Myopathy 6 | KBTBD13 | C9JR72 |
| Nemaline Myopathy 4 | TPM2 | P07951 |
| Nemaline Myopathy 7 | CFL2 | Q9Y281 |
| Nemaline Myopathy 8 | KLHL40 | Q2TBA0 |
| Nemaline Myopathy 9 | KLHL41 | O60662 |
| Nemaline Myopathy 10 | LMOD3 | Q0VAK6 |
| Nonaka Distal Myopathy | GNE | Q9Y223 |
| Oculopharynggeal muscular dystrophy | PABPN1 | Q86U42 |
| Omithine Transcarbamylase deficiency | OTC | P00480 |
| Paramyotonia Congenita | SCN4A | P35499 |
| Periodic Paralysis, hypokalemic | CACNA1S | Q13698 |
| Periodic Paralysis, hyperkalemic | SCN4A | P35499 |
| Phosphoglycerate Kinase Deficiency | PGK1 | P00558 |
| Polymyositis | PMSCL2 | Q01780 |
| | PMSCL1 | Q06265 |
| Progressive External Ophthalmoplegia | POLG | P54098 |
| | POLG2 | Q9UHN1 |
| | SLC25A4 | P12235 |
| | TWNK | Q96RR1 |
| Spinal Muscular Atrophy type 3 - | Survival motor neuron protein (SMN), | Q16637 |
| Kugelberg-Welander Disease | SMN1 | |
| Thomsen Disease - Myotonia | CLCN1 | P35523 |
| Congenita (autosomal dominant) | | |
| Walker-Warburg Syndrome | POMT1 | Q9Y6A1 |
| X-linked myotubular myopathy | Myotubularin (MTM1) | Q13496 |
| Werdnig-Hoffmann Disease - Spinal | SMN1 | Q16637 |
| Muscular Atrophy type 1 | | |

In some embodiments, the payload is human dystrophin.

In some embodiments, the payload is an antisense oligonucleotide effective in treating a muscle disease, for example by modulating expression of a target gene. Exemplary target genes of such antisense oligonucleotides for treatment of muscle disease include, for example, MSTN, INHBA, ACVR1B, MLCK1, ACVR1, FBXO32, TRIM63, MEF2D, KLF15, MED1, MED13, DUX4, LMNA, SMN, GAA, GYS2, DMD, DMPK, MSTN, and PPP1R3A.

Non-limiting, example antisense oligonucleotides that can be delivered via the viral particles of the disclosure (along with associated target genes and muscle tissue related diseases that can be treated) and are provided in Table M2. Where such antisense oligonucleotides are indicated by reference to a patent or patent application publication, such patent and patent applications are incorporated herein by reference in their entirety for their disclosed antisense oligonucleotide structures and sequences.

TABLE M-2
Exemplary Muscle Indications, Target Genes, and Antisense Oligonucleotide Transgenes

| | | Uniprot Accession | |
|---|---|---|---|
| Indication | Target Gene | # (Human) | Exemplary transgene(s) |
| Hutchinson-Gilford | lamin A (LMNA) | P02545 | Antisense oligonucleotides described in: |
| progeria syndrome | | | U.S. Pat. No. 10,076,536 and in |
| (HGPS) | | | U.S. Patent Application Publication Nos. |
| | | | 2017/0051278 A1, 2018/0271893 A1 |
| Spinal Muscular | Survival Motor | Q16637 | Antisense oligonucleotides described in |
| Atrophy (SMA) | Neuron (SMN) | | U.S. Patent Application Publication No. |
| | | | 2019/0015440 A1 |
| Glycogen storage | acid alpha- | P10253 | Antisense oligonucleotides described in |
| disease type II | glucosidase | | U.S. Patent Application Publication No. |
| (GSD-II), or Pompe | (GAA) | | 2018/0216111 A1 |
| disease | Glycogen | P54840 | Antisense oligonucleotides described in |
| | synthase (GYS2) | | U.S. Patent Application Publication No. |
| | | | 2017/0182189 A1 |
| Duchenne muscular | Dystrophin | P11532 | Antisense oligonucleotides described in: |
| dystrophy | (DMD) | | U.S. Patent Application Publication Nos. |
| | | | 2019/0177723 A1 and 20210261963 A1; and |
| | | | PCT Patent Application Publication Nos. |
| | | | WO 2017/062835 A2 and 2019/059973 A1 |
| Myotonic dystrophy | Dystrophia | Q09013 | Antisense oligonucleotides described in |
| Type 1 - Steinert | Myotonica- | | U.S. Patent Application Publication Nos. |
| Disease | Protein Kinase | | 2024/0117356 A1, 2010/0016215 A1, |
| | (DMPK) | | 2013/0237585 A1, 2015/0064181 A1, |
| | | | 2015/0238627 A1, and 2016/0304877 A1 |
| Muscle atrophy | Myostatin | O14793 | Antisense oligonucleotides described in |
| | (MSTN) | | U.S. Patent Application Publication No. |
| | | | 2018/0327749 A1 |
| Facioscapulohumeral | Double | Q9UBX2 | Antisense oligonucleotides described in |
| Muscular Dystrophy | homeobox 4 | | U.S. Patent Application Publication No. |
| | (DUX4) | | 2023/0272065 A1 |

### 6.7.2. Methods of Treatment

The disclosure is directed, in part, to a method of treating a disease or condition in a subject, e.g., an animal or human subject. In some embodiments, a method of treating a disease or condition in a subject comprises administering to the subject a dependoparvovirus particle comprising a variant polypeptide described herein, e.g., comprising a payload described herein. In some embodiments, the dependoparvovirus particle, which comprises a variant polypeptide, comprising a payload described herein is administered in an amount and/or time effective to treat the disease or condition. In some embodiments, the payload is a therapeutic product. In some embodiments, the payload is a nucleic acid, e.g., encoding an exogenous polypeptide. The disclosure is also directed to a dependoparvovirus particle comprising a variant polypeptide described herein, e.g., comprising a payload described herein, for use in the methods of treatment described herein. The disclosure is also directed to the use of a dependoparvovirus particle comprising a variant polypeptide described herein, e.g., comprising a payload described herein for the manufacture of a medicament for the treatment of a disease or condition as described herein.

The dependoparvovirus particles comprising a variant polypeptide described herein or produced by the methods described herein can be used to express one or more therapeutic proteins to treat various diseases or disorders. In some embodiments, the disease or disorder is a cancer, e.g., a cancer such as carcinoma, sarcoma, leukemia, lymphoma; or an autoimmune disease, e.g., multiple sclerosis. Non-limiting examples of carcinomas include esophageal carcinoma; bronchogenic carcinoma; colon carcinoma; colorectal carcinoma; gastric carcinoma; hepatocellular carcinoma; basal cell carcinoma, squamous cell carcinoma (various tissues); bladder carcinoma, including transitional cell carcinoma; lung carcinoma, including small cell carcinoma and non-small cell carcinoma of the lung; adrenocortical carcinoma; sweat gland carcinoma; sebaceous gland carcinoma; thyroid carcinoma; pancreatic carcinoma; breast carcinoma; ovarian carcinoma; prostate carcinoma; adenocarcinoma; papillary carcinoma; papillary adenocarcinoma; cystadenocarcinoma; medullary carcinoma; renal cell carcinoma; uterine carcinoma; testicular carcinoma; osteogenic carcinoma; ductal carcinoma in situ or bile duct carcinoma; choriocarcinoma; seminoma; embryonal carcinoma; Wilm's tumor; cervical carcinoma; epithelial carcinoma; and nasopharyngeal carcinoma. Non-limiting examples of sarcomas include fibrosarcoma, myxosarcoma, liposarcoma, angiosarcoma, endotheliosarcoma, lymphangiosarcoma, chondrosarcoma, chordoma, osteogenic sarcoma, osteosarcoma, lymphangioendotheliosarcoma, synovioma, mesothelioma, Ewing's sarcoma, leiomyosarcoma, rhabdomyosarcoma, and other soft tissue sarcomas. Non-limiting examples of solid tumors include ependymoma, pinealoma, hemangioblastoma, acoustic neuroma, oligodendroglioma, glioma, astrocytoma, medulloblastoma, craniopharyngioma, menangioma, melanoma, neuroblastoma, and retinoblastoma. Non-limiting examples of leukemias include chronic myeloproliferative syndromes; T-cell CLL prolymphocytic leukemia, acute myelogenous leukemias; chronic lymphocytic leukemias, including B-cell CLL, hairy cell leukemia; and acute lymphoblastic leukemias. Examples of lymphomas include, but are not limited to, B-cell lymphomas, such as Burkitt's lymphoma; and Hodgkin's lymphoma. In some embodiments, the disease or disorder is a genetic disorder. In some embodiments, the genetic disorder is sickle cell anemia, Glycogen storage diseases (GSD, e.g., GSD types I, II, III, IV, V, VI, VII, VIII, IX, X, XI, XII, XIII, and XIV), cystic fibrosis, lysosomal acid lipase (LAL) deficiency 1, Tay-Sachs disease, Phenylketonuria, Mucopolysaccharidoses, Galactosemia, muscular dystrophy (e.g., Duchenne muscular dystrophy), hemophilia such as hemophilia A (classic hemophilia) or hemophilia B (Christmas Disease), Wilson's disease, Fabry Disease, Gaucher Disease hereditary angioedema (HAE), and alpha 1 antitrypsin deficiency. Examples of other diseases or disorders are provided above in Section 6.7.1.

In some aspects, the disease or condition is a disease of the CNS. Exemplary diseases of the CNS include, Absence of the Septum Pellucidum, Acid Lipase Disease, Acid Maltase Deficiency, Acquired Epileptiform Aphasia, Acute Disseminated Encephalomyelitis, Attention Deficit-Hyperactivity Disorder (ADHD), Adie's Pupil, Adie's Syndrome, Adrenoleukodystrophy, Agenesis of the Corpus Callosum, Agnosia, Aicardi Syndrome, Aicardi-Goutieres Syndrome Disorder, AIDS-Neurological Complications, Alexander Disease, Alpers' Disease, Alternating Hemiplegia, Alzheimer's Disease, Amyotrophic Lateral Sclerosis (ALS), Anencephaly, Aneurysm, Angelman Syndrome, Angiomatosis, Angleman syndrome, Anoxia, Antiphospholipid Syndrome, Aphasia, Apraxia, Arachnoid Cysts, Arachnoiditis, Arnold-Chiari Malformation, Arteriovenous Malformation, Asperger Syndrome, Ataxia, Ataxia Telangiectasia, Ataxias and Cerebellar or Spinocerebellar Degeneration, Atrial Fibrillation and Stroke, Attention Deficit-Hyperactivity Disorder, Autism Spectrum Disorder, Autonomic Dysfunction, Back Pain, Barth Syndrome, Batten Disease, Becker's Myotonia, Bechet's Disease, Bell's Palsy, Benign Essential Blepharospasm, Benign Focal Amyotrophy, Benign Intracranial Hypertension, Bernhardt-Roth Syndrome, Binswanger's Disease, Blepharospasm, Bloch-Sulzberger Syndrome, Brachial Plexus Birth Injuries, Brachial Plexus Injuries, Bradbury-Eggleston Syndrome, Brain and Spinal Tumors, Brain Aneurysm, Brain Injury, Brown-Sequard Syndrome, Bulbar palsy, Bulbospinal Muscular Atrophy, Cerebral Autosomal Dominant Arteriopathy with Sub-cortical Infarcts and Leukoencephalopathy (CADASIL), Canavan Disease, Carpal Tunnel Syndrome, Causalgia, Cavernomas, Cavernous Angioma, Cavernous Malformation, Central Cervical Cord Syndrome, Central Cord Syndrome, Central Pain Syndrome, Central Pontine Myelinolysis, Cephalic Disorders, Ceramidase Deficiency, Cerebellar Degeneration, Cerebellar Hypoplasia, Cerebral Aneurysms, Cerebral Arteriosclerosis, Cerebral Atrophy, Cerebral Beriberi, Cerebral Cavernous Malformation, Cerebral Gigantism, Cerebral Hypoxia, Cerebral Palsy, Cerebro-Oculo-Facio-Skeletal Syndrome (COFS), Charcot-Marie-Tooth Disease, Chiari Malformation, Cholesterol Ester Storage Disease, Chorea, Choreoacanthocytosis, Chronic Inflammatory Demyelinating Polyneuropathy (CIDP), Chronic Orthostatic Intolerance, Chronic Pain, Cockayne Syndrome Type II, Coffin Lowry Syndrome, Colpocephaly, Coma, Complex Regional Pain Syndrome, Concentric sclerosis (Balô's sclerosis), Congenital Facial Diplegia, Congenital Myasthenia, Congenital Myopathy, Congenital Vascular Cavernous Malformations, Corticobasal Degeneration, Cranial Arteritis, Craniosynostosis, Cree encephalitis, Creutzfeldt-Jakob Disease, Chronic progressive external ophtalmoplegia, Cumulative Trauma Disorders, Cushing's Syndrome, Cytomegalic Inclusion Body Disease, Cytomegalovirus Infection, Dancing Eyes-Dancing Feet Syndrome, Dandy-Walker Syndrome, Dawson Disease, De Morsier's Syndrome, Dejerine-Klumpke Palsy, Dementia, Dementia-Multi-Infarct, Dementia-Semantic, Dementia-Subcortical, Dementia With Lewy Bodies, Demyelination diseases, Dentate Cerebellar Ataxia, Dentatorubral Atrophy, Dermatomyositis, Developmental Dyspraxia, Devic's Syndrome, Diabetic Neuropathy, Diffuse Sclerosis, Distal hereditary motor neuronopathies, Dravet Syndrome, Dysautonomia, Dysgraphia, Dyslexia, Dysphagia, Dyspraxia, Dyssynergia Cerebellaris Myoclonica, Dyssynergia Cerebellaris Progressiva, Dystonias, Early Infantile Epileptic Encephalopathy, Empty Sella Syndrome, Encephalitis, Encephalitis Lethargica, Encephaloceles, Encephalomyelitis, Encephalopathy, Encephalopathy (familial infantile), Encephalotrigeminal Angiomatosis, Epilepsy, Epileptic Hemiplegia, Episodic ataxia, Erb's Palsy, Erb-Duchenne and Dejerine-Klumpke Palsies, Essential Tremor, Extrapontine Myelinolysis, Faber's disease, Fabry Disease, Fahr's Syndrome, Fainting, Familial Dysautonomia, Familial Hemangioma, Familial Idiopathic Basal Ganglia Calcification, Familial Periodic Paralyses, Familial Spastic Paralysis, Farber's Disease, Febrile Seizures, Fibromuscular Dysplasia, Fisher Syndrome, Floppy Infant Syndrome, Foot Drop, Fragile X syndrome, Friedreich's Ataxia, Frontotemporal Dementia, Gaucher Disease, Generalized Gangliosidoses (GM1, GM2), Gerstmann's Syndrome, Gerstmann-Straussler-Scheinker Disease, Giant Axonal Neuropathy, Giant Cell Arteritis, Giant Cell Inclusion Disease, Globoid Cell Leukodystrophy, Glossopharyngeal Neuralgia, Glycogen Storage Disease, Guillain-Barré Syndrome, Hallervorden-Spatz Disease, Head Injury, Headache, Hemicrania Continua, Hemifacial Spasm, Hemiplegia Alterans, Hereditary Neuropathies, Hereditary Spastic Paraplegia, Heredopathia Atactica Polyneuritiformis, Herpes Zoster, Herpes Zoster Oticus, Hirayama Syndrome, Holmes-Adie syndrome, Holoprosencephaly, HTLV-1 Associated Myelopathy, Hughes Syndrome, Huntington's Disease, Hurler syndrome, Hydranencephaly, Hydrocephalus, Hydrocephalus—Normal Pressure, Hydromyelia, Hypercortisolism, Hypersomnia, Hypertonia, Hypotonia, Hypoxia, Immune-Mediated Encephalomyelitis, Inclusion Body Myositis, Incontinentia Pigmenti, Infantile Hypotonia, Infantile Neuroaxonal Dystrophy, Infantile Phytanic Acid Storage Disease, Infantile Refsum Disease, Infantile Spasms, Inflammatory Myopathies, Iniencephaly, Intestinal Lipodystrophy, Intracranial Cysts, Intracranial Hypertension, Isaacs' Syndrome, Joubert Syndrome, Kearns-Sayre Syndrome, Kennedy's Disease, Kinsbourne syndrome, Kleine-Levin Syndrome, Klippel-Feil Syndrome, Klippel-Trenaunay Syndrome (KTS), Klüver-Bucy Syndrome, Korsakoff's Amnesic Syndrome, Krabbe Disease, Kugelberg-Welander Disease, Kuru, Lambert-Eaton Myasthenic Syndrome, Landau-Kleffner Syndrome, Lateral Femoral Cutaneous Nerve Entrapment, Lateral Medullary Syndrome, Learning Disabilities, Leigh's Disease, Lennox-Gastaut Syndrome, Lesch-Nyhan Syndrome, Leukodystrophy, Levine-Critchley Syndrome, Lewy Body Dementia, Lichtheim's disease, Lipid Storage Diseases, Lipoid Proteinosis, Lissencephaly, Locked-In Syndrome, Lou Gehrig's Disease, Lupus—Neurological Sequelae, Lyme Disease—Neurological Complications, Lysosomal storage disorders, Machado-Joseph Disease, Macrencephaly, Megalencephaly, Melkersson-Rosenthal Syndrome, Meningitis, Meningitis and Encephalitis, Menkes Disease, Meralgia Paresthetica, Metachromatic Leukodystrophy, Microcephaly, Migraine, Miller Fisher Syndrome, Mini Stroke, Mitochondrial Myopathy, Mitochondrial DNAdepletion syndromes, Moebius Syndrome, Monomelic Amyotrophy, Morvan Syndrome, Motor Neuron Diseases, Moyamoya Disease, Mucolipidoses, Mucopolysaccharidoses, Multi-Infarct Dementia, Multifocal Motor Neuropathy, Multiple Sclerosis, Multiple System Atrophy, Multiple System Atrophy with Orthostatic Hypotension, Muscular Dystrophy, Myasthenia—Congenital, Myasthenia Gravis, Myelinoclastic Diffuse Sclerosis, Myelitis, Myoclonic Encephalopathy of Infants, Myoclonus, Myoclonus epilepsy, Myopathy, Myopathy-Congenital, Myopathy-Thyrotoxic, Myotonia, Myotonia Congenita, Narcolepsy, NARP (neuropathy, ataxia and retinitis pigmentosa), Neuroacanthocytosis, Neurodegeneration with Brain Iron Accumulation, Neurodegenerative disease, Neurofibromatosis, Neuroleptic Malignant Syndrome, Neurological Complications of AIDS, Neurological Complications of Lyme Disease, Neurological Consequences of Cytomegalovirus Infection, Neurological Manifestations of Pompe Disease, Neurological Sequelae Of Lupus, Neuromyelitis Optica, Neuromyotonia, Neuronal Ceroid Lipofuscinosis, Neuronal Migration Disorders, Neuropathic pain, Neuropathy-Hereditary, Neuropathy, Neurosarcoidosis, Neurosyphilis, Neurotoxicity, Nevus Cavernosus, Niemann-Pick Disease, O'Sullivan-McLeod Syndrome, Occipital Neuralgia, Ohtahara Syndrome, Olivopontocerebellar Atrophy, Opsoclonus Myoclonus, Orthostatic Hypotension, Overuse Syndrome, Pain-Chronic, Pantothenate Kinase-Associated Neurodegeneration, Paraneoplastic Syndromes, Paresthesia, Parkinson's Disease, Paroxysmal Choreoathetosis, Paroxysmal Hemicrania, Parry-Romberg, Pelizaeus-Merzbacher Disease, Pena Shokeir II Syndrome, Perineural Cysts, Peroneal muscular atrophy, Periodic Paralyses, Peripheral Neuropathy, Periventricular Leukomalacia, Persistent Vegetative State, Pervasive Developmental Disorders, Phytanic Acid Storage Disease, Pick's Disease, Pinched Nerve, Piriformis Syndrome, Pituitary Tumors, Polymyositis, Pompe Disease, Porencephaly, Post-Polio Syndrome, Postherpetic Neuralgia, Postinfectious Encephalomyelitis, Postural Hypotension, Postural Orthostatic Tachycardia Syndrome, Postural Tachycardia Syndrome, Primary Dentatum Atrophy, Primary Lateral Sclerosis, Primary Progressive Aphasia, Prion Diseases, Progressive bulbar palsy, Progressive Hemifacial Atrophy, Progressive Locomotor Ataxia, Progressive Multifocal Leukoencephalopathy, Progressive Muscular Atrophy, Progressive Sclerosing Poliodystrophy, Progressive Supranuclear Palsy, Prosopagnosia, Pseudobulbar palsy, Pseudo-Torch syndrome, Pseudotoxoplasmosis syndrome, Pseudotumor Cerebri, Psychogenic Movement, Ramsay Hunt Syndrome I, Ramsay Hunt Syndrome II, Rasmussen's Encephalitis, Reflex Sympathetic Dystrophy Syndrome, Refsum Disease, Refsum Disease—Infantile, Repetitive Motion Disorders, Repetitive Stress Injuries, Restless Legs Syndrome, Retrovirus-Associated Myelopathy, Rett Syndrome, Reye's Syndrome, Rheumatic Encephalitis, Riley-Day Syndrome, Sacral Nerve Root Cysts, Saint Vitus Dance, Salivary Gland Disease, Sandhoff Disease, Schilder's Disease, Schizencephaly, Seitelberger Disease, Seizure Disorder, Semantic Dementia, Septo-Optic Dysplasia, Severe Myoclonic Epilepsy of Infancy (SMEI), Shaken Baby Syndrome, Shingles, Shy-Drager Syndrome, Sjögren's Syndrome, Sleep Apnea, Sleeping Sickness, Sotos Syndrome, Spasticity, Spina Bifida, Spinal Cord Infarction, Spinal Cord Injury, Spinal Cord Tumors, Spinal Muscular Atrophy, Spinocerebellar Ataxia, Spinocerebellar Atrophy, Spinocerebellar Degeneration, Sporadic ataxia, Steele-Richardson-Olszewski Syndrome, Stiff-Person Syndrome, Striatonigral Degeneration, Stroke, Sturge-Weber Syndrome, Subacute Sclerosing Panencephalitis, Subcortical Arteriosclerotic Encephalopathy, Short-lasting, Unilateral, Neuralgiform (SUNCT) Headache, Swallowing Disorders, Sydenham Chorea, Syncope, Syphilitic Spinal Sclerosis, Syringohydromyelia, Syringomyelia, Systemic Lupus Erythematosus, Tabes Dorsalis, Tardive Dyskinesia, Tarlov Cysts, Tay-Sachs Disease, Temporal Arteritis, Tethered Spinal Cord Syndrome, Thomsen's Myotonia, Thoracic Outlet Syndrome, Thyrotoxic Myopathy, Tic Douloureux, Todd's Paralysis, Tourette Syndrome, Transient Ischemic Attack, Transmissible Spongiform Encephalopathies, Transverse Myelitis, Traumatic Brain Injury, Tremor, Trigeminal Neuralgia, Tropical Spastic Paraparesis, Troyer Syndrome, Tuberous Sclerosis, Vascular Erectile Tumor, Vasculitis Syndromes of the Central and Peripheral Nervous Systems, Vitamin B12 deficiency, Von Economo's Disease, Von Hippel-Lindau Disease (VHL), Von Recklinghausen's Disease, Wallenberg's Syndrome, Werdnig-Hoffman Disease, Wernicke-Korsakoff Syndrome, West Syndrome, Whiplash, Whipple's Disease, Williams Syndrome, Wilson Disease, Wolman's Disease, X-Linked Spinal and Bulbar Muscular Atrophy. Examples of other diseases or disorders are provided above in Section 6.7.1.

In some aspects, the disease or condition is a disease of muscle. Exemplary diseases of the muscle include Acid Maltase Deficiency (AMD), Amyotrophic Lateral Sclerosis (ALS), Andersen-Tawil Syndrome, Becker Muscular Dystrophy (BMD), Becker Myotonia Congenita, Bethlem Myopathy, Bulbospinal Muscular Atrophy (Spinal-Bulbar Muscular Atrophy), Carnitine Deficiency, Carnitine Palmityl Transferase Deficiency (CPT Deficiency), Central Core Disease (CCD), Centronuclear Myopathy, Charcot-Marie-Tooth Disease (CMT), Congenital Muscular Dystrophy (CMD), Congenital Myasthenic Syndromes (CMS), Congenital Myotonic Dystrophy, Cori Disease (Debrancher Enzyme Deficiency), Debrancher Enzyme Deficiency, Dejerine-Sottas Disease (DSD), Dermatomyositis (DM), Distal Muscular Dystrophy (DD), Duchenne Muscular Dystrophy (DMD), Dystrophia Myotonica (Myotonic Muscular Dystrophy), Emery-Dreifuss Muscular Dystrophy (EDMD), Endocrine Myopathies, Eulenberg Disease (Paramyotonia Congenita), Facioscapulohumeral Muscular Dystrophy (FSH or FSHD), Finnish (Tibial) Distal Myopathy, Forbes Disease (Debrancher Enzyme Deficiency), Friedreich's Ataxia (FA), Fukuyama Congenital Muscular Dystrophy, Glycogenosis Type 10, Glycogenosis Type 11, Glycogenosis Type 2, Glycogenosis Type 3, Glycogenosis Type 5, Glycogenosis Type 7, Glycogenosis Type 9, Gowers-Laing Distal Myopathy, Hauptmann-Thanheuser MD (Emery-Dreifuss Muscular Dystrophy), Hereditary Inclusion-Body Myositis, Hereditary Motor and Sensory Neuropathy (Charcot-Marie-Tooth Disease), Hyperthyroid Myopathy, Hypothyroid Myopathy, Inclusion-Body Myositis (IBM), Inherited Myopathies, Integrin-Deficient Congenital Muscular Dystrophy, Kennedy Disease (Spinal-Bulbar Muscular Atrophy), Kugelberg-Welander Disease (Spinal Muscular Atrophy), Lactate Dehydrogenase Deficiency, Lambert-Eaton Myasthenic Syndrome (LEMS), Limb-Girdle Muscular Dystrophy (LGMD), Lou Gehrig's Disease (Amyotrophic Lateral Sclerosis), McArdle Disease (Phosphorylase Deficiency), Merosin-Deficient Congenital Muscular Dystrophy, Metabolic Diseases of Muscle, Mitochondrial Myopathy, Miyoshi Distal Myopathy, Motor Neurone Disease, Muscle-Eye-Brain Disease, Myasthenia Gravis (MG), Myoadenylate Deaminase Deficiency, Myofibrillar Myopathy, Myophosphorylase Deficiency, Myotonia Congenita (MC), Myotonic Muscular Dystrophy (MMD), Myotubular Myopathy (MTM or MM), Nemaline Myopathy, Nonaka Distal Myopathy, Oculopharyngeal Muscular Dystrophy (OPMD), Paramyotonia Congenita, Pearson Syndrome, Periodic Paralysis, Peroneal Muscular Atrophy (Charcot-Marie-Tooth Disease), Phosphofructokinase Deficiency, Phosphoglycerate Kinase Deficiency, Phosphoglycerate Mutase Deficiency, Phosphorylase Deficiency, Phosphorylase Deficiency, Polymyositis (PM), Pompe Disease (Acid Maltase Deficiency), Progressive External Ophthalmoplegia (PEO), Rod Body Disease (Nemaline Myopathy), Spinal Muscular Atrophy (SMA), Spinal-Bulbar Muscular Atrophy (SBMA), Steinert Disease (Myotonic Muscular Dystrophy), Tarui Disease (Phosphofructokinase Deficiency), Thomsen Disease (Myotonia Congenita), Ullrich Congenital Muscular Dystrophy, Walker-Warburg Syndrome (Congenital Muscular Dystrophy), Welander Distal Myopathy, Werdnig-Hoffmann Disease (Spinal Muscular Atrophy), and ZASP-Related Myopathy. Additional examples of muscle diseases and disorders are provided above in Section 6.7.1.

In some aspects, the disease or condition is a disease affecting the CNS and muscle, for example SMA, multiple sclerosis, Amyotrophic lateral sclerosis (ALS), Ataxia, Becker muscular dystrophy, Charcot-Marie-Tooth disease, Dystonias, Friedreich's ataxia, Glycogen storage disease II, Kennedy Disease, Lambert-Eaton Myasthenic Syndrome, Mitochondrial DNA depletion syndromes, Muscle-Eye-Brain Disease, Neuromyotonia, Periodic Paralyses, juvenile Primary Lateral Sclerosis, Progressive external ophtalmoplegia, Spastic Paraplegias, congenital Stiff-Person Syndrome, Tardive Dyskinesia, Werdnig-Hoffman Disease, or X-Linked Spinal and Bulbar Muscular Atrophy.

In some embodiments, administration of a dependoparvovirus particle comprising a variant polypeptide and comprising a payload (e.g., a transgene as described in Section 6.7.1) to a subject induces expression of the payload (e.g., transgene) in a subject. In some embodiments, the expression is induced in muscle tissue. In some embodiments, the production is similar in the muscle tissue as compared to a similar particle with the wild-type capsid protein. In some embodiments, the production is increased in the muscle tissue as compared to a similar particle with the wild-type capsid protein. In some embodiments, the production is similar in the CNS as compared to a similar particle with the wild-type capsid protein. In some embodiments, the production is increased in the CNS as compared to a similar particle with the wild-type capsid protein. The amount of a payload, e.g., transgene, e.g., heterologous protein, e.g., therapeutic polypeptide, expressed in a subject (e.g., the serum of the subject) can vary. For example, in some embodiments the payload, e.g., protein or RNA product of a transgene, can be expressed in the serum of the subject in the amount of at least about 9 μg/ml, at least about 10 μg/ml, at least about 50 μg/ml, at least about 100 μg/ml, at least about 200 μg/ml, at least about 300 μg/ml, at least about 400 μg/ml, at least about 500 μg/ml, at least about 600 μg/ml, at least about 700 μg/ml, at least about 800 μg/ml, at least about 900 μg/ml, or at least about 1000 μg/ml. In some embodiments, the payload, e.g., protein or RNA product of a transgene, is expressed in the serum of the subject in the amount of about 9 μg/ml, about 10 μg/ml, about 50 μg/ml, about 100 μg/ml, about 200 μg/ml, about 300 μg/ml, about 400 μg/ml, about 500 μg/ml, about 600 μg/ml, about 700 μg/ml, about 800 μg/ml, about 900 μg/ml, about 1000 μg/ml, about 1500 μg/ml, about 2000 μg/ml, about 2500 μg/ml, or a range between any two of these values.

In some embodiments, for therapeutic applications, a viral particle comprising a capsid polypeptide as described herein is prepared as a pharmaceutical composition. As used herein the term “pharmaceutical composition” refers to a composition comprising at least one active ingredient (e.g., the viral particle) and optionally, one or more pharmaceutically acceptable carriers or excipients.

Relative amounts of the active ingredient, pharmaceutically acceptable carrier or excipient, and/or any additional ingredients in a pharmaceutical composition in accordance with the present disclosure may vary. Differences in the constitution of a pharmaceutical composition may depend upon the identity, size, and/or condition of the subject being treated, the route by which the composition is to be administered, and/or any other factor. The composition may comprise between 0.0001% and 99% (w/w) of the active ingredient. By way of example, the composition may comprise between 0.0001% and 100%, e.g., between 0.5 and 50%, between 1-30%, between 5-80%, or at least 80% (w/w) active ingredient. Non limiting examples of carriers and/or excipients include solvents, dispersion media, diluents, or other liquid vehicles, dispersion or suspension aids, surface active agents, isotonic agents, thickening or emulsifying agents, preservatives, or combination thereof.

### 7. Numbered Embodiments

While various specific embodiments have been illustrated and described, it will be appreciated that various changes can be made without departing from the spirit and scope of the disclosure(s). The present disclosure is exemplified by the numbered embodiments set forth below. Unless otherwise specified, features of any of the concepts, aspects and/or embodiments described in the detailed description above are applicable mutatis mutandis to any of the following numbered embodiments.
1. A capsid polypeptide comprising:
   (a) a histidine at a position corresponding to R550 of the VP1 capsid polypeptide of SEQ ID NO:7;
   (b) an asparagine at a position corresponding to D551 of the VP1 capsid polypeptide of SEQ ID NO:7;
   (c) an alanine at a position corresponding to S576 of the VP1 capsid polypeptide of SEQ ID NO:7;
   (d) an isoleucine at a position corresponding to Q579 of the VP1 capsid polypeptide of SEQ ID NO:7;
   (e) a lysine at a position corresponding to H584 of the VP1 capsid polypeptide of SEQ ID NO:7;
   (f) an asparagine at a position corresponding to S586 of the VP1 capsid polypeptide of SEQ ID NO:7;
   (g) a histidine or serine at a position corresponding to A587 of the VP1 capsid polypeptide of SEQ ID NO:7;
   (h) a tyrosine, a threonine, or asparagine at a position corresponding to Q588 of the VP1 capsid polypeptide of SEQ ID NO:7;
   (i) a glutamine at a position corresponding to A589 of the VP1 capsid polypeptide of SEQ ID NO:7;
   (j) an asparagine at a position corresponding to Q592 of the VP1 capsid polypeptide of SEQ ID NO:7; or
   (k) any combination of two, three, four, five, six, seven, eight, nine, or all ten of (a), (b), (c), (d), (e), (f), (g), (h), (i), and (j).
2. In a capsid polypeptide, the improvement comprising:
   (a) a histidine at a position corresponding to R550 of the VP1 capsid polypeptide of SEQ ID NO:7;
   (b) an asparagine at a position corresponding to D551 of the VP1 capsid polypeptide of SEQ ID NO:7;
   (c) an alanine at a position corresponding to S576 of the VP1 capsid polypeptide of SEQ ID NO:7;
   (d) an isoleucine at a position corresponding to Q579 of the VP1 capsid polypeptide of SEQ ID NO:7;
   (e) a lysine at a position corresponding to H584 of the VP1 capsid polypeptide of SEQ ID NO:7;
   (f) an asparagine at a position corresponding to S586 of the VP1 capsid polypeptide of SEQ ID NO:7;
   (g) a histidine or serine at a position corresponding to A587 of the VP1 capsid polypeptide of SEQ ID NO:7;
   (h) a tyrosine, a threonine, or asparagine at a position corresponding to Q588 of the VP1 capsid polypeptide of SEQ ID NO:7;
   (i) a glutamine at a position corresponding to A589 of the VP1 capsid polypeptide of SEQ ID NO:7;
   (j) an asparagine at a position corresponding to Q592 of the VP1 capsid polypeptide of SEQ ID NO:7; or
   (k) any combination of two, three, four, five, six, seven, eight, nine, or all ten of (a), (b), (c), (d), (e), (f), (g), (h), (i), and (j).
3. The capsid polypeptide of embodiment 1 or embodiment 2, which comprises (i) a histidine at a position corresponding to R550 of the VP1 capsid polypeptide of SEQ ID NO:7, (ii) an asparagine at a position corresponding to D551 of the VP1 capsid polypeptide of SEQ ID NO:7, (iii) an alanine at a position corresponding to S576 of the VP1 capsid polypeptide of SEQ ID NO:7, (iv) a lysine at a position corresponding to H584 of the VP1 capsid polypeptide of SEQ ID NO:7, (v) a tyrosine at a position corresponding to Q588 of the VP1 capsid polypeptide of SEQ ID NO:7, or (vi) any combination of two, three, four, or all five of (i)-(v).
4. The capsid polypeptide of embodiment 3, which comprises (i) a histidine at a position corresponding to R550 of the VP1 capsid polypeptide of SEQ ID NO:7, (ii) an alanine at a position corresponding to S576 of the VP1 capsid polypeptide of SEQ ID NO:7, (iii) a lysine at a position corresponding to H584 of the VP1 capsid polypeptide of SEQ ID NO:7, (iv) a tyrosine at a position corresponding to Q588 of the VP1 capsid polypeptide of SEQ ID NO:7, or (v) any combination of two, three, or all four of (i)-(iv).
5. The capsid polypeptide of any one of embodiments 1 to 4, which comprises a histidine at a position corresponding to R550 of the VP1 capsid polypeptide of SEQ ID NO:7.
6. The capsid polypeptide of any one of embodiments 1 to 5, which comprises an asparagine at a position corresponding to D551 of the VP1 capsid polypeptide of SEQ ID NO:7.
7. The capsid polypeptide of any one of embodiments 1 to 6, which comprises an alanine at a position corresponding to S576 of the VP1 capsid polypeptide of SEQ ID NO:7.
8. The capsid polypeptide of any one of embodiments 1 to 7, which comprises an isoleucine at a position corresponding to Q579 of the VP1 capsid polypeptide of SEQ ID NO:7.
9. The capsid polypeptide of any one of embodiments 1 to 7, which comprises a threonine at a position corresponding to Q579 of the VP1 capsid polypeptide of SEQ ID NO:7.
10. The capsid polypeptide of any one of embodiments 1 to 9, which comprises a lysine at a position corresponding to H584 of the VP1 capsid polypeptide of SEQ ID NO:7.
11. The capsid polypeptide of any one of embodiments 1 to 9, which comprises an asparagine at a position corresponding to S586 of the VP1 capsid polypeptide of SEQ ID NO:7.
12. The capsid polypeptide of any one of embodiments 1 to 11, which comprises a histidine at a position corresponding to A587 of the VP1 capsid polypeptide of SEQ ID NO:7.
13. The capsid polypeptide of any one of embodiments 1 to 11, which comprises a serine at a position corresponding to A587 of the VP1 capsid polypeptide of SEQ ID NO:7.
14. The capsid polypeptide of any one of embodiments 1 to 13, which comprises a tyrosine at a position corresponding to Q588 of the VP1 capsid polypeptide of SEQ ID NO:7.
15. The capsid polypeptide of any one of embodiments 1 to 13, which comprises a threonine at a position corresponding to Q588 of the VP1 capsid polypeptide of SEQ ID NO:7.
16. The capsid polypeptide of any one of embodiments 1 to 13, which comprises an asparagine at a position corresponding to Q588 of the VP1 capsid polypeptide of SEQ ID NO:7.
17. The capsid polypeptide of any one of embodiments 1 to 16, which comprises a glutamine at a position corresponding to A589 of the VP1 capsid polypeptide of SEQ ID NO:7.
18. The capsid polypeptide of any one of embodiments 1 to 16, which comprises a threonine at a position corresponding to A589 of the VP1 capsid polypeptide of SEQ ID NO:7.
19. The capsid polypeptide of any one of embodiments 1 to 18, which comprises an asparagine at a position corresponding to Q592 of the VP1 capsid polypeptide of SEQ ID NO:7.
20. The capsid polypeptide of any one of embodiments 1 to 19, which comprises a valine at a position corresponding to T593 of the VP1 capsid polypeptide of SEQ ID NO:7.
21. The capsid polypeptide of any one of embodiments 1 to 3, which comprises a histidine at a position corresponding to R550 of the VP1 capsid polypeptide of SEQ ID NO:7 and an asparagine at a position corresponding to D551 of the VP1 capsid polypeptide of SEQ ID NO:7.
22. The capsid polypeptide of any one of embodiments 1 to 3, which comprises a histidine at a position corresponding to R550 of the VP1 capsid polypeptide of SEQ ID NO:7 and an alanine at a position corresponding to S576 of the VP1 capsid polypeptide of SEQ ID NO:7.
23. The capsid polypeptide of any one of embodiments 1 to 3, which comprises a histidine at a position corresponding to R550 of the VP1 capsid polypeptide of SEQ ID NO:7 and a lysine at a position corresponding to H584 of the VP1 capsid polypeptide of SEQ ID NO:7.
24. The capsid polypeptide of any one of embodiments 1 to 3, which comprises a histidine at a position corresponding to R550 of the VP1 capsid polypeptide of SEQ ID NO:7 and a tyrosine at a position corresponding to Q588 of the VP1 capsid polypeptide of SEQ ID NO:7.
25. The capsid polypeptide of any one of embodiments 1 to 3, which comprises an asparagine at a position corresponding to D551 of the VP1 capsid polypeptide of SEQ ID NO:7 and an alanine at a position corresponding to S576 of the VP1 capsid polypeptide of SEQ ID NO:7.
26. The capsid polypeptide of any one of embodiments 1 to 3, which comprises an asparagine at a position corresponding to D551 of the VP1 capsid polypeptide of SEQ ID NO:7 and a lysine at a position corresponding to H584 of the VP1 capsid polypeptide of SEQ ID NO:7.
27. The capsid polypeptide of any one of embodiments 1 to 3, which comprises an asparagine at a position corresponding to D551 of the VP1 capsid polypeptide of SEQ ID NO:7 and a tyrosine at a position corresponding to Q588 of the VP1 capsid polypeptide of SEQ ID NO:7.
28. The capsid polypeptide of any one of embodiments 1 to 3, which comprises an alanine at a position corresponding to S576 of the VP1 capsid polypeptide of SEQ ID NO:7 and a lysine at a position corresponding to H584 of the VP1 capsid polypeptide of SEQ ID NO:7.
29. The capsid polypeptide of any one of embodiments 1 to 3, which comprises an alanine at a position corresponding to S576 of the VP1 capsid polypeptide of SEQ ID NO:7 and a tyrosine at a position corresponding to Q588 of the VP1 capsid polypeptide of SEQ ID NO:7.
30. The capsid polypeptide of any one of embodiments 1 to 3, which comprises a lysine at a position corresponding to H584 of the VP1 capsid polypeptide of SEQ ID NO:7 and a tyrosine at a position corresponding to Q588 of the VP1 capsid polypeptide of SEQ ID NO:7.
31. The capsid polypeptide of any one of embodiments 1 to 3, which comprises a histidine at a position corresponding to R550 of the VP1 capsid polypeptide of SEQ ID NO:7, an asparagine at a position corresponding to D551 of the VP1 capsid polypeptide of SEQ ID NO:7, and an alanine at a position corresponding to S576 of the VP1 capsid polypeptide of SEQ ID NO:7.
32. The capsid polypeptide of any one of embodiments 1 to 3, which comprises a histidine at a position corresponding to R550 of the VP1 capsid polypeptide of SEQ ID NO:7, an asparagine at a position corresponding to D551 of the VP1 capsid polypeptide of SEQ ID NO:7, and a lysine at a position corresponding to H584 of the VP1 capsid polypeptide of SEQ ID NO:7.
33. The capsid polypeptide of any one of embodiments 1 to 3, which comprises a histidine at a position corresponding to R550 of the VP1 capsid polypeptide of SEQ ID NO:7, an asparagine at a position corresponding to D551 of the VP1 capsid polypeptide of SEQ ID NO:7, and a tyrosine at a position corresponding to Q588 of the VP1 capsid polypeptide of SEQ ID NO:7.
34. The capsid polypeptide of any one of embodiments 1 to 3, which comprises a histidine at a position corresponding to R550 of the VP1 capsid polypeptide of SEQ ID NO:7, an alanine at a position corresponding to S576 of the VP1 capsid polypeptide of SEQ ID NO:7, and a lysine at a position corresponding to H584 of the VP1 capsid polypeptide of SEQ ID NO:7.
35. The capsid polypeptide of any one of embodiments 1 to 3, which comprises a histidine at a position corresponding to R550 of the VP1 capsid polypeptide of SEQ ID NO:7, an alanine at a position corresponding to S576 of the VP1 capsid polypeptide of SEQ ID NO:7, and a tyrosine at a position corresponding to Q588 of the VP1 capsid polypeptide of SEQ ID NO:7.
36. The capsid polypeptide of any one of embodiments 1 to 3, which comprises a histidine at a position corresponding to R550 of the VP1 capsid polypeptide of SEQ ID NO:7, a lysine at a position corresponding to H584 of the VP1 capsid polypeptide of SEQ ID NO:7, and a tyrosine at a position corresponding to Q588 of the VP1 capsid polypeptide of SEQ ID NO:7.
37. The capsid polypeptide of any one of embodiments 1 to 3, which comprises an asparagine at a position corresponding to D551 of the VP1 capsid polypeptide of SEQ ID NO:7, an alanine at a position corresponding to S576 of the VP1 capsid polypeptide of SEQ ID NO:7, and a lysine at a position corresponding to H584 of the VP1 capsid polypeptide of SEQ ID NO:7.
38. The capsid polypeptide of any one of embodiments 1 to 3, which comprises an asparagine at a position corresponding to D551 of the VP1 capsid polypeptide of SEQ ID NO:7, an alanine at a position corresponding to S576 of the VP1 capsid polypeptide of SEQ ID NO:7, and a tyrosine at a position corresponding to Q588 of the VP1 capsid polypeptide of SEQ ID NO:7.
39. The capsid polypeptide of any one of embodiments 1 to 3, which comprises an asparagine at a position corresponding to D551 of the VP1 capsid polypeptide of SEQ ID NO:7, a lysine at a position corresponding to H584 of the VP1 capsid polypeptide of SEQ ID NO:7, and a tyrosine at a position corresponding to Q588 of the VP1 capsid polypeptide of SEQ ID NO:7.
40. The capsid polypeptide of any one of embodiments 1 to 3, which comprises an alanine at a position corresponding to S576 of the VP1 capsid polypeptide of SEQ ID NO:7, a lysine at a position corresponding to H584 of the VP1 capsid polypeptide of SEQ ID NO:7, and a tyrosine at a position corresponding to Q588 of the VP1 capsid polypeptide of SEQ ID NO:7.
41. The capsid polypeptide of any one of embodiments 1 to 3, which comprises a histidine at a position corresponding to R550 of the VP1 capsid polypeptide of SEQ ID NO:7, an asparagine at a position corresponding to D551 of the VP1 capsid polypeptide of SEQ ID NO:7, an alanine at a position corresponding to S576 of the VP1 capsid polypeptide of SEQ ID NO:7, and a lysine at a position corresponding to H584 of the VP1 capsid polypeptide of SEQ ID NO:7.
42. The capsid polypeptide of any one of embodiments 1 to 3, which comprises a histidine at a position corresponding to R550 of the VP1 capsid polypeptide of SEQ ID NO:7, an asparagine at a position corresponding to D551 of the VP1 capsid polypeptide of SEQ ID NO:7, an alanine at a position corresponding to S576 of the VP1 capsid polypeptide of SEQ ID NO:7, and a tyrosine at a position corresponding to Q588 of the VP1 capsid polypeptide of SEQ ID NO:7.
43. The capsid polypeptide of any one of embodiments 1 to 3, which comprises a histidine at a position corresponding to R550 of the VP1 capsid polypeptide of SEQ ID NO:7, an asparagine at a position corresponding to D551 of the VP1 capsid polypeptide of SEQ ID NO:7, a lysine at a position corresponding to H584 of the VP1 capsid polypeptide of SEQ ID NO:7, and a tyrosine at a position corresponding to Q588 of the VP1 capsid polypeptide of SEQ ID NO:7.
44. The capsid polypeptide of any one of embodiments 1 to 3, which comprises a histidine at a position corresponding to R550 of the VP1 capsid polypeptide of SEQ ID NO:7, an alanine at a position corresponding to S576 of the VP1 capsid polypeptide of SEQ ID NO:7, a lysine at a position corresponding to H584 of the VP1 capsid polypeptide of SEQ ID NO:7, and a tyrosine at a position corresponding to Q588 of the VP1 capsid polypeptide of SEQ ID NO:7.
45. The capsid polypeptide of any one of embodiments 1 to 3, which comprises an asparagine at a position corresponding to D551 of the VP1 capsid polypeptide of SEQ ID NO:7, an alanine at a position corresponding to S576 of the VP1 capsid polypeptide of SEQ ID NO:7, a lysine at a position corresponding to H584 of the VP1 capsid polypeptide of SEQ ID NO:7, and a tyrosine at a position corresponding to Q588 of the VP1 capsid polypeptide of SEQ ID NO:7.
46. The capsid polypeptide of any one of embodiments 1 to 3, which comprises a histidine at a position corresponding to R550 of the VP1 capsid polypeptide of SEQ ID NO:7, an asparagine at a position corresponding to D551 of the VP1 capsid polypeptide of SEQ ID NO:7, an alanine at a position corresponding to S576 of the VP1 capsid polypeptide of SEQ ID NO:7, a lysine at a position corresponding to H584 of the VP1 capsid polypeptide of SEQ ID NO:7, and a tyrosine at a position corresponding to Q588 of the VP1 capsid polypeptide of SEQ ID NO:7.
47. The capsid polypeptide of any one of embodiments 21 to 46, further comprising an isoleucine at a position corresponding to Q579 of the VP1 capsid polypeptide of SEQ ID NO:7.
48. The capsid polypeptide of any one of embodiments 1 to 47, further comprising an alanine at a position corresponding to W595 of the VP1 capsid polypeptide of SEQ ID NO:7.
49. The capsid polypeptide of any one of embodiments 1 to 48, further comprising a leucine at a position corresponding to V596 of the VP1 capsid polypeptide of SEQ ID NO:7.
50. The capsid polypeptide of any one of embodiments 1 to 49, further comprising a serine at a position corresponding to N598 of the VP1 capsid polypeptide of SEQ ID NO:7.
51. The capsid polypeptide of any one of embodiments 1 to 50, further comprising a valine at a position corresponding to 1601 of the VP1 capsid polypeptide of SEQ ID NO:7.
52. The capsid polypeptide of any one of embodiments 1 to 47, further comprising an alanine at a position corresponding to W595 of the VP1 capsid polypeptide of SEQ ID NO:7, a leucine at a position corresponding to V596 of the VP1 capsid polypeptide of SEQ ID NO:7, and a serine at a position corresponding to N598 of the VP1 capsid polypeptide of SEQ ID NO:7.
53. The capsid polypeptide of any one of embodiments 1 to 47, further comprising an alanine at a position corresponding to W595 of the VP1 capsid polypeptide of SEQ ID NO:7, a leucine at a position corresponding to V596 of the VP1 capsid polypeptide of SEQ ID NO:7, a serine at a position corresponding to N598 of the VP1 capsid polypeptide of SEQ ID NO:7, and a valine at a position corresponding to 1601 of the VP1 capsid polypeptide of SEQ ID NO:7.
54. The capsid polypeptide of embodiment 1 or embodiment 2, which comprises a lysine at a position corresponding to H584 of the VP1 capsid polypeptide of SEQ ID NO:7 and an alanine at a position corresponding to W595 of the VP1 capsid polypeptide of SEQ ID NO:7.
55. The capsid polypeptide of embodiment 1 or embodiment 2, which comprises a tyrosine at a position corresponding to Q588 of the VP1 capsid polypeptide of SEQ ID NO:7 and an alanine at a position corresponding to W595 of the VP1 capsid polypeptide of SEQ ID NO:7.
56. The capsid polypeptide of embodiment 1 or embodiment 2, which comprises a lysine at a position corresponding to H584 of the VP1 capsid polypeptide of SEQ ID NO:7, a tyrosine at a position corresponding to Q588 of the VP1 capsid polypeptide of SEQ ID NO:7, and an alanine at a position corresponding to W595 of the VP1 capsid polypeptide of SEQ ID NO:7.
57. The capsid polypeptide of any one of embodiments 54 to 56, which comprises (i) a histidine at a position corresponding to R550 of the VP1 capsid polypeptide of SEQ ID NO:7, (ii) an alanine at a position corresponding to S576 of the VP1 capsid polypeptide of SEQ ID NO:7, (iii) a threonine at a position corresponding to Q579 of the VP1 capsid polypeptide of SEQ ID NO:7, (iv) a leucine at a position corresponding to V596 of the VP1 capsid polypeptide of SEQ ID NO:7, (v) a serine at a position corresponding to N598 of the VP1 capsid polypeptide of SEQ ID NO:7, (vi) a valine at a position corresponding to 1601 of the VP1 capsid polypeptide of SEQ ID NO:7, or (vii) any combination of two, three, four, five, or all six of (i)-(iv).
58. The capsid polypeptide of any one of embodiments 54 to 57, which comprises a histidine at a position corresponding to R550 of the VP1 capsid polypeptide of SEQ ID NO:7.
59. The capsid polypeptide of any one of embodiments 54 to 58, which comprises an alanine at a position corresponding to S576 of the VP1 capsid polypeptide of SEQ ID NO:7.
60. The capsid polypeptide of any one of embodiments 54 to 59, which comprises a threonine at a position corresponding to Q579 of the VP1 capsid polypeptide of SEQ ID NO:7.
61. The capsid polypeptide of any one of embodiments 54 to 60, which comprises a leucine at a position corresponding to V596 of the VP1 capsid polypeptide of SEQ ID NO:7.
62. The capsid polypeptide of any one of embodiments 54 to 61, which comprises a serine at a position corresponding to N598 of the VP1 capsid polypeptide of SEQ ID NO:7.
63. The capsid polypeptide of any one of embodiments 54 to 62, which comprises a valine at a position corresponding to 1601 of the VP1 capsid polypeptide of SEQ ID NO:7.
64. The capsid polypeptide of embodiment 1 or embodiment 2, which comprises a histidine at a position corresponding to R550 of the VP1 capsid polypeptide of SEQ ID NO:7, an alanine at a position corresponding to S576 of the VP1 capsid polypeptide of SEQ ID NO:7, a threonine at a position corresponding to Q579 of SEQ ID NO:7, a lysine at a position corresponding to H584 of the VP1 capsid polypeptide of SEQ ID NO:7, a tyrosine at a position corresponding to Q588 of the VP1 capsid polypeptide of SEQ ID NO:7, an alanine at a position corresponding to W595 of the VP1 capsid polypeptide of SEQ ID NO:7, a leucine at a position corresponding to V596 of the VP1 capsid polypeptide of SEQ ID NO:7, a serine at a position corresponding to N598 of the VP1 capsid polypeptide of SEQ ID NO:7, and a valine at a position corresponding to 1601 of the VP1 capsid polypeptide of SEQ ID NO:7.
65. The capsid polypeptide of embodiment 1 or embodiment 2, which comprises a histidine at a position corresponding to R550 of the VP1 capsid polypeptide of SEQ ID NO:7, an asparagine at a position corresponding to D551 of the VP1 capsid polypeptide of SEQ ID NO:7, an alanine at a position corresponding to S576 of the VP1 capsid polypeptide of SEQ ID NO:7, a threonine at a position corresponding to Q579 of SEQ ID NO:7, a lysine at a position corresponding to H584 of the VP1 capsid polypeptide of SEQ ID NO:7, a tyrosine at a position corresponding to Q588 of the VP1 capsid polypeptide of SEQ ID NO:7, an alanine at a position corresponding to W595 of the VP1 capsid polypeptide of SEQ ID NO:7, a leucine at a position corresponding to V596 of the VP1 capsid polypeptide of SEQ ID NO:7, a serine at a position corresponding to N598 of the VP1 capsid polypeptide of SEQ ID NO:7, and a valine at a position corresponding to 1601 of the VP1 capsid polypeptide of SEQ ID NO:7.
66. The capsid polypeptide of embodiment 1 or embodiment 2, which comprises a histidine at a position corresponding to R550 of the VP1 capsid polypeptide of SEQ ID NO:7, a threonine at a position corresponding to Q579 of the VP1 capsid polypeptide of SEQ ID NO:7, a lysine at a position corresponding to H584 of the VP1 capsid polypeptide of SEQ ID NO:7, an asparagine at a position corresponding to S586 of the VP1 capsid polypeptide of SEQ ID NO:7, a histidine at a position corresponding to A587 of the VP1 capsid polypeptide of SEQ ID NO:7, a threonine at a position corresponding to Q588 of the VP1 capsid polypeptide of SEQ ID NO:7, a glutamine at a position corresponding to A589 of the VP1 capsid polypeptide of SEQ ID NO:7, an alanine at a position corresponding to W595 of the VP1 capsid polypeptide of SEQ ID NO:7, a leucine at a position corresponding to V596 of the VP1 capsid polypeptide of SEQ ID NO:7, a serine at a position corresponding to N598 of the VP1 capsid polypeptide of SEQ ID NO:7, and a valine at a position corresponding to 1601 of the VP1 capsid polypeptide of SEQ ID NO:7.
67. The capsid polypeptide of embodiment 1 or embodiment 2, which comprises an isoleucine at a position corresponding to Q579 of the VP1 capsid polypeptide of SEQ ID NO:7, a serine at a position corresponding to A587 of the VP1 capsid polypeptide of SEQ ID NO:7, an asparagine at a position corresponding to Q588 of the VP1 capsid polypeptide of SEQ ID NO:7, a threonine at a position corresponding to A589 of the VP1 capsid polypeptide of SEQ ID NO:7, an asparagine at a position corresponding to Q592 of the VP1 capsid polypeptide of SEQ ID NO:7, a valine at a position corresponding to T593 of the VP1 capsid polypeptide of SEQ ID NO:7, an alanine at a position corresponding to W595 of the VP1 capsid polypeptide of SEQ ID NO:7, a leucine at a position corresponding to V596 of the VP1 capsid polypeptide of SEQ ID NO:7, a serine at a position corresponding to N598 of the VP1 capsid polypeptide of SEQ ID NO:7, and a valine at a position corresponding to 1601 of the VP1 capsid polypeptide of SEQ ID NO:7
68. A capsid polypeptide comprising the peptide AYGTVATNKQSAY (SEQ ID NO:200) or a peptide whose amino acid sequence has one to five amino acid substitutions as compared to the amino acid sequence of SEQ ID NO:200, provided that the amino acids at positions 1, 4, 9, and 13 of the peptide are alanine, threonine, lysine, and tyrosine, respectively.
69. The capsid polypeptide of embodiment 68, wherein the capsid polypeptide comprises the peptide at a position corresponding to amino acids 576-588 of the VP1 capsid polypeptide of SEQ ID NO:7.
70. A capsid polypeptide comprising the peptide HDNVDADKVMITNEEEIKTTNPVATEAYGTVATNKQSAYAQAQTGALQSQGV (SEQ ID NO:201) or a peptide whose amino acid sequence has one to five amino acid substitutions as compared to the amino acid sequence of SEQ ID NO:201, provided that the amino acids at positions 1, 27, 30, 35, 39, 46, 47, 49, and 52 of the peptide are histidine, alanine, threonine, lysine, tyrosine, alanine, leucine, serine, and valine, respectively.
71. The capsid polypeptide of embodiment 70, wherein the capsid polypeptide comprises the peptide at a position corresponding to amino acids 550-601 of the VP1 capsid polypeptide of SEQ ID NO:7.
72. A capsid polypeptide comprising the peptide HNNVDADKVMITNEEEIKTTNPVATEAYGTVATNKQSAYAQAQTGALQSQGV (SEQ ID NO:202) or a peptide whose amino acid sequence has one to five amino acid substitutions as compared to the amino acid sequence of SEQ ID NO:202, provided that the amino acids at positions 1, 2, 27, 30, 35, 39, 46, 47, 49, and 52 of the peptide are histidine, asparagine, alanine, threonine, lysine, tyrosine, alanine, leucine, serine, and valine, respectively.
73. The capsid polypeptide of embodiment 72, wherein the capsid polypeptide comprises the peptide at a position corresponding to amino acids 550-601 of the VP1 capsid polypeptide of SEQ ID NO:7.
74. A capsid polypeptide comprising the peptide TVATNKQNHT (SEQ ID NO:203) or a peptide whose amino acid sequence has one to five amino acid substitutions as compared to the amino acid sequence of SEQ ID NO:203, provided that the amino acids at positions 1, 6, 8, 9, and 10 of the peptide are threonine, lysine, asparagine, histidine, and threonine, respectively.
75. The capsid polypeptide of embodiment 74, wherein the capsid polypeptide comprises the peptide at a position corresponding to amino acids 579-588 of the VP1 capsid polypeptide of SEQ ID NO:7
76. A capsid polypeptide comprising the peptide HDNVDADKVMITNEEEIKTTNPVATESYGTVATNKQNHTQQAQTGALQSQGV (SEQ ID NO:204) or a peptide whose amino acid sequence has one to five amino acid substitutions as compared to the amino acid sequence of SEQ ID NO:204, provided that the amino acids at positions 1, 30, 35, 37, 38, 39, 40, 46, 47, 49, and 52 of the peptide are histidine, threonine, lysine, asparagine, histidine, threonine, glutamine, alanine, leucine, serine, and valine, respectively.
77. The capsid polypeptide of embodiment 76, wherein the capsid polypeptide comprises the peptide at a position corresponding to amino acids 550-601 of the VP1 capsid polypeptide of SEQ ID NO:7.
78. A capsid polypeptide comprising the peptide IVATNHQSSN (SEQ ID NO:205) or a peptide whose amino acid sequence has one to five amino acid substitutions as compared to the amino acid sequence of SEQ ID NO:205, provided that the amino acids at positions 1, 9, and 10 of the peptide are isoleucine, serine, and asparagine, respectively.
79. The capsid polypeptide of embodiment 78, wherein the capsid polypeptide comprises the peptide at a position corresponding to amino acids 579-588 of the VP1 capsid polypeptide of SEQ ID NO:7
80. A capsid polypeptide comprising the peptide IVATNHQSSNTQANVGALQSQGV (SEQ ID NO:206) or a peptide whose amino acid sequence has one to five amino acid substitutions as compared to the amino acid sequence of SEQ ID NO:206, provided that the amino acids at positions 1, 9, 10, 11, 14, 15, 17, 18, 20, and 23 of the peptide are isoleucine, serine, asparagine, threonine, asparagine, valine, alanine, leucine, serine, and valine, respectively.
81. The capsid polypeptide of embodiment 80, wherein the capsid polypeptide comprises the peptide at a position corresponding to amino acids 579-601 of the VP1 capsid polypeptide of SEQ ID NO:7.
82. The capsid polypeptide of any one of embodiments 68 to 81, wherein at least a portion of the peptide is in a surface loop of the capsid polypeptide.
83. The capsid polypeptide of embodiment 82, wherein the surface loop comprises a VR-VIII variable region.
84. The capsid polypeptide of any one of embodiments 1 to 83, which comprises an amino acid sequence having at least 70% sequence identity ((a) calculated taking targeting peptide insertions into account or (b) calculated without taking targeting peptide insertions into account) to a VP1 capsid polypeptide of SEQ ID NO:1 or the VP2 or VP3 portion thereof.
85. The capsid polypeptide of any one of embodiments 1 to 83, which comprises an amino acid sequence having at least 75% sequence identity ((a) calculated taking targeting peptide insertions into account or (b) calculated without taking targeting peptide insertions into account) to a VP1 capsid polypeptide of SEQ ID NO:1 or the VP2 or VP3 portion thereof.
86. The capsid polypeptide of any one of embodiments 1 to 83, which comprises an amino acid sequence having at least 80% sequence identity ((a) calculated taking targeting peptide insertions into account or (b) calculated without taking targeting peptide insertions into account) to a VP1 capsid polypeptide of SEQ ID NO:1 or the VP2 or VP3 portion thereof.
87. The capsid polypeptide of any one of embodiments 1 to 83, which comprises an amino acid sequence having at least 85% sequence identity ((a) calculated taking targeting peptide insertions into account or (b) calculated without taking targeting peptide insertions into account) to a VP1 capsid polypeptide of SEQ ID NO:1 or the VP2 or VP3 portion thereof.
88. The capsid polypeptide of any one of embodiments 1 to 83, which comprises an amino acid sequence having at least 90% sequence identity ((a) calculated taking targeting peptide insertions into account or (b) calculated without taking targeting peptide insertions into account) to a VP1 capsid polypeptide of SEQ ID NO:1 or the VP2 or VP3 portion thereof.
89. The capsid polypeptide of any one of embodiments 1 to 83, which comprises an amino acid sequence having at least 91% sequence identity ((a) calculated taking targeting peptide insertions into account or (b) calculated without taking targeting peptide insertions into account) to a VP1 capsid polypeptide of SEQ ID NO:1 or the VP2 or VP3 portion thereof.
90. The capsid polypeptide of any one of embodiments 1 to 83, which comprises an amino acid sequence having at least 92% sequence identity ((a) calculated taking targeting peptide insertions into account or (b) calculated without taking targeting peptide insertions into account) to a VP1 capsid polypeptide of SEQ ID NO:1 or the VP2 or VP3 portion thereof.
91. The capsid polypeptide of any one of embodiments 1 to 83, which comprises an amino acid sequence having at least 93% sequence identity ((a) calculated taking targeting peptide insertions into account or (b) calculated without taking targeting peptide insertions into account) to a VP1 capsid polypeptide of SEQ ID NO:1 or the VP2 or VP3 portion thereof.
92. The capsid polypeptide of any one of embodiments 1 to 83, which comprises an amino acid sequence having at least 94% sequence identity ((a) calculated taking targeting peptide insertions into account or (b) calculated without taking targeting peptide insertions into account) to a VP1 capsid polypeptide of SEQ ID NO:1 or the VP2 or VP3 portion thereof.
93. The capsid polypeptide of any one of embodiments 1 to 83, which comprises an amino acid sequence having at least 95% sequence identity ((a) calculated taking targeting peptide insertions into account or (b) calculated without taking targeting peptide insertions into account) to a VP1 capsid polypeptide of SEQ ID NO:1 or the VP2 or VP3 portion thereof.
94. The capsid polypeptide of any one of embodiments 1 to 83, which comprises an amino acid sequence having at least 96% sequence identity ((a) calculated taking targeting peptide insertions into account or (b) calculated without taking targeting peptide insertions into account) to a VP1 capsid polypeptide of SEQ ID NO:1 or the VP2 or VP3 portion thereof.
95. The capsid polypeptide of any one of embodiments 1 to 83, which comprises an amino acid sequence having at least 97% sequence identity ((a) calculated taking targeting peptide insertions into account or (b) calculated without taking targeting peptide insertions into account) to a VP1 capsid polypeptide of SEQ ID NO:1 or the VP2 or VP3 portion thereof.
96. The capsid polypeptide of any one of embodiments 1 to 83, which comprises an amino acid sequence having at least 98% sequence identity ((a) calculated taking targeting peptide insertions into account or (b) calculated without taking targeting peptide insertions into account) to a VP1 capsid polypeptide of SEQ ID NO:1 or the VP2 or VP3 portion thereof.
97. The capsid polypeptide of any one of embodiments 1 to 83, which comprises an amino acid sequence having at least 99% sequence identity ((a) calculated taking targeting peptide insertions into account or (b) calculated without taking targeting peptide insertions into account) to a VP1 capsid polypeptide of SEQ ID NO:1 or the VP2 or VP3 portion thereof.
98. The capsid polypeptide of any one of embodiments 1 to 83, which comprises an amino acid sequence having at least 70% sequence identity ((a) calculated taking targeting peptide insertions into account or (b) calculated without taking targeting peptide insertions into account) to a VP1 capsid polypeptide of SEQ ID NO:3 or the VP2 or VP3 portion thereof.
99. The capsid polypeptide of any one of embodiments 1 to 83, which comprises an amino acid sequence having at least 75% sequence identity ((a) calculated taking targeting peptide insertions into account or (b) calculated without taking targeting peptide insertions into account) to a VP1 capsid polypeptide of SEQ ID NO:3 or the VP2 or VP3 portion thereof.
100. The capsid polypeptide of any one of embodiments 1 to 83, which comprises an amino acid sequence having at least 80% sequence identity ((a) calculated taking targeting peptide insertions into account or (b) calculated without taking targeting peptide insertions into account) to a VP1 capsid polypeptide of SEQ ID NO:3 or the VP2 or VP3 portion thereof.
101. The capsid polypeptide of any one of embodiments 1 to 83, which comprises an amino acid sequence having at least 85% sequence identity ((a) calculated taking targeting peptide insertions into account or (b) calculated without taking targeting peptide insertions into account) to a VP1 capsid polypeptide of SEQ ID NO:3 or the VP2 or VP3 portion thereof.
102. The capsid polypeptide of any one of embodiments 1 to 83, which comprises an amino acid sequence having at least 90% sequence identity ((a) calculated taking targeting peptide insertions into account or (b) calculated without taking targeting peptide insertions into account) to a VP1 capsid polypeptide of SEQ ID NO:3 or the VP2 or VP3 portion thereof.
103. The capsid polypeptide of any one of embodiments 1 to 83, which comprises an amino acid sequence having at least 91% sequence identity ((a) calculated taking targeting peptide insertions into account or (b) calculated without taking targeting peptide insertions into account) to a VP1 capsid polypeptide of SEQ ID NO:3 or the VP2 or VP3 portion thereof.
104. The capsid polypeptide of any one of embodiments 1 to 83, which comprises an amino acid sequence having at least 92% sequence identity ((a) calculated taking targeting peptide insertions into account or (b) calculated without taking targeting peptide insertions into account) to a VP1 capsid polypeptide of SEQ ID NO:3 or the VP2 or VP3 portion thereof.
105. The capsid polypeptide of any one of embodiments 1 to 83, which comprises an amino acid sequence having at least 93% sequence identity ((a) calculated taking targeting peptide insertions into account or (b) calculated without taking targeting peptide insertions into account) to a VP1 capsid polypeptide of SEQ ID NO:3 or the VP2 or VP3 portion thereof.
106. The capsid polypeptide of any one of embodiments 1 to 83, which comprises an amino acid sequence having at least 94% sequence identity ((a) calculated taking targeting peptide insertions into account or (b) calculated without taking targeting peptide insertions into account) to a VP1 capsid polypeptide of SEQ ID NO:3 or the VP2 or VP3 portion thereof.
107. The capsid polypeptide of any one of embodiments 1 to 83, which comprises an amino acid sequence having at least 95% sequence identity ((a) calculated taking targeting peptide insertions into account or (b) calculated without taking targeting peptide insertions into account) to a VP1 capsid polypeptide of SEQ ID NO:3 or the VP2 or VP3 portion thereof.
108. The capsid polypeptide of any one of embodiments 1 to 83, which comprises an amino acid sequence having at least 96% sequence identity ((a) calculated taking targeting peptide insertions into account or (b) calculated without taking targeting peptide insertions into account) to a VP1 capsid polypeptide of SEQ ID NO:3 or the VP2 or VP3 portion thereof.
109. The capsid polypeptide of any one of embodiments 1 to 83, which comprises an amino acid sequence having at least 97% sequence identity ((a) calculated taking targeting peptide insertions into account or (b) calculated without taking targeting peptide insertions into account) to a VP1 capsid polypeptide of SEQ ID NO:3 or the VP2 or VP3 portion thereof.
110. The capsid polypeptide of any one of embodiments 1 to 83, which comprises an amino acid sequence having at least 98% sequence identity ((a) calculated taking targeting peptide insertions into account or (b) calculated without taking targeting peptide insertions into account) to a VP1 capsid polypeptide of SEQ ID NO:3 or the VP2 or VP3 portion thereof.
111. The capsid polypeptide of any one of embodiments 1 to 83, which comprises an amino acid sequence having at least 99% sequence identity ((a) calculated taking targeting peptide insertions into account or (b) calculated without taking targeting peptide insertions into account) to a VP1 capsid polypeptide of SEQ ID NO:3 or the VP2 or VP3 portion thereof.
112. The capsid polypeptide of any one of embodiments 1 to 83, which comprises an amino acid sequence having at least 70% sequence identity ((a) calculated taking targeting peptide insertions into account or (b) calculated without taking targeting peptide insertions into account) to a VP1 capsid polypeptide of SEQ ID NO:5 or the VP2 or VP3 portion thereof.
113. The capsid polypeptide of any one of embodiments 1 to 83, which comprises an amino acid sequence having at least 75% sequence identity ((a) calculated taking targeting peptide insertions into account or (b) calculated without taking targeting peptide insertions into account) to a VP1 capsid polypeptide of SEQ ID NO:5 or the VP2 or VP3 portion thereof.
114. The capsid polypeptide of any one of embodiments 1 to 83, which comprises an amino acid sequence having at least 80% sequence identity ((a) calculated taking targeting peptide insertions into account or (b) calculated without taking targeting peptide insertions into account) to a VP1 capsid polypeptide of SEQ ID NO:5 or the VP2 or VP3 portion thereof.
115. The capsid polypeptide of any one of embodiments 1 to 83, which comprises an amino acid sequence having at least 85% sequence identity ((a) calculated taking targeting peptide insertions into account or (b) calculated without taking targeting peptide insertions into account) to a VP1 capsid polypeptide of SEQ ID NO:5 or the VP2 or VP3 portion thereof.
116. The capsid polypeptide of any one of embodiments 1 to 83, which comprises an amino acid sequence having at least 90% sequence identity ((a) calculated taking targeting peptide insertions into account or (b) calculated without taking targeting peptide insertions into account) to a VP1 capsid polypeptide of SEQ ID NO:5 or the VP2 or VP3 portion thereof.
117. The capsid polypeptide of any one of embodiments 1 to 83, which comprises an amino acid sequence having at least 91% sequence identity ((a) calculated taking targeting peptide insertions into account or (b) calculated without taking targeting peptide insertions into account) to a VP1 capsid polypeptide of SEQ ID NO:5 or the VP2 or VP3 portion thereof.
118. The capsid polypeptide of any one of embodiments 1 to 83, which comprises an amino acid sequence having at least 92% sequence identity ((a) calculated taking targeting peptide insertions into account or (b) calculated without taking targeting peptide insertions into account) to a VP1 capsid polypeptide of SEQ ID NO:5 or the VP2 or VP3 portion thereof.
119. The capsid polypeptide of any one of embodiments 1 to 83, which comprises an amino acid sequence having at least 93% sequence identity ((a) calculated taking targeting peptide insertions into account or (b) calculated without taking targeting peptide insertions into account) to a VP1 capsid polypeptide of SEQ ID NO:5 or the VP2 or VP3 portion thereof.
120. The capsid polypeptide of any one of embodiments 1 to 83, which comprises an amino acid sequence having at least 94% sequence identity ((a) calculated taking targeting peptide insertions into account or (b) calculated without taking targeting peptide insertions into account) to a VP1 capsid polypeptide of SEQ ID NO:5 or the VP2 or VP3 portion thereof.
121. The capsid polypeptide of any one of embodiments 1 to 83, which comprises an amino acid sequence having at least 95% sequence identity ((a) calculated taking targeting peptide insertions into account or (b) calculated without taking targeting peptide insertions into account) to a VP1 capsid polypeptide of SEQ ID NO:5 or the VP2 or VP3 portion thereof.
122. The capsid polypeptide of any one of embodiments 1 to 83, which comprises an amino acid sequence having at least 96% sequence identity ((a) calculated taking targeting peptide insertions into account or (b) calculated without taking targeting peptide insertions into account) to a VP1 capsid polypeptide of SEQ ID NO:5 or the VP2 or VP3 portion thereof.
123. The capsid polypeptide of any one of embodiments 1 to 83, which comprises an amino acid sequence having at least 97% sequence identity ((a) calculated taking targeting peptide insertions into account or (b) calculated without taking targeting peptide insertions into account) to a VP1 capsid polypeptide of SEQ ID NO:5 or the VP2 or VP3 portion thereof.
124. The capsid polypeptide of any one of embodiments 1 to 83, which comprises an amino acid sequence having at least 98% sequence identity ((a) calculated taking targeting peptide insertions into account or (b) calculated without taking targeting peptide insertions into account) to a VP1 capsid polypeptide of SEQ ID NO:5 or the VP2 or VP3 portion thereof.
125. The capsid polypeptide of any one of embodiments 1 to 83, which comprises an amino acid sequence having at least 99% sequence identity ((a) calculated taking targeting peptide insertions into account or (b) calculated without taking targeting peptide insertions into account) to a VP1 capsid polypeptide of SEQ ID NO:5 or the VP2 or VP3 portion thereof.
126. The capsid polypeptide of any one of embodiments 1 to 83, which comprises an amino acid sequence having at least 70% sequence identity ((a) calculated taking targeting peptide insertions into account or (b) calculated without taking targeting peptide insertions into account) to a VP1 capsid polypeptide of SEQ ID NO:7 or the VP2 or VP3 portion thereof.
127. The capsid polypeptide of any one of embodiments 1 to 83, which comprises an amino acid sequence having at least 75% sequence identity ((a) calculated taking targeting peptide insertions into account or (b) calculated without taking targeting peptide insertions into account) to a VP1 capsid polypeptide of SEQ ID NO:7 or the VP2 or VP3 portion thereof.
128. The capsid polypeptide of any one of embodiments 1 to 83, which comprises an amino acid sequence having at least 80% sequence identity ((a) calculated taking targeting peptide insertions into account or (b) calculated without taking targeting peptide insertions into account) to a VP1 capsid polypeptide of SEQ ID NO:7 or the VP2 or VP3 portion thereof.
129. The capsid polypeptide of any one of embodiments 1 to 83, which comprises an amino acid sequence having at least 85% sequence identity ((a) calculated taking targeting peptide insertions into account or (b) calculated without taking targeting peptide insertions into account) to a VP1 capsid polypeptide of SEQ ID NO:7 or the VP2 or VP3 portion thereof.
130. The capsid polypeptide of any one of embodiments 1 to 83, which comprises an amino acid sequence having at least 90% sequence identity ((a) calculated taking targeting peptide insertions into account or (b) calculated without taking targeting peptide insertions into account) to a VP1 capsid polypeptide of SEQ ID NO:7 or the VP2 or VP3 portion thereof.
131. The capsid polypeptide of any one of embodiments 1 to 83, which comprises an amino acid sequence having at least 91% sequence identity ((a) calculated taking targeting peptide insertions into account or (b) calculated without taking targeting peptide insertions into account) to a VP1 capsid polypeptide of SEQ ID NO:7 or the VP2 or VP3 portion thereof.
132. The capsid polypeptide of any one of embodiments 1 to 83, which comprises an amino acid sequence having at least 92% sequence identity ((a) calculated taking targeting peptide insertions into account or (b) calculated without taking targeting peptide insertions into account) to a VP1 capsid polypeptide of SEQ ID NO:7 or the VP2 or VP3 portion thereof.
133. The capsid polypeptide of any one of embodiments 1 to 83, which comprises an amino acid sequence having at least 93% sequence identity ((a) calculated taking targeting peptide insertions into account or (b) calculated without taking targeting peptide insertions into account) to a VP1 capsid polypeptide of SEQ ID NO:7 or the VP2 or VP3 portion thereof.
134. The capsid polypeptide of any one of embodiments 1 to 83, which comprises an amino acid sequence having at least 94% sequence identity ((a) calculated taking targeting peptide insertions into account or (b) calculated without taking targeting peptide insertions into account) to a VP1 capsid polypeptide of SEQ ID NO:7 or the VP2 or VP3 portion thereof.
135. The capsid polypeptide of any one of embodiments 1 to 83, which comprises an amino acid sequence having at least 95% sequence identity ((a) calculated taking targeting peptide insertions into account or (b) calculated without taking targeting peptide insertions into account) to a VP1 capsid polypeptide of SEQ ID NO:7 or the VP2 or VP3 portion thereof.
136. The capsid polypeptide of any one of embodiments 1 to 83, which comprises an amino acid sequence having at least 96% sequence identity ((a) calculated taking targeting peptide insertions into account or (b) calculated without taking targeting peptide insertions into account) to a VP1 capsid polypeptide of SEQ ID NO:7 or the VP2 or VP3 portion thereof.
137. The capsid polypeptide of any one of embodiments 1 to 83, which comprises an amino acid sequence having at least 97% sequence identity ((a) calculated taking targeting peptide insertions into account or (b) calculated without taking targeting peptide insertions into account) to a VP1 capsid polypeptide of SEQ ID NO:7 or the VP2 or VP3 portion thereof.
138. The capsid polypeptide of any one of embodiments 1 to 83, which comprises an amino acid sequence having at least 98% sequence identity ((a) calculated taking targeting peptide insertions into account or (b) calculated without taking targeting peptide insertions into account) to a VP1 capsid polypeptide of SEQ ID NO:7 or the VP2 or VP3 portion thereof.
139. The capsid polypeptide of any one of embodiments 1 to 83, which comprises an amino acid sequence having at least 99% sequence identity ((a) calculated taking targeting peptide insertions into account or (b) calculated without taking targeting peptide insertions into account) to a VP1 capsid polypeptide of SEQ ID NO:7 or the VP2 or VP3 portion thereof.
140. The capsid polypeptide of any one of embodiments 1 to 83, which comprises an amino acid sequence having at least 70% sequence identity ((a) calculated taking targeting peptide insertions into account or (b) calculated without taking targeting peptide insertions into account) to a VP1 capsid polypeptide of SEQ ID NO:9 or the VP2 or VP3 portion thereof.
141. The capsid polypeptide of any one of embodiments 1 to 83, which comprises an amino acid sequence having at least 75% sequence identity ((a) calculated taking targeting peptide insertions into account or (b) calculated without taking targeting peptide insertions into account) to a VP1 capsid polypeptide of SEQ ID NO:9 or the VP2 or VP3 portion thereof.
142. The capsid polypeptide of any one of embodiments 1 to 83, which comprises an amino acid sequence having at least 80% sequence identity ((a) calculated taking targeting peptide insertions into account or (b) calculated without taking targeting peptide insertions into account) to a VP1 capsid polypeptide of SEQ ID NO:9 or the VP2 or VP3 portion thereof.
143. The capsid polypeptide of any one of embodiments 1 to 83, which comprises an amino acid sequence having at least 85% sequence identity ((a) calculated taking targeting peptide insertions into account or (b) calculated without taking targeting peptide insertions into account) to a VP1 capsid polypeptide of SEQ ID NO:9 or the VP2 or VP3 portion thereof.
144. The capsid polypeptide of any one of embodiments 1 to 83, which comprises an amino acid sequence having at least 90% sequence identity ((a) calculated taking targeting peptide insertions into account or (b) calculated without taking targeting peptide insertions into account) to a VP1 capsid polypeptide of SEQ ID NO:9 or the VP2 or VP3 portion thereof.
145. The capsid polypeptide of any one of embodiments 1 to 83, which comprises an amino acid sequence having at least 91% sequence identity ((a) calculated taking targeting peptide insertions into account or (b) calculated without taking targeting peptide insertions into account) to a VP1 capsid polypeptide of SEQ ID NO:9 or the VP2 or VP3 portion thereof.
146. The capsid polypeptide of any one of embodiments 1 to 83, which comprises an amino acid sequence having at least 92% sequence identity ((a) calculated taking targeting peptide insertions into account or (b) calculated without taking targeting peptide insertions into account) to a VP1 capsid polypeptide of SEQ ID NO:9 or the VP2 or VP3 portion thereof.
147. The capsid polypeptide of any one of embodiments 1 to 83, which comprises an amino acid sequence having at least 93% sequence identity ((a) calculated taking targeting peptide insertions into account or (b) calculated without taking targeting peptide insertions into account) to a VP1 capsid polypeptide of SEQ ID NO:9 or the VP2 or VP3 portion thereof.
148. The capsid polypeptide of any one of embodiments 1 to 83, which comprises an amino acid sequence having at least 94% sequence identity ((a) calculated taking targeting peptide insertions into account or (b) calculated without taking targeting peptide insertions into account) to a VP1 capsid polypeptide of SEQ ID NO:9 or the VP2 or VP3 portion thereof.
149. The capsid polypeptide of any one of embodiments 1 to 83, which comprises an amino acid sequence having at least 95% sequence identity ((a) calculated taking targeting peptide insertions into account or (b) calculated without taking targeting peptide insertions into account) to a VP1 capsid polypeptide of SEQ ID NO:9 or the VP2 or VP3 portion thereof.
150. The capsid polypeptide of any one of embodiments 1 to 83, which comprises an amino acid sequence having at least 96% sequence identity ((a) calculated taking targeting peptide insertions into account or (b) calculated without taking targeting peptide insertions into account) to a VP1 capsid polypeptide of SEQ ID NO:9 or the VP2 or VP3 portion thereof.
151. The capsid polypeptide of any one of embodiments 1 to 83, which comprises an amino acid sequence having at least 97% sequence identity ((a) calculated taking targeting peptide insertions into account or (b) calculated without taking targeting peptide insertions into account) to a VP1 capsid polypeptide of SEQ ID NO:9 or the VP2 or VP3 portion thereof.
152. The capsid polypeptide of any one of embodiments 1 to 83, which comprises an amino acid sequence having at least 98% sequence identity ((a) calculated taking targeting peptide insertions into account or (b) calculated without taking targeting peptide insertions into account) to a VP1 capsid polypeptide of SEQ ID NO:9 or the VP2 or VP3 portion thereof.
153. The capsid polypeptide of any one of embodiments 1 to 83, which comprises an amino acid sequence having at least 99% sequence identity ((a) calculated taking targeting peptide insertions into account or (b) calculated without taking targeting peptide insertions into account) to a VP1 capsid polypeptide of SEQ ID NO:9 or the VP2 or VP3 portion thereof.
154. The capsid polypeptide of any one of embodiments 1 to 83, which comprises an amino acid sequence having at least 70% sequence identity ((a) calculated taking targeting peptide insertions into account or (b) calculated without taking targeting peptide insertions into account) to a VP1 capsid polypeptide of SEQ ID NO:11 or the VP2 or VP3 portion thereof.
155. The capsid polypeptide of any one of embodiments 1 to 83, which comprises an amino acid sequence having at least 75% sequence identity ((a) calculated taking targeting peptide insertions into account or (b) calculated without taking targeting peptide insertions into account) to a VP1 capsid polypeptide of SEQ ID NO:11 or the VP2 or VP3 portion thereof.
156. The capsid polypeptide of any one of embodiments 1 to 83, which comprises an amino acid sequence having at least 80% sequence identity ((a) calculated taking targeting peptide insertions into account or (b) calculated without taking targeting peptide insertions into account) to a VP1 capsid polypeptide of SEQ ID NO:11 or the VP2 or VP3 portion thereof.
157. The capsid polypeptide of any one of embodiments 1 to 83, which comprises an amino acid sequence having at least 85% sequence identity ((a) calculated taking targeting peptide insertions into account or (b) calculated without taking targeting peptide insertions into account) to a VP1 capsid polypeptide of SEQ ID NO:11 or the VP2 or VP3 portion thereof.
158. The capsid polypeptide of any one of embodiments 1 to 83, which comprises an amino acid sequence having at least 90% sequence identity ((a) calculated taking targeting peptide insertions into account or (b) calculated without taking targeting peptide insertions into account) to a VP1 capsid polypeptide of SEQ ID NO:11 or the VP2 or VP3 portion thereof.
159. The capsid polypeptide of any one of embodiments 1 to 83, which comprises an amino acid sequence having at least 91% sequence identity ((a) calculated taking targeting peptide insertions into account or (b) calculated without taking targeting peptide insertions into account) to a VP1 capsid polypeptide of SEQ ID NO:11 or the VP2 or VP3 portion thereof.
160. The capsid polypeptide of any one of embodiments 1 to 83, which comprises an amino acid sequence having at least 92% sequence identity ((a) calculated taking targeting peptide insertions into account or (b) calculated without taking targeting peptide insertions into account) to a VP1 capsid polypeptide of SEQ ID NO:11 or the VP2 or VP3 portion thereof.
161. The capsid polypeptide of any one of embodiments 1 to 83, which comprises an amino acid sequence having at least 93% sequence identity ((a) calculated taking targeting peptide insertions into account or (b) calculated without taking targeting peptide insertions into account) to a VP1 capsid polypeptide of SEQ ID NO:11 or the VP2 or VP3 portion thereof.
162. The capsid polypeptide of any one of embodiments 1 to 83, which comprises an amino acid sequence having at least 94% sequence identity ((a) calculated taking targeting peptide insertions into account or (b) calculated without taking targeting peptide insertions into account) to a VP1 capsid polypeptide of SEQ ID NO:11 or the VP2 or VP3 portion thereof.
163. The capsid polypeptide of any one of embodiments 1 to 83, which comprises an amino acid sequence having at least 95% sequence identity ((a) calculated taking targeting peptide insertions into account or (b) calculated without taking targeting peptide insertions into account) to a VP1 capsid polypeptide of SEQ ID NO:11 or the VP2 or VP3 portion thereof.
164. The capsid polypeptide of any one of embodiments 1 to 83, which comprises an amino acid sequence having at least 96% sequence identity ((a) calculated taking targeting peptide insertions into account or (b) calculated without taking targeting peptide insertions into account) to a VP1 capsid polypeptide of SEQ ID NO:11 or the VP2 or VP3 portion thereof.
165. The capsid polypeptide of any one of embodiments 1 to 83, which comprises an amino acid sequence having at least 97% sequence identity ((a) calculated taking targeting peptide insertions into account or (b) calculated without taking targeting peptide insertions into account) to a VP1 capsid polypeptide of SEQ ID NO: 11 or the VP2 or VP3 portion thereof.
166. The capsid polypeptide of any one of embodiments 1 to 83, which comprises an amino acid sequence having at least 98% sequence identity ((a) calculated taking targeting peptide insertions into account or (b) calculated without taking targeting peptide insertions into account) to a VP1 capsid polypeptide of SEQ ID NO:11 or the VP2 or VP3 portion thereof.
167. The capsid polypeptide of any one of embodiments 1 to 83, which comprises an amino acid sequence having at least 99% sequence identity ((a) calculated taking targeting peptide insertions into account or (b) calculated without taking targeting peptide insertions into account) to a VP1 capsid polypeptide of SEQ ID NO:11 or the VP2 or VP3 portion thereof.
168. The capsid polypeptide of any one of embodiments 1 to 83, which comprises an amino acid sequence having at least 70% sequence identity ((a) calculated taking targeting peptide insertions into account or (b) calculated without taking targeting peptide insertions into account) to a VP1 capsid polypeptide of SEQ ID NO:12 or the VP2 or VP3 portion thereof.
169. The capsid polypeptide of any one of embodiments 1 to 83, which comprises an amino acid sequence having at least 75% sequence identity ((a) calculated taking targeting peptide insertions into account or (b) calculated without taking targeting peptide insertions into account) to a VP1 capsid polypeptide of SEQ ID NO:12 or the VP2 or VP3 portion thereof.
170. The capsid polypeptide of any one of embodiments 1 to 83, which comprises an amino acid sequence having at least 80% sequence identity ((a) calculated taking targeting peptide insertions into account or (b) calculated without taking targeting peptide insertions into account) to a VP1 capsid polypeptide of SEQ ID NO:12 or the VP2 or VP3 portion thereof.
171. The capsid polypeptide of any one of embodiments 1 to 83, which comprises an amino acid sequence having at least 85% sequence identity ((a) calculated taking targeting peptide insertions into account or (b) calculated without taking targeting peptide insertions into account) to a VP1 capsid polypeptide of SEQ ID NO:12 or the VP2 or VP3 portion thereof.
172. The capsid polypeptide of any one of embodiments 1 to 83, which comprises an amino acid sequence having at least 90% sequence identity ((a) calculated taking targeting peptide insertions into account or (b) calculated without taking targeting peptide insertions into account) to a VP1 capsid polypeptide of SEQ ID NO:12 or the VP2 or VP3 portion thereof.
173. The capsid polypeptide of any one of embodiments 1 to 83, which comprises an amino acid sequence having at least 91% sequence identity ((a) calculated taking targeting peptide insertions into account or (b) calculated without taking targeting peptide insertions into account) to a VP1 capsid polypeptide of SEQ ID NO:12 or the VP2 or VP3 portion thereof.
174. The capsid polypeptide of any one of embodiments 1 to 83, which comprises an amino acid sequence having at least 92% sequence identity ((a) calculated taking targeting peptide insertions into account or (b) calculated without taking targeting peptide insertions into account) to a VP1 capsid polypeptide of SEQ ID NO:12 or the VP2 or VP3 portion thereof.
175. The capsid polypeptide of any one of embodiments 1 to 83, which comprises an amino acid sequence having at least 93% sequence identity ((a) calculated taking targeting peptide insertions into account or (b) calculated without taking targeting peptide insertions into account) to a VP1 capsid polypeptide of SEQ ID NO:12 or the VP2 or VP3 portion thereof.
176. The capsid polypeptide of any one of embodiments 1 to 83, which comprises an amino acid sequence having at least 94% sequence identity ((a) calculated taking targeting peptide insertions into account or (b) calculated without taking targeting peptide insertions into account) to a VP1 capsid polypeptide of SEQ ID NO:12 or the VP2 or VP3 portion thereof.
177. The capsid polypeptide of any one of embodiments 1 to 83, which comprises an amino acid sequence having at least 95% sequence identity ((a) calculated taking targeting peptide insertions into account or (b) calculated without taking targeting peptide insertions into account) to a VP1 capsid polypeptide of SEQ ID NO:12 or the VP2 or VP3 portion thereof.
178. The capsid polypeptide of any one of embodiments 1 to 83, which comprises an amino acid sequence having at least 96% sequence identity ((a) calculated taking targeting peptide insertions into account or (b) calculated without taking targeting peptide insertions into account) to a VP1 capsid polypeptide of SEQ ID NO:12 or the VP2 or VP3 portion thereof.
179. The capsid polypeptide of any one of embodiments 1 to 83, which comprises an amino acid sequence having at least 97% sequence identity ((a) calculated taking targeting peptide insertions into account or (b) calculated without taking targeting peptide insertions into account) to a VP1 capsid polypeptide of SEQ ID NO:12 or the VP2 or VP3 portion thereof.
180. The capsid polypeptide of any one of embodiments 1 to 83, which comprises an amino acid sequence having at least 98% sequence identity ((a) calculated taking targeting peptide insertions into account or (b) calculated without taking targeting peptide insertions into account) to a VP1 capsid polypeptide of SEQ ID NO:12 or the VP2 or VP3 portion thereof.
181. The capsid polypeptide of any one of embodiments 1 to 83, which comprises an amino acid sequence having at least 99% sequence identity ((a) calculated taking targeting peptide insertions into account or (b) calculated without taking targeting peptide insertions into account) to a VP1 capsid polypeptide of SEQ ID NO:12 or the VP2 or VP3 portion thereof.
182. A capsid polypeptide, which is optionally a capsid polypeptide according to any one of embodiments 1 to 83, which comprises the amino acid sequence of a VP1 capsid polypeptide of SEQ ID NO:12 or the VP2 or VP3 portion thereof.
183. The capsid polypeptide of any one of embodiments 1 to 83, which comprises an amino acid sequence having at least 70% sequence identity ((a) calculated taking targeting peptide insertions into account or (b) calculated without taking targeting peptide insertions into account) to a VP1 capsid polypeptide of SEQ ID NO:14 or the VP2 or VP3 portion thereof.
184. The capsid polypeptide of any one of embodiments 1 to 83, which comprises an amino acid sequence having at least 75% sequence identity ((a) calculated taking targeting peptide insertions into account or (b) calculated without taking targeting peptide insertions into account) to a VP1 capsid polypeptide of SEQ ID NO:14 or the VP2 or VP3 portion thereof.
185. The capsid polypeptide of any one of embodiments 1 to 83, which comprises an amino acid sequence having at least 80% sequence identity ((a) calculated taking targeting peptide insertions into account or (b) calculated without taking targeting peptide insertions into account) to a VP1 capsid polypeptide of SEQ ID NO:14 or the VP2 or VP3 portion thereof.
186. The capsid polypeptide of any one of embodiments 1 to 83, which comprises an amino acid sequence having at least 85% sequence identity ((a) calculated taking targeting peptide insertions into account or (b) calculated without taking targeting peptide insertions into account) to a VP1 capsid polypeptide of SEQ ID NO:14 or the VP2 or VP3 portion thereof.
187. The capsid polypeptide of any one of embodiments 1 to 83, which comprises an amino acid sequence having at least 90% sequence identity ((a) calculated taking targeting peptide insertions into account or (b) calculated without taking targeting peptide insertions into account) to a VP1 capsid polypeptide of SEQ ID NO:14 or the VP2 or VP3 portion thereof.
188. The capsid polypeptide of any one of embodiments 1 to 83, which comprises an amino acid sequence having at least 91% sequence identity ((a) calculated taking targeting peptide insertions into account or (b) calculated without taking targeting peptide insertions into account) to a VP1 capsid polypeptide of SEQ ID NO:14 or the VP2 or VP3 portion thereof.
189. The capsid polypeptide of any one of embodiments 1 to 83, which comprises an amino acid sequence having at least 92% sequence identity ((a) calculated taking targeting peptide insertions into account or (b) calculated without taking targeting peptide insertions into account) to a VP1 capsid polypeptide of SEQ ID NO:14 or the VP2 or VP3 portion thereof.
190. The capsid polypeptide of any one of embodiments 1 to 83, which comprises an amino acid sequence having at least 93% sequence identity ((a) calculated taking targeting peptide insertions into account or (b) calculated without taking targeting peptide insertions into account) to a VP1 capsid polypeptide of SEQ ID NO:14 or the VP2 or VP3 portion thereof.
191. The capsid polypeptide of any one of embodiments 1 to 83, which comprises an amino acid sequence having at least 94% sequence identity ((a) calculated taking targeting peptide insertions into account or (b) calculated without taking targeting peptide insertions into account) to a VP1 capsid polypeptide of SEQ ID NO:14 or the VP2 or VP3 portion thereof.
192. The capsid polypeptide of any one of embodiments 1 to 83, which comprises an amino acid sequence having at least 95% sequence identity ((a) calculated taking targeting peptide insertions into account or (b) calculated without taking targeting peptide insertions into account) to a VP1 capsid polypeptide of SEQ ID NO:14 or the VP2 or VP3 portion thereof.
193. The capsid polypeptide of any one of embodiments 1 to 83, which comprises an amino acid sequence having at least 96% sequence identity ((a) calculated taking targeting peptide insertions into account or (b) calculated without taking targeting peptide insertions into account) to a VP1 capsid polypeptide of SEQ ID NO:14 or the VP2 or VP3 portion thereof.
194. The capsid polypeptide of any one of embodiments 1 to 83, which comprises an amino acid sequence having at least 97% sequence identity ((a) calculated taking targeting peptide insertions into account or (b) calculated without taking targeting peptide insertions into account) to a VP1 capsid polypeptide of SEQ ID NO:14 or the VP2 or VP3 portion thereof.
195. The capsid polypeptide of any one of embodiments 1 to 83, which comprises an amino acid sequence having at least 98% sequence identity ((a) calculated taking targeting peptide insertions into account or (b) calculated without taking targeting peptide insertions into account) to a VP1 capsid polypeptide of SEQ ID NO:14 or the VP2 or VP3 portion thereof.
196. The capsid polypeptide of any one of embodiments 1 to 83, which comprises an amino acid sequence having at least 99% sequence identity ((a) calculated taking targeting peptide insertions into account or (b) calculated without taking targeting peptide insertions into account) to a VP1 capsid polypeptide of SEQ ID NO:14 or the VP2 or VP3 portion thereof.
197. A capsid polypeptide, which is optionally a capsid polypeptide according to any one of embodiments 1 to 83, which comprises the amino acid sequence of a VP1 capsid polypeptide of SEQ ID NO:14 or the VP2 or VP3 portion thereof.
198. The capsid polypeptide of any one of embodiments 1 to 83, which comprises an amino acid sequence having at least 70% sequence identity ((a) calculated taking targeting peptide insertions into account or (b) calculated without taking targeting peptide insertions into account) to a VP1 capsid polypeptide of SEQ ID NO:16 or the VP2 or VP3 portion thereof.
199. The capsid polypeptide of any one of embodiments 1 to 83, which comprises an amino acid sequence having at least 75% sequence identity ((a) calculated taking targeting peptide insertions into account or (b) calculated without taking targeting peptide insertions into account) to a VP1 capsid polypeptide of SEQ ID NO:16 or the VP2 or VP3 portion thereof.
200. The capsid polypeptide of any one of embodiments 1 to 83, which comprises an amino acid sequence having at least 80% sequence identity ((a) calculated taking targeting peptide insertions into account or (b) calculated without taking targeting peptide insertions into account) to a VP1 capsid polypeptide of SEQ ID NO:16 or the VP2 or VP3 portion thereof.
201. The capsid polypeptide of any one of embodiments 1 to 83, which comprises an amino acid sequence having at least 85% sequence identity ((a) calculated taking targeting peptide insertions into account or (b) calculated without taking targeting peptide insertions into account) to a VP1 capsid polypeptide of SEQ ID NO:16 or the VP2 or VP3 portion thereof.
202. The capsid polypeptide of any one of embodiments 1 to 83, which comprises an amino acid sequence having at least 90% sequence identity ((a) calculated taking targeting peptide insertions into account or (b) calculated without taking targeting peptide insertions into account) to a VP1 capsid polypeptide of SEQ ID NO:16 or the VP2 or VP3 portion thereof.
203. The capsid polypeptide of any one of embodiments 1 to 83, which comprises an amino acid sequence having at least 91% sequence identity ((a) calculated taking targeting peptide insertions into account or (b) calculated without taking targeting peptide insertions into account) to a VP1 capsid polypeptide of SEQ ID NO:16 or the VP2 or VP3 portion thereof.
204. The capsid polypeptide of any one of embodiments 1 to 83, which comprises an amino acid sequence having at least 92% sequence identity ((a) calculated taking targeting peptide insertions into account or (b) calculated without taking targeting peptide insertions into account) to a VP1 capsid polypeptide of SEQ ID NO:16 or the VP2 or VP3 portion thereof.
205. The capsid polypeptide of any one of embodiments 1 to 83, which comprises an amino acid sequence having at least 93% sequence identity ((a) calculated taking targeting peptide insertions into account or (b) calculated without taking targeting peptide insertions into account) to a VP1 capsid polypeptide of SEQ ID NO:16 or the VP2 or VP3 portion thereof.
206. The capsid polypeptide of any one of embodiments 1 to 83, which comprises an amino acid sequence having at least 94% sequence identity ((a) calculated taking targeting peptide insertions into account or (b) calculated without taking targeting peptide insertions into account) to a VP1 capsid polypeptide of SEQ ID NO:16 or the VP2 or VP3 portion thereof.
207. The capsid polypeptide of any one of embodiments 1 to 83, which comprises an amino acid sequence having at least 95% sequence identity ((a) calculated taking targeting peptide insertions into account or (b) calculated without taking targeting peptide insertions into account) to a VP1 capsid polypeptide of SEQ ID NO:16 or the VP2 or VP3 portion thereof.
208. The capsid polypeptide of any one of embodiments 1 to 83, which comprises an amino acid sequence having at least 96% sequence identity ((a) calculated taking targeting peptide insertions into account or (b) calculated without taking targeting peptide insertions into account) to a VP1 capsid polypeptide of SEQ ID NO:16 or the VP2 or VP3 portion thereof.
209. The capsid polypeptide of any one of embodiments 1 to 83, which comprises an amino acid sequence having at least 97% sequence identity ((a) calculated taking targeting peptide insertions into account or (b) calculated without taking targeting peptide insertions into account) to a VP1 capsid polypeptide of SEQ ID NO:16 or the VP2 or VP3 portion thereof.
210. The capsid polypeptide of any one of embodiments 1 to 83, which comprises an amino acid sequence having at least 98% sequence identity ((a) calculated taking targeting peptide insertions into account or (b) calculated without taking targeting peptide insertions into account) to a VP1 capsid polypeptide of SEQ ID NO:16 or the VP2 or VP3 portion thereof.
211. The capsid polypeptide of any one of embodiments 1 to 83, which comprises an amino acid sequence having at least 99% sequence identity ((a) calculated taking targeting peptide insertions into account or (b) calculated without taking targeting peptide insertions into account) to a VP1 capsid polypeptide of SEQ ID NO:16 or the VP2 or VP3 portion thereof.
212. A capsid polypeptide, which is optionally a capsid polypeptide according to any one of embodiments 1 to 83, which comprises the amino acid sequence of a VP1 capsid polypeptide of SEQ ID NO:16 or the VP2 or VP3 portion thereof.
213. The capsid polypeptide of any one of embodiments 1 to 83, which comprises an amino acid sequence having at least 70% sequence identity ((a) calculated taking targeting peptide insertions into account or (b) calculated without taking targeting peptide insertions into account) to a VP1 capsid polypeptide of SEQ ID NO:18 or the VP2 or VP3 portion thereof.
214. The capsid polypeptide of any one of embodiments 1 to 83, which comprises an amino acid sequence having at least 75% sequence identity ((a) calculated taking targeting peptide insertions into account or (b) calculated without taking targeting peptide insertions into account) to a VP1 capsid polypeptide of SEQ ID NO:18 or the VP2 or VP3 portion thereof.
215. The capsid polypeptide of any one of embodiments 1 to 83, which comprises an amino acid sequence having at least 80% sequence identity ((a) calculated taking targeting peptide insertions into account or (b) calculated without taking targeting peptide insertions into account) to a VP1 capsid polypeptide of SEQ ID NO:18 or the VP2 or VP3 portion thereof.
216. The capsid polypeptide of any one of embodiments 1 to 83, which comprises an amino acid sequence having at least 85% sequence identity ((a) calculated taking targeting peptide insertions into account or (b) calculated without taking targeting peptide insertions into account) to a VP1 capsid polypeptide of SEQ ID NO:18 or the VP2 or VP3 portion thereof.
217. The capsid polypeptide of any one of embodiments 1 to 83, which comprises an amino acid sequence having at least 90% sequence identity ((a) calculated taking targeting peptide insertions into account or (b) calculated without taking targeting peptide insertions into account) to a VP1 capsid polypeptide of SEQ ID NO:18 or the VP2 or VP3 portion thereof.
218. The capsid polypeptide of any one of embodiments 1 to 83, which comprises an amino acid sequence having at least 91% sequence identity ((a) calculated taking targeting peptide insertions into account or (b) calculated without taking targeting peptide insertions into account) to a VP1 capsid polypeptide of SEQ ID NO:18 or the VP2 or VP3 portion thereof.
219. The capsid polypeptide of any one of embodiments 1 to 83, which comprises an amino acid sequence having at least 92% sequence identity ((a) calculated taking targeting peptide insertions into account or (b) calculated without taking targeting peptide insertions into account) to a VP1 capsid polypeptide of SEQ ID NO:18 or the VP2 or VP3 portion thereof.
220. The capsid polypeptide of any one of embodiments 1 to 83, which comprises an amino acid sequence having at least 93% sequence identity ((a) calculated taking targeting peptide insertions into account or (b) calculated without taking targeting peptide insertions into account) to a VP1 capsid polypeptide of SEQ ID NO:18 or the VP2 or VP3 portion thereof.
221. The capsid polypeptide of any one of embodiments 1 to 83, which comprises an amino acid sequence having at least 94% sequence identity ((a) calculated taking targeting peptide insertions into account or (b) calculated without taking targeting peptide insertions into account) to a VP1 capsid polypeptide of SEQ ID NO:18 or the VP2 or VP3 portion thereof.
222. The capsid polypeptide of any one of embodiments 1 to 83, which comprises an amino acid sequence having at least 95% sequence identity ((a) calculated taking targeting peptide insertions into account or (b) calculated without taking targeting peptide insertions into account) to a VP1 capsid polypeptide of SEQ ID NO:18 or the VP2 or VP3 portion thereof.
223. The capsid polypeptide of any one of embodiments 1 to 83, which comprises an amino acid sequence having at least 96% sequence identity ((a) calculated taking targeting peptide insertions into account or (b) calculated without taking targeting peptide insertions into account) to a VP1 capsid polypeptide of SEQ ID NO:18 or the VP2 or VP3 portion thereof.
224. The capsid polypeptide of any one of embodiments 1 to 83, which comprises an amino acid sequence having at least 97% sequence identity ((a) calculated taking targeting peptide insertions into account or (b) calculated without taking targeting peptide insertions into account) to a VP1 capsid polypeptide of SEQ ID NO:18 or the VP2 or VP3 portion thereof.
225. The capsid polypeptide of any one of embodiments 1 to 83, which comprises an amino acid sequence having at least 98% sequence identity ((a) calculated taking targeting peptide insertions into account or (b) calculated without taking targeting peptide insertions into account) to a VP1 capsid polypeptide of SEQ ID NO:18 or the VP2 or VP3 portion thereof.
226. The capsid polypeptide of any one of embodiments 1 to 83, which comprises an amino acid sequence having at least 99% sequence identity ((a) calculated taking targeting peptide insertions into account or (b) calculated without taking targeting peptide insertions into account) to a VP1 capsid polypeptide of SEQ ID NO:18 or the VP2 or VP3 portion thereof.
227. A capsid polypeptide, which is optionally a capsid polypeptide according to any one of embodiments 1 to 83, which comprises the amino acid sequence of a VP1 capsid polypeptide of SEQ ID NO:18 or the VP2 or VP3 portion thereof.
228. The capsid polypeptide of any one of embodiments 1 to 83, which comprises an amino acid sequence having at least 70% sequence identity ((a) calculated taking targeting peptide insertions into account or (b) calculated without taking targeting peptide insertions into account) to a VP1 capsid polypeptide of any one of SEQ ID NOS:300-445 or the VP2 or VP3 portion thereof.
229. The capsid polypeptide of any one of embodiments 1 to 83, which comprises an amino acid sequence having at least 75% sequence identity ((a) calculated taking targeting peptide insertions into account or (b) calculated without taking targeting peptide insertions into account) to a VP1 capsid polypeptide of any one of SEQ ID NOS:300-445 or the VP2 or VP3 portion thereof.
230. The capsid polypeptide of any one of embodiments 1 to 83, which comprises an amino acid sequence having at least 80% sequence identity ((a) calculated taking targeting peptide insertions into account or (b) calculated without taking targeting peptide insertions into account) to a VP1 capsid polypeptide of any one of SEQ ID NOS:300-445 or the VP2 or VP3 portion thereof.
231. The capsid polypeptide of any one of embodiments 1 to 83, which comprises an amino acid sequence having at least 85% sequence identity ((a) calculated taking targeting peptide insertions into account or (b) calculated without taking targeting peptide insertions into account) to a VP1 capsid polypeptide of any one of SEQ ID NOS:300-445 or the VP2 or VP3 portion thereof.
232. The capsid polypeptide of any one of embodiments 1 to 83, which comprises an amino acid sequence having at least 90% sequence identity ((a) calculated taking targeting peptide insertions into account or (b) calculated without taking targeting peptide insertions into account) to a VP1 capsid polypeptide of any one of SEQ ID NOS:300-445 or the VP2 or VP3 portion thereof.
233. The capsid polypeptide of any one of embodiments 1 to 83, which comprises an amino acid sequence having at least 91% sequence identity ((a) calculated taking targeting peptide insertions into account or (b) calculated without taking targeting peptide insertions into account) to a VP1 capsid polypeptide of any one of SEQ ID NOS:300-445 or the VP2 or VP3 portion thereof.
234. The capsid polypeptide of any one of embodiments 1 to 83, which comprises an amino acid sequence having at least 92% sequence identity ((a) calculated taking targeting peptide insertions into account or (b) calculated without taking targeting peptide insertions into account) to a VP1 capsid polypeptide of any one of SEQ ID NOS:300-445 or the VP2 or VP3 portion thereof.
235. The capsid polypeptide of any one of embodiments 1 to 83, which comprises an amino acid sequence having at least 93% sequence identity ((a) calculated taking targeting peptide insertions into account or (b) calculated without taking targeting peptide insertions into account) to a VP1 capsid polypeptide of any one of SEQ ID NOS:300-445 or the VP2 or VP3 portion thereof.
236. The capsid polypeptide of any one of embodiments 1 to 83, which comprises an amino acid sequence having at least 94% sequence identity ((a) calculated taking targeting peptide insertions into account or (b) calculated without taking targeting peptide insertions into account) to a VP1 capsid polypeptide of any one of SEQ ID NOS:300-445 or the VP2 or VP3 portion thereof.
237. The capsid polypeptide of any one of embodiments 1 to 83, which comprises an amino acid sequence having at least 95% sequence identity ((a) calculated taking targeting peptide insertions into account or (b) calculated without taking targeting peptide insertions into account) to a VP1 capsid polypeptide of any one of SEQ ID NOS:300-445 or the VP2 or VP3 portion thereof.
238. The capsid polypeptide of any one of embodiments 1 to 83, which comprises an amino acid sequence having at least 96% sequence identity ((a) calculated taking targeting peptide insertions into account or (b) calculated without taking targeting peptide insertions into account) to a VP1 capsid polypeptide of any one of SEQ ID NOS:300-445 or the VP2 or VP3 portion thereof.
239. The capsid polypeptide of any one of embodiments 1 to 83, which comprises an amino acid sequence having at least 97% sequence identity ((a) calculated taking targeting peptide insertions into account or (b) calculated without taking targeting peptide insertions into account) to a VP1 capsid polypeptide of any one of SEQ ID NOS:300-445 or the VP2 or VP3 portion thereof.
240. The capsid polypeptide of any one of embodiments 1 to 83, which comprises an amino acid sequence having at least 98% sequence identity ((a) calculated taking targeting peptide insertions into account or (b) calculated without taking targeting peptide insertions into account) to a VP1 capsid polypeptide of any one of SEQ ID NOS:300-445 or the VP2 or VP3 portion thereof.
241. The capsid polypeptide of any one of embodiments 1 to 83, which comprises an amino acid sequence having at least 99% sequence identity ((a) calculated taking targeting peptide insertions into account or (b) calculated without taking targeting peptide insertions into account) to a VP1 capsid polypeptide of any one of SEQ ID NOS:300-445 or the VP2 or VP3 portion thereof.
242. A capsid polypeptide, which is optionally a capsid polypeptide according to any one of embodiments 1 to 83, which comprises the amino acid sequence of a VP1 capsid polypeptide of any one of SEQ ID NOS:300-445 or the VP2 or VP3 portion thereof.
243. A capsid polypeptide which:
   (a) comprises the mutations in the mutation set present in the amino acid sequence of SEQ ID NO:12 as compared to the amino acid sequence of SEQ ID NO:7; and
   (b) comprises an amino acid sequence with an edit distance of 15 or less to the amino acid sequence of SEQ ID NO:12 (or to the VP2 or VP3 portion thereof).
244. The capsid polypeptide of embodiment 243, comprising an amino acid sequence with an edit distance of 14 or less to the amino acid sequence of SEQ ID NO:12 (or to the VP2 or VP3 portion thereof).
245. The capsid polypeptide of embodiment 243, comprising an amino acid sequence with an edit distance of 13 or less to the amino acid sequence of SEQ ID NO:12 (or to the VP2 or VP3 portion thereof).
246. The capsid polypeptide of embodiment 243, comprising an amino acid sequence with an edit distance of 12 or less to the amino acid sequence of SEQ ID NO:12 (or to the VP2 or VP3 portion thereof).
247. The capsid polypeptide of embodiment 243, comprising an amino acid sequence with an edit distance of 11 or less to the amino acid sequence of SEQ ID NO:12 (or to the VP2 or VP3 portion thereof).
248. The capsid polypeptide of embodiment 243, comprising an amino acid sequence with an edit distance of 10 or less to the amino acid sequence of SEQ ID NO:12 (or to the VP2 or VP3 portion thereof).
249. The capsid polypeptide of embodiment 243, comprising an amino acid sequence with an edit distance of 9 or less to the amino acid sequence of SEQ ID NO:12 (or to the VP2 or VP3 portion thereof).
250. The capsid polypeptide of embodiment 243, comprising an amino acid sequence with an edit distance of 8 or less to the amino acid sequence of SEQ ID NO:12 (or to the VP2 or VP3 portion thereof).
251. The capsid polypeptide of embodiment 243, comprising an amino acid sequence with an edit distance of 7 or less to the amino acid sequence of SEQ ID NO:12 (or to the VP2 or VP3 portion thereof).
252. The capsid polypeptide of embodiment 243, comprising an amino acid sequence with an edit distance of 6 or less to the amino acid sequence of SEQ ID NO:12 (or to the VP2 or VP3 portion thereof).
253. The capsid polypeptide of embodiment 243, comprising an amino acid sequence with an edit distance of 5 or less to the amino acid sequence of SEQ ID NO:12 (or to the VP2 or VP3 portion thereof).
254. The capsid polypeptide of embodiment 243, comprising an amino acid sequence with an edit distance of 4 or less to the amino acid sequence of SEQ ID NO:12 (or to the VP2 or VP3 portion thereof).
255. The capsid polypeptide of embodiment 243, comprising an amino acid sequence with an edit distance of 3 or less to the amino acid sequence of SEQ ID NO:12 (or to the VP2 or VP3 portion thereof).
256. The capsid polypeptide of embodiment 243, comprising an amino acid sequence with an edit distance of 2 or less to the amino acid sequence of SEQ ID NO:12 (or to the VP2 or VP3 portion thereof).
257. The capsid polypeptide of embodiment 243, comprising an amino acid sequence with an edit distance of 1 or less to the amino acid sequence of SEQ ID NO:12 (or to the VP2 or VP3 portion thereof).
258. A capsid polypeptide which:
   (a) comprises the mutations in the mutation set present in the amino acid sequence of SEQ ID NO:14 as compared to the amino acid sequence of SEQ ID NO:7; and
   (b) comprises an amino acid sequence with an edit distance of 15 or less to the amino acid sequence of SEQ ID NO:14 (or to the VP2 or VP3 portion thereof).
259. The capsid polypeptide of embodiment 258, comprising an amino acid sequence with an edit distance of 14 or less to the amino acid sequence of SEQ ID NO:14 (or to the VP2 or VP3 portion thereof).
260. The capsid polypeptide of embodiment 258, comprising an amino acid sequence with an edit distance of 13 or less to the amino acid sequence of SEQ ID NO:14 (or to the VP2 or VP3 portion thereof).
261. The capsid polypeptide of embodiment 258, comprising an amino acid sequence with an edit distance of 12 or less to the amino acid sequence of SEQ ID NO:14 (or to the VP2 or VP3 portion thereof).
262. The capsid polypeptide of embodiment 258, comprising an amino acid sequence with an edit distance of 11 or less to the amino acid sequence of SEQ ID NO:14 (or to the VP2 or VP3 portion thereof).
263. The capsid polypeptide of embodiment 258, comprising an amino acid sequence with an edit distance of 10 or less to the amino acid sequence of SEQ ID NO:14 (or to the VP2 or VP3 portion thereof).
264. The capsid polypeptide of embodiment 258, comprising an amino acid sequence with an edit distance of 9 or less to the amino acid sequence of SEQ ID NO:14 (or to the VP2 or VP3 portion thereof).
265. The capsid polypeptide of embodiment 258, comprising an amino acid sequence with an edit distance of 8 or less to the amino acid sequence of SEQ ID NO:14 (or to the VP2 or VP3 portion thereof).
266. The capsid polypeptide of embodiment 258, comprising an amino acid sequence with an edit distance of 7 or less to the amino acid sequence of SEQ ID NO:14 (or to the VP2 or VP3 portion thereof).
267. The capsid polypeptide of embodiment 258, comprising an amino acid sequence with an edit distance of 6 or less to the amino acid sequence of SEQ ID NO:14 (or to the VP2 or VP3 portion thereof).
268. The capsid polypeptide of embodiment 258, comprising an amino acid sequence with an edit distance of 5 or less to the amino acid sequence of SEQ ID NO:14 (or to the VP2 or VP3 portion thereof).
269. The capsid polypeptide of embodiment 258, comprising an amino acid sequence with an edit distance of 4 or less to the amino acid sequence of SEQ ID NO:14 (or to the VP2 or VP3 portion thereof).
270. The capsid polypeptide of embodiment 258, comprising an amino acid sequence with an edit distance of 3 or less to the amino acid sequence of SEQ ID NO:14 (or to the VP2 or VP3 portion thereof).
271. The capsid polypeptide of embodiment 258, comprising an amino acid sequence with an edit distance of 2 or less to the amino acid sequence of SEQ ID NO:14 (or to the VP2 or VP3 portion thereof).
272. The capsid polypeptide of embodiment 258, comprising an amino acid sequence with an edit distance of 1 or less to the amino acid sequence of SEQ ID NO:14 (or to the VP2 or VP3 portion thereof).
273. A capsid polypeptide which:
   (a) comprises the mutations in the mutation set present in the amino acid sequence of SEQ ID NO:16 as compared to the amino acid sequence of SEQ ID NO:7; and
   (b) comprises an amino acid sequence with an edit distance of 15 or less to the amino acid sequence of SEQ ID NO:16 (or to the VP2 or VP3 portion thereof).
274. The capsid polypeptide of embodiment 273, comprising an amino acid sequence with an edit distance of 14 or less to the amino acid sequence of SEQ ID NO:16 (or to the VP2 or VP3 portion thereof).
275. The capsid polypeptide of embodiment 273, comprising an amino acid sequence with an edit distance of 13 or less to the amino acid sequence of SEQ ID NO:16 (or to the VP2 or VP3 portion thereof).
276. The capsid polypeptide of embodiment 273, comprising an amino acid sequence with an edit distance of 12 or less to the amino acid sequence of SEQ ID NO:16 (or to the VP2 or VP3 portion thereof).
277. The capsid polypeptide of embodiment 273, comprising an amino acid sequence with an edit distance of 11 or less to the amino acid sequence of SEQ ID NO:16 (or to the VP2 or VP3 portion thereof).
278. The capsid polypeptide of embodiment 273, comprising an amino acid sequence with an edit distance of 10 or less to the amino acid sequence of SEQ ID NO:16 (or to the VP2 or VP3 portion thereof).
279. The capsid polypeptide of embodiment 273, comprising an amino acid sequence with an edit distance of 9 or less to the amino acid sequence of SEQ ID NO:16 (or to the VP2 or VP3 portion thereof).
280. The capsid polypeptide of embodiment 273, comprising an amino acid sequence with an edit distance of 8 or less to the amino acid sequence of SEQ ID NO:16 (or to the VP2 or VP3 portion thereof).
281. The capsid polypeptide of embodiment 273, comprising an amino acid sequence with an edit distance of 7 or less to the amino acid sequence of SEQ ID NO:16 (or to the VP2 or VP3 portion thereof).
282. The capsid polypeptide of embodiment 273, comprising an amino acid sequence with an edit distance of 6 or less to the amino acid sequence of SEQ ID NO:16 (or to the VP2 or VP3 portion thereof).
283. The capsid polypeptide of embodiment 273, comprising an amino acid sequence with an edit distance of 5 or less to the amino acid sequence of SEQ ID NO:16 (or to the VP2 or VP3 portion thereof).
284. The capsid polypeptide of embodiment 273, comprising an amino acid sequence with an edit distance of 4 or less to the amino acid sequence of SEQ ID NO:16 (or to the VP2 or VP3 portion thereof).
285. The capsid polypeptide of embodiment 273, comprising an amino acid sequence with an edit distance of 3 or less to the amino acid sequence of SEQ ID NO:16 (or to the VP2 or VP3 portion thereof).
286. The capsid polypeptide of embodiment 273, comprising an amino acid sequence with an edit distance of 2 or less to the amino acid sequence of SEQ ID NO:16 (or to the VP2 or VP3 portion thereof).
287. The capsid polypeptide of embodiment 273, comprising an amino acid sequence with an edit distance of 1 or less to the amino acid sequence of SEQ ID NO:16 (or to the VP2 or VP3 portion thereof).
288. A capsid polypeptide which:
   (a) comprises the mutations in the mutation set present in the amino acid sequence of SEQ ID NO:18 as compared to the amino acid sequence of SEQ ID NO:7; and
   (b) comprises an amino acid sequence with an edit distance of 15 or less to the amino acid sequence of SEQ ID NO:18 (or to the VP2 or VP3 portion thereof).
289. The capsid polypeptide of embodiment 288, comprising an amino acid sequence with an edit distance of 14 or less to the amino acid sequence of SEQ ID NO:18 (or to the VP2 or VP3 portion thereof).
290. The capsid polypeptide of embodiment 288, comprising an amino acid sequence with an edit distance of 13 or less to the amino acid sequence of SEQ ID NO:18 (or to the VP2 or VP3 portion thereof).
291. The capsid polypeptide of embodiment 288, comprising an amino acid sequence with an edit distance of 12 or less to the amino acid sequence of SEQ ID NO:18 (or to the VP2 or VP3 portion thereof).
292. The capsid polypeptide of embodiment 288, comprising an amino acid sequence with an edit distance of 11 or less to the amino acid sequence of SEQ ID NO:18 (or to the VP2 or VP3 portion thereof).
293. The capsid polypeptide of embodiment 288, comprising an amino acid sequence with an edit distance of 10 or less to the amino acid sequence of SEQ ID NO:18 (or to the VP2 or VP3 portion thereof).
294. The capsid polypeptide of embodiment 288, comprising an amino acid sequence with an edit distance of 9 or less to the amino acid sequence of SEQ ID NO:18 (or to the VP2 or VP3 portion thereof).
295. The capsid polypeptide of embodiment 288, comprising an amino acid sequence with an edit distance of 8 or less to the amino acid sequence of SEQ ID NO:18 (or to the VP2 or VP3 portion thereof).
296. The capsid polypeptide of embodiment 288, comprising an amino acid sequence with an edit distance of 7 or less to the amino acid sequence of SEQ ID NO:18 (or to the VP2 or VP3 portion thereof).
297. The capsid polypeptide of embodiment 288, comprising an amino acid sequence with an edit distance of 6 or less to the amino acid sequence of SEQ ID NO:18 (or to the VP2 or VP3 portion thereof).
298. The capsid polypeptide of embodiment 288, comprising an amino acid sequence with an edit distance of 5 or less to the amino acid sequence of SEQ ID NO:18 (or to the VP2 or VP3 portion thereof).
299. The capsid polypeptide of embodiment 288, comprising an amino acid sequence with an edit distance of 4 or less to the amino acid sequence of SEQ ID NO:18 (or to the VP2 or VP3 portion thereof).
300. The capsid polypeptide of embodiment 288, comprising an amino acid sequence with an edit distance of 3 or less to the amino acid sequence of SEQ ID NO:18 (or to the VP2 or VP3 portion thereof).
301. The capsid polypeptide of embodiment 288, comprising an amino acid sequence with an edit distance of 2 or less to the amino acid sequence of SEQ ID NO:18 (or to the VP2 or VP3 portion thereof).
302. The capsid polypeptide of embodiment 288, comprising an amino acid sequence with an edit distance of 1 or less to the amino acid sequence of SEQ ID NO:18 (or to the VP2 or VP3 portion thereof).
303. A capsid polypeptide which:
   (a) comprises the mutations in the mutation set present in an amino acid sequence selected from any one of SEQ ID NOS:300-445 as compared to the amino acid sequence of SEQ ID NO:7; and
   (b) comprises an amino acid sequence with an edit distance of 15 or less to the selected amino acid sequence (or to the VP2 or VP3 portion thereof).
304. The capsid polypeptide of embodiment 303, comprising an amino acid sequence with an edit distance of 14 or less to the selected amino acid sequence (or to the VP2 or VP3 portion thereof).
305. The capsid polypeptide of embodiment 303, comprising an amino acid sequence with an edit distance of 13 or less to the selected amino acid sequence (or to the VP2 or VP3 portion thereof).
306. The capsid polypeptide of embodiment 303, comprising an amino acid sequence with an edit distance of 12 or less to the selected amino acid sequence (or to the VP2 or VP3 portion thereof).
307. The capsid polypeptide of embodiment 303, comprising an amino acid sequence with an edit distance of 11 or less to the selected amino acid sequence (or to the VP2 or VP3 portion thereof).
308. The capsid polypeptide of embodiment 303, comprising an amino acid sequence with an edit distance of 10 or less to the selected amino acid sequence (or to the VP2 or VP3 portion thereof).
309. The capsid polypeptide of embodiment 303, comprising an amino acid sequence with an edit distance of 9 or less to the selected amino acid sequence (or to the VP2 or VP3 portion thereof).
310. The capsid polypeptide of embodiment 303, comprising an amino acid sequence with an edit distance of 8 or less to the selected amino acid sequence (or to the VP2 or VP3 portion thereof).
311. The capsid polypeptide of embodiment 303, comprising an amino acid sequence with an edit distance of 7 or less to the selected amino acid sequence (or to the VP2 or VP3 portion thereof).
312. The capsid polypeptide of embodiment 303, comprising an amino acid sequence with an edit distance of 6 or less to the selected amino acid sequence (or to the VP2 or VP3 portion thereof).
313. The capsid polypeptide of embodiment 303, comprising an amino acid sequence with an edit distance of 5 or less to the selected amino acid sequence (or to the VP2 or VP3 portion thereof).
314. The capsid polypeptide of embodiment 303, comprising an amino acid sequence with an edit distance of 4 or less to the selected amino acid sequence (or to the VP2 or VP3 portion thereof).
315. The capsid polypeptide of embodiment 303, comprising an amino acid sequence with an edit distance of 3 or less to the selected amino acid sequence (or to the VP2 or VP3 portion thereof).
316. The capsid polypeptide of embodiment 303, comprising an amino acid sequence with an edit distance of 2 or less to the selected amino acid sequence (or to the VP2 or VP3 portion thereof).
317. The capsid polypeptide of embodiment 303, comprising an amino acid sequence with an edit distance of 1 or less to the selected amino acid sequence (or to the VP2 or VP3 portion thereof).
318. The capsid polypeptide of any one of embodiments 1 to 317, whose sequence has an edit distance of (a) 12 or lower, (b) 11 or lower, or (c) 10 or lower to a VP1 capsid polypeptide of SEQ ID NO:7 or the VP2 or VP3 portion thereof.
319. The capsid polypeptide of any one of embodiments 1 to 318, whose sequence has an edit distance of (a) 12 or lower, (b) 11 or lower, or (c) 10 or lower to:
   (a) a VP1 capsid polypeptide of SEQ ID NO:12;
   (b) a VP2 capsid polypeptide corresponding to the VP2 portion of SEQ ID NO:12; or
   (c) a VP3 capsid polypeptide corresponding to the VP3 portion of SEQ ID NO:12.
320. The capsid polypeptide of any one of embodiments 1 to 318, whose sequence has an edit distance of (a) 12 or lower, (b) 11 or lower, or (c) 10 or lower to:
   (a) a VP1 capsid polypeptide of SEQ ID NO:14;
   (b) a VP2 capsid polypeptide corresponding to the VP2 portion of SEQ ID NO:14; or
   (c) a VP3 capsid polypeptide corresponding to the VP3 portion of SEQ ID NO:14.
321. The capsid polypeptide of any one of embodiments 1 to 318, whose sequence has an edit distance of (a) 12 or lower, (b) 11 or lower, or (c) 10 or lower to:
   (a) a VP1 capsid polypeptide of SEQ ID NO:16;
   (b) a VP2 capsid polypeptide corresponding to the VP2 portion of SEQ ID NO:16; or
   (c) a VP3 capsid polypeptide corresponding to the VP3 portion of SEQ ID NO:16.
322. The capsid polypeptide of any one of embodiments 1 to 318, whose sequence has an edit distance of (a) 12 or lower, (b) 11 or lower, or (c) 10 or lower to:
   (a) a VP1 capsid polypeptide of SEQ ID NO:18;
   (b) a VP2 capsid polypeptide corresponding to the VP2 portion of SEQ ID NO:18; or
   (c) a VP3 capsid polypeptide corresponding to the VP3 portion of SEQ ID NO:18.
323. The capsid polypeptide of any one of embodiments 1 to 318, whose sequence has an edit distance of (a) 12 or lower, (b) 11 or lower, or (c) 10 or lower to:
   (a) a VP1 capsid polypeptide of any one of SEQ ID NOS:300-445;
   (b) a VP2 capsid polypeptide corresponding to the VP2 portion of any one of SEQ ID NOS:300-445; or
   (c) a VP3 capsid polypeptide corresponding to the VP3 portion of any one of SEQ ID NOS:300-445.
324. The capsid polypeptide of any one of embodiments 1 to 323, which is a VP1 capsid polypeptide.
325. The capsid polypeptide of any one of embodiments 1 to 323, which is a VP2 capsid polypeptide.
326. The capsid polypeptide of any one of embodiments 1 to 323, which is a VP3 capsid polypeptide.
327. A nucleic acid molecule encoding a capsid polypeptide of any one of embodiments 1 to 326.
328. The nucleic acid molecule of embodiment 327, wherein the nucleic acid molecule comprises a nucleotide sequence having at least 70% sequence identity to the nucleotide sequence of SEQ ID NO:13 or a fragment thereof (e.g., a VP1-encoding, a VP2-encoding or a VP3-encoding fragment thereof).
329. The nucleic acid molecule of embodiment 327, wherein the nucleic acid molecule comprises a nucleotide sequence having at least 80% sequence identity to the nucleotide sequence of SEQ ID NO:13 or a fragment thereof (e.g., a VP1-encoding, a VP2-encoding or a VP3-encoding fragment thereof).
330. The nucleic acid molecule of embodiment 327, wherein the nucleic acid molecule comprises a nucleotide sequence having at least 85% sequence identity to the nucleotide sequence of SEQ ID NO:13 or a fragment thereof (e.g., a VP1-encoding, a VP2-encoding or a VP3-encoding fragment thereof).
331. The nucleic acid molecule of embodiment 327, wherein the nucleic acid molecule comprises a nucleotide sequence having at least 90% sequence identity to the nucleotide sequence of SEQ ID NO:13 or a fragment thereof (e.g., a VP1-encoding, a VP2-encoding or a VP3-encoding fragment thereof).
332. The nucleic acid molecule of embodiment 327, wherein the nucleic acid molecule comprises a nucleotide sequence having at least 91% sequence identity to the nucleotide sequence of SEQ ID NO:13 or a fragment thereof (e.g., a VP1-encoding, a VP2-encoding or a VP3-encoding fragment thereof).
333. The nucleic acid molecule of embodiment 327, wherein the nucleic acid molecule comprises a nucleotide sequence having at least 92% sequence identity to the nucleotide sequence of SEQ ID NO:13 or a fragment thereof (e.g., a VP1-encoding, a VP2-encoding or a VP3-encoding fragment thereof).
334. The nucleic acid molecule of embodiment 327, wherein the nucleic acid molecule comprises a nucleotide sequence having at least 93% sequence identity to the nucleotide sequence of SEQ ID NO:13 or a fragment thereof (e.g., a VP1-encoding, a VP2-encoding or a VP3-encoding fragment thereof).
335. The nucleic acid molecule of embodiment 327, wherein the nucleic acid molecule comprises a nucleotide sequence having at least 94% sequence identity to the nucleotide sequence of SEQ ID NO:13 or a fragment thereof (e.g., a VP1-encoding, a VP2-encoding or a VP3-encoding fragment thereof).
336. The nucleic acid molecule of embodiment 327, wherein the nucleic acid molecule comprises a nucleotide sequence having at least 95% sequence identity to the nucleotide sequence of SEQ ID NO:13 or a fragment thereof (e.g., a VP1-encoding, a VP2-encoding or a VP3-encoding fragment thereof).
337. The nucleic acid molecule of embodiment 327, wherein the nucleic acid molecule comprises a nucleotide sequence having at least 96% sequence identity to the nucleotide sequence of SEQ ID NO:13 or a fragment thereof (e.g., a VP1-encoding, a VP2-encoding or a VP3-encoding fragment thereof).
338. The nucleic acid molecule of embodiment 327, wherein the nucleic acid molecule comprises a nucleotide sequence having at least 97% sequence identity to the nucleotide sequence of SEQ ID NO:13 or a fragment thereof (e.g., a VP1-encoding, a VP2-encoding or a VP3-encoding fragment thereof).
339. The nucleic acid molecule of embodiment 327, wherein the nucleic acid molecule comprises a nucleotide sequence having at least 98% sequence identity to the nucleotide sequence of SEQ ID NO:13 or a fragment thereof (e.g., a VP1-encoding, a VP2-encoding or a VP3-encoding fragment thereof).
340. The nucleic acid molecule of embodiment 327, wherein the nucleic acid molecule comprises a nucleotide sequence having at least 99% sequence identity to the nucleotide sequence of SEQ ID NO:13 or a fragment thereof (e.g., a VP1-encoding, a VP2-encoding or a VP3-encoding fragment thereof).
341. The nucleic acid molecule of embodiment 327, wherein the nucleic acid molecule comprises the nucleotide sequence of SEQ ID NO:13 or a fragment thereof (e.g., a VP1-encoding, a VP2-encoding or a VP3-encoding fragment thereof).
342. The nucleic acid molecule of any one of embodiments 327 to 341, wherein the nucleic acid molecule comprises the nucleotide sequence of SEQ ID NO:13.
343. The nucleic acid molecule of any one of embodiments 327 to 341, wherein the nucleic acid molecule comprises a fragment of the nucleotide sequence of SEQ ID NO:13 that encodes a VP2 capsid polypeptide.
344. The nucleic acid molecule of any one of embodiments 327 to 341, wherein the nucleic acid molecule comprises a fragment of the nucleotide sequence of SEQ ID NO:13 that encodes a VP3 capsid polypeptide.
345. The nucleic acid molecule of embodiment 327, wherein the nucleic acid molecule comprises a nucleotide sequence having at least 70% sequence identity to the nucleotide sequence of SEQ ID NO:15 or a fragment thereof (e.g., a VP1-encoding, a VP2-encoding or a VP3-encoding fragment thereof).
346. The nucleic acid molecule of embodiment 327, wherein the nucleic acid molecule comprises a nucleotide sequence having at least 80% sequence identity to the nucleotide sequence of SEQ ID NO:15 or a fragment thereof (e.g., a VP1-encoding, a VP2-encoding or a VP3-encoding fragment thereof).
347. The nucleic acid molecule of embodiment 327, wherein the nucleic acid molecule comprises a nucleotide sequence having at least 85% sequence identity to the nucleotide sequence of SEQ ID NO:15 or a fragment thereof (e.g., a VP1-encoding, a VP2-encoding or a VP3-encoding fragment thereof).
348. The nucleic acid molecule of embodiment 327, wherein the nucleic acid molecule comprises a nucleotide sequence having at least 90% sequence identity to the nucleotide sequence of SEQ ID NO:15 or a fragment thereof (e.g., a VP1-encoding, a VP2-encoding or a VP3-encoding fragment thereof).
349. The nucleic acid molecule of embodiment 327, wherein the nucleic acid molecule comprises a nucleotide sequence having at least 91% sequence identity to the nucleotide sequence of SEQ ID NO:15 or a fragment thereof (e.g., a VP1-encoding, a VP2-encoding or a VP3-encoding fragment thereof).
350. The nucleic acid molecule of embodiment 327, wherein the nucleic acid molecule comprises a nucleotide sequence having at least 92% sequence identity to the nucleotide sequence of SEQ ID NO:15 or a fragment thereof (e.g., a VP1-encoding, a VP2-encoding or a VP3-encoding fragment thereof).
351. The nucleic acid molecule of embodiment 327, wherein the nucleic acid molecule comprises a nucleotide sequence having at least 93% sequence identity to the nucleotide sequence of SEQ ID NO:15 or a fragment thereof (e.g., a VP1-encoding, a VP2-encoding or a VP3-encoding fragment thereof).
352. The nucleic acid molecule of embodiment 327, wherein the nucleic acid molecule comprises a nucleotide sequence having at least 94% sequence identity to the nucleotide sequence of SEQ ID NO:15 or a fragment thereof (e.g., a VP1-encoding, a VP2-encoding or a VP3-encoding fragment thereof).
353. The nucleic acid molecule of embodiment 327, wherein the nucleic acid molecule comprises a nucleotide sequence having at least 95% sequence identity to the nucleotide sequence of SEQ ID NO:15 or a fragment thereof (e.g., a VP1-encoding, a VP2-encoding or a VP3-encoding fragment thereof).
354. The nucleic acid molecule of embodiment 327, wherein the nucleic acid molecule comprises a nucleotide sequence having at least 96% sequence identity to the nucleotide sequence of SEQ ID NO:15 or a fragment thereof (e.g., a VP1-encoding, a VP2-encoding or a VP3-encoding fragment thereof).
355. The nucleic acid molecule of embodiment 327, wherein the nucleic acid molecule comprises a nucleotide sequence having at least 97% sequence identity to the nucleotide sequence of SEQ ID NO:15 or a fragment thereof (e.g., a VP1-encoding, a VP2-encoding or a VP3-encoding fragment thereof).
356. The nucleic acid molecule of embodiment 327, wherein the nucleic acid molecule comprises a nucleotide sequence having at least 98% sequence identity to the nucleotide sequence of SEQ ID NO:15 or a fragment thereof (e.g., a VP1-encoding, a VP2-encoding or a VP3-encoding fragment thereof).
357. The nucleic acid molecule of embodiment 327, wherein the nucleic acid molecule comprises a nucleotide sequence having at least 99% sequence identity to the nucleotide sequence of SEQ ID NO:15 or a fragment thereof (e.g., a VP1-encoding, a VP2-encoding or a VP3-encoding fragment thereof).
358. The nucleic acid molecule of embodiment 327, wherein the nucleic acid molecule comprises the nucleotide sequence of SEQ ID NO:15 or a fragment thereof (e.g., a VP1-encoding, a VP2-encoding or a VP3-encoding fragment thereof). 359. The nucleic acid molecule of any one of embodiments 345 to 358, wherein the nucleic acid molecule comprises the nucleotide sequence of SEQ ID NO:15.
360. The nucleic acid molecule of any one of embodiments 345 to 358, wherein the nucleic acid molecule comprises a fragment of the nucleotide sequence of SEQ ID NO:15 that encodes a VP2 capsid polypeptide.
361. The nucleic acid molecule of any one of embodiments 345 to 358, wherein the nucleic acid molecule comprises a fragment of the nucleotide sequence of SEQ ID NO:15 that encodes a VP3 capsid polypeptide.
362. The nucleic acid molecule of embodiment 327, wherein the nucleic acid molecule comprises a nucleotide sequence having at least 70% sequence identity to the nucleotide sequence of SEQ ID NO:17 or a fragment thereof (e.g., a VP1-encoding, a VP2-encoding or a VP3-encoding fragment thereof).
363. The nucleic acid molecule of embodiment 327, wherein the nucleic acid molecule comprises a nucleotide sequence having at least 80% sequence identity to the nucleotide sequence of SEQ ID NO:17 or a fragment thereof (e.g., a VP1-encoding, a VP2-encoding or a VP3-encoding fragment thereof).
364. The nucleic acid molecule of embodiment 327, wherein the nucleic acid molecule comprises a nucleotide sequence having at least 85% sequence identity to the nucleotide sequence of SEQ ID NO:17 or a fragment thereof (e.g., a VP1-encoding, a VP2-encoding or a VP3-encoding fragment thereof).
365. The nucleic acid molecule of embodiment 327, wherein the nucleic acid molecule comprises a nucleotide sequence having at least 90% sequence identity to the nucleotide sequence of SEQ ID NO:17 or a fragment thereof (e.g., a VP1-encoding, a VP2-encoding or a VP3-encoding fragment thereof).
366. The nucleic acid molecule of embodiment 327, wherein the nucleic acid molecule comprises a nucleotide sequence having at least 91% sequence identity to the nucleotide sequence of SEQ ID NO:17 or a fragment thereof (e.g., a VP1-encoding, a VP2-encoding or a VP3-encoding fragment thereof).
367. The nucleic acid molecule of embodiment 327, wherein the nucleic acid molecule comprises a nucleotide sequence having at least 92% sequence identity to the nucleotide sequence of SEQ ID NO:17 or a fragment thereof (e.g., a VP1-encoding, a VP2-encoding or a VP3-encoding fragment thereof).
368. The nucleic acid molecule of embodiment 327, wherein the nucleic acid molecule comprises a nucleotide sequence having at least 93% sequence identity to the nucleotide sequence of SEQ ID NO:17 or a fragment thereof (e.g., a VP1-encoding, a VP2-encoding or a VP3-encoding fragment thereof).
369. The nucleic acid molecule of embodiment 327, wherein the nucleic acid molecule comprises a nucleotide sequence having at least 94% sequence identity to the nucleotide sequence of SEQ ID NO:17 or a fragment thereof (e.g., a VP1-encoding, a VP2-encoding or a VP3-encoding fragment thereof).
370. The nucleic acid molecule of embodiment 327, wherein the nucleic acid molecule comprises a nucleotide sequence having at least 95% sequence identity to the nucleotide sequence of SEQ ID NO:17 or a fragment thereof (e.g., a VP1-encoding, a VP2-encoding or a VP3-encoding fragment thereof).
371. The nucleic acid molecule of embodiment 327, wherein the nucleic acid molecule comprises a nucleotide sequence having at least 96% sequence identity to the nucleotide sequence of SEQ ID NO:17 or a fragment thereof (e.g., a VP1-encoding, a VP2-encoding or a VP3-encoding fragment thereof).
372. The nucleic acid molecule of embodiment 327, wherein the nucleic acid molecule comprises a nucleotide sequence having at least 97% sequence identity to the nucleotide sequence of SEQ ID NO:17 or a fragment thereof (e.g., a VP1-encoding, a VP2-encoding or a VP3-encoding fragment thereof).
373. The nucleic acid molecule of embodiment 327, wherein the nucleic acid molecule comprises a nucleotide sequence having at least 98% sequence identity to the nucleotide sequence of SEQ ID NO:17 or a fragment thereof (e.g., a VP1-encoding, a VP2-encoding or a VP3-encoding fragment thereof).
374. The nucleic acid molecule of embodiment 327, wherein the nucleic acid molecule comprises a nucleotide sequence having at least 99% sequence identity to the nucleotide sequence of SEQ ID NO:17 or a fragment thereof (e.g., a VP1-encoding, a VP2-encoding or a VP3-encoding fragment thereof).
375. The nucleic acid molecule of embodiment 327, wherein the nucleic acid molecule comprises the nucleotide sequence of SEQ ID NO:17 or a fragment thereof (e.g., a VP1-encoding, a VP2-encoding or a VP3-encoding fragment thereof).
376. The nucleic acid molecule of any one of embodiments 362 to 375, wherein the nucleic acid molecule comprises the nucleotide sequence of SEQ ID NO:17.
377. The nucleic acid molecule of any one of embodiments 362 to 375, wherein the nucleic acid molecule comprises a fragment of the nucleotide sequence of SEQ ID NO:17 that encodes a VP2 capsid polypeptide.
378. The nucleic acid molecule of any one of embodiments 362 to 375, wherein the nucleic acid molecule comprises a fragment of the nucleotide sequence of SEQ ID NO:17 that encodes a VP3 capsid polypeptide.
379. The nucleic acid molecule of embodiment 327, wherein the nucleic acid molecule comprises a nucleotide sequence having at least 70% sequence identity to the nucleotide sequence of SEQ ID NO:19 or a fragment thereof (e.g., a VP1-encoding, a VP2-encoding or a VP3-encoding fragment thereof).
380. The nucleic acid molecule of embodiment 327, wherein the nucleic acid molecule comprises a nucleotide sequence having at least 80% sequence identity to the nucleotide sequence of SEQ ID NO:19 or a fragment thereof (e.g., a VP1-encoding, a VP2-encoding or a VP3-encoding fragment thereof).
381. The nucleic acid molecule of embodiment 327, wherein the nucleic acid molecule comprises a nucleotide sequence having at least 85% sequence identity to the nucleotide sequence of SEQ ID NO:19 or a fragment thereof (e.g., a VP1-encoding, a VP2-encoding or a VP3-encoding fragment thereof).
382. The nucleic acid molecule of embodiment 327, wherein the nucleic acid molecule comprises a nucleotide sequence having at least 90% sequence identity to the nucleotide sequence of SEQ ID NO:19 or a fragment thereof (e.g., a VP1-encoding, a VP2-encoding or a VP3-encoding fragment thereof).
383. The nucleic acid molecule of embodiment 327, wherein the nucleic acid molecule comprises a nucleotide sequence having at least 91% sequence identity to the nucleotide sequence of SEQ ID NO:19 or a fragment thereof (e.g., a VP1-encoding, a VP2-encoding or a VP3-encoding fragment thereof).
384. The nucleic acid molecule of embodiment 327, wherein the nucleic acid molecule comprises a nucleotide sequence having at least 92% sequence identity to the nucleotide sequence of SEQ ID NO:19 or a fragment thereof (e.g., a VP1-encoding, a VP2-encoding or a VP3-encoding fragment thereof).
385. The nucleic acid molecule of embodiment 327, wherein the nucleic acid molecule comprises a nucleotide sequence having at least 93% sequence identity to the nucleotide sequence of SEQ ID NO:19 or a fragment thereof (e.g., a VP1-encoding, a VP2-encoding or a VP3-encoding fragment thereof).
386. The nucleic acid molecule of embodiment 327, wherein the nucleic acid molecule comprises a nucleotide sequence having at least 94% sequence identity to the nucleotide sequence of SEQ ID NO:19 or a fragment thereof (e.g., a VP1-encoding, a VP2-encoding or a VP3-encoding fragment thereof).
387. The nucleic acid molecule of embodiment 327, wherein the nucleic acid molecule comprises a nucleotide sequence having at least 95% sequence identity to the nucleotide sequence of SEQ ID NO:19 or a fragment thereof (e.g., a VP1-encoding, a VP2-encoding or a VP3-encoding fragment thereof).
388. The nucleic acid molecule of embodiment 327, wherein the nucleic acid molecule comprises a nucleotide sequence having at least 96% sequence identity to the nucleotide sequence of SEQ ID NO:19 or a fragment thereof (e.g., a VP1-encoding, a VP2-encoding or a VP3-encoding fragment thereof).
389. The nucleic acid molecule of embodiment 327, wherein the nucleic acid molecule comprises a nucleotide sequence having at least 97% sequence identity to the nucleotide sequence of SEQ ID NO:19 or a fragment thereof (e.g., a VP1-encoding, a VP2-encoding or a VP3-encoding fragment thereof).
390. The nucleic acid molecule of embodiment 327, wherein the nucleic acid molecule comprises a nucleotide sequence having at least 98% sequence identity to the nucleotide sequence of SEQ ID NO:19 or a fragment thereof (e.g., a VP1-encoding, a VP2-encoding or a VP3-encoding fragment thereof).
391. The nucleic acid molecule of embodiment 327, wherein the nucleic acid molecule comprises a nucleotide sequence having at least 99% sequence identity to the nucleotide sequence of SEQ ID NO:19 or a fragment thereof (e.g., a VP1-encoding, a VP2-encoding or a VP3-encoding fragment thereof).
392. The nucleic acid molecule of embodiment 327, wherein the nucleic acid molecule comprises the nucleotide sequence of SEQ ID NO:19 or a fragment thereof (e.g., a VP1-encoding, a VP2-encoding or a VP3-encoding fragment thereof).
393. The nucleic acid molecule of any one of embodiments 379 to 392, wherein the nucleic acid molecule comprises the nucleotide sequence of SEQ ID NO:19.
394. The nucleic acid molecule of any one of embodiments 379 to 392, wherein the nucleic acid molecule comprises a fragment of the nucleotide sequence of SEQ ID NO:19 that encodes a VP2 capsid polypeptide.
395. The nucleic acid molecule of any one of embodiments 379 to 392, wherein the nucleic acid molecule comprises a fragment of the nucleotide sequence of SEQ ID NO:19 that encodes a VP3 capsid polypeptide.
396. The nucleic acid molecule of any one of embodiments 379 to 392, wherein the nucleic acid molecule comprises a fragment of the nucleotide sequence of SEQ ID NO:19 that encodes a VP3 capsid polypeptide.
397. The nucleic acid molecule of any one of embodiments 327 to 396, wherein the nucleic acid molecule is double-stranded or single-stranded, and wherein the nucleic acid molecule is linear or circular, e.g., wherein the nucleic acid molecule is a plasmid.
398. A virus particle (e.g., adeno-associated virus (“AAV”) particle) comprising a capsid polypeptide of any one of embodiments 1 to 326 or comprising a capsid polypeptide encoded by the nucleic acid molecule of any one of embodiments 327 to 397.
399. In a virus particle (e.g., adeno-associated virus (“AAV”) particle) comprising a capsid polypeptide, the improvement comprising including in the capsid polypeptide:
   (a) a histidine at a position corresponding to R550 of the VP1 capsid polypeptide of SEQ ID NO:7;
   (b) an asparagine at a position corresponding to D551 of the VP1 capsid polypeptide of SEQ ID NO:7;
   (c) an alanine at a position corresponding to S576 of the VP1 capsid polypeptide of SEQ ID NO:7;
   (d) an isoleucine at a position corresponding to Q579 of the VP1 capsid polypeptide of SEQ ID NO:7;
   (e) a lysine at a position corresponding to H584 of the VP1 capsid polypeptide of SEQ ID NO:7;
   (f) an asparagine at a position corresponding to S586 of the VP1 capsid polypeptide of SEQ ID NO:7;
   (g) a histidine or serine at a position corresponding to A587 of the VP1 capsid polypeptide of SEQ ID NO:7;
   (h) a tyrosine, a threonine, or asparagine at a position corresponding to Q588 of the VP1 capsid polypeptide of SEQ ID NO:7;
   (i) a glutamine at a position corresponding to A589 of the VP1 capsid polypeptide of SEQ ID NO:7;
   (j) an asparagine at a position corresponding to Q592 of the VP1 capsid polypeptide of SEQ ID NO:7; or
   (k) any combination of two, three, four, five, six, seven, eight, nine, or all ten of (a), (b), (c), (d), (e), (f), (g), (h), (i), and (j), optionally wherein the combination is a combination described in any one of embodiments 3 to 67.
400. In a virus particle (e.g., adeno-associated virus (“AAV”) particle) comprising a capsid polypeptide and an encapsulated nucleic acid, the improvement comprising including in the capsid polypeptide:
   (a) a histidine at a position corresponding to R550 of the VP1 capsid polypeptide of SEQ ID NO:7;
   (b) an asparagine at a position corresponding to D551 of the VP1 capsid polypeptide of SEQ ID NO:7;
   (c) an alanine at a position corresponding to S576 of the VP1 capsid polypeptide of SEQ ID NO:7;
   (d) an isoleucine at a position corresponding to Q579 of the VP1 capsid polypeptide of SEQ ID NO:7;
   (e) a lysine at a position corresponding to H584 of the VP1 capsid polypeptide of SEQ ID NO:7;
   (f) an asparagine at a position corresponding to S586 of the VP1 capsid polypeptide of SEQ ID NO:7;
   (g) a histidine or serine at a position corresponding to A587 of the VP1 capsid polypeptide of SEQ ID NO:7;
   (h) a tyrosine, a threonine, or asparagine at a position corresponding to Q588 of the VP1 capsid polypeptide of SEQ ID NO:7;
   (i) a glutamine at a position corresponding to A589 of the VP1 capsid polypeptide of SEQ ID NO:7;
   (j) an asparagine at a position corresponding to Q592 of the VP1 capsid polypeptide of SEQ ID NO:7; or
   (k) any combination of two, three, four, five, six, seven, eight, nine, or all ten of (a), (b), (c), (d), (e), (f), (g), (h), (i), and (j), optionally wherein the combination is a combination described in any one of embodiments 3 to 67.
401. The virus particle of embodiment 398 or embodiment 399, comprising a nucleic acid comprising a payload (e.g., a heterologous transgene) and one or more regulatory elements.
402. The virus particle of embodiment 400, wherein the encapsulated nucleic acid comprises a payload (e.g., a heterologous transgene) and one or more regulatory elements.
403. The virus particle of embodiment 401 or embodiment 402, wherein the payload comprises a heterologous nucleic acid sequence as defined in any one of embodiments 433 to 1619.
404. The virus particle of any one of embodiments 398 to 403, wherein the one or more regulatory elements comprise a promoter.
405. The virus particle of embodiment 404, wherein the promoter is a constitutive promoter.
406. The virus particle of embodiment 404 or embodiment 405, wherein the promoter is a CBh promoter.
407. The virus particle of embodiment 406, wherein the CBh promoter comprises a nucleotide sequence having at least 90%, at least 95%, at least 96%, at least 97%, or at least 98%, at least 99%, or 100% sequence identity to SEQ ID NO:100.
408. The virus particle of embodiment 404 or embodiment 405, wherein the promoter is a human EF1a promoter.
409. The virus particle of embodiment 408, wherein the human EF1a promoter comprises a nucleotide sequence having at least 90%, at least 95%, at least 96%, at least 97%, or at least 98%, at least 99%, or 100% sequence identity to SEQ ID NO101.
410. The virus particle of embodiment 404, wherein the promoter is a CNS-specific promoter.
411. The virus particle of embodiment 404 or embodiment 410, wherein the promoter is a hSYN promoter.
412. The virus particle of embodiment 411, wherein the hSYN promoter comprises a nucleotide sequence having at least 90%, at least 95%, at least 96%, at least 97%, or at least 98%, at least 99%, or 100% sequence identity to SEQ ID NO102.
413. The virus particle of any one of embodiments 398 to 412, wherein said virus particle exhibits increased brain biodistribution, e.g., as measured in a mammal, e.g., in NHP, e.g., as described herein, relative to wild-type AAV9 (e.g., a virus particle comprising capsid polypeptides of SEQ ID NO:7 or encoded by SEQ ID NO:8), optionally wherein the increased brain biodistribution is measured using a method described in Section 8.1.
414. The virus particle of any one of embodiments 398 to 413, wherein said virus particle exhibits increased cardiac muscle biodistribution, e.g., as measured in a mammal, e.g., in NHP, e.g., as described herein, relative to wild-type AAV9 (e.g., a virus particle comprising capsid polypeptides of SEQ ID NO:7 or encoded by SEQ ID NO:8), optionally wherein the increased cardiac muscle biodistribution is measured using a method described in Section 8.1.
415. The virus particle of any one of embodiments 398 to 414, wherein said virus particle exhibits increased skeletal muscle biodistribution, e.g., as measured in a mammal, e.g., in NHP, e.g., as described herein, relative to wild-type AAV9 (e.g., a virus particle comprising capsid polypeptides of SEQ ID NO:7 or encoded by SEQ ID NO:8), optionally wherein the increased skeletal muscle biodistribution is measured using a method described in Section 8.1.
416. The virus particle of any one of embodiments 398 to 415, wherein said virus particle exhibits increased brain transduction, e.g., as measured in a mammal, e.g., in NHP, e.g., as described herein, relative to wild-type AAV9 (e.g., a virus particle comprising capsid polypeptides of SEQ ID NO:7 or encoded by SEQ ID NO:8), optionally wherein the increased brain transduction is measured using a method described in Section 8.1.
417. The virus particle of any one of embodiments 398 to 416, wherein said virus particle exhibits increased cardiac muscle transduction, e.g., as measured in a mammal, e.g., in NHP, e.g., as described herein, relative to wild-type AAV9 (e.g., a virus particle comprising capsid polypeptides of SEQ ID NO:7 or encoded by SEQ ID NO:8), optionally wherein the increased cardiac muscle transduction is measured using a method described in Section 8.1.
418. The virus particle of any one of embodiments 398 to 417, wherein said virus particle exhibits increased skeletal muscle transduction, e.g., as measured in a mammal, e.g., in NHP, e.g., as described herein, relative to wild-type AAV9 (e.g., a virus particle comprising capsid polypeptides of SEQ ID NO:7 or encoded by SEQ ID NO:8), optionally wherein the increased skeletal muscle transduction is measured using a method described in Section 8.1.
419. The virus particle of any one of embodiments 398 to 418, wherein said virus particle exhibits reduced liver biodistribution, e.g., as measured in a mammal, e.g., in NHP, e.g., as described herein, relative to wild-type AAV9 (e.g., a virus particle comprising capsid polypeptides of SEQ ID NO:7 or encoded by SEQ ID NO:8), optionally wherein the decreased liver biodistribution is measured using a method described in Section 8.1.
420. An adeno-associated virus particle comprising (a) a capsid polypeptide of any one of embodiments 1 to 326 or comprising a capsid polypeptide encoded by the nucleic acid molecule of any one of embodiments 327 to 397 and (b) a nucleic acid comprising a payload (e.g., a heterologous transgene) and one or more regulatory elements.
421. The virus particle of embodiment 420, wherein the virus particle comprises a capsid polypeptide comprising the amino acid sequence of SEQ ID NO:12 or the VP2 or VP3 portion thereof.
422. The virus particle of embodiment 420, wherein the virus particle comprises a capsid polypeptide comprising the amino acid sequence of SEQ ID NO:14 or the VP2 or VP3 portion thereof.
423. The virus particle of embodiment 420, wherein the virus particle comprises a capsid polypeptide comprising the amino acid sequence of SEQ ID NO:16 or the VP2 or VP3 portion thereof.
424. The virus particle of embodiment 420, wherein the virus particle comprises a capsid polypeptide comprising the amino acid sequence of SEQ ID NO:18 or the VP2 or VP3 portion thereof.
425. The virus particle of any one of embodiments 420 to 424, wherein the payload comprises a heterologous nucleic acid sequence as defined in any one of embodiments 433 to 1619.
426. The virus particle of any one of embodiments 420 to 425, wherein the one or more regulatory elements comprise a promoter.
427. The virus particle of embodiment 426, wherein the promoter is as defined in any one of embodiments 405 to 412.
428. The virus particle of any one of embodiments 398 to 427, wherein the nucleic acid comprises AAV inverted terminal repeat sequences, optionally wherein the AAV inverted repeat sequences are AAV2 inverted terminal repeat sequences.
429. The virus particle of any one of embodiments 398 to 428 (suitable) for use in gene therapy.
430. In a method of delivering a nucleic acid to a subject via an adeno-associated virus (“AAV”) particle comprising a capsid polypeptide and the nucleic acid, the improvement comprising a capsid polypeptide comprising:
   (a) a histidine at a position corresponding to R550 of the VP1 capsid polypeptide of SEQ ID NO:7;
   (b) an asparagine at a position corresponding to D551 of the VP1 capsid polypeptide of SEQ ID NO:7;
   (c) an alanine at a position corresponding to S576 of the VP1 capsid polypeptide of SEQ ID NO:7;
   (d) an isoleucine at a position corresponding to Q579 of the VP1 capsid polypeptide of SEQ ID NO:7;
   (e) a lysine at a position corresponding to H584 of the VP1 capsid polypeptide of SEQ ID NO:7;
   (f) an asparagine at a position corresponding to S586 of the VP1 capsid polypeptide of SEQ ID NO:7;
   (g) a histidine or serine at a position corresponding to A587 of the VP1 capsid polypeptide of SEQ ID NO:7;
   (h) a tyrosine, a threonine, or asparagine at a position corresponding to Q588 of the VP1 capsid polypeptide of SEQ ID NO:7;
   (i) a glutamine at a position corresponding to A589 of the VP1 capsid polypeptide of SEQ ID NO:7;
   (j) an asparagine at a position corresponding to Q592 of the VP1 capsid polypeptide of SEQ ID NO:7; or
   (k) any combination of two, three, four, five, six, seven, eight, nine, or all ten of (a), (b), (c), (d), (e), (f), (g), (h), (i), and (j), optionally wherein the combination is a combination described in any one of embodiments 3 to 67.
431. A method of treating a disease or condition in a subject, comprising administering to the subject in an amount effective to treat the disease or condition:
   (a) a virus particle:
      (i) comprising the capsid polypeptide of any one of embodiments 1 to 326 and a heterologous nucleic acid sequence encoding a therapeutic product suitable for treating the disease or condition;
      (ii) comprising a capsid polypeptide encoded by the nucleic acid molecule of any one of embodiments 327 to 397 and a heterologous nucleic acid sequence encoding a therapeutic product suitable for treating the disease or condition, or
      (iii) of any one of embodiments 398 to 429 comprising a heterologous nucleic acid sequence encoding a therapeutic product suitable for treating the disease or condition; or
   (b) a composition, e.g., a pharmaceutical composition, comprising the virus particle of (a) and, optionally, a pharmaceutically acceptable carrier.
432. In a method of treating a disease or condition in a subject with an adeno-associated virus (“AAV”) particle comprising a capsid polypeptide and a heterologous nucleic acid sequence encoding a therapeutic product suitable for treating the disease or condition, the improvement comprising a capsid polypeptide comprising
   (a) a histidine at a position corresponding to R550 of the VP1 capsid polypeptide of SEQ ID NO:7;
   (b) an asparagine at a position corresponding to D551 of the VP1 capsid polypeptide of SEQ ID NO:7;
   (c) an alanine at a position corresponding to S576 of the VP1 capsid polypeptide of SEQ ID NO:7;
   (d) an isoleucine at a position corresponding to Q579 of the VP1 capsid polypeptide of SEQ ID NO:7;
   (e) a lysine at a position corresponding to H584 of the VP1 capsid polypeptide of SEQ ID NO:7;
   (f) an asparagine at a position corresponding to S586 of the VP1 capsid polypeptide of SEQ ID NO:7;
   (g) a histidine or serine at a position corresponding to A587 of the VP1 capsid polypeptide of SEQ ID NO:7;
   (h) a tyrosine, a threonine, or asparagine at a position corresponding to Q588 of the VP1 capsid polypeptide of SEQ ID NO:7;
   (i) a glutamine at a position corresponding to A589 of the VP1 capsid polypeptide of SEQ ID NO:7;
   (j) an asparagine at a position corresponding to Q592 of the VP1 capsid polypeptide of SEQ ID NO:7; or
   (k) any combination of two, three, four, five, six, seven, eight, nine, or all ten of (a), (b), (c), (d), (e), (f), (g), (h), (i), and (j), optionally wherein the combination is a combination described in any one of embodiments 3 to 67.
433. The method of embodiment 431 or 432, wherein the disease or condition is a disease or condition of the CNS.
434. The method of any one of embodiments 431 to 433, wherein the disease or condition is selected from Absence of the Septum Pellucidum, Acid Lipase Disease, Acid Maltase Deficiency, Acquired Epileptiform Aphasia, Acute Disseminated Encephalomyelitis, Attention Deficit-Hyperactivity Disorder (ADHD), Adie's Pupil, Adie's Syndrome, Adrenoleukodystrophy, Agenesis of the Corpus Callosum, Agnosia, Aicardi Syndrome, Aicardi-Goutieres Syndrome Disorder, AIDS—Neurological Complications, Alexander Disease, Alpers' Disease, Alternating Hemiplegia, Alzheimer's Disease, Amyotrophic Lateral Sclerosis (ALS), Anencephaly, Aneurysm, Angelman Syndrome, Angiomatosis, Angleman syndrome, Anoxia, Antiphospholipid Syndrome, Aphasia, Apraxia, Arachnoid Cysts, Arachnoiditis, Arnold-Chiari Malformation, Arteriovenous Malformation, Asperger Syndrome, Ataxia, Ataxia Telangiectasia, Ataxias and Cerebellar or Spinocerebellar Degeneration, Atrial Fibrillation and Stroke, Attention Deficit-Hyperactivity Disorder, Autism Spectrum Disorder, Autonomic Dysfunction, Back Pain, Barth Syndrome, Batten Disease, Becker's Myotonia, Bechet's Disease, Bell's Palsy, Benign Essential Blepharospasm, Benign Focal Amyotrophy, Benign Intracranial Hypertension, Bernhardt-Roth Syndrome, Binswanger's Disease, Blepharospasm, Bloch-Sulzberger Syndrome, Brachial Plexus Birth Injuries, Brachial Plexus Injuries, Bradbury-Eggleston Syndrome, Brain and Spinal Tumors (including, but not limited to those that have metastasized to the brain, for example, metastatic breast cancer), Brain Aneurysm, Brain Injury, Brown-Sequard Syndrome, Bulbar palsy, Bulbospinal Muscular Atrophy, Cerebral Autosomal Dominant Arteriopathy with Sub-cortical Infarcts and Leukoencephalopathy (CADASIL), Canavan Disease, Carpal Tunnel Syndrome, Causalgia, Cavernomas, Cavernous Angioma, Cavernous Malformation, Central Cervical Cord Syndrome, Central Cord Syndrome, Central Pain Syndrome, Central Pontine Myelinolysis, Cephalic Disorders, Ceramidase Deficiency, Cerebellar Degeneration, Cerebellar Hypoplasia, Cerebral Aneurysms, Cerebral Arteriosclerosis, Cerebral Atrophy, Cerebral Beriberi, Cerebral Cavernous Malformation, Cerebral Gigantism, Cerebral Hypoxia, Cerebral Palsy, Cerebro-Oculo-Facio-Skeletal Syndrome (COFS), Charcot-Marie-Tooth Disease, Chiari Malformation, Cholesterol Ester Storage Disease, Chorea, Choreoacanthocytosis, Chronic Inflammatory Demyelinating Polyneuropathy (CIDP), Chronic Orthostatic Intolerance, Chronic Pain, Cockayne Syndrome Type II, Coffin Lowry Syndrome, Colpocephaly, Coma, Complex Regional Pain Syndrome, Concentric sclerosis (Baló's sclerosis), Congenital Facial Diplegia, Congenital Myasthenia, Congenital Myopathy, Congenital Vascular Cavernous Malformations, Corticobasal Degeneration, Cranial Arteritis, Craniosynostosis, Cree encephalitis, Creutzfeldt-Jakob Disease, Chronic progressive external ophtalmoplegia, Cumulative Trauma Disorders, Cushing's Syndrome, Cytomegalic Inclusion Body Disease, Cytomegalovirus Infection, Dancing Eyes-Dancing Feet Syndrome, Dandy-Walker Syndrome, Dawson Disease, De Morsier's Syndrome, Dejerine-Klumpke Palsy, Dementia, Dementia-Multi-Infarct, Dementia—Semantic, Dementia-Subcortical, Dementia With Lewy Bodies, Demyelination diseases, Dentate Cerebellar Ataxia, Dentatorubral Atrophy, Dermatomyositis, Developmental Dyspraxia, Devic's Syndrome, Diabetic Neuropathy, Diffuse Sclerosis, Distal hereditary motor neuronopathies, Dravet Syndrome, Dysautonomia, Dysgraphia, Dyslexia, Dysphagia, Dyspraxia, Dyssynergia Cerebellaris Myoclonica, Dyssynergia Cerebellaris Progressiva, Dystonias, Early Infantile Epileptic Encephalopathy, Empty Sella Syndrome, Encephalitis, Encephalitis Lethargica, Encephaloceles, Encephalomyelitis, Encephalopathy, Encephalopathy (familial infantile), Encephalotrigeminal Angiomatosis, Epilepsy, Epileptic Hemiplegia, Episodic ataxia, Erb's Palsy, Erb-Duchenne and Dejerine-Klumpke Palsies, Essential Tremor, Extrapontine Myelinolysis, Faber's disease, Fabry Disease, Fahr's Syndrome, Fainting, Familial Dysautonomia, Familial Hemangioma, Familial Idiopathic Basal Ganglia Calcification, Familial Periodic Paralyses, Familial Spastic Paralysis, Farber's Disease, Febrile Seizures, Fibromuscular Dysplasia, Fisher Syndrome, Floppy Infant Syndrome, Foot Drop, Fragile X disease, Friedreich's Ataxia, Frontotemporal Dementia, Gaucher Disease, Generalized Gangliosidoses (GM1, GM2), Gerstmann's Syndrome, Gerstmann-Straussler-Scheinker Disease, Giant Axonal Neuropathy, Giant Cell Arteritis, Giant Cell Inclusion Disease, Globoid Cell Leukodystrophy, Glossopharyngeal Neuralgia, Glycogen Storage Disease, Guillain-Barre Syndrome, Hallervorden-Spatz Disease, Head Injury, Headache, Hemicrania Continua, Hemifacial Spasm, Hemiplegia Alterans, Hereditary Neuropathies, Hereditary Spastic Paraplegia, Heredopathia Atactica Polyneuritiformis, Herpes Zoster, Herpes Zoster Oticus, Hirayama Syndrome, Holmes-Adie syndrome, Holoprosencephaly, HTLV-1 Associated Myelopathy, Hughes Syndrome, Huntington's Disease, Hurler syndrome, Hydranencephaly, Hydrocephalus, Hydrocephalus—Normal Pressure, Hydromyelia, Hypercortisolism, Hypersomnia, Hypertonia, Hypotonia, Hypoxia, Immune-Mediated Encephalomyelitis, Inclusion Body Myositis, Incontinentia Pigmenti, Infantile Hypotonia, Infantile Neuroaxonal Dystrophy, Infantile Phytanic Acid Storage Disease, Infantile Refsum Disease, Infantile Spasms, Inflammatory Myopathies, Iniencephaly, Intestinal Lipodystrophy, Intracranial Cysts, Intracranial Hypertension, Isaacs' Syndrome, Joubert Syndrome, Kearns-Sayre Syndrome, Kennedy's Disease, Kinsbourne syndrome, Kleine-Levin Syndrome, Klippel-Feil Syndrome, Klippel-Trenaunay Syndrome (KTS), Kluver-Bucy Syndrome, Korsakoff's Amnesic Syndrome, Krabbe Disease, Kugelberg-Welander Disease, Kuru, Lambert-Eaton Myasthenic Syndrome, Landau-Kleffner Syndrome, Lateral Femoral Cutaneous Nerve Entrapment, Lateral Medullary Syndrome, Learning Disabilities, Leigh's Disease, Lennox-Gastaut Syndrome, Lesch-Nyhan Syndrome, Leukodystrophy, Levine-Critchley Syndrome, Lewy Body Dementia, Lichtheim's disease, Lipid Storage Diseases, Lipoid Proteinosis, Lissencephaly, Locked-In Syndrome, Lou Gehrig's Disease, Lupus—Neurological Sequelae, Lyme Disease—Neurological Complications, Lysosomal storage disorders, Machado-Joseph Disease, Macrencephaly, Megalencephaly, Melkersson-Rosenthal Syndrome, Meningitis, Meningitis and Encephalitis, Menkes Disease, Meralgia Paresthetica, Metachromatic Leukodystrophy, Microcephaly, Migraine, Miller Fisher Syndrome, Mini Stroke, Mitochondrial Myopathy, Mitochondrial DNA depletion syndromes, Moebius Syndrome, Monomelic Amyotrophy, Morvan Syndrome, Motor Neuron Diseases, Moyamoya Disease, Mucolipidoses, Mucopolysaccharidoses, Multi-Infarct Dementia, Multifocal Motor Neuropathy, Multiple Sclerosis, Multiple System Atrophy, Multiple System Atrophy with Orthostatic Hypotension, Muscular Dystrophy, Myasthenia—Congenital, Myasthenia Gravis, Myelinoclastic Diffuse Sclerosis, Myelitis, Myoclonic Encephalopathy of Infants, Myoclonus, Myoclonus epilepsy, Myopathy, Myopathy-Congenital, Myopathy-Thyrotoxic, Myotonia, Myotonia Congenita, Narcolepsy, NARP (neuropathy, ataxia and retinitis pigmentosa), Neuroacanthocytosis, Neurodegeneration with Brain Iron Accumulation, Neurodegenerative disease, Neurofibromatosis, Neuroleptic Malignant Syndrome, Neurological Complications of AIDS, Neurological Complications of Lyme Disease, Neurological Consequences of Cytomegalovirus Infection, Neurological Manifestations of Pompe Disease, Neurological Sequelae Of Lupus, Neuromyelitis Optica, Neuromyotonia, Neuronal Ceroid Lipofuscinosis, Neuronal Migration Disorders, Neuropathic pain, Neuropathy—Hereditary, Neuropathy, Neurosarcoidosis, Neurosyphilis, Neurotoxicity, Nevus Cavernosus, Niemann-Pick Disease, O'Sullivan-McLeod Syndrome, Occipital Neuralgia, Ohtahara Syndrome, Olivopontocerebellar Atrophy, Opsoclonus Myoclonus, Orthostatic Hypotension, Overuse Syndrome, Pain—Chronic, Pantothenate Kinase-Associated Neurodegeneration, Paraneoplastic Syndromes, Paresthesia, Parkinson's Disease, Paroxysmal Choreoathetosis, Paroxysmal Hemicrania, Parry-Romberg, Pelizaeus-Merzbacher Disease, Pena Shokeir II Syndrome, Perineural Cysts, Peroneal muscular atrophy, Periodic Paralyses, Peripheral Neuropathy, Periventricular Leukomalacia, Persistent Vegetative State, Pervasive Developmental Disorders, Phelan McDermid syndrome, Phytanic Acid Storage Disease, Pick's Disease, Pinched Nerve, Piriformis Syndrome, Pituitary Tumors, Polymyositis, Pompe Disease, Porencephaly, Post-Polio Syndrome, Postherpetic Neuralgia, Postinfectious Encephalomyelitis, Postural Hypotension, Postural Orthostatic Tachycardia Syndrome, Postural Tachycardia Syndrome, Primary Dentatum Atrophy, Primary Lateral Sclerosis, Primary Progressive Aphasia, Prion Diseases, Progressive bulbar palsy, Progressive Hemifacial Atrophy, Progressive Locomotor Ataxia, Progressive Multifocal Leukoencephalopathy, Progressive Muscular Atrophy, Progressive Sclerosing Poliodystrophy, Progressive Supranuclear Palsy, Prosopagnosia, Pseudobulbar palsy, Pseudo—Torch syndrome, Pseudotoxoplasmosis syndrome, Pseudotumor Cerebri, Psychogenic Movement, Ramsay Hunt Syndrome I, Ramsay Hunt Syndrome II, Rasmussen's Encephalitis, Reflex Sympathetic Dystrophy Syndrome, Refsum Disease, Refsum Disease Infantile, Repetitive Motion Disorders, Repetitive Stress Injuries, Restless Legs Syndrome, Retrovirus-Associated Myelopathy, Rett Syndrome, Reye's Syndrome, Rheumatic Encephalitis, Riley-Day Syndrome, Sacral Nerve Root Cysts, Saint Vitus Dance, Salivary Gland Disease, Sandhoff Disease, Schilder's Disease, Schizencephaly, Seitelberger Disease, Seizure Disorder, Semantic Dementia, Septo-Optic Dysplasia, Severe Myoclonic Epilepsy of Infancy (SMEI), Shaken Baby Syndrome, Shingles, Shy-Drager Syndrome, Sjögren's Syndrome, Sleep Apnea, Sleeping Sickness, Sotos Syndrome, Spasticity, Spina Bifida, Spinal Cord Infarction, Spinal Cord Injury, Spinal Cord Tumors, Spinal Muscular Atrophy, Spinocerebellar Ataxia, Spinocerebellar Atrophy, Spinocerebellar Degeneration, Sporadic ataxia, Steele-Richardson-Olszewski Syndrome, Stiff-Person Syndrome, Striatonigral Degeneration, Stroke, Sturge-Weber Syndrome, Subacute Sclerosing Panencephalitis, Subcortical Arteriosclerotic Encephalopathy, Short-lasting, Unilateral, Neuralgiform (SUNCT) Headache, Swallowing Disorders, Sydenham Chorea, Syncope, Syphilitic Spinal Sclerosis, Syringohydromyelia, Syringomyelia, Systemic Lupus Erythematosus, Tabes Dorsalis, Tardive Dyskinesia, Tarlov Cysts, Tay-Sachs Disease, Temporal Arteritis, Tethered Spinal Cord Syndrome, Thomsen's Myotonia, Thoracic Outlet Syndrome, Thyrotoxic Myopathy, Tic Douloureux, Todd's Paralysis, Tourette Syndrome, Transient Ischemic Attack, Transmissible Spongiform Encephalopathies, Transverse Myelitis, Traumatic Brain Injury, Tremor, Trigeminal Neuralgia, Tropical Spastic Paraparesis, Troyer Syndrome, Tuberous Sclerosis, Vascular Erectile Tumor, Vasculitis Syndromes of the Central and Peripheral Nervous Systems, Vitamin B12 deficiency, Von Economo's Disease, Von Hippel-Lindau Disease (VHL), Von Recklinghausen's Disease, Wallenberg's Syndrome, Werdnig-Hoffman Disease, Wernicke-Korsakoff Syndrome, West Syndrome, Whiplash, Whipple's Disease, Williams Syndrome, Wilson Disease, Wolman's Disease, and X-Linked Spinal or Bulbar Muscular Atrophy.
435. The method of any one of embodiments 431 to 434, wherein the disease or condition is Acid Lipase Disease.
436. The method of any one of embodiments 431 to 434, wherein the disease or condition is Acid Maltase Deficiency.
437. The method of any one of embodiments 431 to 434, wherein the disease or condition is Acquired Epileptiform Aphasia.
438. The method of any one of embodiments 431 to 434, wherein the disease or condition is Acute Disseminated Encephalomyelitis.
439. The method of any one of embodiments 431 to 434, wherein the disease or condition is Attention Deficit-Hyperactivity Disorder (ADHD).
440. The method of any one of embodiments 431 to 434, wherein the disease or condition is Adie's Pupil.
441. The method of any one of embodiments 431 to 434, wherein the disease or condition is Adie's Syndrome.
442. The method of any one of embodiments 431 to 434, wherein the disease or condition is Adrenoleukodystrophy.
443. The method of any one of embodiments 431 to 434, wherein the disease or condition is Agenesis of the Corpus Callosum.
444. The method of any one of embodiments 431 to 434, wherein the disease or condition is Agnosia.
445. The method of any one of embodiments 431 to 434, wherein the disease or condition is Aicardi Syndrome.
446. The method of any one of embodiments 431 to 434, wherein the disease or condition is Aicardi-Goutieres Syndrome Disorder.
447. The method of any one of embodiments 431 to 434, wherein the disease or condition is AIDS—Neurological Complications.
448. The method of any one of embodiments 431 to 434, wherein the disease or condition is Alexander Disease.
449. The method of any one of embodiments 431 to 434, wherein the disease or condition is Alpers' Disease.
450. The method of any one of embodiments 431 to 434, wherein the disease or condition is Alternating Hemiplegia.
451. The method of any one of embodiments 431 to 434, wherein the disease or condition is Alzheimer's Disease.
452. The method of any one of embodiments 431 to 434, wherein the disease or condition is Amyotrophic Lateral Sclerosis (ALS).
453. The method of any one of embodiments 431 to 434, wherein the disease or condition is Anencephaly.
454. The method of any one of embodiments 431 to 434, wherein the disease or condition is Aneurysm.
455. The method of any one of embodiments 431 to 434, wherein the disease or condition is Angelman Syndrome.
456. The method of any one of embodiments 431 to 434, wherein the disease or condition is Angiomatosis.
457. The method of any one of embodiments 431 to 434, wherein the disease or condition is Angleman syndrome.
458. The method of any one of embodiments 431 to 434, wherein the disease or condition is Anoxia.
459. The method of any one of embodiments 431 to 434, wherein the disease or condition is Antiphospholipid Syndrome.
460. The method of any one of embodiments 431 to 434, wherein the disease or condition is Aphasia.
461. The method of any one of embodiments 431 to 434, wherein the disease or condition is Apraxia.
462. The method of any one of embodiments 431 to 434, wherein the disease or condition is Arachnoid Cysts.
463. The method of any one of embodiments 431 to 434, wherein the disease or condition is Arachnoiditis.
464. The method of any one of embodiments 431 to 434, wherein the disease or condition is Arnold-Chiari Malformation.
465. The method of any one of embodiments 431 to 434, wherein the disease or condition is Arteriovenous Malformation.
466. The method of any one of embodiments 431 to 434, wherein the disease or condition is Asperger Syndrome.
467. The method of any one of embodiments 431 to 434, wherein the disease or condition is Ataxia.
468. The method of any one of embodiments 431 to 434, wherein the disease or condition is Ataxia Telangiectasia.
469. The method of any one of embodiments 431 to 434, wherein the disease or condition is Ataxia and Cerebellar or Spinocerebellar Degeneration.
470. The method of any one of embodiments 431 to 434, wherein the disease or condition is Atrial Fibrillation and Stroke.
471. The method of any one of embodiments 431 to 434, wherein the disease or condition is Attention Deficit-Hyperactivity Disorder.
472. The method of any one of embodiments 431 to 434, wherein the disease or condition is Autism Spectrum Disorder.
473. The method of any one of embodiments 431 to 434, wherein the disease or condition is Autonomic Dysfunction.
474. The method of any one of embodiments 431 to 434, wherein the disease or condition is Back Pain.
475. The method of any one of embodiments 431 to 434, wherein the disease or condition is Barth Syndrome.
476. The method of any one of embodiments 431 to 434, wherein the disease or condition is Batten Disease.
477. The method of any one of embodiments 431 to 434, wherein the disease or condition is Becker's Myotonia.
478. The method of any one of embodiments 431 to 434, wherein the disease or condition is Bechet's Disease.
479. The method of any one of embodiments 431 to 434, wherein the disease or condition is Bell's Palsy.
480. The method of any one of embodiments 431 to 434, wherein the disease or condition is Benign Essential Blepharospasm.
481. The method of any one of embodiments 431 to 434, wherein the disease or condition is Benign Focal Amyotrophy.
482. The method of any one of embodiments 431 to 434, wherein the disease or condition is Benign Intracranial Hypertension.
483. The method of any one of embodiments 431 to 434, wherein the disease or condition is Bernhardt-Roth Syndrome.
484. The method of any one of embodiments 431 to 434, wherein the disease or condition is Binswanger's Disease.
485. The method of any one of embodiments 431 to 434, wherein the disease or condition is Blepharospasm.
486. The method of any one of embodiments 431 to 434, wherein the disease or condition is Bloch-Sulzberger Syndrome.
487. The method of any one of embodiments 431 to 434, wherein the disease or condition is Brachial Plexus Birth Injury.
488. The method of any one of embodiments 431 to 434, wherein the disease or condition is Brachial Plexus Injury.
489. The method of any one of embodiments 431 to 434, wherein the disease or condition is Bradbury-Eggleston Syndrome.
490. The method of any one of embodiments 431 to 434, wherein the disease or condition is a Brain or Spinal Tumor (including, but not limited to a tumor that has metastasized to the brain, for example, metastatic breast cancer).
491. The method of any one of embodiments 431 to 434, wherein the disease or condition is Brain Aneurysm.
492. The method of any one of embodiments 431 to 434, wherein the disease or condition is Brain Injury.
493. The method of any one of embodiments 431 to 434, wherein the disease or condition is Brown-Sequard Syndrome.
494. The method of any one of embodiments 431 to 434, wherein the disease or condition is Bulbar palsy.
495. The method of any one of embodiments 431 to 434, wherein the disease or condition is Bulbospinal Muscular Atrophy.
496. The method of any one of embodiments 431 to 434, wherein the disease or condition is Cerebral Autosomal Dominant Arteriopathy with Sub-cortical Infarcts and Leukoencephalopathy (CADASIL).
497. The method of any one of embodiments 431 to 434, wherein the disease or condition is Canavan Disease.
498. The method of any one of embodiments 431 to 434, wherein the disease or condition is Carpal Tunnel Syndrome.
499. The method of any one of embodiments 431 to 434, wherein the disease or condition is Causalgia.
500. The method of any one of embodiments 431 to 434, wherein the disease or condition is Cavernomas.
501. The method of any one of embodiments 431 to 434, wherein the disease or condition is Cavernous Angioma.
502. The method of any one of embodiments 431 to 434, wherein the disease or condition is Cavernous Malformation.
503. The method of any one of embodiments 431 to 434, wherein the disease or condition is Central Cervical Cord Syndrome.
504. The method of any one of embodiments 431 to 434, wherein the disease or condition is Central Cord Syndrome.
505. The method of any one of embodiments 431 to 434, wherein the disease or condition is Central Pain Syndrome.
506. The method of any one of embodiments 431 to 434, wherein the disease or condition is Central Pontine Myelinolysis.
507. The method of any one of embodiments 431 to 434, wherein the disease or condition is a Cephalic Disorder.
508. The method of any one of embodiments 431 to 434, wherein the disease or condition is Ceramidase Deficiency.
509. The method of any one of embodiments 431 to 434, wherein the disease or condition is Cerebellar Degeneration.
510. The method of any one of embodiments 431 to 434, wherein the disease or condition is Cerebellar Hypoplasia.
511. The method of any one of embodiments 431 to 434, wherein the disease or condition is a Cerebral Aneurysm.
512. The method of any one of embodiments 431 to 434, wherein the disease or condition is Cerebral Arteriosclerosis.
513. The method of any one of embodiments 431 to 434, wherein the disease or condition is Cerebral Atrophy.
514. The method of any one of embodiments 431 to 434, wherein the disease or condition is Cerebral Beriberi.
515. The method of any one of embodiments 431 to 434, wherein the disease or condition is Cerebral Cavernous Malformation.
516. The method of any one of embodiments 431 to 434, wherein the disease or condition is Cerebral Gigantism.
517. The method of any one of embodiments 431 to 434, wherein the disease or condition is Cerebral Hypoxia.
518. The method of any one of embodiments 431 to 434, wherein the disease or condition is Cerebral Palsy.
519. The method of any one of embodiments 431 to 434, wherein the disease or condition is Cerebro-Oculo-Facio-Skeletal Syndrome (COFS).
520. The method of any one of embodiments 431 to 434, wherein the disease or condition is Charcot-Marie-Tooth Disease.
521. The method of any one of embodiments 431 to 434, wherein the disease or condition is Chiari Malformation.
522. The method of any one of embodiments 431 to 434, wherein the disease or condition is Cholesterol Ester Storage Disease.
523. The method of any one of embodiments 431 to 434, wherein the disease or condition is Chorea.
524. The method of any one of embodiments 431 to 434, wherein the disease or condition is Choreoacanthocytosis.
525. The method of any one of embodiments 431 to 434, wherein the disease or condition is Chronic Inflammatory Demyelinating Polyneuropathy (CIDP).
526. The method of any one of embodiments 431 to 434, wherein the disease or condition is Chronic Orthostatic Intolerance.
527. The method of any one of embodiments 431 to 434, wherein the disease or condition is Chronic Pain.
528. The method of any one of embodiments 431 to 434, wherein the disease or condition is Cockayne Syndrome Type II.
529. The method of any one of embodiments 431 to 434, wherein the disease or condition is Coffin Lowry Syndrome.
530. The method of any one of embodiments 431 to 434, wherein the disease or condition is Colpocephaly.
531. The method of any one of embodiments 431 to 434, wherein the disease or condition is Coma.
532. The method of any one of embodiments 431 to 434, wherein the disease or condition is Complex Regional Pain Syndrome.
533. The method of any one of embodiments 431 to 434, wherein the disease or condition is Concentric sclerosis (Baló's sclerosis).
534. The method of any one of embodiments 431 to 434, wherein the disease or condition is Congenital Facial Diplegia.
535. The method of any one of embodiments 431 to 434, wherein the disease or condition is Congenital Myasthenia.
536. The method of any one of embodiments 431 to 434, wherein the disease or condition is Congenital Myopathy.
537. The method of any one of embodiments 431 to 434, wherein the disease or condition is Congenital Vascular Cavernous Malformations.
538. The method of any one of embodiments 431 to 434, wherein the disease or condition is Corticobasal Degeneration.
539. The method of any one of embodiments 431 to 434, wherein the disease or condition is Cranial Arteritis.
540. The method of any one of embodiments 431 to 434, wherein the disease or condition is Craniosynostosis.
541. The method of any one of embodiments 431 to 434, wherein the disease or condition is Cree encephalitis.
542. The method of any one of embodiments 431 to 434, wherein the disease or condition is Creutzfeldt-Jakob Disease.
543. The method of any one of embodiments 431 to 434, wherein the disease or condition is Chronic progressive external ophtalmoplegia.
544. The method of any one of embodiments 431 to 434, wherein the disease or condition is a Cumulative Trauma Disorder.
545. The method of any one of embodiments 431 to 434, wherein the disease or condition is Cushing's Syndrome.
546. The method of any one of embodiments 431 to 434, wherein the disease or condition is Cytomegalic Inclusion Body Disease.
547. The method of any one of embodiments 431 to 434, wherein the disease or condition is Cytomegalovirus Infection.
548. The method of any one of embodiments 431 to 434, wherein the disease or condition is Dancing Eyes-Dancing Feet Syndrome.
549. The method of any one of embodiments 431 to 434, wherein the disease or condition is Dandy-Walker Syndrome.
550. The method of any one of embodiments 431 to 434, wherein the disease or condition is Dawson Disease.
551. The method of any one of embodiments 431 to 434, wherein the disease or condition is De Morsier's Syndrome.
552. The method of any one of embodiments 431 to 434, wherein the disease or condition is Dejerine-Klumpke Palsy.
553. The method of any one of embodiments 431 to 434, wherein the disease or condition is Dementia.
554. The method of any one of embodiments 431 to 434, wherein the disease or condition is Dementia-Multi-Infarct.
555. The method of any one of embodiments 431 to 434, wherein the disease or condition is Dementia-Semantic.
556. The method of any one of embodiments 431 to 434, wherein the disease or condition is Dementia-Subcortical.
557. The method of any one of embodiments 431 to 434, wherein the disease or condition is Dementia With Lewy Bodies.
558. The method of any one of embodiments 431 to 434, wherein the disease or condition is a Demyelination disease.
559. The method of any one of embodiments 431 to 434, wherein the disease or condition is Dentate Cerebellar Ataxia.
560. The method of any one of embodiments 431 to 434, wherein the disease or condition is Dentatorubral Atrophy.
561. The method of any one of embodiments 431 to 434, wherein the disease or condition is Dermatomyositis.
562. The method of any one of embodiments 431 to 434, wherein the disease or condition is Developmental Dyspraxia.
563. The method of any one of embodiments 431 to 434, wherein the disease or condition is Devic's Syndrome.
564. The method of any one of embodiments 431 to 434, wherein the disease or condition is Diabetic Neuropathy.
565. The method of any one of embodiments 431 to 434, wherein the disease or condition is Diffuse Sclerosis.
566. The method of any one of embodiments 431 to 434, wherein the disease or condition is a Distal hereditary motor neuronopathy.
567. The method of any one of embodiments 431 to 434, wherein the disease or condition is Dravet Syndrome.
568. The method of any one of embodiments 431 to 434, wherein the disease or condition is Dysautonomia.
569. The method of any one of embodiments 431 to 434, wherein the disease or condition is Dysgraphia.
570. The method of any one of embodiments 431 to 434, wherein the disease or condition is Dyslexia.
571. The method of any one of embodiments 431 to 434, wherein the disease or condition is Dysphagia.
572. The method of any one of embodiments 431 to 434, wherein the disease or condition is Dyspraxia.
573. The method of any one of embodiments 431 to 434, wherein the disease or condition is Dyssynergia Cerebellaris Myoclonica.
574. The method of any one of embodiments 431 to 434, wherein the disease or condition is Dyssynergia Cerebellaris Progressiva.
575. The method of any one of embodiments 431 to 434, wherein the disease or condition is Dystonias.
576. The method of any one of embodiments 431 to 434, wherein the disease or condition is Early Infantile Epileptic Encephalopathy.
577. The method of any one of embodiments 431 to 434, wherein the disease or condition is Empty Sella Syndrome.
578. The method of any one of embodiments 431 to 434, wherein the disease or condition is Encephalitis.
579. The method of any one of embodiments 431 to 434, wherein the disease or condition is Encephalitis Lethargica.
580. The method of any one of embodiments 431 to 434, wherein the disease or condition is Encephaloceles.
581. The method of any one of embodiments 431 to 434, wherein the disease or condition is Encephalomyelitis.
582. The method of any one of embodiments 431 to 434, wherein the disease or condition is Encephalopathy.
583. The method of any one of embodiments 431 to 434, wherein the disease or condition is Encephalopathy (familial infantile).
584. The method of any one of embodiments 431 to 434, wherein the disease or condition is Encephalotrigeminal Angiomatosis.
585. The method of any one of embodiments 431 to 434, wherein the disease or condition is Epilepsy.
586. The method of any one of embodiments 431 to 434, wherein the disease or condition is Epileptic Hemiplegia.
587. The method of any one of embodiments 431 to 434, wherein the disease or condition is Episodic ataxia.
588. The method of any one of embodiments 431 to 434, wherein the disease or condition is Erb's Palsy.
589. The method of any one of embodiments 431 to 434, wherein the disease or condition is Erb-Duchenne Palsy
590. The method of any one of embodiments 431 to 434, wherein the disease or condition is Dejerine-Klumpke Palsy.
591. The method of any one of embodiments 431 to 434, wherein the disease or condition is Essential Tremor.
592. The method of any one of embodiments 431 to 434, wherein the disease or condition is Extrapontine Myelinolysis.
593. The method of any one of embodiments 431 to 434, wherein the disease or condition is Faber's disease.
594. The method of any one of embodiments 431 to 434, wherein the disease or condition is Fabry Disease.
595. The method of any one of embodiments 431 to 434, wherein the disease or condition is Fahr's Syndrome.
596. The method of any one of embodiments 431 to 434, wherein the disease or condition is Fainting.
597. The method of any one of embodiments 431 to 434, wherein the disease or condition is Familial Dysautonomia.
598. The method of any one of embodiments 431 to 434, wherein the disease or condition is Familial Hemangioma.
599. The method of any one of embodiments 431 to 434, wherein the disease or condition is Familial Idiopathic Basal Ganglia Calcification.
600. The method of any one of embodiments 431 to 434, wherein the disease or condition is Familial Periodic Paralyses.
601. The method of any one of embodiments 431 to 434, wherein the disease or condition is Familial Spastic Paralysis.
602. The method of any one of embodiments 431 to 434, wherein the disease or condition is Farber's Disease.
603. The method of any one of embodiments 431 to 434, wherein the disease or condition is Febrile Seizures.
604. The method of any one of embodiments 431 to 434, wherein the disease or condition is Fibromuscular Dysplasia.
605. The method of any one of embodiments 431 to 434, wherein the disease or condition is Fisher Syndrome.
606. The method of any one of embodiments 431 to 434, wherein the disease or condition is Floppy Infant Syndrome.
607. The method of any one of embodiments 431 to 434, wherein the disease or condition is Foot Drop.
608. The method of any one of embodiments 431 to 434, wherein the disease or condition is Fragile X disease.
609. The method of any one of embodiments 431 to 434, wherein the disease or condition is Friedreich's Ataxia.
610. The method of any one of embodiments 431 to 434, wherein the disease or condition is Frontotemporal Dementia.
611. The method of any one of embodiments 431 to 434, wherein the disease or condition is Gaucher Disease.
612. The method of any one of embodiments 431 to 434, wherein the disease or condition is Generalized Gangliosidoses (GM1, GM2).
613. The method of any one of embodiments 431 to 434, wherein the disease or condition is Gerstmann's Syndrome.
614. The method of any one of embodiments 431 to 434, wherein the disease or condition is Gerstmann-Straussler-Scheinker Disease.
615. The method of any one of embodiments 431 to 434, wherein the disease or condition is Giant Axonal Neuropathy.
616. The method of any one of embodiments 431 to 434, wherein the disease or condition is Giant Cell Arteritis.
617. The method of any one of embodiments 431 to 434, wherein the disease or condition is Giant Cell Inclusion Disease.
618. The method of any one of embodiments 431 to 434, wherein the disease or condition is Globoid Cell Leukodystrophy.
619. The method of any one of embodiments 431 to 434, wherein the disease or condition is Glossopharyngeal Neuralgia.
620. The method of any one of embodiments 431 to 434, wherein the disease or condition is Glycogen Storage Disease.
621. The method of any one of embodiments 431 to 434, wherein the disease or condition is Guillain-Barre Syndrome.
622. The method of any one of embodiments 431 to 434, wherein the disease or condition is Hallervorden-Spatz Disease.
623. The method of any one of embodiments 431 to 434, wherein the disease or condition is a Head Injury.
624. The method of any one of embodiments 431 to 434, wherein the disease or condition is Headache.
625. The method of any one of embodiments 431 to 434, wherein the disease or condition is Hemicrania Continua.
626. The method of any one of embodiments 431 to 434, wherein the disease or condition is Hemifacial Spasm.
627. The method of any one of embodiments 431 to 434, wherein the disease or condition is Hemiplegia Alterans.
628. The method of any one of embodiments 431 to 434, wherein the disease or condition is a Hereditary Neuropathy.
629. The method of any one of embodiments 431 to 434, wherein the disease or condition is Hereditary Spastic Paraplegia.
630. The method of any one of embodiments 431 to 434, wherein the disease or condition is Heredopathia Atactica Polyneuritiformis.
631. The method of any one of embodiments 431 to 434, wherein the disease or condition is Herpes Zoster.
632. The method of any one of embodiments 431 to 434, wherein the disease or condition is Herpes Zoster Oticus.
633. The method of any one of embodiments 431 to 434, wherein the disease or condition is Hirayama Syndrome.
634. The method of any one of embodiments 431 to 434, wherein the disease or condition is Holmes-Adie syndrome.
635. The method of any one of embodiments 431 to 434, wherein the disease or condition is Holoprosencephaly.
636. The method of any one of embodiments 431 to 434, wherein the disease or condition is HTLV-1 Associated Myelopathy.
637. The method of any one of embodiments 431 to 434, wherein the disease or condition is Hughes Syndrome.
638. The method of any one of embodiments 431 to 434, wherein the disease or condition is Huntington's Disease.
639. The method of any one of embodiments 431 to 434, wherein the disease or condition is Hurler syndrome.
640. The method of any one of embodiments 431 to 434, wherein the disease or condition is Hydranencephaly.
641. The method of any one of embodiments 431 to 434, wherein the disease or condition is Hydrocephalus.
642. The method of any one of embodiments 431 to 434, wherein the disease or condition is Hydrocephalus-Normal Pressure.
643. The method of any one of embodiments 431 to 434, wherein the disease or condition is Hydromyelia.
644. The method of any one of embodiments 431 to 434, wherein the disease or condition is Hypercortisolism.
645. The method of any one of embodiments 431 to 434, wherein the disease or condition is Hypersomnia.
646. The method of any one of embodiments 431 to 434, wherein the disease or condition is Hypertonia.
647. The method of any one of embodiments 431 to 434, wherein the disease or condition is Hypotonia.
648. The method of any one of embodiments 431 to 434, wherein the disease or condition is Hypoxia.
649. The method of any one of embodiments 431 to 434, wherein the disease or condition is Immune-Mediated Encephalomyelitis.
650. The method of any one of embodiments 431 to 434, wherein the disease or condition is Inclusion Body Myositis.
651. The method of any one of embodiments 431 to 434, wherein the disease or condition is Incontinentia Pigmenti.
652. The method of any one of embodiments 431 to 434, wherein the disease or condition is Infantile Hypotonia.
653. The method of any one of embodiments 431 to 434, wherein the disease or condition is Infantile Neuroaxonal Dystrophy.
654. The method of any one of embodiments 431 to 434, wherein the disease or condition is Infantile Phytanic Acid Storage Disease.
655. The method of any one of embodiments 431 to 434, wherein the disease or condition is Infantile Refsum Disease.
656. The method of any one of embodiments 431 to 434, wherein the disease or condition is Infantile Spasms.
657. The method of any one of embodiments 431 to 434, wherein the disease or condition is an Inflammatory Myopathy.
658. The method of any one of embodiments 431 to 434, wherein the disease or condition is Iniencephaly.
659. The method of any one of embodiments 431 to 434, wherein the disease or condition is Intestinal Lipodystrophy.
660. The method of any one of embodiments 431 to 434, wherein the disease or condition is Intracranial Cysts.
661. The method of any one of embodiments 431 to 434, wherein the disease or condition is Intracranial Hypertension.
662. The method of any one of embodiments 431 to 434, wherein the disease or condition is Isaacs' Syndrome.
663. The method of any one of embodiments 431 to 434, wherein the disease or condition is Joubert Syndrome.
664. The method of any one of embodiments 431 to 434, wherein the disease or condition is Kearns-Sayre Syndrome.
665. The method of any one of embodiments 431 to 434, wherein the disease or condition is Kennedy's Disease.
666. The method of any one of embodiments 431 to 434, wherein the disease or condition is Kinsbourne syndrome.
667. The method of any one of embodiments 431 to 434, wherein the disease or condition is Kleine-Levin Syndrome.
668. The method of any one of embodiments 431 to 434, wherein the disease or condition is Klippel-Feil Syndrome.
669. The method of any one of embodiments 431 to 434, wherein the disease or condition is Klippel-Trenaunay Syndrome (KTS).
670. The method of any one of embodiments 431 to 434, wherein the disease or condition is Klüver-Bucy Syndrome.
671. The method of any one of embodiments 431 to 434, wherein the disease or condition is Korsakoffs Amnesic Syndrome.
672. The method of any one of embodiments 431 to 434, wherein the disease or condition is Krabbe Disease.
673. The method of any one of embodiments 431 to 434, wherein the disease or condition is Kugelberg-Welander Disease.
674. The method of any one of embodiments 431 to 434, wherein the disease or condition is Kuru.
675. The method of any one of embodiments 431 to 434, wherein the disease or condition is Lambert-Eaton Myasthenic Syndrome.
676. The method of any one of embodiments 431 to 434, wherein the disease or condition is Landau-Kleffner Syndrome.
677. The method of any one of embodiments 431 to 434, wherein the disease or condition is Lateral Femoral Cutaneous Nerve Entrapment.
678. The method of any one of embodiments 431 to 434, wherein the disease or condition is Lateral Medullary Syndrome.
679. The method of any one of embodiments 431 to 434, wherein the disease or condition is a Learning Disability.
680. The method of any one of embodiments 431 to 434, wherein the disease or condition is Leigh's Disease.
681. The method of any one of embodiments 431 to 434, wherein the disease or condition is Lennox-Gastaut Syndrome.
682. The method of any one of embodiments 431 to 434, wherein the disease or condition is Lesch-Nyhan Syndrome.
683. The method of any one of embodiments 431 to 434, wherein the disease or condition is Leukodystrophy.
684. The method of any one of embodiments 431 to 434, wherein the disease or condition is Levine-Critchley Syndrome.
685. The method of any one of embodiments 431 to 434, wherein the disease or condition is Lewy Body Dementia.
686. The method of any one of embodiments 431 to 434, wherein the disease or condition is Lichtheim's disease.
687. The method of any one of embodiments 431 to 434, wherein the disease or condition is a Lipid Storage Disease.
688. The method of any one of embodiments 431 to 434, wherein the disease or condition is Lipoid Proteinosis.
689. The method of any one of embodiments 431 to 434, wherein the disease or condition is Lissencephaly.
690. The method of any one of embodiments 431 to 434, wherein the disease or condition is Locked-In Syndrome.
691. The method of any one of embodiments 431 to 434, wherein the disease or condition is Lou Gehrig's Disease.
692. The method of any one of embodiments 431 to 434, wherein the disease or condition is Lupus-Neurological Sequelae.
693. The method of any one of embodiments 431 to 434, wherein the disease or condition is Lyme Disease-Neurological Complications.
694. The method of any one of embodiments 431 to 434, wherein the disease or condition is Lysosomal storage disorders.
695. The method of any one of embodiments 431 to 434, wherein the disease or condition is Machado-Joseph Disease.
696. The method of any one of embodiments 431 to 434, wherein the disease or condition is Macrencephaly.
697. The method of any one of embodiments 431 to 434, wherein the disease or condition is Megalencephaly.
698. The method of any one of embodiments 431 to 434, wherein the disease or condition is Melkersson-Rosenthal Syndrome.
699. The method of any one of embodiments 431 to 434, wherein the disease or condition is Meningitis.
700. The method of any one of embodiments 431 to 434, wherein the disease or condition is Meningitis and Encephalitis.
701. The method of any one of embodiments 431 to 434, wherein the disease or condition is Menkes Disease.
702. The method of any one of embodiments 431 to 434, wherein the disease or condition is Meralgia Paresthetica.
703. The method of any one of embodiments 431 to 434, wherein the disease or condition is Metachromatic Leukodystrophy.
704. The method of any one of embodiments 431 to 434, wherein the disease or condition is Microcephaly.
705. The method of any one of embodiments 431 to 434, wherein the disease or condition is Migraine.
706. The method of any one of embodiments 431 to 434, wherein the disease or condition is Miller Fisher Syndrome.
707. The method of any one of embodiments 431 to 434, wherein the disease or condition is Mini Stroke.
708. The method of any one of embodiments 431 to 434, wherein the disease or condition is Mitochondrial Myopathy.
709. The method of any one of embodiments 431 to 434, wherein the disease or condition is a Mitochondrial DNA depletion syndrome.
710. The method of any one of embodiments 431 to 434, wherein the disease or condition is Moebius Syndrome.
711. The method of any one of embodiments 431 to 434, wherein the disease or condition is Monomelic Amyotrophy.
712. The method of any one of embodiments 431 to 434, wherein the disease or condition is Morvan Syndrome.
713. The method of any one of embodiments 431 to 434, wherein the disease or condition is a Motor Neuron Disease.
714. The method of any one of embodiments 431 to 434, wherein the disease or condition is Moyamoya Disease.
715. The method of any one of embodiments 431 to 434, wherein the disease or condition is Mucolipidoses.
716. The method of any one of embodiments 431 to 434, wherein the disease or condition is Mucopolysaccharidoses.
717. The method of any one of embodiments 431 to 434, wherein the disease or condition is Multi-Infarct Dementia.
718. The method of any one of embodiments 431 to 434, wherein the disease or condition is Multifocal Motor Neuropathy.
719. The method of any one of embodiments 431 to 434, wherein the disease or condition is Multiple Sclerosis.
720. The method of any one of embodiments 431 to 434, wherein the disease or condition is Multiple System Atrophy.
721. The method of any one of embodiments 431 to 434, wherein the disease or condition is Multiple System Atrophy with Orthostatic Hypotension.
722. The method of any one of embodiments 431 to 434, wherein the disease or condition is Muscular Dystrophy.
723. The method of any one of embodiments 431 to 434, wherein the disease or condition is Myasthenia-Congenital.
724. The method of any one of embodiments 431 to 434, wherein the disease or condition is Myasthenia Gravis.
725. The method of any one of embodiments 431 to 434, wherein the disease or condition is Myelinoclastic Diffuse Sclerosis.
726. The method of any one of embodiments 431 to 434, wherein the disease or condition is Myelitis.
727. The method of any one of embodiments 431 to 434, wherein the disease or condition is Myoclonic Encephalopathy of Infants.
728. The method of any one of embodiments 431 to 434, wherein the disease or condition is Myoclonus.
729. The method of any one of embodiments 431 to 434, wherein the disease or condition is Myoclonus epilepsy.
730. The method of any one of embodiments 431 to 434, wherein the disease or condition is Myopathy.
731. The method of any one of embodiments 431 to 434, wherein the disease or condition is Myopathy-Congenital.
732. The method of any one of embodiments 431 to 434, wherein the disease or condition is Myopathy-Thyrotoxic.
733. The method of any one of embodiments 431 to 434, wherein the disease or condition is Myotonia.
734. The method of any one of embodiments 431 to 434, wherein the disease or condition is Myotonia Congenita.
735. The method of any one of embodiments 431 to 434, wherein the disease or condition is Narcolepsy.
736. The method of any one of embodiments 431 to 434, wherein the disease or condition is NARP (neuropathy, ataxia and retinitis pigmentosa).
737. The method of any one of embodiments 431 to 434, wherein the disease or condition is Neuroacanthocytosis.
738. The method of any one of embodiments 431 to 434, wherein the disease or condition is Neurodegeneration with Brain Iron Accumulation.
739. The method of any one of embodiments 431 to 434, wherein the disease or condition is a Neurodegenerative disease.
740. The method of any one of embodiments 431 to 434, wherein the disease or condition is Neurofibromatosis.
741. The method of any one of embodiments 431 to 434, wherein the disease or condition is Neuroleptic Malignant Syndrome.
742. The method of any one of embodiments 431 to 434, wherein the disease or condition is Neurological Complications of AIDS.
743. The method of any one of embodiments 431 to 434, wherein the disease or condition is Neurological Complications of Lyme Disease.
744. The method of any one of embodiments 431 to 434, wherein the disease or condition is Neurological Consequences of Cytomegalovirus Infection.
745. The method of any one of embodiments 431 to 434, wherein the disease or condition is Neurological Manifestations of Pompe Disease.
746. The method of any one of embodiments 431 to 434, wherein the disease or condition is Neurological Sequelae Of Lupus.
747. The method of any one of embodiments 431 to 434, wherein the disease or condition is Neuromyelitis Optica.
748. The method of any one of embodiments 431 to 434, wherein the disease or condition is Neuromyotonia.
749. The method of any one of embodiments 431 to 434, wherein the disease or condition is Neuronal Ceroid Lipofuscinosis.
750. The method of any one of embodiments 431 to 434, wherein the disease or condition is Neuronal Migration Disorders.
751. The method of any one of embodiments 431 to 434, wherein the disease or condition is Neuropathic pain.
752. The method of any one of embodiments 431 to 434, wherein the disease or condition is Neuropathy-Hereditary.
753. The method of any one of embodiments 431 to 434, wherein the disease or condition is Neuropathy.
754. The method of any one of embodiments 431 to 434, wherein the disease or condition is Neurosarcoidosis.
755. The method of any one of embodiments 431 to 434, wherein the disease or condition is Neurosyphilis.
756. The method of any one of embodiments 431 to 434, wherein the disease or condition is Neurotoxicity.
757. The method of any one of embodiments 431 to 434, wherein the disease or condition is Nevus Cavernosus.
758. The method of any one of embodiments 431 to 434, wherein the disease or condition is Niemann-Pick Disease.
759. The method of any one of embodiments 431 to 434, wherein the disease or condition is O'Sullivan-McLeod Syndrome.
760. The method of any one of embodiments 431 to 434, wherein the disease or condition is Occipital Neuralgia.
761. The method of any one of embodiments 431 to 434, wherein the disease or condition is Ohtahara Syndrome.
762. The method of any one of embodiments 431 to 434, wherein the disease or condition is Olivopontocerebellar Atrophy.
763. The method of any one of embodiments 431 to 434, wherein the disease or condition is Opsoclonus Myoclonus.
764. The method of any one of embodiments 431 to 434, wherein the disease or condition is Orthostatic Hypotension.
765. The method of any one of embodiments 431 to 434, wherein the disease or condition is Overuse Syndrome.
766. The method of any one of embodiments 431 to 434, wherein the disease or condition is Pain-Chronic.
767. The method of any one of embodiments 431 to 434, wherein the disease or condition is Pantothenate Kinase-Associated Neurodegeneration.
768. The method of any one of embodiments 431 to 434, wherein the disease or condition is Paraneoplastic Syndromes.
769. The method of any one of embodiments 431 to 434, wherein the disease or condition is Paresthesia.
770. The method of any one of embodiments 431 to 434, wherein the disease or condition is Parkinson's Disease.
771. The method of any one of embodiments 431 to 434, wherein the disease or condition is Paroxysmal Choreoathetosis.
772. The method of any one of embodiments 431 to 434, wherein the disease or condition is Paroxysmal Hemicrania.
773. The method of any one of embodiments 431 to 434, wherein the disease or condition is Parry-Romberg.
774. The method of any one of embodiments 431 to 434, wherein the disease or condition is Pelizaeus-Merzbacher Disease.
775. The method of any one of embodiments 431 to 434, wherein the disease or condition is Pena Shokeir II Syndrome.
776. The method of any one of embodiments 431 to 434, wherein the disease or condition is Perineural Cysts.
777. The method of any one of embodiments 431 to 434, wherein the disease or condition is Peroneal muscular atrophy.
778. The method of any one of embodiments 431 to 434, wherein the disease or condition is Periodic Paralyses.
779. The method of any one of embodiments 431 to 434, wherein the disease or condition is Peripheral Neuropathy.
780. The method of any one of embodiments 431 to 434, wherein the disease or condition is Periventricular Leukomalacia.
781. The method of any one of embodiments 431 to 434, wherein the disease or condition is Persistent Vegetative State.
782. The method of any one of embodiments 431 to 434, wherein the disease or condition is a Pervasive Developmental Disorder.
783. The method of any one of embodiments 431 to 434, wherein the disease or condition is Phelan McDermid syndrome.
784. The method of any one of embodiments 431 to 434, wherein the disease or condition is Phytanic Acid Storage Disease.
785. The method of any one of embodiments 431 to 434, wherein the disease or condition is Pick's Disease.
786. The method of any one of embodiments 431 to 434, wherein the disease or condition is Pinched Nerve.
787. The method of any one of embodiments 431 to 434, wherein the disease or condition is Piriformis Syndrome.
788. The method of any one of embodiments 431 to 434, wherein the disease or condition is a Pituitary Tumor.
789. The method of any one of embodiments 431 to 434, wherein the disease or condition is Polymyositis.
790. The method of any one of embodiments 431 to 434, wherein the disease or condition is Pompe Disease.
791. The method of any one of embodiments 431 to 434, wherein the disease or condition is Porencephaly.
792. The method of any one of embodiments 431 to 434, wherein the disease or condition is Post-Polio Syndrome.
793. The method of any one of embodiments 431 to 434, wherein the disease or condition is Postherpetic Neuralgia.
794. The method of any one of embodiments 431 to 434, wherein the disease or condition is Postinfectious Encephalomyelitis.
795. The method of any one of embodiments 431 to 434, wherein the disease or condition is Postural Hypotension.
796. The method of any one of embodiments 431 to 434, wherein the disease or condition is Postural Orthostatic Tachycardia Syndrome.
797. The method of any one of embodiments 431 to 434, wherein the disease or condition is Postural Tachycardia Syndrome.
798. The method of any one of embodiments 431 to 434, wherein the disease or condition is Primary Dentatum Atrophy.
799. The method of any one of embodiments 431 to 434, wherein the disease or condition is Primary Lateral Sclerosis.
800. The method of any one of embodiments 431 to 434, wherein the disease or condition is Primary Progressive Aphasia.
801. The method of any one of embodiments 431 to 434, wherein the disease or condition is a Prion Disease.
802. The method of any one of embodiments 431 to 434, wherein the disease or condition is Progressive bulbar palsy.
803. The method of any one of embodiments 431 to 434, wherein the disease or condition is Progressive Hemifacial Atrophy.
804. The method of any one of embodiments 431 to 434, wherein the disease or condition is Progressive Locomotor Ataxia.
805. The method of any one of embodiments 431 to 434, wherein the disease or condition is Progressive Multifocal Leukoencephalopathy.
806. The method of any one of embodiments 431 to 434, wherein the disease or condition is Progressive Muscular Atrophy.
807. The method of any one of embodiments 431 to 434, wherein the disease or condition is Progressive Sclerosing Poliodystrophy.
808. The method of any one of embodiments 431 to 434, wherein the disease or condition is Progressive Supranuclear Palsy.
809. The method of any one of embodiments 431 to 434, wherein the disease or condition is Prosopagnosia.
810. The method of any one of embodiments 431 to 434, wherein the disease or condition is Pseudobulbar palsy.
811. The method of any one of embodiments 431 to 434, wherein the disease or condition is Pseudo-Torch syndrome.
812. The method of any one of embodiments 431 to 434, wherein the disease or condition is Pseudotoxoplasmosis syndrome.
813. The method of any one of embodiments 431 to 434, wherein the disease or condition is Pseudotumor Cerebri.
814. The method of any one of embodiments 431 to 434, wherein the disease or condition is Psychogenic Movement.
815. The method of any one of embodiments 431 to 434, wherein the disease or condition is Ramsay Hunt Syndrome I.
816. The method of any one of embodiments 431 to 434, wherein the disease or condition is Ramsay Hunt Syndrome II.
817. The method of any one of embodiments 431 to 434, wherein the disease or condition is Rasmussen's Encephalitis.
818. The method of any one of embodiments 431 to 434, wherein the disease or condition is Reflex Sympathetic Dystrophy Syndrome.
819. The method of any one of embodiments 431 to 434, wherein the disease or condition is Refsum Disease.
820. The method of any one of embodiments 431 to 434, wherein the disease or condition is Refsum Disease-Infantile.
821. The method of any one of embodiments 431 to 434, wherein the disease or condition is a Repetitive Motion Disorder.
822. The method of any one of embodiments 431 to 434, wherein the disease or condition is a Repetitive Stress Injury.
823. The method of any one of embodiments 431 to 434, wherein the disease or condition is Restless Legs Syndrome.
824. The method of any one of embodiments 431 to 434, wherein the disease or condition is Retrovirus-Associated Myelopathy.
825. The method of any one of embodiments 431 to 434, wherein the disease or condition is Rett Syndrome.
826. The method of any one of embodiments 431 to 434, wherein the disease or condition is Reye's Syndrome.
827. The method of any one of embodiments 431 to 434, wherein the disease or condition is Rheumatic Encephalitis.
828. The method of any one of embodiments 431 to 434, wherein the disease or condition is Riley-Day Syndrome.
829. The method of any one of embodiments 431 to 434, wherein the disease or condition is Sacral Nerve Root Cysts.
830. The method of any one of embodiments 431 to 434, wherein the disease or condition is Saint Vitus Dance.
831. The method of any one of embodiments 431 to 434, wherein the disease or condition is Salivary Gland Disease.
832. The method of any one of embodiments 431 to 434, wherein the disease or condition is Sandhoff Disease.
833. The method of any one of embodiments 431 to 434, wherein the disease or condition is Schilder's Disease.
834. The method of any one of embodiments 431 to 434, wherein the disease or condition is Schizencephaly.
835. The method of any one of embodiments 431 to 434, wherein the disease or condition is Seitelberger Disease.
836. The method of any one of embodiments 431 to 434, wherein the disease or condition is Seizure Disorder.
837. The method of any one of embodiments 431 to 434, wherein the disease or condition is Semantic Dementia.
838. The method of any one of embodiments 431 to 434, wherein the disease or condition is Septo-Optic Dysplasia.
839. The method of any one of embodiments 431 to 434, wherein the disease or condition is Severe Myoclonic Epilepsy of Infancy (SMEI).
840. The method of any one of embodiments 431 to 434, wherein the disease or condition is Shaken Baby Syndrome.
841. The method of any one of embodiments 431 to 434, wherein the disease or condition is Shingles.
842. The method of any one of embodiments 431 to 434, wherein the disease or condition is Shy-Drager Syndrome.
843. The method of any one of embodiments 431 to 434, wherein the disease or condition is Sjögren's Syndrome.
844. The method of any one of embodiments 431 to 434, wherein the disease or condition is Sleep Apnea.
845. The method of any one of embodiments 431 to 434, wherein the disease or condition is Sleeping Sickness.
846. The method of any one of embodiments 431 to 434, wherein the disease or condition is Sotos Syndrome.
847. The method of any one of embodiments 431 to 434, wherein the disease or condition is Spasticity.
848. The method of any one of embodiments 431 to 434, wherein the disease or condition is Spina Bifida.
849. The method of any one of embodiments 431 to 434, wherein the disease or condition is Spinal Cord Infarction.
850. The method of any one of embodiments 431 to 434, wherein the disease or condition is Spinal Cord Injury.
851. The method of any one of embodiments 431 to 434, wherein the disease or condition is a Spinal Cord Tumor.
852. The method of any one of embodiments 431 to 434, wherein the disease or condition is Spinal Muscular Atrophy.
853. The method of any one of embodiments 431 to 434, wherein the disease or condition is Spinocerebellar Ataxia.
854. The method of any one of embodiments 431 to 434, wherein the disease or condition is Spinocerebellar Atrophy.
855. The method of any one of embodiments 431 to 434, wherein the disease or condition is Spinocerebellar Degeneration.
856. The method of any one of embodiments 431 to 434, wherein the disease or condition is Sporadic ataxia.
857. The method of any one of embodiments 431 to 434, wherein the disease or condition is Steele-Richardson-Olszewski Syndrome.
858. The method of any one of embodiments 431 to 434, wherein the disease or condition is Stiff-Person Syndrome.
859. The method of any one of embodiments 431 to 434, wherein the disease or condition is Striatonigral Degeneration.
860. The method of any one of embodiments 431 to 434, wherein the disease or condition is Stroke.
861. The method of any one of embodiments 431 to 434, wherein the disease or condition is Sturge-Weber Syndrome.
862. The method of any one of embodiments 431 to 434, wherein the disease or condition is Subacute Sclerosing Panencephalitis.
863. The method of any one of embodiments 431 to 434, wherein the disease or condition is Subcortical Arteriosclerotic Encephalopathy.
864. The method of any one of embodiments 431 to 434, wherein the disease or condition is Short-lasting, Unilateral, Neuralgiform (SUNCT) Headache.
865. The method of any one of embodiments 431 to 434, wherein the disease or condition is a Swallowing Disorder.
866. The method of any one of embodiments 431 to 434, wherein the disease or condition is Sydenham Chorea.
867. The method of any one of embodiments 431 to 434, wherein the disease or condition is Syncope.
868. The method of any one of embodiments 431 to 434, wherein the disease or condition is Syphilitic Spinal Sclerosis.
869. The method of any one of embodiments 431 to 434, wherein the disease or condition is Syringohydromyelia.
870. The method of any one of embodiments 431 to 434, wherein the disease or condition is Syringomyelia.
871. The method of any one of embodiments 431 to 434, wherein the disease or condition is Systemic Lupus Erythematosus.
872. The method of any one of embodiments 431 to 434, wherein the disease or condition is Tabes Dorsalis.
873. The method of any one of embodiments 431 to 434, wherein the disease or condition is Tardive Dyskinesia.
874. The method of any one of embodiments 431 to 434, wherein the disease or condition is Tarlov Cysts.
875. The method of any one of embodiments 431 to 434, wherein the disease or condition is Tay-Sachs Disease.
876. The method of any one of embodiments 431 to 434, wherein the disease or condition is Temporal Arteritis.
877. The method of any one of embodiments 431 to 434, wherein the disease or condition is Tethered Spinal Cord Syndrome.
878. The method of any one of embodiments 431 to 434, wherein the disease or condition is Thomsen's Myotonia.
879. The method of any one of embodiments 431 to 434, wherein the disease or condition is Thoracic Outlet Syndrome.
880. The method of any one of embodiments 431 to 434, wherein the disease or condition is Thyrotoxic Myopathy.
881. The method of any one of embodiments 431 to 434, wherein the disease or condition is Tic Douloureux.
882. The method of any one of embodiments 431 to 434, wherein the disease or condition is Todd's Paralysis.
883. The method of any one of embodiments 431 to 434, wherein the disease or condition is Tourette Syndrome.
884. The method of any one of embodiments 431 to 434, wherein the disease or condition is Transient Ischemic Attack.
885. The method of any one of embodiments 431 to 434, wherein the disease or condition is Transmissible Spongiform Encephalopathies.
886. The method of any one of embodiments 431 to 434, wherein the disease or condition is Transverse Myelitis.
887. The method of any one of embodiments 431 to 434, wherein the disease or condition is Traumatic Brain Injury.
888. The method of any one of embodiments 431 to 434, wherein the disease or condition is Tremor.
889. The method of any one of embodiments 431 to 434, wherein the disease or condition is Trigeminal Neuralgia.
890. The method of any one of embodiments 431 to 434, wherein the disease or condition is Tropical Spastic Paraparesis.
891. The method of any one of embodiments 431 to 434, wherein the disease or condition is Troyer Syndrome.
892. The method of any one of embodiments 431 to 434, wherein the disease or condition is Tuberous Sclerosis.
893. The method of any one of embodiments 431 to 434, wherein the disease or condition is Vascular Erectile Tumor.
894. The method of any one of embodiments 431 to 434, wherein the disease or condition is a Vasculitis Syndrome of the Central or Peripheral Nervous System.
895. The method of any one of embodiments 431 to 434, wherein the disease or condition is Vitamin B12 deficiency.
896. The method of any one of embodiments 431 to 434, wherein the disease or condition is Von Economo's Disease.
897. The method of any one of embodiments 431 to 434, wherein the disease or condition is Von Hippel-Lindau Disease (VHL).
898. The method of any one of embodiments 431 to 434, wherein the disease or condition is Von Recklinghausen's Disease.
899. The method of any one of embodiments 431 to 434, wherein the disease or condition is Wallenberg's Syndrome.
900. The method of any one of embodiments 431 to 434, wherein the disease or condition is Werdnig-Hoffman Disease.
901. The method of any one of embodiments 431 to 434, wherein the disease or condition is Wernicke-Korsakoff Syndrome.
902. The method of any one of embodiments 431 to 434, wherein the disease or condition is West Syndrome.
903. The method of any one of embodiments 431 to 434, wherein the disease or condition is Whiplash.
904. The method of any one of embodiments 431 to 434, wherein the disease or condition is Whipple's Disease.
905. The method of any one of embodiments 431 to 434, wherein the disease or condition is Williams Syndrome.
906. The method of any one of embodiments 431 to 434, wherein the disease or condition is Wilson Disease.
907. The method of any one of embodiments 431 to 434, wherein the disease or condition is Wolman's Disease.
908. The method of any one of embodiments 431 to 434, wherein the disease or condition is X-Linked Spinal or Bulbar Muscular Atrophy.
909. The method of any one of embodiments 431 to 434, wherein the disease or condition is Achromatopsia (color blindness) and/or wherein the heterologous nucleic acid sequence encodes Cyclic nucleotide-gated cation channel alpha-3 (CNGA3) (e.g., a polypeptide represented by UniProt Accession number Q16281 or a polypeptide comprising an amino acid sequence having at least 95% sequence identity thereto).
910. The method of any one of embodiments 431 to 434, wherein the disease or condition is Achromatopsia (color blindness) and/or wherein the heterologous nucleic acid sequence encodes Cyclic nucleotide-gated cation channel beta-3 (CNGB3) (e.g., a polypeptide represented by UniProt Accession number Q9NQW8 or a polypeptide comprising an amino acid sequence having at least 95% sequence identity thereto).
911. The method of any one of embodiments 431 to 434, wherein the disease or condition is Achromatopsia (color blindness) and/or wherein the heterologous nucleic acid sequence encodes Guanine nucleotide-binding protein G(t) subunit alpha-2 (GNAT2) (e.g., a polypeptide represented by UniProt Accession number P19087 or a polypeptide comprising an amino acid sequence having at least 95% sequence identity thereto).
912. The method of any one of embodiments 431 to 434, wherein the disease or condition is Achromatopsia (color blindness) and/or wherein the heterologous nucleic acid sequence encodes Cone cGMP-specific 3′,5′-cyclic phosphodiesterase subunit alpha (PDE6C) (e.g., a polypeptide represented by UniProt Accession number P51160 or a polypeptide comprising an amino acid sequence having at least 95% sequence identity thereto).
913. The method of any one of embodiments 431 to 434, wherein the disease or condition is Acute Intermittent Porphyria and/or wherein the heterologous nucleic acid sequence encodes Porphobilinogen deaminase (PBGD), HMBS (e.g., a polypeptide represented by UniProt Accession number P08397 or a polypeptide comprising an amino acid sequence having at least 95% sequence identity thereto).
914. The method of any one of embodiments 431 to 434, wherein the disease or condition is Adie syndrome (Adie's pupil) and/or wherein the heterologous nucleic acid sequence encodes MPZ (e.g., a polypeptide represented by UniProt Accession number P25189 or a polypeptide comprising an amino acid sequence having at least 95% sequence identity thereto).
915. The method of any one of embodiments 431 to 434, wherein the disease or condition is Age-Related Macular Degeneration and/or wherein the heterologous nucleic acid sequence encodes Vascular endothelial growth factor receptor 1 (FLT1) (e.g., a polypeptide represented by UniProt Accession number P17948 or a polypeptide comprising an amino acid sequence having at least 95% sequence identity thereto).
916. The method of any one of embodiments 431 to 434, wherein the disease or condition is Age-Related Macular Degeneration and/or wherein the heterologous nucleic acid sequence encodes Vascular endothelial growth factor A (VEGFA) (e.g., a polypeptide represented by UniProt Accession number P15692 or a polypeptide comprising an amino acid sequence having at least 95% sequence identity thereto).
917. The method of any one of embodiments 431 to 434, wherein the disease or condition is Agenesis of the Corpus Callosum (ACCPN) and/or wherein the heterologous nucleic acid sequence encodes SLC12A6 (e.g., a polypeptide represented by UniProt Accession number Q9UHW9 or a polypeptide comprising an amino acid sequence having at least 95% sequence identity thereto).
918. The method of any one of embodiments 431 to 434, wherein the disease or condition is Aicardi-Goutieres syndrome and/or wherein the heterologous nucleic acid sequence encodes TREX1 (e.g., a polypeptide represented by UniProt Accession number Q9NSU2 or a polypeptide comprising an amino acid sequence having at least 95% sequence identity thereto).
919. The method of any one of embodiments 431 to 434, wherein the disease or condition is Alexander disease and/or wherein the heterologous nucleic acid sequence encodes GFAP (e.g., a polypeptide represented by UniProt Accession number P14136 or a polypeptide comprising an amino acid sequence having at least 95% sequence identity thereto).
920. The method of any one of embodiments 431 to 434, wherein the disease or condition is Alpers syndrome and/or wherein the heterologous nucleic acid sequence encodes POLG (e.g., a polypeptide represented by UniProt Accession number P54098 or a polypeptide comprising an amino acid sequence having at least 95% sequence identity thereto).
921. The method of any one of embodiments 431 to 434, wherein the disease or condition is Alternating hemiplegia and/or wherein the heterologous nucleic acid sequence encodes ATP1A2 (e.g., a polypeptide represented by UniProt Accession number P50993 or a polypeptide comprising an amino acid sequence having at least 95% sequence identity thereto).
922. The method of any one of embodiments 431 to 434, wherein the disease or condition is Alternating hemiplegia and/or wherein the heterologous nucleic acid sequence encodes ATP1A3 (e.g., a polypeptide represented by UniProt Accession number P13637 or a polypeptide comprising an amino acid sequence having at least 95% sequence identity thereto).
923. The method of any one of embodiments 431 to 434, wherein the disease or condition is Alzheimer's disease and/or wherein the heterologous nucleic acid sequence encodes NGF (e.g., a polypeptide represented by UniProt Accession number P01138 or a polypeptide comprising an amino acid sequence having at least 95% sequence identity thereto).
924. The method of any one of embodiments 431 to 434, wherein the disease or condition is Alzheimer's disease and/or wherein the heterologous nucleic acid sequence encodes ApoE (e.g., a polypeptide represented by UniProt Accession number P02649 or a polypeptide comprising an amino acid sequence having at least 95% sequence identity thereto).
925. The method of any one of embodiments 431 to 434, wherein the disease or condition is Alzheimer's disease and/or wherein the heterologous nucleic acid sequence encodes Presenilin (PSEN1) (e.g., a polypeptide represented by UniProt Accession number A0A024R6A3 or a polypeptide comprising an amino acid sequence having at least 95% sequence identity thereto).
926. The method of any one of embodiments 431 to 434, wherein the disease or condition is Alzheimer's disease and/or wherein the heterologous nucleic acid sequence encodes Presenilin-2 (PSEN2) (e.g., a polypeptide represented by UniProt Accession number P49810 or a polypeptide comprising an amino acid sequence having at least 95% sequence identity thereto).
927. The method of any one of embodiments 431 to 434, wherein the disease or condition is Alzheimer's disease and/or wherein the heterologous nucleic acid sequence encodes Amyloid-beta precursor protein (APP) (e.g., a polypeptide represented by UniProt Accession number P05067 or a polypeptide comprising an amino acid sequence having at least 95% sequence identity thereto).
928. The method of any one of embodiments 431 to 434, wherein the disease or condition is Alzheimer's disease and/or wherein the heterologous nucleic acid sequence encodes ADAM10 (e.g., a polypeptide represented by UniProt Accession number O14672 or a polypeptide comprising an amino acid sequence having at least 95% sequence identity thereto).
929. The method of any one of embodiments 431 to 434, wherein the disease or condition is Alzheimer's disease and/or wherein the heterologous nucleic acid sequence encodes MAPT, Tau (e.g., a polypeptide represented by UniProt Accession number P10636 or a polypeptide comprising an amino acid sequence having at least 95% sequence identity thereto).
930. The method of any one of embodiments 431 to 434, wherein the disease or condition is Amyotrophic lateral sclerosis (ALS) (Lou Gehrig's disease) and/or wherein the heterologous nucleic acid sequence encodes Superoxide dismutase-1 (SOD1) (e.g., a polypeptide represented by UniProt Accession number P00441 or a polypeptide comprising an amino acid sequence having at least 95% sequence identity thereto).
931. The method of any one of embodiments 431 to 434, wherein the disease or condition is Amyotrophy, hereditary neuralgic and/or wherein the heterologous nucleic acid sequence encodes 45544 (e.g., a polypeptide represented by UniProt Accession number Q9UHD8 or a polypeptide comprising an amino acid sequence having at least 95% sequence identity thereto).
932. The method of any one of embodiments 431 to 434, wherein the disease or condition is Angleman syndrome and/or wherein the heterologous nucleic acid sequence encodes Ubiquitin-protein ligase E3A (UBE3A) (e.g., a polypeptide represented by UniProt Accession number Q05086 or a polypeptide comprising an amino acid sequence having at least 95% sequence identity thereto).
933. The method of any one of embodiments 431 to 434, wherein the disease or condition is Aromatic L-amino acid decarboxylase deficiency (AADCD) and/or wherein the heterologous nucleic acid sequence encodes DDC (e.g., a polypeptide represented by UniProt Accession number P20711 or a polypeptide comprising an amino acid sequence having at least 95% sequence identity thereto).
934. The method of any one of embodiments 431 to 434, wherein the disease or condition is Ataxia and/or wherein the heterologous nucleic acid sequence encodes APTX (e.g., a polypeptide represented by UniProt Accession number Q7Z2E3 or a polypeptide comprising an amino acid sequence having at least 95% sequence identity thereto).
935. The method of any one of embodiments 431 to 434, wherein the disease or condition is Ataxia and/or wherein the heterologous nucleic acid sequence encodes KCNA1 (e.g., a polypeptide represented by UniProt Accession number Q09470 or a polypeptide comprising an amino acid sequence having at least 95% sequence identity thereto).
936. The method of any one of embodiments 431 to 434, wherein the disease or condition is Ataxia and/or wherein the heterologous nucleic acid sequence encodes CACNA1A (e.g., a polypeptide represented by UniProt Accession number O00555 or a polypeptide comprising an amino acid sequence having at least 95% sequence identity thereto).
937. The method of any one of embodiments 431 to 434, wherein the disease or condition is Ataxia Telangiectasia (Louis-Bar syndrome) and/or wherein the heterologous nucleic acid sequence encodes Serine-protein kinase ATM (ATM) (e.g., a polypeptide represented by UniProt Accession number Q13315 or a polypeptide comprising an amino acid sequence having at least 95% sequence identity thereto).
938. The method of any one of embodiments 431 to 434, wherein the disease or condition is Attention deficit hyperactivity disorder (ADHD) and/or wherein the heterologous nucleic acid sequence encodes DRD4 (e.g., a polypeptide represented by UniProt Accession number P21917 or a polypeptide comprising an amino acid sequence having at least 95% sequence identity thereto).
939. The method of any one of embodiments 431 to 434, wherein the disease or condition is Attention deficit hyperactivity disorder (ADHD) and/or wherein the heterologous nucleic acid sequence encodes CDH2 (e.g., a polypeptide represented by UniProt Accession number P19022 or a polypeptide comprising an amino acid sequence having at least 95% sequence identity thereto).
940. The method of any one of embodiments 431 to 434, wherein the disease or condition is Becker muscular dystrophy and/or wherein the heterologous nucleic acid sequence encodes Follistatin (FST) (e.g., a polypeptide represented by UniProt Accession number P19883 or a polypeptide comprising an amino acid sequence having at least 95% sequence identity thereto).
941. The method of any one of embodiments 431 to 434, wherein the disease or condition is Becker muscular dystrophy and/or wherein the heterologous nucleic acid sequence encodes DMD (e.g., a polypeptide represented by UniProt Accession number P11532 or a polypeptide comprising an amino acid sequence having at least 95% sequence identity thereto).
942. The method of any one of embodiments 431 to 434, wherein the disease or condition is Benign essential blepharospasm and/or wherein the heterologous nucleic acid sequence encodes DRD5 (e.g., a polypeptide represented by UniProt Accession number P21918 or a polypeptide comprising an amino acid sequence having at least 95% sequence identity thereto).
943. The method of any one of embodiments 431 to 434, wherein the disease or condition is Bradbury-Eggleston syndrome (pure autonomic failure) and/or wherein the heterologous nucleic acid sequence encodes COQ2 (e.g., a polypeptide represented by UniProt Accession number Q96H96 or a polypeptide comprising an amino acid sequence having at least 95% sequence identity thereto).
944. The method of any one of embodiments 431 to 434, wherein the disease or condition is Bulbar palsy (BVVLS1) and/or wherein the heterologous nucleic acid sequence encodes SLC52A3 (e.g., a polypeptide represented by UniProt Accession number Q9NQ40 or a polypeptide comprising an amino acid sequence having at least 95% sequence identity thereto).
945. The method of any one of embodiments 431 to 434, wherein the disease or condition is Canavan disease (aminoacylase 2 deficiency) and/or wherein the heterologous nucleic acid sequence encodes ASPA (e.g., a polypeptide represented by UniProt Accession number P45381 or a polypeptide comprising an amino acid sequence having at least 95% sequence identity thereto).
946. The method of any one of embodiments 431 to 434, wherein the disease or condition is Carpal tunnel syndrome and/or wherein the heterologous nucleic acid sequence encodes TTR (e.g., a polypeptide represented by UniProt Accession number P02766 or a polypeptide comprising an amino acid sequence having at least 95% sequence identity thereto).
947. The method of any one of embodiments 431 to 434, wherein the disease or condition is Cavernoma (Cavernous angioma, Cavernous malformations) and/or wherein the heterologous nucleic acid sequence encodes KRIT1 (e.g., a polypeptide represented by UniProt Accession number O00522 or a polypeptide comprising an amino acid sequence having at least 95% sequence identity thereto).
948. The method of any one of embodiments 431 to 434, wherein the disease or condition is Cerebellar Hypoplasia (CHEGDD) and/or wherein the heterologous nucleic acid sequence encodes OXR1 (e.g., a polypeptide represented by UniProt Accession number Q8N573 or a polypeptide comprising an amino acid sequence having at least 95% sequence identity thereto).
949. The method of any one of embodiments 431 to 434, wherein the disease or condition is Cerebellar ataxia (CAMRQ2) and/or wherein the heterologous nucleic acid sequence encodes WDR81 (e.g., a polypeptide represented by UniProt Accession number Q562E7 or a polypeptide comprising an amino acid sequence having at least 95% sequence identity thereto).
950. The method of any one of embodiments 431 to 434, wherein the disease or condition is Cerebral Arteriopathy with SCI and Leukoencephalopathy (CADASIL) and/or wherein the heterologous nucleic acid sequence encodes NOTCH3 (e.g., a polypeptide represented by UniProt Accession number Q9UM47 or a polypeptide comprising an amino acid sequence having at least 95% sequence identity thereto).
951. The method of any one of embodiments 431 to 434, wherein the disease or condition is Cerebral gigantism (Sotos syndrome 1) and/or wherein the heterologous nucleic acid sequence encodes NSD1 (e.g., a polypeptide represented by UniProt Accession number Q96L73 or a polypeptide comprising an amino acid sequence having at least 95% sequence identity thereto).
952. The method of any one of embodiments 431 to 434, wherein the disease or condition is Cerebro-oculo-facio-skeletal syndrome (COFS) and/or wherein the heterologous nucleic acid sequence encodes ERCC6 (e.g., a polypeptide represented by UniProt Accession number Q03468 or a polypeptide comprising an amino acid sequence having at least 95% sequence identity thereto).
953. The method of any one of embodiments 431 to 434, wherein the disease or condition is Ceroid lipofuscinosis (Batten disease) and/or wherein the heterologous nucleic acid sequence encodes CLN1 (PPT1) (e.g., a polypeptide represented by UniProt Accession number P50897 or a polypeptide comprising an amino acid sequence having at least 95% sequence identity thereto).
954. The method of any one of embodiments 431 to 434, wherein the disease or condition is Ceroid lipofuscinosis (Batten disease) and/or wherein the heterologous nucleic acid sequence encodes CLN2 (TPP1) (e.g., a polypeptide represented by UniProt Accession number O14773 or a polypeptide comprising an amino acid sequence having at least 95% sequence identity thereto).
955. The method of any one of embodiments 431 to 434, wherein the disease or condition is Ceroid lipofuscinosis (Batten disease) and/or wherein the heterologous nucleic acid sequence encodes CLN3 (battenin) (e.g., a polypeptide represented by UniProt Accession number Q13286 or a polypeptide comprising an amino acid sequence having at least 95% sequence identity thereto).
956. The method of any one of embodiments 431 to 434, wherein the disease or condition is Ceroid lipofuscinosis (Batten disease) and/or wherein the heterologous nucleic acid sequence encodes CLN4 (e.g., a polypeptide represented by UniProt Accession number Q9H3Z4 or a polypeptide comprising an amino acid sequence having at least 95% sequence identity thereto).
957. The method of any one of embodiments 431 to 434, wherein the disease or condition is Ceroid lipofuscinosis (Batten disease) and/or wherein the heterologous nucleic acid sequence encodes CLN5 (e.g., a polypeptide represented by UniProt Accession number O75503 or a polypeptide comprising an amino acid sequence having at least 95% sequence identity thereto).
958. The method of any one of embodiments 431 to 434, wherein the disease or condition is Ceroid lipofuscinosis (Batten disease) and/or wherein the heterologous nucleic acid sequence encodes CLN6 (e.g., a polypeptide represented by UniProt Accession number Q9NWW5 or a polypeptide comprising an amino acid sequence having at least 95% sequence identity thereto).
959. The method of any one of embodiments 431 to 434, wherein the disease or condition is Ceroid lipofuscinosis (Batten disease) and/or wherein the heterologous nucleic acid sequence encodes CLN7 (MFSD8) (e.g., a polypeptide represented by UniProt Accession number Q8NHS3 or a polypeptide comprising an amino acid sequence having at least 95% sequence identity thereto).
960. The method of any one of embodiments 431 to 434, wherein the disease or condition is Ceroid lipofuscinosis (Batten disease) and/or wherein the heterologous nucleic acid sequence encodes CLN8 (e.g., a polypeptide represented by UniProt Accession number Q9UBY8 or a polypeptide comprising an amino acid sequence having at least 95% sequence identity thereto).
961. The method of any one of embodiments 431 to 434, wherein the disease or condition is Ceroid lipofuscinosis (Batten disease) and/or wherein the heterologous nucleic acid sequence encodes CLN10 (cathepsin D) (e.g., a polypeptide represented by UniProt Accession number P07339 or a polypeptide comprising an amino acid sequence having at least 95% sequence identity thereto).
962. The method of any one of embodiments 431 to 434, wherein the disease or condition is Ceroid lipofuscinosis (Batten disease) and/or wherein the heterologous nucleic acid sequence encodes CLN11 (progranulin) (e.g., a polypeptide represented by UniProt Accession number P28799 or a polypeptide comprising an amino acid sequence having at least 95% sequence identity thereto).
963. The method of any one of embodiments 431 to 434, wherein the disease or condition is Ceroid lipofuscinosis (Batten disease) and/or wherein the heterologous nucleic acid sequence encodes CLN12 (ATP13A2) (e.g., a polypeptide represented by UniProt Accession number Q9NQ11 or a polypeptide comprising an amino acid sequence having at least 95% sequence identity thereto).
964. The method of any one of embodiments 431 to 434, wherein the disease or condition is Ceroid lipofuscinosis (Batten disease) and/or wherein the heterologous nucleic acid sequence encodes CLN13 (cathepsin F) (e.g., a polypeptide represented by UniProt Accession number Q9UBX1 or a polypeptide comprising an amino acid sequence having at least 95% sequence identity thereto).
965. The method of any one of embodiments 431 to 434, wherein the disease or condition is Ceroid lipofuscinosis (Batten disease) and/or wherein the heterologous nucleic acid sequence encodes CLN14 (KCTD7) (e.g., a polypeptide represented by UniProt Accession number Q96MP8 or a polypeptide comprising an amino acid sequence having at least 95% sequence identity thereto).
966. The method of any one of embodiments 431 to 434, wherein the disease or condition is Charcot-Marie-Tooth disease and/or wherein the heterologous nucleic acid sequence encodes PMP22 (e.g., a polypeptide represented by UniProt Accession number Q01453 or a polypeptide comprising an amino acid sequence having at least 95% sequence identity thereto).
967. The method of any one of embodiments 431 to 434, wherein the disease or condition is Charcot-Marie-Tooth disease and/or wherein the heterologous nucleic acid sequence encodes MPZ (e.g., a polypeptide represented by UniProt Accession number P25189 or a polypeptide comprising an amino acid sequence having at least 95% sequence identity thereto).
968. The method of any one of embodiments 431 to 434, wherein the disease or condition is Charcot-Marie-Tooth disease and/or wherein the heterologous nucleic acid sequence encodes DNM2 (e.g., a polypeptide represented by UniProt Accession number P50570 or a polypeptide comprising an amino acid sequence having at least 95% sequence identity thereto).
969. The method of any one of embodiments 431 to 434, wherein the disease or condition is Charcot-Marie-Tooth disease and/or wherein the heterologous nucleic acid sequence encodes MFN2 (e.g., a polypeptide represented by UniProt Accession number O95140 or a polypeptide comprising an amino acid sequence having at least 95% sequence identity thereto).
970. The method of any one of embodiments 431 to 434, wherein the disease or condition is Charcot-Marie-Tooth disease and/or wherein the heterologous nucleic acid sequence encodes KIF1B (e.g., a polypeptide represented by UniProt Accession number O60333 or a polypeptide comprising an amino acid sequence having at least 95% sequence identity thereto).
971. The method of any one of embodiments 431 to 434, wherein the disease or condition is Charcot-Marie-Tooth disease and/or wherein the heterologous nucleic acid sequence encodes SBF2 (e.g., a polypeptide represented by UniProt Accession number Q86WG5 or a polypeptide comprising an amino acid sequence having at least 95% sequence identity thereto).
972. The method of any one of embodiments 431 to 434, wherein the disease or condition is Charcot-Marie-Tooth disease and/or wherein the heterologous nucleic acid sequence encodes PNKP (e.g., a polypeptide represented by UniProt Accession number Q96T60 or a polypeptide comprising an amino acid sequence having at least 95% sequence identity thereto).
973. The method of any one of embodiments 431 to 434, wherein the disease or condition is Charcot-Marie-Tooth disease and/or wherein the heterologous nucleic acid sequence encodes GDAP1 (e.g., a polypeptide represented by UniProt Accession number Q8TB36 or a polypeptide comprising an amino acid sequence having at least 95% sequence identity thereto).
974. The method of any one of embodiments 431 to 434, wherein the disease or condition is Charcot-Marie-Tooth disease and/or wherein the heterologous nucleic acid sequence encodes LMNA (e.g., a polypeptide represented by UniProt Accession number P02545 or a polypeptide comprising an amino acid sequence having at least 95% sequence identity thereto).
975. The method of any one of embodiments 431 to 434, wherein the disease or condition is Charcot-Marie-Tooth disease and/or wherein the heterologous nucleic acid sequence encodes FGD4 (e.g., a polypeptide represented by UniProt Accession number Q96M96 or a polypeptide comprising an amino acid sequence having at least 95% sequence identity thereto).
976. The method of any one of embodiments 431 to 434, wherein the disease or condition is Charcot-Marie-Tooth disease and/or wherein the heterologous nucleic acid sequence encodes MTMR2 (e.g., a polypeptide represented by UniProt Accession number Q13614 or a polypeptide comprising an amino acid sequence having at least 95% sequence identity thereto).
977. The method of any one of embodiments 431 to 434, wherein the disease or condition is Chorea and/or wherein the heterologous nucleic acid sequence encodes NKX2-1 (e.g., a polypeptide represented by UniProt Accession number P43699 or a polypeptide comprising an amino acid sequence having at least 95% sequence identity thereto).
978. The method of any one of embodiments 431 to 434, wherein the disease or condition is Choreoacanthocytosis and/or wherein the heterologous nucleic acid sequence encodes VPS13A (e.g., a polypeptide represented by UniProt Accession number Q96RL7 or a polypeptide comprising an amino acid sequence having at least 95% sequence identity thereto).
979. The method of any one of embodiments 431 to 434, wherein the disease or condition is Choroideremia and/or wherein the heterologous nucleic acid sequence encodes Rab escort protein (Rep1), CHM (e.g., a polypeptide represented by UniProt Accession number P24386 or a polypeptide comprising an amino acid sequence having at least 95% sequence identity thereto).
980. The method of any one of embodiments 431 to 434, wherein the disease or condition is Cockayne syndrome B (CSB) and/or wherein the heterologous nucleic acid sequence encodes ERCC6 (e.g., a polypeptide represented by UniProt Accession number Q03468 or a polypeptide comprising an amino acid sequence having at least 95% sequence identity thereto).
981. The method of any one of embodiments 431 to 434, wherein the disease or condition is Coffin-Lowry syndrome and/or wherein the heterologous nucleic acid sequence encodes RPS6KA3 (e.g., a polypeptide represented by UniProt Accession number P51812 or a polypeptide comprising an amino acid sequence having at least 95% sequence identity thereto).
982. The method of any one of embodiments 431 to 434, wherein the disease or condition is Craniosynostosis and/or wherein the heterologous nucleic acid sequence encodes MSX2 (e.g., a polypeptide represented by UniProt Accession number P35548 or a polypeptide comprising an amino acid sequence having at least 95% sequence identity thereto).
983. The method of any one of embodiments 431 to 434, wherein the disease or condition is Craniosynostosis and/or wherein the heterologous nucleic acid sequence encodes TWIST1 (e.g., a polypeptide represented by UniProt Accession number Q15672 or a polypeptide comprising an amino acid sequence having at least 95% sequence identity thereto).
984. The method of any one of embodiments 431 to 434, wherein the disease or condition is Craniosynostosis and/or wherein the heterologous nucleic acid sequence encodes SKI (e.g., a polypeptide represented by UniProt Accession number P12755 or a polypeptide comprising an amino acid sequence having at least 95% sequence identity thereto).
985. The method of any one of embodiments 431 to 434, wherein the disease or condition is Craniosynostosis and/or wherein the heterologous nucleic acid sequence encodes SMAD6 (e.g., a polypeptide represented by UniProt Accession number O43541 or a polypeptide comprising an amino acid sequence having at least 95% sequence identity thereto).
986. The method of any one of embodiments 431 to 434, wherein the disease or condition is Creutzfeldt-Jakob disease and/or wherein the heterologous nucleic acid sequence encodes PRNP (e.g., a polypeptide represented by UniProt Accession number P04156 or a polypeptide comprising an amino acid sequence having at least 95% sequence identity thereto).
987. The method of any one of embodiments 431 to 434, wherein the disease or condition is Creutzfeldt-Jakob disease and/or wherein the heterologous nucleic acid sequence encodes HLA-DQB1 (e.g., a polypeptide represented by UniProt Accession number P01920 or a polypeptide comprising an amino acid sequence having at least 95% sequence identity thereto).
988. The method of any one of embodiments 431 to 434, wherein the disease or condition is Crigler-Najjar Syndrome (hyperbilirubinemia) and/or wherein the heterologous nucleic acid sequence encodes UDP-glucuronosyltransferase 1A1 (UGT1A1) (e.g., a polypeptide represented by UniProt Accession number P22309 or a polypeptide comprising an amino acid sequence having at least 95% sequence identity thereto).
989. The method of any one of embodiments 431 to 434, wherein the disease or condition is Cushing Syndrome and/or wherein the heterologous nucleic acid sequence encodes PRKACA (e.g., a polypeptide represented by UniProt Accession number P17612 or a polypeptide comprising an amino acid sequence having at least 95% sequence identity thereto).
990. The method of any one of embodiments 431 to 434, wherein the disease or condition is Dentatorubral atrophy (DRPLA) and/or wherein the heterologous nucleic acid sequence encodes ATN1 (e.g., a polypeptide represented by UniProt Accession number P54259 or a polypeptide comprising an amino acid sequence having at least 95% sequence identity thereto).
991. The method of any one of embodiments 431 to 434, wherein the disease or condition is Developmental and Epileptic Encephalopathy and/or wherein the heterologous nucleic acid sequence encodes ARX (e.g., a polypeptide represented by UniProt Accession number Q96QS3 or a polypeptide comprising an amino acid sequence having at least 95% sequence identity thereto).
992. The method of any one of embodiments 431 to 434, wherein the disease or condition is Developmental and Epileptic Encephalopathy and/or wherein the heterologous nucleic acid sequence encodes FGF12 (e.g., a polypeptide represented by UniProt Accession number P61328 or a polypeptide comprising an amino acid sequence having at least 95% sequence identity thereto).
993. The method of any one of embodiments 431 to 434, wherein the disease or condition is Developmental and Epileptic Encephalopathy and/or wherein the heterologous nucleic acid sequence encodes PIGP (e.g., a polypeptide represented by UniProt Accession number P57054 or a polypeptide comprising an amino acid sequence having at least 95% sequence identity thereto).
994. The method of any one of embodiments 431 to 434, wherein the disease or condition is Developmental and Epileptic Encephalopathy and/or wherein the heterologous nucleic acid sequence encodes GABRB3 (e.g., a polypeptide represented by UniProt Accession number P28472 or a polypeptide comprising an amino acid sequence having at least 95% sequence identity thereto).
995. The method of any one of embodiments 431 to 434, wherein the disease or condition is Developmental and Epileptic Encephalopathy and/or wherein the heterologous nucleic acid sequence encodes NECAP1 (e.g., a polypeptide represented by UniProt Accession number Q8NC96 or a polypeptide comprising an amino acid sequence having at least 95% sequence identity thereto).
996. The method of any one of embodiments 431 to 434, wherein the disease or condition is Developmental Dyspraxia (speech-language disorder 1 (SPCH1)) and/or wherein the heterologous nucleic acid sequence encodes FOXP2 (e.g., a polypeptide represented by UniProt Accession number O15409 or a polypeptide comprising an amino acid sequence having at least 95% sequence identity thereto).
997. The method of any one of embodiments 431 to 434, wherein the disease or condition is Dravet syndrome and/or wherein the heterologous nucleic acid sequence encodes Sodium channel protein type 1 subunit alpha (SCN1A) (e.g., a polypeptide represented by UniProt Accession number P35498 or a polypeptide comprising an amino acid sequence having at least 95% sequence identity thereto).
998. The method of any one of embodiments 431 to 434, wherein the disease or condition is Dravet syndrome and/or wherein the heterologous nucleic acid sequence encodes SCN1B (e.g., a polypeptide represented by UniProt Accession number Q07699 or a polypeptide comprising an amino acid sequence having at least 95% sequence identity thereto).
999. The method of any one of embodiments 431 to 434, wherein the disease or condition is Dravet syndrome and/or wherein the heterologous nucleic acid sequence encodes SCN2A (e.g., a polypeptide represented by UniProt Accession number Q99250 or a polypeptide comprising an amino acid sequence having at least 95% sequence identity thereto).
1000. The method of any one of embodiments 431 to 434, wherein the disease or condition is Dravet syndrome and/or wherein the heterologous nucleic acid sequence encodes GABA receptor subunit gamma-2 (GABRG2) (e.g., a polypeptide represented by UniProt Accession number P18507 or a polypeptide comprising an amino acid sequence having at least 95% sequence identity thereto).
1001. The method of any one of embodiments 431 to 434, wherein the disease or condition is Dysautonomia (Day syndrome) and/or wherein the heterologous nucleic acid sequence encodes ELP1 (e.g., a polypeptide represented by UniProt Accession number O95163 or a polypeptide comprising an amino acid sequence having at least 95% sequence identity thereto).
1002. The method of any one of embodiments 431 to 434, wherein the disease or condition is Dystonia and/or wherein the heterologous nucleic acid sequence encodes GCH1 (e.g., a polypeptide represented by UniProt Accession number P30793 or a polypeptide comprising an amino acid sequence having at least 95% sequence identity thereto).
1003. The method of any one of embodiments 431 to 434, wherein the disease or condition is Dystonia and/or wherein the heterologous nucleic acid sequence encodes TOR1A (e.g., a polypeptide represented by UniProt Accession number O14656 or a polypeptide comprising an amino acid sequence having at least 95% sequence identity thereto).
1004. The method of any one of embodiments 431 to 434, wherein the disease or condition is Dystonia and/or wherein the heterologous nucleic acid sequence encodes SGCE (e.g., a polypeptide represented by UniProt Accession number O43556 or a polypeptide comprising an amino acid sequence having at least 95% sequence identity thereto).
1005. The method of any one of embodiments 431 to 434, wherein the disease or condition is Dystonia and/or wherein the heterologous nucleic acid sequence encodes TUBB4A (e.g., a polypeptide represented by UniProt Accession number P04350 or a polypeptide comprising an amino acid sequence having at least 95% sequence identity thereto).
1006. The method of any one of embodiments 431 to 434, wherein the disease or condition is Encephalocele and/or wherein the heterologous nucleic acid sequence encodes COL18A1 (e.g., a polypeptide represented by UniProt Accession number P39060 or a polypeptide comprising an amino acid sequence having at least 95% sequence identity thereto).
1007. The method of any one of embodiments 431 to 434, wherein the disease or condition is Epilepsy disorder and/or wherein the heterologous nucleic acid sequence encodes GRIN2A (e.g., a polypeptide represented by UniProt Accession number Q12879 or a polypeptide comprising an amino acid sequence having at least 95% sequence identity thereto).
1008. The method of any one of embodiments 431 to 434, wherein the disease or condition is Epilepsy disorder and/or wherein the heterologous nucleic acid sequence encodes CSTB (e.g., a polypeptide represented by UniProt Accession number P04080 or a polypeptide comprising an amino acid sequence having at least 95% sequence identity thereto).
1009. The method of any one of embodiments 431 to 434, wherein the disease or condition is Epilepsy disorder and/or wherein the heterologous nucleic acid sequence encodes STARD7 (e.g., a polypeptide represented by UniProt Accession number Q9NQZ5 or a polypeptide comprising an amino acid sequence having at least 95% sequence identity thereto).
1010. The method of any one of embodiments 431 to 434, wherein the disease or condition is Epilepsy disorder and/or wherein the heterologous nucleic acid sequence encodes DEPDC5 (e.g., a polypeptide represented by UniProt Accession number O75140 or a polypeptide comprising an amino acid sequence having at least 95% sequence identity thereto).
1011. The method of any one of embodiments 431 to 434, wherein the disease or condition is Epilepsy disorder and/or wherein the heterologous nucleic acid sequence encodes PCDH19 (e.g., a polypeptide represented by UniProt Accession number Q8TAB3 or a polypeptide comprising an amino acid sequence having at least 95% sequence identity thereto).
1012. The method of any one of embodiments 431 to 434, wherein the disease or condition is Essential tremor and/or wherein the heterologous nucleic acid sequence encodes DRD3 (e.g., a polypeptide represented by UniProt Accession number P35462 or a polypeptide comprising an amino acid sequence having at least 95% sequence identity thereto).
1013. The method of any one of embodiments 431 to 434, wherein the disease or condition is Essential tremor and/or wherein the heterologous nucleic acid sequence encodes NOTCH2NLC (e.g., a polypeptide represented by UniProt Accession number PODPK4 or a polypeptide comprising an amino acid sequence having at least 95% sequence identity thereto).
1014. The method of any one of embodiments 431 to 434, wherein the disease or condition is Essential tremor and/or wherein the heterologous nucleic acid sequence encodes FUS (e.g., a polypeptide represented by UniProt Accession number P35637 or a polypeptide comprising an amino acid sequence having at least 95% sequence identity thereto).
1015. The method of any one of embodiments 431 to 434, wherein the disease or condition is Fabry disease and/or wherein the heterologous nucleic acid sequence encodes alpha-galactosidase A (GLA) (e.g., a polypeptide represented by UniProt Accession number P06280 or a polypeptide comprising an amino acid sequence having at least 95% sequence identity thereto).
1016. The method of any one of embodiments 431 to 434, wherein the disease or condition is Farber disease (ceramidase deficiency) and/or wherein the heterologous nucleic acid sequence encodes ASAH1 (e.g., a polypeptide represented by UniProt Accession number Q13510 or a polypeptide comprising an amino acid sequence having at least 95% sequence identity thereto).
1017. The method of any one of embodiments 431 to 434, wherein the disease or condition is Fahr disease and/or wherein the heterologous nucleic acid sequence encodes SLC20A2 (e.g., a polypeptide represented by UniProt Accession number Q08357 or a polypeptide comprising an amino acid sequence having at least 95% sequence identity thereto).
1018. The method of any one of embodiments 431 to 434, wherein the disease or condition is Febrile Seizure and/or wherein the heterologous nucleic acid sequence encodes GABRG2 (e.g., a polypeptide represented by UniProt Accession number P18507 or a polypeptide comprising an amino acid sequence having at least 95% sequence identity thereto).
1019. The method of any one of embodiments 431 to 434, wherein the disease or condition is Febrile Seizure and/or wherein the heterologous nucleic acid sequence encodes ADGRV1 (e.g., a polypeptide represented by UniProt Accession number Q8WXG9 or a polypeptide comprising an amino acid sequence having at least 95% sequence identity thereto).
1020. The method of any one of embodiments 431 to 434, wherein the disease or condition is Febrile Seizure and/or wherein the heterologous nucleic acid sequence encodes CPA6 (e.g., a polypeptide represented by UniProt Accession number P11509 or a polypeptide comprising an amino acid sequence having at least 95% sequence identity thereto).
1021. The method of any one of embodiments 431 to 434, wherein the disease or condition is Febrile Seizure and/or wherein the heterologous nucleic acid sequence encodes SCN1A (e.g., a polypeptide represented by UniProt Accession number P35498 or a polypeptide comprising an amino acid sequence having at least 95% sequence identity thereto).
1022. The method of any one of embodiments 431 to 434, wherein the disease or condition is Friedreich's ataxia and/or wherein the heterologous nucleic acid sequence encodes Frataxin (FXN) (e.g., a polypeptide represented by UniProt Accession number Q16595 or a polypeptide comprising an amino acid sequence having at least 95% sequence identity thereto).
1023. The method of any one of embodiments 431 to 434, wherein the disease or condition is Frontotemporal dementia and/or wherein the heterologous nucleic acid sequence encodes Progranulin (GRN) (e.g., a polypeptide represented by UniProt Accession number P28799 or a polypeptide comprising an amino acid sequence having at least 95% sequence identity thereto).
1024. The method of any one of embodiments 431 to 434, wherein the disease or condition is Frontotemporal dementia and/or wherein the heterologous nucleic acid sequence encodes MAPT (tau) (e.g., a polypeptide represented by UniProt Accession number P10636 or a polypeptide comprising an amino acid sequence having at least 95% sequence identity thereto).
1025. The method of any one of embodiments 431 to 434, wherein the disease or condition is Frontotemporal dementia and/or wherein the heterologous nucleic acid sequence encodes PSEN1 (e.g., a polypeptide represented by UniProt Accession number A0A024R6A3 or a polypeptide comprising an amino acid sequence having at least 95% sequence identity thereto).
1026. The method of any one of embodiments 431 to 434, wherein the disease or condition is Fucosidosis and/or wherein the heterologous nucleic acid sequence encodes alpha-L-fucosidase (FUCA1) (e.g., a polypeptide represented by UniProt Accession number P04066 or a polypeptide comprising an amino acid sequence having at least 95% sequence identity thereto).
1027. The method of any one of embodiments 431 to 434, wherein the disease or condition is Fundus albipunctatus and/or wherein the heterologous nucleic acid sequence encodes RLBP1 (e.g., a polypeptide represented by UniProt Accession number P12271 or a polypeptide comprising an amino acid sequence having at least 95% sequence identity thereto).
1028. The method of any one of embodiments 431 to 434, wherein the disease or condition is Gaucher disease (e.g., types I, II or Ill) and/or wherein the heterologous nucleic acid sequence encodes Glucocerebrosidase (GBA1) (e.g., a polypeptide represented by UniProt Accession number P04062 or a polypeptide comprising an amino acid sequence having at least 95% sequence identity thereto).
1029. The method of any one of embodiments 431 to 434, wherein the disease or condition is Generalized gangliosidose (e.g., GM1, GM2 or GM3) and/or wherein the heterologous nucleic acid sequence encodes GLB1 (e.g., a polypeptide represented by UniProt Accession number P16278 or a polypeptide comprising an amino acid sequence having at least 95% sequence identity thereto).
1030. The method of any one of embodiments 431 to 434, wherein the disease or condition is Gerstmann-Straussler-Scheinker disease and/or wherein the heterologous nucleic acid sequence encodes PRNP (e.g., a polypeptide represented by UniProt Accession number P04156 or a polypeptide comprising an amino acid sequence having at least 95% sequence identity thereto).
1031. The method of any one of embodiments 431 to 434, wherein the disease or condition is Giant axonal neuropathy and/or wherein the heterologous nucleic acid sequence encodes Gigaxonin (GAN) (e.g., a polypeptide represented by UniProt Accession number Q9H2C0 or a polypeptide comprising an amino acid sequence having at least 95% sequence identity thereto).
1032. The method of any one of embodiments 431 to 434, wherein the disease or condition is Glycogen storage disease II (Pompe disease or Acid Maltase Deficiency) and/or wherein the heterologous nucleic acid sequence encodes Acid maltase, lysosomal alpha-glucosidase (LYAG, GAA) (e.g., a polypeptide represented by UniProt Accession number P10253 or a polypeptide comprising an amino acid sequence having at least 95% sequence identity thereto).
1033. The method of any one of embodiments 431 to 434, wherein the disease or condition is Guillain-Barre syndrome and/or wherein the heterologous nucleic acid sequence encodes PMP22 (e.g., a polypeptide represented by UniProt Accession number Q01453 or a polypeptide comprising an amino acid sequence having at least 95% sequence identity thereto).
1034. The method of any one of embodiments 431 to 434, wherein the disease or condition is Chronic Inflammatory Demyelinating Polyneuropathy (CIDP) and/or wherein the heterologous nucleic acid sequence encodes PMP22 (e.g., a polypeptide represented by UniProt Accession number Q01453 or a polypeptide comprising an amino acid sequence having at least 95% sequence identity thereto).
1035. The method of any one of embodiments 431 to 434, wherein the disease or condition is Hallervorden-Spatz disease (PKAN or NBIA1) and/or wherein the heterologous nucleic acid sequence encodes PANK2 (e.g., a polypeptide represented by UniProt Accession number Q9BZ23 or a polypeptide comprising an amino acid sequence having at least 95% sequence identity thereto).
1036. The method of any one of embodiments 431 to 434, wherein the disease or condition is Hemiplegia Alterans and/or wherein the heterologous nucleic acid sequence encodes ATP1A2 (e.g., a polypeptide represented by UniProt Accession number P50993 or a polypeptide comprising an amino acid sequence having at least 95% sequence identity thereto).
1037. The method of any one of embodiments 431 to 434, wherein the disease or condition is Hemiplegia Alterans and/or wherein the heterologous nucleic acid sequence encodes ATP1A3 (e.g., a polypeptide represented by UniProt Accession number P13637 or a polypeptide comprising an amino acid sequence having at least 95% sequence identity thereto).
1038. The method of any one of embodiments 431 to 434, wherein the disease or condition is Hereditary Neuropathy and/or wherein the heterologous nucleic acid sequence encodes WNK1 (e.g., a polypeptide represented by UniProt Accession number Q9H4A3 or a polypeptide comprising an amino acid sequence having at least 95% sequence identity thereto).
1039. The method of any one of embodiments 431 to 434, wherein the disease or condition is Hereditary Neuropathy and/or wherein the heterologous nucleic acid sequence encodes MFN2 (e.g., a polypeptide represented by UniProt Accession number O95140 or a polypeptide comprising an amino acid sequence having at least 95% sequence identity thereto).
1040. The method of any one of embodiments 431 to 434, wherein the disease or condition is Hereditary Neuropathy and/or wherein the heterologous nucleic acid sequence encodes HK1 (e.g., a polypeptide represented by UniProt Accession number P19367 or a polypeptide comprising an amino acid sequence having at least 95% sequence identity thereto).
1041. The method of any one of embodiments 431 to 434, wherein the disease or condition is Hereditary Neuropathy and/or wherein the heterologous nucleic acid sequence encodes TFG (e.g., a polypeptide represented by UniProt Accession number Q92734 or a polypeptide comprising an amino acid sequence having at least 95% sequence identity thereto).
1042. The method of any one of embodiments 431 to 434, wherein the disease or condition is Hereditary Neuropathy and/or wherein the heterologous nucleic acid sequence encodes SPTLC1 (e.g., a polypeptide represented by UniProt Accession number O15269 or a polypeptide comprising an amino acid sequence having at least 95% sequence identity thereto).
1043. The method of any one of embodiments 431 to 434, wherein the disease or condition is Heredopathia Atactica Polyneuritiformis (Refsum disease) and/or wherein the heterologous nucleic acid sequence encodes PHYH (e.g., a polypeptide represented by UniProt Accession number O14832 or a polypeptide comprising an amino acid sequence having at least 95% sequence identity thereto).
1044. The method of any one of embodiments 431 to 434, wherein the disease or condition is Holoprosencephaly and/or wherein the heterologous nucleic acid sequence encodes GLI2 (e.g., a polypeptide represented by UniProt Accession number P10070 or a polypeptide comprising an amino acid sequence having at least 95% sequence identity thereto).
1045. The method of any one of embodiments 431 to 434, wherein the disease or condition is Holoprosencephaly and/or wherein the heterologous nucleic acid sequence encodes TGIF1 (e.g., a polypeptide represented by UniProt Accession number Q15583 or a polypeptide comprising an amino acid sequence having at least 95% sequence identity thereto).
1046. The method of any one of embodiments 431 to 434, wherein the disease or condition is Holoprosencephaly and/or wherein the heterologous nucleic acid sequence encodes ZIC2 (e.g., a polypeptide represented by UniProt Accession number O95409 or a polypeptide comprising an amino acid sequence having at least 95% sequence identity thereto).
1047. The method of any one of embodiments 431 to 434, wherein the disease or condition is Holoprosencephaly and/or wherein the heterologous nucleic acid sequence encodes PTCH1 (e.g., a polypeptide represented by UniProt Accession number Q13635 or a polypeptide comprising an amino acid sequence having at least 95% sequence identity thereto).
1048. The method of any one of embodiments 431 to 434, wherein the disease or condition is Holoprosencephaly and/or wherein the heterologous nucleic acid sequence encodes SHH (e.g., a polypeptide represented by UniProt Accession number Q15465 or a polypeptide comprising an amino acid sequence having at least 95% sequence identity thereto).
1049. The method of any one of embodiments 431 to 434, wherein the disease or condition is Huntington's disease and/or wherein the heterologous nucleic acid sequence encodes HTT (e.g., a polypeptide represented by UniProt Accession number P42858 or a polypeptide comprising an amino acid sequence having at least 95% sequence identity thereto).
1050. The method of any one of embodiments 431 to 434, wherein the disease or condition is Hydrocephalus disorder and/or wherein the heterologous nucleic acid sequence encodes CCDC88C (e.g., a polypeptide represented by UniProt Accession number Q9P219 or a polypeptide comprising an amino acid sequence having at least 95% sequence identity thereto).
1051. The method of any one of embodiments 431 to 434, wherein the disease or condition is Hydrocephalus disorder and/or wherein the heterologous nucleic acid sequence encodes WDR81 (e.g., a polypeptide represented by UniProt Accession number Q562E7 or a polypeptide comprising an amino acid sequence having at least 95% sequence identity thereto).
1052. The method of any one of embodiments 431 to 434, wherein the disease or condition is Hydrocephalus disorder and/or wherein the heterologous nucleic acid sequence encodes TRIM71 (e.g., a polypeptide represented by UniProt Accession number Q2Q1W2 or a polypeptide comprising an amino acid sequence having at least 95% sequence identity thereto).
1053. The method of any one of embodiments 431 to 434, wherein the disease or condition is Hydrocephalus disorder and/or wherein the heterologous nucleic acid sequence encodes MPDZ (e.g., a polypeptide represented by UniProt Accession number O75970 or a polypeptide comprising an amino acid sequence having at least 95% sequence identity thereto).
1054. The method of any one of embodiments 431 to 434, wherein the disease or condition is Incontinentia Pigmenti and/or wherein the heterologous nucleic acid sequence encodes IKBKG (e.g., a polypeptide represented by UniProt Accession number Q9Y6K9 or a polypeptide comprising an amino acid sequence having at least 95% sequence identity thereto).
1055. The method of any one of embodiments 431 to 434, wherein the disease or condition is Infantile Hypotonia and/or wherein the heterologous nucleic acid sequence encodes NALCN (e.g., a polypeptide represented by UniProt Accession number Q8IZF0 or a polypeptide comprising an amino acid sequence having at least 95% sequence identity thereto).
1056. The method of any one of embodiments 431 to 434, wherein the disease or condition is Infantile Hypotonia and/or wherein the heterologous nucleic acid sequence encodes TBCK (e.g., a polypeptide represented by UniProt Accession number Q8TEA7 or a polypeptide comprising an amino acid sequence having at least 95% sequence identity thereto).
1057. The method of any one of embodiments 431 to 434, wherein the disease or condition is Infantile Hypotonia and/or wherein the heterologous nucleic acid sequence encodes CCDC174 (e.g., a polypeptide represented by UniProt Accession number Q6PII3 or a polypeptide comprising an amino acid sequence having at least 95% sequence identity thereto).
1058. The method of any one of embodiments 431 to 434, wherein the disease or condition is Infantile Hypotonia and/or wherein the heterologous nucleic acid sequence encodes UNC80 (e.g., a polypeptide represented by UniProt Accession number Q8N2C7 or a polypeptide comprising an amino acid sequence having at least 95% sequence identity thereto).
1059. The method of any one of embodiments 431 to 434, wherein the disease or condition is Infantile Neuroaxonal Dystrophy and/or wherein the heterologous nucleic acid sequence encodes PLA2G6 (e.g., a polypeptide represented by UniProt Accession number O60733 or a polypeptide comprising an amino acid sequence having at least 95% sequence identity thereto).
1060. The method of any one of embodiments 431 to 434, wherein the disease or condition is Infantile Phytanic Acid Storage Disease (PBD1B) and/or wherein the heterologous nucleic acid sequence encodes PEX1 (e.g., a polypeptide represented by UniProt Accession number O43933 or a polypeptide comprising an amino acid sequence having at least 95% sequence identity thereto).
1061. The method of any one of embodiments 431 to 434, wherein the disease or condition is Joubert Syndrome and/or wherein the heterologous nucleic acid sequence encodes INPP5E (e.g., a polypeptide represented by UniProt Accession number Q9NRR6 or a polypeptide comprising an amino acid sequence having at least 95% sequence identity thereto).
1062. The method of any one of embodiments 431 to 434, wherein the disease or condition is Kennedy Disease and/or wherein the heterologous nucleic acid sequence encodes Androgen receptor (AR) (e.g., a polypeptide represented by UniProt Accession number P10275 or a polypeptide comprising an amino acid sequence having at least 95% sequence identity thereto).
1063. The method of any one of embodiments 431 to 434, wherein the disease or condition is Klippel-Feil Syndrome and/or wherein the heterologous nucleic acid sequence encodes GDF6 (e.g., a polypeptide represented by UniProt Accession number Q6KF10 or a polypeptide comprising an amino acid sequence having at least 95% sequence identity thereto).
1064. The method of any one of embodiments 431 to 434, wherein the disease or condition is Krabbe disease (GALC deficiency) and/or wherein the heterologous nucleic acid sequence encodes GALC (e.g., a polypeptide represented by UniProt Accession number P54803 or a polypeptide comprising an amino acid sequence having at least 95% sequence identity thereto).
1065. The method of any one of embodiments 431 to 434, wherein the disease or condition is Lambert-Eaton Myasthenic Syndrome and/or wherein the heterologous nucleic acid sequence encodes CACNA1A (e.g., a polypeptide represented by UniProt Accession number O00555 or a polypeptide comprising an amino acid sequence having at least 95% sequence identity thereto).
1066. The method of any one of embodiments 431 to 434, wherein the disease or condition is Lambert-Eaton Myasthenic Syndrome and/or wherein the heterologous nucleic acid sequence encodes CACNB2 (e.g., a polypeptide represented by UniProt Accession number Q13936 or a polypeptide comprising an amino acid sequence having at least 95% sequence identity thereto).
1067. The method of any one of embodiments 431 to 434, wherein the disease or condition is Landau-Kleffner Syndrome and/or wherein the heterologous nucleic acid sequence encodes GRIN2A (e.g., a polypeptide represented by UniProt Accession number Q12879 or a polypeptide comprising an amino acid sequence having at least 95% sequence identity thereto).
1068. The method of any one of embodiments 431 to 434, wherein the disease or condition is Late infantile neuronal lipofuscinosis (CLN2) and/or wherein the heterologous nucleic acid sequence encodes TPP1 (e.g., a polypeptide represented by UniProt Accession number O14773 or a polypeptide comprising an amino acid sequence having at least 95% sequence identity thereto).
1069. The method of any one of embodiments 431 to 434, wherein the disease or condition is Lesch-Nyhan Syndrome and/or wherein the heterologous nucleic acid sequence encodes HPRT1 (e.g., a polypeptide represented by UniProt Accession number P00492 or a polypeptide comprising an amino acid sequence having at least 95% sequence identity thereto).
1070. The method of any one of embodiments 431 to 434, wherein the disease or condition is Leber congenital amaurosis (retinal blindness) and/or wherein the heterologous nucleic acid sequence encodes Retinal guanylyl cyclase 1 (GUCY2D) (e.g., a polypeptide represented by UniProt Accession number Q02846 or a polypeptide comprising an amino acid sequence having at least 95% sequence identity thereto).
1071. The method of any one of embodiments 431 to 434, wherein the disease or condition is Leber congenital amaurosis (retinal blindness) and/or wherein the heterologous nucleic acid sequence encodes Retinoid isomerohydrolase (RPE65) (e.g., a polypeptide represented by UniProt Accession number Q16518 or a polypeptide comprising an amino acid sequence having at least 95% sequence identity thereto).
1072. The method of any one of embodiments 431 to 434, wherein the disease or condition is Leber congenital amaurosis (retinal blindness) and/or wherein the heterologous nucleic acid sequence encodes Centrosomal protein of 290 kDa (CEP290) (e.g., a polypeptide represented by UniProt Accession number 015078 or a polypeptide comprising an amino acid sequence having at least 95% sequence identity thereto).
1073. The method of any one of embodiments 431 to 434, wherein the disease or condition is Leber congenital amaurosis (retinal blindness) and/or wherein the heterologous nucleic acid sequence encodes Protein crumbs homolog 1 (CRB1) (e.g., a polypeptide represented by UniProt Accession number P82279 or a polypeptide comprising an amino acid sequence having at least 95% sequence identity thereto).
1074. The method of any one of embodiments 431 to 434, wherein the disease or condition is Leber's hereditary optical neuropathy and/or wherein the heterologous nucleic acid sequence encodes NADH-ubiquinone oxidoreductase chain 4 (ND4) (e.g., a polypeptide represented by UniProt Accession number P03905 or a polypeptide comprising an amino acid sequence having at least 95% sequence identity thereto).
1075. The method of any one of embodiments 431 to 434, wherein the disease or condition is Leukodystrophy and/or wherein the heterologous nucleic acid sequence encodes ARSA (e.g., a polypeptide represented by UniProt Accession number P15289 or a polypeptide comprising an amino acid sequence having at least 95% sequence identity thereto).
1076. The method of any one of embodiments 431 to 434, wherein the disease or condition is Levine-Critchley Syndrome (choreoacanthocytosis) and/or wherein the heterologous nucleic acid sequence encodes VPS13A (e.g., a polypeptide represented by UniProt Accession number Q96RL7 or a polypeptide comprising an amino acid sequence having at least 95% sequence identity thereto).
1077. The method of any one of embodiments 431 to 434, wherein the disease or condition is Lewy body dementia and/or wherein the heterologous nucleic acid sequence encodes SNCA (e.g., a polypeptide represented by UniProt Accession number P37840 or a polypeptide comprising an amino acid sequence having at least 95% sequence identity thereto).
1078. The method of any one of embodiments 431 to 434, wherein the disease or condition is Lewy body dementia and/or wherein the heterologous nucleic acid sequence encodes SNCB (e.g., a polypeptide represented by UniProt Accession number Q16143 or a polypeptide comprising an amino acid sequence having at least 95% sequence identity thereto).
1079. The method of any one of embodiments 431 to 434, wherein the disease or condition is Lipoid Proteinosis (Urbach-Wiethe disease) and/or wherein the heterologous nucleic acid sequence encodes ECM1 (e.g., a polypeptide represented by UniProt Accession number Q16610 or a polypeptide comprising an amino acid sequence having at least 95% sequence identity thereto).
1080. The method of any one of embodiments 431 to 434, wherein the disease or condition is Lissencephaly and/or wherein the heterologous nucleic acid sequence encodes PAFAH1B1 (e.g., a polypeptide represented by UniProt Accession number Q9PTR5 or a polypeptide comprising an amino acid sequence having at least 95% sequence identity thereto).
1081. The method of any one of embodiments 431 to 434, wherein the disease or condition is Lissencephaly and/or wherein the heterologous nucleic acid sequence encodes NDE1 (e.g., a polypeptide represented by UniProt Accession number Q9NXR1 or a polypeptide comprising an amino acid sequence having at least 95% sequence identity thereto).
1082. The method of any one of embodiments 431 to 434, wherein the disease or condition is Lissencephaly and/or wherein the heterologous nucleic acid sequence encodes TUBA1A (e.g., a polypeptide represented by UniProt Accession number Q71U36 or a polypeptide comprising an amino acid sequence having at least 95% sequence identity thereto).
1083. The method of any one of embodiments 431 to 434, wherein the disease or condition is Lissencephaly and/or wherein the heterologous nucleic acid sequence encodes LAMB1 (e.g., a polypeptide represented by UniProt Accession number LAMB1 or a polypeptide comprising an amino acid sequence having at least 95% sequence identity thereto).
1084. The method of any one of embodiments 431 to 434, wherein the disease or condition is Lissencephaly and/or wherein the heterologous nucleic acid sequence encodes KATNB1 (e.g., a polypeptide represented by UniProt Accession number Q9BVA0 or a polypeptide comprising an amino acid sequence having at least 95% sequence identity thereto).
1085. The method of any one of embodiments 431 to 434, wherein the disease or condition is Lissencephaly and/or wherein the heterologous nucleic acid sequence encodes RELN (e.g., a polypeptide represented by UniProt Accession number P78509 or a polypeptide comprising an amino acid sequence having at least 95% sequence identity thereto).
1086. The method of any one of embodiments 431 to 434, wherein the disease or condition is Macrocephaly/Megalencephaly and/or wherein the heterologous nucleic acid sequence encodes TBC1 D7 (e.g., a polypeptide represented by UniProt Accession number Q9P0N9 or a polypeptide comprising an amino acid sequence having at least 95% sequence identity thereto).
1087. The method of any one of embodiments 431 to 434, wherein the disease or condition is Menkes Disease and/or wherein the heterologous nucleic acid sequence encodes ATP7A (e.g., a polypeptide represented by UniProt Accession number Q04656 or a polypeptide comprising an amino acid sequence having at least 95% sequence identity thereto).
1088. The method of any one of embodiments 431 to 434, wherein the disease or condition is Metachromatic Leukodystrophy (MLD) and/or wherein the heterologous nucleic acid sequence encodes Arylsulfatase A (ARSA) (e.g., a polypeptide represented by UniProt Accession number P15289 or a polypeptide comprising an amino acid sequence having at least 95% sequence identity thereto).
1089. The method of any one of embodiments 431 to 434, wherein the disease or condition is Microcephaly and/or wherein the heterologous nucleic acid sequence encodes KIF11 (e.g., a polypeptide represented by UniProt Accession number P52732 or a polypeptide comprising an amino acid sequence having at least 95% sequence identity thereto).
1090. The method of any one of embodiments 431 to 434, wherein the disease or condition is Microcephaly and/or wherein the heterologous nucleic acid sequence encodes MCPH1 (e.g., a polypeptide represented by UniProt Accession number Q8NEMO or a polypeptide comprising an amino acid sequence having at least 95% sequence identity thereto).
1091. The method of any one of embodiments 431 to 434, wherein the disease or condition is Microcephaly and/or wherein the heterologous nucleic acid sequence encodes SLC25A19 (e.g., a polypeptide represented by UniProt Accession number Q9HC21 or a polypeptide comprising an amino acid sequence having at least 95% sequence identity thereto).
1092. The method of any one of embodiments 431 to 434, wherein the disease or condition is Migraine, familial hemiplegic and/or wherein the heterologous nucleic acid sequence encodes CACNA1A (e.g., a polypeptide represented by UniProt Accession number O00555 or a polypeptide comprising an amino acid sequence having at least 95% sequence identity thereto).
1093. The method of any one of embodiments 431 to 434, wherein the disease or condition is Migraine, familial hemiplegic and/or wherein the heterologous nucleic acid sequence encodes ATP1A2 (e.g., a polypeptide represented by UniProt Accession number P50993 or a polypeptide comprising an amino acid sequence having at least 95% sequence identity thereto).
1094. The method of any one of embodiments 431 to 434, wherein the disease or condition is Migraine, familial hemiplegic and/or wherein the heterologous nucleic acid sequence encodes SCN1A (e.g., a polypeptide represented by UniProt Accession number P35498 or a polypeptide comprising an amino acid sequence having at least 95% sequence identity thereto).
1095. The method of any one of embodiments 431 to 434, wherein the disease or condition is Mitochondrial DNA depletion syndrome and/or wherein the heterologous nucleic acid sequence encodes RRM2B (e.g., a polypeptide represented by UniProt Accession number Q7LG56 or a polypeptide comprising an amino acid sequence having at least 95% sequence identity thereto).
1096. The method of any one of embodiments 431 to 434, wherein the disease or condition is Mitochondrial DNA depletion syndrome and/or wherein the heterologous nucleic acid sequence encodes DGUOK (e.g., a polypeptide represented by UniProt Accession number Q16854 or a polypeptide comprising an amino acid sequence having at least 95% sequence identity thereto).
1097. The method of any one of embodiments 431 to 434, wherein the disease or condition is Mitochondrial DNA depletion syndrome and/or wherein the heterologous nucleic acid sequence encodes POLG (e.g., a polypeptide represented by UniProt Accession number P54098 or a polypeptide comprising an amino acid sequence having at least 95% sequence identity thereto).
1098. The method of any one of embodiments 431 to 434, wherein the disease or condition is Mitochondrial DNA depletion syndrome and/or wherein the heterologous nucleic acid sequence encodes TYMP (e.g., a polypeptide represented by UniProt Accession number P19971 or a polypeptide comprising an amino acid sequence having at least 95% sequence identity thereto).
1099. The method of any one of embodiments 431 to 434, wherein the disease or condition is Mitochondrial DNA depletion syndrome and/or wherein the heterologous nucleic acid sequence encodes TK2 (e.g., a polypeptide represented by UniProt Accession number O00142 or a polypeptide comprising an amino acid sequence having at least 95% sequence identity thereto).
1100. The method of any one of embodiments 431 to 434, wherein the disease or condition is Morvan disease and/or wherein the heterologous nucleic acid sequence encodes WNK1 (e.g., a polypeptide represented by UniProt Accession number Q9H4A3 or a polypeptide comprising an amino acid sequence having at least 95% sequence identity thereto).
1101. The method of any one of embodiments 431 to 434, wherein the disease or condition is Mucolipidosis and/or wherein the heterologous nucleic acid sequence encodes GNPTAB (e.g., a polypeptide represented by UniProt Accession number Q3T906 or a polypeptide comprising an amino acid sequence having at least 95% sequence identity thereto).
1102. The method of any one of embodiments 431 to 434, wherein the disease or condition is Mucolipidosis and/or wherein the heterologous nucleic acid sequence encodes MCOLN1 (e.g., a polypeptide represented by UniProt Accession number Q9GZU1 or a polypeptide comprising an amino acid sequence having at least 95% sequence identity thereto).
1103. The method of any one of embodiments 431 to 434, wherein the disease or condition is Mucopolysaccharidosis Type I (MPS 1) (Hurler syndrome) and/or wherein the heterologous nucleic acid sequence encodes alpha-L-iduronidase (IDUA) (e.g., a polypeptide represented by UniProt Accession number P35475 or a polypeptide comprising an amino acid sequence having at least 95% sequence identity thereto).
1104. The method of any one of embodiments 431 to 434, wherein the disease or condition is MPS II (Hunter syndrome) and/or wherein the heterologous nucleic acid sequence encodes iduronate-2-sulfatase (IDS) (e.g., a polypeptide represented by UniProt Accession number P22304 or a polypeptide comprising an amino acid sequence having at least 95% sequence identity thereto).
1105. The method of any one of embodiments 431 to 434, wherein the disease or condition is MPS IIIa (Sanfilippo Type A syndrome) and/or wherein the heterologous nucleic acid sequence encodes heparan sulfate sulfatase (HSS) or N-sulfoglucosamine sulfohydrolase (SGSH) (e.g., a polypeptide represented by UniProt Accession number P51688 or a polypeptide comprising an amino acid sequence having at least 95% sequence identity thereto).
1106. The method of any one of embodiments 431 to 434, wherein the disease or condition is MPS IIIB (Sanfilippo Type B syndrome) and/or wherein the heterologous nucleic acid sequence encodes N-acetyl-alpha-D-glucosaminidase (NAGLU) (e.g., a polypeptide represented by UniProt Accession number P54802 or a polypeptide comprising an amino acid sequence having at least 95% sequence identity thereto).
1107. The method of any one of embodiments 431 to 434, wherein the disease or condition is MPS VI (Maroteaux-Lamy syndrome) and/or wherein the heterologous nucleic acid sequence encodes arylsulfatase B (ARSB) (e.g., a polypeptide represented by UniProt Accession number P15848 or a polypeptide comprising an amino acid sequence having at least 95% sequence identity thereto).
1108. The method of any one of embodiments 431 to 434, wherein the disease or condition is MPS IV A (Morquio syndrome type A) and/or wherein the heterologous nucleic acid sequence encodes N-acetylgalactosamine-6-sulfatase (GALNS) (e.g., a polypeptide represented by UniProt Accession number P34059 or a polypeptide comprising an amino acid sequence having at least 95% sequence identity thereto).
1109. The method of any one of embodiments 431 to 434, wherein the disease or condition is MPS IV B (Morquio syndrome type B) and/or wherein the heterologous nucleic acid sequence encodes Beta-galactosidase 1 (GLB1) (e.g., a polypeptide represented by UniProt Accession number P16278 or a polypeptide comprising an amino acid sequence having at least 95% sequence identity thereto).
1110. The method of any one of embodiments 431 to 434, wherein the disease or condition is MPS VII (Sly syndrome) and/or wherein the heterologous nucleic acid sequence encodes beta-glucuronidase (e.g., a polypeptide represented by UniProt Accession number P08236 or a polypeptide comprising an amino acid sequence having at least 95% sequence identity thereto).
1111. The method of any one of embodiments 431 to 434, wherein the disease or condition is MPS VIII and/or wherein the heterologous nucleic acid sequence encodes glucosamine-6-sulfate sulfatase (e.g., a polypeptide represented by UniProt Accession number P15586 or a polypeptide comprising an amino acid sequence having at least 95% sequence identity thereto).
1112. The method of any one of embodiments 431 to 434, wherein the disease or condition is MPS IX and/or wherein the heterologous nucleic acid sequence encodes Hyaluronidase-1 (HYAL1) (e.g., a polypeptide represented by UniProt Accession number Q12794 or a polypeptide comprising an amino acid sequence having at least 95% sequence identity thereto).
1113. The method of any one of embodiments 431 to 434, wherein the disease or condition is Multiple Sclerosis and/or wherein the heterologous nucleic acid sequence encodes PDCD1 (e.g., a polypeptide represented by UniProt Accession number Q15116 or a polypeptide comprising an amino acid sequence having at least 95% sequence identity thereto).
1114. The method of any one of embodiments 431 to 434, wherein the disease or condition is Multiple system atrophy and/or wherein the heterologous nucleic acid sequence encodes COQ2 (e.g., a polypeptide represented by UniProt Accession number Q96H96 or a polypeptide comprising an amino acid sequence having at least 95% sequence identity thereto).
1115. The method of any one of embodiments 431 to 434, wherein the disease or condition is Myasthenic syndrome, congenital presynaptic and/or wherein the heterologous nucleic acid sequence encodes CHAT (e.g., a polypeptide represented by UniProt Accession number P28329 or a polypeptide comprising an amino acid sequence having at least 95% sequence identity thereto).
1116. The method of any one of embodiments 431 to 434, wherein the disease or condition is Myoclonus and/or wherein the heterologous nucleic acid sequence encodes NOL3 (e.g., a polypeptide represented by UniProt Accession number O60936 or a polypeptide comprising an amino acid sequence having at least 95% sequence identity thereto).
1117. The method of any one of embodiments 431 to 434, wherein the disease or condition is Myoclonic epilepsy (FAME2) and/or wherein the heterologous nucleic acid sequence encodes STARD7 (e.g., a polypeptide represented by UniProt Accession number Q9NQZ5 or a polypeptide comprising an amino acid sequence having at least 95% sequence identity thereto).
1118. The method of any one of embodiments 431 to 434, wherein the disease or condition is Narcolepsy and/or wherein the heterologous nucleic acid sequence encodes HCRT, OX (e.g., a polypeptide represented by UniProt Accession number O43612 or a polypeptide comprising an amino acid sequence having at least 95% sequence identity thereto).
1119. The method of any one of embodiments 431 to 434, wherein the disease or condition is Narcolepsy and/or wherein the heterologous nucleic acid sequence encodes MOG (e.g., a polypeptide represented by UniProt Accession number Q16653 or a polypeptide comprising an amino acid sequence having at least 95% sequence identity thereto).
1120. The method of any one of embodiments 431 to 434, wherein the disease or condition is Neuroacanthocytosis (McLeod syndrome) and/or wherein the heterologous nucleic acid sequence encodes XK (e.g., a polypeptide represented by UniProt Accession number P51811 or a polypeptide comprising an amino acid sequence having at least 95% sequence identity thereto).
1121. The method of any one of embodiments 431 to 434, wherein the disease or condition is Neurodevelopmental disorder with cerebral atrophy and facial dysmorphism (NEDCAFD) and/or wherein the heterologous nucleic acid sequence encodes TTC5 (e.g., a polypeptide represented by UniProt Accession number Q8N0Z6 or a polypeptide comprising an amino acid sequence having at least 95% sequence identity thereto).
1122. The method of any one of embodiments 431 to 434, wherein the disease or condition is Neurodevelopmental disorder with infantile epileptic spasms and/or wherein the heterologous nucleic acid sequence encodes NCDN (e.g., a polypeptide represented by UniProt Accession number Q9UBB6 or a polypeptide comprising an amino acid sequence having at least 95% sequence identity thereto).
1123. The method of any one of embodiments 431 to 434, wherein the disease or condition is Neurofibromatosis and/or wherein the heterologous nucleic acid sequence encodes NF1 (e.g., a polypeptide represented by UniProt Accession number P21359 or a polypeptide comprising an amino acid sequence having at least 95% sequence identity thereto).
1124. The method of any one of embodiments 431 to 434, wherein the disease or condition is Neuromyotonia and/or wherein the heterologous nucleic acid sequence encodes HINT1 (e.g., a polypeptide represented by UniProt Accession number P49773 or a polypeptide comprising an amino acid sequence having at least 95% sequence identity thereto).
1125. The method of any one of embodiments 431 to 434, wherein the disease or condition is Neuronal Ceroid Lipofuscinosis and/or wherein the heterologous nucleic acid sequence encodes PPT1 (e.g., a polypeptide represented by UniProt Accession number P50897 or a polypeptide comprising an amino acid sequence having at least 95% sequence identity thereto).
1126. The method of any one of embodiments 431 to 434, wherein the disease or condition is Neuronal Ceroid Lipofuscinosis and/or wherein the heterologous nucleic acid sequence encodes TPP1 (e.g., a polypeptide represented by UniProt Accession number O14773 or a polypeptide comprising an amino acid sequence having at least 95% sequence identity thereto).
1127. The method of any one of embodiments 431 to 434, wherein the disease or condition is Neuronal Ceroid Lipofuscinosis and/or wherein the heterologous nucleic acid sequence encodes CLN5 (e.g., a polypeptide represented by UniProt Accession number O75503 or a polypeptide comprising an amino acid sequence having at least 95% sequence identity thereto).
1128. The method of any one of embodiments 431 to 434, wherein the disease or condition is Neuronal Ceroid Lipofuscinosis and/or wherein the heterologous nucleic acid sequence encodes CLN3 (e.g., a polypeptide represented by UniProt Accession number Q13286 or a polypeptide comprising an amino acid sequence having at least 95% sequence identity thereto).
1129. The method of any one of embodiments 431 to 434, wherein the disease or condition is Neuronal Ceroid Lipofuscinosis and/or wherein the heterologous nucleic acid sequence encodes CLN6 (e.g., a polypeptide represented by UniProt Accession number Q9NWW5 or a polypeptide comprising an amino acid sequence having at least 95% sequence identity thereto).
1130. The method of any one of embodiments 431 to 434, wherein the disease or condition is Neuronal Ceroid Lipofuscinosis and/or wherein the heterologous nucleic acid sequence encodes CLN8 (e.g., a polypeptide represented by UniProt Accession number Q9UBY8 or a polypeptide comprising an amino acid sequence having at least 95% sequence identity thereto).
1131. The method of any one of embodiments 431 to 434, wherein the disease or condition is Neuronal Ceroid Lipofuscinosis and/or wherein the heterologous nucleic acid sequence encodes DNAJC5 (e.g., a polypeptide represented by UniProt Accession number Q9H3Z4 or a polypeptide comprising an amino acid sequence having at least 95% sequence identity thereto).
1132. The method of any one of embodiments 431 to 434, wherein the disease or condition is Neuronal Ceroid Lipofuscinosis and/or wherein the heterologous nucleic acid sequence encodes MFSD8 (e.g., a polypeptide represented by UniProt Accession number Q8NHS3 or a polypeptide comprising an amino acid sequence having at least 95% sequence identity thereto).
1133. The method of any one of embodiments 431 to 434, wherein the disease or condition is Neuronal Ceroid Lipofuscinosis and/or wherein the heterologous nucleic acid sequence encodes CTSD (e.g., a polypeptide represented by UniProt Accession number P07339 or a polypeptide comprising an amino acid sequence having at least 95% sequence identity thereto).
1134. The method of any one of embodiments 431 to 434, wherein the disease or condition is Neuropathy, ataxia and retinitis pigmentosa (NARP) and/or wherein the heterologous nucleic acid sequence encodes MTATP6 (e.g., a polypeptide represented by UniProt Accession number P00846 or a polypeptide comprising an amino acid sequence having at least 95% sequence identity thereto).
1135. The method of any one of embodiments 431 to 434, wherein the disease or condition is Neuropathy, hereditary sensory and autonomic, type II and/or wherein the heterologous nucleic acid sequence encodes WNK1 (e.g., a polypeptide represented by UniProt Accession number Q9H4A3 or a polypeptide comprising an amino acid sequence having at least 95% sequence identity thereto).
1136. The method of any one of embodiments 431 to 434, wherein the disease or condition is Neuropathy, hypomyelinating congenital 1 and/or wherein the heterologous nucleic acid sequence encodes EGR2 (e.g., a polypeptide represented by UniProt Accession number P11161 or a polypeptide comprising an amino acid sequence having at least 95% sequence identity thereto).
1137. The method of any one of embodiments 431 to 434, wherein the disease or condition is Niemann-Pick disease and/or wherein the heterologous nucleic acid sequence encodes Sphingomyelin phosphodiesterase 1 (SMPD1) (e.g., a polypeptide represented by UniProt Accession number P17405 or a polypeptide comprising an amino acid sequence having at least 95% sequence identity thereto).
1138. The method of any one of embodiments 431 to 434, wherein the disease or condition is Niemann-Pick disease and/or wherein the heterologous nucleic acid sequence encodes NPC intracellular cholesterol transporter 1 (NPC1) (e.g., a polypeptide represented by UniProt Accession number O15118 or a polypeptide comprising an amino acid sequence having at least 95% sequence identity thereto).
1139. The method of any one of embodiments 431 to 434, wherein the disease or condition is Ohtahara Syndrome (Developmental and epileptic encephalopathy 1) and/or wherein the heterologous nucleic acid sequence encodes ARX (e.g., a polypeptide represented by UniProt Accession number Q96QS3 or a polypeptide comprising an amino acid sequence having at least 95% sequence identity thereto).
1140. The method of any one of embodiments 431 to 434, wherein the disease or condition is Omithine Transcarbamylase deficiency and/or wherein the heterologous nucleic acid sequence encodes OTC (e.g., a polypeptide represented by UniProt Accession number P00480 or a polypeptide comprising an amino acid sequence having at least 95% sequence identity thereto).
1141. The method of any one of embodiments 431 to 434, wherein the disease or condition is Orthostatic intolerance and/or wherein the heterologous nucleic acid sequence encodes SLC6A2 (e.g., a polypeptide represented by UniProt Accession number P23975 or a polypeptide comprising an amino acid sequence having at least 95% sequence identity thereto).
1142. The method of any one of embodiments 431 to 434, wherein the disease or condition is Parkinson's disease and/or wherein the heterologous nucleic acid sequence encodes Glucocerebrosidase (GBA1) (e.g., a polypeptide represented by UniProt Accession number P04062 or a polypeptide comprising an amino acid sequence having at least 95% sequence identity thereto).
1143. The method of any one of embodiments 431 to 434, wherein the disease or condition is Parkinson's disease and/or wherein the heterologous nucleic acid sequence encodes Dopamine decarboxylase (DDC) (e.g., a polypeptide represented by UniProt Accession number P20711 or a polypeptide comprising an amino acid sequence having at least 95% sequence identity thereto).
1144. The method of any one of embodiments 431 to 434, wherein the disease or condition is Parkinson's disease and/or wherein the heterologous nucleic acid sequence encodes Neurturin (e.g., a polypeptide represented by UniProt Accession number Q99748 or a polypeptide comprising an amino acid sequence having at least 95% sequence identity thereto).
1145. The method of any one of embodiments 431 to 434, wherein the disease or condition is Parkinson's disease and/or wherein the heterologous nucleic acid sequence encodes Glial derived growth factor (GDGF) (e.g., a polypeptide represented by UniProt Accession number P39905 or a polypeptide comprising an amino acid sequence having at least 95% sequence identity thereto).
1146. The method of any one of embodiments 431 to 434, wherein the disease or condition is Parkinson's disease and/or wherein the heterologous nucleic acid sequence encodes Tyrosine hydroxylase (TH), tyrosine 3-monooxygenase (e.g., a polypeptide represented by UniProt Accession number P07101 or a polypeptide comprising an amino acid sequence having at least 95% sequence identity thereto).
1147. The method of any one of embodiments 431 to 434, wherein the disease or condition is Parkinson's disease and/or wherein the heterologous nucleic acid sequence encodes Glutamic acid decarboxylase (GAD) (e.g., a polypeptide represented by UniProt Accession number Q99259 or a polypeptide comprising an amino acid sequence having at least 95% sequence identity thereto).
1148. The method of any one of embodiments 431 to 434, wherein the disease or condition is Parkinson's disease and/or wherein the heterologous nucleic acid sequence encodes Fibroblast growth factor 2 (FGF2) (e.g., a polypeptide represented by UniProt Accession number P09038 or a polypeptide comprising an amino acid sequence having at least 95% sequence identity thereto).
1149. The method of any one of embodiments 431 to 434, wherein the disease or condition is Parkinson's disease and/or wherein the heterologous nucleic acid sequence encodes Brain-derived neurotrophic factor (BDNF) (e.g., a polypeptide represented by UniProt Accession number P23560 or a polypeptide comprising an amino acid sequence having at least 95% sequence identity thereto).
1150. The method of any one of embodiments 431 to 434, wherein the disease or condition is Paroxysmal Choreoathetosis and/or wherein the heterologous nucleic acid sequence encodes PNKD (e.g., a polypeptide represented by UniProt Accession number Q8N490 or a polypeptide comprising an amino acid sequence having at least 95% sequence identity thereto).
1151. The method of any one of embodiments 431 to 434, wherein the disease or condition is Pelizaeus-Merzbacher Disease and/or wherein the heterologous nucleic acid sequence encodes PLP1 (e.g., a polypeptide represented by UniProt Accession number P60201 or a polypeptide comprising an amino acid sequence having at least 95% sequence identity thereto).
1152. The method of any one of embodiments 431 to 434, wherein the disease or condition is Pena-Shokeir Type II Syndrome and/or wherein the heterologous nucleic acid sequence encodes ERCC6 (e.g., a polypeptide represented by UniProt Accession number Q03468 or a polypeptide comprising an amino acid sequence having at least 95% sequence identity thereto).
1153. The method of any one of embodiments 431 to 434, wherein the disease or condition is Periodic Paralyses and/or wherein the heterologous nucleic acid sequence encodes SCN4A (e.g., a polypeptide represented by UniProt Accession number P35499 or a polypeptide comprising an amino acid sequence having at least 95% sequence identity thereto).
1154. The method of any one of embodiments 431 to 434, wherein the disease or condition is Phelan-McDermid syndrome and/or wherein the heterologous nucleic acid sequence encodes SH3 and multiple ankyrin repeat domains protein 3 (SHANK3) (e.g., a polypeptide represented by UniProt Accession number Q9BYB0 or a polypeptide comprising an amino acid sequence having at least 95% sequence identity thereto).
1155. The method of any one of embodiments 431 to 434, wherein the disease or condition is Phytanic Acid Storage Disease (peroxisome biogenesis disorder 1B) and/or wherein the heterologous nucleic acid sequence encodes PEX1 (e.g., a polypeptide represented by UniProt Accession number 043933 or a polypeptide comprising an amino acid sequence having at least 95% sequence identity thereto).
1156. The method of any one of embodiments 431 to 434, wherein the disease or condition is Pick disease and/or wherein the heterologous nucleic acid sequence encodes PSEN1 (e.g., a polypeptide represented by UniProt Accession number A0A024R6A3 or a polypeptide comprising an amino acid sequence having at least 95% sequence identity thereto).
1157. The method of any one of embodiments 431 to 434, wherein the disease or condition is Pick disease and/or wherein the heterologous nucleic acid sequence encodes MAPT (tau) (e.g., a polypeptide represented by UniProt Accession number P10636 or a polypeptide comprising an amino acid sequence having at least 95% sequence identity thereto).
1158. The method of any one of embodiments 431 to 434, wherein the disease or condition is Porencephaly type 1 and/or wherein the heterologous nucleic acid sequence encodes COL4A1 (e.g., a polypeptide represented by UniProt Accession number P02462 or a polypeptide comprising an amino acid sequence having at least 95% sequence identity thereto).
1159. The method of any one of embodiments 431 to 434, wherein the disease or condition is Primary Lateral Sclerosis, juvenile and/or wherein the heterologous nucleic acid sequence encodes ALS2 (e.g., a polypeptide represented by UniProt Accession number Q96Q42 or a polypeptide comprising an amino acid sequence having at least 95% sequence identity thereto).
1160. The method of any one of embodiments 431 to 434, wherein the disease or condition is Primary Progressive Aphasia and/or wherein the heterologous nucleic acid sequence encodes GRN (e.g., a polypeptide represented by UniProt Accession number P28799 or a polypeptide comprising an amino acid sequence having at least 95% sequence identity thereto).
1161. The method of any one of embodiments 431 to 434, wherein the disease or condition is Progressive external ophtalmoplegia and/or wherein the heterologous nucleic acid sequence encodes POLG (e.g., a polypeptide represented by UniProt Accession number P54098 or a polypeptide comprising an amino acid sequence having at least 95% sequence identity thereto).
1162. The method of any one of embodiments 431 to 434, wherein the disease or condition is Progressive external ophtalmoplegia and/or wherein the heterologous nucleic acid sequence encodes POLG2 (e.g., a polypeptide represented by UniProt Accession number Q9UHN1 or a polypeptide comprising an amino acid sequence having at least 95% sequence identity thereto).
1163. The method of any one of embodiments 431 to 434, wherein the disease or condition is Progressive external ophtalmoplegia and/or wherein the heterologous nucleic acid sequence encodes SLC25A4 (e.g., a polypeptide represented by UniProt Accession number P12235 or a polypeptide comprising an amino acid sequence having at least 95% sequence identity thereto).
1164. The method of any one of embodiments 431 to 434, wherein the disease or condition is Progressive external ophtalmoplegia and/or wherein the heterologous nucleic acid sequence encodes TWNK (e.g., a polypeptide represented by UniProt Accession number Q96RR1 or a polypeptide comprising an amino acid sequence having at least 95% sequence identity thereto).
1165. The method of any one of embodiments 431 to 434, wherein the disease or condition is Progressive bulbar palsy and/or wherein the heterologous nucleic acid sequence encodes SLC52A3 (e.g., a polypeptide represented by UniProt Accession number Q9NQ40 or a polypeptide comprising an amino acid sequence having at least 95% sequence identity thereto).
1166. The method of any one of embodiments 431 to 434, wherein the disease or condition is Progressive supranuclear palsy and/or wherein the heterologous nucleic acid sequence encodes Microtubule-associated protein tau (MAPT), Tau (e.g., a polypeptide represented by UniProt Accession number P10636 or a polypeptide comprising an amino acid sequence having at least 95% sequence identity thereto).
1167. The method of any one of embodiments 431 to 434, wherein the disease or condition is Pseudo-Torch syndrome and/or wherein the heterologous nucleic acid sequence encodes OCLN (e.g., a polypeptide represented by UniProt Accession number Q16625 or a polypeptide comprising an amino acid sequence having at least 95% sequence identity thereto).
1168. The method of any one of embodiments 431 to 434, wherein the disease or condition is Pseudo-Torch syndrome and/or wherein the heterologous nucleic acid sequence encodes STAT2 (e.g., a polypeptide represented by UniProt Accession number P52630 or a polypeptide comprising an amino acid sequence having at least 95% sequence identity thereto).
1169. The method of any one of embodiments 431 to 434, wherein the disease or condition is Pseudo-Torch syndrome and/or wherein the heterologous nucleic acid sequence encodes USP18 (e.g., a polypeptide represented by UniProt Accession number Q9UMW8 or a polypeptide comprising an amino acid sequence having at least 95% sequence identity thereto).
1170. The method of any one of embodiments 431 to 434, wherein the disease or condition is Refsum Disease and/or wherein the heterologous nucleic acid sequence encodes PHYH (e.g., a polypeptide represented by UniProt Accession number O14832 or a polypeptide comprising an amino acid sequence having at least 95% sequence identity thereto).
1171. The method of any one of embodiments 431 to 434, wherein the disease or condition is Retinitis Pigmentosa 38 (rod-cone dystrophy) and/or wherein the heterologous nucleic acid sequence encodes Tyrosine-protein kinase Mer (MERTK) (e.g., a polypeptide represented by UniProt Accession number Q12866 or a polypeptide comprising an amino acid sequence having at least 95% sequence identity thereto).
1172. The method of any one of embodiments 431 to 434, wherein the disease or condition is Retinitis Pigmentosa 40 and/or wherein the heterologous nucleic acid sequence encodes PDE6B (e.g., a polypeptide represented by UniProt Accession number P35913 or a polypeptide comprising an amino acid sequence having at least 95% sequence identity thereto).
1173. The method of any one of embodiments 431 to 434, wherein the disease or condition is Rett syndrome and/or wherein the heterologous nucleic acid sequence encodes Methyl-CpG-binding protein 2 (MECP2) (e.g., a polypeptide represented by UniProt Accession number P51608 or a polypeptide comprising an amino acid sequence having at least 95% sequence identity thereto).
1174. The method of any one of embodiments 431 to 434, wherein the disease or condition is Sandhoff disease and/or wherein the heterologous nucleic acid sequence encodes Beta-hexosaminidase subunit alpha (HEXA) (e.g., a polypeptide represented by UniProt Accession number P06865 or a polypeptide comprising an amino acid sequence having at least 95% sequence identity thereto).
1175. The method of any one of embodiments 431 to 434, wherein the disease or condition is Sandhoff disease and/or wherein the heterologous nucleic acid sequence encodes Beta-hexosaminidase subunit beta (HEXB) (e.g., a polypeptide represented by UniProt Accession number P07686 or a polypeptide comprising an amino acid sequence having at least 95% sequence identity thereto).
1176. The method of any one of embodiments 431 to 434, wherein the disease or condition is Schizencephaly and/or wherein the heterologous nucleic acid sequence encodes SIX3 (e.g., a polypeptide represented by UniProt Accession number O95343 or a polypeptide comprising an amino acid sequence having at least 95% sequence identity thereto).
1177. The method of any one of embodiments 431 to 434, wherein the disease or condition is Schizencephaly and/or wherein the heterologous nucleic acid sequence encodes EMX2 (e.g., a polypeptide represented by UniProt Accession number Q04743 or a polypeptide comprising an amino acid sequence having at least 95% sequence identity thereto).
1178. The method of any one of embodiments 431 to 434, wherein the disease or condition is Schizencephaly and/or wherein the heterologous nucleic acid sequence encodes SHH (e.g., a polypeptide represented by UniProt Accession number Q15465 or a polypeptide comprising an amino acid sequence having at least 95% sequence identity thereto).
1179. The method of any one of embodiments 431 to 434, wherein the disease or condition is Seitelberger Disease and/or wherein the heterologous nucleic acid sequence encodes PLA2G6 (e.g., a polypeptide represented by UniProt Accession number O60733 or a polypeptide comprising an amino acid sequence having at least 95% sequence identity thereto).
1180. The method of any one of embodiments 431 to 434, wherein the disease or condition is Septo-optic dysplasia (De Morsier syndrome) and/or wherein the heterologous nucleic acid sequence encodes HESX1 (e.g., a polypeptide represented by UniProt Accession number Q9UBX0 or a polypeptide comprising an amino acid sequence having at least 95% sequence identity thereto).
1181. The method of any one of embodiments 431 to 434, wherein the disease or condition is Snijders Blok-Fisher syndrome and/or wherein the heterologous nucleic acid sequence encodes POU3F3 (e.g., a polypeptide represented by UniProt Accession number P20264 or a polypeptide comprising an amino acid sequence having at least 95% sequence identity thereto).
1182. The method of any one of embodiments 431 to 434, wherein the disease or condition is Spastic Paraplegias and/or wherein the heterologous nucleic acid sequence encodes SPG11 (e.g., a polypeptide represented by UniProt Accession number Q96J17 or a polypeptide comprising an amino acid sequence having at least 95% sequence identity thereto).
1183. The method of any one of embodiments 431 to 434, wherein the disease or condition is Spastic Paraplegias and/or wherein the heterologous nucleic acid sequence encodes SPAST (e.g., a polypeptide represented by UniProt Accession number Q9UBP0 or a polypeptide comprising an amino acid sequence having at least 95% sequence identity thereto).
1184. The method of any one of embodiments 431 to 434, wherein the disease or condition is Spastic Paraplegias and/or wherein the heterologous nucleic acid sequence encodes KIF5A (e.g., a polypeptide represented by UniProt Accession number Q12840 or a polypeptide comprising an amino acid sequence having at least 95% sequence identity thereto).
1185. The method of any one of embodiments 431 to 434, wherein the disease or condition is Spastic Paraplegias and/or wherein the heterologous nucleic acid sequence encodes NIPA1 (e.g., a polypeptide represented by UniProt Accession number Q7RTP0 or a polypeptide comprising an amino acid sequence having at least 95% sequence identity thereto).
1186. The method of any one of embodiments 431 to 434, wherein the disease or condition is Spastic Paraplegias and/or wherein the heterologous nucleic acid sequence encodes CYP7B1 (e.g., a polypeptide represented by UniProt Accession number O75881 or a polypeptide comprising an amino acid sequence having at least 95% sequence identity thereto).
1187. The method of any one of embodiments 431 to 434, wherein the disease or condition is Spastic Paraplegias and/or wherein the heterologous nucleic acid sequence encodes ATL1 (e.g., a polypeptide represented by UniProt Accession number Q8WXF7 or a polypeptide comprising an amino acid sequence having at least 95% sequence identity thereto).
1188. The method of any one of embodiments 431 to 434, wherein the disease or condition is Spinal Muscular Atrophy (Kugelberg-Welander Disease) and/or wherein the heterologous nucleic acid sequence encodes Survival motor neuron protein (SMN), SMN1 (e.g., a polypeptide represented by UniProt Accession number Q16637 or a polypeptide comprising an amino acid sequence having at least 95% sequence identity thereto).
1189. The method of any one of embodiments 431 to 434, wherein the disease or condition is Spinocerebellar ataxia and/or wherein the heterologous nucleic acid sequence encodes Ataxin-1 (ATXN1), SCA1 (e.g., a polypeptide represented by UniProt Accession number P54253 or a polypeptide comprising an amino acid sequence having at least 95% sequence identity thereto).
1190. The method of any one of embodiments 431 to 434, wherein the disease or condition is Spinocerebellar ataxia and/or wherein the heterologous nucleic acid sequence encodes Ataxin-2 (ATXN2), SCA2 (e.g., a polypeptide represented by UniProt Accession number Q99700 or a polypeptide comprising an amino acid sequence having at least 95% sequence identity thereto).
1191. The method of any one of embodiments 431 to 434, wherein the disease or condition is Spinocerebellar ataxia and/or wherein the heterologous nucleic acid sequence encodes Ataxin-3 (ATXN3), SCA3 (e.g., a polypeptide represented by UniProt Accession number P54252 or a polypeptide comprising an amino acid sequence having at least 95% sequence identity thereto).
1192. The method of any one of embodiments 431 to 434, wherein the disease or condition is Spinocerebellar ataxia and/or wherein the heterologous nucleic acid sequence encodes ZFHX3 (e.g., a polypeptide represented by UniProt Accession number Q15911 or a polypeptide comprising an amino acid sequence having at least 95% sequence identity thereto).
1193. The method of any one of embodiments 431 to 434, wherein the disease or condition is Spinocerebellar ataxia and/or wherein the heterologous nucleic acid sequence encodes CACNA1A (e.g., a polypeptide represented by UniProt Accession number O00555 or a polypeptide comprising an amino acid sequence having at least 95% sequence identity thereto).
1194. The method of any one of embodiments 431 to 434, wherein the disease or condition is Spinocerebellar ataxia and/or wherein the heterologous nucleic acid sequence encodes ATXN7, SCA7 (e.g., a polypeptide represented by UniProt Accession number O15265 or a polypeptide comprising an amino acid sequence having at least 95% sequence identity thereto).
1195. The method of any one of embodiments 431 to 434, wherein the disease or condition is Spinocerebellar ataxia and/or wherein the heterologous nucleic acid sequence encodes TMEM240 (e.g., a polypeptide represented by UniProt Accession number Q5SV17 or a polypeptide comprising an amino acid sequence having at least 95% sequence identity thereto).
1196. The method of any one of embodiments 431 to 434, wherein the disease or condition is Sporadic Inclusion Body Myositis and/or wherein the heterologous nucleic acid sequence encodes Follistatin (FST) (e.g., a polypeptide represented by UniProt Accession number P19883 or a polypeptide comprising an amino acid sequence having at least 95% sequence identity thereto).
1197. The method of any one of embodiments 431 to 434, wherein the disease or condition is Steele-Richardson-Olszewski syndrome (Parkinson-dementia syndrome) and/or wherein the heterologous nucleic acid sequence encodes MAPT(Tau) (e.g., a polypeptide represented by UniProt Accession number P10636 or a polypeptide comprising an amino acid sequence having at least 95% sequence identity thereto).
1198. The method of any one of embodiments 431 to 434, wherein the disease or condition is Stiff-Person Syndrome, congenital and/or wherein the heterologous nucleic acid sequence encodes GLRA1 (e.g., a polypeptide represented by UniProt Accession number P23415 or a polypeptide comprising an amino acid sequence having at least 95% sequence identity thereto).
1199. The method of any one of embodiments 431 to 434, wherein the disease or condition is Stiff-Person Syndrome, congenital and/or wherein the heterologous nucleic acid sequence encodes GLRB (e.g., a polypeptide represented by UniProt Accession number P48167 or a polypeptide comprising an amino acid sequence having at least 95% sequence identity thereto).
1200. The method of any one of embodiments 431 to 434, wherein the disease or condition is Striatonigral degeneration and/or wherein the heterologous nucleic acid sequence encodes NUP62 (e.g., a polypeptide represented by UniProt Accession number P37198 or a polypeptide comprising an amino acid sequence having at least 95% sequence identity thereto).
1201. The method of any one of embodiments 431 to 434, wherein the disease or condition is Striatonigral degeneration and/or wherein the heterologous nucleic acid sequence encodes PDE8B (e.g., a polypeptide represented by UniProt Accession number O95263 or a polypeptide comprising an amino acid sequence having at least 95% sequence identity thereto).
1202. The method of any one of embodiments 431 to 434, wherein the disease or condition is Striatonigral degeneration and/or wherein the heterologous nucleic acid sequence encodes MTATP6 (e.g., a polypeptide represented by UniProt Accession number P00846 or a polypeptide comprising an amino acid sequence having at least 95% sequence identity thereto).
1203. The method of any one of embodiments 431 to 434, wherein the disease or condition is Striatonigral degeneration and/or wherein the heterologous nucleic acid sequence encodes VAC14 (e.g., a polypeptide represented by UniProt Accession number Q08AM6 or a polypeptide comprising an amino acid sequence having at least 95% sequence identity thereto).
1204. The method of any one of embodiments 431 to 434, wherein the disease or condition is Sturge-Weber Syndrome and/or wherein the heterologous nucleic acid sequence encodes GNAQ (e.g., a polypeptide represented by UniProt Accession number P50148 or a polypeptide comprising an amino acid sequence having at least 95% sequence identity thereto).
1205. The method of any one of embodiments 431 to 434, wherein the disease or condition is Subcortical Vascular Encephalopathy (Cerebral Arteriopathy) and/or wherein the heterologous nucleic acid sequence encodes HTRA1 (e.g., a polypeptide represented by UniProt Accession number Q92743 or a polypeptide comprising an amino acid sequence having at least 95% sequence identity thereto).
1206. The method of any one of embodiments 431 to 434, wherein the disease or condition is Systemic Lupus Erythematosus and/or wherein the heterologous nucleic acid sequence encodes DNASE1L3 (e.g., a polypeptide represented by UniProt Accession number Q13609 or a polypeptide comprising an amino acid sequence having at least 95% sequence identity thereto).
1207. The method of any one of embodiments 431 to 434, wherein the disease or condition is Systemic Lupus Erythematosus and/or wherein the heterologous nucleic acid sequence encodes TLR7 (e.g., a polypeptide represented by UniProt Accession number Q9NYK1 or a polypeptide comprising an amino acid sequence having at least 95% sequence identity thereto).
1208. The method of any one of embodiments 431 to 434, wherein the disease or condition is Tardive Dyskinesia and/or wherein the heterologous nucleic acid sequence encodes CYP2D6 (e.g., a polypeptide represented by UniProt Accession number P10635 or a polypeptide comprising an amino acid sequence having at least 95% sequence identity thereto).
1209. The method of any one of embodiments 431 to 434, wherein the disease or condition is Tay-Sachs disease and/or wherein the heterologous nucleic acid sequence encodes Beta-hexosaminidase subunit alpha (HEXA) (e.g., a polypeptide represented by UniProt Accession number P06865 or a polypeptide comprising an amino acid sequence having at least 95% sequence identity thereto).
1210. The method of any one of embodiments 431 to 434, wherein the disease or condition is Tourette Syndrome and/or wherein the heterologous nucleic acid sequence encodes HDC (e.g., a polypeptide represented by UniProt Accession number P19113 or a polypeptide comprising an amino acid sequence having at least 95% sequence identity thereto).
1211. The method of any one of embodiments 431 to 434, wherein the disease or condition is Tourette Syndrome and/or wherein the heterologous nucleic acid sequence encodes SLITRK1 (e.g., a polypeptide represented by UniProt Accession number Q96PX8 or a polypeptide comprising an amino acid sequence having at least 95% sequence identity thereto).
1212. The method of any one of embodiments 431 to 434, wherein the disease or condition is Tremor, hereditary type 1 and/or wherein the heterologous nucleic acid sequence encodes DRD3 (e.g., a polypeptide represented by UniProt Accession number P35462 or a polypeptide comprising an amino acid sequence having at least 95% sequence identity thereto).
1213. The method of any one of embodiments 431 to 434, wherein the disease or condition is Troyer Syndrome and/or wherein the heterologous nucleic acid sequence encodes SPART (e.g., a polypeptide represented by UniProt Accession number Q8N0X7 or a polypeptide comprising an amino acid sequence having at least 95% sequence identity thereto).
1214. The method of any one of embodiments 431 to 434, wherein the disease or condition is Tuberous Sclerosis and/or wherein the heterologous nucleic acid sequence encodes TSC1 (e.g., a polypeptide represented by UniProt Accession number Q92574 or a polypeptide comprising an amino acid sequence having at least 95% sequence identity thereto).
1215. The method of any one of embodiments 431 to 434, wherein the disease or condition is Tuberous Sclerosis and/or wherein the heterologous nucleic acid sequence encodes TSC2 (e.g., a polypeptide represented by UniProt Accession number P49815 or a polypeptide comprising an amino acid sequence having at least 95% sequence identity thereto).
1216. The method of any one of embodiments 431 to 434, wherein the disease or condition is Tuberous Sclerosis and/or wherein the heterologous nucleic acid sequence encodes IFNG (e.g., a polypeptide represented by UniProt Accession number P01579 or a polypeptide comprising an amino acid sequence having at least 95% sequence identity thereto).
1217. The method of any one of embodiments 431 to 434, wherein the disease or condition is Von Hippel-Lindau Disease and/or wherein the heterologous nucleic acid sequence encodes VHL (e.g., a polypeptide represented by UniProt Accession number P40337 or a polypeptide comprising an amino acid sequence having at least 95% sequence identity thereto).
1218. The method of any one of embodiments 431 to 434, wherein the disease or condition is Von Hippel-Lindau Disease and/or wherein the heterologous nucleic acid sequence encodes CCND1 (e.g., a polypeptide represented by UniProt Accession number P24385 or a polypeptide comprising an amino acid sequence having at least 95% sequence identity thereto).
1219. The method of any one of embodiments 431 to 434, wherein the disease or condition is Von Recklinghausen Disease and/or wherein the heterologous nucleic acid sequence encodes NF1 (e.g., a polypeptide represented by UniProt Accession number P21359 or a polypeptide comprising an amino acid sequence having at least 95% sequence identity thereto).
1220. The method of any one of embodiments 431 to 434, wherein the disease or condition is Werdnig-Hoffman Disease and/or wherein the heterologous nucleic acid sequence encodes SMN1 (e.g., a polypeptide represented by UniProt Accession number Q16637 or a polypeptide comprising an amino acid sequence having at least 95% sequence identity thereto).
1221. The method of any one of embodiments 431 to 434, wherein the disease or condition is West Syndrome, X-linked and/or wherein the heterologous nucleic acid sequence encodes ARX (e.g., a polypeptide represented by UniProt Accession number Q96QS3 or a polypeptide comprising an amino acid sequence having at least 95% sequence identity thereto).
1222. The method of any one of embodiments 431 to 434, wherein the disease or condition is Wilson disease and/or wherein the heterologous nucleic acid sequence encodes ATP7B (e.g., a polypeptide represented by UniProt Accession number P35670 or a polypeptide comprising an amino acid sequence having at least 95% sequence identity thereto).
1223. The method of any one of embodiments 431 to 434, wherein the disease or condition is Wolman's disease (acid lipase disease) and/or wherein the heterologous nucleic acid sequence encodes LIPA (e.g., a polypeptide represented by UniProt Accession number P38571 or a polypeptide comprising an amino acid sequence having at least 95% sequence identity thereto).
1224. The method of any one of embodiments 431 to 434, wherein the disease or condition is X-linked adrenoleukodystrophy and/or wherein the heterologous nucleic acid sequence encodes ATP-binding cassette sub-family D member 1 (ABCD1) (e.g., a polypeptide represented by UniProt Accession number P33897 or a polypeptide comprising an amino acid sequence having at least 95% sequence identity thereto).
1225. The method of any one of embodiments 431 to 434, wherein the disease or condition is X-linked Retinoschisis and/or wherein the heterologous nucleic acid sequence encodes Retinoschisin (RS1) (e.g., a polypeptide represented by UniProt Accession number O15537 or a polypeptide comprising an amino acid sequence having at least 95% sequence identity thereto).
1226. The method of any one of embodiments 431 to 434, wherein the disease or condition is X-Linked Retinitis Pigmentosa and/or wherein the heterologous nucleic acid sequence encodes X-linked retinitis pigmentosa GTPase regulator (RPGR) (e.g., a polypeptide represented by UniProt Accession number Q92834 or a polypeptide comprising an amino acid sequence having at least 95% sequence identity thereto).
1227. The method of any one of embodiments 431 to 434, wherein the disease or condition is X-Linked Spinal and Bulbar Muscular Atrophy and/or wherein the heterologous nucleic acid sequence encodes UBA1 (e.g., a polypeptide represented by UniProt Accession number P22314 or a polypeptide comprising an amino acid sequence having at least 95% sequence identity thereto).
1228. The method of any one of embodiments 431 to 434, wherein the disease or condition is Advanced heart failure and/or wherein the heterologous nucleic acid sequence encodes SERCA2a, ATP2A2 (e.g., a polypeptide represented by UniProt Accession number P16615 or a polypeptide comprising an amino acid sequence having at least 95% sequence identity thereto).
1229. The method of any one of embodiments 431 to 434, wherein the disease or condition is Amyotrophic lateral sclerosis (ALS) (Lou Gehrig's disease) and/or wherein the heterologous nucleic acid sequence encodes Superoxide dismutase-1 (SOD1) (e.g., a polypeptide represented by UniProt Accession number P00441 or a polypeptide comprising an amino acid sequence having at least 95% sequence identity thereto).
1230. The method of any one of embodiments 431 to 434, wherein the disease or condition is Andersen-Tawil Syndrome and/or wherein the heterologous nucleic acid sequence encodes KCNJ2 (e.g., a polypeptide represented by UniProt Accession number P63252 or a polypeptide comprising an amino acid sequence having at least 95% sequence identity thereto).
1231. The method of any one of embodiments 431 to 434, wherein the disease or condition is Barth syndrome and/or wherein the heterologous nucleic acid sequence encodes TAFAZZIN (e.g., a polypeptide represented by UniProt Accession number Q16635 or a polypeptide comprising an amino acid sequence having at least 95% sequence identity thereto).
1232. The method of any one of embodiments 431 to 434, wherein the disease or condition is Becker Muscular Dystrophy (BMD) and/or wherein the heterologous nucleic acid sequence encodes DMD (e.g., a polypeptide represented by UniProt Accession number P11532 or a polypeptide comprising an amino acid sequence having at least 95% sequence identity thereto).
1233. The method of any one of embodiments 431 to 434, wherein the disease or condition is Becker Myotonia Congenita and/or wherein the heterologous nucleic acid sequence encodes CLCN1 (e.g., a polypeptide represented by UniProt Accession number P35523 or a polypeptide comprising an amino acid sequence having at least 95% sequence identity thereto).
1234. The method of any one of embodiments 431 to 434, wherein the disease or condition is Bethlem Myopathy and/or wherein the heterologous nucleic acid sequence encodes COL6A3 (e.g., a polypeptide represented by UniProt Accession number P12111 or a polypeptide comprising an amino acid sequence having at least 95% sequence identity thereto).
1235. The method of any one of embodiments 431 to 434, wherein the disease or condition is Bethlem Myopathy and/or wherein the heterologous nucleic acid sequence encodes COL6A2 (e.g., a polypeptide represented by UniProt Accession number P12110 or a polypeptide comprising an amino acid sequence having at least 95% sequence identity thereto).
1236. The method of any one of embodiments 431 to 434, wherein the disease or condition is Bethlem Myopathy and/or wherein the heterologous nucleic acid sequence encodes COL6A1 (e.g., a polypeptide represented by UniProt Accession number P12109 or a polypeptide comprising an amino acid sequence having at least 95% sequence identity thereto).
1237. The method of any one of embodiments 431 to 434, wherein the disease or condition is Bulbospinal Muscular Atrophy and/or wherein the heterologous nucleic acid sequence encodes AR (e.g., a polypeptide represented by UniProt Accession number P10275 or a polypeptide comprising an amino acid sequence having at least 95% sequence identity thereto).
1238. The method of any one of embodiments 431 to 434, wherein the disease or condition is Carnitine Deficiency, systemic primary and/or wherein the heterologous nucleic acid sequence encodes SLC22A5 (e.g., a polypeptide represented by UniProt Accession number O76082 or a polypeptide comprising an amino acid sequence having at least 95% sequence identity thereto).
1239. The method of any one of embodiments 431 to 434, wherein the disease or condition is Carnitine Palmityl Transferase Deficiency, type 1 and/or wherein the heterologous nucleic acid sequence encodes CPT1A (e.g., a polypeptide represented by UniProt Accession number P50416 or a polypeptide comprising an amino acid sequence having at least 95% sequence identity thereto).
1240. The method of any one of embodiments 431 to 434, wherein the disease or condition is Carnitine Palmityl Transferase Deficiency, type 2 and/or wherein the heterologous nucleic acid sequence encodes CPT2 (e.g., a polypeptide represented by UniProt Accession number P23786 or a polypeptide comprising an amino acid sequence having at least 95% sequence identity thereto).
1241. The method of any one of embodiments 431 to 434, wherein the disease or condition is Catecholaminergic polymorphic ventricular tachycardia 2 (CPVT2) and/or wherein the heterologous nucleic acid sequence encodes Calsequestrin-2 (CASQ2) (e.g., a polypeptide represented by UniProt Accession number 014958 or a polypeptide comprising an amino acid sequence having at least 95% sequence identity thereto).
1242. The method of any one of embodiments 431 to 434, wherein the disease or condition is Central Core Disease (congenital myopathy, type 1A) and/or wherein the heterologous nucleic acid sequence encodes RYR1 (e.g., a polypeptide represented by UniProt Accession number P21817 or a polypeptide comprising an amino acid sequence having at least 95% sequence identity thereto).
1243. The method of any one of embodiments 431 to 434, wherein the disease or condition is Centronuclear Myopathy type 1 and/or wherein the heterologous nucleic acid sequence encodes MTMR14 (e.g., a polypeptide represented by UniProt Accession number Q8NCE2 or a polypeptide comprising an amino acid sequence having at least 95% sequence identity thereto).
1244. The method of any one of embodiments 431 to 434, wherein the disease or condition is Centronuclear Myopathy type 2 and/or wherein the heterologous nucleic acid sequence encodes DNM2 (e.g., a polypeptide represented by UniProt Accession number O14717 or a polypeptide comprising an amino acid sequence having at least 95% sequence identity thereto).
1245. The method of any one of embodiments 431 to 434, wherein the disease or condition is Charcot-Marie-Tooth disease and/or wherein the heterologous nucleic acid sequence encodes PMP22 (e.g., a polypeptide represented by UniProt Accession number Q01453 or a polypeptide comprising an amino acid sequence having at least 95% sequence identity thereto).
1246. The method of any one of embodiments 431 to 434, wherein the disease or condition is Charcot-Marie-Tooth disease and/or wherein the heterologous nucleic acid sequence encodes MPZ (e.g., a polypeptide represented by UniProt Accession number P25189 or a polypeptide comprising an amino acid sequence having at least 95% sequence identity thereto).
1247. The method of any one of embodiments 431 to 434, wherein the disease or condition is Charcot-Marie-Tooth disease and/or wherein the heterologous nucleic acid sequence encodes DNM2 (e.g., a polypeptide represented by UniProt Accession number P50570 or a polypeptide comprising an amino acid sequence having at least 95% sequence identity thereto).
1248. The method of any one of embodiments 431 to 434, wherein the disease or condition is Charcot-Marie-Tooth disease and/or wherein the heterologous nucleic acid sequence encodes MFN2 (e.g., a polypeptide represented by UniProt Accession number O95140 or a polypeptide comprising an amino acid sequence having at least 95% sequence identity thereto).
1249. The method of any one of embodiments 431 to 434, wherein the disease or condition is Charcot-Marie-Tooth disease and/or wherein the heterologous nucleic acid sequence encodes KIF1B (e.g., a polypeptide represented by UniProt Accession number O60333 or a polypeptide comprising an amino acid sequence having at least 95% sequence identity thereto).
1250. The method of any one of embodiments 431 to 434, wherein the disease or condition is Charcot-Marie-Tooth disease and/or wherein the heterologous nucleic acid sequence encodes SBF2 (e.g., a polypeptide represented by UniProt Accession number Q86WG5 or a polypeptide comprising an amino acid sequence having at least 95% sequence identity thereto).
1251. The method of any one of embodiments 431 to 434, wherein the disease or condition is Charcot-Marie-Tooth disease and/or wherein the heterologous nucleic acid sequence encodes PNKP (e.g., a polypeptide represented by UniProt Accession number Q96T60 or a polypeptide comprising an amino acid sequence having at least 95% sequence identity thereto).
1252. The method of any one of embodiments 431 to 434, wherein the disease or condition is Charcot-Marie-Tooth disease and/or wherein the heterologous nucleic acid sequence encodes GDAP1 (e.g., a polypeptide represented by UniProt Accession number Q8TB36 or a polypeptide comprising an amino acid sequence having at least 95% sequence identity thereto).
1253. The method of any one of embodiments 431 to 434, wherein the disease or condition is Charcot-Marie-Tooth disease and/or wherein the heterologous nucleic acid sequence encodes LMNA (e.g., a polypeptide represented by UniProt Accession number P02545 or a polypeptide comprising an amino acid sequence having at least 95% sequence identity thereto).
1254. The method of any one of embodiments 431 to 434, wherein the disease or condition is Charcot-Marie-Tooth disease and/or wherein the heterologous nucleic acid sequence encodes FGD4 (e.g., a polypeptide represented by UniProt Accession number Q96M96 or a polypeptide comprising an amino acid sequence having at least 95% sequence identity thereto).
1255. The method of any one of embodiments 431 to 434, wherein the disease or condition is Charcot-Marie-Tooth disease and/or wherein the heterologous nucleic acid sequence encodes MTMR2 (e.g., a polypeptide represented by UniProt Accession number Q13614 or a polypeptide comprising an amino acid sequence having at least 95% sequence identity thereto).
1256. The method of any one of embodiments 431 to 434, wherein the disease or condition is Congenital Muscular Dystrophy (Ullrich disease) and/or wherein the heterologous nucleic acid sequence encodes COL6A3 (e.g., a polypeptide represented by UniProt Accession number P12111 or a polypeptide comprising an amino acid sequence having at least 95% sequence identity thereto).
1257. The method of any one of embodiments 431 to 434, wherein the disease or condition is Congenital Muscular Dystrophy (Ullrich disease) and/or wherein the heterologous nucleic acid sequence encodes COL6A2 (e.g., a polypeptide represented by UniProt Accession number P12110 or a polypeptide comprising an amino acid sequence having at least 95% sequence identity thereto).
1258. The method of any one of embodiments 431 to 434, wherein the disease or condition is Congenital Muscular Dystrophy (Ullrich disease) and/or wherein the heterologous nucleic acid sequence encodes COL6A1 (e.g., a polypeptide represented by UniProt Accession number P12109 or a polypeptide comprising an amino acid sequence having at least 95% sequence identity thereto).
1259. The method of any one of embodiments 431 to 434, wherein the disease or condition is Congenital Myasthenic Syndrome and/or wherein the heterologous nucleic acid sequence encodes COLQ (e.g., a polypeptide represented by UniProt Accession number Q9Y215 or a polypeptide comprising an amino acid sequence having at least 95% sequence identity thereto).
1260. The method of any one of embodiments 431 to 434, wherein the disease or condition is Congenital Myasthenic Syndrome and/or wherein the heterologous nucleic acid sequence encodes AGRN (e.g., a polypeptide represented by UniProt Accession number O00468 or a polypeptide comprising an amino acid sequence having at least 95% sequence identity thereto).
1261. The method of any one of embodiments 431 to 434, wherein the disease or condition is Congenital Myasthenic Syndrome and/or wherein the heterologous nucleic acid sequence encodes RAPSN (e.g., a polypeptide represented by UniProt Accession number Q13702 or a polypeptide comprising an amino acid sequence having at least 95% sequence identity thereto).
1262. The method of any one of embodiments 431 to 434, wherein the disease or condition is Congenital Myasthenic Syndrome and/or wherein the heterologous nucleic acid sequence encodes GFPT1 (e.g., a polypeptide represented by UniProt Accession number Q06210 or a polypeptide comprising an amino acid sequence having at least 95% sequence identity thereto).
1263. The method of any one of embodiments 431 to 434, wherein the disease or condition is Congenital Myasthenic Syndrome and/or wherein the heterologous nucleic acid sequence encodes SCN4A (e.g., a polypeptide represented by UniProt Accession number P35499 or a polypeptide comprising an amino acid sequence having at least 95% sequence identity thereto).
1264. The method of any one of embodiments 431 to 434, wherein the disease or condition is Congenital Myasthenic Syndrome and/or wherein the heterologous nucleic acid sequence encodes ALG2 (e.g., a polypeptide represented by UniProt Accession number Q9H553 or a polypeptide comprising an amino acid sequence having at least 95% sequence identity thereto).
1265. The method of any one of embodiments 431 to 434, wherein the disease or condition is Congenital Myasthenic Syndrome and/or wherein the heterologous nucleic acid sequence encodes ALG14 (e.g., a polypeptide represented by UniProt Accession number Q96F25 or a polypeptide comprising an amino acid sequence having at least 95% sequence identity thereto).
1266. The method of any one of embodiments 431 to 434, wherein the disease or condition is Congenital Myasthenic Syndrome and/or wherein the heterologous nucleic acid sequence encodes DPAGT1 (e.g., a polypeptide represented by UniProt Accession number Q9H3H5 or a polypeptide comprising an amino acid sequence having at least 95% sequence identity thereto).
1267. The method of any one of embodiments 431 to 434, wherein the disease or condition is Congenital Myasthenic Syndrome and/or wherein the heterologous nucleic acid sequence encodes CHRNE (e.g., a polypeptide represented by UniProt Accession number Q04844 or a polypeptide comprising an amino acid sequence having at least 95% sequence identity thereto).
1268. The method of any one of embodiments 431 to 434, wherein the disease or condition is Congenital Myasthenic Syndrome and/or wherein the heterologous nucleic acid sequence encodes CHRNA1 (e.g., a polypeptide represented by UniProt Accession number P02708 or a polypeptide comprising an amino acid sequence having at least 95% sequence identity thereto).
1269. The method of any one of embodiments 431 to 434, wherein the disease or condition is Congenital Myasthenic Syndrome and/or wherein the heterologous nucleic acid sequence encodes DOK7 (e.g., a polypeptide represented by UniProt Accession number Q18PE1 or a polypeptide comprising an amino acid sequence having at least 95% sequence identity thereto).
1270. The method of any one of embodiments 431 to 434, wherein the disease or condition is Congenital Myasthenic Syndrome and/or wherein the heterologous nucleic acid sequence encodes CHAT (e.g., a polypeptide represented by UniProt Accession number P28329 or a polypeptide comprising an amino acid sequence having at least 95% sequence identity thereto).
1271. The method of any one of embodiments 431 to 434, wherein the disease or condition is Congenital Myopathy and/or wherein the heterologous nucleic acid sequence encodes ACTA1 (e.g., a polypeptide represented by UniProt Accession number P68133 or a polypeptide comprising an amino acid sequence having at least 95% sequence identity thereto).
1272. The method of any one of embodiments 431 to 434, wherein the disease or condition is Congenital Myopathy and/or wherein the heterologous nucleic acid sequence encodes STAC3 (e.g., a polypeptide represented by UniProt Accession number Q96MF2 or a polypeptide comprising an amino acid sequence having at least 95% sequence identity thereto).
1273. The method of any one of embodiments 431 to 434, wherein the disease or condition is Congenital Myopathy and/or wherein the heterologous nucleic acid sequence encodes TPM3 (e.g., a polypeptide represented by UniProt Accession number P06753 or a polypeptide comprising an amino acid sequence having at least 95% sequence identity thereto).
1274. The method of any one of embodiments 431 to 434, wherein the disease or condition is Congenital Myotonic Dystrophy and/or wherein the heterologous nucleic acid sequence encodes DMPK (e.g., a polypeptide represented by UniProt Accession number Q09013 or a polypeptide comprising an amino acid sequence having at least 95% sequence identity thereto).
1275. The method of any one of embodiments 431 to 434, wherein the disease or condition is Cori Disease (Debrancher Enzyme Deficiency or Forbes Disease) and/or wherein the heterologous nucleic acid sequence encodes AGL (e.g., a polypeptide represented by UniProt Accession number P35573 or a polypeptide comprising an amino acid sequence having at least 95% sequence identity thereto).
1276. The method of any one of embodiments 431 to 434, wherein the disease or condition is Danon disease and/or wherein the heterologous nucleic acid sequence encodes LAMP2 (e.g., a polypeptide represented by UniProt Accession number P13473 or a polypeptide comprising an amino acid sequence having at least 95% sequence identity thereto).
1277. The method of any one of embodiments 431 to 434, wherein the disease or condition is Dejerine-Sottas Disease and/or wherein the heterologous nucleic acid sequence encodes MPZ (e.g., a polypeptide represented by UniProt Accession number P25189 or a polypeptide comprising an amino acid sequence having at least 95% sequence identity thereto).
1278. The method of any one of embodiments 431 to 434, wherein the disease or condition is Dejerine-Sottas Disease and/or wherein the heterologous nucleic acid sequence encodes EGR2 (e.g., a polypeptide represented by UniProt Accession number P11161 or a polypeptide comprising an amino acid sequence having at least 95% sequence identity thereto).
1279. The method of any one of embodiments 431 to 434, wherein the disease or condition is Dejerine-Sottas Disease and/or wherein the heterologous nucleic acid sequence encodes PMP22 (e.g., a polypeptide represented by UniProt Accession number Q01453 or a polypeptide comprising an amino acid sequence having at least 95% sequence identity thereto).
1280. The method of any one of embodiments 431 to 434, wherein the disease or condition is Dejerine-Sottas Disease and/or wherein the heterologous nucleic acid sequence encodes PRX (e.g., a polypeptide represented by UniProt Accession number Q9BXM0 or a polypeptide comprising an amino acid sequence having at least 95% sequence identity thereto).
1281. The method of any one of embodiments 431 to 434, wherein the disease or condition is Distal Muscular Dystrophy, Welander and/or wherein the heterologous nucleic acid sequence encodes TIA1 (e.g., a polypeptide represented by UniProt Accession number P31483 or a polypeptide comprising an amino acid sequence having at least 95% sequence identity thereto).
1282. The method of any one of embodiments 431 to 434, wherein the disease or condition is Distal Muscular Dystrophy, Miyoshi and/or wherein the heterologous nucleic acid sequence encodes DYSF (e.g., a polypeptide represented by UniProt Accession number O75923 or a polypeptide comprising an amino acid sequence having at least 95% sequence identity thereto).
1283. The method of any one of embodiments 431 to 434, wherein the disease or condition is Distal myopathy with anterior tibial onset and/or wherein the heterologous nucleic acid sequence encodes DYSF (e.g., a polypeptide represented by UniProt Accession number O75923 or a polypeptide comprising an amino acid sequence having at least 95% sequence identity thereto).
1284. The method of any one of embodiments 431 to 434, wherein the disease or condition is Duchenne Muscular Dystrophy and/or wherein the heterologous nucleic acid sequence encodes Dystrophin (DMD) (e.g., a polypeptide represented by UniProt Accession number P11532 or a polypeptide comprising an amino acid sequence having at least 95% sequence identity thereto).
1285. The method of any one of embodiments 431 to 434, wherein the disease or condition is Duchenne Muscular Dystrophy and/or wherein the heterologous nucleic acid sequence encodes GALGT2, B4GALNT2 (e.g., a polypeptide represented by UniProt Accession number Q8NHY0 or a polypeptide comprising an amino acid sequence having at least 95% sequence identity thereto).
1286. The method of any one of embodiments 431 to 434, wherein the disease or condition is Dysferlinopathy and/or wherein the heterologous nucleic acid sequence encodes Dysferlin (DYSF) (e.g., a polypeptide represented by UniProt Accession number O75923 or a polypeptide comprising an amino acid sequence having at least 95% sequence identity thereto).
1287. The method of any one of embodiments 431 to 434, wherein the disease or condition is Emery-Dreifuss Muscular Dystrophy and/or wherein the heterologous nucleic acid sequence encodes EMD (e.g., a polypeptide represented by UniProt Accession number P50402 or a polypeptide comprising an amino acid sequence having at least 95% sequence identity thereto).
1288. The method of any one of embodiments 431 to 434, wherein the disease or condition is Emery-Dreifuss Muscular Dystrophy and/or wherein the heterologous nucleic acid sequence encodes SYNE1 (e.g., a polypeptide represented by UniProt Accession number Q8NF91 or a polypeptide comprising an amino acid sequence having at least 95% sequence identity thereto).
1289. The method of any one of embodiments 431 to 434, wherein the disease or condition is Emery-Dreifuss Muscular Dystrophy and/or wherein the heterologous nucleic acid sequence encodes SYNE2 (e.g., a polypeptide represented by UniProt Accession number Q8WXH0 or a polypeptide comprising an amino acid sequence having at least 95% sequence identity thereto).
1290. The method of any one of embodiments 431 to 434, wherein the disease or condition is Emery-Dreifuss Muscular Dystrophy and/or wherein the heterologous nucleic acid sequence encodes TMEM43 (e.g., a polypeptide represented by UniProt Accession number Q9BTV4 or a polypeptide comprising an amino acid sequence having at least 95% sequence identity thereto).
1291. The method of any one of embodiments 431 to 434, wherein the disease or condition is Emery-Dreifuss Muscular Dystrophy and/or wherein the heterologous nucleic acid sequence encodes LMNA (e.g., a polypeptide represented by UniProt Accession number P02545 or a polypeptide comprising an amino acid sequence having at least 95% sequence identity thereto).
1292. The method of any one of embodiments 431 to 434, wherein the disease or condition is Eulenberg Disease (Paramyotonia Congenita) and/or wherein the heterologous nucleic acid sequence encodes SCN4A (e.g., a polypeptide represented by UniProt Accession number P35499 or a polypeptide comprising an amino acid sequence having at least 95% sequence identity thereto).
1293. The method of any one of embodiments 431 to 434, wherein the disease or condition is Facioscapulohumeral Muscular Dystrophy and/or wherein the heterologous nucleic acid sequence encodes SMCHD1 (e.g., a polypeptide represented by UniProt Accession number A6NHR9 or a polypeptide comprising an amino acid sequence having at least 95% sequence identity thereto).
1294. The method of any one of embodiments 431 to 434, wherein the disease or condition is Facioscapulohumeral Muscular Dystrophy and/or wherein the heterologous nucleic acid sequence encodes LRIF1 (e.g., a polypeptide represented by UniProt Accession number Q5T3J3 or a polypeptide comprising an amino acid sequence having at least 95% sequence identity thereto).
1295. The method of any one of embodiments 431 to 434, wherein the disease or condition is Friedreich's ataxia and/or wherein the heterologous nucleic acid sequence encodes Frataxin (FXN) (e.g., a polypeptide represented by UniProt Accession number Q16595 or a polypeptide comprising an amino acid sequence having at least 95% sequence identity thereto).
1296. The method of any one of embodiments 431 to 434, wherein the disease or condition is Fukuyama Congenital Muscular Dystrophy and/or wherein the heterologous nucleic acid sequence encodes FKTN (e.g., a polypeptide represented by UniProt Accession number O75072 or a polypeptide comprising an amino acid sequence having at least 95% sequence identity thereto).
1297. The method of any one of embodiments 431 to 434, wherein the disease or condition is Glycogen storage disease II (Pompe disease or Acid Maltase Deficiency) and/or wherein the heterologous nucleic acid sequence encodes GAA (e.g., a polypeptide represented by UniProt Accession number P10253 or a polypeptide comprising an amino acid sequence having at least 95% sequence identity thereto).
1298. The method of any one of embodiments 431 to 434, wherein the disease or condition is Glycogenosis Type 10 (Glycogen storage disease X) and/or wherein the heterologous nucleic acid sequence encodes PGAM2 (e.g., a polypeptide represented by UniProt Accession number P15259 or a polypeptide comprising an amino acid sequence having at least 95% sequence identity thereto).
1299. The method of any one of embodiments 431 to 434, wherein the disease or condition is Glycogenosis Type 11 (Glycogen storage disease XI) and/or wherein the heterologous nucleic acid sequence encodes LDHA (e.g., a polypeptide represented by UniProt Accession number P00338 or a polypeptide comprising an amino acid sequence having at least 95% sequence identity thereto).
1300. The method of any one of embodiments 431 to 434, wherein the disease or condition is Glycogenosis Type 2 (Glycogen storage disease II or Pompe Disease or Acid maltase deficiency) and/or wherein the heterologous nucleic acid sequence encodes GAA (e.g., a polypeptide represented by UniProt Accession number P10253 or a polypeptide comprising an amino acid sequence having at least 95% sequence identity thereto).
1301. The method of any one of embodiments 431 to 434, wherein the disease or condition is Glycogenosis Type 3 (Glycogen storage disease Ill) and/or wherein the heterologous nucleic acid sequence encodes AGL (e.g., a polypeptide represented by UniProt Accession number P35573 or a polypeptide comprising an amino acid sequence having at least 95% sequence identity thereto).
1302. The method of any one of embodiments 431 to 434, wherein the disease or condition is Glycogenosis Type 5 (Glycogen storage disease V or McArdle Disease or Myophosphorylase Deficiency) and/or wherein the heterologous nucleic acid sequence encodes PYGM (e.g., a polypeptide represented by UniProt Accession number P11217 or a polypeptide comprising an amino acid sequence having at least 95% sequence identity thereto).
1303. The method of any one of embodiments 431 to 434, wherein the disease or condition is Glycogenosis Type 7 (Glycogen storage disease VII or Phosphofructokinase Deficiency or Tarui Disease) and/or wherein the heterologous nucleic acid sequence encodes PFKM (e.g., a polypeptide represented by UniProt Accession number P08237 or a polypeptide comprising an amino acid sequence having at least 95% sequence identity thereto).
1304. The method of any one of embodiments 431 to 434, wherein the disease or condition is Glycogenosis Type 9 (Glycogen storage disease IX) and/or wherein the heterologous nucleic acid sequence encodes PHKB (e.g., a polypeptide represented by UniProt Accession number Q93100 or a polypeptide comprising an amino acid sequence having at least 95% sequence identity thereto).
1305. The method of any one of embodiments 431 to 434, wherein the disease or condition is Glycogenosis Type 9 (Glycogen storage disease IX) and/or wherein the heterologous nucleic acid sequence encodes PHKA2 (e.g., a polypeptide represented by UniProt Accession number P46019 or a polypeptide comprising an amino acid sequence having at least 95% sequence identity thereto).
1306. The method of any one of embodiments 431 to 434, wherein the disease or condition is Hereditary Inclusion-Body Myositis and/or wherein the heterologous nucleic acid sequence encodes GNE (e.g., a polypeptide represented by UniProt Accession number Q9Y223 or a polypeptide comprising an amino acid sequence having at least 95% sequence identity thereto).
1307. The method of any one of embodiments 431 to 434, wherein the disease or condition is Integrin-Deficient Congenital Muscular Dystrophy and/or wherein the heterologous nucleic acid sequence encodes ITGA7 (e.g., a polypeptide represented by UniProt Accession number Q13683 or a polypeptide comprising an amino acid sequence having at least 95% sequence identity thereto).
1308. The method of any one of embodiments 431 to 434, wherein the disease or condition is Kennedy Disease (Spinal-Bulbar Muscular Atrophy) and/or wherein the heterologous nucleic acid sequence encodes Androgen receptor (AR) (e.g., a polypeptide represented by UniProt Accession number P10275 or a polypeptide comprising an amino acid sequence having at least 95% sequence identity thereto).
1309. The method of any one of embodiments 431 to 434, wherein the disease or condition is Kugelberg-Welander Disease and/or wherein the heterologous nucleic acid sequence encodes SMN1 (e.g., a polypeptide represented by UniProt Accession number Q16637 or a polypeptide comprising an amino acid sequence having at least 95% sequence identity thereto).
1310. The method of any one of embodiments 431 to 434, wherein the disease or condition is Lactate dehydrogenase A deficiency and/or wherein the heterologous nucleic acid sequence encodes LDHA (e.g., a polypeptide represented by UniProt Accession number P00338 or a polypeptide comprising an amino acid sequence having at least 95% sequence identity thereto).
1311. The method of any one of embodiments 431 to 434, wherein the disease or condition is Lactate Dehydrogenase B Deficiency and/or wherein the heterologous nucleic acid sequence encodes LDHB (e.g., a polypeptide represented by UniProt Accession number P07195 or a polypeptide comprising an amino acid sequence having at least 95% sequence identity thereto).
1312. The method of any one of embodiments 431 to 434, wherein the disease or condition is Lambert-Eaton Myasthenic Syndrome and/or wherein the heterologous nucleic acid sequence encodes CACNB2 (e.g., a polypeptide represented by UniProt Accession number Q08289 or a polypeptide comprising an amino acid sequence having at least 95% sequence identity thereto).
1313. The method of any one of embodiments 431 to 434, wherein the disease or condition is Laing Distal Myopathy and/or wherein the heterologous nucleic acid sequence encodes MYH7 (e.g., a polypeptide represented by UniProt Accession number A7E2Y1 or a polypeptide comprising an amino acid sequence having at least 95% sequence identity thereto).
1314. The method of any one of embodiments 431 to 434, wherein the disease or condition is Limb Girdle Muscular Dystrophy Type 2C (LGMD-2C) and/or wherein the heterologous nucleic acid sequence encodes Gamma-sarcoglycan (e.g., a polypeptide represented by UniProt Accession number Q13326 or a polypeptide comprising an amino acid sequence having at least 95% sequence identity thereto).
1315. The method of any one of embodiments 431 to 434, wherein the disease or condition is Limb Girdle Muscular Dystrophy Type 2D (LGMD-2D) and/or wherein the heterologous nucleic acid sequence encodes Alpha-sarcoglycan (e.g., a polypeptide represented by UniProt Accession number Q16586 or a polypeptide comprising an amino acid sequence having at least 95% sequence identity thereto).
1316. The method of any one of embodiments 431 to 434, wherein the disease or condition is Limb Girdle Muscular Dystrophy Type2E (LGMD-2E) and/or wherein the heterologous nucleic acid sequence encodes Beta-sarcoglycan (e.g., a polypeptide represented by UniProt Accession number Q16585 or a polypeptide comprising an amino acid sequence having at least 95% sequence identity thereto).
1317. The method of any one of embodiments 431 to 434, wherein the disease or condition is Limb Girdle Muscular Dystrophy Type 2F (LGMD-2F) and/or wherein the heterologous nucleic acid sequence encodes Delta-sarcoglycan (e.g., a polypeptide represented by UniProt Accession number Q92629 or a polypeptide comprising an amino acid sequence having at least 95% sequence identity thereto).
1318. The method of any one of embodiments 431 to 434, wherein the disease or condition is Merosin-Deficient Congenital Muscular Dystrophy and/or wherein the heterologous nucleic acid sequence encodes LAMA2 (e.g., a polypeptide represented by UniProt Accession number P24043 or a polypeptide comprising an amino acid sequence having at least 95% sequence identity thereto).
1319. The method of any one of embodiments 431 to 434, wherein the disease or condition is Muscle-Eye-Brain Disease and/or wherein the heterologous nucleic acid sequence encodes POMGNT1 (e.g., a polypeptide represented by UniProt Accession number Q8WZA1 or a polypeptide comprising an amino acid sequence having at least 95% sequence identity thereto).
1320. The method of any one of embodiments 431 to 434, wherein the disease or condition is Mitochondrial Myopathy and/or wherein the heterologous nucleic acid sequence encodes CHCHD10 (e.g., a polypeptide represented by UniProt Accession number Q8WYQ3 or a polypeptide comprising an amino acid sequence having at least 95% sequence identity thereto).
1321. The method of any one of embodiments 431 to 434, wherein the disease or condition is Miyoshi myopathy and/or wherein the heterologous nucleic acid sequence encodes DYSF (e.g., a polypeptide represented by UniProt Accession number O75923 or a polypeptide comprising an amino acid sequence having at least 95% sequence identity thereto).
1322. The method of any one of embodiments 431 to 434, wherein the disease or condition is Myoadenylate Deaminase Deficiency and/or wherein the heterologous nucleic acid sequence encodes AMPD1 (e.g., a polypeptide represented by UniProt Accession number P23109 or a polypeptide comprising an amino acid sequence having at least 95% sequence identity thereto).
1323. The method of any one of embodiments 431 to 434, wherein the disease or condition is Myofibrillar Myopathy 1 and/or wherein the heterologous nucleic acid sequence encodes DES (e.g., a polypeptide represented by UniProt Accession number P17661 or a polypeptide comprising an amino acid sequence having at least 95% sequence identity thereto).
1324. The method of any one of embodiments 431 to 434, wherein the disease or condition is Myofibrillar Myopathy 2 and/or wherein the heterologous nucleic acid sequence encodes CRYAB (e.g., a polypeptide represented by UniProt Accession number P02511 or a polypeptide comprising an amino acid sequence having at least 95% sequence identity thereto).
1325. The method of any one of embodiments 431 to 434, wherein the disease or condition is Myofibrillar Myopathy 3 and/or wherein the heterologous nucleic acid sequence encodes MYOT (e.g., a polypeptide represented by UniProt Accession number Q9UBF9 or a polypeptide comprising an amino acid sequence having at least 95% sequence identity thereto).
1326. The method of any one of embodiments 431 to 434, wherein the disease or condition is Myofibrillar Myopathy 4 (ZASP related myopathy) and/or wherein the heterologous nucleic acid sequence encodes LDB3, ZASP (e.g., a polypeptide represented by UniProt Accession number O75112 or a polypeptide comprising an amino acid sequence having at least 95% sequence identity thereto).
1327. The method of any one of embodiments 431 to 434, wherein the disease or condition is Myofibrillar Myopathy 5 and/or wherein the heterologous nucleic acid sequence encodes FLNC (e.g., a polypeptide represented by UniProt Accession number Q14315 or a polypeptide comprising an amino acid sequence having at least 95% sequence identity thereto).
1328. The method of any one of embodiments 431 to 434, wherein the disease or condition is Myofibrillar Myopathy 6 and/or wherein the heterologous nucleic acid sequence encodes BAG3 (e.g., a polypeptide represented by UniProt Accession number O95817 or a polypeptide comprising an amino acid sequence having at least 95% sequence identity thereto).
1329. The method of any one of embodiments 431 to 434, wherein the disease or condition is Myofibrillar Myopathy 7 and/or wherein the heterologous nucleic acid sequence encodes KY (e.g., a polypeptide represented by UniProt Accession number Q8NBH2 or a polypeptide comprising an amino acid sequence having at least 95% sequence identity thereto).
1330. The method of any one of embodiments 431 to 434, wherein the disease or condition is Myofibrillar Myopathy 8 and/or wherein the heterologous nucleic acid sequence encodes PYROXD1 (e.g., a polypeptide represented by UniProt Accession number Q8WU10 or a polypeptide comprising an amino acid sequence having at least 95% sequence identity thereto).
1331. The method of any one of embodiments 431 to 434, wherein the disease or condition is Myofibrillar Myopathy 9 and/or wherein the heterologous nucleic acid sequence encodes TTN (e.g., a polypeptide represented by UniProt Accession number Q8WZ42 or a polypeptide comprising an amino acid sequence having at least 95% sequence identity thereto).
1332. The method of any one of embodiments 431 to 434, wherein the disease or condition is Myofibrillar Myopathy 10 and/or wherein the heterologous nucleic acid sequence encodes SVIL (e.g., a polypeptide represented by UniProt Accession number O95425 or a polypeptide comprising an amino acid sequence having at least 95% sequence identity thereto).
1333. The method of any one of embodiments 431 to 434, wherein the disease or condition is Myofibrillar Myopathy 11 and/or wherein the heterologous nucleic acid sequence encodes UNC45B (e.g., a polypeptide represented by UniProt Accession number Q8IWX7 or a polypeptide comprising an amino acid sequence having at least 95% sequence identity thereto).
1334. The method of any one of embodiments 431 to 434, wherein the disease or condition is Myofibrillar Myopathy 12 and/or wherein the heterologous nucleic acid sequence encodes MYL2 (e.g., a polypeptide represented by UniProt Accession number Q99972 or a polypeptide comprising an amino acid sequence having at least 95% sequence identity thereto).
1335. The method of any one of embodiments 431 to 434, wherein the disease or condition is Myotonic dystrophy Type 1 (Steinert Disease) and/or wherein the heterologous nucleic acid sequence encodes Myotonin-protein kinase (DMPK) (e.g., a polypeptide represented by UniProt Accession number Q09013 or a polypeptide comprising an amino acid sequence having at least 95% sequence identity thereto).
1336. The method of any one of embodiments 431 to 434, wherein the disease or condition is Myotonic dystrophy Type 2 and/or wherein the heterologous nucleic acid sequence encodes CNBP (e.g., a polypeptide represented by UniProt Accession number P62633 or a polypeptide comprising an amino acid sequence having at least 95% sequence identity thereto).
1337. The method of any one of embodiments 431 to 434, wherein the disease or condition is Myotubular Myopathy and/or wherein the heterologous nucleic acid sequence encodes MTM1 (e.g., a polypeptide represented by UniProt Accession number Q13496 or a polypeptide comprising an amino acid sequence having at least 95% sequence identity thereto).
1338. The method of any one of embodiments 431 to 434, wherein the disease or condition is Nemaline Myopathy 1 and/or wherein the heterologous nucleic acid sequence encodes TPM3 (e.g., a polypeptide represented by UniProt Accession number P06753 or a polypeptide comprising an amino acid sequence having at least 95% sequence identity thereto).
1339. The method of any one of embodiments 431 to 434, wherein the disease or condition is Nemaline Myopathy 2 and/or wherein the heterologous nucleic acid sequence encodes NEB (e.g., a polypeptide represented by UniProt Accession number P20929 or a polypeptide comprising an amino acid sequence having at least 95% sequence identity thereto).
1340. The method of any one of embodiments 431 to 434, wherein the disease or condition is Nemaline Myopathy 5A, 5B, 5C and/or wherein the heterologous nucleic acid sequence encodes TNNT1 (e.g., a polypeptide represented by UniProt Accession number P13805 or a polypeptide comprising an amino acid sequence having at least 95% sequence identity thereto).
1341. The method of any one of embodiments 431 to 434, wherein the disease or condition is Nemaline Myopathy 3 and/or wherein the heterologous nucleic acid sequence encodes ACTA1 (e.g., a polypeptide represented by UniProt Accession number P68133 or a polypeptide comprising an amino acid sequence having at least 95% sequence identity thereto).
1342. The method of any one of embodiments 431 to 434, wherein the disease or condition is Nemaline Myopathy 6 and/or wherein the heterologous nucleic acid sequence encodes KBTBD13 (e.g., a polypeptide represented by UniProt Accession number C9JR72 or a polypeptide comprising an amino acid sequence having at least 95% sequence identity thereto).
1343. The method of any one of embodiments 431 to 434, wherein the disease or condition is Nemaline Myopathy 4 and/or wherein the heterologous nucleic acid sequence encodes TPM2 (e.g., a polypeptide represented by UniProt Accession number P07951 or a polypeptide comprising an amino acid sequence having at least 95% sequence identity thereto).
1344. The method of any one of embodiments 431 to 434, wherein the disease or condition is Nemaline Myopathy 7 and/or wherein the heterologous nucleic acid sequence encodes CFL2 (e.g., a polypeptide represented by UniProt Accession number Q9Y281 or a polypeptide comprising an amino acid sequence having at least 95% sequence identity thereto).
1345. The method of any one of embodiments 431 to 434, wherein the disease or condition is Nemaline Myopathy 8 and/or wherein the heterologous nucleic acid sequence encodes KLHL40 (e.g., a polypeptide represented by UniProt Accession number Q2TBA0 or a polypeptide comprising an amino acid sequence having at least 95% sequence identity thereto).
1346. The method of any one of embodiments 431 to 434, wherein the disease or condition is Nemaline Myopathy 9 and/or wherein the heterologous nucleic acid sequence encodes KLHL41 (e.g., a polypeptide represented by UniProt Accession number O60662 or a polypeptide comprising an amino acid sequence having at least 95% sequence identity thereto).
1347. The method of any one of embodiments 431 to 434, wherein the disease or condition is Nemaline Myopathy 10 and/or wherein the heterologous nucleic acid sequence encodes LMOD3 (e.g., a polypeptide represented by UniProt Accession number Q0VAK6 or a polypeptide comprising an amino acid sequence having at least 95% sequence identity thereto).
1348. The method of any one of embodiments 431 to 434, wherein the disease or condition is Nonaka Distal Myopathy and/or wherein the heterologous nucleic acid sequence encodes GNE (e.g., a polypeptide represented by UniProt Accession number Q9Y223 or a polypeptide comprising an amino acid sequence having at least 95% sequence identity thereto).
1349. The method of any one of embodiments 431 to 434, wherein the disease or condition is Oculopharynggeal muscular dystrophy and/or wherein the heterologous nucleic acid sequence encodes PABPN1 (e.g., a polypeptide represented by UniProt Accession number Q86U42 or a polypeptide comprising an amino acid sequence having at least 95% sequence identity thereto).
1350. The method of any one of embodiments 431 to 434, wherein the disease or condition is Omithine Transcarbamylase deficiency and/or wherein the heterologous nucleic acid sequence encodes OTC (e.g., a polypeptide represented by UniProt Accession number P00480 or a polypeptide comprising an amino acid sequence having at least 95% sequence identity thereto).
1351. The method of any one of embodiments 431 to 434, wherein the disease or condition is Paramyotonia Congenita and/or wherein the heterologous nucleic acid sequence encodes SCN4A (e.g., a polypeptide represented by UniProt Accession number P35499 or a polypeptide comprising an amino acid sequence having at least 95% sequence identity thereto).
1352. The method of any one of embodiments 431 to 434, wherein the disease or condition is Periodic Paralysis, hypokalemic and/or wherein the heterologous nucleic acid sequence encodes CACNA1S (e.g., a polypeptide represented by UniProt Accession number Q13698 or a polypeptide comprising an amino acid sequence having at least 95% sequence identity thereto).
1353. The method of any one of embodiments 431 to 434, wherein the disease or condition is Periodic Paralysis, hyperkalemic and/or wherein the heterologous nucleic acid sequence encodes SCN4A (e.g., a polypeptide represented by UniProt Accession number P35499 or a polypeptide comprising an amino acid sequence having at least 95% sequence identity thereto).
1354. The method of any one of embodiments 431 to 434, wherein the disease or condition is Phosphoglycerate Kinase Deficiency and/or wherein the heterologous nucleic acid sequence encodes PGK1 (e.g., a polypeptide represented by UniProt Accession number P00558 or a polypeptide comprising an amino acid sequence having at least 95% sequence identity thereto).
1355. The method of any one of embodiments 431 to 434, wherein the disease or condition is Polymyositis and/or wherein the heterologous nucleic acid sequence encodes PMSCL2 (e.g., a polypeptide represented by UniProt Accession number Q01780 or a polypeptide comprising an amino acid sequence having at least 95% sequence identity thereto).
1356. The method of any one of embodiments 431 to 434, wherein the disease or condition is Polymyositis and/or wherein the heterologous nucleic acid sequence encodes PMSCL1 (e.g., a polypeptide represented by UniProt Accession number Q06265 or a polypeptide comprising an amino acid sequence having at least 95% sequence identity thereto).
1357. The method of any one of embodiments 431 to 434, wherein the disease or condition is Progressive External Ophthalmoplegia and/or wherein the heterologous nucleic acid sequence encodes POLG (e.g., a polypeptide represented by UniProt Accession number P54098 or a polypeptide comprising an amino acid sequence having at least 95% sequence identity thereto).
1358. The method of any one of embodiments 431 to 434, wherein the disease or condition is Progressive External Ophthalmoplegia and/or wherein the heterologous nucleic acid sequence encodes POLG2 (e.g., a polypeptide represented by UniProt Accession number Q9UHN1 or a polypeptide comprising an amino acid sequence having at least 95% sequence identity thereto).
1359. The method of any one of embodiments 431 to 434, wherein the disease or condition is Progressive External Ophthalmoplegia and/or wherein the heterologous nucleic acid sequence encodes SLC25A4 (e.g., a polypeptide represented by UniProt Accession number P12235 or a polypeptide comprising an amino acid sequence having at least 95% sequence identity thereto).
1360. The method of any one of embodiments 431 to 434, wherein the disease or condition is Progressive External Ophthalmoplegia and/or wherein the heterologous nucleic acid sequence encodes TWNK (e.g., a polypeptide represented by UniProt Accession number Q96RR1 or a polypeptide comprising an amino acid sequence having at least 95% sequence identity thereto).
1361. The method of any one of embodiments 431 to 434, wherein the disease or condition is Spinal Muscular Atrophy type 3—Kugelberg-Welander Disease and/or wherein the heterologous nucleic acid sequence encodes Survival motor neuron protein (SMN), SMN1 (e.g., a polypeptide represented by UniProt Accession number Q16637 or a polypeptide comprising an amino acid sequence having at least 95% sequence identity thereto).
1362. The method of any one of embodiments 431 to 434, wherein the disease or condition is Thomsen Disease (Myotonia Congenita (autosomal dominant)) and/or wherein the heterologous nucleic acid sequence encodes CLCN1 (e.g., a polypeptide represented by UniProt Accession number P35523 or a polypeptide comprising an amino acid sequence having at least 95% sequence identity thereto).
1363. The method of any one of embodiments 431 to 434, wherein the disease or condition is Walker-Warburg Syndrome and/or wherein the heterologous nucleic acid sequence encodes POMT1 (e.g., a polypeptide represented by UniProt Accession number Q9Y6A1 or a polypeptide comprising an amino acid sequence having at least 95% sequence identity thereto).
1364. The method of any one of embodiments 431 to 434, wherein the disease or condition is X-linked myotubular myopathy and/or wherein the heterologous nucleic acid sequence encodes Myotubularin (MTM1) (e.g., a polypeptide represented by UniProt Accession number Q13496 or a polypeptide comprising an amino acid sequence having at least 95% sequence identity thereto).
1365. The method of any one of embodiments 431 to 434, wherein the disease or condition is Werdnig-Hoffmann Disease—Spinal Muscular Atrophy type 1 and/or wherein the heterologous nucleic acid sequence encodes SMN1 (e.g., a polypeptide represented by UniProt Accession number Q16637 or a polypeptide comprising an amino acid sequence having at least 95% sequence identity thereto).
1366. The method of any one of embodiments 431 to 434, wherein the disease or condition is Amyotrophic lateral sclerosis and/or wherein the heterologous nucleic acid sequence encodes an antisense oligonucleotide targeting Superoxide dismutase 1 (SOD 1) (e.g., targeting an RNA encoding a polypeptide represented by UniProt Accession number P00441 or a polypeptide comprising an amino acid sequence having at least 95% sequence identity thereto).
1367. The method of any one of embodiments 431 to 434, wherein the disease or condition is Amyotrophic lateral sclerosis and/or wherein the heterologous nucleic acid sequence encodes an antisense oligonucleotide targeting C9orf72 (e.g., targeting an RNA encoding a polypeptide represented by UniProt Accession number Q96LT7 or a polypeptide comprising an amino acid sequence having at least 95% sequence identity thereto).
1368. The method of any one of embodiments 431 to 434, wherein the disease or condition is Huntington's disease and/or wherein the heterologous nucleic acid sequence encodes an antisense oligonucleotide targeting Huntingtin (HTT) (e.g., targeting an RNA encoding a polypeptide represented by UniProt Accession number P42858 or a polypeptide comprising an amino acid sequence having at least 95% sequence identity thereto).
1369. The method of any one of embodiments 431 to 434, wherein the disease or condition is Alzheimer's disease and/or wherein the heterologous nucleic acid sequence encodes an antisense oligonucleotide targeting Sortilin-related receptor (SORL1) (e.g., targeting an RNA encoding a polypeptide represented by UniProt Accession number Q92673 or a polypeptide comprising an amino acid sequence having at least 95% sequence identity thereto).
1370. The method of any one of embodiments 431 to 434, wherein the disease or condition is Alzheimer's disease and/or wherein the heterologous nucleic acid sequence encodes an antisense oligonucleotide targeting Neutral sphingomyelinase (N-SMase; SMPD2) (e.g., targeting an RNA encoding a polypeptide represented by UniProt Accession number O60906 or a polypeptide comprising an amino acid sequence having at least 95% sequence identity thereto).
1371. The method of any one of embodiments 431 to 434, wherein the disease or condition is Spinocerebellar ataxia type 2 and/or wherein the heterologous nucleic acid sequence encodes an antisense oligonucleotide targeting Ataxin-2 (ATXN2) (e.g., targeting an RNA encoding a polypeptide represented by UniProt Accession number Q99700 or a polypeptide comprising an amino acid sequence having at least 95% sequence identity thereto).
1372. The method of any one of embodiments 431 to 434, wherein the disease or condition is Parkinson's Disease and/or wherein the heterologous nucleic acid sequence encodes an antisense oligonucleotide targeting human SNCA (e.g., targeting an RNA encoding a polypeptide represented by UniProt Accession number P37840 or a polypeptide comprising an amino acid sequence having at least 95% sequence identity thereto).
1373. The method of any one of embodiments 431 to 434, wherein the disease or condition is Fragile X syndrome and/or wherein the heterologous nucleic acid sequence encodes FMR1 (e.g., a polypeptide represented by UniProt Accession number Q06787 or a polypeptide comprising an amino acid sequence having at least 95% sequence identity thereto).
1374. The method of embodiment 431 or embodiment 432, wherein the disease or condition is a muscle disease or condition.
1375. The method of embodiment 1374, wherein the disease or condition is a skeletal muscle disease or condition.
1376. The method of embodiment 1374, wherein the disease or condition is a cardiac muscle disease or condition.
1377. The method of any one of embodiments 431 to 432 and 1374 to 1376, wherein the disease or condition is Acid Maltase Deficiency (AMD), Amyotrophic Lateral Sclerosis (ALS), Andersen-Tawil Syndrome, Barth syndrome (TAZ), Becker Muscular Dystrophy (BMD), Becker Myotonia Congenita, Bethlem Myopathy, Bulbospinal Muscular Atrophy (Spinal—Bulbar Muscular Atrophy), Carnitine Deficiency, Carnitine Palmityl Transferase Deficiency (CPT Deficiency), Central Core Disease (CCD), Centronuclear Myopathy, Charcot-Marie-Tooth Disease (CMT), Congenital Muscular Dystrophy (CMD), Congenital Myasthenic Syndromes (CMS), Congenital Myotonic Dystrophy, Cori Disease (Debrancher Enzyme Deficiency), Danon disease, Debrancher Enzyme Deficiency, Dejerine-Sottas Disease (DSD), Dermatomyositis (DM), Distal Muscular Dystrophy (DD), Distal myopathy with anterior tibial onset, Duchenne Muscular Dystrophy (DMD), Dystrophia Myotonica (Myotonic Muscular Dystrophy), Emery-Dreifuss Muscular Dystrophy (EDMD), Endocrine Myopathies, Eulenberg Disease (Paramyotonia Congenita), Facioscapulohumeral Muscular Dystrophy (FSH or FSHD), Finnish (Tibial) Distal Myopathy, Forbes Disease (Debrancher Enzyme Deficiency), Friedreich's Ataxia (FA), Fukuyama Congenital Muscular Dystrophy, Glycogenosis Type 10, Glycogenosis Type 11, Glycogenosis Type 2, Glycogenosis Type 3, Glycogenosis Type 5, Glycogenosis Type 7, Glycogenosis Type 9, Gowers-Laing Distal Myopathy, Hauptmann-Thanheuser MD (Emery—Dreifuss Muscular Dystrophy), Hereditary Inclusion-Body Myositis, Hereditary Motor and Sensory Neuropathy (Charcot-Marie-Tooth Disease), Hyperthyroid Myopathy, Hypothyroid Myopathy, Inclusion-Body Myositis (IBM), Inherited Myopathies, Integrin-Deficient Congenital Muscular Dystrophy, Kennedy Disease (Spinal-Bulbar Muscular Atrophy), Kugelberg-Welander Disease (Spinal Muscular Atrophy), Lactate Dehydrogenase Deficiency, Lambert-Eaton Myasthenic Syndrome (LEMS), Limb-Girdle Muscular Dystrophy (LGMD), Lou Gehrig's Disease (Amyotrophic Lateral Sclerosis), McArdle Disease (Phosphorylase Deficiency), Merosin-Deficient Congenital Muscular Dystrophy, Metabolic Diseases of Muscle, Mitochondrial Myopathy, Miyoshi myopathy, Miyoshi Distal Myopathy, Motor Neurone Disease, Muscle-Eye-Brain Disease, Myasthenia Gravis (MG), Myoadenylate Deaminase Deficiency, Myofibrillar Myopathy, Myophosphorylase Deficiency, Myotonia Congenita (MC), Myotonic Muscular Dystrophy (MMD), Myotubular Myopathy (MTM or MM), Nemaline Myopathy, Nonaka Distal Myopathy, Oculopharyngeal Muscular Dystrophy (OPMD), Paramyotonia Congenita, Pearson Syndrome, Periodic Paralysis, Peroneal Muscular Atrophy (Charcot-Marie-Tooth Disease), Phosphofructokinase Deficiency, Phosphoglycerate Kinase Deficiency, Phosphoglycerate Mutase Deficiency, Phosphorylase Deficiency, Phosphorylase Deficiency, Polymyositis (PM), Pompe Disease (Acid Maltase Deficiency), Primary merosin deficiency (LAMA2), Progressive External Ophthalmoplegia (PEO), Rod Body Disease (Nemaline Myopathy), Spinal Muscular Atrophy (SMA), Spinal-Bulbar Muscular Atrophy (SBMA), Steinert Disease (Myotonic Muscular Dystrophy), Tarui Disease (Phosphofructokinase Deficiency), Thomsen Disease (Myotonia Congenita), Ullrich Congenital Muscular Dystrophy, Walker-Warburg Syndrome (Congenital Muscular Dystrophy), Welander Distal Myopathy, Werdnig-Hoffmann Disease (Spinal Muscular Atrophy), or ZASP-Related Myopathy.
1378. The method of any one of embodiments 431 to 432 and 1374 to 1377, wherein the disease or condition is Acid Maltase Deficiency (AMD).
1379. The method of any one of embodiments 431 to 432 and 1374 to 1377, wherein the disease or condition is Amyotrophic Lateral Sclerosis (ALS).
1380. The method of any one of embodiments 431 to 432 and 1374 to 1377, wherein the disease or condition is Andersen-Tawil Syndrome.
1381. The method of any one of embodiments 431 to 432 and 1374 to 1377, wherein the disease or condition is Barth syndrome (TAZ).
1382. The method of any one of embodiments 431 to 432 and 1374 to 1377, wherein the disease or condition is Becker Muscular Dystrophy (BMD).
1383. The method of any one of embodiments 431 to 432 and 1374 to 1377, wherein the disease or condition is Becker Myotonia Congenita.
1384. The method of any one of embodiments 431 to 432 and 1374 to 1377, wherein the disease or condition is Bethlem Myopathy.
1385. The method of any one of embodiments 431 to 432 and 1374 to 1377, wherein the disease or condition is Bulbospinal Muscular Atrophy (Spinal—Bulbar Muscular Atrophy).
1386. The method of any one of embodiments 431 to 432 and 1374 to 1377, wherein the disease or condition is Carnitine Deficiency.
1387. The method of any one of embodiments 431 to 432 and 1374 to 1377, wherein the disease or condition is Carnitine Palmityl Transferase Deficiency (CPT Deficiency).
1388. The method of any one of embodiments 431 to 432 and 1374 to 1377, wherein the disease or condition is Central Ctoe Disease (CCD).
1389. The method of any one of embodiments 431 to 432 and 1374 to 1377, wherein the disease or condition is Centronuclear Myopathy.
1390. The method of any one of embodiments 431 to 432 and 1374 to 1377, wherein the disease or condition is Charcot-Marie-Tooth Disease (CMT).
1391. The method of any one of embodiments 431 to 432 and 1374 to 1377, wherein the disease or condition is Congenital Muscular Dystrophy (CMD).
1392. The method of any one of embodiments 431 to 432 and 1374 to 1377, wherein the disease or condition is Congenital Myasthenic Syndromes (CMS).
1393. The method of any one of embodiments 431 to 432 and 1374 to 1377, wherein the disease or condition is Congenital Myotonic Dystrophy.
1394. The method of any one of embodiments 431 to 432 and 1374 to 1377, wherein the disease or condition is Ctoi Disease (Debrancher Enzyme Deficiency).
1395. The method of any one of embodiments 431 to 432 and 1374 to 1377, wherein the disease or condition is Danon disease.
1396. The method of any one of embodiments 431 to 432 and 1374 to 1377, wherein the disease or condition is Debrancher Enzyme Deficiency.
1397. The method of any one of embodiments 431 to 432 and 1374 to 1377, wherein the disease or condition is Dejerine-Sottas Disease (DSD).
1398. The method of any one of embodiments 431 to 432 and 1374 to 1377, wherein the disease or condition is Dermatomyositis (DM).
1399. The method of any one of embodiments 431 to 432 and 1374 to 1377, wherein the disease or condition is Distal Muscular Dystrophy (DD).
1400. The method of any one of embodiments 431 to 432 and 1374 to 1377, wherein the disease or condition is Distal myopathy with anterito tibial onset.
1401. The method of any one of embodiments 431 to 432 and 1374 to 1377, wherein the disease or condition is Duchenne Muscular Dystrophy (DMD).
1402. The method of any one of embodiments 431 to 432 and 1374 to 1377, wherein the disease or condition is Dystrophia Myotonica (Myotonic Muscular Dystrophy).
1403. The method of any one of embodiments 431 to 432 and 1374 to 1377, wherein the disease or condition is Emery-Dreifuss Muscular Dystrophy (EDMD).
1404. The method of any one of embodiments 1332 to 1224, wherein the disease or condition is an endocrine myopathy.
1405. The method of any one of embodiments 431 to 432 and 1374 to 1377, wherein the disease or condition is Eulenberg Disease (Paramyotonia Congenita).
1406. The method of any one of embodiments 431 to 432 and 1374 to 1377, wherein the disease or condition is Facioscapulohumeral Muscular Dystrophy (FSH to FSHD).
1407. The method of any one of embodiments 431 to 432 and 1374 to 1377, wherein the disease or condition is Finnish (Tibial) Distal Myopathy.
1408. The method of any one of embodiments 431 to 432 and 1374 to 1377, wherein the disease or condition is Ftobes Disease (Debrancher Enzyme Deficiency).
1409. The method of any one of embodiments 431 to 432 and 1374 to 1377, wherein the disease or condition is Friedreich's Ataxia (FA).
1410. The method of any one of embodiments 431 to 432 and 1374 to 1377, wherein the disease or condition is Fukuyama Congenital Muscular Dystrophy.
1411. The method of any one of embodiments 431 to 432 and 1374 to 1377, wherein the disease or condition is Glycogenosis Type 10.
1412. The method of any one of embodiments 431 to 432 and 1374 to 1377, wherein the disease or condition is Glycogenosis Type 11.
1413. The method of any one of embodiments 431 to 432 and 1374 to 1377, wherein the disease or condition is Glycogenosis Type 2.
1414. The method of any one of embodiments 431 to 432 and 1374 to 1377, wherein the disease or condition is Glycogenosis Type 3.
1415. The method of any one of embodiments 431 to 432 and 1374 to 1377, wherein the disease or condition is Glycogenosis Type 5.
1416. The method of any one of embodiments 431 to 432 and 1374 to 1377, wherein the disease or condition is Glycogenosis Type 7.
1417. The method of any one of embodiments 431 to 432 and 1374 to 1377, wherein the disease or condition is Glycogenosis Type 9.
1418. The method of any one of embodiments 431 to 432 and 1374 to 1377, wherein the disease or condition is Gowers-Laing Distal Myopathy.
1419. The method of any one of embodiments 431 to 432 and 1374 to 1377, wherein the disease or condition is Hauptmann-Thanheuser MD (Emery—Dreifuss Muscular Dystrophy).
1420. The method of any one of embodiments 431 to 432 and 1374 to 1377, wherein the disease or condition is Hereditary Inclusion-Body Myositis.
1421. The method of any one of embodiments 431 to 432 and 1374 to 1377, wherein the disease or condition is Hereditary Motto and Senstoy Neuropathy (Charcot-Marie-Tooth Disease).
1422. The method of any one of embodiments 431 to 432 and 1374 to 1377, wherein the disease or condition is Hyperthyroid Myopathy.
1423. The method of any one of embodiments 431 to 432 and 1374 to 1377, wherein the disease or condition is Hypothyroid Myopathy.
1424. The method of any one of embodiments 431 to 432 and 1374 to 1377, wherein the disease or condition is Inclusion-Body Myositis (IBM).
1425. The method of any one of embodiments 431 to 432 and 1374 to 1377, wherein the disease or condition is an inherited myopathy.
1426. The method of any one of embodiments 431 to 432 and 1374 to 1377, wherein the disease or condition is Integrin-Deficient Congenital Muscular Dystrophy.
1427. The method of any one of embodiments 431 to 432 and 1374 to 1377, wherein the disease or condition is Kennedy Disease (Spinal-Bulbar Muscular Atrophy).
1428. The method of any one of embodiments 431 to 432 and 1374 to 1377, wherein the disease or condition is Kugelberg-Welander Disease (Spinal Muscular Atrophy).
1429. The method of any one of embodiments 431 to 432 and 1374 to 1377, wherein the disease or condition is Lactate Dehydrogenase Deficiency.
1430. The method of any one of embodiments 431 to 432 and 1374 to 1377, wherein the disease or condition is Lambert-Eaton Myasthenic Syndrome (LEMS).
1431. The method of any one of embodiments 431 to 432 and 1374 to 1377, wherein the disease or condition is Limb-Girdle Muscular Dystrophy (LGMD).
1432. The method of any one of embodiments 431 to 432 and 1374 to 1377, wherein the disease or condition is Lou Gehrig's Disease (Amyotrophic Lateral Sclerosis).
1433. The method of any one of embodiments 431 to 432 and 1374 to 1377, wherein the disease or condition is McArdle Disease (Phosphtoylase Deficiency).
1434. The method of any one of embodiments 431 to 432 and 1374 to 1377, wherein the disease or condition is Merosin-Deficient Congenital Muscular Dystrophy.
1435. The method of any one of embodiments 431 to 432 and 1374 to 1377, wherein the disease or condition is Metabolic Diseases of Muscle.
1436. The method of any one of embodiments 431 to 432 and 1374 to 1377, wherein the disease or condition is Mitochondrial Myopathy.
1437. The method of any one of embodiments 431 to 432 and 1374 to 1377, wherein the disease or condition is Miyoshi myopathy.
1438. The method of any one of embodiments 431 to 432 and 1374 to 1377, wherein the disease or condition is Miyoshi Distal Myopathy.
1439. The method of any one of embodiments 431 to 432 and 1374 to 1377, wherein the disease or condition is Motto Neurone Disease.
1440. The method of any one of embodiments 431 to 432 and 1374 to 1377, wherein the disease or condition is Muscle-Eye-Brain Disease.
1441. The method of any one of embodiments 431 to 432 and 1374 to 1377, wherein the disease or condition is Myasthenia Gravis (MG).
1442. The method of any one of embodiments 431 to 432 and 1374 to 1377, wherein the disease or condition is Myoadenylate Deaminase Deficiency.
1443. The method of any one of embodiments 431 to 432 and 1374 to 1377, wherein the disease or condition is Myofibrillar Myopathy.
1444. The method of any one of embodiments 431 to 432 and 1374 to 1377, wherein the disease or condition is Myophosphtoylase Deficiency.
1445. The method of any one of embodiments 431 to 432 and 1374 to 1377, wherein the disease or condition is Myotonia Congenita (MC).
1446. The method of any one of embodiments 431 to 432 and 1374 to 1377, wherein the disease or condition is Myotonic Muscular Dystrophy (MMD).
1447. The method of any one of embodiments 431 to 432 and 1374 to 1377, wherein the disease or condition is Myotubular Myopathy (MTM to MM).
1448. The method of any one of embodiments 431 to 432 and 1374 to 1377, wherein the disease or condition is Nemaline Myopathy.
1449. The method of any one of embodiments 431 to 432 and 1374 to 1377, wherein the disease or condition is Nonaka Distal Myopathy.
1450. The method of any one of embodiments 431 to 432 and 1374 to 1377, wherein the disease or condition is Oculopharyngeal Muscular Dystrophy (OPMD).
1451. The method of any one of embodiments 431 to 432 and 1374 to 1377, wherein the disease or condition is Paramyotonia Congenita.
1452. The method of any one of embodiments 431 to 432 and 1374 to 1377, wherein the disease or condition is Pearson Syndrome.
1453. The method of any one of embodiments 431 to 432 and 1374 to 1377, wherein the disease or condition is Periodic Paralysis.
1454. The method of any one of embodiments 431 to 432 and 1374 to 1377, wherein the disease or condition is Peroneal Muscular Atrophy (Charcot-Marie-Tooth Disease).
1455. The method of any one of embodiments 431 to 432 and 1374 to 1377, wherein the disease or condition is Phosphofructokinase Deficiency.
1456. The method of any one of embodiments 431 to 432 and 1374 to 1377, wherein the disease or condition is Phosphoglycerate Kinase Deficiency.
1457. The method of any one of embodiments 431 to 432 and 1374 to 1377, wherein the disease or condition is Phosphoglycerate Mutase Deficiency.
1458. The method of any one of embodiments 431 to 432 and 1374 to 1377, wherein the disease or condition is Phosphtoylase Deficiency.
1459. The method of any one of embodiments 431 to 432 and 1374 to 1377, wherein the disease or condition is Polymyositis (PM).
1460. The method of any one of embodiments 431 to 432 and 1374 to 1377, wherein the disease or condition is Pompe Disease (Acid Maltase Deficiency).
1461. The method of any one of embodiments 431 to 432 and 1374 to 1377, wherein the disease or condition is Primary merosin deficiency (LAMA2).
1462. The method of any one of embodiments 431 to 432 and 1374 to 1377, wherein the disease or condition is Progressive External Ophthalmoplegia (PEO).
1463. The method of any one of embodiments 431 to 432 and 1374 to 1377, wherein the disease or condition is Rod Body Disease (Nemaline Myopathy).
1464. The method of any one of embodiments 431 to 432 and 1374 to 1377, wherein the disease or condition is Muscular Atrophy (SMA).
1465. The method of any one of embodiments 431 to 432 and 1374 to 1377, wherein the disease or condition is Spinal-Bulbar Muscular Atrophy (SBMA).
1466. The method of any one of embodiments 431 to 432 and 1374 to 1377, wherein the disease or condition is Steinert Disease (Myotonic Muscular Dystrophy).
1467. The method of any one of embodiments 431 to 432 and 1374 to 1377, wherein the disease or condition is Tarui Disease (Phosphofructokinase Deficiency).
1468. The method of any one of embodiments 431 to 432 and 1374 to 1377, wherein the disease or condition is Thomsen Disease (Myotonia Congenita).
1469. The method of any one of embodiments 431 to 432 and 1374 to 1377, wherein the disease or condition is Ullrich Congenital Muscular Dystrophy.
1470. The method of any one of embodiments 431 to 432 and 1374 to 1377, wherein the disease or condition is Walker-Warburg Syndrome (Congenital Muscular Dystrophy).
1471. The method of any one of embodiments 431 to 432 and 1374 to 1377, wherein the disease or condition is Welander Distal Myopathy.
1472. The method of any one of embodiments 431 to 432 and 1374 to 1377, wherein the disease or condition is Werdnig-Hoffmann Disease (Spinal Muscular Atrophy).
1473. The method of any one of embodiments 431 to 432 and 1374 to 1377, wherein the disease or condition is ZASP-Related Myopathy.
1474. The method of any one of embodiments 431 to 432 and 1374 to 1377, wherein the disease or condition is Advanced heart failure and/or wherein the heterologous nucleic acid sequence encodes SERCA2a, ATP2A2 (e.g., a polypeptide represented by UniProt Accession number P16615 to a polypeptide comprising an amino acid sequence having at least 95% sequence identity thereto).
1475. The method of any one of embodiments 431 to 432 and 1374 to 1377, wherein the disease or condition is Amyotrophic lateral sclerosis (ALS) (Lou Gehrig's disease) and/or wherein the heterologous nucleic acid sequence encodes Superoxide dismutase-1 (SOD1) (e.g., a polypeptide represented by UniProt Accession number P00441 to a polypeptide comprising an amino acid sequence having at least 95% sequence identity thereto).
1476. The method of any one of embodiments 431 to 432 and 1374 to 1377, wherein the disease or condition is Andersen-Tawil Syndrome and/or wherein the heterologous nucleic acid sequence encodes KCNJ2 (e.g., a polypeptide represented by UniProt Accession number P63252 to a polypeptide comprising an amino acid sequence having at least 95% sequence identity thereto).
1477. The method of any one of embodiments 431 to 432 and 1374 to 1377, wherein the disease or condition is Barth syndrome and/or wherein the heterologous nucleic acid sequence encodes TAFAZZIN (e.g., a polypeptide represented by UniProt Accession number Q16635 to a polypeptide comprising an amino acid sequence having at least 95% sequence identity thereto).
1478. The method of any one of embodiments 431 to 432 and 1374 to 1377, wherein the disease or condition is Becker Muscular Dystrophy (BMD) and/or wherein the heterologous nucleic acid sequence encodes DMD (e.g., a polypeptide represented by UniProt Accession number P11532 to a polypeptide comprising an amino acid sequence having at least 95% sequence identity thereto).
1479. The method of any one of embodiments 431 to 432 and 1374 to 1377, wherein the disease or condition is Becker Myotonia Congenita and/or wherein the heterologous nucleic acid sequence encodes CLCN1 (e.g., a polypeptide represented by UniProt Accession number P35523 to a polypeptide comprising an amino acid sequence having at least 95% sequence identity thereto).
1480. The method of any one of embodiments 431 to 432 and 1374 to 1377, wherein the disease or condition is Bethlem Myopathy and/or wherein the heterologous nucleic acid sequence encodes COL6A3 (e.g., a polypeptide represented by UniProt Accession number P12111 to a polypeptide comprising an amino acid sequence having at least 95% sequence identity thereto).
1481. The method of any one of embodiments 431 to 432 and 1374 to 1377, wherein the disease or condition is Bethlem Myopathy and/or wherein the heterologous nucleic acid sequence encodes COL6A2 (e.g., a polypeptide represented by UniProt Accession number P12110 to a polypeptide comprising an amino acid sequence having at least 95% sequence identity thereto).
1482. The method of any one of embodiments 431 to 432 and 1374 to 1377, wherein the disease or condition is Bethlem Myopathy and/or wherein the heterologous nucleic acid sequence encodes COL6A1 (e.g., a polypeptide represented by UniProt Accession number P12109 to a polypeptide comprising an amino acid sequence having at least 95% sequence identity thereto).
1483. The method of any one of embodiments 431 to 432 and 1374 to 1377, wherein the disease or condition is Bulbospinal Muscular Atrophy and/or wherein the heterologous nucleic acid sequence encodes AR (e.g., a polypeptide represented by UniProt Accession number P10275 to a polypeptide comprising an amino acid sequence having at least 95% sequence identity thereto).
1484. The method of any one of embodiments 431 to 432 and 1374 to 1377, wherein the disease or condition is Carnitine Deficiency, systemic primary and/or wherein the heterologous nucleic acid sequence encodes SLC22A5 (e.g., a polypeptide represented by UniProt Accession number 076082 to a polypeptide comprising an amino acid sequence having at least 95% sequence identity thereto).
1485. The method of any one of embodiments 431 to 432 and 1374 to 1377, wherein the disease or condition is Carnitine Palmityl Transferase Deficiency, type 1 and/or wherein the heterologous nucleic acid sequence encodes CPT1A (e.g., a polypeptide represented by UniProt Accession number P50416 to a polypeptide comprising an amino acid sequence having at least 95% sequence identity thereto).
1486. The method of any one of embodiments 431 to 432 and 1374 to 1377, wherein the disease or condition is Carnitine Palmityl Transferase Deficiency, type 2 and/or wherein the heterologous nucleic acid sequence encodes CPT2 (e.g., a polypeptide represented by UniProt Accession number P23786 to a polypeptide comprising an amino acid sequence having at least 95% sequence identity thereto).
1487. The method of any one of embodiments 431 to 432 and 1374 to 1377, wherein the disease or condition is Catecholaminergic polymtophic ventricular tachycardia 2 (CPVT2) and/or wherein the heterologous nucleic acid sequence encodes Calsequestrin-2 (CASQ2) (e.g., a polypeptide represented by UniProt Accession number O14958 to a polypeptide comprising an amino acid sequence having at least 95% sequence identity thereto).
1488. The method of any one of embodiments 431 to 432 and 1374 to 1377, wherein the disease or condition is Central Ctoe Disease (congenital myopathy, type 1A) and/or wherein the heterologous nucleic acid sequence encodes RYR1 (e.g., a polypeptide represented by UniProt Accession number P21817 to a polypeptide comprising an amino acid sequence having at least 95% sequence identity thereto).
1489. The method of any one of embodiments 431 to 432 and 1374 to 1377, wherein the disease or condition is Centronuclear Myopathy type 1 and/or wherein the heterologous nucleic acid sequence encodes MTMR14 (e.g., a polypeptide represented by UniProt Accession number Q8NCE2 to a polypeptide comprising an amino acid sequence having at least 95% sequence identity thereto).
1490. The method of any one of embodiments 431 to 432 and 1374 to 1377, wherein the disease or condition is Centronuclear Myopathy type 2 and/or wherein the heterologous nucleic acid sequence encodes DNM2 (e.g., a polypeptide represented by UniProt Accession number O14717 to a polypeptide comprising an amino acid sequence having at least 95% sequence identity thereto).
1491. The method of any one of embodiments 431 to 432 and 1374 to 1377, wherein the disease or condition is Charcot-Marie-Tooth disease and/or wherein the heterologous nucleic acid sequence encodes PMP22 (e.g., a polypeptide represented by UniProt Accession number Q01453 to a polypeptide comprising an amino acid sequence having at least 95% sequence identity thereto).
1492. The method of any one of embodiments 431 to 432 and 1374 to 1377, wherein the disease or condition is Charcot-Marie-Tooth disease and/or wherein the heterologous nucleic acid sequence encodes MPZ (e.g., a polypeptide represented by UniProt Accession number P25189 to a polypeptide comprising an amino acid sequence having at least 95% sequence identity thereto).
1493. The method of any one of embodiments 431 to 432 and 1374 to 1377, wherein the disease or condition is Charcot-Marie-Tooth disease and/or wherein the heterologous nucleic acid sequence encodes DNM2 (e.g., a polypeptide represented by UniProt Accession number P50570 to a polypeptide comprising an amino acid sequence having at least 95% sequence identity thereto).
1494. The method of any one of embodiments 431 to 432 and 1374 to 1377, wherein the disease or condition is Charcot-Marie-Tooth disease and/or wherein the heterologous nucleic acid sequence encodes MFN2 (e.g., a polypeptide represented by UniProt Accession number O95140 to a polypeptide comprising an amino acid sequence having at least 95% sequence identity thereto).
1495. The method of any one of embodiments 431 to 432 and 1374 to 1377, wherein the disease or condition is Charcot-Marie-Tooth disease and/or wherein the heterologous nucleic acid sequence encodes KIF1B (e.g., a polypeptide represented by UniProt Accession number O60333 to a polypeptide comprising an amino acid sequence having at least 95% sequence identity thereto).
1496. The method of any one of embodiments 431 to 432 and 1374 to 1377, wherein the disease or condition is Charcot-Marie-Tooth disease and/or wherein the heterologous nucleic acid sequence encodes SBF2 (e.g., a polypeptide represented by UniProt Accession number Q86WG5 to a polypeptide comprising an amino acid sequence having at least 95% sequence identity thereto).
1497. The method of any one of embodiments 431 to 432 and 1374 to 1377, wherein the disease or condition is Charcot-Marie-Tooth disease and/or wherein the heterologous nucleic acid sequence encodes PNKP (e.g., a polypeptide represented by UniProt Accession number Q96T60 to a polypeptide comprising an amino acid sequence having at least 95% sequence identity thereto).
1498. The method of any one of embodiments 431 to 432 and 1374 to 1377, wherein the disease or condition is Charcot-Marie-Tooth disease and/or wherein the heterologous nucleic acid sequence encodes GDAP1 (e.g., a polypeptide represented by UniProt Accession number Q8TB36 to a polypeptide comprising an amino acid sequence having at least 95% sequence identity thereto).
1499. The method of any one of embodiments 431 to 432 and 1374 to 1377, wherein the disease or condition is Charcot-Marie-Tooth disease and/or wherein the heterologous nucleic acid sequence encodes LMNA (e.g., a polypeptide represented by UniProt Accession number P02545 to a polypeptide comprising an amino acid sequence having at least 95% sequence identity thereto).
1500. The method of any one of embodiments 431 to 432 and 1374 to 1377, wherein the disease or condition is Charcot-Marie-Tooth disease and/or wherein the heterologous nucleic acid sequence encodes FGD4 (e.g., a polypeptide represented by UniProt Accession number Q96M96 to a polypeptide comprising an amino acid sequence having at least 95% sequence identity thereto).
1501. The method of any one of embodiments 431 to 432 and 1374 to 1377, wherein the disease or condition is Charcot-Marie-Tooth disease and/or wherein the heterologous nucleic acid sequence encodes MTMR2 (e.g., a polypeptide represented by UniProt Accession number Q13614 to a polypeptide comprising an amino acid sequence having at least 95% sequence identity thereto).
1502. The method of any one of embodiments 431 to 432 and 1374 to 1377, wherein the disease or condition is Congenital Muscular Dystrophy (Ullrich disease) and/or wherein the heterologous nucleic acid sequence encodes COL6A3 (e.g., a polypeptide represented by UniProt Accession number P12111 to a polypeptide comprising an amino acid sequence having at least 95% sequence identity thereto).
1503. The method of any one of embodiments 431 to 432 and 1374 to 1377, wherein the disease or condition is Congenital Muscular Dystrophy (Ullrich disease) and/or wherein the heterologous nucleic acid sequence encodes COL6A2 (e.g., a polypeptide represented by UniProt Accession number P12110 to a polypeptide comprising an amino acid sequence having at least 95% sequence identity thereto).
1504. The method of any one of embodiments 431 to 432 and 1374 to 1377, wherein the disease or condition is Congenital Muscular Dystrophy (Ullrich disease) and/or wherein the heterologous nucleic acid sequence encodes COL6A1 (e.g., a polypeptide represented by UniProt Accession number P12109 to a polypeptide comprising an amino acid sequence having at least 95% sequence identity thereto).
1505. The method of any one of embodiments 431 to 432 and 1374 to 1377, wherein the disease or condition is Congenital Myasthenic Syndrome and/or wherein the heterologous nucleic acid sequence encodes COLQ (e.g., a polypeptide represented by UniProt Accession number Q9Y215 to a polypeptide comprising an amino acid sequence having at least 95% sequence identity thereto).
1506. The method of any one of embodiments 431 to 432 and 1374 to 1377, wherein the disease or condition is Congenital Myasthenic Syndrome and/or wherein the heterologous nucleic acid sequence encodes AGRN (e.g., a polypeptide represented by UniProt Accession number O00468 to a polypeptide comprising an amino acid sequence having at least 95% sequence identity thereto).
1507. The method of any one of embodiments 431 to 432 and 1374 to 1377, wherein the disease or condition is Congenital Myasthenic Syndrome and/or wherein the heterologous nucleic acid sequence encodes RAPSN (e.g., a polypeptide represented by UniProt Accession number Q13702 to a polypeptide comprising an amino acid sequence having at least 95% sequence identity thereto).
1508. The method of any one of embodiments 431 to 432 and 1374 to 1377, wherein the disease or condition is Congenital Myasthenic Syndrome and/or wherein the heterologous nucleic acid sequence encodes GFPT1 (e.g., a polypeptide represented by UniProt Accession number Q06210 to a polypeptide comprising an amino acid sequence having at least 95% sequence identity thereto).
1509. The method of any one of embodiments 431 to 432 and 1374 to 1377, wherein the disease or condition is Congenital Myasthenic Syndrome and/or wherein the heterologous nucleic acid sequence encodes SCN4A (e.g., a polypeptide represented by UniProt Accession number P35499 to a polypeptide comprising an amino acid sequence having at least 95% sequence identity thereto).
1510. The method of any one of embodiments 431 to 432 and 1374 to 1377, wherein the disease or condition is Congenital Myasthenic Syndrome and/or wherein the heterologous nucleic acid sequence encodes ALG2 (e.g., a polypeptide represented by UniProt Accession number Q9H553 to a polypeptide comprising an amino acid sequence having at least 95% sequence identity thereto).
1511. The method of any one of embodiments 431 to 432 and 1374 to 1377, wherein the disease or condition is Congenital Myasthenic Syndrome and/or wherein the heterologous nucleic acid sequence encodes ALG14 (e.g., a polypeptide represented by UniProt Accession number Q96F25 to a polypeptide comprising an amino acid sequence having at least 95% sequence identity thereto).
1512. The method of any one of embodiments 431 to 432 and 1374 to 1377, wherein the disease or condition is Congenital Myasthenic Syndrome and/or wherein the heterologous nucleic acid sequence encodes DPAGT1 (e.g., a polypeptide represented by UniProt Accession number Q9H3H5 to a polypeptide comprising an amino acid sequence having at least 95% sequence identity thereto).
1513. The method of any one of embodiments 431 to 432 and 1374 to 1377, wherein the disease or condition is Congenital Myasthenic Syndrome and/or wherein the heterologous nucleic acid sequence encodes CHRNE (e.g., a polypeptide represented by UniProt Accession number Q04844 to a polypeptide comprising an amino acid sequence having at least 95% sequence identity thereto).
1514. The method of any one of embodiments 431 to 432 and 1374 to 1377, wherein the disease or condition is Congenital Myasthenic Syndrome and/or wherein the heterologous nucleic acid sequence encodes CHRNA1 (e.g., a polypeptide represented by UniProt Accession number P02708 to a polypeptide comprising an amino acid sequence having at least 95% sequence identity thereto).
1515. The method of any one of embodiments 431 to 432 and 1374 to 1377, wherein the disease or condition is Congenital Myasthenic Syndrome and/or wherein the heterologous nucleic acid sequence encodes DOK7 (e.g., a polypeptide represented by UniProt Accession number Q18PE1 to a polypeptide comprising an amino acid sequence having at least 95% sequence identity thereto).
1516. The method of any one of embodiments 431 to 432 and 1374 to 1377, wherein the disease or condition is Congenital Myasthenic Syndrome and/or wherein the heterologous nucleic acid sequence encodes CHAT (e.g., a polypeptide represented by UniProt Accession number P28329 to a polypeptide comprising an amino acid sequence having at least 95% sequence identity thereto).
1517. The method of any one of embodiments 431 to 432 and 1374 to 1377, wherein the disease or condition is Congenital Myopathy and/or wherein the heterologous nucleic acid sequence encodes ACTA1 (e.g., a polypeptide represented by UniProt Accession number P68133 to a polypeptide comprising an amino acid sequence having at least 95% sequence identity thereto).
1518. The method of any one of embodiments 431 to 432 and 1374 to 1377, wherein the disease or condition is Congenital Myopathy and/or wherein the heterologous nucleic acid sequence encodes STAC3 (e.g., a polypeptide represented by UniProt Accession number Q96MF2 to a polypeptide comprising an amino acid sequence having at least 95% sequence identity thereto).
1519. The method of any one of embodiments 431 to 432 and 1374 to 1377, wherein the disease or condition is Congenital Myopathy and/or wherein the heterologous nucleic acid sequence encodes TPM3 (e.g., a polypeptide represented by UniProt Accession number P06753 to a polypeptide comprising an amino acid sequence having at least 95% sequence identity thereto).
1520. The method of any one of embodiments 431 to 432 and 1374 to 1377, wherein the disease or condition is Congenital Myotonic Dystrophy and/or wherein the heterologous nucleic acid sequence encodes DMPK (e.g., a polypeptide represented by UniProt Accession number Q09013 to a polypeptide comprising an amino acid sequence having at least 95% sequence identity thereto).
1521. The method of any one of embodiments 431 to 432 and 1374 to 1377, wherein the disease or condition is Ctoi Disease (Debrancher Enzyme Deficiency to Ftobes Disease) and/or wherein the heterologous nucleic acid sequence encodes AGL (e.g., a polypeptide represented by UniProt Accession number P35573 to a polypeptide comprising an amino acid sequence having at least 95% sequence identity thereto).
1522. The method of any one of embodiments 431 to 432 and 1374 to 1377, wherein the disease or condition is Danon disease and/or wherein the heterologous nucleic acid sequence encodes LAMP2 (e.g., a polypeptide represented by UniProt Accession number P13473 to a polypeptide comprising an amino acid sequence having at least 95% sequence identity thereto).
1523. The method of any one of embodiments 431 to 432 and 1374 to 1377, wherein the disease or condition is Dejerine-Sottas Disease and/or wherein the heterologous nucleic acid sequence encodes MPZ (e.g., a polypeptide represented by UniProt Accession number P25189 to a polypeptide comprising an amino acid sequence having at least 95% sequence identity thereto).
1524. The method of any one of embodiments 431 to 432 and 1374 to 1377, wherein the disease or condition is Dejerine-Sottas Disease and/or wherein the heterologous nucleic acid sequence encodes EGR2 (e.g., a polypeptide represented by UniProt Accession number P11161 to a polypeptide comprising an amino acid sequence having at least 95% sequence identity thereto).
1525. The method of any one of embodiments 431 to 432 and 1374 to 1377, wherein the disease or condition is Dejerine-Sottas Disease and/or wherein the heterologous nucleic acid sequence encodes PMP22 (e.g., a polypeptide represented by UniProt Accession number Q01453 to a polypeptide comprising an amino acid sequence having at least 95% sequence identity thereto).
1526. The method of any one of embodiments 431 to 432 and 1374 to 1377, wherein the disease or condition is Dejerine-Sottas Disease and/or wherein the heterologous nucleic acid sequence encodes PRX (e.g., a polypeptide represented by UniProt Accession number Q9BXM0 to a polypeptide comprising an amino acid sequence having at least 95% sequence identity thereto).
1527. The method of any one of embodiments 431 to 432 and 1374 to 1377, wherein the disease or condition is Distal Muscular Dystrophy, Welander and/or wherein the heterologous nucleic acid sequence encodes TIA1 (e.g., a polypeptide represented by UniProt Accession number P31483 to a polypeptide comprising an amino acid sequence having at least 95% sequence identity thereto).
1528. The method of any one of embodiments 431 to 432 and 1374 to 1377, wherein the disease or condition is Distal Muscular Dystrophy, Miyoshi and/or wherein the heterologous nucleic acid sequence encodes DYSF (e.g., a polypeptide represented by UniProt Accession number O75923 to a polypeptide comprising an amino acid sequence having at least 95% sequence identity thereto).
1529. The method of any one of embodiments 431 to 432 and 1374 to 1377, wherein the disease or condition is Distal myopathy with anterito tibial onset and/or wherein the heterologous nucleic acid sequence encodes DYSF (e.g., a polypeptide represented by UniProt Accession number O75923 to a polypeptide comprising an amino acid sequence having at least 95% sequence identity thereto).
1530. The method of any one of embodiments 431 to 432 and 1374 to 1377, wherein the disease or condition is Duchenne Muscular Dystrophy and/or wherein the heterologous nucleic acid sequence encodes Dystrophin (DMD) (e.g., a polypeptide represented by UniProt Accession number P11532 to a polypeptide comprising an amino acid sequence having at least 95% sequence identity thereto).
1531. The method of any one of embodiments 431 to 432 and 1374 to 1377, wherein the disease or condition is Duchenne Muscular Dystrophy and/or wherein the heterologous nucleic acid sequence encodes GALGT2, B4GALNT2 (e.g., a polypeptide represented by UniProt Accession number Q8NHY0 to a polypeptide comprising an amino acid sequence having at least 95% sequence identity thereto).
1532. The method of any one of embodiments 431 to 432 and 1374 to 1377, wherein the disease or condition is Dysferlinopathy and/or wherein the heterologous nucleic acid sequence encodes Dysferlin (DYSF) (e.g., a polypeptide represented by UniProt Accession number O75923 to a polypeptide comprising an amino acid sequence having at least 95% sequence identity thereto).
1533. The method of any one of embodiments 431 to 432 and 1374 to 1377, wherein the disease or condition is Emery-Dreifuss Muscular Dystrophy and/or wherein the heterologous nucleic acid sequence encodes EMD (e.g., a polypeptide represented by UniProt Accession number P50402 to a polypeptide comprising an amino acid sequence having at least 95% sequence identity thereto).
1534. The method of any one of embodiments 431 to 432 and 1374 to 1377, wherein the disease or condition is Emery-Dreifuss Muscular Dystrophy and/or wherein the heterologous nucleic acid sequence encodes SYNE1 (e.g., a polypeptide represented by UniProt Accession number Q8NF91 to a polypeptide comprising an amino acid sequence having at least 95% sequence identity thereto).
1535. The method of any one of embodiments 431 to 432 and 1374 to 1377, wherein the disease or condition is Emery-Dreifuss Muscular Dystrophy and/or wherein the heterologous nucleic acid sequence encodes SYNE2 (e.g., a polypeptide represented by UniProt Accession number Q8WXH0 to a polypeptide comprising an amino acid sequence having at least 95% sequence identity thereto).
1536. The method of any one of embodiments 431 to 432 and 1374 to 1377, wherein the disease or condition is Emery-Dreifuss Muscular Dystrophy and/or wherein the heterologous nucleic acid sequence encodes TMEM43 (e.g., a polypeptide represented by UniProt Accession number Q9BTV4 to a polypeptide comprising an amino acid sequence having at least 95% sequence identity thereto).
1537. The method of any one of embodiments 431 to 432 and 1374 to 1377, wherein the disease or condition is Emery-Dreifuss Muscular Dystrophy and/or wherein the heterologous nucleic acid sequence encodes LMNA (e.g., a polypeptide represented by UniProt Accession number P02545 to a polypeptide comprising an amino acid sequence having at least 95% sequence identity thereto).
1538. The method of any one of embodiments 431 to 432 and 1374 to 1377, wherein the disease or condition is Eulenberg Disease (Paramyotonia Congenita) and/or wherein the heterologous nucleic acid sequence encodes SCN4A (e.g., a polypeptide represented by UniProt Accession number P35499 to a polypeptide comprising an amino acid sequence having at least 95% sequence identity thereto).
1539. The method of any one of embodiments 431 to 432 and 1374 to 1377, wherein the disease or condition is Facioscapulohumeral Muscular Dystrophy and/or wherein the heterologous nucleic acid sequence encodes SMCHD1 (e.g., a polypeptide represented by UniProt Accession number A6NHR9 to a polypeptide comprising an amino acid sequence having at least 95% sequence identity thereto).
1540. The method of any one of embodiments 431 to 432 and 1374 to 1377, wherein the disease or condition is Facioscapulohumeral Muscular Dystrophy and/or wherein the heterologous nucleic acid sequence encodes LRIF1 (e.g., a polypeptide represented by UniProt Accession number Q5T3J3 to a polypeptide comprising an amino acid sequence having at least 95% sequence identity thereto).
1541. The method of any one of embodiments 431 to 432 and 1374 to 1377, wherein the disease or condition is Friedreich's ataxia and/or wherein the heterologous nucleic acid sequence encodes Frataxin (FXN) (e.g., a polypeptide represented by UniProt Accession number Q16595 to a polypeptide comprising an amino acid sequence having at least 95% sequence identity thereto).
1542. The method of any one of embodiments 431 to 432 and 1374 to 1377, wherein the disease or condition is Fukuyama Congenital Muscular Dystrophy and/or wherein the heterologous nucleic acid sequence encodes FKTN (e.g., a polypeptide represented by UniProt Accession number 075072 to a polypeptide comprising an amino acid sequence having at least 95% sequence identity thereto).
1543. The method of any one of embodiments 431 to 432 and 1374 to 1377, wherein the disease or condition is Glycogen sttoage disease II (Pompe disease or Acid Maltase Deficiency) and/or wherein the heterologous nucleic acid sequence encodes GAA (e.g., a polypeptide represented by UniProt Accession number P10253 to a polypeptide comprising an amino acid sequence having at least 95% sequence identity thereto).
1544. The method of any one of embodiments 431 to 432 and 1374 to 1377, wherein the disease or condition is Glycogenosis Type 10 (Glycogen sttoage disease X) and/or wherein the heterologous nucleic acid sequence encodes PGAM2 (e.g., a polypeptide represented by UniProt Accession number P15259 to a polypeptide comprising an amino acid sequence having at least 95% sequence identity thereto).
1545. The method of any one of embodiments 431 to 432 and 1374 to 1377, wherein the disease or condition is Glycogenosis Type 11 (Glycogen sttoage disease XI) and/or wherein the heterologous nucleic acid sequence encodes LDHA (e.g., a polypeptide represented by UniProt Accession number P00338 to a polypeptide comprising an amino acid sequence having at least 95% sequence identity thereto).
1546. The method of any one of embodiments 431 to 432 and 1374 to 1377, wherein the disease or condition is Glycogenosis Type 2 (Glycogen sttoage disease II to Pompe Disease or Acid maltase deficiency) and/or wherein the heterologous nucleic acid sequence encodes GAA (e.g., a polypeptide represented by UniProt Accession number P10253 to a polypeptide comprising an amino acid sequence having at least 95% sequence identity thereto).
1547. The method of any one of embodiments 431 to 432 and 1374 to 1377, wherein the disease or condition is Glycogenosis Type 3 (Glycogen sttoage disease Ill) and/or wherein the heterologous nucleic acid sequence encodes AGL (e.g., a polypeptide represented by UniProt Accession number P35573 to a polypeptide comprising an amino acid sequence having at least 95% sequence identity thereto).
1548. The method of any one of embodiments 431 to 432 and 1374 to 1377, wherein the disease or condition is Glycogenosis Type 5 (Glycogen sttoage disease V to McArdle Disease or Myophosphtoylase Deficiency) and/or wherein the heterologous nucleic acid sequence encodes PYGM (e.g., a polypeptide represented by UniProt Accession number P11217 to a polypeptide comprising an amino acid sequence having at least 95% sequence identity thereto).
1549. The method of any one of embodiments 431 to 432 and 1374 to 1377, wherein the disease or condition is Glycogenosis Type 7 (Glycogen sttoage disease VII to Phosphofructokinase Deficiency to Tarui Disease) and/or wherein the heterologous nucleic acid sequence encodes PFKM (e.g., a polypeptide represented by UniProt Accession number P08237 to a polypeptide comprising an amino acid sequence having at least 95% sequence identity thereto).
1550. The method of any one of embodiments 431 to 432 and 1374 to 1377, wherein the disease or condition is Glycogenosis Type 9 (Glycogen sttoage disease IX) and/or wherein the heterologous nucleic acid sequence encodes PHKB (e.g., a polypeptide represented by UniProt Accession number Q93100 to a polypeptide comprising an amino acid sequence having at least 95% sequence identity thereto).
1551. The method of any one of embodiments 431 to 432 and 1374 to 1377, wherein the disease or condition is Glycogenosis Type 9 (Glycogen sttoage disease IX) and/or wherein the heterologous nucleic acid sequence encodes PHKA2 (e.g., a polypeptide represented by UniProt Accession number P46019 to a polypeptide comprising an amino acid sequence having at least 95% sequence identity thereto).
1552. The method of any one of embodiments 431 to 432 and 1374 to 1377, wherein the disease or condition is Hereditary Inclusion-Body Myositis and/or wherein the heterologous nucleic acid sequence encodes GNE (e.g., a polypeptide represented by UniProt Accession number Q9Y223 to a polypeptide comprising an amino acid sequence having at least 95% sequence identity thereto).
1553. The method of any one of embodiments 431 to 432 and 1374 to 1377, wherein the disease or condition is Integrin-Deficient Congenital Muscular Dystrophy and/or wherein the heterologous nucleic acid sequence encodes ITGA7 (e.g., a polypeptide represented by UniProt Accession number Q13683 to a polypeptide comprising an amino acid sequence having at least 95% sequence identity thereto).
1554. The method of any one of embodiments 431 to 432 and 1374 to 1377, wherein the disease or condition is Kennedy Disease (Spinal-Bulbar Muscular Atrophy) and/or wherein the heterologous nucleic acid sequence encodes Androgen receptto (AR) (e.g., a polypeptide represented by UniProt Accession number P10275 to a polypeptide comprising an amino acid sequence having at least 95% sequence identity thereto).
1555. The method of any one of embodiments 431 to 432 and 1374 to 1377, wherein the disease or condition is Kugelberg-Welander Disease and/or wherein the heterologous nucleic acid sequence encodes SMN1 (e.g., a polypeptide represented by UniProt Accession number Q16637 to a polypeptide comprising an amino acid sequence having at least 95% sequence identity thereto).
1556. The method of any one of embodiments 431 to 432 and 1374 to 1377, wherein the disease or condition is Lactate dehydrogenase A deficiency and/or wherein the heterologous nucleic acid sequence encodes LDHA (e.g., a polypeptide represented by UniProt Accession number P00338 to a polypeptide comprising an amino acid sequence having at least 95% sequence identity thereto).
1557. The method of any one of embodiments 431 to 432 and 1374 to 1377, wherein the disease or condition is Lactate Dehydrogenase B Deficiency and/or wherein the heterologous nucleic acid sequence encodes LDHB (e.g., a polypeptide represented by UniProt Accession number P07195 to a polypeptide comprising an amino acid sequence having at least 95% sequence identity thereto).
1558. The method of any one of embodiments 431 to 432 and 1374 to 1377, wherein the disease or condition is Lambert-Eaton Myasthenic Syndrome and/or wherein the heterologous nucleic acid sequence encodes CACNB2 (e.g., a polypeptide represented by UniProt Accession number Q08289 to a polypeptide comprising an amino acid sequence having at least 95% sequence identity thereto).
1559. The method of any one of embodiments 431 to 432 and 1374 to 1377, wherein the disease or condition is Laing Distal Myopathy and/or wherein the heterologous nucleic acid sequence encodes MYH7 (e.g., a polypeptide represented by UniProt Accession number A7E2Y1 to a polypeptide comprising an amino acid sequence having at least 95% sequence identity thereto).
1560. The method of any one of embodiments 431 to 432 and 1374 to 1377, wherein the disease or condition is Limb Girdle Muscular Dystrophy Type 2C (LGMD-2C) and/or wherein the heterologous nucleic acid sequence encodes Gamma-sarcoglycan (e.g., a polypeptide represented by UniProt Accession number Q13326 to a polypeptide comprising an amino acid sequence having at least 95% sequence identity thereto).
1561. The method of any one of embodiments 431 to 432 and 1374 to 1377, wherein the disease or condition is Limb Girdle Muscular Dystrophy Type 2D (LGMD-2D) and/or wherein the heterologous nucleic acid sequence encodes Alpha-sarcoglycan (e.g., a polypeptide represented by UniProt Accession number Q16586 to a polypeptide comprising an amino acid sequence having at least 95% sequence identity thereto).
1562. The method of any one of embodiments 431 to 432 and 1374 to 1377, wherein the disease or condition is Limb Girdle Muscular Dystrophy Type2E (LGMD-2E) and/or wherein the heterologous nucleic acid sequence encodes Beta-sarcoglycan (e.g., a polypeptide represented by UniProt Accession number Q16585 to a polypeptide comprising an amino acid sequence having at least 95% sequence identity thereto).
1563. The method of any one of embodiments 431 to 432 and 1374 to 1377, wherein the disease or condition is Limb Girdle Muscular Dystrophy Type 2F (LGMD-2F) and/or wherein the heterologous nucleic acid sequence encodes Delta-sarcoglycan (e.g., a polypeptide represented by UniProt Accession number Q92629 to a polypeptide comprising an amino acid sequence having at least 95% sequence identity thereto).
1564. The method of any one of embodiments 431 to 432 and 1374 to 1377, wherein the disease or condition is Merosin-Deficient Congenital Muscular Dystrophy and/or wherein the heterologous nucleic acid sequence encodes LAMA2 (e.g., a polypeptide represented by UniProt Accession number P24043 to a polypeptide comprising an amino acid sequence having at least 95% sequence identity thereto).
1565. The method of any one of embodiments 431 to 432 and 1374 to 1377, wherein the disease or condition is Muscle-Eye-Brain Disease and/or wherein the heterologous nucleic acid sequence encodes POMGNT1 (e.g., a polypeptide represented by UniProt Accession number Q8WZA1 to a polypeptide comprising an amino acid sequence having at least 95% sequence identity thereto).
1566. The method of any one of embodiments 431 to 432 and 1374 to 1377, wherein the disease or condition is Mitochondrial Myopathy and/or wherein the heterologous nucleic acid sequence encodes CHCHD10 (e.g., a polypeptide represented by UniProt Accession number Q8WYQ3 to a polypeptide comprising an amino acid sequence having at least 95% sequence identity thereto).
1567. The method of any one of embodiments 431 to 432 and 1374 to 1377, wherein the disease or condition is Miyoshi myopathy and/or wherein the heterologous nucleic acid sequence encodes DYSF (e.g., a polypeptide represented by UniProt Accession number O75923 to a polypeptide comprising an amino acid sequence having at least 95% sequence identity thereto).
1568. The method of any one of embodiments 431 to 432 and 1374 to 1377, wherein the disease or condition is Myoadenylate Deaminase Deficiency and/or wherein the heterologous nucleic acid sequence encodes AMPD1 (e.g., a polypeptide represented by UniProt Accession number P23109 to a polypeptide comprising an amino acid sequence having at least 95% sequence identity thereto).
1569. The method of any one of embodiments 431 to 432 and 1374 to 1377, wherein the disease or condition is Myofibrillar Myopathy 1 and/or wherein the heterologous nucleic acid sequence encodes DES (e.g., a polypeptide represented by UniProt Accession number P17661 to a polypeptide comprising an amino acid sequence having at least 95% sequence identity thereto).
1570. The method of any one of embodiments 431 to 432 and 1374 to 1377, wherein the disease or condition is Myofibrillar Myopathy 2 and/or wherein the heterologous nucleic acid sequence encodes CRYAB (e.g., a polypeptide represented by UniProt Accession number P02511 to a polypeptide comprising an amino acid sequence having at least 95% sequence identity thereto).
1571. The method of any one of embodiments 431 to 432 and 1374 to 1377, wherein the disease or condition is Myofibrillar Myopathy 3 and/or wherein the heterologous nucleic acid sequence encodes MYOT (e.g., a polypeptide represented by UniProt Accession number Q9UBF9 to a polypeptide comprising an amino acid sequence having at least 95% sequence identity thereto).
1572. The method of any one of embodiments 431 to 432 and 1374 to 1377, wherein the disease or condition is Myofibrillar Myopathy 4 (ZASP related myopathy) and/or wherein the heterologous nucleic acid sequence encodes LDB3, ZASP (e.g., a polypeptide represented by UniProt Accession number 075112 to a polypeptide comprising an amino acid sequence having at least 95% sequence identity thereto).
1573. The method of any one of embodiments 431 to 432 and 1374 to 1377, wherein the disease or condition is Myofibrillar Myopathy 5 and/or wherein the heterologous nucleic acid sequence encodes FLNC (e.g., a polypeptide represented by UniProt Accession number Q14315 to a polypeptide comprising an amino acid sequence having at least 95% sequence identity thereto).
1574. The method of any one of embodiments 431 to 432 and 1374 to 1377, wherein the disease or condition is Myofibrillar Myopathy 6 and/or wherein the heterologous nucleic acid sequence encodes BAG3 (e.g., a polypeptide represented by UniProt Accession number O95817 to a polypeptide comprising an amino acid sequence having at least 95% sequence identity thereto).
1575. The method of any one of embodiments 431 to 432 and 1374 to 1377, wherein the disease or condition is Myofibrillar Myopathy 7 and/or wherein the heterologous nucleic acid sequence encodes KY (e.g., a polypeptide represented by UniProt Accession number Q8NBH2 to a polypeptide comprising an amino acid sequence having at least 95% sequence identity thereto).
1576. The method of any one of embodiments 431 to 432 and 1374 to 1377, wherein the disease or condition is Myofibrillar Myopathy 8 and/or wherein the heterologous nucleic acid sequence encodes PYROXD1 (e.g., a polypeptide represented by UniProt Accession number Q8WU10 to a polypeptide comprising an amino acid sequence having at least 95% sequence identity thereto).
1577. The method of any one of embodiments 431 to 432 and 1374 to 1377, wherein the disease or condition is Myofibrillar Myopathy 9 and/or wherein the heterologous nucleic acid sequence encodes TTN (e.g., a polypeptide represented by UniProt Accession number Q8WZ42 to a polypeptide comprising an amino acid sequence having at least 95% sequence identity thereto).
1578. The method of any one of embodiments 431 to 432 and 1374 to 1377, wherein the disease or condition is Myofibrillar Myopathy 10 and/or wherein the heterologous nucleic acid sequence encodes SVIL (e.g., a polypeptide represented by UniProt Accession number O95425 to a polypeptide comprising an amino acid sequence having at least 95% sequence identity thereto).
1579. The method of any one of embodiments 431 to 432 and 1374 to 1377, wherein the disease or condition is Myofibrillar Myopathy 11 and/or wherein the heterologous nucleic acid sequence encodes UNC45B (e.g., a polypeptide represented by UniProt Accession number Q8IWX7 to a polypeptide comprising an amino acid sequence having at least 95% sequence identity thereto).
1580. The method of any one of embodiments 431 to 432 and 1374 to 1377, wherein the disease or condition is Myofibrillar Myopathy 12 and/or wherein the heterologous nucleic acid sequence encodes MYL2 (e.g., a polypeptide represented by UniProt Accession number Q99972 to a polypeptide comprising an amino acid sequence having at least 95% sequence identity thereto).
1581. The method of any one of embodiments 431 to 432 and 1374 to 1377, wherein the disease or condition is Myotonic dystrophy Type 1 (Steinert Disease) and/or wherein the heterologous nucleic acid sequence encodes Myotonin-protein kinase (DMPK) (e.g., a polypeptide represented by UniProt Accession number Q09013 to a polypeptide comprising an amino acid sequence having at least 95% sequence identity thereto).
1582. The method of any one of embodiments 431 to 432 and 1374 to 1377, wherein the disease or condition is Myotonic dystrophy Type 2 and/or wherein the heterologous nucleic acid sequence encodes CNBP (e.g., a polypeptide represented by UniProt Accession number P62633 to a polypeptide comprising an amino acid sequence having at least 95% sequence identity thereto).
1583. The method of any one of embodiments 431 to 432 and 1374 to 1377, wherein the disease or condition is Myotubular Myopathy and/or wherein the heterologous nucleic acid sequence encodes MTM1 (e.g., a polypeptide represented by UniProt Accession number Q13496 to a polypeptide comprising an amino acid sequence having at least 95% sequence identity thereto).
1584. The method of any one of embodiments 431 to 432 and 1374 to 1377, wherein the disease or condition is Nemaline Myopathy 1 and/or wherein the heterologous nucleic acid sequence encodes TPM3 (e.g., a polypeptide represented by UniProt Accession number P06753 to a polypeptide comprising an amino acid sequence having at least 95% sequence identity thereto).
1585. The method of any one of embodiments 431 to 432 and 1374 to 1377, wherein the disease or condition is Nemaline Myopathy 2 and/or wherein the heterologous nucleic acid sequence encodes NEB (e.g., a polypeptide represented by UniProt Accession number P20929 to a polypeptide comprising an amino acid sequence having at least 95% sequence identity thereto).
1586. The method of any one of embodiments 431 to 432 and 1374 to 1377, wherein the disease or condition is Nemaline Myopathy 5A, 5B, 5C and/or wherein the heterologous nucleic acid sequence encodes TNNT1 (e.g., a polypeptide represented by UniProt Accession number P13805 to a polypeptide comprising an amino acid sequence having at least 95% sequence identity thereto).
1587. The method of any one of embodiments 431 to 432 and 1374 to 1377, wherein the disease or condition is Nemaline Myopathy 3 and/or wherein the heterologous nucleic acid sequence encodes ACTA1 (e.g., a polypeptide represented by UniProt Accession number P68133 to a polypeptide comprising an amino acid sequence having at least 95% sequence identity thereto).
1588. The method of any one of embodiments 431 to 432 and 1374 to 1377, wherein the disease or condition is Nemaline Myopathy 6 and/or wherein the heterologous nucleic acid sequence encodes KBTBD13 (e.g., a polypeptide represented by UniProt Accession number C9JR72 to a polypeptide comprising an amino acid sequence having at least 95% sequence identity thereto).
1589. The method of any one of embodiments 431 to 432 and 1374 to 1377, wherein the disease or condition is Nemaline Myopathy 4 and/or wherein the heterologous nucleic acid sequence encodes TPM2 (e.g., a polypeptide represented by UniProt Accession number P07951 to a polypeptide comprising an amino acid sequence having at least 95% sequence identity thereto).
1590. The method of any one of embodiments 431 to 432 and 1374 to 1377, wherein the disease or condition is Nemaline Myopathy 7 and/or wherein the heterologous nucleic acid sequence encodes CFL2 (e.g., a polypeptide represented by UniProt Accession number Q9Y281 to a polypeptide comprising an amino acid sequence having at least 95% sequence identity thereto).
1591. The method of any one of embodiments 431 to 432 and 1374 to 1377, wherein the disease or condition is Nemaline Myopathy 8 and/or wherein the heterologous nucleic acid sequence encodes KLHL40 (e.g., a polypeptide represented by UniProt Accession number Q2TBA0 to a polypeptide comprising an amino acid sequence having at least 95% sequence identity thereto).
1592. The method of any one of embodiments 431 to 432 and 1374 to 1377, wherein the disease or condition is Nemaline Myopathy 9 and/or wherein the heterologous nucleic acid sequence encodes KLHL41 (e.g., a polypeptide represented by UniProt Accession number O60662 to a polypeptide comprising an amino acid sequence having at least 95% sequence identity thereto).
1593. The method of any one of embodiments 431 to 432 and 1374 to 1377, wherein the disease or condition is Nemaline Myopathy 10 and/or wherein the heterologous nucleic acid sequence encodes LMOD3 (e.g., a polypeptide represented by UniProt Accession number Q0VAK6 to a polypeptide comprising an amino acid sequence having at least 95% sequence identity thereto).
1594. The method of any one of embodiments 431 to 432 and 1374 to 1377, wherein the disease or condition is Nonaka Distal Myopathy and/or wherein the heterologous nucleic acid sequence encodes GNE (e.g., a polypeptide represented by UniProt Accession number Q9Y223 to a polypeptide comprising an amino acid sequence having at least 95% sequence identity thereto).
1595. The method of any one of embodiments 431 to 432 and 1374 to 1377, wherein the disease or condition is Oculopharynggeal muscular dystrophy and/or wherein the heterologous nucleic acid sequence encodes PABPN1 (e.g., a polypeptide represented by UniProt Accession number Q86U42 to a polypeptide comprising an amino acid sequence having at least 95% sequence identity thereto).
1596. The method of any one of embodiments 431 to 432 and 1374 to 1377, wherein the disease or condition is Omithine Transcarbamylase deficiency and/or wherein the heterologous nucleic acid sequence encodes OTC (e.g., a polypeptide represented by UniProt Accession number P00480 to a polypeptide comprising an amino acid sequence having at least 95% sequence identity thereto).
1597. The method of any one of embodiments 431 to 432 and 1374 to 1377, wherein the disease or condition is Paramyotonia Congenita and/or wherein the heterologous nucleic acid sequence encodes SCN4A (e.g., a polypeptide represented by UniProt Accession number P35499 to a polypeptide comprising an amino acid sequence having at least 95% sequence identity thereto).
1598. The method of any one of embodiments 431 to 432 and 1374 to 1377, wherein the disease or condition is Periodic Paralysis, hypokalemic and/or wherein the heterologous nucleic acid sequence encodes CACNA1S (e.g., a polypeptide represented by UniProt Accession number Q13698 to a polypeptide comprising an amino acid sequence having at least 95% sequence identity thereto).
1599. The method of any one of embodiments 431 to 432 and 1374 to 1377, wherein the disease or condition is Periodic Paralysis, hyperkalemic and/or wherein the heterologous nucleic acid sequence encodes SCN4A (e.g., a polypeptide represented by UniProt Accession number P35499 to a polypeptide comprising an amino acid sequence having at least 95% sequence identity thereto).
1600. The method of any one of embodiments 431 to 432 and 1374 to 1377, wherein the disease or condition is Phosphoglycerate Kinase Deficiency and/or wherein the heterologous nucleic acid sequence encodes PGK1 (e.g., a polypeptide represented by UniProt Accession number P00558 to a polypeptide comprising an amino acid sequence having at least 95% sequence identity thereto).
1601. The method of any one of embodiments 431 to 432 and 1374 to 1377, wherein the disease or condition is Polymyositis and/or wherein the heterologous nucleic acid sequence encodes PMSCL2 (e.g., a polypeptide represented by UniProt Accession number Q01780 to a polypeptide comprising an amino acid sequence having at least 95% sequence identity thereto).
1602. The method of any one of embodiments 431 to 432 and 1374 to 1377, wherein the disease or condition is Polymyositis and/or wherein the heterologous nucleic acid sequence encodes PMSCL1 (e.g., a polypeptide represented by UniProt Accession number Q06265 to a polypeptide comprising an amino acid sequence having at least 95% sequence identity thereto).
1603. The method of any one of embodiments 431 to 432 and 1374 to 1377, wherein the disease or condition is Progressive External Ophthalmoplegia and/or wherein the heterologous nucleic acid sequence encodes POLG (e.g., a polypeptide represented by UniProt Accession number P54098 to a polypeptide comprising an amino acid sequence having at least 95% sequence identity thereto).
1604. The method of any one of embodiments 431 to 432 and 1374 to 1377, wherein the disease or condition is Progressive External Ophthalmoplegia and/or wherein the heterologous nucleic acid sequence encodes POLG2 (e.g., a polypeptide represented by UniProt Accession number Q9UHN1 to a polypeptide comprising an amino acid sequence having at least 95% sequence identity thereto).
1605. The method of any one of embodiments 431 to 432 and 1374 to 1377, wherein the disease or condition is Progressive External Ophthalmoplegia and/or wherein the heterologous nucleic acid sequence encodes SLC25A4 (e.g., a polypeptide represented by UniProt Accession number P12235 to a polypeptide comprising an amino acid sequence having at least 95% sequence identity thereto).
1606. The method of any one of embodiments 431 to 432 and 1374 to 1377, wherein the disease or condition is Progressive External Ophthalmoplegia and/or wherein the heterologous nucleic acid sequence encodes TWNK (e.g., a polypeptide represented by UniProt Accession number Q96RR1 to a polypeptide comprising an amino acid sequence having at least 95% sequence identity thereto).
1607. The method of any one of embodiments 431 to 432 and 1374 to 1377, wherein the disease or condition is Spinal Muscular Atrophy type 3—Kugelberg-Welander Disease and/or wherein the heterologous nucleic acid sequence encodes Survival motto neuron protein (SMN), SMN1 (e.g., a polypeptide represented by UniProt Accession number Q16637 to a polypeptide comprising an amino acid sequence having at least 95% sequence identity thereto).
1608. The method of any one of embodiments 431 to 432 and 1374 to 1377, wherein the disease or condition is Thomsen Disease (Myotonia Congenita (autosomal dominant)) and/or wherein the heterologous nucleic acid sequence encodes CLCN1 (e.g., a polypeptide represented by UniProt Accession number P35523 to a polypeptide comprising an amino acid sequence having at least 95% sequence identity thereto).
1609. The method of any one of embodiments 431 to 432 and 1374 to 1377, wherein the disease or condition is Walker-Warburg Syndrome and/or wherein the heterologous nucleic acid sequence encodes POMT1 (e.g., a polypeptide represented by UniProt Accession number Q9Y6A1 to a polypeptide comprising an amino acid sequence having at least 95% sequence identity thereto).
1610. The method of any one of embodiments 431 to 432 and 1374 to 1377, wherein the disease or condition is X-linked myotubular myopathy and/or wherein the heterologous nucleic acid sequence encodes Myotubularin (MTM1) (e.g., a polypeptide represented by UniProt Accession number Q13496 to a polypeptide comprising an amino acid sequence having at least 95% sequence identity thereto).
1611. The method of any one of embodiments 431 to 432 and 1374 to 1377, wherein the disease or condition is Werdnig-Hoffmann Disease—Spinal Muscular Atrophy type 1 and/or wherein the heterologous nucleic acid sequence encodes SMN1 (e.g., a polypeptide represented by UniProt Accession number Q16637 to a polypeptide comprising an amino acid sequence having at least 95% sequence identity thereto).
1612. The method of any one of embodiments 431 to 432 and 1374 to 1377, wherein the disease or condition is Hutchinson-Gilftod progeria syndrome (HGPS) and/or wherein the heterologous nucleic acid sequence encodes an antisense oligonucleotide targeting human lamin A (LMNA) (e.g., a polypeptide represented by UniProt Accession number P02545 to a polypeptide comprising an amino acid sequence having at least 95% sequence identity thereto).
1613. The method of any one of embodiments 431 to 432 and 1374 to 1377, wherein the disease or condition is Spinal Muscular Atrophy (SMA) and/or wherein the heterologous nucleic acid sequence encodes an antisense oligonucleotide targeting human Survival Motto Neuron (SMN) (e.g., a polypeptide represented by UniProt Accession number Q16637 to a polypeptide comprising an amino acid sequence having at least 95% sequence identity thereto).
1614. The method of any one of embodiments 431 to 432 and 1374 to 1377, wherein the disease or condition is Pompe disease and/or wherein the heterologous nucleic acid sequence encodes an antisense oligonucleotide targeting human acid alpha-glucosidase (GAA) (e.g., a polypeptide represented by UniProt Accession number P10253 to a polypeptide comprising an amino acid sequence having at least 95% sequence identity thereto).
1615. The method of any one of embodiments 431 to 432 and 1374 to 1377, wherein the disease or condition is Duchenne muscular dystrophy and/or wherein the heterologous nucleic acid sequence encodes an antisense oligonucleotide targeting human Dystrophin (DMD) (e.g., a polypeptide represented by UniProt Accession number P11532 to a polypeptide comprising an amino acid sequence having at least 95% sequence identity thereto).
1616. The method of any one of embodiments 431 to 432 and 1374 to 1377, wherein the disease or condition is Myotonic dystrophy Type 1—Steinert Disease and/or wherein the heterologous nucleic acid sequence encodes an antisense oligonucleotide targeting human Dystrophia Myotonica-Protein Kinase (DMPK) (e.g., a polypeptide represented by UniProt Accession number Q09013 to a polypeptide comprising an amino acid sequence having at least 95% sequence identity thereto).
1617. The method of any one of embodiments 431 to 432 and 1374 to 1377, wherein the disease or condition is Muscle atrophy and/or wherein the heterologous nucleic acid sequence encodes an antisense oligonucleotide targeting human Myostatin (MSTN) (e.g., a polypeptide represented by UniProt Accession number 014793 to a polypeptide comprising an amino acid sequence having at least 95% sequence identity thereto).
1618. The method of any one of embodiments 431 to 432 and 1374 to 1377, wherein the disease or condition is Facioscapulohumeral Muscular Dystrophy and/or wherein the heterologous nucleic acid sequence encodes an antisense oligonucleotide targeting human Double homeobox 4 (DUX4) (e.g., a polypeptide represented by UniProt Accession number Q9UBX2 or a polypeptide comprising an amino acid sequence having at least 95% sequence identity thereto).
1619. The method of embodiment 431 or 432, wherein the disease or condition is a disease or condition identified in Section 6.7, 6.7.1, or 6.7.2 and/or wherein the heterologous nucleic acid sequence encodes a protein identified in Section 6.7, 6.7.1, or 6.7.2.
1620. The method of any one of embodiments 430 to 1619, wherein the subject is a mammal, e.g., a human.
1621. A cell, cell-free system, or other translation system, comprising the capsid polypeptide of any one of embodiments 1 to 326, the nucleic acid molecule of any one of embodiments 327 to 397, or virus particle of any one of embodiments 398 to 429.
1622. A method of making a virus (e.g., a dependoparvovirus particle such as an adeno-associated virus (AAV) particle), comprising:
   (a) providing a cell, cell-free system, or other translation system, comprising the nucleic acid of any one of embodiments 327 to 397; and
   (b) cultivating the cell, cell-free system, or other translation system, under conditions suitable for the production of the virus particle, thereby making the virus particle.
1623. The method of embodiment 1622, wherein the cell, cell-free system, or other translation system comprises a second nucleic acid molecule and at least a portion of said second nucleic acid molecule is packaged in the dependoparvovirus particle.
1624. The method of embodiment 1623, wherein the second nucleic acid comprises a payload, e.g., a heterologous nucleic acid sequence encoding a therapeutic product, e.g., as described herein.
1625. The method of any one of embodiments 1622 to 1624, wherein the nucleic acid molecule of any one of embodiments 327 to 397 mediates the production of a virus particle which does not include said nucleic acid of any one of embodiments 327 to 397 or fragment thereof.
1626. The method of any one of embodiments 1622 to 1625, wherein the nucleic acid molecule of any one of embodiments 327 to 397 mediates the production of a virus particle at a level at least 40%, at least 50%, at least 60%, at least 70%, or at least 80% of the production level mediated by a nucleic acid molecule comprising SEQ ID NO:8 in an otherwise similar production system.
1627. The method of any one of embodiments 1622 to 1625, wherein the nucleic acid molecule of any one of embodiments 327 to 397 mediates the production of a virus particle at a level similar, at least 10% greater, or at least 20% greater than the production level mediated by a nucleic acid comprising SEQ ID NO:8 in an otherwise similar production system.
1628. In a method of making a virus (e.g., a dependoparvovirus particle such as an adeno-associated virus (AAV) particle) that comprises providing a cell, cell-free system, or other translation system comprising a nucleic acid molecule encoding a capsid polypeptide; and cultivating the cell, cell-free system, or other translation system under conditions suitable for the production of the virus particle, the improvement comprising utilizing a nucleic acid molecule encoding a capsid polypeptide comprising:
   (a) a histidine at a position corresponding to R550 of the VP1 capsid polypeptide of SEQ ID NO:7;
   (b) an asparagine at a position corresponding to D551 of the VP1 capsid polypeptide of SEQ ID NO:7;
   (c) an alanine at a position corresponding to S576 of the VP1 capsid polypeptide of SEQ ID NO:7;
   (d) an isoleucine at a position corresponding to Q579 of the VP1 capsid polypeptide of SEQ ID NO:7;
   (e) a lysine at a position corresponding to H584 of the VP1 capsid polypeptide of SEQ ID NO:7;
   (f) an asparagine at a position corresponding to S586 of the VP1 capsid polypeptide of SEQ ID NO:7;
   (g) a histidine or serine at a position corresponding to A587 of the VP1 capsid polypeptide of SEQ ID NO:7;
   (h) a tyrosine, a threonine, or asparagine at a position corresponding to Q588 of the VP1 capsid polypeptide of SEQ ID NO:7;
   (i) a glutamine at a position corresponding to A589 of the VP1 capsid polypeptide of SEQ ID NO:7;
   (j) an asparagine at a position corresponding to Q592 of the VP1 capsid polypeptide of SEQ ID NO:7; or
   (k) any combination of two, three, four, five, six, seven, eight, nine, or all ten of (a), (b), (c), (d), (e), (f), (g), (h), (i), and (j), optionally wherein the combination is a combination described in any one of embodiments 3 to 67.
1629. The method of embodiment 1628, wherein the cell, cell-free system, or other translation system comprises a second nucleic acid molecule and at least a portion of said second nucleic acid molecule is packaged in the dependoparvovirus particle.
1630. The method of embodiment 1629, wherein the second nucleic acid molecule comprises a payload, e.g., a heterologous nucleic acid sequence encoding a therapeutic product, e.g., as described herein.
1631. The method of any one of embodiments 1628 to 1630, wherein the nucleic acid molecule encoding the capsid polypeptide mediates the production of a virus particle which does not include said nucleic acid molecule encoding the capsid polypeptide or fragment thereof.
1632. The method of any one of embodiments 1628 to 1631, wherein the nucleic acid molecule encoding the capsid polypeptide mediates the production of a virus particle at a level at least 40%, at least 50%, at least 60%, at least 70%, or at least 80% of the production level mediated by a nucleic acid molecule comprising SEQ ID NO:8 in an otherwise similar production system.
1633. The method of any one of embodiments 1628 to 1631, wherein the nucleic acid molecule encoding the capsid polypeptide mediates the production of a virus particle at a level similar, at least 10% greater, or at least 20% greater than the production level mediated by a nucleic acid comprising SEQ ID NO:8 in an otherwise similar production system.
1634. A composition, e.g., a pharmaceutical composition, comprising a virus particle of any one of embodiments 398 to 429, or a virus particle produced by the method of any one of embodiments 1622 to 1632, and a pharmaceutically acceptable carrier or excipient.
1635. The capsid polypeptide of any one of embodiments 1 to 326, the nucleic acid molecule of any one of embodiments 327 to 397, the virus particle of any one of embodiments 398 to 429, or the composition of embodiment 1634, for use in treating a disease or condition in a subject, optionally wherein (a) the subject is as defined in embodiment 1620 and/or (b) the disease or condition is as defined in any one of embodiments 433 to 1619.
1636. The capsid polypeptide of any one of embodiments 1 to 326, the nucleic acid molecule of any one of embodiments 327 to 397, or the virus particle of any one of embodiments 398 to 429, or the composition of embodiment 1634, for use in the manufacture of a medicament for use in treating a disease or condition in a subject, optionally wherein (a) the subject is as defined in embodiment 1620 and/or (b) the disease or condition is as defined in any one of embodiments 433 to 1619.
1637. A host cell comprising the nucleic acid of any one of embodiments 327 to 397.
1638. The host cell of embodiment 1637, wherein the host cell comprises a nucleic acid encoding a rep protein.
1639. The host cell embodiment 1637 or 1638, wherein the host cell comprises a nucleic acid comprising one or more helper sequences.
1640. The host cell of any one of embodiments 1637 to 1639, wherein the host cell comprises a nucleic acid comprising a payload, e.g., a heterologous nucleic acid sequence encoding a therapeutic product, e.g., as described herein.
1641. The host cell of any one of embodiments 1637 to 1640, which is a mammalian host cell.
1642. The host cell of embodiment 1641, which is a human host cell.
1643. The host cell of any one of embodiments 1637 to 1640, which is an insect host cell.
1644. The host cell of any one of embodiments 1637 to 1643, wherein the host cell is selected from cell types described in Section 6.6.
1645. The host cell of any one of embodiments 1637 to 1644, which is a packaging host cell.
1646. The host cell of embodiment 1645, which is configured to package a virus (a) according to any one of embodiments 398 to 429 and/or (b) useful for performing a method according to any one of embodiments 430 to 1620.
1647. The method of any one of embodiments 1622 to 1633, wherein the cell is a host cell according to any one of embodiments 1637 to 1646.

### 8. EXAMPLES

The invention is further illustrated by the following examples. The examples are provided for illustrative purposes only and are not to be construed as limiting the scope or content of the invention in any way.

Unless otherwise noted, biodistribution, transduction and/or production are presented as fold-improvement over the rates exhibited by a virus particle comprising capsid polypeptides of SEQ ID NO:7.

### 8.1. Example 1: High-throughput Library Evaluation in the Muscle, Brain and Other Tissues

### 8.1.1. Materials and Methods

### 8.1.1.1. Library Creation

Using machine learning algorithms trained on data from hundreds of thousands of capsid variants across multiple serotypes, including AAV particle production efficiency, and transduction and biodistribution across multiple tissues, a library of 2.5E⁵ capsid variants was designed with the goal of producing a capsid that would package into AAV particles, transduce skeletal muscle, heart, and central nervous system tissues after intravenous injection with high efficiency, and detarget the liver and other tissue types. The designed capsid polypeptides were cloned into plasmids to create a library of plasmids encoding the capsid variants. A library of AAV variant genomes encoding each variant's capsid and a unique capsid variant barcode identifier was cloned into one ITR plasmid backbone as described previously (Ogden et al. Science, Nov. 19, 2019, 366, (6469):1139-1143; doi: 10.1126/science.aaw2900, hereby incorporated by reference in its entirety), with expression of the capsid gene under the control of a ubiquitous CBh promoter. Each capsid polypeptide variant was included in combination with between 1-500 unique genomic identifiers (“barcodes”) to enable measurement of biological replicates for each virus comprising a unique capsid polypeptide. Each unique barcode is used in combination with a random genomic identifier (“ID tag”) to aid in quantification and to remove polypeptide sequences that contain unintended mutations. A library of AAV capsid variants, each comprising a genome encoding that variant's capsid polypeptides, was produced via transient triple transfection of adherent HEK293T. Transfections were completed at a 1:1:1/150 ratio of Helper, Rep, and ITR plasmid expressing Cap, which has been optimized with these plasmids to limit cross packaging. Cells were harvested, lysed, and purified by a sequence of steps: (1) clarification via depth and sterilizing filtration, (2) ultrafiltration and diafiltration via tangential flow filtration (TFF), (3) iodixanol gradient purification, and (4) ultrafiltration and diafiltration via TFF. The produced virus was tested for suitable sterility and endotoxin, and titer performed by ddPCR.

### 8.1.1.2. Evaluation of Library Packaging Efficiency

Data was prepared as described below. To measure each variant's packaging efficiency (or “production”), barcodes from vector genomes in the plasmid library and produced AAV library were prepared for Illumina sequencing using two rounds of PCR. Production efficiency for each of the produced AAV particle variants was normalized for its abundance in the input plasmid library, and was expressed by comparing barcode sequencing levels for each variant in the produced AAV particle vector pool to the barcode sequence levels for each variant in the input plasmid library used to create the AAV vector pool. The measurements of variant frequency in the AAV vector library also enable downstream normalization of biodistribution and transduction measurements by variant frequency in the input vector library.

### 8.1.1.3. In Vivo Evaluation of Library in Non-Human Primate

All non-human primate (NHP) experiments were conducted in accordance with institutional policies and NIH guidelines. Two female Cynomolgus Macaque primates weighing 2.5 kg and 3.0 kg were selected for the study based on negative AAV neutralizing antibody serum titer results. The animals received an intravenous injection of the AAV library at 6.24e13 vg/kg or 6.96e13 vg/kg. During the in-life period the animals were monitored according to the animal facility's SOPs, and blood was sampled at regular intervals for hematology and clinical chemistry. Animals were treated with Methylprednisolone (80 mg IM) on Day −8, Day −1, and weekly throughout the study.

The animals were sacrificed 4 weeks after the injections and tissues were collected for biodistribution and transduction analyses. The tissues collected are shown in Table 2. Samples were either flash frozen in liquid nitrogen or collected into RNAlater® (Sigma-Aldrich) and incubated overnight at 4° C., after which the RNAlater® was drained and samples were frozen at −80° C.

TABLE 2
List of tissue collected, and number of samples used
for biodistribution and transduction analysis.

| | # Samples for transduction | # Samples for |
|---|---|---|
| Tissue | analysis | biodistribution analysis |
| Skeletal muscle | 34 | 34 |
| (Biceps brachii) | | |
| Skeletal muscle | 20 | 20 |
| (Quadriceps) | | |
| Skeletal muscle | 20 | 20 |
| (Gastrocnemius) | | |
| Skeletal muscle | 20 | 20 |
| (Diaphragm) | | |
| Heart | 24 | 24 |
| Liver | 21 | 24 |
| Brain | 93 | 93 |
| Spleen | 8 | 8 |

### 8.1.1.4. Bulk Sequencing NGS Library Preparation

Cardiac muscle was dissected to isolate subregions including ventricles, papillary muscle, and atria. Brain slices were dissected to isolate regions including, but not limited to, basal ganglia, brainstem, cerebral cortex, hippocampus and thalamus. For all biodistribution and transduction analyses, total DNA was extracted from tissue samples using MagMAX DNA Multi-Sample Ultra 2.0 Kit (Thermofisher). Samples were first homogenized in the supplied lysis buffer, then incubated at 65° C. with Proteinase K before the lysates were added to a plate for automated extraction using the KingFisher Apex instrument (Thermofisher). RNA was extracted using the MagMAX™ mirVana™ Total RNA Isolation Kit (Thermofisher) according to the manufacturer's recommendations, and was further treated with DNase I-XT (New England Biolabs) to remove any vector DNA contamination in the RNA sample. Reverse transcription was done with Protoscript II Reverse Transcriptase (New England Biolabs) utilizing primers that were specific to the vector transgene and included unique molecular identifiers (UMIs). Control reactions lacking the reverse transcriptase enzyme (−RT control) were also prepared. Finally, samples were prepared for next-generation sequencing by amplifying the transgene barcode regions with primers compatible with Illumina NGS platform and sequenced with NextSeq 2000 (Illumina).

### 8.1.1.5. Bulk Tissue NGS Sample Parsing and Analysis

After sequencing, the barcode tags were extracted from reads with the expected amplicon structure, and the abundance (number of reads, number of UMIs, or number of unique combinations of barcode and ID tags) of each barcode was recorded. Analyses were restricted to the set of barcodes that were present in the input plasmid sample, as measured by a separate sequencing assay that targeted the variant regions of the input plasmid sample.

To aggregate packaging replicates, the read counts from replicate virus production samples were summed. To aggregate biodistribution samples, read counts from samples from the same tissue or organ were summed. To aggregate transduction samples, the number of transduction events (measured by the number of unique barcode and ID tag combinations detected) from samples from the same tissue or organ were summed.

Virus packaging was calculated by normalizing aggregated production replicates to input plasmid abundance. Biodistribution and transduction of tissue were calculated by normalizing aggregated biodistribution or transduction samples to input virus abundance. The output was reported as a fold change relative to the WT AAV9 as indicated.

### 8.1.2. Results

The production efficiency, biodistribution, and transduction results for AAV capsid variants V1, V2, V3, and V4 are summarized in Table 3 (production efficiency), Table 4 (biodistribution), and Table 5 (transduction).

TABLE 3
Production efficiency of AAV capsid variants relative
to production efficiency of WT AAV9 (amino acid sequence -
SEQ ID NO: 7; nucleotide sequence - SEQ ID NO: 8)
virus particles, in HEK293 cells in vitro.

| | V1 | V2 | V3 | V4 |
|---|---|---|---|---|
| production | 0.88 | 0.72 | 1.2 | 1.13 |

The production efficiency ranged between 0.72 and 1.13-fold for the AAV capsid variants V1, V2, V3, and V4, each of which showed increases in biodistribution and transduction in brain, skeletal and cardiac muscle tissues, and a decrease in biodistribution in the liver. In general, each these variants was associated with more than a 30-fold increase in brain biodistribution and more than 60-fold increase in transduction in brain tissues relative to wildtype AAV9. All four capsid variants showed more than an 8-fold increase in biodistribution and more than 13-fold increase in transduction in cardiac and aggregated skeletal muscle tissues relative to wildtype AAV9.

TABLE 4
Fold change in biodistribution of virus particles comprising the
indicated AAV capsid variant polypeptide, relative to the biodistribution
of virus particles comprising WT AAV9 (amino acid sequence -
SEQ ID NO: 7; nucleotide sequence - SEQ ID NO: 8) for the indicated
tissues. “Aggregated” in relation to a tissue is an average
across all measured areas in the specified organ.

| | Tissue (if | | | | |
|---|---|---|---|---|---|
| Organ | applicable) | V1 | V2 | V3 | V4 |
| brain | aggregated | 30.6 | 36.18 | 41.99 | 47.37 |
| cardiac muscle | — | 17.34 | 22.33 | 14.71 | 8.75 |
| liver | — | 0.25 | 0.59 | 0.46 | 0.12 |
| skeletal muscle | aggregated | 34.77 | 35.39 | 14.1 | 13.64 |
| | biceps | 40.95 | 47.85 | 9.02 | 20.3 |
| | diaphragm | 15.72 | 15.65 | 6.93 | 2.7 |
| | gastrocnemius | 36.59 | 46.24 | 25.55 | 16.03 |
| | quadriceps | 37.15 | 22.66 | 14.02 | 10.12 |
| spleen | — | 6.98 | 5.9 | 2.79 | 1.07 |

TABLE 5
Fold change in transduction of virus particles comprising the
indicated AAV capsid variant polypeptide, relative to the transduction
of virus particles comprising WT AAV9 (amino acid sequence -
SEQ ID NO: 7; nucleotide sequence - SEQ ID NO: 8) for the indicated
tissues. “Aggregated” in relation to a tissue is an average
across all measured areas in the specified organ.

| | Tissue (if | | | | |
|---|---|---|---|---|---|
| Organ | applicable) | V1 | V2 | V3 | V4 |
| brain | aggregated | 69.66 | 135.32 | 126.83 | 119.93 |
| cardiac muscle | — | 13.13 | 28.11 | 20.2 | 15.86 |
| skeletal muscle | aggregated | 51.14 | 76.27 | 16.66 | 13.99 |
| | biceps | 125.06 | 201.18 | 43.04 | 50.66 |
| | diaphragm | 24.55 | 29.58 | 6.17 | 4.33 |
| | gastrocnemius | 85.4 | 126.74 | 29.39 | 20.84 |
| | quadriceps | 64.85 | 107.29 | 23.17 | 20.24 |

### 8.2. Example 2: High-throughput AAV9 Library Evaluation in the Muscle and Other Tissues

### 8.2.1. Materials and Methods

### 8.2.1.1. Library Creation

Using machine learning algorithms trained on data from hundreds of thousands of capsid variants across multiple serotypes, including AAV particle production efficiency, and transduction and biodistribution across multiple tissues, a library of 2E5 capsid variants of wild-type AAV9 was designed with the goals of producing a capsid that would package into AAV particles, transduce skeletal muscle and heart tissues after intravenous injection with high efficiency, and detarget the liver and other tissue types. The designed capsid polypeptides were cloned into plasmids to create a library of plasmids encoding the capsid variants. A library of AAV variant genomes encoding each variant's capsid and a unique capsid variant barcode identifier was cloned into one ITR plasmid backbone as described previously (Ogden et al. Science, Nov. 19, 2019, 366, (6469):1139-1143; doi: 10.1126/science.aaw2900, hereby incorporated by reference in its entirety), with expression of the capsid gene under the control of a ubiquitous CBh promoter. Each capsid polypeptide variant was included in combination with between 1-500 unique genomic identifiers (“barcodes”) to enable measurement of biological replicates for each virus comprising a unique capsid polypeptide. Each unique barcode is used in combination with a random genomic identifier (“ID tag”) to aid in quantification and to remove polypeptide sequences that contain unintended mutations. A library of AAV capsid variants, each comprising a genome encoding that variant's capsid polypeptides, was produced via transient triple transfection of adherent HEK293T. Transfections were completed at a 1:1:1/150 ratio of Helper, Rep, and ITR plasmid expressing Cap, which has been optimized with these plasmids to limit cross packaging. Cells were harvested, lysed, and purified by a sequence of steps: (1) clarification via depth and sterilizing filtration, (2) ultrafiltration and diafiltration via tangential flow filtration (TFF), (3) iodixanol gradient purification, and (4) ultrafiltration and diafiltration via TFF. The produced virus was tested for suitable sterility and endotoxin, and titer performed by ddPCR.

### 8.2.1.2. In-vivo Evaluation of Library in Mouse

All mouse experiments were conducted in accordance with institutional policies and NIH guidelines. Three 8-10 week old C57BL6/J mice, weighing 250 received an intravenous injection (tail vein) of the AAV library at 6e13vg/kg. During the in-life period the animals were monitored according to the animal facility's SOPs.

The animals were sacrificed 4 weeks after the injections and tissues were collected for biodistribution and transduction analyses. The tissues collected are shown in Table 6A. Samples were flash frozen in liquid nitrogen.

TABLE 6A
List of mouse tissues collected, and number of samples
used for biodistribution and transduction analysis.

| | # Samples for Transduction | # Samples for |
|---|---|---|
| Tissue | Analysis | Biodistribution Analysis |
| Skeletal Muscle | 9 | 8 |
| (Quadriceps) | | |
| Skeletal Muscle | 9 | 9 |
| (Gastrocnemius) | | |
| Skeletal Muscle | 9 | 9 |
| (Diaphragm) | | |
| Skeletal Muscle | 9 | 9 |
| (Biceps) | | |
| Heart | 18 | 18 |
| Liver | 12 | 12 |
| Spleen | 9 | 9 |

### 8.2.1.3. In-vivo Evaluation of Library in NHP

All NHP experiments were conducted in accordance with institutional policies and NIH guidelines. Two cynomolgus macaques (Macaca fascicularis), one female and one male weighing 2.9 and 2.7 kg, respectively, were selected for the study based on negative AAV9 neutralizing antibody serum titer results. Prior to test article administrations, samples of blood were collected. The animals received an intravenous injection of the AAV library at 6e13 vg/kg or 5.98e13 vg/kg. During the in-life period the animals were monitored for signs of inflammation and were treated with weekly IM injections of steroids (methylprednisolone, 40 mg) according to the animal facility's SOPs and recommendations from the veterinarian. Serum samples were collected 4 hours, 2 days, 5 days, and weekly after the injections. The animals were sacrificed 4 weeks after the injections and tissues were collected for biodistribution and transduction analyses. The tissues collected are shown in Table 6B. Peripheral tissue samples were collected into RNAlatet® (Sigma-Aldrich) and incubated overnight at 4° C., after which the RNAlatet® was drained and samples were frozen at −80° C. The entire brain was sliced into 4 mm coronal slabs using a chilled species-specific brain matrix. Each slab was bisected into left and right hemispheres along the sagittal midline. The slabs from the right hemisphere were immersed in RNAlater®, drained and frozen. The left hemisphere slabs were sub-dissected for sub regions of the brain, placed into cryo tubes and immediately frozen on dry-ice.

TABLE 6B
List of tissue collected, and number of samples used
for biodistribution and transduction analysis.

| | # Samples for Transduction | # Samples for |
|---|---|---|
| Tissue | Analysis | Biodistribution Analysis |
| Skeletal Muscle | 36 | 36 |
| (Quadriceps) | | |
| Skeletal Muscle | 33 | 36 |
| (Gastrocnemius) | | |
| Skeletal Muscle | 29 | 30 |
| (Diaphragm) | | |
| Skeletal Muscle | 35 | 36 |
| (Biceps) | | |
| Heart | 32 | 36 |
| Liver | 10 | 14 |
| Spleen | 10 | 12 |

### 8.2.1.4. Bulk Sequencing NGS Library Preparation

Cardiac muscle was dissected to isolate basal and apex subregions. For all biodistribution and transduction analyses, total DNA was extracted from tissue samples using MagMAX DNA Multi-Sample Ultra 2.0 Kit (Thermofisher). Samples were first homogenized in the supplied lysis buffer, then incubated at 65° C. with Proteinase K before the lysates were added to a plate for automated extraction using the KingFisher Apex instrument (Thermofisher). RNA was extracted using the MagMAX™ mirVana™ Total RNA Isolation Kit (Thermofisher) according to the manufacturer's recommendations, and was further treated with DNase I-XT (New England Biolabs) to remove any vector DNA contamination in the RNA sample. Reverse transcription was done with Protoscript II Reverse Transcriptase (New England Biolabs) utilizing primers that were specific to the vector transgene and included unique molecular identifiers (UMIs). Control reactions lacking the reverse transcriptase enzyme (−RT control) were also prepared. Finally, samples were prepared for next-generation sequencing by amplifying the transgene barcode regions with primers compatible with Illumina NGS platform and sequenced with NextSeq 2000 (Illumina).

### 8.2.1.5. Bulk Tissue NGS Sample Parsing and Analysis

After sequencing, the barcode tags were extracted from reads with the expected amplicon structure, and the abundance (number of reads, number of UMIs, or number of unique combinations of barcode and ID tags) of each barcode was recorded. Analyses were restricted to the set of barcodes that were present in the input plasmid sample, as measured by a separate sequencing assay that targeted the variant regions of the input plasmid sample.

To aggregate packaging replicates, the read counts from replicate virus production samples were summed. To aggregate biodistribution samples, read counts from samples from the same tissue or organ were summed. To aggregate transduction samples, the number of transduction events (measured by the number of unique barcode and ID tag combinations detected) from samples from the same tissue or organ were summed.

Virus packaging was calculated by normalizing aggregated production replicates to input plasmid abundance. Biodistribution and transduction of tissue were calculated by normalizing aggregated biodistribution or transduction samples to input virus abundance. The output was reported as fold change relative to the WT AAV9.

### 8.2.2. Results

Production efficiencies of variant capsids V1 and V5-V150 (having subcombinations of mutations of V1), along with a known RGD-insertion containing capsid variant, relative to WT AAV9 are summarized in Table 7A. The changes in biodistribution and transduction in various tissues in cynomolgus monkeys relative to WT AAV9 are summarized in Table 7B (biodistribution relative to WT AAV9) and Table 7C (transduction relative to WT AAV9). The changes in biodistribution and transduction in various mouse tissues relative to WT AAV9 are summarized in Table 7D (biodistribution relative to WT AAV9) and Table 7E (transduction relative to WT AAV9). The changes in in vitro transduction in various cells relative to WT AAV9 are summarized in Table 7F. All values are reported as the log 2 fold change relative to WT AAV9. A value of −6.00 indicates that the variant was not detected in the indicated tissue.

In sum, AAV capsid variant V1 showed an increase in biodistribution to skeletal and cardiac muscle tissues and a decrease in biodistribution to the liver relative to biodistribution associated with wildtype AAV9 in these tissues (Table 7B). AAV capsid variant V1 also displayed increased transduction in skeletal and cardiac muscle tissues, relative to transduction associated with wildtype AAV9 (Table 7C).

TABLE 7A
Production efficiency of variant virus particles relative to production
efficiency of WT AAV9 virus particles, in HEK293 cells in vitro.

| Variant # | Fold change relative to wtAAV9 |
|---|---|
| V1 | 0.27 |
| V5 | 0.81 |
| V6 | 0.63 |
| V7 | 0.57 |
| V8 | 1.06 |
| V9 | 1.08 |
| V10 | 0.20 |
| V11 | 0.37 |
| V12 | 0.20 |
| V13 | 0.65 |
| V14 | 0.70 |
| V15 | 0.44 |
| V16 | 0.30 |
| V17 | 0.82 |
| V18 | 0.76 |
| V19 | 0.31 |
| V20 | 0.98 |
| V21 | 0.27 |
| V22 | 0.56 |
| V23 | 1.68 |
| V24 | 0.04 |
| V25 | 0.38 |
| V26 | 0.48 |
| V27 | 0.24 |
| V28 | 0.74 |
| V29 | 1.35 |
| V30 | 0.09 |
| V31 | 0.83 |
| V32 | 0.49 |
| V33 | 0.92 |
| V34 | 1.30 |
| V35 | 0.73 |
| V36 | 0.06 |
| V37 | 0.92 |
| V38 | 0.27 |
| V39 | 0.61 |
| V40 | 0.56 |
| V41 | 1.44 |
| V42 | 0.66 |
| V43 | 0.40 |
| V44 | 1.18 |
| V45 | −0.44 |
| V46 | 0.28 |
| V47 | 0.77 |
| V48 | 0.99 |
| V49 | 1.12 |
| V50 | 1.22 |
| V51 | 0.95 |
| V52 | 0.78 |
| V53 | 0.44 |
| V54 | 1.33 |
| V55 | 0.01 |
| V56 | −0.14 |
| V57 | 0.93 |
| V58 | 0.30 |
| V59 | 0.30 |
| V60 | 0.92 |
| V61 | 0.64 |
| V62 | 0.87 |
| V63 | 0.99 |
| V64 | 0.68 |
| V65 | −0.26 |
| V66 | 0.13 |
| V67 | 0.59 |
| V68 | 0.91 |
| V69 | 0.52 |
| V70 | 0.67 |
| V71 | 1.66 |
| V72 | −0.18 |
| V73 | 1.38 |
| V74 | 0.36 |
| V75 | 0.84 |
| V76 | 1.03 |
| V77 | 1.09 |
| V78 | 1.26 |
| V79 | 0.36 |
| V80 | −0.51 |
| V81 | 0.14 |
| V82 | 0.67 |
| V83 | 0.98 |
| V84 | 1.51 |
| V85 | −0.12 |
| V86 | 0.45 |
| V87 | 0.31 |
| V88 | 0.81 |
| V89 | 0.86 |
| V90 | 0.64 |
| V91 | −0.08 |
| V92 | 0.22 |
| V93 | 0.75 |
| V94 | 0.64 |
| V95 | 1.06 |
| V96 | 0.46 |
| V97 | 0.69 |
| V98 | 0.30 |
| V99 | 1.46 |
| V100 | 0.77 |
| V101 | 0.01 |
| V102 | 0.31 |
| V103 | −0.12 |
| V104 | 1.06 |
| V105 | 0.67 |
| V106 | 0.65 |
| V107 | 0.81 |
| V108 | 0.84 |
| V109 | 0.75 |
| V110 | 0.14 |
| V111 | −0.17 |
| V112 | 0.89 |
| V113 | 1.12 |
| V114 | 0.50 |
| V115 | 0.50 |
| V116 | 1.22 |
| V117 | 0.80 |
| V118 | 0.93 |
| V119 | 0.04 |
| V120 | 0.73 |
| V121 | 0.86 |
| V122 | 0.29 |
| V123 | 0.09 |
| V124 | 0.85 |
| V125 | 0.58 |
| V126 | 0.94 |
| V127 | 0.28 |
| V128 | 0.19 |
| V129 | 0.89 |
| V130 | −0.12 |
| V131 | −0.18 |
| V132 | 0.89 |
| V133 | 0.17 |
| V134 | 1.40 |
| V135 | 0.53 |
| V136 | 1.70 |
| V137 | 0.08 |
| V138 | 0.49 |
| V139 | 0.63 |
| V140 | −0.37 |
| V141 | 0.66 |
| V142 | −0.54 |
| V143 | 0.18 |
| V144 | −0.37 |
| V145 | 0.05 |
| V146 | 0.78 |
| V147 | 0.88 |
| V148 | 0.48 |
| V149 | 0.71 |
| V150 | −0.13 |
| Known RGD-insertion containing | 0.11 |
| capsid variant | |

TABLE 7B
Fold change in biodistribution of virus particles comprising the indicated AAV capsid
variant polypeptide in cynomolgus monkey tissues, relative to the biodistribution
of virus particles comprising WT AAV9 (amino acid sequence - SEQ ID NO: 7; nucleotide
sequence - SEQ ID NO: 8) for the indicated tissues. “Aggregated” in relation
to a tissue is an average across all measured areas in the specified organ.

| | Tissue | | | | | | | |
|---|---|---|---|---|---|---|---|---|
| | | | Muscle | | | | | |
| Variant | Heart | Liver | (aggregated) | Spleen | Biceps | Diaphragm | Gastrocnemius | Quadriceps |
| V1 | 1.21 | −0.81 | 0.30 | −0.05 | −0.02 | 0.69 | 0.21 | 0.49 |
| V5 | 1.04 | −0.64 | 0.51 | −0.73 | 0.31 | 1.09 | −0.21 | 0.79 |
| V6 | 1.26 | −0.12 | 1.10 | −1.03 | 1.56 | 1.25 | −0.07 | 0.76 |
| V7 | −0.02 | −1.04 | 0.23 | 0.62 | 0.18 | 0.26 | −0.42 | 0.96 |
| V8 | 1.16 | −0.85 | 0.29 | −0.31 | −0.61 | 1.37 | −1.16 | 0.82 |
| V9 | 1.23 | −1.08 | 0.42 | −0.21 | 0.15 | 0.94 | 0.12 | 0.53 |
| V10 | −0.64 | −2.08 | −1.06 | −0.47 | −3.20 | −0.90 | −2.53 | −1.80 |
| V11 | 1.04 | −0.03 | 0.44 | 0.43 | −0.19 | 1.39 | −0.17 | 0.50 |
| V12 | 0.83 | −0.22 | 0.52 | −0.82 | −0.66 | 0.43 | 1.38 | 0.89 |
| V13 | 0.58 | 0.01 | 0.18 | −0.88 | 0.37 | 0.15 | 0.15 | 0.03 |
| V14 | 0.74 | −0.38 | 0.56 | −0.55 | −0.47 | 0.70 | 1.06 | 0.93 |
| V15 | 0.85 | 0.21 | 1.04 | −0.51 | 1.31 | 1.62 | −0.23 | 0.80 |
| V16 | −0.10 | −0.91 | 0.07 | −0.63 | 0.05 | 0.06 | 0.60 | 0.11 |
| V17 | 0.33 | −1.04 | −0.08 | 0.78 | −0.14 | −0.32 | 0.50 | −0.11 |
| V18 | 0.47 | 0.08 | 0.07 | −0.58 | 0.08 | −0.13 | 0.05 | 0.13 |
| V19 | 1.03 | −0.80 | −0.01 | −0.68 | 0.68 | 0.29 | 0.00 | −0.33 |
| V20 | 1.17 | −0.09 | 0.85 | −0.19 | 0.54 | 0.95 | 0.53 | 1.05 |
| V21 | 1.03 | −1.08 | 0.49 | −0.93 | 0.60 | 0.71 | 0.35 | 0.61 |
| V22 | 0.85 | −0.24 | 0.54 | 0.05 | 0.11 | 0.59 | −1.20 | 1.80 |
| V23 | 0.31 | −0.28 | 0.17 | 0.08 | −0.43 | 0.85 | −0.32 | 0.50 |
| V24 | 0.71 | −0.23 | 0.29 | −0.72 | −0.80 | 0.90 | 0.49 | 0.39 |
| V25 | 0.08 | −1.15 | −0.74 | 0.31 | −2.21 | 0.93 | −3.41 | −0.34 |
| V26 | 0.92 | −0.99 | 0.18 | −0.10 | −0.21 | 0.95 | −0.15 | 0.14 |
| V27 | 0.86 | −0.05 | 0.43 | 0.37 | −0.38 | 0.60 | 0.52 | 0.56 |
| V28 | 1.16 | 0.09 | 0.89 | −0.89 | 0.95 | 1.06 | −0.04 | 1.63 |
| V29 | 0.93 | −0.02 | 0.46 | −0.26 | 0.08 | 0.52 | 0.25 | 0.98 |
| V30 | 0.41 | 0.11 | −0.22 | −1.18 | −1.86 | −0.18 | 0.36 | −0.32 |
| V31 | 0.18 | −0.79 | −0.02 | 0.61 | −1.84 | 0.68 | 0.53 | −0.18 |
| V32 | 1.12 | 0.16 | 0.92 | −0.84 | 0.81 | 0.86 | 1.10 | 0.94 |
| V33 | 0.88 | −0.16 | 0.11 | −0.03 | 0.91 | 0.05 | −0.72 | 0.11 |
| V34 | 0.13 | −0.28 | −0.01 | −0.41 | −0.11 | −0.02 | 0.11 | 0.39 |
| V35 | −0.03 | −1.33 | −0.49 | 0.66 | −1.41 | 0.66 | −0.52 | −0.51 |
| V36 | 0.39 | −0.81 | 0.14 | 0.13 | 0.99 | −0.14 | −2.63 | 0.44 |
| V37 | 0.64 | −0.19 | 0.09 | −0.58 | −0.05 | 0.09 | −0.14 | 0.46 |
| V38 | 0.12 | −1.46 | −1.25 | 0.53 | −6.00 | −0.36 | −0.50 | −0.83 |
| V39 | 0.96 | −0.02 | 0.87 | −1.35 | 0.79 | 1.17 | 1.10 | −0.10 |
| V40 | 0.00 | −0.98 | −0.18 | 0.18 | 0.52 | 0.11 | −1.13 | −0.44 |
| V41 | 1.14 | 0.24 | 1.05 | −0.22 | 0.85 | 1.45 | 1.13 | 0.30 |
| V42 | 0.93 | 0.09 | 0.70 | −0.55 | 0.78 | 1.18 | 0.67 | 0.07 |
| V43 | 0.90 | −0.21 | 0.36 | −0.80 | −0.05 | −0.23 | 0.80 | 0.79 |
| V44 | 0.90 | −0.09 | 0.48 | −0.89 | 0.46 | 0.30 | −0.26 | 0.55 |
| V45 | 1.02 | −0.02 | 0.38 | −0.25 | −0.32 | 1.02 | 0.49 | 0.65 |
| V46 | −0.75 | −0.04 | −0.79 | −1.41 | −0.65 | 0.28 | −1.67 | −3.33 |
| V47 | 0.53 | −0.38 | 0.13 | −0.69 | −0.66 | 0.87 | −0.43 | 0.57 |
| V48 | 1.09 | −0.01 | 1.28 | −0.13 | 0.74 | 2.01 | 1.02 | 1.70 |
| V49 | 1.19 | 0.13 | 0.68 | 0.05 | −1.03 | 1.21 | 0.43 | 0.87 |
| V50 | −1.43 | −1.25 | −0.47 | −0.58 | −1.88 | −0.79 | −2.17 | −0.56 |
| V51 | 0.05 | −1.15 | −0.67 | 0.76 | −0.55 | 0.37 | −2.00 | −0.58 |
| V52 | 1.02 | 0.30 | 1.50 | −0.85 | −0.21 | 1.45 | 2.29 | 1.86 |
| V53 | 1.12 | −0.09 | 0.86 | −0.11 | 0.17 | 1.36 | 1.39 | 0.46 |
| V54 | 1.12 | 0.31 | 0.70 | −0.32 | 0.99 | 0.99 | −0.84 | 0.62 |
| V55 | −6.00 | −6.00 | −6.00 | −6.00 | −5.28 | −1.37 | 1.31 | −6.00 |
| V56 | 1.26 | −0.02 | 0.36 | −0.08 | −4.14 | 0.33 | 1.85 | −0.19 |
| V57 | 0.23 | −0.76 | −0.39 | 1.01 | −1.98 | 0.38 | −0.28 | 0.43 |
| V58 | 0.98 | 0.16 | 0.79 | −1.56 | 0.86 | 0.83 | 1.19 | 0.18 |
| V59 | 0.20 | −0.73 | −0.94 | 1.68 | −2.58 | −0.12 | −1.85 | −0.58 |
| V60 | 0.08 | −1.22 | −0.48 | 1.11 | −1.65 | −0.05 | 0.11 | −0.15 |
| V61 | 0.42 | −0.30 | 0.45 | −0.17 | 0.94 | 0.38 | −0.57 | 0.53 |
| V62 | 0.13 | −0.84 | −0.42 | 0.91 | −2.09 | −0.08 | 0.40 | −1.36 |
| V63 | 0.50 | −0.38 | −0.11 | −0.39 | 0.43 | −0.65 | −0.69 | 0.48 |
| V64 | 0.93 | −0.27 | 0.50 | −0.35 | 1.22 | 0.57 | −0.50 | 0.98 |
| V65 | −0.08 | −0.78 | −0.40 | −0.24 | −0.19 | −0.78 | −2.92 | 0.96 |
| V66 | −0.04 | −1.22 | −0.55 | −0.35 | −1.49 | −0.33 | 0.93 | −6.00 |
| V67 | 1.14 | −1.23 | 0.43 | 0.07 | 0.36 | 0.39 | 0.79 | 0.56 |
| V68 | 0.40 | −0.53 | 0.34 | 0.48 | −0.31 | 0.83 | 0.74 | −0.19 |
| V69 | 0.50 | −0.33 | 0.43 | −0.03 | 1.14 | 0.27 | −0.65 | 0.25 |
| V70 | 1.16 | 0.04 | 0.68 | −0.12 | 1.58 | 0.48 | −1.32 | 0.43 |
| V71 | −0.19 | −0.84 | −0.96 | −1.11 | −1.27 | −0.75 | −0.94 | −0.40 |
| V72 | −0.02 | −1.18 | 0.01 | 0.48 | −0.59 | 0.76 | −0.22 | 0.29 |
| V73 | 1.26 | 0.16 | 0.91 | −0.57 | 1.02 | 1.59 | 0.08 | 0.38 |
| V74 | 0.94 | 0.19 | 0.74 | −0.52 | 0.71 | 0.82 | −0.61 | 0.99 |
| V75 | −0.14 | −0.75 | −0.84 | −0.32 | −1.12 | −1.91 | −0.92 | −0.38 |
| V76 | 0.54 | −0.03 | 0.14 | −0.13 | 0.02 | 0.06 | 0.09 | 0.14 |
| V77 | 1.01 | −0.12 | 0.53 | 0.07 | 0.03 | 1.14 | 0.46 | 1.01 |
| V78 | 1.05 | 0.02 | 0.63 | 0.06 | −0.34 | 1.19 | 1.13 | 0.62 |
| V79 | 0.91 | 0.30 | 0.32 | −1.46 | 0.46 | 0.14 | −0.55 | 0.14 |
| V80 | 1.00 | −0.16 | 0.19 | 0.50 | 0.79 | 0.47 | −2.75 | 1.03 |
| V81 | 0.76 | −0.09 | 0.57 | −0.37 | −1.17 | 1.45 | 0.87 | 0.58 |
| V82 | −0.02 | −1.67 | −0.39 | 0.32 | −1.02 | −0.69 | −1.53 | 0.62 |
| V83 | 0.33 | −0.63 | 0.41 | −0.61 | 0.70 | 0.69 | −0.16 | 0.85 |
| V84 | −0.08 | −0.96 | −0.44 | −0.25 | −0.72 | −0.71 | 0.04 | −0.64 |
| V85 | 0.12 | −0.84 | −0.19 | 0.44 | 0.27 | −0.02 | −1.89 | 0.19 |
| V86 | −0.39 | 0.11 | −0.41 | −0.62 | −1.20 | −1.37 | 0.03 | 0.08 |
| V87 | 0.49 | −0.51 | 0.43 | 0.02 | 0.53 | 0.39 | 0.11 | 1.10 |
| V88 | 0.91 | −0.09 | 0.72 | −0.15 | 0.99 | 1.61 | −0.07 | 0.50 |
| V89 | 0.51 | −0.15 | 0.13 | −0.01 | −0.78 | −0.02 | 0.24 | 0.57 |
| V90 | 1.01 | 0.26 | 0.40 | −1.39 | −1.28 | 1.04 | 0.25 | 0.48 |
| V91 | −0.03 | −1.10 | −0.30 | 0.66 | −1.59 | 0.61 | −0.18 | −0.60 |
| V92 | −0.10 | −0.82 | 0.23 | −0.46 | 0.35 | −0.38 | 1.12 | −1.03 |
| V93 | 0.78 | 0.05 | 0.42 | 0.16 | 1.70 | 0.91 | −2.30 | −0.78 |
| V94 | 0.40 | −0.31 | 0.28 | −0.14 | 0.61 | 0.57 | −0.76 | 0.07 |
| V95 | 0.24 | −0.18 | −0.07 | 0.40 | −5.14 | 1.00 | −0.54 | 0.47 |
| V96 | 0.18 | −0.24 | 0.56 | 0.39 | 0.77 | 1.08 | −1.28 | 0.93 |
| V97 | −0.58 | −1.05 | −0.31 | 0.28 | −1.83 | 0.54 | −1.02 | 0.51 |
| V98 | 0.36 | −0.26 | 0.42 | −0.67 | 0.07 | 0.36 | 1.33 | −1.42 |
| V99 | 0.66 | −0.40 | 0.51 | −0.95 | 0.96 | 0.32 | 0.22 | 0.49 |
| V100 | 1.05 | 0.12 | 0.72 | −0.08 | 0.33 | 1.53 | 0.78 | 0.23 |
| V101 | −0.53 | −0.71 | −0.90 | 1.37 | −3.10 | −1.26 | −1.16 | 0.26 |
| V102 | 0.64 | −0.69 | −0.38 | 0.61 | −1.64 | −0.70 | −0.59 | 1.04 |
| V103 | 0.19 | −1.02 | −0.45 | 2.06 | −0.44 | −1.25 | 0.09 | −1.04 |
| V104 | −0.08 | −1.02 | −0.41 | −0.18 | 0.29 | −1.04 | −1.64 | −1.27 |
| V105 | 0.06 | −1.07 | −0.40 | 0.03 | 0.22 | −0.03 | −2.35 | −0.83 |
| V106 | 1.04 | 0.23 | 0.71 | −0.08 | 0.99 | 1.18 | 0.21 | 0.33 |
| V107 | 0.45 | −0.57 | 0.10 | −0.09 | −0.25 | 0.94 | 0.07 | −0.11 |
| V108 | 1.23 | 0.15 | 0.63 | −0.78 | 0.85 | 0.97 | 0.65 | 0.56 |
| V109 | 1.09 | 0.19 | 0.80 | −0.15 | −1.97 | 0.89 | 1.42 | 1.19 |
| V110 | 1.13 | −0.20 | 0.30 | −1.01 | −2.00 | 0.74 | 0.22 | 0.63 |
| V111 | −0.06 | −1.14 | −0.23 | 1.22 | 0.27 | 0.08 | 0.14 | −1.10 |
| V112 | 0.89 | 0.20 | 0.83 | −0.91 | 1.22 | 1.53 | 0.16 | 0.45 |
| V113 | 1.12 | 0.18 | 0.59 | −0.82 | 0.96 | 1.10 | 0.19 | 0.47 |
| V114 | 0.37 | −0.37 | 0.31 | −0.15 | 0.03 | 0.17 | −0.28 | 0.25 |
| V115 | 0.23 | −0.24 | 0.38 | 1.04 | 0.85 | 0.76 | −0.28 | 0.21 |
| V116 | 0.70 | −0.07 | 0.36 | 0.82 | 0.30 | 0.61 | −0.28 | 0.42 |
| V117 | 0.28 | −0.96 | −0.50 | 0.45 | −0.56 | −1.12 | 0.56 | −2.86 |
| V118 | −0.68 | −1.63 | −0.55 | 0.67 | 0.52 | −0.50 | −6.00 | −0.75 |
| V119 | −0.40 | −0.77 | −0.88 | 0.51 | −0.30 | −0.60 | −2.29 | −0.87 |
| V120 | 0.36 | −0.53 | −0.56 | −0.24 | −0.63 | −0.24 | −0.73 | −1.19 |
| V121 | 0.67 | −0.15 | −0.05 | −0.11 | −0.19 | 0.53 | −2.54 | 0.46 |
| V122 | −0.23 | −1.20 | −0.58 | 0.63 | −0.81 | −0.72 | −0.32 | −1.31 |
| V123 | −0.01 | −1.32 | −0.11 | −0.20 | −1.12 | 1.05 | −0.05 | −0.71 |
| V124 | 1.09 | 0.15 | 0.67 | −0.34 | −0.03 | 1.34 | 0.71 | 0.75 |
| V125 | 0.35 | −0.26 | 0.12 | −0.10 | −0.08 | 0.33 | 0.17 | 0.07 |
| V126 | −0.01 | −1.17 | −0.51 | −0.60 | 0.11 | −1.19 | −2.85 | 0.41 |
| V127 | 0.41 | −0.31 | 0.39 | −0.22 | 1.33 | 0.29 | −1.28 | −0.09 |
| V128 | 0.31 | −0.09 | 0.24 | −0.28 | 0.52 | 0.74 | 0.61 | −1.64 |
| V129 | 1.13 | 0.33 | 0.72 | −0.38 | 0.85 | 1.11 | 0.98 | −0.22 |
| V130 | −0.18 | −0.65 | 0.60 | 0.52 | 1.10 | 0.68 | 0.15 | 0.14 |
| V131 | −0.06 | −0.34 | −0.55 | −0.20 | −0.53 | −1.23 | −1.81 | −0.51 |
| V132 | −0.07 | −1.01 | −0.37 | 0.06 | −1.30 | 0.01 | −0.75 | −0.50 |
| V133 | −0.06 | −0.17 | −0.29 | 0.36 | 0.26 | −0.53 | −1.57 | 0.49 |
| V134 | 0.41 | −0.51 | 0.16 | −0.10 | 0.19 | 0.37 | 0.28 | 0.52 |
| V135 | 0.11 | −0.02 | 0.23 | −0.57 | 0.63 | −0.13 | −0.46 | 0.53 |
| V136 | 1.10 | 0.38 | 0.29 | −0.69 | 0.28 | 0.90 | −0.08 | −0.68 |
| V137 | 0.60 | −0.29 | −0.11 | 0.29 | −0.29 | 0.55 | −0.92 | −0.47 |
| V138 | 0.27 | −0.51 | −0.14 | −0.43 | −0.77 | 0.17 | 0.66 | −0.37 |
| V139 | 0.66 | −0.28 | 0.25 | −0.55 | −1.98 | 0.88 | 0.20 | 0.84 |
| V140 | 0.01 | −0.07 | −0.33 | 0.39 | −0.30 | 0.25 | −1.01 | −0.25 |
| V141 | 0.20 | −0.40 | 0.46 | 0.92 | 0.24 | 0.29 | 0.64 | 0.57 |
| V142 | −0.63 | −0.58 | 0.09 | 0.16 | 0.09 | 0.25 | 0.38 | 0.15 |
| V143 | 0.11 | 0.06 | 0.06 | −0.59 | −0.23 | 0.16 | 0.33 | 0.13 |
| V144 | 0.00 | 0.15 | 0.52 | 0.38 | 0.80 | −0.12 | −0.48 | 1.63 |
| V145 | 0.33 | 0.00 | 0.29 | −0.77 | 0.50 | −0.08 | 1.12 | 0.06 |
| V146 | 0.10 | 0.19 | −0.22 | −0.52 | −3.43 | 0.14 | 1.38 | −6.00 |
| V147 | 0.53 | −0.10 | 0.02 | 0.18 | −0.43 | 0.51 | −0.53 | 0.00 |
| V148 | −0.07 | −0.05 | −0.11 | −0.14 | −0.18 | −1.05 | 0.74 | −2.25 |
| V149 | 0.01 | 0.06 | 0.28 | 0.16 | −0.55 | 0.63 | 0.94 | −0.72 |
| V150 | −1.36 | −1.06 | −0.31 | −0.83 | −0.44 | −0.57 | −2.63 | 0.44 |
| Known | −0.64 | −2.21 | −0.88 | 0.42 | −0.75 | −0.39 | −1.35 | −1.65 |
| RGD- | | | | | | | | |
| insertion | | | | | | | | |
| containing | | | | | | | | |
| capsid | | | | | | | | |
| variant | | | | | | | | |

TABLE 7C
Fold change in transduction of virus particles comprising the indicated AAV capsid
variant polypeptide in cynomolgus monkey tissues, relative to the transduction
of virus particles comprising WT AAV9 (amino acid sequence - SEQ ID NO: 7; nucleotide
sequence - SEQ ID NO: 8) for the indicated tissues. “Aggregated” in relation
to a tissue is an average across all measured areas in the specified organ.

| | Tissue | | | | | | | |
|---|---|---|---|---|---|---|---|---|
| | | | Muscle | | | | | |
| Variant | Heart | Liver | (aggregated) | Spleen | Biceps | Diaphragm | Gastrocnemius | Quadriceps |
| V1 | 1.14 | −1.01 | 1.95 | −0.25 | 1.72 | 2.10 | 1.79 | 1.67 |
| V5 | 1.15 | −1.77 | 1.71 | −6.00 | 1.07 | 1.93 | 1.35 | 1.19 |
| V6 | 0.66 | −0.28 | 1.60 | −6.00 | 1.48 | 1.78 | 1.73 | 0.88 |
| V7 | 0.34 | −0.61 | 1.03 | −6.00 | 1.16 | 1.09 | 0.35 | 1.14 |
| V8 | 1.33 | −1.68 | 1.84 | −6.00 | 2.36 | 1.71 | 0.98 | 2.26 |
| V9 | 1.06 | −0.95 | 1.90 | 2.42 | 1.44 | 2.06 | 1.63 | 1.76 |
| V10 | −1.43 | −2.23 | 0.18 | −6.00 | −0.92 | 0.50 | 0.59 | −1.43 |
| V11 | 1.15 | 0.47 | 2.06 | −6.00 | 0.31 | 2.23 | 1.65 | 2.29 |
| V12 | 0.44 | −0.60 | 2.02 | −6.00 | 1.79 | 2.12 | 1.94 | 1.92 |
| V13 | 0.45 | −0.12 | 0.46 | −6.00 | 0.57 | 0.49 | 0.11 | 0.39 |
| V14 | 0.75 | 0.14 | 2.12 | −6.00 | 1.49 | 2.12 | 2.63 | 1.96 |
| V15 | 1.15 | −0.16 | 1.97 | 4.33 | 1.84 | 1.97 | 2.03 | 2.04 |
| V16 | −1.23 | −1.29 | 0.87 | −6.00 | 0.30 | 1.00 | 0.75 | 0.88 |
| V17 | 0.08 | −1.07 | 1.25 | 2.50 | 1.19 | 1.13 | 1.74 | 1.35 |
| V18 | 0.02 | 0.15 | 0.34 | −6.00 | 0.37 | 0.34 | 0.24 | 0.35 |
| V19 | 1.38 | −2.16 | 2.23 | −6.00 | 1.31 | 2.40 | 1.87 | 2.26 |
| V20 | 0.79 | −0.70 | 2.05 | −6.00 | 1.41 | 2.28 | 1.51 | 1.79 |
| V21 | 1.17 | −0.96 | 1.97 | −6.00 | 1.24 | 2.19 | 1.68 | 1.80 |
| V22 | 0.14 | −0.70 | 1.35 | −6.00 | 0.34 | 1.42 | 2.02 | 0.87 |
| V23 | 0.34 | 0.60 | 1.45 | 2.73 | 0.83 | 1.29 | 1.34 | 1.92 |
| V24 | 1.27 | −0.17 | 2.12 | −6.00 | 1.85 | 2.22 | 1.91 | 2.18 |
| V25 | 0.27 | −0.87 | 1.29 | −6.00 | 0.28 | 1.50 | 0.74 | 1.37 |
| V26 | 0.88 | −1.43 | 1.80 | −6.00 | 1.41 | 2.04 | 1.24 | 1.46 |
| V27 | 0.98 | −0.30 | 1.75 | −6.00 | 1.84 | 1.99 | 1.06 | 1.13 |
| V28 | −0.91 | −1.66 | 0.67 | −6.00 | −0.56 | 0.94 | 0.78 | 0.09 |
| V29 | 1.42 | 0.53 | 2.44 | −6.00 | 1.83 | 2.65 | 2.52 | 1.93 |
| V30 | 1.06 | −0.54 | 0.47 | −6.00 | −0.39 | 0.27 | 0.70 | 1.00 |
| V31 | 0.41 | −0.30 | 0.94 | −6.00 | −0.60 | 0.90 | 1.77 | 1.11 |
| V32 | 1.19 | 0.28 | 2.42 | −6.00 | 2.02 | 2.50 | 3.11 | 1.70 |
| V33 | 0.88 | −0.07 | 1.77 | −6.00 | 1.19 | 1.84 | 1.57 | 2.00 |
| V34 | 0.65 | −0.23 | 0.43 | −6.00 | 0.41 | 0.52 | −0.50 | 0.58 |
| V35 | 0.73 | −0.45 | 1.55 | −6.00 | 1.45 | 1.32 | 1.84 | 2.07 |
| V36 | 0.06 | 0.00 | −0.02 | −6.00 | 0.67 | −0.17 | −0.99 | 0.12 |
| V37 | 0.54 | −0.13 | 0.70 | 1.03 | 0.28 | 0.75 | 1.04 | 0.52 |
| V38 | 0.50 | −2.30 | 1.05 | −6.00 | 0.75 | 1.19 | 0.19 | 0.98 |
| V39 | 1.01 | −0.13 | 2.14 | −6.00 | 1.75 | 2.08 | 2.76 | 1.93 |
| V40 | 0.88 | −0.33 | 1.24 | −6.00 | 0.46 | 1.12 | 1.79 | 1.51 |
| V41 | 0.77 | 0.25 | 2.21 | −6.00 | 2.21 | 2.34 | 2.44 | 1.41 |
| V42 | 0.54 | −0.19 | 1.90 | −6.00 | 1.66 | 2.24 | 1.25 | 0.97 |
| V43 | 0.17 | −0.53 | 1.31 | −6.00 | 2.05 | 1.18 | 1.45 | 1.07 |
| V44 | 1.43 | −0.08 | 2.66 | −6.00 | 1.61 | 3.00 | 2.48 | 1.41 |
| V45 | 0.81 | 0.26 | 2.15 | −6.00 | 2.51 | 2.32 | 2.21 | 1.39 |
| V46 | −2.92 | 0.14 | −1.42 | −6.00 | −2.57 | −1.68 | −1.07 | −0.76 |
| V47 | 1.16 | 0.49 | 1.56 | −6.00 | 1.04 | 1.64 | 1.34 | 1.58 |
| V48 | 0.79 | −0.26 | 2.13 | −6.00 | 1.89 | 2.06 | 2.36 | 2.43 |
| V49 | 1.11 | 0.06 | 2.68 | −6.00 | 2.00 | 2.83 | 2.15 | 2.77 |
| V50 | −1.29 | −1.37 | −1.05 | −6.00 | −2.16 | −1.47 | 0.36 | −0.27 |
| V51 | 0.98 | −0.69 | 1.24 | −6.00 | 1.29 | 0.91 | 1.45 | 1.86 |
| V52 | −0.30 | 0.27 | 1.97 | −6.00 | 1.69 | 2.12 | 2.13 | 1.44 |
| V53 | 1.15 | −0.13 | 2.16 | −6.00 | 1.19 | 2.34 | 1.20 | 2.42 |
| V54 | 1.45 | 0.48 | 2.35 | −6.00 | 1.44 | 2.51 | 2.01 | 2.35 |
| V55 | −6.00 | −6.00 | −6.00 | −6.00 | −0.64 | 0.32 | −0.14 | −0.09 |
| V56 | 1.10 | 0.15 | 2.39 | −6.00 | 1.65 | 2.57 | 2.44 | 2.10 |
| V57 | −0.01 | −0.17 | 1.25 | −6.00 | 0.47 | 0.65 | 3.03 | 1.02 |
| V58 | 0.99 | 0.22 | 2.51 | −6.00 | 2.72 | 2.60 | 2.11 | 2.29 |
| V59 | 0.88 | −0.46 | 0.37 | −6.00 | −0.77 | 0.58 | 0.15 | 0.27 |
| V60 | −0.21 | −1.85 | 1.20 | −6.00 | 0.32 | 1.05 | 0.76 | 2.07 |
| V61 | 0.12 | 0.01 | 1.33 | −6.00 | 0.27 | 1.29 | 1.44 | 1.75 |
| V62 | 0.41 | −0.06 | 1.45 | −6.00 | 0.30 | 1.46 | 1.03 | 1.99 |
| V63 | 0.71 | −0.63 | 0.48 | −6.00 | 0.69 | 0.29 | 0.77 | 0.66 |
| V64 | 0.46 | −0.10 | 2.09 | 4.12 | 2.06 | 2.13 | 2.09 | 1.95 |
| V65 | 0.30 | −0.44 | 0.23 | −6.00 | −0.05 | 0.22 | 0.19 | 0.35 |
| V66 | 0.38 | −1.34 | 0.97 | −6.00 | 1.23 | 0.91 | 0.56 | 0.87 |
| V67 | 1.05 | −1.11 | 2.06 | −6.00 | 2.51 | 2.08 | 1.87 | 1.83 |
| V68 | 0.49 | −0.26 | 1.67 | −6.00 | 1.27 | 1.83 | 1.51 | 1.44 |
| V69 | 0.19 | −0.08 | 1.30 | −6.00 | 0.94 | 1.30 | 1.11 | 1.54 |
| V70 | 0.54 | −0.07 | 1.78 | −6.00 | 0.09 | 2.00 | 1.89 | 1.59 |
| V71 | 0.20 | −1.10 | 0.48 | −6.00 | 0.43 | 0.47 | 1.14 | −0.10 |
| V72 | −0.86 | −1.27 | 0.64 | −6.00 | 1.53 | −0.14 | 0.72 | 1.51 |
| V73 | 1.24 | 0.82 | 2.07 | −6.00 | 1.61 | 2.42 | 1.41 | 1.24 |
| V74 | 0.64 | 0.09 | 1.99 | −6.00 | 1.56 | 1.91 | 2.13 | 2.31 |
| V75 | −0.25 | −1.26 | 0.82 | −6.00 | 2.81 | 0.05 | 0.44 | 0.23 |
| V76 | 0.57 | 0.17 | 0.72 | −0.17 | 0.32 | 0.78 | 0.81 | 0.63 |
| V77 | 1.54 | 0.44 | 2.62 | −6.00 | 2.83 | 2.65 | 2.30 | 2.60 |
| V78 | 0.85 | 0.38 | 2.35 | −6.00 | 1.43 | 2.68 | 1.89 | 1.77 |
| V79 | 1.66 | 0.19 | 2.86 | −6.00 | 0.06 | 3.01 | 3.15 | 2.90 |
| V80 | 1.46 | −0.17 | 2.72 | −6.00 | 1.77 | 2.79 | 3.11 | 2.38 |
| V81 | 0.77 | −0.12 | 1.29 | −6.00 | 1.18 | 1.47 | 0.99 | 0.85 |
| V82 | 0.95 | −1.24 | 1.22 | −6.00 | 1.89 | 1.05 | 1.19 | 1.41 |
| V83 | −1.44 | −1.81 | −0.47 | −6.00 | −1.23 | −0.12 | −1.63 | −0.74 |
| V84 | 0.41 | −1.35 | 0.07 | −6.00 | −0.38 | 0.22 | 0.05 | −0.32 |
| V85 | 0.68 | −0.43 | 0.65 | −6.00 | 0.15 | 0.41 | 0.88 | 1.19 |
| V86 | −1.04 | −0.40 | −0.90 | −6.00 | −1.15 | −1.05 | −2.23 | −0.11 |
| V87 | 0.21 | −0.46 | 1.29 | 2.83 | 1.28 | 1.24 | 1.82 | 1.26 |
| V88 | 0.81 | 0.31 | 2.16 | −6.00 | 1.75 | 2.29 | 2.16 | 1.99 |
| V89 | 0.36 | −0.97 | 1.13 | −6.00 | 0.50 | 1.10 | 2.10 | 0.61 |
| V90 | 0.74 | 0.28 | 2.14 | −6.00 | 1.20 | 2.37 | 2.07 | 1.49 |
| V91 | 0.42 | −0.83 | 0.60 | −6.00 | −0.17 | 0.32 | 1.53 | 0.64 |
| V92 | −0.42 | 0.36 | 0.15 | −6.00 | −0.29 | 0.16 | −0.14 | 0.35 |
| V93 | 2.07 | 0.89 | 2.40 | −6.00 | 3.06 | 2.30 | 2.69 | 1.88 |
| V94 | −0.22 | −0.20 | 1.33 | −6.00 | 0.07 | 1.54 | 1.39 | 1.05 |
| V95 | 0.26 | 0.14 | 0.78 | −6.00 | −0.09 | 1.13 | 0.16 | 0.31 |
| V96 | 0.41 | −0.09 | 0.56 | −6.00 | −0.31 | 0.68 | 0.14 | 0.83 |
| V97 | 0.42 | −1.69 | 1.43 | −6.00 | 1.04 | 1.20 | 2.18 | 1.53 |
| V98 | 0.50 | 0.30 | 1.02 | −6.00 | −0.30 | 1.11 | 0.91 | 1.45 |
| V99 | 0.86 | −0.82 | 1.49 | −6.00 | 1.25 | 1.43 | 1.21 | 1.90 |
| V100 | 0.94 | 0.11 | 2.44 | −6.00 | 2.44 | 2.51 | 1.98 | 2.50 |
| V101 | 0.37 | −0.37 | 1.13 | −6.00 | 0.61 | 1.06 | 1.65 | 1.34 |
| V102 | −1.20 | −0.14 | 1.13 | −6.00 | 0.42 | 1.24 | −0.25 | 1.58 |
| V103 | 0.38 | −0.17 | 0.69 | −6.00 | −1.14 | 0.40 | 1.28 | 1.50 |
| V104 | −0.62 | −1.46 | 0.82 | −6.00 | 0.06 | 1.19 | −0.76 | −0.04 |
| V105 | 0.10 | 0.22 | 0.65 | −6.00 | 0.59 | 0.74 | 0.60 | 0.41 |
| V106 | 1.10 | −0.40 | 2.26 | −6.00 | 1.55 | 2.44 | 1.80 | 2.19 |
| V107 | 0.27 | −0.57 | 1.54 | −6.00 | 1.30 | 1.54 | 1.92 | 1.53 |
| V108 | 1.45 | 0.53 | 2.51 | −6.00 | 1.24 | 2.73 | 1.45 | 2.58 |
| V109 | 1.74 | 0.29 | 2.33 | −6.00 | 1.60 | 2.51 | 2.71 | 1.79 |
| V110 | 0.62 | −1.92 | 1.29 | −6.00 | 0.47 | 1.54 | 1.13 | 0.57 |
| V111 | 0.82 | −0.47 | 1.31 | −6.00 | 1.28 | 1.22 | 0.21 | 1.75 |
| V112 | 0.72 | −0.26 | 1.95 | −6.00 | 1.15 | 2.15 | 2.09 | 1.58 |
| V113 | 0.78 | 0.28 | 2.55 | −6.00 | 1.91 | 2.71 | 2.75 | 2.24 |
| V114 | 0.36 | −0.18 | 0.95 | −6.00 | 0.92 | 0.98 | 0.94 | 0.94 |
| V115 | −0.03 | −0.45 | 1.15 | −6.00 | 1.26 | 1.12 | 1.66 | 0.97 |
| V116 | 0.15 | −0.18 | 1.60 | −6.00 | 2.55 | 1.22 | 2.07 | 1.73 |
| V117 | 0.38 | −0.89 | 0.60 | −6.00 | 1.13 | 0.45 | 0.36 | 0.77 |
| V118 | 0.01 | −0.74 | 0.54 | −6.00 | 0.89 | 0.01 | 0.81 | 1.37 |
| V119 | 0.34 | −0.89 | 1.27 | −6.00 | 1.42 | 0.81 | 1.48 | 2.07 |
| V120 | 0.71 | −0.59 | 1.51 | −6.00 | 0.72 | 1.51 | 1.12 | 2.03 |
| V121 | 1.74 | 1.32 | 2.64 | −6.00 | 2.55 | 2.59 | 2.52 | 2.87 |
| V122 | 0.03 | −1.01 | 0.99 | −6.00 | 1.08 | 0.94 | 1.34 | 1.03 |
| V123 | 0.26 | −0.93 | 0.88 | −6.00 | 0.68 | 0.89 | 0.98 | 0.91 |
| V124 | 1.88 | 0.50 | 2.70 | −6.00 | 1.91 | 2.87 | 2.87 | 2.38 |
| V125 | 0.23 | −0.33 | 0.52 | −6.00 | 0.15 | 0.52 | 0.16 | 0.70 |
| V126 | 0.14 | −0.56 | 0.75 | −6.00 | −0.18 | 0.98 | −0.54 | 0.95 |
| V127 | 0.40 | 0.40 | 1.44 | −6.00 | 1.83 | 1.49 | 1.42 | 0.82 |
| V128 | 0.21 | 0.34 | 0.87 | −6.00 | 0.65 | 0.72 | 0.90 | 1.40 |
| V129 | 1.20 | −0.24 | 2.16 | −6.00 | 1.85 | 2.23 | 2.18 | 2.10 |
| V130 | 0.45 | 0.61 | 1.26 | −6.00 | 1.32 | 1.31 | −0.08 | 1.65 |
| V131 | −1.79 | −0.64 | −0.09 | −6.00 | −0.11 | 0.14 | −0.51 | −0.62 |
| V132 | 0.29 | −0.81 | 1.50 | 1.57 | 1.77 | 1.45 | 1.23 | 1.65 |
| V133 | 0.28 | −0.18 | 1.02 | −6.00 | 1.88 | 0.66 | 0.55 | 1.56 |
| V134 | −0.07 | −0.79 | 1.13 | −6.00 | 1.92 | 0.93 | 1.29 | 0.83 |
| V135 | −0.32 | 0.22 | −0.22 | 2.18 | −1.17 | −0.19 | 0.33 | −0.14 |
| V136 | 0.78 | −0.21 | 2.27 | −6.00 | 2.98 | 2.37 | 2.12 | 1.29 |
| V137 | 0.33 | 0.13 | 1.18 | −6.00 | 1.17 | 1.36 | 1.28 | 0.50 |
| V138 | −1.44 | −0.87 | 0.58 | −6.00 | 0.49 | 0.51 | −0.75 | 1.01 |
| V139 | 0.90 | −0.61 | 1.52 | −6.00 | 1.28 | 1.68 | 1.14 | 1.37 |
| V140 | −0.51 | −0.50 | −1.21 | −6.00 | −6.00 | −1.39 | −1.05 | −0.42 |
| V141 | −0.03 | 0.33 | 1.33 | −6.00 | 1.38 | 1.34 | 1.19 | 1.37 |
| V142 | 0.28 | −0.61 | −0.14 | −6.00 | 0.22 | −0.70 | 1.02 | −0.13 |
| V143 | 0.74 | −0.05 | −0.20 | −6.00 | 0.45 | −0.12 | −0.62 | −1.00 |
| V144 | 0.90 | −0.58 | 0.85 | −6.00 | −0.33 | 1.02 | 1.18 | 0.12 |
| V145 | 0.51 | −0.69 | −0.67 | −6.00 | −0.07 | −1.01 | −1.47 | −0.35 |
| V146 | −1.24 | 0.22 | 0.12 | −6.00 | 0.43 | 0.33 | 0.35 | −1.66 |
| V147 | 0.76 | 0.36 | 1.02 | −6.00 | 0.56 | 0.84 | 0.93 | 1.68 |
| V148 | 0.12 | −0.11 | 0.41 | −6.00 | 1.28 | 0.47 | −2.60 | 0.40 |
| V149 | 0.37 | −0.48 | 0.06 | 3.83 | −0.06 | 0.14 | −1.02 | 0.16 |
| V150 | −0.66 | −0.56 | −0.49 | −6.00 | −2.11 | −0.95 | −0.60 | 0.82 |
| Known | −1.41 | −2.73 | 0.23 | −6.00 | 0.49 | 0.20 | −0.13 | −0.38 |
| RGD- | | | | | | | | |
| insertion | | | | | | | | |
| containing | | | | | | | | |
| capsid | | | | | | | | |
| variant | | | | | | | | |

TABLE 7D
Fold change in biodistribution of virus particles comprising the indicated AAV capsid variant polypeptide
in mouse tissues, relative to the biodistribution of virus particles comprising WT AAV9 (amino acid
sequence - SEQ ID NO: 7; nucleotide sequence - SEQ ID NO: 8) for the indicated tissues. “Aggregated”
in relation to a tissue is an average across all measured areas in the specified organ.

| | Tissue | | | | | | | |
|---|---|---|---|---|---|---|---|---|
| | | | Muscle | | | | | |
| Variant | Heart | Liver | (aggregated) | Spleen | Diaphragm | Gastrocnemius | Quadriceps | Triceps |
| V1 | 0.05 | −1.38 | −0.99 | 0.49 | −1.08 | −0.88 | −1.06 | −1.00 |
| V5 | 0.29 | −1.62 | −1.99 | −1.47 | −0.84 | −4.70 | −1.10 | −6.00 |
| V6 | −0.31 | 0.14 | −0.11 | −1.71 | −0.33 | −1.80 | 1.21 | −0.54 |
| V7 | −0.20 | −1.93 | −0.08 | −2.68 | −1.99 | −1.83 | 1.17 | 0.43 |
| V8 | −0.15 | −2.15 | −2.11 | −0.06 | −1.13 | −6.00 | −0.82 | −6.00 |
| V9 | −0.56 | −1.65 | −1.13 | 0.91 | −1.83 | −1.88 | −0.19 | −0.97 |
| V10 | −1.23 | −3.98 | −5.52 | −2.19 | −3.66 | −6.00 | −6.00 | −6.00 |
| V11 | 0.67 | 0.57 | 0.39 | −0.42 | 0.74 | 0.47 | 1.09 | −1.48 |
| V12 | 0.85 | 0.15 | −0.35 | −1.95 | 0.40 | 0.62 | −6.00 | −3.15 |
| V13 | −0.11 | 0.01 | −0.29 | −2.05 | −0.69 | −0.10 | 0.36 | −0.74 |
| V14 | −0.03 | −0.16 | −0.83 | −0.79 | −0.59 | 0.60 | −6.00 | −6.00 |
| V15 | 0.15 | 0.86 | −0.07 | −1.12 | 0.51 | −6.00 | 0.36 | 0.15 |
| V16 | −0.38 | −1.78 | −1.96 | −3.00 | −0.61 | −6.00 | −1.30 | −6.00 |
| V17 | −0.55 | −2.24 | −1.33 | −1.20 | −2.47 | −6.00 | 0.76 | −3.36 |
| V18 | −0.13 | 0.09 | −0.27 | −1.77 | −0.43 | −0.44 | −0.28 | −0.01 |
| V19 | −0.03 | −1.60 | −2.11 | 1.05 | −0.25 | −6.00 | −6.00 | −6.00 |
| V20 | 0.56 | 0.12 | −0.18 | −0.40 | −0.94 | 0.75 | 0.17 | −1.45 |
| V21 | −0.26 | −2.67 | −0.98 | −2.05 | −1.89 | −0.79 | −6.00 | 0.01 |
| V22 | 0.65 | 0.49 | −0.29 | −2.09 | 0.07 | 0.12 | −1.16 | −0.75 |
| V23 | 0.44 | −0.62 | −0.28 | −0.44 | 0.26 | 0.82 | −2.02 | −6.00 |
| V24 | 0.92 | −0.50 | 0.67 | −3.45 | 0.54 | 1.53 | −6.00 | 0.70 |
| V25 | −0.67 | −1.96 | −1.61 | −1.92 | −2.93 | −6.00 | −6.00 | 0.04 |
| V26 | 0.28 | −1.50 | −0.94 | 0.00 | −0.14 | −1.00 | −0.46 | −5.55 |
| V27 | 0.71 | 0.44 | −0.54 | −0.26 | −0.33 | −2.27 | −2.18 | 0.40 |
| V28 | −0.35 | −0.05 | −0.36 | −2.27 | −0.20 | −0.52 | 0.94 | −6.00 |
| V29 | 0.13 | 0.32 | −0.18 | −1.39 | 0.68 | −1.49 | 0.89 | −6.00 |
| V30 | −1.29 | −1.20 | −1.21 | −2.82 | −0.07 | −6.00 | −1.31 | −1.59 |
| V31 | −0.63 | −1.33 | −0.35 | −1.47 | −0.22 | −0.43 | −6.00 | 0.35 |
| V32 | −0.07 | 0.86 | 0.65 | −2.17 | 0.08 | 0.57 | 0.06 | 1.32 |
| V33 | 0.35 | 0.09 | −0.20 | −0.45 | −0.36 | −2.97 | 0.77 | 0.08 |
| V34 | −0.80 | −1.48 | −2.16 | −0.28 | −2.01 | −2.83 | −3.14 | −1.52 |
| V35 | −0.42 | −2.31 | −1.71 | −1.20 | −1.30 | −6.00 | −1.34 | −1.25 |
| V36 | −0.56 | −1.17 | 1.33 | 0.89 | 0.09 | 1.45 | −6.00 | 2.44 |
| V37 | −0.40 | −0.43 | −0.50 | −0.75 | −0.40 | −0.01 | −0.28 | −1.64 |
| V38 | −0.21 | −2.93 | −1.78 | −2.19 | −0.82 | −6.00 | −6.00 | −1.08 |
| V39 | −0.34 | −0.46 | −0.95 | −1.75 | −1.91 | −6.00 | 1.24 | −6.00 |
| V40 | −0.13 | −1.82 | −1.79 | −2.90 | −1.64 | −0.93 | −1.46 | −6.00 |
| V41 | 1.08 | 1.12 | 0.28 | −2.38 | 0.44 | 1.51 | −0.65 | −6.00 |
| V42 | 0.43 | 0.84 | 0.71 | −3.11 | 0.75 | 1.66 | −6.00 | 0.41 |
| V43 | 1.01 | −1.01 | −0.24 | −2.48 | −0.38 | 0.66 | −6.00 | −0.24 |
| V44 | 0.45 | −0.20 | −1.22 | −2.29 | −0.95 | −0.13 | −2.57 | −3.49 |
| V45 | 0.46 | 0.14 | −2.57 | −1.57 | −1.84 | −6.00 | −6.00 | −1.63 |
| V46 | 0.79 | 0.27 | −0.59 | −4.16 | 0.08 | 0.09 | −0.87 | −6.00 |
| V47 | −0.12 | −0.94 | −0.20 | −4.27 | −0.61 | −1.06 | −0.49 | 0.62 |
| V48 | 1.00 | −0.31 | 0.51 | −1.85 | 0.69 | 1.45 | 0.21 | −1.97 |
| V49 | 0.81 | 0.79 | −0.07 | −0.82 | 0.16 | −0.18 | 0.27 | −0.52 |
| V50 | −0.04 | −1.13 | −1.30 | −5.01 | −0.28 | −2.55 | −1.58 | −1.96 |
| V51 | 0.59 | −2.53 | −0.84 | −2.14 | −2.26 | −4.91 | 1.20 | −2.24 |
| V52 | 1.33 | 0.25 | −1.21 | −4.12 | 0.23 | −1.21 | −6.00 | −6.00 |
| V53 | 1.10 | 0.28 | −1.51 | 0.24 | −0.30 | −2.23 | −1.34 | −6.00 |
| V54 | 0.11 | 1.19 | −0.09 | 0.27 | −0.02 | −1.92 | 0.21 | 0.42 |
| V55 | 0.30 | −1.36 | 0.27 | −2.24 | −5.07 | 1.37 | −6.00 | 0.95 |
| V56 | 0.34 | −0.38 | 0.62 | −0.72 | −0.18 | −0.75 | 2.34 | −0.59 |
| V57 | −0.34 | −0.56 | −1.15 | −0.73 | −0.41 | −6.00 | −6.00 | −0.23 |
| V58 | 1.10 | 0.69 | 0.37 | −6.00 | 1.27 | 1.29 | −6.00 | −4.36 |
| V59 | 0.30 | −0.18 | −0.14 | 0.47 | −0.43 | 1.48 | −6.00 | −6.00 |
| V60 | −0.35 | −2.03 | 0.14 | −1.80 | −0.99 | −0.76 | 0.58 | 0.90 |
| V61 | −0.25 | −0.43 | −1.76 | −4.03 | −0.65 | −6.00 | −6.00 | −1.28 |
| V62 | −1.09 | −0.92 | 0.25 | −1.29 | −0.47 | 1.10 | −0.34 | 0.07 |
| V63 | −1.37 | −0.71 | −0.03 | 0.46 | −1.05 | 0.23 | 0.52 | −0.01 |
| V64 | 0.82 | 0.18 | −0.85 | −1.15 | −0.77 | 0.38 | −4.01 | −2.35 |
| V65 | −1.27 | −1.19 | −1.00 | −0.84 | 0.06 | −0.27 | −6.00 | −6.00 |
| V66 | 0.74 | −2.17 | −1.64 | −1.95 | −0.65 | −6.00 | −6.00 | −0.98 |
| V67 | 0.88 | −1.96 | −0.83 | −0.41 | −1.61 | −0.23 | −0.64 | −1.04 |
| V68 | −0.08 | −0.07 | −0.75 | −2.11 | −0.99 | 0.06 | −0.04 | −4.51 |
| V69 | 0.07 | 0.09 | 1.05 | −2.60 | −0.04 | 0.44 | 1.90 | 1.41 |
| V70 | 0.78 | −0.12 | −0.42 | −3.50 | −0.34 | −6.00 | 1.48 | −5.64 |
| V71 | −1.06 | −2.66 | −0.81 | −1.23 | −1.30 | −6.00 | 1.02 | −1.90 |
| V72 | 0.57 | −1.41 | 0.39 | −2.60 | −1.32 | 0.37 | 0.75 | 0.97 |
| V73 | 0.34 | 0.69 | 0.26 | −1.04 | 0.53 | −0.40 | −6.00 | 1.10 |
| V74 | 0.44 | 0.86 | 0.31 | −2.97 | 1.01 | −0.37 | −0.22 | 0.20 |
| V75 | −0.70 | −1.14 | −2.53 | −1.70 | −1.50 | −6.00 | −6.00 | −1.91 |
| V76 | −0.13 | 0.13 | −0.41 | −0.50 | −0.30 | −0.45 | −0.37 | −0.50 |
| V77 | 1.14 | 0.07 | −0.52 | −1.28 | 0.34 | −0.47 | −0.75 | −2.43 |
| V78 | −0.09 | 0.98 | −0.32 | 0.00 | 0.62 | −0.60 | −6.00 | −0.40 |
| V79 | −1.80 | 0.09 | −0.37 | −2.21 | −0.35 | 1.15 | −6.00 | −6.00 |
| V80 | 0.46 | 0.10 | −1.46 | −1.41 | 0.40 | −6.00 | −6.00 | −6.00 |
| V81 | −0.04 | −0.52 | −0.89 | −2.90 | −0.74 | −1.03 | 0.41 | −6.00 |
| V82 | −0.16 | −2.64 | −1.77 | −2.32 | −1.20 | −0.65 | −3.90 | −6.00 |
| V83 | 0.15 | −0.87 | −0.67 | −2.14 | −0.23 | 0.37 | −1.46 | −6.00 |
| V84 | −1.04 | −2.38 | −2.15 | 0.50 | −2.63 | −6.00 | −0.37 | −3.01 |
| V85 | −0.35 | −0.87 | 0.76 | −0.15 | −1.65 | 0.17 | −6.00 | 2.22 |
| V86 | 0.00 | 0.57 | −0.95 | 0.59 | −0.53 | −1.48 | 0.34 | −6.00 |
| V87 | 0.17 | −0.45 | −0.62 | −3.21 | −0.45 | −0.35 | 0.40 | −6.00 |
| V88 | 0.64 | 0.07 | −0.18 | −1.30 | −0.07 | 0.22 | −0.34 | −0.69 |
| V89 | 0.18 | 0.38 | −0.49 | −0.49 | −0.82 | −0.39 | −6.00 | 0.35 |
| V90 | −0.08 | 0.69 | 0.17 | −0.73 | 0.09 | −0.75 | 1.86 | −6.00 |
| V91 | −0.99 | −1.28 | 1.02 | 0.64 | −0.61 | 0.71 | 0.90 | 1.92 |
| V92 | 0.12 | −2.10 | −0.73 | −1.82 | −1.80 | 0.66 | −6.00 | −1.18 |
| V93 | −0.17 | 1.07 | −0.79 | −1.94 | −0.36 | −0.35 | −0.17 | −6.00 |
| V94 | −0.14 | 0.28 | −0.73 | −5.40 | −0.05 | 0.07 | −1.35 | −6.00 |
| V95 | −0.14 | 0.49 | −1.39 | −1.68 | −0.31 | −0.66 | −6.00 | −6.00 |
| V96 | 0.47 | 0.15 | 0.26 | 1.40 | −0.13 | −2.68 | 1.89 | −0.42 |
| V97 | 0.23 | −1.53 | 0.27 | −0.93 | 0.06 | −6.00 | 2.07 | −1.13 |
| V98 | 0.75 | −0.23 | −0.36 | −1.41 | −0.31 | −0.16 | 0.16 | −1.21 |
| V99 | −0.17 | −0.75 | −0.90 | −2.38 | −0.45 | −0.58 | −6.00 | −0.78 |
| V100 | 0.24 | 1.12 | 0.03 | −3.33 | 0.43 | 0.78 | −0.10 | −2.78 |
| V101 | −0.17 | −1.25 | 0.47 | −1.11 | −0.58 | −6.00 | 2.00 | 0.66 |
| V102 | 0.77 | 0.07 | −3.20 | −0.47 | −1.34 | −6.00 | −6.00 | −6.00 |
| V103 | 0.06 | −0.38 | −0.05 | 1.87 | −0.91 | 1.03 | −0.42 | −0.83 |
| V104 | −0.65 | −2.14 | 0.06 | −3.27 | −0.77 | −0.10 | 1.78 | −6.00 |
| V105 | −0.23 | −1.36 | −0.64 | −1.87 | 0.13 | −6.00 | 0.40 | −1.69 |
| V106 | 0.27 | 1.03 | 0.16 | −2.33 | 0.23 | −0.04 | −0.27 | 0.47 |
| V107 | 0.12 | −0.89 | −0.51 | −1.38 | −0.86 | −0.79 | −0.15 | −0.26 |
| V108 | 0.63 | 0.75 | 0.61 | −3.35 | 0.61 | −6.00 | 2.32 | −0.79 |
| V109 | 0.88 | 1.06 | 1.07 | −0.53 | 0.76 | 2.29 | −0.42 | −0.13 |
| V110 | −0.58 | −0.20 | −1.11 | −2.39 | −0.24 | −6.00 | −6.00 | −0.30 |
| V111 | 0.96 | −1.12 | −0.43 | 0.99 | −0.98 | −6.00 | 1.67 | −6.00 |
| V112 | 0.19 | 0.42 | −0.13 | −6.00 | 0.39 | −1.44 | 0.98 | −1.72 |
| V113 | 0.62 | 0.99 | 0.17 | −0.49 | 0.32 | 0.81 | 0.62 | −2.45 |
| V114 | 0.61 | 0.33 | 0.31 | −1.93 | −0.39 | −0.61 | −6.00 | 1.61 |
| V115 | 0.44 | 0.33 | −0.05 | 0.39 | −0.45 | 0.93 | 0.15 | −1.86 |
| V116 | −0.18 | 0.40 | −0.87 | −2.20 | −0.92 | 0.68 | −6.00 | −6.00 |
| V117 | −0.56 | −1.48 | −1.79 | −3.16 | −0.41 | −6.00 | −1.54 | −4.09 |
| V118 | −0.50 | −3.09 | −1.42 | 1.24 | −0.87 | −6.00 | −2.36 | −0.61 |
| V119 | −0.11 | −0.55 | −2.65 | 0.33 | −0.79 | −6.00 | −6.00 | −6.00 |
| V120 | −0.05 | −0.78 | −0.98 | −1.55 | −0.40 | 0.18 | −4.97 | −5.06 |
| V121 | 0.71 | −0.80 | −2.01 | −5.99 | −0.15 | −6.00 | −6.00 | −6.00 |
| V122 | 0.34 | −1.49 | −1.40 | −1.69 | −1.52 | −6.00 | 0.33 | −2.64 |
| V123 | 0.07 | −1.70 | −0.35 | −1.32 | −0.03 | −6.00 | 1.45 | −6.00 |
| V124 | 0.84 | 0.75 | −0.63 | −3.93 | −0.52 | −0.24 | −6.00 | −0.23 |
| V125 | 0.39 | −0.20 | −0.65 | −3.05 | −0.26 | −2.69 | 0.51 | −1.51 |
| V126 | −0.13 | −2.67 | −0.62 | −3.23 | −1.45 | −0.37 | −0.47 | −0.39 |
| V127 | 0.53 | −0.55 | −0.14 | −2.00 | −0.23 | −1.52 | 1.32 | −1.35 |
| V128 | 0.19 | −0.43 | −0.80 | 0.44 | 0.47 | −0.44 | −6.00 | −5.81 |
| V129 | 0.45 | 0.68 | 1.08 | −1.52 | 0.64 | 2.07 | −0.26 | 0.74 |
| V130 | 0.67 | 0.23 | 0.72 | 0.44 | −0.57 | 1.00 | −0.23 | 1.48 |
| V131 | −0.03 | −0.50 | −0.19 | 0.89 | −1.00 | −6.00 | 1.60 | −0.73 |
| V132 | −1.24 | −2.41 | −1.82 | −2.82 | −3.06 | −0.46 | −6.00 | −2.08 |
| V133 | 0.09 | −0.71 | −0.13 | −2.06 | −0.10 | 1.38 | −6.00 | −6.00 |
| V134 | 0.81 | −0.27 | −1.39 | −2.36 | −0.40 | −6.00 | −0.49 | −2.92 |
| V135 | −0.37 | −0.08 | −0.65 | −1.34 | −0.32 | −0.35 | −1.40 | −0.94 |
| V136 | 0.52 | 0.75 | −0.28 | −4.20 | 0.35 | −0.41 | 0.59 | −6.00 |
| V137 | 0.33 | 0.12 | 0.18 | −3.65 | 0.33 | 1.15 | −6.00 | −0.33 |
| V138 | 0.54 | −0.61 | −1.45 | −1.31 | −0.11 | −6.00 | −6.00 | −1.36 |
| V139 | 0.48 | −0.47 | 0.18 | −3.11 | 0.06 | 1.15 | −6.00 | 0.00 |
| V140 | −0.61 | 0.28 | −1.66 | −0.35 | 0.20 | −6.00 | −6.00 | −6.00 |
| V141 | 0.44 | 0.43 | 0.22 | 0.69 | −0.12 | 1.44 | −6.00 | −0.29 |
| V142 | 0.35 | −1.17 | −0.33 | −0.37 | −2.03 | 0.76 | −6.00 | 0.08 |
| V143 | 0.13 | −0.11 | 0.08 | −0.89 | 0.32 | 0.36 | 1.06 | −6.00 |
| V144 | −0.22 | 0.44 | 0.30 | 0.56 | 0.72 | 1.63 | −6.00 | −6.00 |
| V145 | 0.14 | −0.71 | 0.52 | −4.98 | 0.81 | −6.00 | 2.16 | −1.44 |
| V146 | −4.63 | −0.86 | 1.27 | −1.39 | −0.85 | 1.39 | 1.81 | 1.74 |
| V147 | 0.51 | 0.14 | 0.72 | 1.07 | 0.41 | −1.27 | 1.80 | 0.88 |
| V148 | −0.29 | 0.22 | 0.19 | −1.47 | 0.47 | 0.32 | −6.00 | 0.64 |
| V149 | 0.25 | 0.13 | −1.54 | 0.72 | −0.17 | −5.23 | −6.00 | −1.65 |
| V150 | −0.11 | −1.83 | 0.05 | −2.53 | −4.21 | 0.34 | −0.91 | 1.07 |
| Known | 1.85 | −1.49 | 1.48 | −2.12 | 0.28 | 1.55 | 2.09 | 1.69 |
| RGD- | | | | | | | | |
| insertion | | | | | | | | |
| containing | | | | | | | | |
| capsid | | | | | | | | |
| variant | | | | | | | | |

TABLE 7E
Fold change in transduction of virus particles comprising the indicated AAV capsid variant polypeptide
in mouse tissues, relative to the transduction of virus particles comprising WT AAV9 (amino acid
sequence - SEQ ID NO: 7; nucleotide sequence - SEQ ID NO: 8) for the indicated tissues. “Aggregated”
in relation to a tissue is an average across all measured areas in the specified organ.

| | Tissue | | | | | | | |
|---|---|---|---|---|---|---|---|---|
| | | | Muscle | | | | | |
| Variant | Heart | Liver | (aggregated) | Spleen | Diaphragm | Gastrocnemius | Quadriceps | Triceps |
| V1 | 0.26 | −2.15 | −0.05 | −2.55 | −0.09 | 0.13 | 0.01 | −0.35 |
| V5 | 0.13 | −1.79 | 0.99 | −6.00 | 0.15 | 2.64 | −6.00 | −6.00 |
| V6 | −0.53 | −0.55 | −0.37 | −1.80 | −1.21 | 0.28 | −6.00 | 1.01 |
| V7 | −0.48 | −2.06 | −0.36 | −3.79 | −0.09 | −1.88 | −0.06 | −0.15 |
| V8 | 0.11 | −3.16 | −0.50 | −3.92 | −0.34 | −1.43 | 0.65 | −6.00 |
| V9 | −0.13 | −2.30 | 0.34 | −3.40 | 0.28 | 0.74 | −0.22 | 0.33 |
| V10 | −1.95 | −6.00 | −0.19 | −2.20 | −2.94 | −6.00 | −1.53 | 2.40 |
| V11 | −0.23 | −0.23 | −0.55 | −2.23 | −0.17 | −0.48 | −0.57 | −6.00 |
| V12 | 0.96 | −0.10 | 0.99 | −1.58 | 1.51 | 0.68 | 0.60 | −1.17 |
| V13 | −0.47 | −0.35 | −0.29 | −1.68 | −0.56 | 0.16 | −0.45 | −0.22 |
| V14 | −0.05 | −1.17 | 0.08 | −6.00 | −0.04 | 1.06 | −0.44 | −6.00 |
| V15 | 0.17 | 0.10 | −0.71 | 0.78 | 0.09 | −1.81 | −6.00 | −1.09 |
| V16 | −0.19 | −2.58 | −0.03 | −3.15 | −0.25 | 1.14 | −1.16 | −6.00 |
| V17 | 0.46 | −2.51 | 0.10 | −2.55 | 0.25 | −0.64 | 0.18 | 0.40 |
| V18 | −0.22 | −0.09 | −0.43 | −1.19 | −0.73 | −0.10 | −0.12 | −0.61 |
| V19 | 0.40 | −1.26 | 1.40 | −2.97 | 1.19 | 1.32 | 0.02 | 2.57 |
| V20 | 0.83 | −0.88 | 0.64 | −1.46 | 0.71 | 1.03 | −0.21 | 0.44 |
| V21 | 0.60 | −2.18 | 0.38 | −3.00 | 0.26 | −0.03 | 0.99 | 0.48 |
| V22 | 0.06 | −1.65 | 0.11 | −2.06 | 0.53 | 0.17 | −0.07 | −6.00 |
| V23 | 0.62 | −0.66 | 0.10 | −1.00 | −0.52 | 0.75 | 0.41 | −0.10 |
| V24 | 1.21 | −0.65 | 0.79 | −1.55 | 1.25 | 0.36 | 0.59 | −0.50 |
| V25 | 0.11 | −2.18 | −0.46 | −1.88 | −0.07 | −0.39 | −0.48 | −6.00 |
| V26 | 0.42 | −2.12 | −0.12 | −1.65 | −0.17 | −0.16 | 0.12 | −0.24 |
| V27 | 0.50 | −1.01 | −0.05 | −1.83 | 0.49 | −0.18 | −1.01 | −1.78 |
| V28 | −1.27 | −1.11 | −1.41 | −2.84 | −0.84 | −6.00 | −1.43 | −1.20 |
| V29 | 0.85 | 0.46 | 0.82 | 0.14 | 0.86 | 0.68 | 0.76 | 0.99 |
| V30 | −0.02 | −0.22 | −0.67 | −0.09 | 0.23 | −1.18 | −6.00 | −6.00 |
| V31 | 0.47 | −0.94 | 0.50 | −0.60 | 0.36 | 1.27 | −0.11 | −0.47 |
| V32 | 1.22 | 0.62 | 0.13 | −1.03 | 0.14 | −0.13 | 0.96 | −1.40 |
| V33 | 0.69 | −0.19 | 1.14 | −0.92 | −0.51 | 2.40 | 1.90 | −6.00 |
| V34 | −0.69 | −1.72 | −1.18 | −4.51 | −1.12 | −3.02 | −0.94 | −0.29 |
| V35 | −0.29 | −2.60 | −0.01 | −3.05 | −0.10 | 0.72 | −1.87 | −0.05 |
| V36 | −0.30 | −1.82 | −1.18 | −3.19 | −0.03 | −6.00 | −6.00 | −6.00 |
| V37 | −0.24 | −0.50 | −0.29 | −1.57 | −0.46 | 0.48 | −1.02 | −0.92 |
| V38 | −0.18 | −2.86 | −0.91 | −6.00 | −1.75 | −6.00 | −6.00 | 1.47 |
| V39 | 0.59 | −0.50 | −1.02 | −0.86 | −0.86 | −0.95 | −6.00 | −0.22 |
| V40 | 0.12 | −0.96 | 0.10 | 0.14 | −0.52 | 1.04 | 0.08 | −0.69 |
| V41 | 0.21 | 0.44 | 0.71 | −1.71 | 1.19 | 1.10 | −1.72 | −1.49 |
| V42 | −0.28 | 0.24 | 0.04 | −3.71 | −1.13 | 0.10 | 1.45 | −0.49 |
| V43 | −0.02 | −1.53 | −0.52 | −6.00 | −0.59 | 0.32 | −0.77 | −6.00 |
| V44 | 0.70 | −0.06 | 0.50 | −0.54 | 0.86 | 0.67 | −0.64 | −0.41 |
| V45 | 0.08 | −0.68 | 0.93 | −1.00 | 1.17 | −1.09 | 1.41 | 1.22 |
| V46 | −0.96 | −0.57 | −0.11 | −3.85 | 0.54 | −6.00 | 0.72 | −6.00 |
| V47 | 0.26 | −0.75 | 0.18 | −0.44 | −0.38 | −0.27 | 1.55 | −0.55 |
| V48 | 0.46 | −0.54 | −0.24 | −0.16 | −0.36 | 0.55 | −0.76 | −1.53 |
| V49 | 1.17 | −0.32 | 0.14 | −2.98 | 0.18 | 0.51 | 0.59 | −6.00 |
| V50 | −0.13 | −1.91 | −0.90 | −4.02 | 0.14 | −2.53 | −6.00 | −6.00 |
| V51 | 0.70 | −1.52 | 0.60 | −6.00 | 1.51 | −0.59 | −1.09 | −6.00 |
| V52 | 0.98 | 0.16 | 0.49 | −0.58 | 0.77 | 0.68 | 0.60 | −6.00 |
| V53 | 1.10 | 0.20 | 1.11 | −0.37 | 0.58 | 2.12 | −6.00 | 1.63 |
| V54 | 0.07 | 0.64 | 0.99 | −1.47 | 1.11 | 1.61 | 0.52 | −1.83 |
| V55 | −0.55 | −1.12 | 0.08 | 0.78 | −6.00 | 1.89 | 0.06 | −6.00 |
| V56 | 1.40 | 0.10 | 1.53 | −0.05 | 0.95 | 0.27 | 3.23 | 0.00 |
| V57 | −0.24 | −1.00 | −0.51 | −1.59 | −0.82 | 0.09 | 0.18 | −6.00 |
| V58 | 0.21 | 0.31 | −0.38 | −2.26 | 0.13 | 0.23 | −6.00 | −6.00 |
| V59 | −0.31 | −0.87 | 0.11 | −1.63 | 0.27 | −1.14 | 1.36 | −6.00 |
| V60 | 0.05 | −3.00 | −0.53 | −1.76 | −0.31 | −0.05 | −0.77 | −6.00 |
| V61 | 0.44 | −0.41 | 0.03 | −0.13 | 0.33 | 0.10 | −0.14 | −1.50 |
| V62 | 1.02 | −0.63 | 0.26 | 0.61 | 1.19 | −6.00 | −6.00 | 0.24 |
| V63 | −0.02 | −1.30 | −0.21 | −2.22 | −1.29 | 0.13 | 0.77 | −0.14 |
| V64 | 0.92 | −0.12 | 0.24 | −0.42 | 0.53 | −1.02 | 1.07 | −1.29 |
| V65 | 0.04 | 0.20 | −0.91 | −3.33 | −0.16 | −0.84 | −6.00 | −6.00 |
| V66 | 0.02 | −2.38 | −0.89 | −3.64 | −0.47 | −1.15 | −6.00 | −0.42 |
| V67 | 0.91 | −2.63 | 0.29 | −2.07 | 0.63 | −1.17 | −0.67 | 1.15 |
| V68 | 0.34 | −0.47 | 0.57 | −0.80 | 0.67 | 0.16 | 1.07 | 0.04 |
| V69 | 0.09 | −0.03 | 0.19 | 0.08 | −0.51 | 0.20 | −1.11 | 1.71 |
| V70 | 0.60 | 0.15 | 1.04 | −2.19 | 1.13 | 2.04 | −0.79 | −6.00 |
| V71 | −1.16 | −2.22 | −1.08 | −2.73 | −0.73 | −0.24 | −6.00 | −6.00 |
| V72 | −0.50 | −1.45 | −0.45 | −1.29 | −0.97 | 0.78 | −1.89 | −1.66 |
| V73 | 0.32 | 0.16 | 0.28 | −1.11 | 0.53 | 1.00 | −1.00 | −6.00 |
| V74 | 0.50 | −0.03 | 0.35 | −0.78 | 0.80 | 0.25 | −6.00 | 0.56 |
| V75 | −0.13 | 0.07 | −0.49 | −0.63 | 0.30 | −1.60 | −1.10 | −6.00 |
| V76 | 0.05 | −0.13 | 0.01 | −1.13 | −0.05 | 0.44 | −0.11 | −0.59 |
| V77 | 1.18 | 0.28 | 1.22 | −0.23 | 1.18 | 1.59 | 1.17 | 0.55 |
| V78 | 0.16 | −0.06 | 0.71 | −1.85 | 0.74 | 1.13 | 0.82 | −0.95 |
| V79 | 0.74 | 0.70 | 0.37 | −0.64 | 0.94 | 0.27 | −0.23 | −6.00 |
| V80 | 0.85 | −0.25 | 0.59 | −0.25 | 0.75 | 1.39 | −0.43 | −6.00 |
| V81 | −0.09 | −0.70 | 0.42 | −2.32 | 0.48 | 0.17 | 1.08 | −0.69 |
| V82 | −0.14 | −2.18 | −0.04 | −3.12 | 0.04 | −2.22 | 0.60 | 0.51 |
| V83 | −1.68 | −1.86 | −0.82 | −2.83 | −1.25 | 0.47 | −6.00 | −1.61 |
| V84 | −0.62 | −2.71 | −0.80 | −5.47 | −0.57 | −0.66 | −2.48 | −0.67 |
| V85 | −0.14 | −1.56 | 0.03 | 0.24 | 0.19 | −0.22 | 0.28 | −0.49 |
| V86 | −0.05 | −0.76 | −1.68 | −0.84 | −6.00 | −0.35 | −0.44 | −6.00 |
| V87 | 0.81 | −0.38 | 0.37 | −1.48 | −0.64 | 0.01 | 1.77 | 0.41 |
| V88 | 0.58 | −0.34 | 0.24 | −1.40 | 0.59 | 0.50 | −0.32 | −2.09 |
| V89 | 0.45 | −0.46 | 0.04 | −0.29 | −0.14 | −0.13 | −6.00 | 1.49 |
| V90 | 1.08 | 0.21 | 0.65 | −2.78 | −0.61 | −0.29 | −0.79 | 2.76 |
| V91 | 0.44 | −1.73 | −0.67 | −0.67 | −0.68 | 0.23 | −6.00 | −1.04 |
| V92 | 0.22 | −0.99 | 0.18 | −4.15 | 0.43 | −0.66 | 0.42 | 0.06 |
| V93 | 0.44 | 0.64 | 1.30 | −2.22 | 2.17 | 0.86 | −6.00 | −6.00 |
| V94 | −0.30 | 0.31 | −0.16 | −0.35 | 0.23 | −1.18 | −0.10 | −0.45 |
| V95 | 1.20 | −0.94 | −1.94 | −1.36 | −0.78 | −6.00 | −6.00 | −6.00 |
| V96 | 0.02 | −0.21 | −0.09 | −0.76 | −0.86 | −1.27 | −1.77 | 1.92 |
| V97 | 0.59 | −1.27 | 0.67 | 0.55 | −0.24 | 1.67 | 1.16 | −0.93 |
| V98 | 0.27 | −0.11 | 0.36 | −0.13 | 0.52 | 0.11 | 0.61 | −0.17 |
| V99 | 0.12 | −1.20 | 0.00 | −1.28 | −0.11 | 0.80 | −1.29 | −0.48 |
| V100 | 0.71 | 0.95 | 0.22 | −0.02 | 0.08 | 0.78 | −0.42 | 0.13 |
| V101 | 1.01 | −2.12 | 1.34 | −0.67 | −0.09 | 0.24 | 1.32 | 3.28 |
| V102 | 0.21 | −1.02 | −0.70 | −2.45 | −1.87 | −0.96 | 0.54 | −0.23 |
| V103 | 0.69 | −0.60 | 0.50 | −0.84 | 0.89 | 0.80 | −1.02 | −0.79 |
| V104 | −0.27 | −1.87 | −0.12 | −0.74 | 0.62 | −1.05 | −6.00 | −0.33 |
| V105 | 0.24 | −1.99 | 0.55 | −1.10 | 1.07 | −1.19 | 1.30 | −6.00 |
| V106 | 1.10 | 0.38 | 1.37 | −1.26 | 0.65 | 1.88 | 1.38 | 1.95 |
| V107 | 0.58 | −0.77 | 0.44 | −2.04 | 0.72 | 0.17 | 0.23 | 0.11 |
| V108 | 1.00 | 0.74 | 0.91 | −3.21 | 0.83 | 1.74 | −0.22 | 0.01 |
| V109 | 0.45 | 0.29 | 0.35 | −6.00 | 0.97 | −0.58 | 0.50 | −6.00 |
| V110 | 0.34 | −1.44 | −0.97 | −3.40 | −0.23 | −0.91 | −6.00 | −6.00 |
| V111 | 0.72 | −1.16 | −0.06 | −1.80 | 0.78 | −1.31 | −6.00 | −0.59 |
| V112 | 0.19 | −0.02 | −0.87 | −0.35 | −1.09 | −1.50 | −0.42 | −0.19 |
| V113 | 0.85 | 0.65 | 1.51 | −0.50 | 0.25 | 0.31 | 1.07 | 3.52 |
| V114 | 0.30 | −0.16 | −0.14 | 0.38 | −1.10 | −0.78 | −1.28 | 1.76 |
| V115 | 0.27 | −0.46 | −0.02 | −0.54 | 0.41 | 0.14 | −6.00 | −0.13 |
| V116 | 0.98 | 0.38 | 1.59 | 0.80 | −0.01 | −1.10 | 3.72 | 0.63 |
| V117 | −0.02 | −0.98 | −0.54 | −1.74 | −0.38 | −1.06 | −1.56 | 0.26 |
| V118 | −0.82 | −4.42 | −1.67 | −6.00 | −0.51 | −6.00 | −6.00 | −6.00 |
| V119 | 0.10 | −1.44 | −0.69 | −2.53 | −0.53 | −0.04 | −1.12 | −6.00 |
| V120 | 0.50 | −0.61 | 0.55 | −1.38 | 0.36 | 0.31 | 0.91 | 0.94 |
| V121 | 0.69 | 0.13 | 0.99 | −1.68 | 0.90 | 1.62 | 0.31 | 0.54 |
| V122 | 0.40 | −1.93 | 0.52 | −0.26 | −0.51 | 1.84 | 0.09 | −0.68 |
| V123 | 1.32 | −1.63 | 0.75 | −1.92 | −0.01 | 0.57 | 2.39 | −6.00 |
| V124 | 0.89 | 0.69 | 0.77 | −0.88 | 1.08 | 0.75 | −0.63 | 0.82 |
| V125 | −0.17 | −0.76 | −0.70 | −0.88 | −0.74 | −0.05 | −2.14 | −0.91 |
| V126 | 0.05 | −1.85 | 0.60 | −1.75 | 0.67 | −0.43 | 1.66 | −0.70 |
| V127 | 1.18 | −0.31 | 0.55 | 0.17 | 0.31 | 0.76 | 1.26 | −0.52 |
| V128 | −0.89 | −0.64 | 0.25 | −1.04 | −2.05 | −0.14 | 1.36 | 1.59 |
| V129 | 0.71 | 0.27 | −0.63 | −0.64 | −1.47 | −0.15 | 0.35 | −1.42 |
| V130 | −0.32 | 0.00 | −0.06 | −0.70 | 0.62 | −1.80 | −0.30 | −1.07 |
| V131 | −0.34 | −0.17 | −2.29 | −1.13 | −6.00 | −0.22 | −6.00 | −6.00 |
| V132 | −0.85 | −2.16 | −1.11 | −1.11 | −0.48 | −1.98 | −1.07 | −6.00 |
| V133 | −1.28 | −0.38 | −0.06 | −0.84 | −1.07 | −1.16 | 1.92 | −6.00 |
| V134 | 0.03 | −0.56 | 0.12 | −0.25 | 0.28 | −0.19 | 0.57 | −0.78 |
| V135 | −0.84 | −0.25 | −0.71 | −1.06 | −0.17 | −0.96 | −2.46 | −1.24 |
| V136 | 0.42 | 0.73 | −0.24 | −1.47 | −1.89 | 1.02 | −6.00 | 0.75 |
| V137 | −0.03 | −0.02 | 0.50 | −1.92 | −0.75 | 1.38 | 1.07 | 0.30 |
| V138 | 0.01 | −0.65 | 0.17 | −0.96 | 0.63 | −1.47 | 0.62 | −0.74 |
| V139 | 0.61 | 0.20 | 0.21 | 0.14 | 0.36 | 0.10 | 0.19 | −0.17 |
| V140 | −0.09 | −0.18 | −0.50 | −1.66 | 0.66 | −6.00 | −6.00 | −6.00 |
| V141 | 0.40 | −0.23 | 0.62 | 0.47 | 0.62 | 1.29 | −0.67 | 0.14 |
| V142 | −0.07 | −0.88 | 0.69 | −1.64 | 0.52 | 1.85 | −0.65 | −6.00 |
| V143 | −0.17 | 0.09 | −1.82 | 0.63 | −2.24 | −0.34 | −6.00 | −6.00 |
| V144 | −0.45 | 0.19 | 1.14 | −0.39 | 2.20 | −6.00 | −6.00 | 0.02 |
| V145 | 0.22 | −0.03 | 0.46 | −0.67 | 0.24 | 0.41 | 1.32 | −0.45 |
| V146 | −1.28 | −0.41 | 0.57 | −0.36 | −0.59 | 0.90 | 1.40 | 1.05 |
| V147 | 0.50 | −0.32 | −0.52 | −0.94 | −0.14 | −0.45 | −1.54 | −1.31 |
| V148 | −1.43 | −0.37 | −0.21 | −1.00 | −0.64 | −1.32 | 0.18 | 1.00 |
| V149 | 0.09 | 0.43 | −0.13 | 0.55 | −0.05 | −1.31 | 0.77 | −0.59 |
| V150 | −0.50 | −1.17 | 0.03 | −0.58 | −0.81 | 1.42 | −0.40 | −6.00 |
| Known | 1.90 | −2.49 | 2.71 | −3.62 | 2.23 | 3.19 | 2.78 | 2.89 |
| RGD- | | | | | | | | |
| insertion | | | | | | | | |
| containing | | | | | | | | |
| capsid | | | | | | | | |
| variant | | | | | | | | |

TABLE 7F
Fold change in transduction of virus particles comprising the
indicated AAV capsid variant polypeptide in various cells
in vitro, relative to the transduction of virus particles
comprising WT AAV9 (amino acid sequence - SEQ ID NO: 7; nucleotide
sequence - SEQ ID NO: 8) for the indicated cells.

| | Myoblast | Myoblast | | |
|---|---|---|---|---|
| Variant # | (ab1190) | (ac16) | C2C12 | HEK293T |
| V1 | 2.41 | 1.67 | 0.73 | 1.24 |
| V5 | 2.74 | 1.51 | 1.37 | 1.23 |
| V6 | 2.86 | 1.09 | 0.93 | 1.32 |
| V7 | −1.08 | 0.11 | −4.21 | −0.47 |
| V8 | 2.34 | 1.89 | 0.66 | 0.89 |
| V9 | 2.62 | 1.81 | 0.29 | 1.32 |
| V10 | 3.63 | 1.79 | 0.51 | 1.53 |
| V11 | 0.43 | 0.94 | −0.57 | 0.51 |
| V12 | 2.55 | 0.42 | −2.22 | 1.05 |
| V13 | 1.20 | 0.79 | 1.11 | 0.67 |
| V14 | 2.99 | 1.38 | 0.33 | 1.37 |
| V15 | 1.51 | 0.29 | 0.48 | 0.82 |
| V16 | 1.00 | −0.24 | −6.00 | −0.07 |
| V17 | 0.31 | −1.03 | −0.64 | 0.16 |
| V18 | 0.67 | 0.58 | −0.17 | 0.59 |
| V19 | 2.75 | 2.32 | −0.01 | 1.52 |
| V20 | 3.61 | 1.60 | 0.88 | 1.20 |
| V21 | 2.80 | 1.18 | 0.52 | 1.13 |
| V22 | 1.48 | 0.75 | −3.50 | 0.02 |
| V23 | −2.96 | −0.03 | 0.40 | 0.54 |
| V24 | 1.37 | 0.48 | −1.21 | 0.56 |
| V25 | −1.46 | −2.34 | −1.69 | 0.34 |
| V26 | 3.37 | 1.25 | −0.68 | 0.98 |
| V27 | 1.31 | 0.43 | 1.87 | 0.54 |
| V28 | 2.80 | −1.33 | 0.32 | −0.20 |
| V29 | 2.88 | 1.05 | 0.27 | 0.48 |
| V30 | 0.34 | 1.71 | −1.47 | 1.06 |
| V31 | −1.10 | 0.34 | −3.50 | 0.13 |
| V32 | 4.41 | 1.55 | 1.03 | 1.54 |
| V33 | 0.17 | 0.43 | 0.73 | 0.47 |
| V34 | 2.00 | 1.24 | −0.25 | 1.57 |
| V35 | −0.70 | 0.17 | −0.52 | 0.19 |
| V36 | 0.34 | 0.55 | −1.06 | −0.19 |
| V37 | 0.49 | 1.26 | −0.39 | 0.80 |
| V38 | −6.00 | −0.59 | −6.00 | 0.33 |
| V39 | 3.33 | 0.27 | 1.22 | 1.70 |
| V40 | −1.30 | −0.60 | −1.11 | 0.19 |
| V41 | 2.05 | 0.41 | 0.94 | 1.10 |
| V42 | 0.95 | 0.18 | −0.75 | 0.31 |
| V43 | 2.00 | −2.69 | −0.30 | −0.33 |
| V44 | 3.57 | 1.73 | 2.16 | 1.94 |
| V45 | 0.61 | 0.91 | −1.43 | 0.38 |
| V46 | 0.86 | −0.15 | 1.24 | −0.38 |
| V47 | −1.81 | 0.48 | −0.63 | 0.44 |
| V48 | 0.72 | 0.90 | −1.55 | 0.36 |
| V49 | 3.58 | 1.80 | 1.30 | 1.79 |
| V50 | 4.04 | 1.94 | 0.12 | 1.29 |
| V51 | −1.07 | 0.05 | −1.30 | 0.53 |
| V52 | 1.21 | 0.91 | −0.19 | 0.94 |
| V53 | 1.87 | 1.55 | 0.43 | 0.83 |
| V54 | 4.00 | 1.10 | 1.51 | 1.93 |
| V55 | −0.84 | −0.14 | −1.66 | 0.15 |
| V56 | 1.11 | 1.07 | 2.36 | 0.74 |
| V57 | −1.37 | −1.67 | −6.00 | −0.09 |
| V58 | 0.70 | 1.40 | 2.55 | 1.44 |
| V59 | −2.60 | −6.00 | −6.00 | −0.38 |
| V60 | −2.36 | 0.15 | −1.36 | 0.04 |
| V61 | −0.39 | 0.57 | −0.36 | 0.43 |
| V62 | −6.00 | −0.26 | −1.20 | −0.13 |
| V63 | 0.53 | 1.45 | 0.75 | 0.88 |
| V64 | 1.97 | −0.37 | 0.02 | 0.79 |
| V65 | −6.00 | −1.21 | 6.00 | −0.20 |
| V66 | −1.65 | −0.21 | −2.47 | 0.01 |
| V67 | 2.94 | 1.22 | −1.46 | 1.44 |
| V68 | 0.72 | 0.18 | −0.09 | 1.13 |
| V69 | −0.29 | 0.19 | 1.32 | 0.27 |
| V70 | 3.37 | 1.36 | 0.24 | 0.27 |
| V71 | 2.78 | 1.81 | 0.00 | 1.30 |
| V72 | −1.30 | −1.45 | −1.70 | −1.31 |
| V73 | 1.96 | 0.95 | 0.34 | 1.06 |
| V74 | 2.84 | 0.29 | −0.51 | 0.76 |
| V75 | −6.00 | 0.44 | −2.96 | −0.47 |
| V76 | 0.85 | 0.97 | 0.50 | 0.87 |
| V77 | 1.97 | 0.55 | 0.15 | 1.34 |
| V78 | 0.98 | −1.49 | 0.31 | 0.48 |
| V79 | 1.77 | 0.21 | 0.28 | 1.61 |
| V80 | 1.15 | −1.32 | 0.33 | 0.69 |
| V81 | −0.93 | 0.36 | −1.16 | −0.05 |
| V82 | −6.00 | 0.81 | −3.55 | 0.20 |
| V83 | −0.26 | −2.73 | −2.08 | −1.54 |
| V84 | 1.78 | 0.48 | −1.32 | 0.85 |
| V85 | −1.73 | −1.29 | −1.22 | 0.11 |
| V86 | 1.24 | −1.02 | −0.69 | 0.32 |
| V87 | 0.03 | 1.62 | −3.60 | −0.09 |
| V88 | 2.18 | 0.77 | 0.70 | 0.39 |
| V89 | −0.45 | −1.52 | −1.94 | 0.14 |
| V90 | 1.50 | −0.38 | 1.06 | 1.06 |
| V91 | −6.00 | −1.57 | −3.09 | −0.65 |
| V92 | −6.00 | 0.76 | −2.98 | 0.03 |
| V93 | 2.07 | 0.19 | 0.26 | 0.78 |
| V94 | −0.35 | −1.23 | −2.49 | −0.10 |
| V95 | 0.64 | −0.65 | −6.00 | 0.27 |
| V96 | −1.18 | 2.64 | −1.99 | −0.26 |
| V97 | −0.21 | −1.09 | 0.43 | 0.10 |
| V98 | 0.37 | −3.25 | 0.51 | 0.00 |
| V99 | −1.31 | −1.02 | −3.13 | −0.10 |
| V100 | 2.63 | 0.87 | 1.38 | 0.84 |
| V101 | 0.99 | −2.22 | 6.00 | −0.08 |
| V102 | 0.10 | 0.44 | −2.30 | 0.46 |
| V103 | −0.43 | −2.89 | −6.00 | 0.44 |
| V104 | −6.00 | −2.45 | −6.00 | −0.66 |
| V105 | −1.13 | −1.60 | −3.53 | −0.16 |
| V106 | 3.79 | 0.86 | 2.05 | 1.70 |
| V107 | −0.13 | 0.80 | 0.21 | 0.59 |
| V108 | 2.77 | 1.28 | 0.41 | 1.76 |
| V109 | −0.49 | 0.95 | 0.43 | 1.07 |
| V110 | 2.76 | 0.96 | −0.15 | −0.20 |
| V111 | −1.81 | 0.47 | 6.00 | 0.25 |
| V112 | 2.48 | 0.20 | 0.82 | 0.83 |
| V113 | 3.43 | 0.08 | 0.14 | 1.29 |
| V114 | −2.29 | −1.59 | −1.52 | −0.76 |
| V115 | −0.02 | −0.22 | −0.35 | 0.05 |
| V116 | −6.00 | 0.06 | −0.14 | 0.19 |
| V117 | −0.92 | −0.39 | −6.00 | 0.09 |
| V118 | −6.00 | −1.06 | 0.59 | −0.19 |
| V119 | −0.10 | 0.34 | −0.10 | 0.72 |
| V120 | 0.22 | −0.13 | 0.17 | 0.68 |
| V121 | 0.29 | 0.87 | 0.28 | 1.28 |
| V122 | −0.34 | −1.48 | −2.74 | −0.03 |
| V123 | −6.00 | 0.01 | −6.00 | −0.12 |
| V124 | 3.58 | 0.22 | 0.66 | 1.49 |
| V125 | −2.16 | −1.72 | −0.66 | −0.04 |
| V126 | −2.96 | −0.52 | −2.78 | −0.57 |
| V127 | −1.13 | −0.79 | −0.95 | 0.54 |
| V128 | −0.04 | −2.51 | −0.86 | −0.01 |
| V129 | 1.68 | 1.06 | 0.35 | 1.02 |
| V130 | −6.00 | −0.89 | −6.00 | 0.04 |
| V131 | −1.74 | −0.30 | −6.00 | −0.37 |
| V132 | 0.20 | −1.23 | −0.96 | 0.79 |
| V133 | 1.92 | 1.03 | −0.48 | 0.83 |
| V134 | 1.21 | −0.34 | −1.85 | 0.07 |
| V135 | −1.17 | −0.78 | −1.98 | −0.65 |
| V136 | 1.83 | 0.95 | −1.31 | 1.13 |
| V137 | −6.00 | −1.26 | −0.78 | −0.01 |
| V138 | −1.97 | −1.26 | −2.78 | −0.54 |
| V139 | −0.42 | 1.46 | −1.23 | 0.36 |
| V140 | 2.18 | −6.00 | −6.00 | 0.15 |
| V141 | −1.68 | −0.56 | −0.68 | 0.70 |
| V142 | −1.65 | −1.54 | −6.00 | −1.40 |
| V143 | −0.25 | −1.13 | −2.65 | −0.36 |
| V144 | −6.00 | −2.09 | −0.03 | 0.31 |
| V145 | −1.70 | −1.58 | −2.51 | −0.08 |
| V146 | −1.20 | −0.49 | 0.31 | −0.53 |
| V147 | −0.96 | −0.26 | −3.36 | 0.39 |
| V148 | −1.79 | −6.00 | −6.00 | −0.54 |
| V149 | −0.83 | −0.90 | −1.32 | −0.12 |
| V150 | −6.00 | −2.31 | −6.00 | −0.18 |
| Known RGD- | 0.05 | 0.35 | 0.29 | −0.36 |
| insertion | | | | |
| containing capsid | | | | |
| variant | | | | |

Production efficiencies of variant capsids V1 and V5-V150 (having subcombinations of mutations of V1), along with a known RGD-insertion containing capsid variant, relative to V1 are summarized in Table 8A. The changes in biodistribution and transduction in various tissues in cynomolgus monkeys relative to V1 are summarized in Table 8B (biodistribution relative to V1) and Table 80 (transduction relative to V1) The changes in biodistribution and transduction in various mouse tissues relative to V1 are summarized in Table 8D (biodistribution relative to V1) and Table 8E (transduction relative to V1). The changes in in vitro transduction in various cells relative to V1 are summarized in Table 8F. All values are reported as the log 2 fold change relative to V1.

TABLE 8A
Production efficiency of variant virus particles relative to
production efficiency of V1 virus in HEK293 cells in vitro.

| Variant # | Fold change relative to wtAAV9 |
|---|---|
| V1 | 0.00 |
| V5 | 0.53 |
| V6 | 0.36 |
| V7 | 0.30 |
| V8 | 0.79 |
| V9 | 0.80 |
| V10 | 0.07 |
| V11 | 0.10 |
| V12 | 0.07 |
| V13 | 0.38 |
| V14 | 0.43 |
| V15 | 0.17 |
| V16 | 0.03 |
| V17 | 0.54 |
| V18 | 0.49 |
| V19 | 0.04 |
| V20 | 0.71 |
| V21 | 0.00 |
| V22 | 0.29 |
| V23 | 1.41 |
| V24 | 0.23 |
| V25 | 0.11 |
| V26 | 0.21 |
| V27 | 0.03 |
| V28 | 0.47 |
| V29 | 1.08 |
| V30 | −0.18 |
| V31 | 0.56 |
| V32 | 0.21 |
| V33 | 0.65 |
| V34 | 1.03 |
| V35 | 0.46 |
| V36 | −0.21 |
| V37 | 0.65 |
| V38 | 0.00 |
| V39 | 0.34 |
| V40 | 0.29 |
| V41 | 1.17 |
| V42 | 0.39 |
| V43 | 0.13 |
| V44 | 0.91 |
| V45 | −0.71 |
| V46 | 0.00 |
| V47 | 0.50 |
| V48 | 0.72 |
| V49 | 0.85 |
| V50 | 0.94 |
| V51 | 0.67 |
| V52 | 0.51 |
| V53 | 0.17 |
| V54 | 1.06 |
| V55 | −0.26 |
| V56 | 0.41 |
| V57 | 0.66 |
| V58 | 0.03 |
| V59 | 0.03 |
| V60 | 0.65 |
| V61 | 0.36 |
| V62 | 0.59 |
| V63 | 0.72 |
| V64 | 0.41 |
| V65 | −0.53 |
| V66 | −0.14 |
| V67 | 0.32 |
| V68 | 0.64 |
| V69 | 0.25 |
| V70 | 0.39 |
| V71 | 1.39 |
| V72 | 0.45 |
| V73 | 1.11 |
| V74 | −0.63 |
| V75 | 0.56 |
| V76 | 0.76 |
| V77 | 0.82 |
| V78 | 0.99 |
| V79 | 0.09 |
| V80 | −0.78 |
| V81 | −0.13 |
| V82 | 0.40 |
| V83 | 0.71 |
| V84 | 1.24 |
| V85 | −0.40 |
| V86 | 0.18 |
| V87 | 0.04 |
| V88 | 0.54 |
| V89 | 0.59 |
| V90 | 0.37 |
| V91 | −0.35 |
| V92 | −0.05 |
| V93 | 0.47 |
| V94 | 0.36 |
| V95 | 0.79 |
| V96 | 0.19 |
| V97 | 0.42 |
| V98 | 0.03 |
| V99 | 1.19 |
| V100 | 0.50 |
| V101 | −0.26 |
| V102 | 0.04 |
| V103 | −0.39 |
| V104 | 0.79 |
| V105 | 0.40 |
| V106 | 0.38 |
| V107 | 0.54 |
| V108 | 0.57 |
| V109 | 0.48 |
| V110 | −0.41 |
| V111 | −0.45 |
| V112 | 0.62 |
| V113 | 0.85 |
| V114 | 0.23 |
| V115 | 0.23 |
| V116 | 0.95 |
| V117 | 0.53 |
| V118 | 0.66 |
| V119 | −0.23 |
| V120 | 0.46 |
| V121 | 0.59 |
| V122 | 0.02 |
| V123 | −0.18 |
| V124 | 0.58 |
| V125 | 0.31 |
| V126 | 0.67 |
| V127 | 0.01 |
| V128 | −0.08 |
| V129 | 0.62 |
| V130 | −0.39 |
| V131 | −0.45 |
| V132 | 0.62 |
| V133 | −0.10 |
| V134 | 1.13 |
| V135 | 0.25 |
| V136 | 1.42 |
| V137 | −0.19 |
| V138 | 0.22 |
| V139 | 0.36 |
| V140 | −0.64 |
| V141 | 0.39 |
| V142 | −0.82 |
| V143 | −0.09 |
| V144 | −0.64 |
| V145 | −0.22 |
| V146 | 0.51 |
| V147 | 0.61 |
| V148 | 0.20 |
| V149 | 0.44 |
| V150 | −0.40 |
| Known RGD-insertion containing | −0.16 |
| capsid variant | |

TABLE 8B
Fold change in biodistribution of virus particles comprising the indicated
AAV capsid variant polypeptide in cynomolgus monkey tissues, relative
to the biodistribution of virus particles comprising V1 (amino acid
sequence - SEQ ID NO: 12; nucleotide sequence - SEQ ID NO: 13) for
the indicated tissues. “Aggregated” in relation to a tissue
is an average across all measured areas in the specified organ.

| Variant # | Heart | Liver | Muscle (aggregated) |
|---|---|---|---|
| V1 | 0.00 | 0.00 | 0.00 |
| V5 | 0.17 | 0.17 | 0.21 |
| V6 | 0.05 | 0.69 | 0.81 |
| V7 | −1.23 | 0.23 | −0.06 |
| V8 | 0.05 | −0.04 | 0.01 |
| V9 | 0.01 | −0.27 | 0.12 |
| V10 | −1.85 | −1.27 | −1.36 |
| V11 | −0.17 | 0.78 | 0.15 |
| V12 | −0.38 | 0.59 | 0.23 |
| V13 | −0.63 | 0.82 | −0.12 |
| V14 | −0.47 | 0.43 | 0.26 |
| V15 | −0.36 | 1.02 | 0.74 |
| V16 | −1.31 | −0.10 | −0.22 |
| V17 | −0.88 | −0.23 | −0.38 |
| V18 | −0.74 | 0.89 | −0.22 |
| V19 | 0.18 | 0.01 | −0.31 |
| V20 | −0.04 | 0.72 | 0.56 |
| V21 | −0.18 | −0.27 | 0.19 |
| V22 | 0.36 | 0.57 | 0.24 |
| V23 | 0.91 | 0.53 | −0.12 |
| V24 | −0.50 | 0.58 | 0.00 |
| V25 | −1.13 | −0.34 | −1.04 |
| V26 | −0.29 | −0.18 | −0.11 |
| V27 | −0.35 | 0.76 | 0.13 |
| V28 | −0.05 | 0.90 | 0.60 |
| V29 | 0.29 | 0.79 | 0.17 |
| V30 | 0.80 | 0.92 | 0.51 |
| V31 | −1.04 | 0.02 | −0.32 |
| V32 | −0.09 | 0.97 | 0.62 |
| V33 | 0.33 | 0.65 | −0.19 |
| V34 | −1.08 | 0.53 | −0.31 |
| V35 | −1.24 | −0.52 | −0.78 |
| V36 | −0.83 | 0.00 | 0.15 |
| V37 | −0.57 | 0.62 | 0.21 |
| V38 | −1.10 | −0.65 | −1.55 |
| V39 | −0.25 | 0.79 | 0.58 |
| V40 | −1.21 | −0.17 | −0.48 |
| V41 | −0.08 | 1.05 | 0.75 |
| V42 | 0.28 | 0.90 | 0.41 |
| V43 | −0.31 | 0.60 | 0.06 |
| V44 | 0.32 | 0.72 | 0.18 |
| V45 | −0.20 | 0.79 | 0.09 |
| V46 | −1.96 | 0.77 | −1.09 |
| V47 | −0.68 | 0.43 | 0.17 |
| V48 | −0.12 | 0.80 | 0.98 |
| V49 | −0.02 | 0.93 | 0.38 |
| V50 | −2.64 | −0.45 | −0.76 |
| V51 | −1.16 | −0.34 | −0.97 |
| V52 | −0.19 | 1.11 | 1.21 |
| V53 | −0.09 | 0.72 | 0.57 |
| V54 | 0.09 | 1.12 | 0.40 |
| V55 | −7.21 | −5.19 | −6.30 |
| V56 | 0.05 | 0.79 | 0.06 |
| V57 | −0.98 | 0.05 | 0.68 |
| V58 | 0.23 | 0.97 | 0.50 |
| V59 | −1.01 | 0.08 | −1.24 |
| V60 | −1.14 | −0.41 | 0.77 |
| V61 | 0.79 | 0.50 | 0.15 |
| V62 | −1.08 | −0.03 | −0.72 |
| V63 | −0.72 | 0.43 | −0.40 |
| V64 | 0.28 | 0.54 | 0.20 |
| V65 | −1.29 | 0.03 | 0.70 |
| V66 | −1.25 | −0.41 | 0.84 |
| V67 | −0.07 | 0.42 | 0.13 |
| V68 | −0.81 | 0.28 | 0.04 |
| V69 | 0.71 | 0.48 | 0.13 |
| V70 | −0.05 | 0.85 | 0.38 |
| V71 | −1.40 | 0.03 | −1.26 |
| V72 | −1.23 | −0.37 | 0.28 |
| V73 | 0.05 | 0.97 | 0.62 |
| V74 | −0.27 | 1.00 | 0.44 |
| V75 | −1.35 | 0.06 | −1.13 |
| V76 | −0.68 | 0.78 | 0.16 |
| V77 | −0.20 | 0.69 | 0.23 |
| V78 | −0.16 | 0.83 | 0.34 |
| V79 | 0.30 | 1.11 | 0.02 |
| V80 | −0.22 | 0.65 | 0.11 |
| V81 | −0.45 | 0.72 | 0.27 |
| V82 | −1.24 | −0.86 | 0.68 |
| V83 | −0.88 | 0.18 | 0.11 |
| V84 | −1.30 | −0.15 | 0.73 |
| V85 | −1.09 | −0.03 | 0.48 |
| V86 | −1.60 | 0.92 | −0.71 |
| V87 | −0.72 | 0.30 | 0.13 |
| V88 | 0.30 | 0.72 | 0.42 |
| V89 | −0.70 | 0.66 | −0.17 |
| V90 | −0.20 | 1.07 | 0.11 |
| V91 | −1.24 | 0.29 | 0.60 |
| V92 | −1.31 | −0.01 | −0.06 |
| V93 | −0.43 | 0.86 | 0.12 |
| V94 | −0.81 | 0.50 | 0.02 |
| V95 | 0.97 | 0.63 | −0.37 |
| V96 | −1.03 | 0.57 | 0.27 |
| V97 | −1.79 | −0.24 | −0.60 |
| V98 | −0.85 | 0.55 | 0.12 |
| V99 | 0.55 | 0.41 | 0.21 |
| V100 | −0.16 | 0.93 | 0.42 |
| V101 | −1.74 | 0.10 | 1.19 |
| V102 | 0.58 | 0.12 | 0.68 |
| V103 | −1.02 | −0.21 | 0.74 |
| V104 | −1.29 | 0.21 | 0.70 |
| V105 | −1.15 | −0.26 | −0.69 |
| V106 | −0.17 | 1.04 | 0.41 |
| V107 | −0.76 | 0.24 | −0.20 |
| V108 | 0.02 | 0.96 | 0.34 |
| V109 | −0.12 | 1.00 | 0.50 |
| V110 | −0.08 | 0.61 | 0.00 |
| V111 | −1.27 | −0.33 | −0.53 |
| V112 | −0.33 | 1.01 | 0.53 |
| V113 | −0.09 | 0.99 | 0.29 |
| V114 | −0.84 | 0.44 | 0.02 |
| V115 | −0.99 | 0.57 | 0.08 |
| V116 | −0.51 | 0.74 | 0.06 |
| V117 | −0.94 | −0.15 | −0.79 |
| V118 | −1.89 | −0.82 | −0.85 |
| V119 | −1.61 | 0.04 | −1.17 |
| V120 | −0.85 | 0.28 | 0.86 |
| V121 | −0.54 | 0.66 | −0.35 |
| V122 | −1.44 | 0.39 | −0.87 |
| V123 | 1.22 | −0.51 | −0.40 |
| V124 | −0.12 | 0.96 | 0.37 |
| V125 | −0.86 | 0.55 | −0.17 |
| V126 | −1.22 | −0.36 | −0.81 |
| V127 | −0.80 | 0.50 | 0.09 |
| V128 | −0.90 | 0.72 | −0.06 |
| V129 | −0.08 | 1.14 | 0.43 |
| V130 | −1.39 | 0.16 | 0.30 |
| V131 | 1.27 | 0.47 | −0.85 |
| V132 | −1.28 | −0.20 | −0.66 |
| V133 | −1.27 | 0.63 | −0.59 |
| V134 | −0.80 | 0.30 | −0.14 |
| V135 | −1.10 | 0.79 | −0.07 |
| V136 | −0.11 | 1.19 | 0.00 |
| V137 | −0.61 | 0.51 | −0.40 |
| V138 | −0.95 | 0.30 | −0.44 |
| V139 | −0.55 | 0.53 | −0.05 |
| V140 | −1.20 | 0.73 | −0.63 |
| V141 | −1.01 | 0.41 | 0.16 |
| V142 | −1.84 | 0.23 | 0.20 |
| V143 | −1.10 | 0.87 | −0.24 |
| V144 | −1.21 | 0.96 | 0.23 |
| V145 | −0.88 | 0.81 | −0.01 |
| V146 | −1.11 | 1.00 | −0.52 |
| V147 | −0.68 | 0.71 | −0.28 |
| V148 | −1.28 | 0.76 | −0.40 |
| V149 | −1.20 | 0.87 | −0.01 |
| V150 | −2.57 | −0.25 | −0.60 |
| Known RGD- | −1.86 | −1.40 | −1.18 |
| insertion | | | |
| containing capsid | | | |
| variant | | | |

TABLE 8C
Fold change in transduction of virus particles comprising the indicated
AAV capsid variant polypeptide in cynomolgus monkey tissues, relative
to the transduction of virus particles comprising V1 (amino acid
sequence - SEQ ID NO: 12; nucleotide sequence - SEQ ID NO: 13)
for the indicated tissues. “Aggregated” in relation to a
tissue is an average across all measured areas in the specified organ.

| Variant # | Heart | Muscle (aggregated) |
|---|---|---|
| V1 | 0.00 | 0.00 |
| V5 | 0.01 | −0.24 |
| V6 | −0.49 | −0.35 |
| V7 | 0.81 | −0.92 |
| V8 | 0.18 | −0.11 |
| V9 | −0.09 | −0.05 |
| V10 | −2.57 | −1.78 |
| V11 | 0.01 | 0.10 |
| V12 | −0.70 | 0.06 |
| V13 | −0.69 | −1.50 |
| V14 | −0.40 | 0.16 |
| V15 | 0.01 | 0.01 |
| V16 | −2.37 | 1.09 |
| V17 | −1.06 | −0.71 |
| V18 | −1.12 | −1.61 |
| V19 | 0.24 | 0.27 |
| V20 | −0.35 | 0.09 |
| V21 | 0.03 | 0.02 |
| V22 | −1.00 | −0.60 |
| V23 | −0.80 | 0.51 |
| V24 | 0.13 | 0.17 |
| V25 | −0.87 | −0.66 |
| V26 | −0.26 | −0.16 |
| V27 | −0.17 | −0.21 |
| V28 | −2.05 | −1.29 |
| V29 | 0.28 | 0.49 |
| V30 | −0.08 | −1.48 |
| V31 | −0.73 | −1.01 |
| V32 | 0.05 | 0.46 |
| V33 | −0.26 | −0.18 |
| V34 | −0.50 | −1.52 |
| V35 | −0.41 | −0.41 |
| V36 | −1.09 | −1.98 |
| V37 | −0.61 | −1.25 |
| V38 | −0.64 | −0.91 |
| V39 | −0.13 | 0.18 |
| V40 | −0.27 | −0.72 |
| V41 | −0.37 | 0.26 |
| V42 | −0.60 | −0.06 |
| V43 | −0.97 | −0.65 |
| V44 | 0.29 | 0.71 |
| V45 | −0.34 | 0.19 |
| V46 | −4.07 | −3.37 |
| V47 | 0.02 | −0.40 |
| V48 | −0.35 | 0.17 |
| V49 | −0.03 | 0.72 |
| V50 | −2.43 | −3.01 |
| V51 | −0.16 | −0.71 |
| V52 | −1.44 | 0.02 |
| V53 | 0.00 | 0.20 |
| V54 | 0.30 | 0.39 |
| V55 | −7.14 | −7.95 |
| V56 | −0.04 | 0.44 |
| V57 | −1.15 | −0.70 |
| V58 | −0.16 | 0.56 |
| V59 | −0.26 | −1.58 |
| V60 | 1.35 | −0.76 |
| V61 | −1.02 | −0.63 |
| V62 | −0.73 | −0.51 |
| V63 | −0.44 | −1.48 |
| V64 | −0.68 | 0.13 |
| V65 | −0.85 | −1.72 |
| V66 | −0.77 | −0.98 |
| V67 | −0.09 | 0.11 |
| V68 | −0.65 | −0.29 |
| V69 | −0.95 | −0.66 |
| V70 | −0.60 | −0.17 |
| V71 | −0.94 | −1.47 |
| V72 | −2.01 | −1.32 |
| V73 | 0.09 | 0.11 |
| V74 | −0.50 | 0.03 |
| V75 | −1.40 | −1.13 |
| V76 | −0.57 | −1.24 |
| V77 | 0.40 | 0.66 |
| V78 | −0.30 | 0.40 |
| V79 | 0.52 | 0.91 |
| V80 | 0.32 | 0.76 |
| V81 | −0.37 | −0.66 |
| V82 | −0.20 | −0.74 |
| V83 | −2.59 | −2.42 |
| V84 | −0.73 | −1.88 |
| V85 | −0.47 | −1.30 |
| V86 | −2.18 | −2.85 |
| V87 | −0.93 | −0.66 |
| V88 | −0.34 | 0.20 |
| V89 | −0.78 | −0.83 |
| V90 | −0.41 | 0.19 |
| V91 | −0.72 | −1.35 |
| V92 | −1.56 | −1.80 |
| V93 | 0.93 | 0.44 |
| V94 | −1.36 | −0.63 |
| V95 | −0.88 | −1.17 |
| V96 | −0.73 | −1.39 |
| V97 | −0.72 | −0.52 |
| V98 | −0.64 | −0.93 |
| V99 | −0.29 | −0.46 |
| V100 | −0.20 | 0.49 |
| V101 | −0.77 | −0.83 |
| V102 | −2.34 | −0.82 |
| V103 | −0.76 | −1.26 |
| V104 | −1.76 | −1.14 |
| V105 | −1.04 | −1.30 |
| V106 | −0.04 | 0.31 |
| V107 | −0.87 | −0.41 |
| V108 | 0.30 | 0.56 |
| V109 | 0.59 | 0.37 |
| V110 | −0.53 | −0.67 |
| V111 | −0.32 | −0.64 |
| V112 | −0.42 | −0.01 |
| V113 | −0.36 | 0.60 |
| V114 | −0.78 | −1.01 |
| V115 | −1.17 | −0.80 |
| V116 | −1.00 | −0.35 |
| V117 | −0.76 | −1.35 |
| V118 | −1.13 | −1.42 |
| V119 | −0.80 | −0.69 |
| V120 | −0.43 | −0.44 |
| V121 | 0.59 | 0.68 |
| V122 | −1.12 | −0.96 |
| V123 | −0.89 | −1.08 |
| V124 | 0.74 | 0.75 |
| V125 | −0.92 | −1.43 |
| V126 | −1.00 | −1.21 |
| V127 | −0.74 | −0.51 |
| V128 | −0.94 | −1.08 |
| V129 | 0.06 | 0.21 |
| V130 | −0.69 | −0.69 |
| V131 | −2.93 | −2.04 |
| V132 | −0.85 | 0.45 |
| V133 | −0.87 | −0.94 |
| V134 | −1.22 | −0.82 |
| V135 | −1.46 | −2.18 |
| V136 | −0.36 | 0.32 |
| V137 | −0.81 | −0.78 |
| V138 | −2.59 | −1.38 |
| V139 | −0.25 | −0.44 |
| V140 | −1.66 | −3.16 |
| V141 | −1.17 | −0.62 |
| V142 | −0.86 | −2.10 |
| V143 | 0.40 | −2.15 |
| V144 | 0.24 | −1.11 |
| V145 | −0.63 | −2.63 |
| V146 | −2.39 | −1.84 |
| V147 | 0.38 | −0.94 |
| V148 | −1.02 | −1.55 |
| V149 | −0.77 | −1.90 |
| V150 | −1.80 | −2.45 |
| Known RGD-insertion | −2.55 | −1.72 |
| containing capsid variant | | |

TABLE 8D
Fold change in biodistribution of virus particles comprising the
indicated AAV capsid variant polypeptide in mouse tissues, relative
to the biodistribution of virus particles comprising V1 (amino acid
sequence - SEQ ID NO: 12; nucleotide sequence - SEQ ID NO: 13) for
the indicated tissues. “Aggregated” in relation to a tissue
is an average across all measured areas in the specified organ.

| Variant # | Heart | Liver | Muscle (aggregated) |
|---|---|---|---|
| V1 | 0.00 | 0.00 | 0.00 |
| V5 | 0.24 | 0.24 | −1.00 |
| V6 | −0.37 | 1.53 | 0.88 |
| V7 | −0.25 | −0.54 | 0.91 |
| V8 | −0.20 | −0.77 | −1.12 |
| V9 | 0.61 | 0.27 | −0.14 |
| V10 | −1.28 | −2.60 | −4.53 |
| V11 | 0.62 | 1.96 | 1.39 |
| V12 | 0.79 | 1.53 | 0.64 |
| V13 | −0.16 | 1.40 | 0.70 |
| V14 | 0.08 | 1.22 | 0.16 |
| V15 | 0.09 | 2.24 | 0.92 |
| V16 | 0.44 | 0.39 | −0.96 |
| V17 | 0.60 | −0.85 | −0.34 |
| V18 | −0.18 | 1.48 | 0.72 |
| V19 | 0.08 | 0.21 | −1.12 |
| V20 | 0.50 | 1.50 | 0.81 |
| V21 | −0.31 | 1.29 | 0.01 |
| V22 | 0.59 | 1.88 | 0.70 |
| V23 | 0.39 | 0.76 | 0.71 |
| V24 | 0.87 | 0.89 | 1.66 |
| V25 | −0.72 | −0.57 | −0.62 |
| V26 | 0.23 | −0.12 | 0.05 |
| V27 | 0.65 | 1.82 | 0.45 |
| V28 | −0.40 | 1.33 | 0.63 |
| V29 | 0.07 | 1.70 | 0.81 |
| V30 | −1.34 | 0.18 | −0.22 |
| V31 | −0.68 | 0.05 | 0.64 |
| V32 | −0.13 | 2.24 | 1.64 |
| V33 | 0.30 | 1.48 | 0.79 |
| V34 | −0.85 | −0.10 | −1.16 |
| V35 | −0.47 | −0.93 | −0.71 |
| V36 | −0.62 | 0.21 | 2.33 |
| V37 | −0.46 | 0.95 | 0.49 |
| V38 | 0.26 | −1.55 | 0.79 |
| V39 | −0.40 | 0.92 | 0.04 |
| V40 | −0.18 | −0.43 | −0.80 |
| V41 | 1.02 | 2.50 | 1.27 |
| V42 | 0.38 | 2.23 | 1.71 |
| V43 | 0.96 | 0.37 | 0.76 |
| V44 | 0.40 | 1.19 | −0.23 |
| V45 | 0.40 | 1.53 | −1.58 |
| V46 | 0.74 | 1.66 | 0.40 |
| V47 | −0.17 | 0.45 | 0.80 |
| V48 | 0.94 | 1.07 | 1.50 |
| V49 | 0.75 | 2.18 | 0.93 |
| V50 | −0.09 | 0.26 | −0.31 |
| V51 | 0.54 | −1.15 | 0.15 |
| V52 | 1.28 | 1.64 | −0.21 |
| V53 | 1.05 | 1.67 | −0.51 |
| V54 | 0.06 | 2.57 | 0.90 |
| V55 | 0.25 | 0.02 | 1.27 |
| V56 | 0.28 | 1.00 | 1.61 |
| V57 | −0.39 | 0.82 | −0.16 |
| V58 | 1.05 | 2.07 | 1.36 |
| V59 | 0.24 | 1.20 | 0.85 |
| V60 | −0.40 | −0.65 | 1.13 |
| V61 | −0.30 | 0.95 | −0.77 |
| V62 | −1.14 | 0.47 | 1.24 |
| V63 | −1.43 | 0.68 | 0.96 |
| V64 | 0.76 | 1.56 | 0.15 |
| V65 | −1.32 | 0.20 | −0.01 |
| V66 | 0.69 | −0.78 | −0.65 |
| V67 | 0.83 | −0.58 | 0.16 |
| V68 | −0.13 | 1.31 | 0.25 |
| V69 | 0.02 | 1.48 | 2.04 |
| V70 | 0.72 | 1.26 | 0.57 |
| V71 | −1.12 | 1.28 | 0.18 |
| V72 | 0.52 | −0.02 | 1.38 |
| V73 | 0.29 | 2.08 | 1.25 |
| V74 | 0.38 | 2.24 | 1.30 |
| V75 | −0.75 | 0.25 | −1.54 |
| V76 | −0.19 | 1.51 | 0.58 |
| V77 | 1.08 | 1.46 | 0.47 |
| V78 | −0.15 | 2.36 | 0.68 |
| V79 | −1.85 | 1.47 | 0.62 |
| V80 | 0.41 | 1.48 | −0.46 |
| V81 | −0.09 | 0.86 | 0.10 |
| V82 | −0.21 | −1.26 | −0.78 |
| V83 | 0.10 | 0.52 | 0.33 |
| V84 | −1.10 | −1.00 | −1.16 |
| V85 | −0.40 | 0.52 | 1.75 |
| V86 | −0.05 | 1.95 | 0.04 |
| V87 | 0.12 | 0.93 | 0.37 |
| V88 | 0.59 | 1.46 | 0.81 |
| V89 | 0.12 | 1.77 | 0.50 |
| V90 | 0.14 | 2.08 | 1.16 |
| V91 | −1.04 | 0.10 | 2.01 |
| V92 | 0.06 | −0.72 | 0.26 |
| V93 | −0.22 | 2.45 | 0.20 |
| V94 | −0.19 | 1.67 | 0.27 |
| V95 | −0.19 | 1.87 | −0.39 |
| V96 | 0.41 | 1.53 | 1.26 |
| V97 | 0.18 | −0.14 | 1.27 |
| V98 | 0.69 | 1.16 | 0.63 |
| V99 | −0.23 | 0.64 | 0.09 |
| V100 | 0.18 | 2.50 | 1.02 |
| V101 | −0.22 | 0.13 | 1.47 |
| V102 | 0.71 | 1.45 | −2.21 |
| V103 | 0.00 | 1.01 | 0.94 |
| V104 | −0.71 | −0.76 | 1.05 |
| V105 | −0.29 | 0.03 | 0.35 |
| V106 | 0.22 | 2.41 | 1.15 |
| V107 | 0.06 | 0.50 | 0.49 |
| V108 | 0.57 | 2.13 | 1.60 |
| V109 | 0.83 | 2.44 | 2.06 |
| V110 | −0.63 | 1.18 | −0.11 |
| V111 | 0.91 | 0.26 | 0.56 |
| V112 | 0.13 | 1.80 | 0.86 |
| V113 | 0.57 | 2.37 | 1.17 |
| V114 | 0.55 | 1.71 | 1.30 |
| V115 | 0.39 | 1.71 | 0.94 |
| V116 | 0.24 | 1.78 | 0.13 |
| V117 | −0.61 | −0.10 | −0.80 |
| V118 | −0.56 | −1.70 | −0.43 |
| V119 | −0.16 | 0.84 | −1.66 |
| V120 | −0.10 | 0.60 | 0.01 |
| V121 | 0.66 | 0.58 | −1.02 |
| V122 | 0.28 | −0.10 | −0.41 |
| V123 | 0.02 | −0.32 | 0.64 |
| V124 | 0.79 | 2.14 | 0.36 |
| V125 | 0.33 | 1.19 | 0.34 |
| V126 | −0.18 | −1.29 | 0.37 |
| V127 | 0.48 | 0.83 | 0.85 |
| V128 | 0.14 | 0.96 | 0.19 |
| V129 | 0.39 | 2.07 | 2.08 |
| V130 | 0.62 | 1.61 | 1.71 |
| V131 | −0.09 | 0.88 | 0.81 |
| V132 | −1.30 | −1.02 | −0.83 |
| V133 | 0.03 | 0.68 | 0.86 |
| V134 | 0.75 | 1.11 | −0.40 |
| V135 | −0.43 | 1.30 | 0.34 |
| V136 | 0.47 | 2.14 | 0.71 |
| V137 | 0.28 | 1.50 | 1.17 |
| V138 | 0.49 | 0.77 | −0.46 |
| V139 | 0.42 | 0.91 | 1.17 |
| V140 | −0.66 | 1.67 | −0.67 |
| V141 | 0.38 | 1.81 | 1.22 |
| V142 | 0.30 | 0.21 | 0.66 |
| V143 | 0.08 | 1.27 | 1.08 |
| V144 | −0.28 | 1.83 | 1.29 |
| V145 | 0.08 | 0.68 | 1.51 |
| V146 | −4.68 | 0.52 | 2.27 |
| V147 | 0.45 | 1.53 | 1.71 |
| V148 | −0.35 | 1.61 | 1.18 |
| V149 | 0.20 | 1.52 | −0.55 |
| V150 | −0.16 | −0.44 | 1.04 |
| Known RGD- | 1.79 | −0.10 | 2.47 |
| insertion | | | |
| containing capsid | | | |
| variant | | | |

TABLE 8E
Fold change in transduction of virus particles comprising
the indicated AAV capsid variant polypeptide in mouse
tissues, relative to the transduction of virus particles
comprising V1 (amino acid sequence - SEQ ID NO: 12; nucleotide
sequence - SEQ ID NO: 13) for the indicated tissues. “Aggregated”
in relation to a tissue is an average across all measured
areas in the specified organ.

| Variant # | Heart | Muscle (aggregated) |
|---|---|---|
| V1 | 0.00 | 0.00 |
| V5 | −0.13 | 1.04 |
| V6 | −0.79 | 0.32 |
| V7 | −0.74 | 0.31 |
| V8 | −0.16 | −0.45 |
| V9 | −0.40 | 0.39 |
| V10 | −2.22 | −0.14 |
| V11 | −0.49 | −0.50 |
| V12 | 0.70 | 1.04 |
| V13 | −0.74 | −0.24 |
| V14 | −0.32 | 0.13 |
| V15 | −0.09 | 0.66 |
| V16 | −0.45 | 0.02 |
| V17 | 0.19 | 0.15 |
| V18 | −0.48 | −0.38 |
| V19 | 0.14 | 1.46 |
| V20 | 0.57 | 0.70 |
| V21 | 0.34 | 0.44 |
| V22 | −0.20 | 0.16 |
| V23 | 0.36 | 0.16 |
| V24 | 0.95 | 0.84 |
| V25 | −0.16 | 0.40 |
| V26 | 0.16 | −0.07 |
| V27 | 0.24 | 0.00 |
| V28 | −1.53 | −1.36 |
| V29 | 0.58 | 0.87 |
| V30 | −0.28 | −0.61 |
| V31 | 0.21 | 0.55 |
| V32 | 0.96 | 0.18 |
| V33 | 0.42 | 1.19 |
| V34 | −0.96 | −1.13 |
| V35 | −0.55 | 0.05 |
| V36 | −0.56 | −1.13 |
| V37 | −0.50 | −0.24 |
| V38 | −0.44 | −0.86 |
| V39 | 0.33 | −0.97 |
| V40 | −0.14 | 0.15 |
| V41 | −0.05 | 0.76 |
| V42 | −0.54 | 0.09 |
| V43 | −0.28 | −0.47 |
| V44 | 0.44 | 0.55 |
| V45 | −0.19 | 0.98 |
| V46 | −1.22 | −0.05 |
| V47 | 0.00 | 0.23 |
| V48 | 0.20 | −0.19 |
| V49 | 0.91 | 0.19 |
| V50 | −0.39 | −0.85 |
| V51 | 0.44 | 0.65 |
| V52 | 0.72 | 0.54 |
| V53 | 0.83 | 1.16 |
| V54 | −0.19 | 1.04 |
| V55 | −0.81 | 0.14 |
| V56 | 1.13 | 1.58 |
| V57 | −0.50 | −0.46 |
| V58 | 0.05 | −0.33 |
| V59 | −0.57 | 0.17 |
| V60 | −0.21 | −0.48 |
| V61 | 0.18 | 0.08 |
| V62 | 0.76 | 0.31 |
| V63 | −0.28 | −0.16 |
| V64 | 0.66 | 0.29 |
| V65 | −0.22 | −0.86 |
| V66 | −0.24 | −0.84 |
| V67 | 0.65 | 0.34 |
| V68 | 0.07 | 0.62 |
| V69 | −0.17 | 0.24 |
| V70 | 0.34 | 1.09 |
| V71 | −1.42 | −1.03 |
| V72 | −0.76 | −0.40 |
| V73 | 0.05 | 0.33 |
| V74 | 0.23 | 0.40 |
| V75 | −0.39 | −0.44 |
| V76 | −0.22 | 0.06 |
| V77 | 0.92 | 1.27 |
| V78 | −0.11 | 0.77 |
| V79 | 0.48 | 0.42 |
| V80 | 0.59 | 0.64 |
| V81 | −0.35 | 0.47 |
| V82 | −0.40 | 0.02 |
| V83 | −1.94 | −0.77 |
| V84 | −0.88 | −0.75 |
| V85 | −0.40 | 0.09 |
| V86 | −0.31 | −1.63 |
| V87 | 0.55 | 0.43 |
| V88 | 0.32 | 0.29 |
| V89 | 0.19 | 0.10 |
| V90 | 0.82 | 0.70 |
| V91 | 0.18 | −0.61 |
| V92 | −0.04 | 0.23 |
| V93 | 0.18 | 1.35 |
| V94 | −0.56 | −0.11 |
| V95 | 0.94 | −1.89 |
| V96 | −0.25 | −0.04 |
| V97 | 0.33 | 0.72 |
| V98 | 0.01 | 0.41 |
| V99 | −0.14 | 0.05 |
| V100 | 0.45 | 0.27 |
| V101 | 0.75 | 1.39 |
| V102 | −0.05 | 0.65 |
| V103 | 0.43 | 0.55 |
| V104 | −0.54 | −0.07 |
| V105 | −0.02 | 0.60 |
| V106 | 0.84 | 1.42 |
| V107 | 0.32 | 0.49 |
| V108 | 0.74 | 0.97 |
| V109 | 0.19 | 0.40 |
| V110 | 0.08 | −0.92 |
| V111 | 0.46 | −0.01 |
| V112 | −0.07 | −0.81 |
| V113 | 0.59 | 1.56 |
| V114 | 0.04 | −0.09 |
| V115 | 0.01 | 0.04 |
| V116 | 0.72 | 1.64 |
| V117 | −0.29 | −0.49 |
| V118 | −1.08 | −1.61 |
| V119 | −0.16 | −0.64 |
| V120 | 0.23 | 0.60 |
| V121 | 0.43 | 1.04 |
| V122 | 0.13 | 0.57 |
| V123 | 1.06 | 0.81 |
| V124 | 0.63 | 0.83 |
| V125 | −0.43 | −0.65 |
| V126 | −0.22 | 0.65 |
| V127 | 0.91 | 0.60 |
| V128 | −1.15 | 0.31 |
| V129 | 0.45 | −0.57 |
| V130 | −0.59 | 0.00 |
| V131 | −0.60 | −2.24 |
| V132 | −1.11 | −1.06 |
| V133 | −1.54 | −0.01 |
| V134 | −0.23 | 0.18 |
| V135 | −1.10 | −0.66 |
| V136 | 0.16 | −0.19 |
| V137 | −0.29 | 0.56 |
| V138 | −0.26 | 0.22 |
| V139 | 0.35 | 0.26 |
| V140 | −0.36 | −0.45 |
| V141 | 0.14 | 0.68 |
| V142 | −0.34 | 0.74 |
| V143 | −0.43 | −1.77 |
| V144 | −0.72 | 1.19 |
| V145 | −0.04 | 0.51 |
| V146 | −1.54 | 0.63 |
| V147 | 0.24 | −0.46 |
| V148 | −1.69 | −0.16 |
| V149 | −0.17 | −0.08 |
| V150 | −0.76 | 0.08 |
| Known RGD-insertion | 1.63 | 2.76 |
| containing capsid variant | | |

TABLE 8F
Fold change in transduction of virus particles comprising
the indicated AAV capsid variant polypeptide in various cells
in vitro, relative to the transduction of virus particles
comprising V1 (amino acid sequence - SEQ ID NO: 12; nucleotide
sequence - SEQ ID NO: 13) for the indicated cells.

| | Myoblast | Myoblast | | |
|---|---|---|---|---|
| Variant # | (ab1190) | (ac16) | C2C12 | HEK293T |
| V1 | 0.00 | 0.00 | 0.00 | 0.00 |
| V5 | 0.33 | −0.16 | 0.64 | −0.01 |
| V6 | 0.45 | −0.59 | 0.20 | 0.08 |
| V7 | −3.49 | −1.56 | −4.94 | −1.71 |
| V8 | −0.07 | 0.21 | −0.07 | −0.35 |
| V9 | 0.21 | 0.14 | −0.44 | 0.08 |
| V10 | 1.22 | 0.11 | −0.21 | 0.29 |
| V11 | −1.97 | −0.73 | −1.30 | −0.73 |
| V12 | 0.14 | −1.26 | −2.94 | −0.20 |
| V13 | −1.20 | −0.89 | 0.38 | −0.57 |
| V14 | 0.58 | −0.30 | −0.40 | 0.12 |
| V15 | −0.89 | −1.39 | −0.25 | −0.43 |
| V16 | −1.40 | −1.91 | −6.73 | −1.31 |
| V17 | −2.10 | −2.70 | −1.37 | −1.08 |
| V18 | −1.74 | −1.09 | −0.89 | −0.66 |
| V19 | 0.34 | 0.65 | −0.74 | 0.28 |
| V20 | 1.20 | −0.08 | 0.15 | −0.04 |
| V21 | 0.39 | −0.50 | −0.21 | −0.11 |
| V22 | −0.93 | −0.92 | −4.23 | −1.22 |
| V23 | −5.36 | −1.70 | −0.33 | −0.70 |
| V24 | −1.04 | −1.19 | −1.94 | −0.68 |
| V25 | −3.87 | −4.02 | −2.42 | −0.91 |
| V26 | 0.96 | −0.42 | −1.41 | −0.26 |
| V27 | −1.10 | −1.25 | 1.15 | −0.71 |
| V28 | 0.39 | −3.01 | −0.41 | −1.44 |
| V29 | 0.47 | −0.62 | −0.45 | −0.76 |
| V30 | −2.07 | 0.04 | −2.20 | −0.19 |
| V31 | −3.51 | −1.33 | −4.23 | −1.11 |
| V32 | 2.00 | −0.12 | 0.30 | 0.29 |
| V33 | −2.24 | −1.25 | 0.01 | −0.77 |
| V34 | −0.40 | −0.43 | −0.97 | 0.32 |
| V35 | −3.11 | −1.84 | −1.25 | −1.06 |
| V36 | −2.07 | −2.22 | −1.79 | −1.43 |
| V37 | −1.92 | −0.41 | −1.12 | −0.44 |
| V38 | −8.41 | −2.26 | −6.73 | −0.91 |
| V39 | 0.92 | −1.40 | 0.49 | 0.46 |
| V40 | −3.71 | −2.27 | −1.84 | −1.05 |
| V41 | −0.36 | −1.27 | 0.21 | −0.15 |
| V42 | −1.45 | −1.49 | −1.48 | −0.93 |
| V43 | −0.41 | −4.36 | −1.03 | −1.57 |
| V44 | 1.16 | 0.06 | 1.44 | 0.70 |
| V45 | −3.02 | −2.58 | −2.16 | −0.87 |
| V46 | −3.26 | −1.83 | 0.51 | −1.62 |
| V47 | −4.22 | −1.20 | −1.36 | −0.80 |
| V48 | −1.69 | −0.77 | −2.28 | −0.89 |
| V49 | 1.18 | −0.13 | 0.57 | 0.55 |
| V50 | 1.63 | −0.26 | −0.61 | 0.05 |
| V51 | −3.48 | −1.63 | −2.03 | −0.71 |
| V52 | −1.20 | −0.76 | −0.92 | −0.31 |
| V53 | −0.54 | 0.13 | −0.30 | −0.42 |
| V54 | 1.59 | 0.58 | 0.78 | 0.69 |
| V55 | −3.25 | −1.81 | −2.39 | −1.10 |
| V56 | −1.30 | −0.60 | 1.63 | −0.51 |
| V57 | −3.78 | −3.34 | −6.73 | −1.33 |
| V58 | −1.71 | −0.27 | 1.83 | 0.20 |
| V59 | −5.01 | −7.67 | −6.73 | −1.62 |
| V60 | −4.76 | −1.52 | −2.09 | −1.20 |
| V61 | −2.80 | −1.10 | −1.09 | −0.82 |
| V62 | −8.41 | −1.94 | −1.92 | −1.37 |
| V63 | −1.88 | −0.23 | 0.03 | −0.36 |
| V64 | −0.44 | −2.04 | −0.71 | −0.45 |
| V65 | −8.41 | −2.88 | −6.73 | −1.44 |
| V66 | −4.06 | −1.89 | −3.20 | −1.23 |
| V67 | 0.53 | −0.45 | −2.19 | 0.20 |
| V68 | −1.68 | −1.49 | −0.82 | −0.12 |
| V69 | −2.70 | −1.48 | −2.05 | −0.97 |
| V70 | 0.96 | −0.31 | −0.49 | −0.97 |
| V71 | 0.37 | 0.14 | −0.73 | 0.06 |
| V72 | −3.71 | −3.12 | −2.43 | −2.56 |
| V73 | −0.45 | −0.73 | −0.38 | −0.18 |
| V74 | 0.44 | −1.38 | −1.24 | −0.49 |
| V75 | −8.41 | −2.11 | −3.68 | −1.72 |
| V76 | −1.56 | −0.71 | −0.23 | −0.37 |
| V77 | −0.44 | −1.13 | −0.57 | 0.10 |
| V78 | −1.43 | −3.16 | −0.41 | −0.77 |
| V79 | −0.64 | −1.46 | −0.45 | 0.37 |
| V80 | −1.26 | −2.99 | −0.40 | −0.55 |
| V81 | −3.34 | −1.31 | −1.89 | −1.29 |
| V82 | −8.41 | −2.48 | −4.28 | −1.04 |
| V83 | −2.67 | −4.40 | −2.80 | −2.78 |
| V84 | −0.62 | −1.20 | −2.04 | −0.40 |
| V85 | −4.14 | −2.96 | −1.95 | −1.14 |
| V86 | −1.17 | −2.69 | −1.42 | −0.93 |
| V87 | −2.38 | −0.05 | −4.32 | −1.33 |
| V88 | −0.23 | −2.44 | −1.43 | −0.85 |
| V89 | −2.86 | −3.20 | −2.67 | −1.11 |
| V90 | −0.91 | −2.05 | 0.34 | −0.19 |
| V91 | −8.41 | −3.24 | −3.82 | −1.90 |
| V92 | −8.41 | −0.91 | −3.71 | −1.22 |
| V93 | −0.33 | −1.48 | −0.47 | −0.47 |
| V94 | −2.76 | −2.91 | −3.21 | −1.34 |
| V95 | −1.76 | −2.33 | −6.73 | −0.98 |
| V96 | −3.59 | −4.32 | −2.72 | −1.51 |
| V97 | −2.62 | −2.77 | −0.30 | −1.15 |
| V98 | −2.78 | −4.93 | −0.21 | −1.25 |
| V99 | −3.72 | −2.70 | −3.85 | −1.35 |
| V100 | 0.22 | −0.80 | 0.66 | −0.40 |
| V101 | −1.42 | −3.89 | 6.73 | −1.33 |
| V102 | −2.31 | −1.24 | −3.03 | −0.78 |
| V103 | −2.84 | −4.57 | −6.73 | −0.81 |
| V104 | −8.41 | −4.13 | −6.73 | −1.90 |
| V105 | −3.54 | −3.27 | −4.26 | −1.41 |
| V106 | 1.38 | −0.81 | 1.33 | 0.45 |
| V107 | −2.54 | −0.88 | −0.52 | −0.65 |
| V108 | 0.36 | −0.40 | 0.32 | 0.51 |
| V109 | −2.90 | −0.72 | −0.30 | −0.18 |
| V110 | 0.35 | −0.72 | −0.87 | −1.45 |
| V111 | −4.22 | −1.20 | −6.73 | −1.00 |
| V112 | 0.08 | −1.48 | 0.09 | −0.41 |
| V113 | 1.02 | −1.60 | −0.59 | 0.05 |
| V114 | −4.70 | −3.26 | −2.25 | −2.01 |
| V115 | −2.43 | −1.90 | −1.08 | −1.19 |
| V116 | −8.41 | −1.62 | −0.87 | −1.05 |
| V117 | −3.33 | −2.06 | −6.73 | −1.15 |
| V118 | −8.41 | −2.74 | −0.14 | −1.44 |
| V119 | −2.51 | −1.33 | −0.83 | −0.52 |
| V120 | −2.19 | −1.80 | −0.56 | −0.56 |
| V121 | −2.12 | −0.81 | −0.45 | 0.03 |
| V122 | −2.75 | −3.16 | −3.46 | −1.27 |
| V123 | −8.41 | −1.67 | −6.73 | −1.37 |
| V124 | 1.17 | −1.46 | −0.07 | 0.25 |
| V125 | −4.57 | −3.40 | −1.38 | −1.28 |
| V126 | −5.37 | −2.20 | −3.50 | −1.81 |
| V127 | −3.54 | −2.47 | −1.67 | −0.71 |
| V128 | −2.45 | −4.18 | −1.58 | −1.25 |
| V129 | −0.73 | −0.61 | −0.37 | −0.23 |
| V130 | −8.41 | −2.56 | −6.73 | −1.20 |
| V131 | −4.15 | −1.97 | −6.73 | −1.61 |
| V132 | −2.21 | −2.91 | −1.69 | −0.45 |
| V133 | −0.49 | −0.64 | −1.21 | −0.41 |
| V134 | −1.20 | −2.01 | −2.58 | −1.17 |
| V135 | −3.57 | −2.46 | −2.71 | −1.89 |
| V136 | −0.58 | −0.72 | −2.03 | −0.11 |
| V137 | −8.41 | −2.93 | −1.50 | −1.25 |
| V138 | −4.37 | −2.94 | −3.51 | −1.79 |
| V139 | −2.83 | −0.22 | −1.96 | −0.88 |
| V140 | −0.22 | −7.67 | −6.73 | −1.10 |
| V141 | −4.09 | −2.23 | −1.41 | −0.54 |
| V142 | −4.06 | −3.21 | −6.73 | −2.65 |
| V143 | −2.66 | −2.81 | −3.38 | −1.60 |
| V144 | −8.41 | −3.77 | −0.75 | −0.93 |
| V145 | −4.11 | −3.25 | −3.24 | −1.32 |
| V146 | −3.61 | −2.17 | −0.42 | −1.77 |
| V147 | −3.37 | −1.93 | −4.09 | −0.85 |
| V148 | −4.20 | −7.67 | −6.73 | −1.78 |
| V149 | −3.24 | −2.58 | −2.05 | −1.36 |
| V150 | −8.41 | −3.99 | −6.73 | −1.42 |
| Known RGD- | −2.36 | −1.32 | −0.44 | −1.61 |
| insertion | | | | |
| containing capsid | | | | |
| variant | | | | |

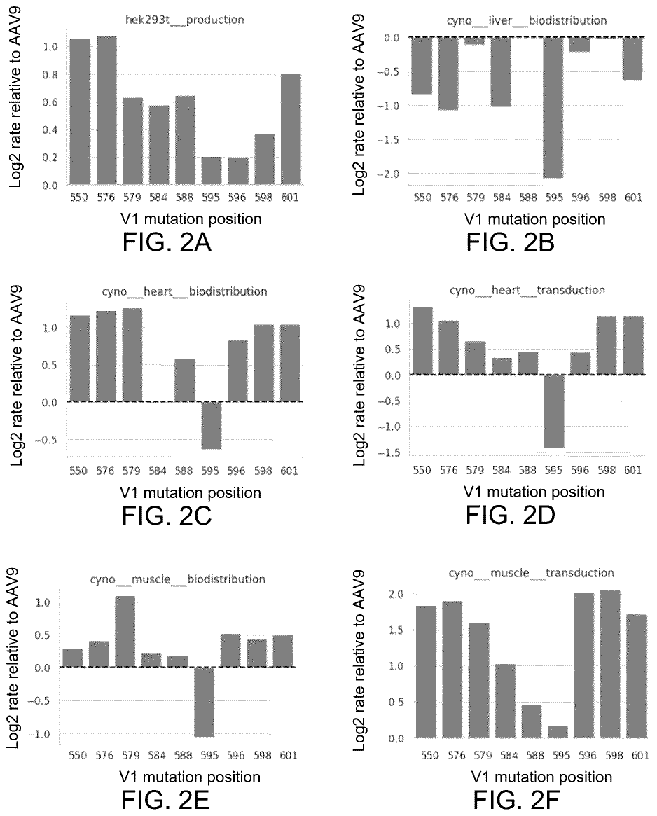

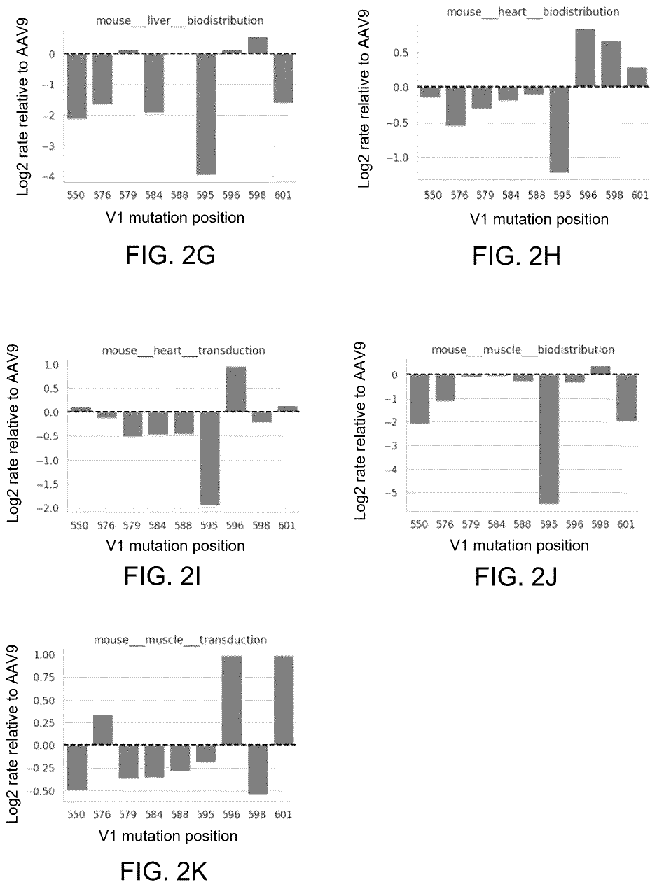

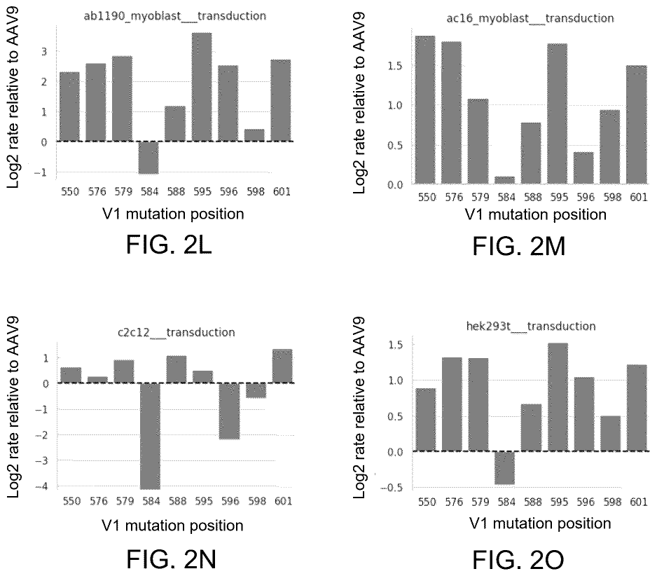

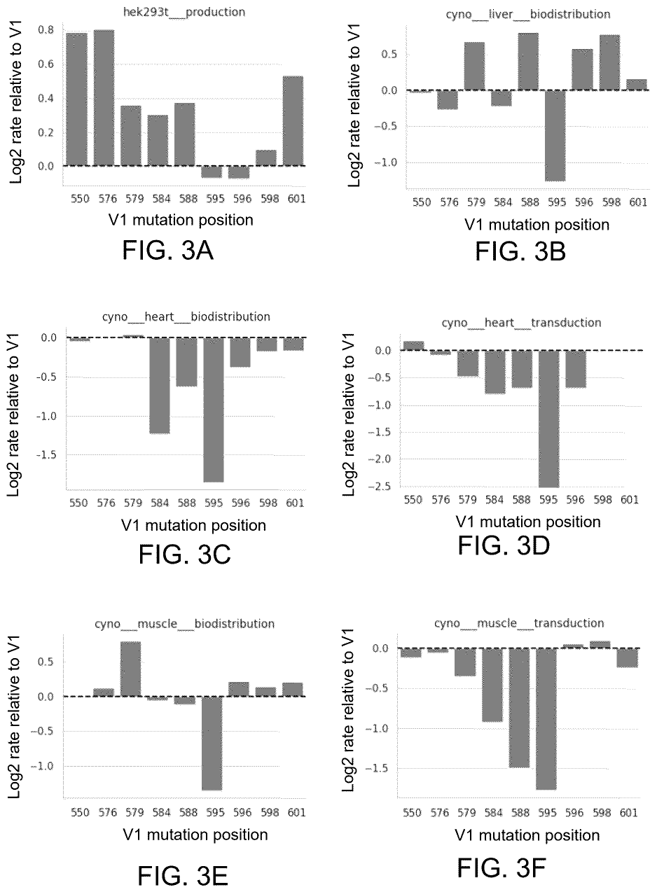

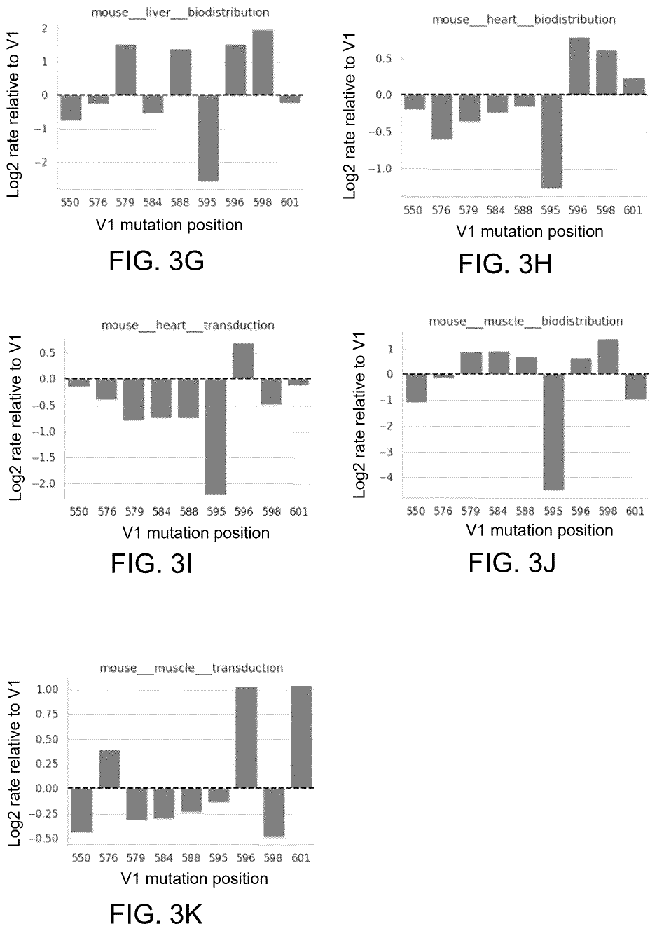

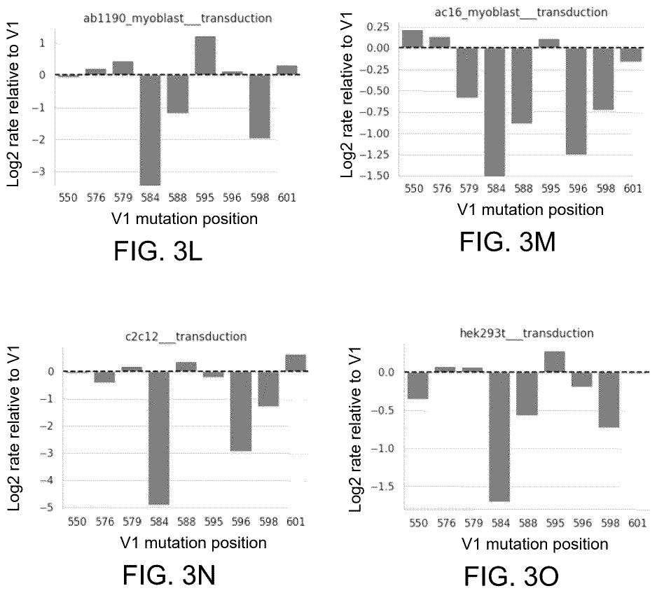

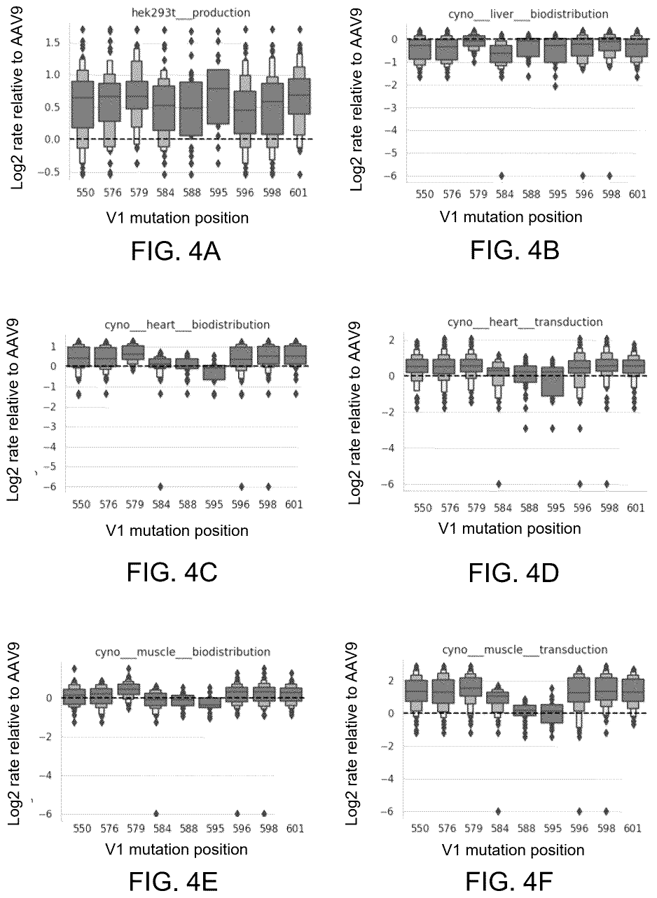

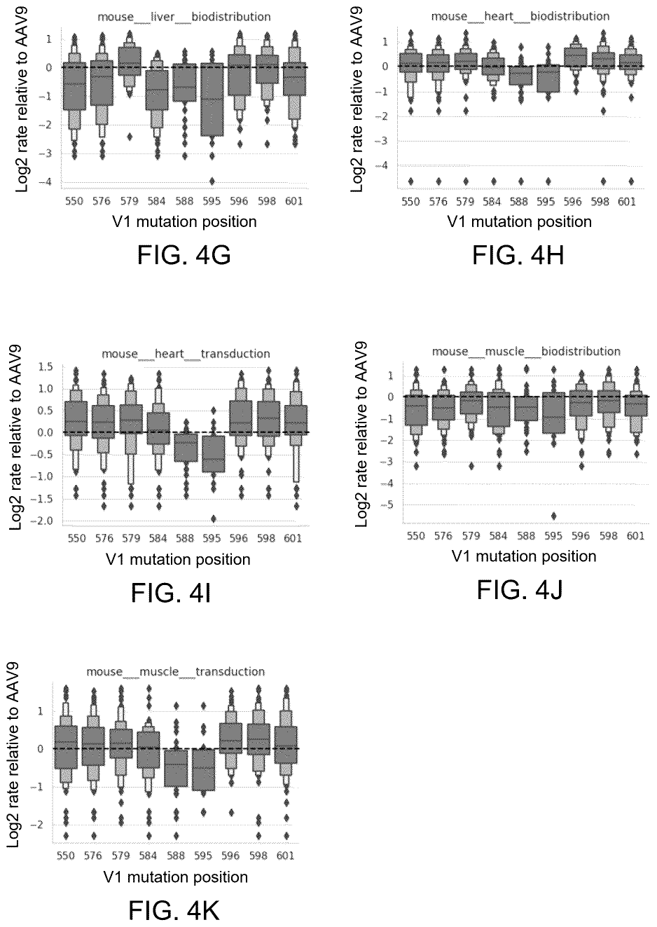

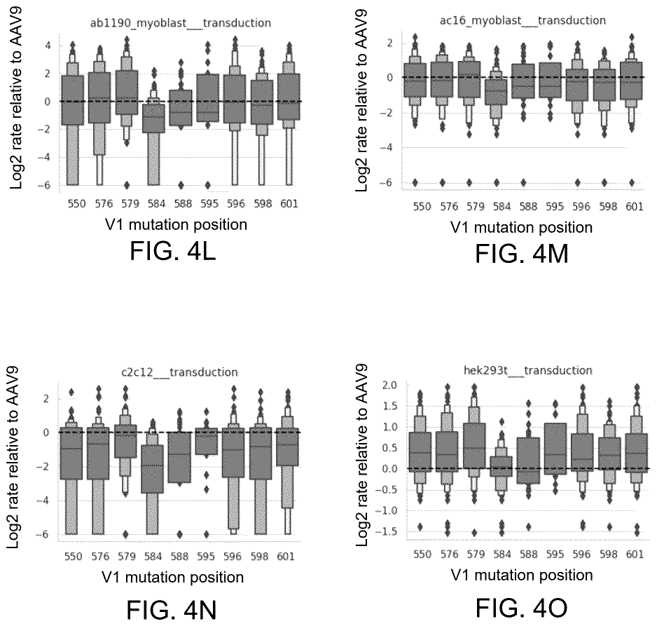

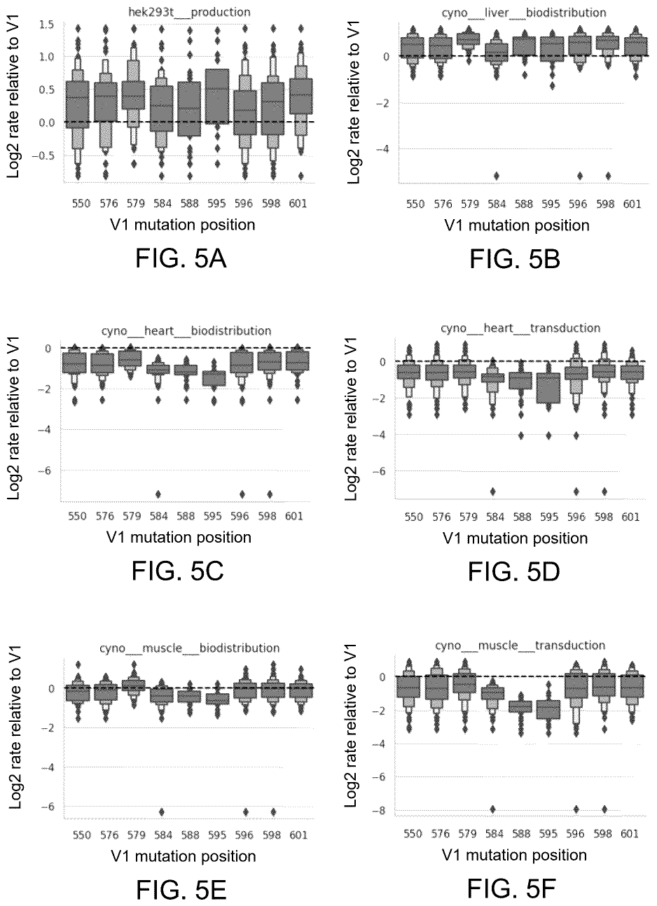

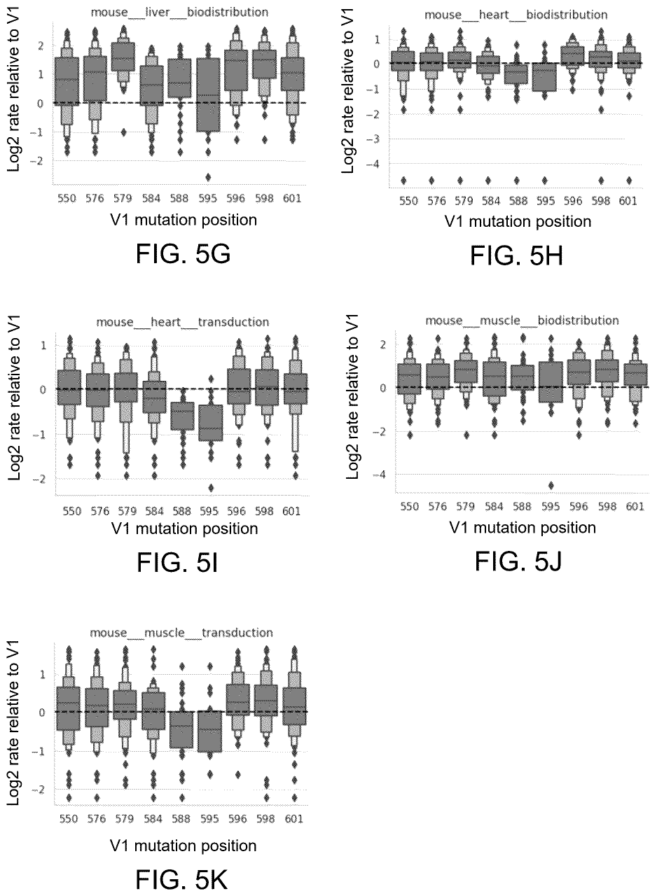

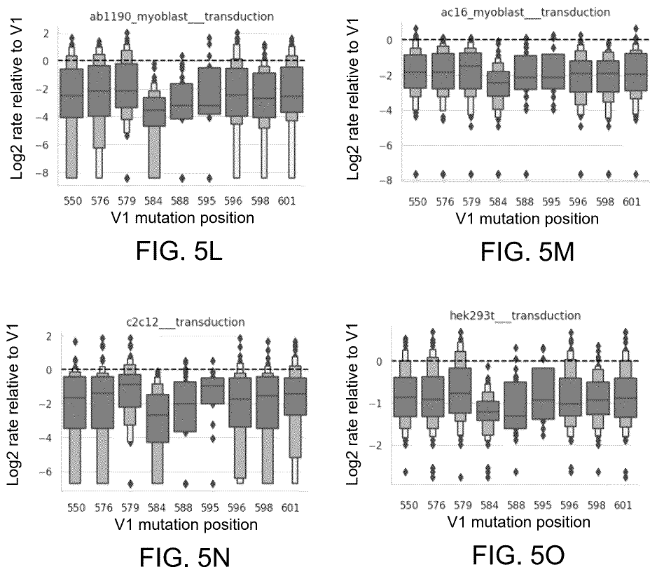

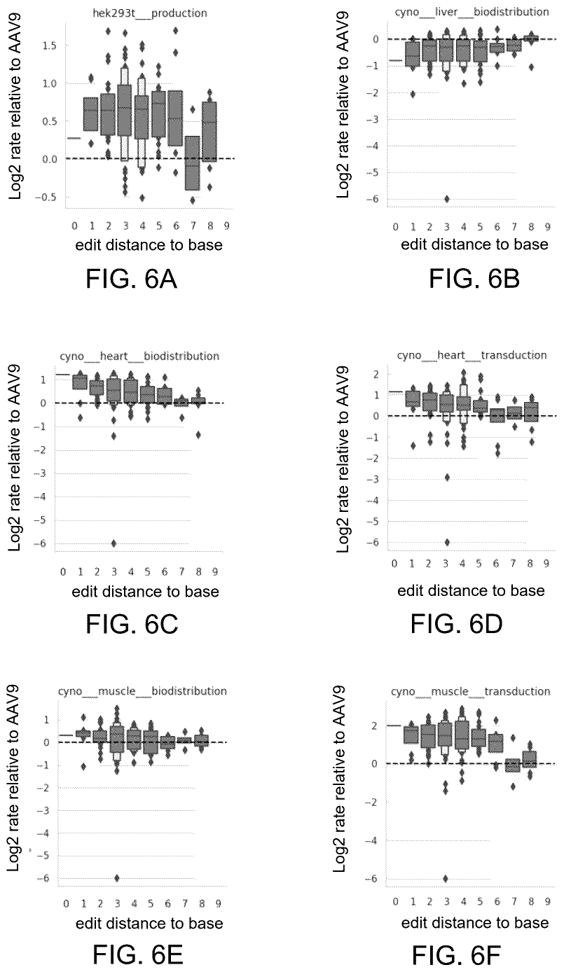

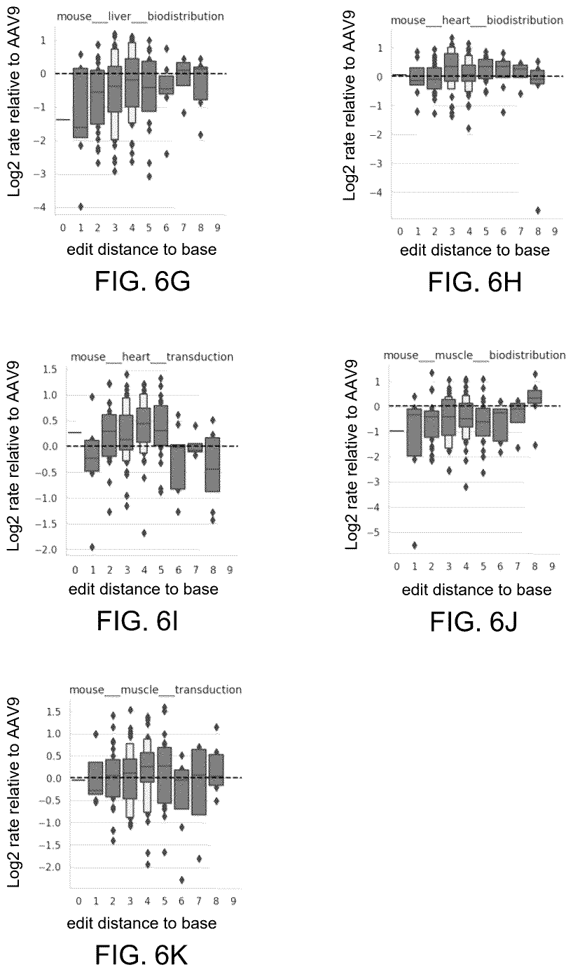

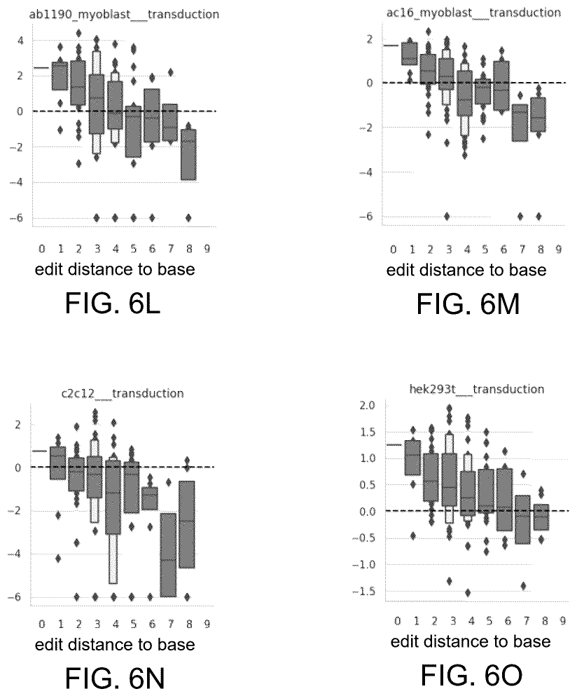

The results show that many variants containing one or more of mutations of V1 target liver less than WT AAV9, target heart (e.g., primate heart or murine heart) more than WT AAV9, target muscle (e.g., primate muscle or murine muscle) more than WT AAV9, and/or target spleen (e.g., primate spleen or murine spleen) less than WT AAV9 with respect to biodistribution and/or transduction. Some evaluated mutations, e.g., H584K, Q588Y, and/or W595A are important for in vivo muscle targeting. The results further show that some evaluated mutations, e.g., Q579T, are important for liver detargeting. The results further show that some evaluated mutations, e.g., R550H, S576A, and/or 1601V, are important for capsid productivity. The results further suggest that some evaluated mutations, e.g., V596L and/or N598S, are important for in vitro muscle targeting of relevant human muscle cell lines (FIGS. 2A-2O, 3A-3O, 4A-4O, and 5A-5O). Furthermore, for some evaluated properties, variant capsids with up to an edit distance of 2, 3, 4, 5, 6, 7, or 8 amino acids showed comparable or improved function relative to WT AAV9 (FIGS. 6A-6O).

### 9. SEQUENCE LISTING

Exemplary sequences of the present disclosure are provided in Table S below, where SEQ=SEQ ID NO.

TABLE S

| Description | Sequence | SEQ |
|---|---|---|
| AAV2 VP1 wild- | MAADGYLPDWLEDTLSEGIRQWWKLKPGPPPPKPAERHKDDSRGLVLPG | 1 |
| type reference | YKYLGPFNGLDKGEPVNEADAAALEHDKAYDRQLDSGDNPYLKYNHADA | |
| sequence | EFQERLKEDTSFGGNLGRAVFQAKKRVLEPLGLVEEPVKTAPGKKRPVEH | |
| (amino acid) | SPVEPDSSSGTGKAGQQPARKRLNFGQTGDADSVPDPQPLGQPPAAPSG | |
| | LGTNTMATGSGAPMADNNEGADGVGNSSGNWHCDSTWMGDRVITTSTR | |
| | TWALPTYNNHLYKQISSQSGASNDNHYFGYSTPWGYFDFNRFHCHFSPR | |
| | DWQRLINNNWGFRPKRLNFKLFNIQVKEVTQNDGTTTIANNLTSTVQVFTD | |
| | SEYQLPYVLGSAHQGCLPPFPADVFMVPQYGYLTLNNGSQAVGRSSFYCL | |
| | EYFPSQMLRTGNNFTFSYTFEDVPFHSSYAHSQSLDRLMNPLIDQYLYYLS | |
| | RTNTPSGTTTQSRLQFSQAGASDIRDQSRNWLPGPCYRQQRVSKTSADN | |
| | NNSEYSWTGATKYHLNGRDSLVNPGPAMASHKDDEEKFFPQSGVLIFGK | |
| | QGSEKTNVDIEKVMITDEEEIRTTNPVATEQYGSVSTNLQRGNRQAATADV | |
| | NTQGVLPGMVWQDRDVYLQGPIWAKIPHTDGHFHPSPLMGGFGLKHPPP | |
| | QILIKNTPVPANPSTTFSAAKFASFITQYSTGQVSVEIEWELQKENSKRWNP | |
| | EIQYTSNYNKSVNVDFTVDTNGVYSEPRPIGTRYLTRNL | |
| AAV2 VP1 wild- | ATGGCTGCCGATGGTTATCTTCCAGATTGGCTCGAGGACACTCTCTCTG | 2 |
| type reference | AAGGAATAAGACAGTGGTGGAAGCTCAAACCTGGCCCACCACCACCAA | |
| sequence | AGCCCGCAGAGCGGCATAAGGACGACAGCAGGGGTCTTGTGCTTCCT | |
| (nucleotide) | GGGTACAAGTACCTCGGACCCTTCAACGGACTCGACAAGGGAGAGCC | |
| | GGTCAACGAGGCAGACGCCGCGGCCCTCGAGCACGACAAAGCCTACG | |
| | ACCGGCAGCTCGACAGCGGAGACAACCCGTACCTCAAGTACAACCACG | |
| | CCGACGCGGAGTTTCAGGAGCGCCTTAAAGAAGATACGTCTTTTGGGG | |
| | GCAACCTCGGACGAGCAGTCTTCCAGGCGAAAAAGAGGGTTCTTGAAC | |
| | CTCTGGGCCTGGTTGAGGAACCTGTTAAGACGGCTCCGGGAAAAAAGA | |
| | GGCCGGTAGAGCACTCTCCTGTGGAGCCAGACTCCTCCTCGGGAACC | |
| | GGAAAGGCGGGCCAGCAGCCTGCAAGAAAAAGATTGAATTTTGGTCAG | |
| | ACTGGAGACGCAGACTCAGTACCTGACCCCCAGCCTCTCGGACAGCCA | |
| | CCAGCAGCCCCCTCTGGTCTGGGAACTAATACGATGGCTACAGGCAGT | |
| | GGCGCACCAATGGCAGACAATAACGAGGGCGCCGACGGAGTGGGTAA | |
| | TTCCTCGGGAAATTGGCATTGCGATTCCACATGGATGGGCGACAGAGT | |
| | CATCACCACCAGCACCCGAACCTGGGCCCTGCCCACCTACAACAACCA | |
| | CCTCTACAAACAAATTTCCAGCCAATCAGGAGCCTCGAACGACAATCAC | |
| | TACTTTGGCTACAGCACCCCTTGGGGGTATTTTGACTTCAACAGATTCC | |
| | ACTGCCACTTTTCACCACGTGACTGGCAAAGACTCATCAACAACAACTG | |
| | GGGATTCCGACCCAAGAGACTCAACTTCAAGCTCTTTAACATTCAAGTC | |
| | AAAGAGGTCACGCAGAATGACGGTACGACGACGATTGCCAATAACCTT | |
| | ACCAGCACGGTTCAGGTGTTTACTGACTCGGAGTACCAGCTCCCGTAC | |
| | GTCCTCGGCTCGGCGCATCAAGGATGCCTCCCGCCGTTCCCAGCAGA | |
| | CGTCTTCATGGTGCCACAGTATGGATACCTCACCCTGAACAACGGGAG | |
| | TCAGGCAGTAGGACGCTCTTCATTTTACTGCCTGGAGTACTTTCCTTCT | |
| | CAGATGCTGCGTACCGGAAACAACTTTACCTTCAGCTACACTTTTGAGG | |
| | ACGTTCCTTTCCACAGCAGCTACGCTCACAGCCAGAGTCTGGACCGTC | |
| | TCATGAATCCTCTCATCGACCAGTACCTGTATTACTTGAGCAGAACAAA | |
| | CACTCCAAGTGGAACCACCACGCAGTCAAGGCTTCAGTTTTCTCAGGC | |
| | CGGAGCGAGTGACATTCGGGACCAGTCTAGGAACTGGCTTCCTGGACC | |
| | CTGTTACCGCCAGCAGCGAGTATCAAAGACATCTGCGGATAACAACAA | |
| | CAGTGAATACTCGTGGACTGGAGCTACCAAGTACCACCTCAATGGCAG | |
| | AGACTCTCTGGTGAATCCGGGCCCGGCCATGGCAAGCCACAAGGACG | |
| | ATGAAGAAAAGTTTTTTCCTCAGAGCGGGGTTCTCATCTTTGGGAAGCA | |
| | AGGCTCAGAGAAAACAAATGTGGACATTGAAAAGGTCATGATTACAGAC | |
| | GAAGAGGAAATCAGGACAACCAATCCCGTGGCTACGGAGCAGTATGGT | |
| | TCTGTATCTACCAACCTCCAGAGAGGCAACAGACAAGCAGCTACCGCA | |
| | GATGTCAACACACAAGGCGTTCTTCCAGGCATGGTCTGGCAGGACAGA | |
| | GATGTGTACCTTCAGGGGCCCATCTGGGCAAAGATTCCACACACGGAC | |
| | GGACATTTTCACCCCTCTCCCCTCATGGGTGGATTCGGACTTAAACACC | |
| | CTCCTCCACAGATTCTCATCAAGAACACCCCGGTACCTGCGAATCCTTC | |
| | GACCACCTTCAGTGCGGCAAAGTTTGCTTCCTTCATCACACAGTACTCC | |
| | ACGGGACAGGTCAGCGTGGAGATCGAGTGGGAGCTGCAGAAGGAAAA | |
| | CAGCAAACGCTGGAATCCCGAAATTCAGTACACTTCCAACTACAACAAG | |
| | TCTGTTAATGTGGACTTTACTGTGGACACTAATGGCGTGTATTCAGAGC | |
| | CTCGCCCCATTGGCACCAGATACCTGACTCGTAATCTGTAA | |
| AAV5 VP1 wild- | MSFVDHPPDWLEEVGEGLREFLGLEAGPPKPKPNQQHQDQARGLVLPGY | 3 |
| type reference | NYLGPGNGLDRGEPVNRADEVAREHDISYNEQLEAGDNPYLKYNHADAEF | |
| sequence | QEKLADDTSFGGNLGKAVFQAKKRVLEPFGLVEEGAKTAPTGKRIDDHFP | |
| (amino acid) | KRKKARTEEDSKPSTSSDAEAGPSGSQQLQIPAQPASSLGADTMSAGGG | |
| | GPLGDNNQGADGVGNASGDWHCDSTWMGDRVVTKSTRTWVLPSYNNH | |
| | QYREIKSGSVDGSNANAYFGYSTPWGYFDFNRFHSHWSPRDWQRLINNY | |
| | WGFRPRSLRVKIFNIQVKEVTVQDSTTTIANNLTSTVQVFTDDDYQLPYVV | |
| | GNGTEGCLPAFPPQVFTLPQYGYATLNRDNTENPTERSSFFCLEYFPSKM | |
| | LRTGNNFEFTYNFEEVPFHSSFAPSQNLFKLANPLVDQYLYRFVSTNNTGG | |
| | VQFNKNLAGRYANTYKNWFPGPMGRTQGWNLGSGVNRASVSAFATTNR | |
| | MELEGASYQVPPQPNGMTNNLQGSNTYALENTMIFNSQPANPGTTATYLE | |
| | GNMLITSESETQPVNRVAYNVGGQMATNNQSSTTAPATGTYNLQEIVPGS | |
| | VWMERDVYLQGPIWAKIPETGAHFHPSPAMGGFGLKHPPPMMLIKNTPVP | |
| | GNITSFSDVPVSSFITQYSTGQVTVEMEWELKKENSKRWNPEIQYTNNYN | |
| | DPQFVDFAPDSTGEYRTTRPIGTRYLTRPL | |
| AAV5 VP1 wild- | ATGTCTTTTGTTGATCACCCTCCAGATTGGTTGGAAGAAGTTGGTGAAG | 4 |
| type reference | GTCTTCGCGAGTTTTTGGGCCTTGAAGCGGGCCCACCGAAACCAAAAC | |
| sequence | CCAATCAGCAGCATCAAGATCAAGCCCGTGGTCTTGTGCTGCCTGGTT | |
| (nucleotide) | ATAACTATCTCGGACCCGGAAACGGGCTCGATCGAGGAGAGCCTGTCA | |
| | ACAGGGCAGACGAGGTCGCGCGAGAGCACGACATCTCGTACAACGAG | |
| | CAGCTTGAGGCGGGAGACAACCCCTACCTCAAGTACAACCACGCGGAC | |
| | GCCGAGTTTCAGGAGAAGCTCGCCGACGACACATCCTTCGGGGGAAA | |
| | CCTCGGAAAGGCAGTCTTTCAGGCCAAGAAAAGGGTTCTCGAACCTTTT | |
| | GGCCTGGTTGAAGAGGGTGCTAAGACGGCCCCTACCGGAAAGCGGAT | |
| | AGACGACCACTTTCCAAAAAGAAAGAAGGCTCGGACCGAAGAGGACTC | |
| | CAAGCCTTCCACCTCGTCAGACGCCGAAGCTGGACCCAGCGGATCCCA | |
| | GCAGCTGCAAATCCCAGCCCAACCAGCCTCAAGTTTGGGAGCTGATAC | |
| | AATGTCTGCGGGAGGTGGCGGCCCATTGGGCGACAATAACCAAGGTG | |
| | CCGATGGAGTGGGCAATGCCTCGGGAGATTGGCATTGCGATTCCACGT | |
| | GGATGGGGGACAGAGTCGTCACCAAGTCCACCCGAACCTGGGTGCTG | |
| | CCCAGCTACAACAACCACCAGTACCGAGAGATCAAAAGCGGCTCCGTC | |
| | GACGGAAGCAACGCCAACGCCTACTTTGGATACAGCACCCCCTGGGG | |
| | GTACTTTGACTTTAACCGCTTCCACAGCCACTGGAGCCCCCGAGACTG | |
| | GCAAAGACTCATCAACAACTACTGGGGCTTCAGACCCCGGTCCCTCAG | |
| | AGTCAAAATCTTCAACATTCAAGTCAAAGAGGTCACGGTGCAGGACTCC | |
| | ACCACCACCATCGCCAACAACCTCACCTCCACCGTCCAAGTGTTTACG | |
| | GACGACGACTACCAGCTGCCCTACGTCGTCGGCAACGGGACCGAGGG | |
| | ATGCCTGCCGGCCTTCCCTCCGCAGGTCTTTACGCTGCCGCAGTACGG | |
| | TTACGCGACGCTGAACCGCGACAACACAGAAAATCCCACCGAGAGGAG | |
| | CAGCTTCTTCTGCCTAGAGTACTTTCCCAGCAAGATGCTGAGAACGGG | |
| | CAACAACTTTGAGTTTACCTACAACTTTGAGGAGGTGCCCTTCCACTCC | |
| | AGCTTCGCTCCCAGTCAGAACCTGTTCAAGCTGGCCAACCCGCTGGTG | |
| | GACCAGTACTTGTACCGCTTCGTGAGCACAAATAACACTGGCGGAGTC | |
| | CAGTTCAACAAGAACCTGGCCGGGAGATACGCCAACACCTACAAAAAC | |
| | TGGTTCCCGGGGCCCATGGGCCGAACCCAGGGCTGGAACCTGGGCTC | |
| | CGGGGTCAACCGCGCCAGTGTCAGCGCCTTCGCCACGACCAATAGGA | |
| | TGGAGCTCGAGGGCGCGAGTTACCAGGTGCCCCCGCAGCCGAACGGC | |
| | ATGACCAACAACCTCCAGGGCAGCAACACCTATGCCCTGGAGAACACT | |
| | ATGATCTTCAACAGCCAGCCGGCGAACCCGGGCACCACCGCCACGTA | |
| | CCTCGAGGGCAACATGCTCATCACCAGCGAGAGCGAGACGCAGCCGG | |
| | TGAACCGCGTGGCGTACAACGTCGGCGGGCAGATGGCCACCAACAAC | |
| | CAGAGCTCCACCACTGCCCCCGCGACCGGCACGTACAACCTCCAGGA | |
| | AATCGTGCCCGGCAGCGTGTGGATGGAGAGGGACGTGTACCTCCAAG | |
| | GACCCATCTGGGCCAAGATCCCAGAGACGGGGGCGCACTTTCACCCC | |
| | TCTCCGGCCATGGGGGGATTCGGACTCAAACACCCACCGCCCATGATG | |
| | CTCATCAAGAACACGCCTGTGCCCGGAAATATCACCAGCTTCTCGGAC | |
| | GTGCCCGTCAGCAGCTTCATCACCCAGTACAGCACCGGGCAGGTCACC | |
| | GTGGAGATGGAGTGGGAGCTCAAGAAGGAAAACTCCAAGAGGTGGAA | |
| | CCCAGAGATCCAGTACACAAACAACTACAACGACCCCCAGTTTGTGGA | |
| | CTTTGCCCCGGACAGCACCGGGGAATACAGAACCACCAGACCTATCGG | |
| | AACCCGATACCTTACCCGACCCCTTTAA | |
| AAV8 VP1 wild- | MAADGYLPDWLEDNLSEGIREWWALKPGAPKPKANQQKQDDGRGLVLP | 5 |
| type reference | GYKYLGPFNGLDKGEPVNAADAAALEHDKAYDQQLQAGDNPYLRYNHAD | |
| sequence | AEFQERLQEDTSFGGNLGRAVFQAKKRVLEPLGLVEEGAKTAPGKKRPVE | |
| (amino acid) | PSPQRSPDSSTGIGKKGQQPARKRLNFGQTGDSESVPDPQPLGEPPAAP | |
| | SGVGPNTMAAGGGAPMADNNEGADGVGSSSGNWHCDSTWLGDRVITTS | |
| | TRTWALPTYNNHLYKQISNGTSGGATNDNTYFGYSTPWGYFDFNRFHCH | |
| | FSPRDWQRLINNNWGFRPKRLSFKLFNIQVKEVTQNEGTKTIANNLTSTIQ | |
| | VFTDSEYQLPYVLGSAHQGCLPPFPADVFMIPQYGYLTLNNGSQAVGRSS | |
| | FYCLEYFPSQMLRTGNNFQFTYTFEDVPFHSSYAHSQSLDRLMNPLIDQYL | |
| | YYLSRTQTTGGTANTQTLGFSQGGPNTMANQAKNWLPGPCYRQQRVSTT | |
| | TGQNNNSNFAWTAGTKYHLNGRNSLANPGIAMATHKDDEERFFPSNGILIF | |
| | GKQNAARDNADYSDVMLTSEEEIKTTNPVATEEYGIVADNLQQQNTAPQIG | |
| | TVNSQGALPGMVWQNRDVYLQGPIWAKIPHTDGNFHPSPLMGGFGLKHP | |
| | PPQILIKNTPVPADPPTTFNQSKLNSFITQYSTGQVSVEIEWELQKENSKRW | |
| | NPEIQYTSNYYKSTSVDFAVNTEGVYSEPRPIGTRYLTRNL | |
| AAV8 VP1 wild- | ATGGCTGCCGATGGTTATCTTCCAGATTGGCTCGAGGACAACCTCTCT | 6 |
| type reference | GAGGGCATTCGCGAGTGGTGGGCGCTGAAACCTGGAGCCCCGAAGCC | |
| sequence | CAAAGCCAACCAGCAAAAGCAGGACGACGGCCGGGGTCTGGTGCTTC | |
| (nucleotide) | CTGGCTACAAGTACCTCGGACCCTTCAACGGACTCGACAAGGGGGAGC | |
| | CCGTCAACGCGGCGGACGCAGCGGCCCTCGAGCACGACAAGGCCTAC | |
| | GACCAGCAGCTGCAGGCGGGTGACAATCCGTACCTGCGGTATAACCAC | |
| | GCCGACGCCGAGTTTCAGGAGCGTCTGCAAGAAGATACGTCTTTTGGG | |
| | GGCAACCTCGGGCGAGCAGTCTTCCAGGCCAAGAAGCGGGTTCTCGA | |
| | ACCTCTCGGTCTGGTTGAGGAAGGCGCTAAGACGGCTCCTGGAAAGAA | |
| | GAGACCGGTAGAGCCATCACCCCAGCGTTCTCCAGACTCCTCTACGGG | |
| | CATCGGCAAGAAAGGCCAACAGCCCGCCAGAAAAAGACTCAATTTTGG | |
| | TCAGACTGGCGACTCAGAGTCAGTTCCAGACCCTCAACCTCTCGGAGA | |
| | ACCTCCAGCAGCGCCCTCTGGTGTGGGACCTAATACAATGGCTGCAGG | |
| | CGGTGGCGCACCAATGGCAGACAATAACGAAGGCGCCGACGGAGTGG | |
| | GTAGTTCCTCGGGAAATTGGCATTGCGATTCCACATGGCTGGGCGACA | |
| | GAGTCATCACCACCAGCACCCGAACCTGGGCCCTGCCCACCTACAACA | |
| | ACCACCTCTACAAGCAAATCTCCAACGGGACATCGGGAGGAGCCACCA | |
| | ACGACAACACCTACTTCGGCTACAGCACCCCCTGGGGGTATTTTGACTT | |
| | TAACAGATTCCACTGCCACTTTTCACCACGTGACTGGCAGCGACTCATC | |
| | AACAACAACTGGGGATTCCGGCCCAAGAGACTCAGCTTCAAGCTCTTC | |
| | AACATCCAGGTCAAGGAGGTCACGCAGAATGAAGGCACCAAGACCATC | |
| | GCCAATAACCTCACCAGCACCATCCAGGTGTTTACGGACTCGGAGTAC | |
| | CAGCTGCCGTACGTTCTCGGCTCTGCCCACCAGGGCTGCCTGCCTCC | |
| | GTTCCCGGCGGACGTGTTCATGATTCCCCAGTACGGCTACCTAACACT | |
| | CAACAACGGTAGTCAGGCCGTGGGACGCTCCTCCTTCTACTGCCTGGA | |
| | ATACTTTCCTTCGCAGATGCTGAGAACCGGCAACAACTTCCAGTTTACT | |
| | TACACCTTCGAGGACGTGCCTTTCCACAGCAGCTACGCCCACAGCCAG | |
| | AGCTTGGACCGGCTGATGAATCCTCTGATTGACCAGTACCTGTACTACT | |
| | TGTCTCGGACTCAAACAACAGGAGGCACGGCAAATACGCAGACTCTGG | |
| | GCTTCAGCCAAGGTGGGCCTAATACAATGGCCAATCAGGCAAAGAACT | |
| | GGCTGCCAGGACCCTGTTACCGCCAACAACGCGTCTCAACGACAACCG | |
| | GGCAAAACAACAATAGCAACTTTGCCTGGACTGCTGGGACCAAATACC | |
| | ATCTGAATGGAAGAAATTCATTGGCTAATCCTGGCATCGCTATGGCAAC | |
| | ACACAAAGACGACGAGGAGCGTTTTTTTCCCAGTAACGGGATCCTGATT | |
| | TTTGGCAAACAAAATGCTGCCAGAGACAATGCGGATTACAGCGATGTCA | |
| | TGCTCACCAGCGAGGAAGAAATCAAAACCACTAACCCTGTGGCTACAG | |
| | AGGAATACGGTATCGTGGCAGATAACTTGCAGCAGCAAAACACGGCTC | |
| | CTCAAATTGGAACTGTCAACAGCCAGGGGGCCTTACCCGGTATGGTCT | |
| | GGCAGAACCGGGACGTGTACCTGCAGGGTCCCATCTGGGCCAAGATT | |
| | CCTCACACGGACGGCAACTTCCACCCGTCTCCGCTGATGGGGGGCTTT | |
| | GGCCTGAAACATCCTCCGCCTCAGATCCTGATCAAGAACACGCCTGTA | |
| | CCTGCGGATCCTCCGACCACCTTCAACCAGTCAAAGCTGAACTCTTTCA | |
| | TCACGCAATACAGCACCGGACAGGTCAGCGTGGAAATTGAATGGGAGC | |
| | TGCAGAAGGAAAACAGCAAGCGCTGGAACCCCGAGATCCAGTACACCT | |
| | CCAACTACTACAAATCTACAAGTGTGGACTTTGCTGTTAATACAGAAGG | |
| | CGTGTACTCTGAACCCCGCCCCATTGGCACCCGTTACCTCACCCGTAA | |
| | TCTGTAA | |
| AAV9 VP1 wild- | MAADGYLPDWLEDNLSEGIREWWALKPGAPQPKANQQHQDNARGLVLP | 7 |
| type reference | GYKYLGPGNGLDKGEPVNAADAAALEHDKAYDQQLKAGDNPYLKYNHAD | |
| sequence | AEFQERLKEDTSFGGNLGRAVFQAKKRLLEPLGLVEEAAKTAPGKKRPVE | |
| (amino acid) | QSPQEPDSSAGIGKSGAQPAKKRLNFGQTGDTESVPDPQPIGEPPAAPSG | |
| | VGSLTMASGGGAPVADNNEGADGVGSSSGNWHCDSQWLGDRVITTSTR | |
| | TWALPTYNNHLYKQISNSTSGGSSNDNAYFGYSTPWGYFDFNRFHCHFS | |
| | PRDWQRLINNNWGFRPKRLNFKLFNIQVKEVTDNNGVKTIANNLTSTVQVF | |
| | TDSDYQLPYVLGSAHEGCLPPFPADVFMIPQYGYLTLNDGSQAVGRSSFY | |
| | CLEYFPSQMLRTGNNFQFSYEFENVPFHSSYAHSQSLDRLMNPLIDQYLY | |
| | YLSKTINGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGSLIFG | |
| | KQGTGRDNVDADKVMITNEEEIKTTNPVATESYGQVATNHQSAQAQAQTG | |
| | WVQNQGILPGMVWQDRDVYLQGPIWAKIPHTDGNFHPSPLMGGFGMKH | |
| | PPPQILIKNTPVPADPPTAFNKDKLNSFITQYSTGQVSVEIEWELQKENSKR | |
| | WNPEIQYTSNYYKSNNVEFAVNTEGVYSEPRPIGTRYLTRNL | |
| AAV9 VP1 wild- | ATGGCTGCCGATGGTTATCTTCCAGATTGGCTCGAGGACAACCTTAGT | 8 |
| type reference | GAAGGTATTCGCGAGTGGTGGGCTTTGAAACCTGGAGCCCCTCAACCC | |
| sequence | AAGGCAAATCAACAACATCAAGACAACGCTCGAGGTCTTGTGCTTCCG | |
| (nucleotide) | GGTTACAAATACCTTGGACCCGGCAACGGACTCGACAAGGGGGAGCC | |
| | GGTCAACGCAGCAGACGCGGCGGCCCTCGAGCACGACAAGGCCTACG | |
| | ACCAGCAGCTCAAGGCCGGAGACAACCCGTACCTCAAGTACAACCACG | |
| | CCGACGCCGAGTTCCAGGAGCGGCTCAAAGAAGATACGTCTTTTGGGG | |
| | GCAACCTCGGGCGAGCAGTCTTCCAGGCCAAAAAGAGGCTTCTTGAAC | |
| | CTCTTGGTCTGGTTGAGGAAGCGGCTAAGACGGCTCCTGGAAAGAAGA | |
| | GGCCTGTAGAGCAGTCTCCTCAGGAACCGGACTCCTCCGCGGGTATTG | |
| | GCAAATCGGGTGCACAGCCCGCTAAAAAGAGACTCAATTTCGGTCAGA | |
| | CTGGCGACACAGAGTCAGTCCCAGACCCTCAACCAATCGGAGAACCTC | |
| | CCGCAGCCCCCTCAGGTGTGGGATCTCTTACAATGGCTTCAGGTGGTG | |
| | GCGCACCAGTGGCAGACAATAACGAAGGTGCCGATGGAGTGGGTAGT | |
| | TCCTCGGGAAATTGGCATTGCGATTCCCAATGGCTGGGGGACAGAGTC | |
| | ATCACCACCAGCACCCGAACCTGGGCCCTGCCCACCTACAACAATCAC | |
| | CTCTACAAGCAAATCTCCAACAGCACATCTGGAGGATCTTCAAATGACA | |
| | ACGCCTACTTCGGCTACAGCACCCCCTGGGGGTATTTTGACTTCAACA | |
| | GATTCCACTGCCACTTCTCACCACGTGACTGGCAGCGACTCATCAACAA | |
| | CAACTGGGGATTCCGGCCTAAGCGACTCAACTTCAAGCTCTTCAACATT | |
| | CAGGTCAAAGAGGTTACGGACAACAATGGAGTCAAGACCATCGCCAAT | |
| | AACCTTACCAGCACGGTCCAGGTCTTCACGGACTCAGACTATCAGCTC | |
| | CCGTACGTGCTCGGGTCGGCTCACGAGGGCTGCCTCCCGCCGTTCCC | |
| | AGCGGACGTTTTCATGATTCCTCAGTACGGGTATCTGACGCTTAATGAT | |
| | GGAAGCCAGGCCGTGGGTCGTTCGTCCTTTTACTGCCTGGAATATTTC | |
| | CCGTCGCAAATGCTAAGAACGGGTAACAACTTCCAGTTCAGCTACGAG | |
| | TTTGAGAACGTACCTTTCCATAGCAGCTACGCTCACAGCCAAAGCCTGG | |
| | ACCGACTAATGAATCCACTCATCGACCAATACTTGTACTATCTCTCAAAG | |
| | ACTATTAACGGTTCTGGACAGAATCAACAAACGCTAAAATTCAGTGTGG | |
| | CCGGACCCAGCAACATGGCTGTCCAGGGAAGAAACTACATACCTGGAC | |
| | CCAGCTACCGACAACAACGTGTCTCAACCACTGTGACTCAAAACAACAA | |
| | CAGCGAATTTGCTTGGCCTGGAGCTTCTTCTTGGGCTCTCAATGGACGT | |
| | AATAGCTTGATGAATCCTGGACCTGCTATGGCCAGCCACAAAGAAGGA | |
| | GAGGACCGTTTCTTTCCTTTGTCTGGATCTTTAATTTTTGGCAAACAAGG | |
| | AACTGGAAGAGACAACGTGGATGCGGACAAAGTCATGATAACCAACGA | |
| | AGAAGAAATTAAAACTACTAACCCGGTAGCAACGGAGTCCTATGGACAA | |
| | GTGGCCACAAACCACCAGAGTGCCCAAGCACAGGCGCAGACCGGCTG | |
| | GGTTCAAAACCAAGGAATACTTCCGGGTATGGTTTGGCAGGACAGAGA | |
| | TGTGTACCTGCAAGGACCCATTTGGGCCAAAATTCCTCACACGGACGG | |
| | CAACTTTCACCCTTCTCCGCTGATGGGAGGGTTTGGAATGAAGCACCC | |
| | GCCTCCTCAGATCCTCATCAAAAACACACCTGTACCTGCGGATCCTCCA | |
| | ACGGCCTTCAACAAGGACAAGCTGAACTCTTTCATCACCCAGTATTCTA | |
| | CTGGCCAAGTCAGCGTGGAGATCGAGTGGGAGCTGCAGAAGGAAAAC | |
| | AGCAAGCGCTGGAACCCGGAGATCCAGTACACTTCCAACTATTACAAG | |
| | TCTAATAATGTTGAATTTGCTGTTAATACTGAAGGTGTATATAGTGAACC | |
| | CCGCCCCATTGGCACCAGATACCTGACTCGTAATCTGTAA | |
| AAVrh74 VP1 | MAADGYLPDWLEDNLSEGIREWWDLKPGAPKPKANQQKQDNGRGLVLP | 9 |
| wild-type | GYKYLGPFNGLDKGEPVNAADAAALEHDKAYDQQLQAGDNPYLRYNHAD | |
| reference | AEFQERLQEDTSFGGNLGRAVFQAKKRVLEPLGLVESPVKTAPGKKRPVE | |
| sequence | PSPQRSPDSSTGIGKKGQQPAKKRLNFGQTGDSESVPDPQPIGEPPAGPS | |
| (amino acid) | GLGSGTMAAGGGAPMADNNEGADGVGSSSGNWHCDSTWLGDRVITTST | |
| | RTWALPTYNNHLYKQISNGTSGGSTNDNTYFGYSTPWGYFDFNRFHCHF | |
| | SPRDWQRLINNNWGFRPKRLNFKLFNIQVKEVTQNEGTKTIANNLTSTIQV | |
| | FTDSEYQLPYVLGSAHQGCLPPFPADVFMIPQYGYLTLNNGSQAVGRSSF | |
| | YCLEYFPSQMLRTGNNFEFSYNFEDVPFHSSYAHSQSLDRLMNPLIDQYL | |
| | YYLSRTQSTGGTAGTQQLLFSQAGPNNMSAQAKNWLPGPCYRQQRVSTT | |
| | LSQNNNSNFAWTGATKYHLNGRDSLVNPGVAMATHKDDEERFFPSSGVL | |
| | MFGKQGAGKDNVDYSSVMLTSEEEIKTTNPVATEQYGVVADNLQQQNAA | |
| | PIVGAVNSQGALPGMVWQNRDVYLQGPIWAKIPHTDGNFHPSPLMGGFG | |
| | LKHPPPQILIKNTPVPADPPTTFNQAKLASFITQYSTGQVSVEIEWELQKEN | |
| | SKRWNPEIQYTSNYYKSTNVDFAVNTEGTYSEPRPIGTRYLTRNL | |
| AAVrh74 VP1 | ATGGCTGCCGATGGTTATCTTCCAGATTGGCTCGAGGACAACCTCTCT | 10 |
| wild-type | GAGGGCATTCGCGAGTGGTGGGACCTGAAACCTGGAGCCCCGAAACC | |
| reference | CAAAGCCAACCAGCAAAAGCAGGACAACGGCCGGGGTCTGGTGCTTC | |
| sequence | CTGGCTACAAGTACCTCGGACCCTTCAACGGACTCGACAAGGGGGAGC | |
| (nucleotide) | CCGTCAACGCGGCGGACGCAGCGGCCCTCGAGCACGACAAGGCCTAC | |
| | GACCAGCAGCTCCAAGCGGGTGACAATCCGTACCTGCGGTATAATCAC | |
| | GCCGACGCCGAGTTTCAGGAGCGTCTGCAAGAAGATACGTCTTTTGGG | |
| | GGCAACCTCGGGCGCGCAGTCTTCCAGGCCAAAAAGCGGGTTCTQGA | |
| | ACCTCTGGGCCTGGTTGAATCGCCGGTTAAGACGGCTCCTGGAAAGAA | |
| | GAGGCCGGTAGAGCCATCACCCCAGCGCTCTCCAGACTCCTCTACGG | |
| | GCATCGGCAAGAAAGGCCAGCAGCCCGCAAAAAAGAGACTCAATTTTG | |
| | GGCAGACTGGCGACTCAGAGTCAGTCCCCGACCCTCAACCAATCGGA | |
| | GAACCACCAGCAGGCCCCTCTGGTCTGGGATCTGGTACAATGGCTGCA | |
| | GGCGGTGGCGCTCCAATGGCAGACAATAACGAAGGCGCCGACGGAGT | |
| | GGGTAGTTCCTCAGGAAATTGGCATTGCGATTCCACATGGCTGGGCGA | |
| | CAGAGTCATCACCACCAGCACCCGCACCTGGGCCCTGCCCACCTACAA | |
| | CAACCACCTCTACAAGCAAATCTCCAACGGGACCTCGGGAGGAAGCAC | |
| | CAACGACAACACCTACTTCGGCTACAGCACCCCCTGGGGGTATTTTGA | |
| | CTTCAACAGATTCCACTGCCACTTTTCACCACGTGACTGGCAGCGACTC | |
| | ATCAACAACAACTGGGGATTCCGGCCCAAGAGGCTCAACTTCAAGCTC | |
| | TTCAACATCCAAGTCAAGGAGGTCACGCAGAATGAAGGCACCAAGACC | |
| | ATCGCCAATAACCTTACCAGCACGATTCAGGTCTTTACGGACTCGGAAT | |
| | ACCAGCTCCCGTACGTGCTCGGCTCGGCGCACCAGGGCTGCCTGCCT | |
| | CCGTTCCCGGCGGACGTCTTCATGATTCCTCAGTACGGGTACCTGACT | |
| | CTGAACAATGGCAGTCAGGCTGTGGGCCGGTCGTCCTTCTACTGCCTG | |
| | GAGTACTTTCCTTCTCAAATGCTGAGAACGGGCAACAACTTTGAATTCA | |
| | GCTACAACTTCGAGGACGTGCCCTTCCACAGCAGCTACGCGCACAGCC | |
| | AGAGCCTGGACCGGCTGATGAACCCTCTCATCGACCAGTACTTGTACT | |
| | ACCTGTCCCGGACTCAAAGCACGGGCGGTACTGCAGGAACTCAGCAGT | |
| | TGCTATTTTCTCAGGCCGGGCCTAACAACATGTCGGCTCAGGCCAAGA | |
| | ACTGGCTACCCGGTCCCTGCTACCGGCAGCAACGTGTCTCCACGACAC | |
| | TGTCGCAGAACAACAACAGCAACTTTGCCTGGACGGGTGCCACCAAGT | |
| | ATCATCTGAATGGCAGAGACTCTCTGGTGAATCCTGGCGTTGCCATGG | |
| | CTACCCACAAGGACGACGAAGAGCGATTTTTTCCATCCAGCGGAGTCT | |
| | TAATGTTTGGGAAACAGGGAGCTGGAAAAGACAACGTGGACTATAGCA | |
| | GCGTGATGCTAACCAGCGAGGAAGAAATAAAGACCACCAACCCAGTGG | |
| | CCACAGAACAGTACGGCGTGGTGGCCGATAACCTGCAACAGCAAAACG | |
| | CCGCTCCTATTGTAGGGGCCGTCAATAGTCAAGGAGCCTTACCTGGCA | |
| | TGGTGTGGCAGAACCGGGACGTGTACCTGCAGGGTCCCATCTGGGCC | |
| | AAGATTCCTCATACGGACGGCAACTTTCATCCCTCGCCGCTGATGGGA | |
| | GGCTTTGGACTGAAGCATCCGCCTCCTCAGATCCTGATTAAAAACACAC | |
| | CTGTTCCCGQGGATCCTCCGACCACCTTCAATCAGGCCAAGCTGGCTT | |
| | CTTTCATCACGCAGTACAGTACCGGCCAGGTCAGCGTGGAGATCGAGT | |
| | GGGAGCTGCAGAAGGAGAACAGCAAACGCTGGAACCCAGAGATTCAG | |
| | TACACTTCCAACTACTACAAATCTACAAATGTGGACTTTGCTGTCAATAC | |
| | TGAGGGTACTTATTCCGAGCCTCGCCCCATTGGCACCCGTTACCTCAC | |
| | CCGTAATCTGTAA | |
| AAVrh74 VP1 | MAADGYLPDWLEDNLSEGIREWWDLKPGAPKPKANQQKQDNGRGLVLP | 11 |
| wild-type | GYKYLGPFNGLDKGEPVNAADAAALEHDKAYDQQLQAGDNPYLRYNHAD | |
| alternative | AEFQERLQEDTSFGGNLGRAVFQAKKRVLEPLGLVESPVKTAPGKKRPVE | |
| reference | PSPQRSPDSSTGIGKKGQQPAKKRLNFGQTGDSESVPDPQPIGEPPAGPS | |
| sequence | GLGSGTMAAGGGAPMADNNEGADGVGSSSGNWHCDSTWLGDRVITTST | |
| (amino acid) | RTWALPTYNNHLYKQISNGTSGGSTNDNTYFGYSTPWGYFDFNRFHCHF | |
| | SPRDWQRLINNNWGFRPKRLNFKLFNIQVKEVTQNEGTKTIANNLTSTIQV | |
| | FTDSEYQLPYVLGSAHQGCLPPFPADVFMIPQYGYLTLNNGSQAVGRSSF | |
| | YCLEYFPSQMLRTGNNFEFSYNFEDVPFHSSYAHSQSLDRLMNPLIDQYL | |
| | YYLSRTQSTGGTAGTQQLLFSQAGPNNMSAQAKNWLPGPCYRQQRVSTT | |
| | LSQNNNSNFAWTGATKYHLNGRDSLVNPGVAMATHKDDEERFFPSSGVL | |
| | MFGKQGAGKDNVDYSSVMLTSEEEIKTTNPVATEQYGVVADNLQQQNAA | |
| | PIVGAVNSQGALPGMVWQNRDVYLQGPIWAKIPHTDGNFHPSPLMGGFG | |
| | LKHPPPQILIKNTPVPADPPTTFTKAKLASFITQYSTGQVSVEIEWELQKENS | |
| | KRWNPEIQYTSNYYKSTNVDFAVNTEGTYSEPRPIGTRYLTRNL | |
| V1 (amino acid) | MAADGYLPDWLEDNLSEGIREWWALKPGAPQPKANQQHQDNARGLVLP | 12 |
| | GYKYLGPGNGLDKGEPVNAADAAALEHDKAYDQQLKAGDNPYLKYNHAD | |
| | AEFQERLKEDTSFGGNLGRAVFQAKKRLLEPLGLVEEAAKTAPGKKRPVE | |
| | QSPQEPDSSAGIGKSGAQPAKKRLNFGQTGDTESVPDPQPIGEPPAAPSG | |
| | VGSLTMASGGGAPVADNNEGADGVGSSSGNWHCDSQWLGDRVITTSTR | |
| | TWALPTYNNHLYKQISNSTSGGSSNDNAYFGYSTPWGYFDFNRFHCHFS | |
| | PRDWQRLINNNWGFRPKRLNFKLFNIQVKEVTDNNGVKTIANNLTSTVQVF | |
| | TDSDYQLPYVLGSAHEGCLPPFPADVFMIPQYGYLTLNDGSQAVGRSSFY | |
| | CLEYFPSQMLRTGNNFQFSYEFENVPFHSSYAHSQSLDRLMNPLIDQYLY | |
| | YLSKTINGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGSLIFG | |
| | KQGTGHDNVDADKVMITNEEEIKTTNPVATEAYGTVATNKQSAYAQAQTG | |
| | ALQSQGVLPGMVWQDRDVYLQGPIWAKIPHTDGNFHPSPLMGGFGMKHP | |
| | PPQILIKNTPVPADPPTAFNKDKLNSFITQYSTGQVSVEIEWELQKENSKRW | |
| | NPEIQYTSNYYKSNNVEFAVNTEGVYSEPRPIGTRYLTRNL | |
| V1 (nucleotide) | ATGGCTGCCGATGGTTATCTTCCAGATTGGCTCGAGGACAACCTTAGT | 13 |
| | GAAGGTATTCGCGAGTGGTGGGCTTTGAAACCTGGAGCCCCTCAACCC | |
| | AAGGCAAATCAACAACATCAAGACAACGCTCGAGGTCTTGTGCTTCCG | |
| | GGTTACAAATACCTTGGACCCGGCAACGGACTCGACAAGGGGGAGCC | |
| | GGTCAACGCAGCAGACGCGGCGGCCCTCGAGCACGACAAGGCCTACG | |
| | ACCAGCAGCTCAAGGCCGGAGACAACCCGTACCTCAAGTACAACCACG | |
| | CCGACGCCGAGTTCCAGGAGCGGCTCAAAGAAGATACGTCTTTTGGGG | |
| | GCAACCTCGGGCGAGCAGTCTTCCAGGCCAAAAAGAGGCTTCTTGAAC | |
| | CTCTTGGTCTGGTTGAGGAAGCGGCTAAGACGGCTCCTGGAAAGAAGA | |
| | GGCCTGTAGAGCAGTCTCCTCAGGAACCGGACTCCTCCGCGGGTATTG | |
| | GCAAATCGGGTGCACAGCCCGCTAAAAAGAGACTCAATTTCGGTCAGA | |
| | CTGGCGACACAGAGTCAGTCCCAGACCCTCAACCAATCGGAGAACCTC | |
| | CCGCAGCCCCCTCAGGTGTGGGATCTCTTACAATGGCTTCAGGTGGTG | |
| | GCGCACCAGTGGCAGACAATAACGAAGGTGCCGATGGAGTGGGTAGT | |
| | TCCTCGGGAAATTGGCATTGCGATTCCCAATGGCTGGGGGACAGAGTC | |
| | ATCACCACCAGCACCCGAACCTGGGCCCTGCCCACCTACAACAATCAC | |
| | CTCTACAAGCAAATCTCCAACAGCACATCTGGAGGATCTTCAAATGACA | |
| | ACGCCTACTTCGGCTACAGCACCCCCTGGGGGTATTTTGACTTCAACA | |
| | GATTCCACTGCCACTTCTCACCACGTGACTGGCAGCGACTCATCAACAA | |
| | CAACTGGGGATTCCGGCCTAAGCGACTCAACTTCAAGCTCTTCAACATT | |
| | CAGGTCAAAGAGGTTACGGACAACAATGGAGTCAAGACCATCGCCAAT | |
| | AACCTTACCAGCACGGTCCAGGTCTTCACGGACTCAGACTATCAGCTC | |
| | CCGTACGTGCTCGGGTCGGCTCACGAGGGCTGCCTCCCGCCGTTCCC | |
| | AGCGGACGTTTTCATGATTCCTCAGTACGGGTATCTGACGCTTAATGAT | |
| | GGAAGCCAGGCCGTGGGTCGTTCGTCCTTTTACTGCCTGGAATATTTC | |
| | CCGTCGCAAATGCTAAGAACGGGTAACAACTTCCAGTTCAGCTACGAG | |
| | TTTGAGAACGTACCTTTCCATAGCAGCTACGCTCACAGCCAAAGCCTGG | |
| | ACCGACTAATGAATCCACTCATCGACCAATACTTGTACTATCTCTCAAAG | |
| | ACTATTAACGGTTCTGGACAGAATCAACAAACGCTAAAATTCAGTGTGG | |
| | CCGGACCCAGCAACATGGCTGTCCAGGGAAGAAACTACATACCTGGAC | |
| | CCAGCTACCGACAACAACGTGTCTCAACCACTGTGACTCAAAACAACAA | |
| | CAGCGAATTTGCTTGGCCTGGAGCTTCTTCTTGGGCTCTCAATGGACGT | |
| | AATAGCTTGATGAATCCTGGACCTGCTATGGCCAGCCACAAAGAAGGA | |
| | GAGGACCGTTTCTTTCCTTTGTCTGGATCTTTAATTTTTGGCAAACAAGG | |
| | AACTGGACACGACAACGTGGATGCGGACAAAGTCATGATAACCAACGA | |
| | AGAAGAAATTAAAACTACTAACCCGGTAGCAACGGAGGCATATGGAAC | |
| | CGTGGCCACAAACAAACAGAGTGCCTATGCACAGGCGCAGACCGGCG | |
| | CATTGCAAAGCCAAGGAGTTCTTCCGGGTATGGTTTGGCAGGACAGAG | |
| | ATGTGTACCTGCAAGGACCCATTTGGGCCAAAATTCCTCACACGGACG | |
| | GCAACTTTCACCCTTCTCCGCTGATGGGAGGGTTTGGAATGAAGCACC | |
| | CGCCTCCTCAGATCCTCATCAAAAACACACCTGTACCTGCGGATCCTCC | |
| | AACGGCCTTCAACAAGGACAAGCTGAACTCTTTCATCACCCAGTATTCT | |
| | ACTGGCCAAGTCAGCGTGGAGATCGAGTGGGAGCTGCAGAAGGAAAA | |
| | CAGCAAGCGCTGGAACCCGGAGATCCAGTACACTTCCAACTATTACAA | |
| | GTCTAATAATGTTGAATTTGCTGTTAATACTGAAGGTGTATATAGTGAAC | |
| | CCCGCCCCATTGGCACCAGATACCTGACTCGTAATCTGTAA | |
| V2 (amino acid) | MAADGYLPDWLEDNLSEGIREWWALKPGAPQPKANQQHQDNARGLVLP | 14 |
| | GYKYLGPGNGLDKGEPVNAADAAALEHDKAYDQQLKAGDNPYLKYNHAD | |
| | AEFQERLKEDTSFGGNLGRAVFQAKKRLLEPLGLVEEAAKTAPGKKRPVE | |
| | QSPQEPDSSAGIGKSGAQPAKKRLNFGQTGDTESVPDPQPIGEPPAAPSG | |
| | VGSLTMASGGGAPVADNNEGADGVGSSSGNWHCDSQWLGDRVITTSTR | |
| | TWALPTYNNHLYKQISNSTSGGSSNDNAYFGYSTPWGYFDFNRFHCHFS | |
| | PRDWQRLINNNWGFRPKRLNFKLFNIQVKEVTDNNGVKTIANNLTSTVQVF | |
| | TDSDYQLPYVLGSAHEGCLPPFPADVFMIPQYGYLTLNDGSQAVGRSSFY | |
| | CLEYFPSQMLRTGNNFQFSYEFENVPFHSSYAHSQSLDRLMNPLIDQYLY | |
| | YLSKTINGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGSLIFG | |
| | KQGTGHNNVDADKVMITNEEEIKTTNPVATEAYGTVATNKQSAYAQAQTG | |
| | ALQSQGVLPGMVWQDRDVYLQGPIWAKIPHTDGNFHPSPLMGGFGMKHP | |
| | PPQILIKNTPVPADPPTAFNKDKLNSFITQYSTGQVSVEIEWELQKENSKRW | |
| | NPEIQYTSNYYKSNNVEFAVNTEGVYSEPRPIGTRYLTRNL | |
| V2 (nucleotide) | ATGGCTGCCGATGGTTATCTTCCAGATTGGCTCGAGGACAACCTTAGT | 15 |
| | GAAGGTATTCGCGAGTGGTGGGCTTTGAAACCTGGAGCCCCTCAACCC | |
| | AAGGCAAATCAACAACATCAAGACAACGCTCGAGGTCTTGTGCTTCCG | |
| | GGTTACAAATACCTTGGACCCGGCAACGGACTCGACAAGGGGGAGCC | |
| | GGTCAACGCAGCAGACGCGGCGGCCCTCGAGCACGACAAGGCCTACG | |
| | ACCAGCAGCTCAAGGCCGGAGACAACCCGTACCTCAAGTACAACCACG | |
| | CCGACGCCGAGTTCCAGGAGCGGCTCAAAGAAGATACGTCTTTTGGGG | |
| | GCAACCTCGGGCGAGCAGTCTTCCAGGCCAAAAAGAGGCTTCTTGAAC | |
| | CTCTTGGTCTGGTTGAGGAAGCGGCTAAGACGGCTCCTGGAAAGAAGA | |
| | GGCCTGTAGAGCAGTCTCCTCAGGAACCGGACTCCTCCGCGGGTATTG | |
| | GCAAATCGGGTGCACAGCCCGCTAAAAAGAGACTCAATTTCGGTCAGA | |
| | CTGGCGACACAGAGTCAGTCCCAGACCCTCAACCAATCGGAGAACCTC | |
| | CCGCAGCCCCCTCAGGTGTGGGATCTCTTACAATGGCTTCAGGTGGTG | |
| | GCGCACCAGTGGCAGACAATAACGAAGGTGCCGATGGAGTGGGTAGT | |
| | TCCTCGGGAAATTGGCATTGCGATTCCCAATGGCTGGGGGACAGAGTC | |
| | ATCACCACCAGCACCCGAACCTGGGCCCTGCCCACCTACAACAATCAC | |
| | CTCTACAAGCAAATCTCCAACAGCACATCTGGAGGATQTTCAAATGACA | |
| | ACGCCTACTTCGGCTACAGCACCCCCTGGGGGTATTTTGACTTCAACA | |
| | GATTCCACTGCCACTTCTCACCACGTGACTGGCAGCGACTCATCAACAA | |
| | CAACTGGGGATTCCGGCCTAAGCGACTCAACTTCAAGCTCTTCAACATT | |
| | CAGGTCAAAGAGGTTACGGACAACAATGGAGTCAAGACCATCGCCAAT | |
| | AACCTTACCAGCACGGTCCAGGTCTTCACGGACTCAGACTATCAGCTC | |
| | CCGTACGTGCTCGGGTCGGCTCACGAGGGCTGCCTCCCGCCGTTCCC | |
| | AGCGGACGTTTTCATGATTCCTCAGTACGGGTATCTGACGCTTAATGAT | |
| | GGAAGCCAGGCCGTGGGTCGTTCGTCCTTTTACTGCCTGGAATATTTC | |
| | CCGTCGCAAATGCTAAGAACGGGTAACAACTTCCAGTTCAGCTACGAG | |
| | TTTGAGAACGTACCTTTCCATAGCAGCTACGCTCACAGCCAAAGCCTGG | |
| | ACCGACTAATGAATCCACTCATCGACCAATACTTGTACTATCTCTCAAAG | |
| | ACTATTAACGGTTCTGGACAGAATCAACAAACGCTAAAATTCAGTGTGG | |
| | CCGGACCCAGCAACATGGCTGTCCAGGGAAGAAACTACATACCTGGAC | |
| | CCAGCTACCGACAACAACGTGTCTCAACCACTGTGACTCAAAACAACAA | |
| | CAGCGAATTTGCTTGGCCTGGAGCTTCTTCTTGGGCTCTCAATGGACGT | |
| | AATAGCTTGATGAATCCTGGACCTGCTATGGCCAGCCACAAAGAAGGA | |
| | GAGGACCGTTTCTTTCCTTTGTCTGGATCTTTAATTTTTGGCAAACAAGG | |
| | AACTGGACATAATAACGTGGATGCGGACAAAGTCATGATAACCAACGAA | |
| | GAAGAAATTAAAACTACTAACCCGGTAGCAACGGAGGCCTATGGAACT | |
| | GTGGCCACAAACAAGCAGAGTGCCTACGCACAGGCGCAGACCGGCGC | |
| | TTTACAAAGCCAAGGAGTCCTTCCGGGTATGGTTTGGCAGGACAGAGA | |
| | TGTGTACCTGCAAGGACCCATTTGGGCCAAAATTCCTCACACGGACGG | |
| | CAACTTTCACCCTTCTCCGCTGATGGGAGGGTTTGGAATGAAGCACCC | |
| | GCCTCCTCAGATCCTCATCAAAAACACACCTGTACCTGCGGATCCTCCA | |
| | ACGGCCTTCAACAAGGACAAGCTGAACTCTTTCATCACCCAGTATTCTA | |
| | CTGGCCAAGTCAGCGTGGAGATCGAGTGGGAGCTGCAGAAGGAAAAC | |
| | AGCAAGCGCTGGAACCCGGAGATCCAGTACACTTCCAACTATTACAAG | |
| | TCTAATAATGTTGAATTTGCTGTTAATACTGAAGGTGTATATAGTGAACC | |
| | CCGCCCCATTGGCACCAGATACCTGACTCGTAATCTGTAA | |
| V3 (amino acid) | MAADGYLPDWLEDNLSEGIREWWALKPGAPQPKANQQHQDNARGLVLP | 16 |
| | GYKYLGPGNGLDKGEPVNAADAAALEHDKAYDQQLKAGDNPYLKYNHAD | |
| | AEFQERLKEDTSFGGNLGRAVFQAKKRLLEPLGLVEEAAKTAPGKKRPVE | |
| | QSPQEPDSSAGIGKSGAQPAKKRLNFGQTGDTESVPDPQPIGEPPAAPSG | |
| | VGSLTMASGGGAPVADNNEGADGVGSSSGNWHCDSQWLGDRVITTSTR | |
| | TWALPTYNNHLYKQISNSTSGGSSNDNAYFGYSTPWGYFDFNRFHCHFS | |
| | PRDWQRLINNNWGFRPKRLNFKLFNIQVKEVTDNNGVKTIANNLTSTVQVF | |
| | TDSDYQLPYVLGSAHEGCLPPFPADVFMIPQYGYLTLNDGSQAVGRSSFY | |
| | CLEYFPSQMLRTGNNFQFSYEFENVPFHSSYAHSQSLDRLMNPLIDQYLY | |
| | YLSKTINGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGSLIFG | |
| | KQGTGHDNVDADKVMITNEEEIKTTNPVATESYGTVATNKQNHTQQAQTG | |
| | ALQSQGVLPGMVWQDRDVYLQGPIWAKIPHTDGNFHPSPLMGGFGMKHP | |
| | PPQILIKNTPVPADPPTAFNKDKLNSFITQYSTGQVSVEIEWELQKENSKRW | |
| | NPEIQYTSNYYKSNNVEFAVNTEGVYSEPRPIGTRYLTRNL | |
| V3 (nucleotide) | ATGGCTGCCGATGGTTATCTTCCAGATTGGCTCGAGGACAACCTTAGT | 17 |
| | GAAGGTATTCGCGAGTGGTGGGCTTTGAAACCTGGAGCCCCTCAACCC | |
| | AAGGCAAATCAACAACATCAAGACAACGCTCGAGGTCTTGTGCTTCCG | |
| | GGTTACAAATACCTTGGACCCGGCAACGGACTCGACAAGGGGGAGCC | |
| | GGTCAACGCAGCAGACGCGGCGGCCCTCGAGCACGACAAGGCCTACG | |
| | ACCAGCAGCTCAAGGCCGGAGACAACCCGTACCTCAAGTACAACCACG | |
| | CCGACGCCGAGTTCCAGGAGCGGCTCAAAGAAGATACGTCTTTTGGGG | |
| | GCAACCTCGGGCGAGCAGTCTTCCAGGCCAAAAAGAGGCTTCTTGAAC | |
| | CTCTTGGTCTGGTTGAGGAAGCGGCTAAGACGGCTCCTGGAAAGAAGA | |
| | GGCCTGTAGAGCAGTCTCCTCAGGAACCGGACTCCTCCGCGGGTATTG | |
| | GCAAATCGGGTGCACAGCCCGCTAAAAAGAGACTCAATTTCGGTCAGA | |
| | CTGGCGACACAGAGTCAGTCCCAGACCCTCAACCAATCGGAGAACCTC | |
| | CCGCAGCCCCCTCAGGTGTGGGATCTCTTACAATGGCTTCAGGTGGTG | |
| | GCGCACCAGTGGCAGACAATAACGAAGGTGCCGATGGAGTGGGTAGT | |
| | TCCTCGGGAAATTGGCATTGCGATTCCCAATGGCTGGGGGACAGAGTC | |
| | ATCACCACCAGCACCCGAACCTGGGCCCTGCCCACCTACAACAATCAC | |
| | CTCTACAAGCAAATCTCCAACAGCACATCTGGAGGATCTTCAAATGACA | |
| | ACGCCTACTTCGGCTACAGCACCCCCTGGGGGTATTTTGACTTCAACA | |
| | GATTCCACTGCCACTTCTCACCACGTGACTGGCAGCGACTCATCAACAA | |
| | CAACTGGGGATTCCGGCCTAAGCGACTCAACTTCAAGCTCTTCAACATT | |
| | CAGGTCAAAGAGGTTACGGACAACAATGGAGTCAAGACCATCGCCAAT | |
| | AACCTTACCAGCACGGTCCAGGTCTTCACGGACTCAGACTATCAGCTC | |
| | CCGTACGTGCTCGGGTCGGCTCACGAGGGCTGCCTCCCGCCGTTCCC | |
| | AGCGGACGTTTTCATGATTCCTCAGTACGGGTATCTGACGCTTAATGAT | |
| | GGAAGCCAGGCCGTGGGTCGTTCGTCCTTTTACTGCCTGGAATATTTC | |
| | CCGTCGCAAATGCTAAGAACGGGTAACAACTTCCAGTTCAGCTACGAG | |
| | TTTGAGAACGTACCTTTCCATAGCAGCTACGCTCACAGCCAAAGCCTGG | |
| | ACCGACTAATGAATCCACTCATCGACCAATACTTGTACTATCTCTCAAAG | |
| | ACTATTAACGGTTCTGGACAGAATCAACAAACGCTAAAATTCAGTGTGG | |
| | CCGGACCCAGCAACATGGCTGTCCAGGGAAGAAACTACATACCTGGAC | |
| | CCAGCTACCGACAACAACGTGTCTCAACCACTGTGACTCAAAACAACAA | |
| | CAGCGAATTTGCTTGGCCTGGAGCTTCTTCTTGGGCTCTCAATGGACGT | |
| | AATAGCTTGATGAATCCTGGACCTGCTATGGCCAGCCACAAAGAAGGA | |
| | GAGGACCGTTTCTTTCCTTTGTCTGGATCTTTAATTTTTGGCAAACAAGG | |
| | AACTGGACATGACAACGTGGATGCGGACAAAGTCATGATAACCAACGA | |
| | AGAAGAAATTAAAACTACTAACCCGGTAGCAACGGAGTCCTATGGAACC | |
| | GTGGCCACAAACAAGCAGAATCATACCCAGCAGGCGCAGACCGGCGC | |
| | CCTACAATCACAAGGAGTCCTTCCGGGTATGGTTTGGCAGGACAGAGA | |
| | TGTGTACCTGCAAGGACCCATTTGGGCCAAAATTCCTCACACGGACGG | |
| | CAACTTTCACCCTTCTCCGCTGATGGGAGGGTTTGGAATGAAGCACCC | |
| | GCCTCCTCAGATCCTCATCAAAAACACACCTGTACCTGCGGATCCTCCA | |
| | ACGGCCTTCAACAAGGACAAGCTGAACTCTTTCATCACCCAGTATTCTA | |
| | CTGGCCAAGTCAGCGTGGAGATCGAGTGGGAGCTGCAGAAGGAAAAC | |
| | AGCAAGCGCTGGAACCCGGAGATCCAGTACACTTCCAACTATTACAAG | |
| | TCTAATAATGTTGAATTTGCTGTTAATACTGAAGGTGTATATAGTGAACC | |
| | CCGCCCCATTGGCACCAGATACCTGACTCGTAATCTGTAA | |
| V4 (amino acid) | MAADGYLPDWLEDNLSEGIREWWALKPGAPQPKANQQHQDNARGLVLP | 18 |
| | GYKYLGPGNGLDKGEPVNAADAAALEHDKAYDQQLKAGDNPYLKYNHAD | |
| | AEFQERLKEDTSFGGNLGRAVFQAKKRLLEPLGLVEEAAKTAPGKKRPVE | |
| | QSPQEPDSSAGIGKSGAQPAKKRLNFGQTGDTESVPDPQPIGEPPAAPSG | |
| | VGSLTMASGGGAPVADNNEGADGVGSSSGNWHCDSQWLGDRVITTSTR | |
| | TWALPTYNNHLYKQISNSTSGGSSNDNAYFGYSTPWGYFDFNRFHCHFS | |
| | PRDWQRLINNNWGFRPKRLNFKLFNIQVKEVTDNNGVKTIANNLTSTVQVF | |
| | TDSDYQLPYVLGSAHEGCLPPFPADVFMIPQYGYLTLNDGSQAVGRSSFY | |
| | CLEYFPSQMLRTGNNFQFSYEFENVPFHSSYAHSQSLDRLMNPLIDQYLY | |
| | YLSKTINGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGSLIFG | |
| | KQGTGRDNVDADKVMITNEEEIKTTNPVATESYGIVATNHQSSNTQANVGA | |
| | LQSQGVLPGMVWQDRDVYLQGPIWAKIPHTDGNFHPSPLMGGFGMKHPP | |
| | PQILIKNTPVPADPPTAFNKDKLNSFITQYSTGQVSVEIEWELQKENSKRWN | |
| | PEIQYTSNYYKSNNVEFAVNTEGVYSEPRPIGTRYLTRNL | |
| V4 (nucleotide) | ATGGCTGCCGATGGTTATCTTCCAGATTGGCTCGAGGACAACCTTAGT | 19 |
| | GAAGGTATTCGCGAGTGGTGGGCTTTGAAACCTGGAGCCCCTCAACCC | |
| | AAGGCAAATCAACAACATCAAGACAACGCTCGAGGTCTTGTGCTTCCG | |
| | GGTTACAAATACCTTGGACCCGGCAACGGACTCGACAAGGGGGAGCC | |
| | GGTCAACGCAGCAGACGCGGCGGCCCTCGAGCACGACAAGGCCTACG | |
| | ACCAGCAGCTCAAGGCCGGAGACAACCCGTACCTCAAGTACAACCACG | |
| | CCGACGCCGAGTTCCAGGAGCGGCTCAAAGAAGATACGTCTTTTGGGG | |
| | GCAACCTCGGGCGAGCAGTCTTCCAGGCCAAAAAGAGGCTTCTTGAAC | |
| | CTCTTGGTCTGGTTGAGGAAGCGGCTAAGACGGCTCCTGGAAAGAAGA | |
| | GGCCTGTAGAGCAGTCTCCTCAGGAACCGGACTCCTCCGCGGGTATTG | |
| | GCAAATCGGGTGCACAGCCCGCTAAAAAGAGACTCAATTTCGGTCAGA | |
| | CTGGCGACACAGAGTCAGTCCCAGACCCTCAACCAATCGGAGAACCTC | |
| | CCGCAGCCCCCTCAGGTGTGGGATCTCTTACAATGGCTTCAGGTGGTG | |
| | GCGCACCAGTGGCAGACAATAACGAAGGTGCCGATGGAGTGGGTAGT | |
| | TCCTCGGGAAATTGGCATTGCGATTCCCAATGGCTGGGGGACAGAGTC | |
| | ATCACCACCAGCACCCGAACCTGGGCCCTGCCCACCTACAACAATCAC | |
| | CTCTACAAGCAAATCTCCAACAGCACATCTGGAGGATCTTCAAATGACA | |
| | ACGCCTACTTCGGCTACAGCACCCCCTGGGGGTATTTTGACTTCAACA | |
| | GATTCCACTGCCACTTCTCACCACGTGACTGGCAGCGACTCATCAACAA | |
| | CAACTGGGGATTCCGGCCTAAGCGACTCAACTTCAAGCTCTTCAACATT | |
| | CAGGTCAAAGAGGTTACGGACAACAATGGAGTCAAGACCATCGCCAAT | |
| | AACCTTACCAGCACGGTCCAGGTCTTCACGGACTCAGACTATCAGCTC | |
| | CCGTACGTGCTCGGGTCGGCTCACGAGGGCTGCCTCCCGCCGTTCCC | |
| | AGCGGACGTTTTCATGATTCCTCAGTACGGGTATCTGACGCTTAATGAT | |
| | GGAAGCCAGGCCGTGGGTCGTTCGTCCTTTTACTGCCTGGAATATTTC | |
| | CCGTCGCAAATGCTAAGAACGGGTAACAACTTCCAGTTCAGCTACGAG | |
| | TTTGAGAACGTACCTTTCCATAGCAGCTACGCTCACAGCCAAAGCCTGG | |
| | ACCGACTAATGAATCCACTCATCGACCAATACTTGTACTATCTCTCAAAG | |
| | ACTATTAACGGTTCTGGACAGAATCAACAAACGCTAAAATTCAGTGTGG | |
| | CCGGACCCAGCAACATGGCTGTCCAGGGAAGAAACTACATACCTGGAC | |
| | CCAGCTACCGACAACAACGTGTCTCAACCACTGTGACTCAAAACAACAA | |
| | CAGCGAATTTGCTTGGCCTGGAGCTTCTTCTTGGGCTCTCAATGGACGT | |
| | AATAGCTTGATGAATCCTGGACCTGCTATGGCCAGCCACAAAGAAGGA | |
| | GAGGACCGTTTCTTTCCTTTGTCTGGATCTTTAATTTTTGGCAAACAAGG | |
| | AACTGGAAGAGACAACGTGGATGCGGACAAAGTCATGATAACCAACGA | |
| | AGAAGAAATTAAAACTACTAACCCGGTAGCAACGGAGTCCTATGGAATA | |
| | GTGGCCACAAACCACCAGAGTAGCAATACGCAGGCGAATGTGGGCGC | |
| | GTTGCAATCGCAAGGAGTGCTTCCGGGTATGGTTTGGCAGGACAGAGA | |
| | TGTGTACCTGCAAGGACCCATTTGGGCCAAAATTCCTCACACGGACGG | |
| | CAACTTTCACCCTTCTCCGCTGATGGGAGGGTTTGGAATGAAGCACCC | |
| | GCCTCCTCAGATCCTCATCAAAAACACACCTGTACCTGCGGATCCTCCA | |
| | ACGGCCTTCAACAAGGACAAGCTGAACTCTTTCATCACCCAGTATTCTA | |
| | CTGGCCAAGTCAGCGTGGAGATCGAGTGGGAGCTGCAGAAGGAAAAC | |
| | AGCAAGCGCTGGAACCCGGAGATCCAGTACACTTCCAACTATTACAAG | |
| | TCTAATAATGTTGAATTTGCTGTTAATACTGAAGGTGTATATAGTGAACC | |
| | CCGCCCCATTGGCACCAGATACCTGACTCGTAATCTGTAA | |
| CBh promoter | CCAACCTGAAAAAAAGTGATTTCAGGCAGGTGCTCCAGGTAATTAAACA | 100 |
| | TTAATACCCCACCAACCAACCATCCCTTAAACCCTTACCTCTTGCTCAG | |
| | CTAATTACAGCCCGGAGGAGAAGGGCCGTCCCGCCCGCTCACCTGTG | |
| | GGAGTAACGCGGTCAGTCAGAGCCGGGGGGGGCGGCGCGAGGCGGC | |
| | GGCGGAGCGGGGCACGGGGCGAAGGCAGCGCGCAGCGACTCCCGCC | |
| | CGCCGCGCGCTTCGCTTTTTATAGGGCCGCCGCCGCCGCCGCCTCGC | |
| | CATAAAAGGAAACTTTCGGAGCGCGCCGCTCTGATTGGCTGCCGCCGC | |
| | ACCTCTCCGCCTCGCCCCGCCCCGCCCCTCGCCCCGCCCCGCCCCGC | |
| | CTGGCGCGCGCCCCCCCCCCCCCCCCGCCCCCATCGCTGCACAAAAT | |
| | AATTAAAAAATAAATAAATACAAAATTGGGGGTGGGGAGGGGGGGGAG | |
| | ATGGGGAGAGTGAAGCAGAACGTGGGGCTCACCTCGACCATGGTAATA | |
| | GCGATGACTAATACGTAGATGTACTGCCAAGTAGGAAAGTCCCATAAG | |
| | GTCATGTACTGGGCACAATGCCAGGCGGGCCATTTACCGTCATTGACG | |
| | TCAATAGGGGGCGTACTTGGCATATGATACACTTGATGTACTGCCAAGT | |
| | GGGCAGTTTACCGTAAATACTCCACCCATTGACGTCAATGGAAAGTCCC | |
| | TATTGGCGTTACTATTGACGTCAATGGGGGGGGGTCGTTGGGCGGTCA | |
| | GCCAGGCGGGCCATTTACCGTAAGTTATGTAACG | |
| Human EF1α | TTGGCTCCGGTGCCCGTCAGTGGGCAGAGCGCACATCGCCCACAGTC | 101 |
| promoter | CCCGAGAAGTIGTGGGGAGGGGTCGGCAATTGAACCGGTGCCTAGAG | |
| | AAGGTGGCGCGGGGTAAACTGGGAAAGTGATGTCGTGTACTGGCTCC | |
| | GCCTTTTTCCCGAGGGTGGGGGAGAACCGTATATAAGTGCAGTAGTCG | |
| | CCGTGAACGTTCTTTTTCGCAACGGGTTTGCCGCCAGAACACAGGTAA | |
| | GTGCCGTGTGTGGTTCCCGCGGGCCTGGCCTCTTTACGGGTTATGGCC | |
| | CTTGCGTGCCTTGAATTACTTCCACCTGGCTGCAGTACGTGATTCTTGA | |
| | TCCCGAGCTTCGGGTTGGAAGTGGGTGGGAGAGTTCGAGGCCTTGCG | |
| | CTTAAGGAGCCCCTTCGCCTCGTGCTTGAGTTGAGGCCTGGCCTGGGC | |
| | GCTGGGGCCGCCGCGTGCGAATCTGGTGGCACCTTCGCGCCTGTCTC | |
| | GCTGCTTTCGATAAGTCTCTAGCCATTTAAAATTTTTGATGACCTGCTGC | |
| | GACGCTTTTTTTCTGGCAAGATAGTCTTGTAAATGCGGGCCAAGATCTG | |
| | CACACTGGTATTTCGGTTTTTGGGGCCGCGGGCGGCGACGGGGCCCG | |
| | TGCGTCCCAGCGCACATGTTCGGCGAGGCGGGGCCTGCGAGCGCGG | |
| | CCACCGAGAATCGGACGGGGGTAGTCTCAAGCTGGCCGGCCTGCTCT | |
| | GGTGCCTGGCCTCGCGCCGCCGTGTATCGCCCCGCCCTGGGCGGCAA | |
| | GGCTGGCCCGGTCGGCACCAGTTGCGTGAGCGGAAAGATGGCCGCTT | |
| | CCCGGCCCTGCTGCAGGGAGCTCAAAATGGAGGACGCGGCGCTCGGG | |
| | AGAGCGGGCGGGTGAGTCACCCACACAAAGGAAAAGGGCCTTTCCGT | |
| | CCTCAGCCGTCGCTTCATGTGACTCCACGGAGTACCGGGCGCCGTCCA | |
| | GGCACCTCGATTAGTTCTCGAGCTTTTGGAGTACGTCGTCTTTAGGTTG | |
| | GGGGGAGGGGTTTTATGCGATGGAGTTTCCCCACACTGAGTGGGTGG | |
| | AGACTGAAGTTAGGCCAGCTTGGCACTTGATGTAATTCTCCTTGGAATT | |
| | TGCCCTTTTTGAGTTTGGATCTTGGTTCATTCTCAAGCCTCAGACAGTG | |
| | GTTCAAAGTTTTTTTCTTCCATTTCAGGTGTCGTGA | |
| hSYN | AGTGCAAGTGGGTTTTAGGACCAGGATGAGGCGGGGTGGGGGTGCCT | 102 |
| | ACCTGACGACCGACCCCGACCCACTGGACAAGCACCCAACCCCCATTC | |
| | CCCAAATTGCGCATCCCCTATCAGAGAGGGGGAGGGGAAACAGGATG | |
| | CGGCGAGGCGCGTGCGCACTGCCAGCTTCAGCACCGCGGACAGTGCC | |
| | TTCGCCCCCGCCTGGCGGCGCGCGCCACCGCCGCCTCAGCACTGAAG | |
| | GCGCGCTGACGTCACTCGCCGGTCCCCCGCAAACTCCCCTTCCCGGC | |
| | CACCTTGGTCGCGTCCGCGCCGCCGCCGGCCCAGCCGGACCGCACCA | |
| | CGCGAGGCGCGAGATAGGGGGGCACGGGCGCGACCATCTGCGCTGC | |
| | GGCGCCGGCGACTCAGCGCTGCCTCAGTCTGCGGTGGGCAGCGGAG | |
| | GAGTCGTGTCGTGCCTGAGAGCGCAG | |
| CNS Targeting | PLNGAVHLY | 103 |
| peptide | | |
| CNS Targeting | IVMNSLK | 104 |
| peptide | | |
| CNS Targeting | RDSPKGW | 105 |
| peptide | | |
| CNS Targeting | YSTDVRM | 106 |
| peptide | | |
| CNS Targeting | RESPRGL | 107 |
| peptide | | |
| CNS Targeting | GNNTRSV | 108 |
| peptide | | |
| CNS Targeting | GNNTRDT | 109 |
| peptide | | |
| CNS Targeting | TNSTRPV | 110 |
| peptide | | |
| Muscle | SLEEEWAQVECEVYGRGCPSGSLDESFYDWFERQL | 111 |
| Targeting | | |
| peptide | | |
| Muscle | TPFYPVGYTVKQPGTCGDGVLQPGEECDDGNPDVSDGCIDCHRAYCGDG | 112 |
| Targeting | YRHQGVEDCDGSDFGYLTCETYLPGSYGDLRCTQYCSIDSTPCRYFT | |
| peptide | | |
| Muscle | RGDLGLS | 113 |
| Targeting | | |
| peptide | | |
| Muscle | RGDLSTP | 114 |
| Targeting | | |
| peptide | | |
| Muscle | SNSRGDYNSL | 115 |
| Targeting | | |
| peptide | | |
| Muscle | ENRRGDFNNT | 116 |
| Targeting | | |
| peptide | | |
| Muscle | SRGDYNSL | 117 |
| Targeting | | |
| peptide | | |
| Muscle | RGDYNSL | 118 |
| Targeting | | |
| peptide | | |
| Muscle | RGDLST | 119 |
| Targeting | | |
| peptide | | |
| Muscle | RGDYVGL | 120 |
| Targeting | | |
| peptide | | |
| Muscle | RGDAVGV | 121 |
| Targeting | | |
| peptide | | |
| Amino acids | AYGTVATNKQSAY | 200 |
| 576-588 of SEQ | | |
| ID NO: 12 and | | |
| SEQ ID NO: 14 | | |
| Amino acids | HDNVDADKVMITNEEEIKTTNPVATEAYGTVATNKQSAYAQAQTGALQSQ | 201 |
| 550-601 of SEQ | GV | |
| ID NO: 12 | | |
| Amino acids | HNNVDADKVMITNEEEIKTTNPVATEAYGTVATNKQSAYAQAQTGALQSQ | 202 |
| 550-601 of SEQ | GV | |
| ID NO: 14 | | |
| Amino acids | TVATNKQNHT | 203 |
| 579-588 of SEQ | | |
| ID NO: 16 | | |
| Amino acids | HDNVDADKVMITNEEEIKTTNPVATESYGTVATNKQNHTQQAQTGALQSQ | 204 |
| 550-601 of SEQ | GV | |
| ID NO: 16 | | |
| Amino acids | IVATNHQSSN | 205 |
| 579-588 of SEQ | | |
| ID NO: 18 | | |
| Amino acids | IVATNHQSSNTQANVGALQSQGV | 206 |
| 579-601 of SEQ | | |
| ID NO: 18 | | |
| V5 (amino acid) | MAADGYLPDWLEDNLSEGIREWWALKPGAPQPKANQQHQDNARGLVLP | 300 |
| | GYKYLGPGNGLDKGEPVNAADAAALEHDKAYDQQLKAGDNPYLKYNHAD | |
| | AEFQERLKEDTSFGGNLGRAVFQAKKRLLEPLGLVEEAAKTAPGKKRPVE | |
| | QSPQEPDSSAGIGKSGAQPAKKRLNFGQTGDTESVPDPQPIGEPPAAPSG | |
| | VGSLTMASGGGAPVADNNEGADGVGSSSGNWHCDSQWLGDRVITTSTR | |
| | TWALPTYNNHLYKQISNSTSGGSSNDNAYFGYSTPWGYFDFNRFHCHFS | |
| | PRDWQRLINNNWGFRPKRLNFKLFNIQVKEVTDNNGVKTIANNLTSTVQVF | |
| | TDSDYQLPYVLGSAHEGCLPPFPADVFMIPQYGYLTLNDGSQAVGRSSFY | |
| | CLEYFPSQMLRTGNNFQFSYEFENVPFHSSYAHSQSLDRLMNPLIDQYLY | |
| | YLSKTINGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGSLIFG | |
| | KQGTGHDNVDADKVMITNEEEIKTTNPVATEAYGTVATNKQSAYAQAQTG | |
| | ALQSQGILPGMVWQDRDVYLQGPIWAKIPHTDGNFHPSPLMGGFGMKHP | |
| | PPQILIKNTPVPADPPTAFNKDKLNSFITQYSTGQVSVEIEWELQKENSKRW | |
| | NPEIQYTSNYYKSNNVEFAVNTEGVYSEPRPIGTRYLTRNL | |
| V6 (amino acid) | MAADGYLPDWLEDNLSEGIREWWALKPGAPQPKANQQHQDNARGLVLP | 301 |
| | GYKYLGPGNGLDKGEPVNAADAAALEHDKAYDQQLKAGDNPYLKYNHAD | |
| | AEFQERLKEDTSFGGNLGRAVFQAKKRLLEPLGLVEEAAKTAPGKKRPVE | |
| | QSPQEPDSSAGIGKSGAQPAKKRLNFGQTGDTESVPDPQPIGEPPAAPSG | |
| | VGSLTMASGGGAPVADNNEGADGVGSSSGNWHCDSQWLGDRVITTSTR | |
| | TWALPTYNNHLYKQISNSTSGGSSNDNAYFGYSTPWGYFDFNRFHCHFS | |
| | PRDWQRLINNNWGFRPKRLNFKLFNIQVKEVTDNNGVKTIANNLTSTVQVF | |
| | TDSDYQLPYVLGSAHEGCLPPFPADVFMIPQYGYLTLNDGSQAVGRSSFY | |
| | CLEYFPSQMLRTGNNFQFSYEFENVPFHSSYAHSQSLDRLMNPLIDQYLY | |
| | YLSKTINGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGSLIFG | |
| | KQGTGHDNVDADKVMITNEEEIKTTNPVATEAYGQVATNKQSAYAQAQTG | |
| | ALQSQGVLPGMVWQDRDVYLQGPIWAKIPHTDGNFHPSPLMGGFGMKHP | |
| | PPQILIKNTPVPADPPTAFNKDKLNSFITQYSTGQVSVEIEWELQKENSKRW | |
| | NPEIQYTSNYYKSNNVEFAVNTEGVYSEPRPIGTRYLTRNL | |
| V7 (amino acid) | MAADGYLPDWLEDNLSEGIREWWALKPGAPQPKANQQHQDNARGLVLP | 302 |
| | GYKYLGPGNGLDKGEPVNAADAAALEHDKAYDQQLKAGDNPYLKYNHAD | |
| | AEFQERLKEDTSFGGNLGRAVFQAKKRLLEPLGLVEEAAKTAPGKKRPVE | |
| | QSPQEPDSSAGIGKSGAQPAKKRLNFGQTGDTESVPDPQPIGEPPAAPSG | |
| | VGSLTMASGGGAPVADNNEGADGVGSSSGNWHCDSQWLGDRVITTSTR | |
| | TWALPTYNNHLYKQISNSTSGGSSNDNAYFGYSTPWGYFDFNRFHCHFS | |
| | PRDWQRLINNNWGFRPKRLNFKLFNIQVKEVTDNNGVKTIANNLTSTVQVF | |
| | TDSDYQLPYVLGSAHEGCLPPFPADVFMIPQYGYLTLNDGSQAVGRSSFY | |
| | CLEYFPSQMLRTGNNFQFSYEFENVPFHSSYAHSQSLDRLMNPLIDQYLY | |
| | YLSKTINGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGSLIFG | |
| | KQGTGHDNVDADKVMITNEEEIKTTNPVATEAYGTVATNHQSAYAQAQTG | |
| | ALQSQGVLPGMVWQDRDVYLQGPIWAKIPHTDGNFHPSPLMGGFGMKHP | |
| | PPQILIKNTPVPADPPTAFNKDKLNSFITQYSTGQVSVEIEWELQKENSKRW | |
| | NPEIQYTSNYYKSNNVEFAVNTEGVYSEPRPIGTRYLTRNL | |
| V8 (amino acid) | MAADGYLPDWLEDNLSEGIREWWALKPGAPQPKANQQHQDNARGLVLP | 303 |
| | GYKYLGPGNGLDKGEPVNAADAAALEHDKAYDQQLKAGDNPYLKYNHAD | |
| | AEFQERLKEDTSFGGNLGRAVFQAKKRLLEPLGLVEEAAKTAPGKKRPVE | |
| | QSPQEPDSSAGIGKSGAQPAKKRLNFGQTGDTESVPDPQPIGEPPAAPSG | |
| | VGSLTMASGGGAPVADNNEGADGVGSSSGNWHCDSQWLGDRVITTSTR | |
| | TWALPTYNNHLYKQISNSTSGGSSNDNAYFGYSTPWGYFDFNRFHCHFS | |
| | PRDWQRLINNNWGFRPKRLNFKLFNIQVKEVTDNNGVKTIANNLTSTVQVF | |
| | TDSDYQLPYVLGSAHEGCLPPFPADVFMIPQYGYLTLNDGSQAVGRSSFY | |
| | CLEYFPSQMLRTGNNFQFSYEFENVPFHSSYAHSQSLDRLMNPLIDQYLY | |
| | YLSKTINGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGSLIFG | |
| | KQGTGRDNVDADKVMITNEEEIKTTNPVATEAYGTVATNKQSAYAQAQTG | |
| | ALQSQGVLPGMVWQDRDVYLQGPIWAKIPHTDGNFHPSPLMGGFGMKHP | |
| | PPQILIKNTPVPADPPTAFNKDKLNSFITQYSTGQVSVEIEWELQKENSKRW | |
| | NPEIQYTSNYYKSNNVEFAVNTEGVYSEPRPIGTRYLTRNL | |
| V9 (amino acid) | MAADGYLPDWLEDNLSEGIREWWALKPGAPQPKANQQHQDNARGLVLP | 304 |
| | GYKYLGPGNGLDKGEPVNAADAAALEHDKAYDQQLKAGDNPYLKYNHAD | |
| | AEFQERLKEDTSFGGNLGRAVFQAKKRLLEPLGLVEEAAKTAPGKKRPVE | |
| | QSPQEPDSSAGIGKSGAQPAKKRLNFGQTGDTESVPDPQPIGEPPAAPSG | |
| | VGSLTMASGGGAPVADNNEGADGVGSSSGNWHCDSQWLGDRVITTSTR | |
| | TWALPTYNNHLYKQISNSTSGGSSNDNAYFGYSTPWGYFDFNRFHCHFS | |
| | PRDWQRLINNNWGFRPKRLNFKLFNIQVKEVTDNNGVKTIANNLTSTVQVF | |
| | TDSDYQLPYVLGSAHEGCLPPFPADVFMIPQYGYLTLNDGSQAVGRSSFY | |
| | CLEYFPSQMLRTGNNFQFSYEFENVPFHSSYAHSQSLDRLMNPLIDQYLY | |
| | YLSKTINGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGSLIFG | |
| | KQGTGHDNVDADKVMITNEEEIKTTNPVATESYGTVATNKQSAYAQAQTG | |
| | ALQSQGVLPGMVWQDRDVYLQGPIWAKIPHTDGNFHPSPLMGGFGMKHP | |
| | PPQILIKNTPVPADPPTAFNKDKLNSFITQYSTGQVSVEIEWELQKENSKRW | |
| | NPEIQYTSNYYKSNNVEFAVNTEGVYSEPRPIGTRYLTRNL | |
| V10 (amino | MAADGYLPDWLEDNLSEGIREWWALKPGAPQPKANQQHQDNARGLVLP | 305 |
| acid) | GYKYLGPGNGLDKGEPVNAADAAALEHDKAYDQQLKAGDNPYLKYNHAD | |
| | AEFQERLKEDTSFGGNLGRAVFQAKKRLLEPLGLVEEAAKTAPGKKRPVE | |
| | QSPQEPDSSAGIGKSGAQPAKKRLNFGQTGDTESVPDPQPIGEPPAAPSG | |
| | VGSLTMASGGGAPVADNNEGADGVGSSSGNWHCDSQWLGDRVITTSTR | |
| | TWALPTYNNHLYKQISNSTSGGSSNDNAYFGYSTPWGYFDFNRFHCHFS | |
| | PRDWQRLINNNWGFRPKRLNFKLFNIQVKEVTDNNGVKTIANNLTSTVQVF | |
| | TDSDYQLPYVLGSAHEGCLPPFPADVFMIPQYGYLTLNDGSQAVGRSSFY | |
| | CLEYFPSQMLRTGNNFQFSYEFENVPFHSSYAHSQSLDRLMNPLIDQYLY | |
| | YLSKTINGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGSLIFG | |
| | KQGTGHDNVDADKVMITNEEEIKTTNPVATEAYGTVATNKQSAYAQAQTG | |
| | WLQSQGVLPGMVWQDRDVYLQGPIWAKIPHTDGNFHPSPLMGGFGMKH | |
| | PPPQILIKNTPVPADPPTAFNKDKLNSFITQYSTGQVSVEIEWELQKENSKR | |
| | WNPEIQYTSNYYKSNNVEFAVNTEGVYSEPRPIGTRYLTRNL | |
| V11 (amino | MAADGYLPDWLEDNLSEGIREWWALKPGAPQPKANQQHQDNARGLVLP | 306 |
| acid) | GYKYLGPGNGLDKGEPVNAADAAALEHDKAYDQQLKAGDNPYLKYNHAD | |
| | AEFQERLKEDTSFGGNLGRAVFQAKKRLLEPLGLVEEAAKTAPGKKRPVE | |
| | QSPQEPDSSAGIGKSGAQPAKKRLNFGQTGDTESVPDPQPIGEPPAAPSG | |
| | VGSLTMASGGGAPVADNNEGADGVGSSSGNWHCDSQWLGDRVITTSTR | |
| | TWALPTYNNHLYKQISNSTSGGSSNDNAYFGYSTPWGYFDFNRFHCHFS | |
| | PRDWQRLINNNWGFRPKRLNFKLFNIQVKEVTDNNGVKTIANNLTSTVQVF | |
| | TDSDYQLPYVLGSAHEGCLPPFPADVFMIPQYGYLTLNDGSQAVGRSSFY | |
| | CLEYFPSQMLRTGNNFQFSYEFENVPFHSSYAHSQSLDRLMNPLIDQYLY | |
| | YLSKTINGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGSLIFG | |
| | KQGTGHDNVDADKVMITNEEEIKTTNPVATEAYGTVATNKQSAYAQAQTG | |
| | ALQNQGVLPGMVWQDRDVYLQGPIWAKIPHTDGNFHPSPLMGGFGMKH | |
| | PPPQILIKNTPVPADPPTAFNKDKLNSFITQYSTGQVSVEIEWELQKENSKR | |
| | WNPEIQYTSNYYKSNNVEFAVNTEGVYSEPRPIGTRYLTRNL | |
| V12 (amino | MAADGYLPDWLEDNLSEGIREWWALKPGAPQPKANQQHQDNARGLVLP | 307 |
| acid) | GYKYLGPGNGLDKGEPVNAADAAALEHDKAYDQQLKAGDNPYLKYNHAD | |
| | AEFQERLKEDTSFGGNLGRAVFQAKKRLLEPLGLVEEAAKTAPGKKRPVE | |
| | QSPQEPDSSAGIGKSGAQPAKKRLNFGQTGDTESVPDPQPIGEPPAAPSG | |
| | VGSLTMASGGGAPVADNNEGADGVGSSSGNWHCDSQWLGDRVITTSTR | |
| | TWALPTYNNHLYKQISNSTSGGSSNDNAYFGYSTPWGYFDFNRFHCHFS | |
| | PRDWQRLINNNWGFRPKRLNFKLFNIQVKEVTDNNGVKTIANNLTSTVQVF | |
| | TDSDYQLPYVLGSAHEGCLPPFPADVFMIPQYGYLTLNDGSQAVGRSSFY | |
| | CLEYFPSQMLRTGNNFQFSYEFENVPFHSSYAHSQSLDRLMNPLIDQYLY | |
| | YLSKTINGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGSLIFG | |
| | KQGTGHDNVDADKVMITNEEEIKTTNPVATEAYGTVATNKQSAYAQAQTG | |
| | AVQSQGVLPGMVWQDRDVYLQGPIWAKIPHTDGNFHPSPLMGGFGMKH | |
| | PPPQILIKNTPVPADPPTAFNKDKLNSFITQYSTGQVSVEIEWELQKENSKR | |
| | WNPEIQYTSNYYKSNNVEFAVNTEGVYSEPRPIGTRYLTRNL | |
| V13 (amino | MAADGYLPDWLEDNLSEGIREWWALKPGAPQPKANQQHQDNARGLVLP | 308 |
| acid) | GYKYLGPGNGLDKGEPVNAADAAALEHDKAYDQQLKAGDNPYLKYNHAD | |
| | AEFQERLKEDTSFGGNLGRAVFQAKKRLLEPLGLVEEAAKTAPGKKRPVE | |
| | QSPQEPDSSAGIGKSGAQPAKKRLNFGQTGDTESVPDPQPIGEPPAAPSG | |
| | VGSLTMASGGGAPVADNNEGADGVGSSSGNWHCDSQWLGDRVITTSTR | |
| | TWALPTYNNHLYKQISNSTSGGSSNDNAYFGYSTPWGYFDFNRFHCHFS | |
| | PRDWQRLINNNWGFRPKRLNFKLFNIQVKEVTDNNGVKTIANNLTSTVQVF | |
| | TDSDYQLPYVLGSAHEGCLPPFPADVFMIPQYGYLTLNDGSQAVGRSSFY | |
| | CLEYFPSQMLRTGNNFQFSYEFENVPFHSSYAHSQSLDRLMNPLIDQYLY | |
| | YLSKTINGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGSLIFG | |
| | KQGTGHDNVDADKVMITNEEEIKTTNPVATEAYGTVATNKQSAQAQAQTG | |
| | ALQSQGVLPGMVWQDRDVYLQGPIWAKIPHTDGNFHPSPLMGGFGMKHP | |
| | PPQILIKNTPVPADPPTAFNKDKLNSFITQYSTGQVSVEIEWELQKENSKRW | |
| | NPEIQYTSNYYKSNNVEFAVNTEGVYSEPRPIGTRYLTRNL | |
| V14 (amino | MAADGYLPDWLEDNLSEGIREWWALKPGAPQPKANQQHQDNARGLVLP | 309 |
| acid) | GYKYLGPGNGLDKGEPVNAADAAALEHDKAYDQQLKAGDNPYLKYNHAD | |
| | AEFQERLKEDTSFGGNLGRAVFQAKKRLLEPLGLVEEAAKTAPGKKRPVE | |
| | QSPQEPDSSAGIGKSGAQPAKKRLNFGQTGDTESVPDPQPIGEPPAAPSG | |
| | VGSLTMASGGGAPVADNNEGADGVGSSSGNWHCDSQWLGDRVITTSTR | |
| | TWALPTYNNHLYKQISNSTSGGSSNDNAYFGYSTPWGYFDFNRFHCHFS | |
| | PRDWQRLINNNWGFRPKRLNFKLFNIQVKEVTDNNGVKTIANNLTSTVQVF | |
| | TDSDYQLPYVLGSAHEGCLPPFPADVFMIPQYGYLTLNDGSQAVGRSSFY | |
| | CLEYFPSQMLRTGNNFQFSYEFENVPFHSSYAHSQSLDRLMNPLIDQYLY | |
| | YLSKTINGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGSLIFG | |
| | KQGTGHDNVDADKVMITNEEEIKTTNPVATESYGQVATNKQSAYAQAQTG | |
| | ALQSQGVLPGMVWQDRDVYLQGPIWAKIPHTDGNFHPSPLMGGFGMKHP | |
| | PPQILIKNTPVPADPPTAFNKDKLNSFITQYSTGQVSVEIEWELQKENSKRW | |
| | NPEIQYTSNYYKSNNVEFAVNTEGVYSEPRPIGTRYLTRNL | |
| V15 (amino | MAADGYLPDWLEDNLSEGIREWWALKPGAPQPKANQQHQDNARGLVLP | 310 |
| acid) | GYKYLGPGNGLDKGEPVNAADAAALEHDKAYDQQLKAGDNPYLKYNHAD | |
| | AEFQERLKEDTSFGGNLGRAVFQAKKRLLEPLGLVEEAAKTAPGKKRPVE | |
| | QSPQEPDSSAGIGKSGAQPAKKRLNFGQTGDTESVPDPQPIGEPPAAPSG | |
| | VGSLTMASGGGAPVADNNEGADGVGSSSGNWHCDSQWLGDRVITTSTR | |
| | TWALPTYNNHLYKQISNSTSGGSSNDNAYFGYSTPWGYFDFNRFHCHFS | |
| | PRDWQRLINNNWGFRPKRLNFKLFNIQVKEVTDNNGVKTIANNLTSTVQVF | |
| | TDSDYQLPYVLGSAHEGCLPPFPADVFMIPQYGYLTLNDGSQAVGRSSFY | |
| | CLEYFPSQMLRTGNNFQFSYEFENVPFHSSYAHSQSLDRLMNPLIDQYLY | |
| | YLSKTINGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGSLIFG | |
| | KQGTGHDNVDADKVMITNEEEIKTTNPVATEAYGQVATNKQSAYAQAQTG | |
| | ALQNQGVLPGMVWQDRDVYLQGPIWAKIPHTDGNFHPSPLMGGFGMKH | |
| | PPPQILIKNTPVPADPPTAFNKDKLNSFITQYSTGQVSVEIEWELQKENSKR | |
| | WNPEIQYTSNYYKSNNVEFAVNTEGVYSEPRPIGTRYLTRNL | |
| V16 (amino | MAADGYLPDWLEDNLSEGIREWWALKPGAPQPKANQQHQDNARGLVLP | 311 |
| acid) | GYKYLGPGNGLDKGEPVNAADAAALEHDKAYDQQLKAGDNPYLKYNHAD | |
| | AEFQERLKEDTSFGGNLGRAVFQAKKRLLEPLGLVEEAAKTAPGKKRPVE | |
| | QSPQEPDSSAGIGKSGAQPAKKRLNFGQTGDTESVPDPQPIGEPPAAPSG | |
| | VGSLTMASGGGAPVADNNEGADGVGSSSGNWHCDSQWLGDRVITTSTR | |
| | TWALPTYNNHLYKQISNSTSGGSSNDNAYFGYSTPWGYFDFNRFHCHFS | |
| | PRDWQRLINNNWGFRPKRLNFKLFNIQVKEVTDNNGVKTIANNLTSTVQVF | |
| | TDSDYQLPYVLGSAHEGCLPPFPADVFMIPQYGYLTLNDGSQAVGRSSFY | |
| | CLEYFPSQMLRTGNNFQFSYEFENVPFHSSYAHSQSLDRLMNPLIDQYLY | |
| | YLSKTINGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGSLIFG | |
| | KQGTGHDNVDADKVMITNEEEIKTTNPVATEAYGTVATNHQSAYAQAQTG | |
| | AVQSQGVLPGMVWQDRDVYLQGPIWAKIPHTDGNFHPSPLMGGFGMKH | |
| | PPPQILIKNTPVPADPPTAFNKDKLNSFITQYSTGQVSVEIEWELQKENSKR | |
| | WNPEIQYTSNYYKSNNVEFAVNTEGVYSEPRPIGTRYLTRNL | |
| V17 (amino | MAADGYLPDWLEDNLSEGIREWWALKPGAPQPKANQQHQDNARGLVLP | 312 |
| acid) | GYKYLGPGNGLDKGEPVNAADAAALEHDKAYDQQLKAGDNPYLKYNHAD | |
| | AEFQERLKEDTSFGGNLGRAVFQAKKRLLEPLGLVEEAAKTAPGKKRPVE | |
| | QSPQEPDSSAGIGKSGAQPAKKRLNFGQTGDTESVPDPQPIGEPPAAPSG | |
| | VGSLTMASGGGAPVADNNEGADGVGSSSGNWHCDSQWLGDRVITTSTR | |
| | TWALPTYNNHLYKQISNSTSGGSSNDNAYFGYSTPWGYFDFNRFHCHFS | |
| | PRDWQRLINNNWGFRPKRLNFKLFNIQVKEVTDNNGVKTIANNLTSTVQVF | |
| | TDSDYQLPYVLGSAHEGCLPPFPADVFMIPQYGYLTLNDGSQAVGRSSFY | |
| | CLEYFPSQMLRTGNNFQFSYEFENVPFHSSYAHSQSLDRLMNPLIDQYLY | |
| | YLSKTINGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGSLIFG | |
| | KQGTGHDNVDADKVMITNEEEIKTTNPVATESYGTVATNHQSAYAQAQTG | |
| | ALQSQGVLPGMVWQDRDVYLQGPIWAKIPHTDGNFHPSPLMGGFGMKHP | |
| | PPQILIKNTPVPADPPTAFNKDKLNSFITQYSTGQVSVEIEWELQKENSKRW | |
| | NPEIQYTSNYYKSNNVEFAVNTEGVYSEPRPIGTRYLTRNL | |
| V18 (amino | MAADGYLPDWLEDNLSEGIREWWALKPGAPQPKANQQHQDNARGLVLP | 313 |
| acid) | GYKYLGPGNGLDKGEPVNAADAAALEHDKAYDQQLKAGDNPYLKYNHAD | |
| | AEFQERLKEDTSFGGNLGRAVFQAKKRLLEPLGLVEEAAKTAPGKKRPVE | |
| | QSPQEPDSSAGIGKSGAQPAKKRLNFGQTGDTESVPDPQPIGEPPAAPSG | |
| | VGSLTMASGGGAPVADNNEGADGVGSSSGNWHCDSQWLGDRVITTSTR | |
| | TWALPTYNNHLYKQISNSTSGGSSNDNAYFGYSTPWGYFDFNRFHCHFS | |
| | PRDWQRLINNNWGFRPKRLNFKLFNIQVKEVTDNNGVKTIANNLTSTVQVF | |
| | TDSDYQLPYVLGSAHEGCLPPFPADVFMIPQYGYLTLNDGSQAVGRSSFY | |
| | CLEYFPSQMLRTGNNFQFSYEFENVPFHSSYAHSQSLDRLMNPLIDQYLY | |
| | YLSKTINGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGSLIFG | |
| | KQGTGHDNVDADKVMITNEEEIKTTNPVATEAYGTVATNKQSAQAQAQTG | |
| | ALQSQGILPGMVWQDRDVYLQGPIWAKIPHTDGNFHPSPLMGGFGMKHP | |
| | PPQILIKNTPVPADPPTAFNKDKLNSFITQYSTGQVSVEIEWELQKENSKRW | |
| | NPEIQYTSNYYKSNNVEFAVNTEGVYSEPRPIGTRYLTRNL | |
| V19 (amino | MAADGYLPDWLEDNLSEGIREWWALKPGAPQPKANQQHQDNARGLVLP | 314 |
| acid) | GYKYLGPGNGLDKGEPVNAADAAALEHDKAYDQQLKAGDNPYLKYNHAD | |
| | AEFQERLKEDTSFGGNLGRAVFQAKKRLLEPLGLVEEAAKTAPGKKRPVE | |
| | QSPQEPDSSAGIGKSGAQPAKKRLNFGQTGDTESVPDPQPIGEPPAAPSG | |
| | VGSLTMASGGGAPVADNNEGADGVGSSSGNWHCDSQWLGDRVITTSTR | |
| | TWALPTYNNHLYKQISNSTSGGSSNDNAYFGYSTPWGYFDFNRFHCHFS | |
| | PRDWQRLINNNWGFRPKRLNFKLFNIQVKEVTDNNGVKTIANNLTSTVQVF | |
| | TDSDYQLPYVLGSAHEGCLPPFPADVFMIPQYGYLTLNDGSQAVGRSSFY | |
| | CLEYFPSQMLRTGNNFQFSYEFENVPFHSSYAHSQSLDRLMNPLIDQYLY | |
| | YLSKTINGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGSLIFG | |
| | KQGTGRDNVDADKVMITNEEEIKTTNPVATEAYGTVATNKQSAYAQAQTG | |
| | ALQSQGILPGMVWQDRDVYLQGPIWAKIPHTDGNFHPSPLMGGFGMKHP | |
| | PPQILIKNTPVPADPPTAFNKDKLNSFITQYSTGQVSVEIEWELQKENSKRW | |
| | NPEIQYTSNYYKSNNVEFAVNTEGVYSEPRPIGTRYLTRNL | |
| V20 (amino | MAADGYLPDWLEDNLSEGIREWWALKPGAPQPKANQQHQDNARGLVLP | 315 |
| acid) | GYKYLGPGNGLDKGEPVNAADAAALEHDKAYDQQLKAGDNPYLKYNHAD | |
| | AEFQERLKEDTSFGGNLGRAVFQAKKRLLEPLGLVEEAAKTAPGKKRPVE | |
| | QSPQEPDSSAGIGKSGAQPAKKRLNFGQTGDTESVPDPQPIGEPPAAPSG | |
| | VGSLTMASGGGAPVADNNEGADGVGSSSGNWHCDSQWLGDRVITTSTR | |
| | TWALPTYNNHLYKQISNSTSGGSSNDNAYFGYSTPWGYFDFNRFHCHFS | |
| | PRDWQRLINNNWGFRPKRLNFKLFNIQVKEVTDNNGVKTIANNLTSTVQVF | |
| | TDSDYQLPYVLGSAHEGCLPPFPADVFMIPQYGYLTLNDGSQAVGRSSFY | |
| | CLEYFPSQMLRTGNNFQFSYEFENVPFHSSYAHSQSLDRLMNPLIDQYLY | |
| | YLSKTINGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGSLIFG | |
| | KQGTGRDNVDADKVMITNEEEIKTTNPVATEAYGQVATNKQSAYAQAQTG | |
| | ALQSQGVLPGMVWQDRDVYLQGPIWAKIPHTDGNFHPSPLMGGFGMKHP | |
| | PPQILIKNTPVPADPPTAFNKDKLNSFITQYSTGQVSVEIEWELQKENSKRW | |
| | NPEIQYTSNYYKSNNVEFAVNTEGVYSEPRPIGTRYLTRNL | |
| V21 (amino | MAADGYLPDWLEDNLSEGIREWWALKPGAPQPKANQQHQDNARGLVLP | 316 |
| acid) | GYKYLGPGNGLDKGEPVNAADAAALEHDKAYDQQLKAGDNPYLKYNHAD | |
| | AEFQERLKEDTSFGGNLGRAVFQAKKRLLEPLGLVEEAAKTAPGKKRPVE | |
| | QSPQEPDSSAGIGKSGAQPAKKRLNFGQTGDTESVPDPQPIGEPPAAPSG | |
| | VGSLTMASGGGAPVADNNEGADGVGSSSGNWHCDSQWLGDRVITTSTR | |
| | TWALPTYNNHLYKQISNSTSGGSSNDNAYFGYSTPWGYFDFNRFHCHFS | |
| | PRDWQRLINNNWGFRPKRLNFKLFNIQVKEVTDNNGVKTIANNLTSTVQVF | |
| | TDSDYQLPYVLGSAHEGCLPPFPADVFMIPQYGYLTLNDGSQAVGRSSFY | |
| | CLEYFPSQMLRTGNNFQFSYEFENVPFHSSYAHSQSLDRLMNPLIDQYLY | |
| | YLSKTINGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGSLIFG | |
| | KQGTGRDNVDADKVMITNEEEIKTTNPVATESYGTVATNKQSAYAQAQTG | |
| | ALQSQGVLPGMVWQDRDVYLQGPIWAKIPHTDGNFHPSPLMGGFGMKHP | |
| | PPQILIKNTPVPADPPTAFNKDKLNSFITQYSTGQVSVEIEWELQKENSKRW | |
| | NPEIQYTSNYYKSNNVEFAVNTEGVYSEPRPIGTRYLTRNL | |
| V22 (amino | MAADGYLPDWLEDNLSEGIREWWALKPGAPQPKANQQHQDNARGLVLP | 317 |
| acid) | GYKYLGPGNGLDKGEPVNAADAAALEHDKAYDQQLKAGDNPYLKYNHAD | |
| | AEFQERLKEDTSFGGNLGRAVFQAKKRLLEPLGLVEEAAKTAPGKKRPVE | |
| | QSPQEPDSSAGIGKSGAQPAKKRLNFGQTGDTESVPDPQPIGEPPAAPSG | |
| | VGSLTMASGGGAPVADNNEGADGVGSSSGNWHCDSQWLGDRVITTSTR | |
| | TWALPTYNNHLYKQISNSTSGGSSNDNAYFGYSTPWGYFDFNRFHCHFS | |
| | PRDWQRLINNNWGFRPKRLNFKLFNIQVKEVTDNNGVKTIANNLTSTVQVF | |
| | TDSDYQLPYVLGSAHEGCLPPFPADVFMIPQYGYLTLNDGSQAVGRSSFY | |
| | CLEYFPSQMLRTGNNFQFSYEFENVPFHSSYAHSQSLDRLMNPLIDQYLY | |
| | YLSKTINGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGSLIFG | |
| | KQGTGRDNVDADKVMITNEEEIKTTNPVATEAYGTVATNKQSAYAQAQTG | |
| | AVQSQGVLPGMVWQDRDVYLQGPIWAKIPHTDGNFHPSPLMGGFGMKH | |
| | PPPQILIKNTPVPADPPTAFNKDKLNSFITQYSTGQVSVEIEWELQKENSKR | |
| | WNPEIQYTSNYYKSNNVEFAVNTEGVYSEPRPIGTRYLTRNL | |
| V23 (amino | MAADGYLPDWLEDNLSEGIREWWALKPGAPQPKANQQHQDNARGLVLP | 318 |
| acid) | GYKYLGPGNGLDKGEPVNAADAAALEHDKAYDQQLKAGDNPYLKYNHAD | |
| | AEFQERLKEDTSFGGNLGRAVFQAKKRLLEPLGLVEEAAKTAPGKKRPVE | |
| | QSPQEPDSSAGIGKSGAQPAKKRLNFGQTGDTESVPDPQPIGEPPAAPSG | |
| | VGSLTMASGGGAPVADNNEGADGVGSSSGNWHCDSQWLGDRVITTSTR | |
| | TWALPTYNNHLYKQISNSTSGGSSNDNAYFGYSTPWGYFDFNRFHCHFS | |
| | PRDWQRLINNNWGFRPKRLNFKLFNIQVKEVTDNNGVKTIANNLTSTVQVF | |
| | TDSDYQLPYVLGSAHEGCLPPFPADVFMIPQYGYLTLNDGSQAVGRSSFY | |
| | CLEYFPSQMLRTGNNFQFSYEFENVPFHSSYAHSQSLDRLMNPLIDQYLY | |
| | YLSKTINGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGSLIFG | |
| | KQGTGHDNVDADKVMITNEEEIKTTNPVATEAYGQVATNHQSAYAQAQTG | |
| | ALQSQGVLPGMVWQDRDVYLQGPIWAKIPHTDGNFHPSPLMGGFGMKHP | |
| | PPQILIKNTPVPADPPTAFNKDKLNSFITQYSTGQVSVEIEWELQKENSKRW | |
| | NPEIQYTSNYYKSNNVEFAVNTEGVYSEPRPIGTRYLTRNL | |
| V24 (amino | MAADGYLPDWLEDNLSEGIREWWALKPGAPQPKANQQHQDNARGLVLP | 319 |
| acid) | GYKYLGPGNGLDKGEPVNAADAAALEHDKAYDQQLKAGDNPYLKYNHAD | |
| | AEFQERLKEDTSFGGNLGRAVFQAKKRLLEPLGLVEEAAKTAPGKKRPVE | |
| | QSPQEPDSSAGIGKSGAQPAKKRLNFGQTGDTESVPDPQPIGEPPAAPSG | |
| | VGSLTMASGGGAPVADNNEGADGVGSSSGNWHCDSQWLGDRVITTSTR | |
| | TWALPTYNNHLYKQISNSTSGGSSNDNAYFGYSTPWGYFDFNRFHCHFS | |
| | PRDWQRLINNNWGFRPKRLNFKLFNIQVKEVTDNNGVKTIANNLTSTVQVF | |
| | TDSDYQLPYVLGSAHEGCLPPFPADVFMIPQYGYLTLNDGSQAVGRSSFY | |
| | CLEYFPSQMLRTGNNFQFSYEFENVPFHSSYAHSQSLDRLMNPLIDQYLY | |
| | YLSKTINGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGSLIFG | |
| | KQGTGHDNVDADKVMITNEEEIKTTNPVATEAYGTVATNKQSAYAQAQTG | |
| | AVQNQGVLPGMVWQDRDVYLQGPIWAKIPHTDGNFHPSPLMGGFGMKH | |
| | PPPQILIKNTPVPADPPTAFNKDKLNSFITQYSTGQVSVEIEWELQKENSKR | |
| | WNPEIQYTSNYYKSNNVEFAVNTEGVYSEPRPIGTRYLTRNL | |
| V25 (amino | MAADGYLPDWLEDNLSEGIREWWALKPGAPQPKANQQHQDNARGLVLP | 320 |
| acid) | GYKYLGPGNGLDKGEPVNAADAAALEHDKAYDQQLKAGDNPYLKYNHAD | |
| | AEFQERLKEDTSFGGNLGRAVFQAKKRLLEPLGLVEEAAKTAPGKKRPVE | |
| | QSPQEPDSSAGIGKSGAQPAKKRLNFGQTGDTESVPDPQPIGEPPAAPSG | |
| | VGSLTMASGGGAPVADNNEGADGVGSSSGNWHCDSQWLGDRVITTSTR | |
| | TWALPTYNNHLYKQISNSTSGGSSNDNAYFGYSTPWGYFDFNRFHCHFS | |
| | PRDWQRLINNNWGFRPKRLNFKLFNIQVKEVTDNNGVKTIANNLTSTVQVF | |
| | TDSDYQLPYVLGSAHEGCLPPFPADVFMIPQYGYLTLNDGSQAVGRSSFY | |
| | CLEYFPSQMLRTGNNFQFSYEFENVPFHSSYAHSQSLDRLMNPLIDQYLY | |
| | YLSKTINGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGSLIFG | |
| | KQGTGHDNVDADKVMITNEEEIKTTNPVATEAYGTVATNHQSAYAQAQTG | |
| | ALQSQGILPGMVWQDRDVYLQGPIWAKIPHTDGNFHPSPLMGGFGMKHP | |
| | PPQILIKNTPVPADPPTAFNKDKLNSFITQYSTGQVSVEIEWELQKENSKRW | |
| | NPEIQYTSNYYKSNNVEFAVNTEGVYSEPRPIGTRYLTRNL | |
| V26 (amino | MAADGYLPDWLEDNLSEGIREWWALKPGAPQPKANQQHQDNARGLVLP | 321 |
| acid) | GYKYLGPGNGLDKGEPVNAADAAALEHDKAYDQQLKAGDNPYLKYNHAD | |
| | AEFQERLKEDTSFGGNLGRAVFQAKKRLLEPLGLVEEAAKTAPGKKRPVE | |
| | QSPQEPDSSAGIGKSGAQPAKKRLNFGQTGDTESVPDPQPIGEPPAAPSG | |
| | VGSLTMASGGGAPVADNNEGADGVGSSSGNWHCDSQWLGDRVITTSTR | |
| | TWALPTYNNHLYKQISNSTSGGSSNDNAYFGYSTPWGYFDFNRFHCHFS | |
| | PRDWQRLINNNWGFRPKRLNFKLFNIQVKEVTDNNGVKTIANNLTSTVQVF | |
| | TDSDYQLPYVLGSAHEGCLPPFPADVFMIPQYGYLTLNDGSQAVGRSSFY | |
| | CLEYFPSQMLRTGNNFQFSYEFENVPFHSSYAHSQSLDRLMNPLIDQYLY | |
| | YLSKTINGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGSLIFG | |
| | KQGTGHDNVDADKVMITNEEEIKTTNPVATESYGTVATNKQSAYAQAQTG | |
| | ALQSQGILPGMVWQDRDVYLQGPIWAKIPHTDGNFHPSPLMGGFGMKHP | |
| | PPQILIKNTPVPADPPTAFNKDKLNSFITQYSTGQVSVEIEWELQKENSKRW | |
| | NPEIQYTSNYYKSNNVEFAVNTEGVYSEPRPIGTRYLTRNL | |
| V27 (amino | MAADGYLPDWLEDNLSEGIREWWALKPGAPQPKANQQHQDNARGLVLP | 322 |
| acid) | GYKYLGPGNGLDKGEPVNAADAAALEHDKAYDQQLKAGDNPYLKYNHAD | |
| | AEFQERLKEDTSFGGNLGRAVFQAKKRLLEPLGLVEEAAKTAPGKKRPVE | |
| | QSPQEPDSSAGIGKSGAQPAKKRLNFGQTGDTESVPDPQPIGEPPAAPSG | |
| | VGSLTMASGGGAPVADNNEGADGVGSSSGNWHCDSQWLGDRVITTSTR | |
| | TWALPTYNNHLYKQISNSTSGGSSNDNAYFGYSTPWGYFDFNRFHCHFS | |
| | PRDWQRLINNNWGFRPKRLNFKLFNIQVKEVTDNNGVKTIANNLTSTVQVF | |
| | TDSDYQLPYVLGSAHEGCLPPFPADVFMIPQYGYLTLNDGSQAVGRSSFY | |
| | CLEYFPSQMLRTGNNFQFSYEFENVPFHSSYAHSQSLDRLMNPLIDQYLY | |
| | YLSKTINGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGSLIFG | |
| | KQGTGHDNVDADKVMITNEEEIKTTNPVATEAYGTVATNKQSAYAQAQTG | |
| | ALQNQGILPGMVWQDRDVYLQGPIWAKIPHTDGNFHPSPLMGGFGMKHP | |
| | PPQILIKNTPVPADPPTAFNKDKLNSFITQYSTGQVSVEIEWELQKENSKRW | |
| | NPEIQYTSNYYKSNNVEFAVNTEGVYSEPRPIGTRYLTRNL | |
| V28 (amino | MAADGYLPDWLEDNLSEGIREWWALKPGAPQPKANQQHQDNARGLVLP | 323 |
| acid) | GYKYLGPGNGLDKGEPVNAADAAALEHDKAYDQQLKAGDNPYLKYNHAD | |
| | AEFQERLKEDTSFGGNLGRAVFQAKKRLLEPLGLVEEAAKTAPGKKRPVE | |
| | QSPQEPDSSAGIGKSGAQPAKKRLNFGQTGDTESVPDPQPIGEPPAAPSG | |
| | VGSLTMASGGGAPVADNNEGADGVGSSSGNWHCDSQWLGDRVITTSTR | |
| | TWALPTYNNHLYKQISNSTSGGSSNDNAYFGYSTPWGYFDFNRFHCHFS | |
| | PRDWQRLINNNWGFRPKRLNFKLFNIQVKEVTDNNGVKTIANNLTSTVQVF | |
| | TDSDYQLPYVLGSAHEGCLPPFPADVFMIPQYGYLTLNDGSQAVGRSSFY | |
| | CLEYFPSQMLRTGNNFQFSYEFENVPFHSSYAHSQSLDRLMNPLIDQYLY | |
| | YLSKTINGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGSLIFG | |
| | KQGTGHDNVDADKVMITNEEEIKTTNPVATEAYGQVATNKQSAYAQAQTG | |
| | ALQSQGILPGMVWQDRDVYLQGPIWAKIPHTDGNFHPSPLMGGFGMKHP | |
| | PPQILIKNTPVPADPPTAFNKDKLNSFITQYSTGQVSVEIEWELQKENSKRW | |
| | NPEIQYTSNYYKSNNVEFAVNTEGVYSEPRPIGTRYLTRNL | |
| V29 (amino | MAADGYLPDWLEDNLSEGIREWWALKPGAPQPKANQQHQDNARGLVLP | 324 |
| acid) | GYKYLGPGNGLDKGEPVNAADAAALEHDKAYDQQLKAGDNPYLKYNHAD | |
| | AEFQERLKEDTSFGGNLGRAVFQAKKRLLEPLGLVEEAAKTAPGKKRPVE | |
| | QSPQEPDSSAGIGKSGAQPAKKRLNFGQTGDTESVPDPQPIGEPPAAPSG | |
| | VGSLTMASGGGAPVADNNEGADGVGSSSGNWHCDSQWLGDRVITTSTR | |
| | TWALPTYNNHLYKQISNSTSGGSSNDNAYFGYSTPWGYFDFNRFHCHFS | |
| | PRDWQRLINNNWGFRPKRLNFKLFNIQVKEVTDNNGVKTIANNLTSTVQVF | |
| | TDSDYQLPYVLGSAHEGCLPPFPADVFMIPQYGYLTLNDGSQAVGRSSFY | |
| | CLEYFPSQMLRTGNNFQFSYEFENVPFHSSYAHSQSLDRLMNPLIDQYLY | |
| | YLSKTINGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGSLIFG | |
| | KQGTGHDNVDADKVMITNEEEIKTTNPVATESYGTVATNKQSAYAQAQTG | |
| | ALQNQGVLPGMVWQDRDVYLQGPIWAKIPHTDGNFHPSPLMGGFGMKH | |
| | PPPQILIKNTPVPADPPTAFNKDKLNSFITQYSTGQVSVEIEWELQKENSKR | |
| | WNPEIQYTSNYYKSNNVEFAVNTEGVYSEPRPIGTRYLTRNL | |
| V30 (amino | MAADGYLPDWLEDNLSEGIREWWALKPGAPQPKANQQHQDNARGLVLP | 325 |
| acid) | GYKYLGPGNGLDKGEPVNAADAAALEHDKAYDQQLKAGDNPYLKYNHAD | |
| | AEFQERLKEDTSFGGNLGRAVFQAKKRLLEPLGLVEEAAKTAPGKKRPVE | |
| | QSPQEPDSSAGIGKSGAQPAKKRLNFGQTGDTESVPDPQPIGEPPAAPSG | |
| | VGSLTMASGGGAPVADNNEGADGVGSSSGNWHCDSQWLGDRVITTSTR | |
| | TWALPTYNNHLYKQISNSTSGGSSNDNAYFGYSTPWGYFDFNRFHCHFS | |
| | PRDWQRLINNNWGFRPKRLNFKLFNIQVKEVTDNNGVKTIANNLTSTVQVF | |
| | TDSDYQLPYVLGSAHEGCLPPFPADVFMIPQYGYLTLNDGSQAVGRSSFY | |
| | CLEYFPSQMLRTGNNFQFSYEFENVPFHSSYAHSQSLDRLMNPLIDQYLY | |
| | YLSKTINGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGSLIFG | |
| | KQGTGRDNVDADKVMITNEEEIKTTNPVATEAYGTVATNKQSAQAQAQTG | |
| | ALQSQGVLPGMVWQDRDVYLQGPIWAKIPHTDGNFHPSPLMGGFGMKHP | |
| | PPQILIKNTPVPADPPTAFNKDKLNSFITQYSTGQVSVEIEWELQKENSKRW | |
| | NPEIQYTSNYYKSNNVEFAVNTEGVYSEPRPIGTRYLTRNL | |
| V31 (amino | MAADGYLPDWLEDNLSEGIREWWALKPGAPQPKANQQHQDNARGLVLP | 326 |
| acid) | GYKYLGPGNGLDKGEPVNAADAAALEHDKAYDQQLKAGDNPYLKYNHAD | |
| | AEFQERLKEDTSFGGNLGRAVFQAKKRLLEPLGLVEEAAKTAPGKKRPVE | |
| | QSPQEPDSSAGIGKSGAQPAKKRLNFGQTGDTESVPDPQPIGEPPAAPSG | |
| | VGSLTMASGGGAPVADNNEGADGVGSSSGNWHCDSQWLGDRVITTSTR | |
| | TWALPTYNNHLYKQISNSTSGGSSNDNAYFGYSTPWGYFDFNRFHCHFS | |
| | PRDWQRLINNNWGFRPKRLNFKLFNIQVKEVTDNNGVKTIANNLTSTVQVF | |
| | TDSDYQLPYVLGSAHEGCLPPFPADVFMIPQYGYLTLNDGSQAVGRSSFY | |
| | CLEYFPSQMLRTGNNFQFSYEFENVPFHSSYAHSQSLDRLMNPLIDQYLY | |
| | YLSKTINGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGSLIFG | |
| | KQGTGHDNVDADKVMITNEEEIKTTNPVATEAYGTVATNHQSAYAQAQTG | |
| | ALQNQGVLPGMVWQDRDVYLQGPIWAKIPHTDGNFHPSPLMGGFGMKH | |
| | PPPQILIKNTPVPADPPTAFNKDKLNSFITQYSTGQVSVEIEWELQKENSKR | |
| | WNPEIQYTSNYYKSNNVEFAVNTEGVYSEPRPIGTRYLTRNL | |
| V32 (amino | MAADGYLPDWLEDNLSEGIREWWALKPGAPQPKANQQHQDNARGLVLP | 327 |
| acid) | GYKYLGPGNGLDKGEPVNAADAAALEHDKAYDQQLKAGDNPYLKYNHAD | |
| | AEFQERLKEDTSFGGNLGRAVFQAKKRLLEPLGLVEEAAKTAPGKKRPVE | |
| | QSPQEPDSSAGIGKSGAQPAKKRLNFGQTGDTESVPDPQPIGEPPAAPSG | |
| | VGSLTMASGGGAPVADNNEGADGVGSSSGNWHCDSQWLGDRVITTSTR | |
| | TWALPTYNNHLYKQISNSTSGGSSNDNAYFGYSTPWGYFDFNRFHCHFS | |
| | PRDWQRLINNNWGFRPKRLNFKLFNIQVKEVTDNNGVKTIANNLTSTVQVF | |
| | TDSDYQLPYVLGSAHEGCLPPFPADVFMIPQYGYLTLNDGSQAVGRSSFY | |
| | CLEYFPSQMLRTGNNFQFSYEFENVPFHSSYAHSQSLDRLMNPLIDQYLY | |
| | YLSKTINGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGSLIFG | |
| | KQGTGHDNVDADKVMITNEEEIKTTNPVATEAYGQVATNKQSAYAQAQTG | |
| | AVQSQGVLPGMVWQDRDVYLQGPIWAKIPHTDGNFHPSPLMGGFGMKH | |
| | PPPQILIKNTPVPADPPTAFNKDKLNSFITQYSTGQVSVEIEWELQKENSKR | |
| | WNPEIQYTSNYYKSNNVEFAVNTEGVYSEPRPIGTRYLTRNL | |
| V33 (amino | MAADGYLPDWLEDNLSEGIREWWALKPGAPQPKANQQHQDNARGLVLP | 328 |
| acid) | GYKYLGPGNGLDKGEPVNAADAAALEHDKAYDQQLKAGDNPYLKYNHAD | |
| | AEFQERLKEDTSFGGNLGRAVFQAKKRLLEPLGLVEEAAKTAPGKKRPVE | |
| | QSPQEPDSSAGIGKSGAQPAKKRLNFGQTGDTESVPDPQPIGEPPAAPSG | |
| | VGSLTMASGGGAPVADNNEGADGVGSSSGNWHCDSQWLGDRVITTSTR | |
| | TWALPTYNNHLYKQISNSTSGGSSNDNAYFGYSTPWGYFDFNRFHCHFS | |
| | PRDWQRLINNNWGFRPKRLNFKLFNIQVKEVTDNNGVKTIANNLTSTVQVF | |
| | TDSDYQLPYVLGSAHEGCLPPFPADVFMIPQYGYLTLNDGSQAVGRSSFY | |
| | CLEYFPSQMLRTGNNFQFSYEFENVPFHSSYAHSQSLDRLMNPLIDQYLY | |
| | YLSKTINGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGSLIFG | |
| | KQGTGRDNVDADKVMITNEEEIKTTNPVATEAYGTVATNKQSAYAQAQTG | |
| | ALQNQGVLPGMVWQDRDVYLQGPIWAKIPHTDGNFHPSPLMGGFGMKH | |
| | PPPQILIKNTPVPADPPTAFNKDKLNSFITQYSTGQVSVEIEWELQKENSKR | |
| | WNPEIQYTSNYYKSNNVEFAVNTEGVYSEPRPIGTRYLTRNL | |
| V34 (amino | MAADGYLPDWLEDNLSEGIREWWALKPGAPQPKANQQHQDNARGLVLP | 329 |
| acid) | GYKYLGPGNGLDKGEPVNAADAAALEHDKAYDQQLKAGDNPYLKYNHAD | |
| | AEFQERLKEDTSFGGNLGRAVFQAKKRLLEPLGLVEEAAKTAPGKKRPVE | |
| | QSPQEPDSSAGIGKSGAQPAKKRLNFGQTGDTESVPDPQPIGEPPAAPSG | |
| | VGSLTMASGGGAPVADNNEGADGVGSSSGNWHCDSQWLGDRVITTSTR | |
| | TWALPTYNNHLYKQISNSTSGGSSNDNAYFGYSTPWGYFDFNRFHCHFS | |
| | PRDWQRLINNNWGFRPKRLNFKLFNIQVKEVTDNNGVKTIANNLTSTVQVF | |
| | TDSDYQLPYVLGSAHEGCLPPFPADVFMIPQYGYLTLNDGSQAVGRSSFY | |
| | CLEYFPSQMLRTGNNFQFSYEFENVPFHSSYAHSQSLDRLMNPLIDQYLY | |
| | YLSKTINGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGSLIFG | |
| | KQGTGHDNVDADKVMITNEEEIKTTNPVATEAYGTVATNKQSAQAQAQTG | |
| | WLQSQGVLPGMVWQDRDVYLQGPIWAKIPHTDGNFHPSPLMGGFGMKH | |
| | PPPQILIKNTPVPADPPTAFNKDKLNSFITQYSTGQVSVEIEWELQKENSKR | |
| | WNPEIQYTSNYYKSNNVEFAVNTEGVYSEPRPIGTRYLTRNL | |
| V35 (amino | MAADGYLPDWLEDNLSEGIREWWALKPGAPQPKANQQHQDNARGLVLP | 330 |
| acid) | GYKYLGPGNGLDKGEPVNAADAAALEHDKAYDQQLKAGDNPYLKYNHAD | |
| | AEFQERLKEDTSFGGNLGRAVFQAKKRLLEPLGLVEEAAKTAPGKKRPVE | |
| | QSPQEPDSSAGIGKSGAQPAKKRLNFGQTGDTESVPDPQPIGEPPAAPSG | |
| | VGSLTMASGGGAPVADNNEGADGVGSSSGNWHCDSQWLGDRVITTSTR | |
| | TWALPTYNNHLYKQISNSTSGGSSNDNAYFGYSTPWGYFDFNRFHCHFS | |
| | PRDWQRLINNNWGFRPKRLNFKLFNIQVKEVTDNNGVKTIANNLTSTVQVF | |
| | TDSDYQLPYVLGSAHEGCLPPFPADVFMIPQYGYLTLNDGSQAVGRSSFY | |
| | CLEYFPSQMLRTGNNFQFSYEFENVPFHSSYAHSQSLDRLMNPLIDQYLY | |
| | YLSKTINGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGSLIFG | |
| | KQGTGRDNVDADKVMITNEEEIKTTNPVATEAYGTVATNHQSAYAQAQTG | |
| | ALQSQGVLPGMVWQDRDVYLQGPIWAKIPHTDGNFHPSPLMGGFGMKHP | |
| | PPQILIKNTPVPADPPTAFNKDKLNSFITQYSTGQVSVEIEWELQKENSKRW | |
| | NPEIQYTSNYYKSNNVEFAVNTEGVYSEPRPIGTRYLTRNL | |
| V36 (amino | MAADGYLPDWLEDNLSEGIREWWALKPGAPQPKANQQHQDNARGLVLP | 331 |
| acid) | GYKYLGPGNGLDKGEPVNAADAAALEHDKAYDQQLKAGDNPYLKYNHAD | |
| | AEFQERLKEDTSFGGNLGRAVFQAKKRLLEPLGLVEEAAKTAPGKKRPVE | |
| | QSPQEPDSSAGIGKSGAQPAKKRLNFGQTGDTESVPDPQPIGEPPAAPSG | |
| | VGSLTMASGGGAPVADNNEGADGVGSSSGNWHCDSQWLGDRVITTSTR | |
| | TWALPTYNNHLYKQISNSTSGGSSNDNAYFGYSTPWGYFDFNRFHCHFS | |
| | PRDWQRLINNNWGFRPKRLNFKLFNIQVKEVTDNNGVKTIANNLTSTVQVF | |
| | TDSDYQLPYVLGSAHEGCLPPFPADVFMIPQYGYLTLNDGSQAVGRSSFY | |
| | CLEYFPSQMLRTGNNFQFSYEFENVPFHSSYAHSQSLDRLMNPLIDQYLY | |
| | YLSKTINGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGSLIFG | |
| | KQGTGHDNVDADKVMITNEEEIKTTNPVATEAYGTVATNHQSAQAQAQTG | |
| | ALQSQGVLPGMVWQDRDVYLQGPIWAKIPHTDGNFHPSPLMGGFGMKHP | |
| | PPQILIKNTPVPADPPTAFNKDKLNSFITQYSTGQVSVEIEWELQKENSKRW | |
| | NPEIQYTSNYYKSNNVEFAVNTEGVYSEPRPIGTRYLTRNL | |
| V37 (amino | MAADGYLPDWLEDNLSEGIREWWALKPGAPQPKANQQHQDNARGLVLP | 332 |
| acid) | GYKYLGPGNGLDKGEPVNAADAAALEHDKAYDQQLKAGDNPYLKYNHAD | |
| | AEFQERLKEDTSFGGNLGRAVFQAKKRLLEPLGLVEEAAKTAPGKKRPVE | |
| | QSPQEPDSSAGIGKSGAQPAKKRLNFGQTGDTESVPDPQPIGEPPAAPSG | |
| | VGSLTMASGGGAPVADNNEGADGVGSSSGNWHCDSQWLGDRVITTSTR | |
| | TWALPTYNNHLYKQISNSTSGGSSNDNAYFGYSTPWGYFDFNRFHCHFS | |
| | PRDWQRLINNNWGFRPKRLNFKLFNIQVKEVTDNNGVKTIANNLTSTVQVF | |
| | TDSDYQLPYVLGSAHEGCLPPFPADVFMIPQYGYLTLNDGSQAVGRSSFY | |
| | CLEYFPSQMLRTGNNFQFSYEFENVPFHSSYAHSQSLDRLMNPLIDQYLY | |
| | YLSKTINGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGSLIFG | |
| | KQGTGHDNVDADKVMITNEEEIKTTNPVATESYGTVATNKQSAQAQAQTG | |
| | ALQSQGVLPGMVWQDRDVYLQGPIWAKIPHTDGNFHPSPLMGGFGMKHP | |
| | PPQILIKNTPVPADPPTAFNKDKLNSFITQYSTGQVSVEIEWELQKENSKRW | |
| | NPEIQYTSNYYKSNNVEFAVNTEGVYSEPRPIGTRYLTRNL | |
| V38 (amino | MAADGYLPDWLEDNLSEGIREWWALKPGAPQPKANQQHQDNARGLVLP | 333 |
| acid) | GYKYLGPGNGLDKGEPVNAADAAALEHDKAYDQQLKAGDNPYLKYNHAD | |
| | AEFQERLKEDTSFGGNLGRAVFQAKKRLLEPLGLVEEAAKTAPGKKRPVE | |
| | QSPQEPDSSAGIGKSGAQPAKKRLNFGQTGDTESVPDPQPIGEPPAAPSG | |
| | VGSLTMASGGGAPVADNNEGADGVGSSSGNWHCDSQWLGDRVITTSTR | |
| | TWALPTYNNHLYKQISNSTSGGSSNDNAYFGYSTPWGYFDFNRFHCHFS | |
| | PRDWQRLINNNWGFRPKRLNFKLFNIQVKEVTDNNGVKTIANNLTSTVQVF | |
| | TDSDYQLPYVLGSAHEGCLPPFPADVFMIPQYGYLTLNDGSQAVGRSSFY | |
| | CLEYFPSQMLRTGNNFQFSYEFENVPFHSSYAHSQSLDRLMNPLIDQYLY | |
| | YLSKTINGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGSLIFG | |
| | KQGTGRDNVDADKVMITNEEEIKTTNPVATESYGTVATNHQSAYAQAQTG | |
| | ALQSQGVLPGMVWQDRDVYLQGPIWAKIPHTDGNFHPSPLMGGFGMKHP | |
| | PPQILIKNTPVPADPPTAFNKDKLNSFITQYSTGQVSVEIEWELQKENSKRW | |
| | NPEIQYTSNYYKSNNVEFAVNTEGVYSEPRPIGTRYLTRNL | |
| V39 (amino | MAADGYLPDWLEDNLSEGIREWWALKPGAPQPKANQQHQDNARGLVLP | 334 |
| acid) | GYKYLGPGNGLDKGEPVNAADAAALEHDKAYDQQLKAGDNPYLKYNHAD | |
| | AEFQERLKEDTSFGGNLGRAVFQAKKRLLEPLGLVEEAAKTAPGKKRPVE | |
| | QSPQEPDSSAGIGKSGAQPAKKRLNFGQTGDTESVPDPQPIGEPPAAPSG | |
| | VGSLTMASGGGAPVADNNEGADGVGSSSGNWHCDSQWLGDRVITTSTR | |
| | TWALPTYNNHLYKQISNSTSGGSSNDNAYFGYSTPWGYFDFNRFHCHFS | |
| | PRDWQRLINNNWGFRPKRLNFKLFNIQVKEVTDNNGVKTIANNLTSTVQVF | |
| | TDSDYQLPYVLGSAHEGCLPPFPADVFMIPQYGYLTLNDGSQAVGRSSFY | |
| | CLEYFPSQMLRTGNNFQFSYEFENVPFHSSYAHSQSLDRLMNPLIDQYLY | |
| | YLSKTINGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGSLIFG | |
| | KQGTGRDNVDADKVMITNEEEIKTTNPVATESYGQVATNKQSAYAQAQTG | |
| | ALQSQGVLPGMVWQDRDVYLQGPIWAKIPHTDGNFHPSPLMGGFGMKHP | |
| | PPQILIKNTPVPADPPTAFNKDKLNSFITQYSTGQVSVEIEWELQKENSKRW | |
| | NPEIQYTSNYYKSNNVEFAVNTEGVYSEPRPIGTRYLTRNL | |
| V40 (amino | MAADGYLPDWLEDNLSEGIREWWALKPGAPQPKANQQHQDNARGLVLP | 335 |
| acid) | GYKYLGPGNGLDKGEPVNAADAAALEHDKAYDQQLKAGDNPYLKYNHAD | |
| | AEFQERLKEDTSFGGNLGRAVFQAKKRLLEPLGLVEEAAKTAPGKKRPVE | |
| | QSPQEPDSSAGIGKSGAQPAKKRLNFGQTGDTESVPDPQPIGEPPAAPSG | |
| | VGSLTMASGGGAPVADNNEGADGVGSSSGNWHCDSQWLGDRVITTSTR | |
| | TWALPTYNNHLYKQISNSTSGGSSNDNAYFGYSTPWGYFDFNRFHCHFS | |
| | PRDWQRLINNNWGFRPKRLNFKLFNIQVKEVTDNNGVKTIANNLTSTVQVF | |
| | TDSDYQLPYVLGSAHEGCLPPFPADVFMIPQYGYLTLNDGSQAVGRSSFY | |
| | CLEYFPSQMLRTGNNFQFSYEFENVPFHSSYAHSQSLDRLMNPLIDQYLY | |
| | YLSKTINGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGSLIFG | |
| | KQGTGHDNVDADKVMITNEEEIKTTNPVATEAYGTVATNHQSAYAQAQTG | |
| | AVQSQGILPGMVWQDRDVYLQGPIWAKIPHTDGNFHPSPLMGGFGMKHP | |
| | PPQILIKNTPVPADPPTAFNKDKLNSFITQYSTGQVSVEIEWELQKENSKRW | |
| | NPEIQYTSNYYKSNNVEFAVNTEGVYSEPRPIGTRYLTRNL | |
| V41 (amino | MAADGYLPDWLEDNLSEGIREWWALKPGAPQPKANQQHQDNARGLVLP | 336 |
| acid) | GYKYLGPGNGLDKGEPVNAADAAALEHDKAYDQQLKAGDNPYLKYNHAD | |
| | AEFQERLKEDTSFGGNLGRAVFQAKKRLLEPLGLVEEAAKTAPGKKRPVE | |
| | QSPQEPDSSAGIGKSGAQPAKKRLNFGQTGDTESVPDPQPIGEPPAAPSG | |
| | VGSLTMASGGGAPVADNNEGADGVGSSSGNWHCDSQWLGDRVITTSTR | |
| | TWALPTYNNHLYKQISNSTSGGSSNDNAYFGYSTPWGYFDFNRFHCHFS | |
| | PRDWQRLINNNWGFRPKRLNFKLFNIQVKEVTDNNGVKTIANNLTSTVQVF | |
| | TDSDYQLPYVLGSAHEGCLPPFPADVFMIPQYGYLTLNDGSQAVGRSSFY | |
| | CLEYFPSQMLRTGNNFQFSYEFENVPFHSSYAHSQSLDRLMNPLIDQYLY | |
| | YLSKTINGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGSLIFG | |
| | KQGTGHDNVDADKVMITNEEEIKTTNPVATEAYGQVATNKQSAYAQAQTG | |
| | AVQNQGVLPGMVWQDRDVYLQGPIWAKIPHTDGNFHPSPLMGGFGMKH | |
| | PPPQILIKNTPVPADPPTAFNKDKLNSFITQYSTGQVSVEIEWELQKENSKR | |
| | WNPEIQYTSNYYKSNNVEFAVNTEGVYSEPRPIGTRYLTRNL | |
| V42 (amino | MAADGYLPDWLEDNLSEGIREWWALKPGAPQPKANQQHQDNARGLVLP | 337 |
| acid) | GYKYLGPGNGLDKGEPVNAADAAALEHDKAYDQQLKAGDNPYLKYNHAD | |
| | AEFQERLKEDTSFGGNLGRAVFQAKKRLLEPLGLVEEAAKTAPGKKRPVE | |
| | QSPQEPDSSAGIGKSGAQPAKKRLNFGQTGDTESVPDPQPIGEPPAAPSG | |
| | VGSLTMASGGGAPVADNNEGADGVGSSSGNWHCDSQWLGDRVITTSTR | |
| | TWALPTYNNHLYKQISNSTSGGSSNDNAYFGYSTPWGYFDFNRFHCHFS | |
| | PRDWQRLINNNWGFRPKRLNFKLFNIQVKEVTDNNGVKTIANNLTSTVQVF | |
| | TDSDYQLPYVLGSAHEGCLPPFPADVFMIPQYGYLTLNDGSQAVGRSSFY | |
| | CLEYFPSQMLRTGNNFQFSYEFENVPFHSSYAHSQSLDRLMNPLIDQYLY | |
| | YLSKTINGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGSLIFG | |
| | KQGTGHDNVDADKVMITNEEEIKTTNPVATESYGQVATNKQSAYAQAQTG | |
| | ALQNQGVLPGMVWQDRDVYLQGPIWAKIPHTDGNFHPSPLMGGFGMKH | |
| | PPPQILIKNTPVPADPPTAFNKDKLNSFITQYSTGQVSVEIEWELQKENSKR | |
| | WNPEIQYTSNYYKSNNVEFAVNTEGVYSEPRPIGTRYLTRNL | |
| V43 (amino | MAADGYLPDWLEDNLSEGIREWWALKPGAPQPKANQQHQDNARGLVLP | 338 |
| acid) | GYKYLGPGNGLDKGEPVNAADAAALEHDKAYDQQLKAGDNPYLKYNHAD | |
| | AEFQERLKEDTSFGGNLGRAVFQAKKRLLEPLGLVEEAAKTAPGKKRPVE | |
| | QSPQEPDSSAGIGKSGAQPAKKRLNFGQTGDTESVPDPQPIGEPPAAPSG | |
| | VGSLTMASGGGAPVADNNEGADGVGSSSGNWHCDSQWLGDRVITTSTR | |
| | TWALPTYNNHLYKQISNSTSGGSSNDNAYFGYSTPWGYFDFNRFHCHFS | |
| | PRDWQRLINNNWGFRPKRLNFKLFNIQVKEVTDNNGVKTIANNLTSTVQVF | |
| | TDSDYQLPYVLGSAHEGCLPPFPADVFMIPQYGYLTLNDGSQAVGRSSFY | |
| | CLEYFPSQMLRTGNNFQFSYEFENVPFHSSYAHSQSLDRLMNPLIDQYLY | |
| | YLSKTINGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGSLIFG | |
| | KQGTGRDNVDADKVMITNEEEIKTTNPVATEAYGTVATNKQSAYAQAQTG | |
| | AVQSQGILPGMVWQDRDVYLQGPIWAKIPHTDGNFHPSPLMGGFGMKHP | |
| | PPQILIKNTPVPADPPTAFNKDKLNSFITQYSTGQVSVEIEWELQKENSKRW | |
| | NPEIQYTSNYYKSNNVEFAVNTEGVYSEPRPIGTRYLTRNL | |
| V44 (amino | MAADGYLPDWLEDNLSEGIREWWALKPGAPQPKANQQHQDNARGLVLP | 339 |
| acid) | GYKYLGPGNGLDKGEPVNAADAAALEHDKAYDQQLKAGDNPYLKYNHAD | |
| | AEFQERLKEDTSFGGNLGRAVFQAKKRLLEPLGLVEEAAKTAPGKKRPVE | |
| | QSPQEPDSSAGIGKSGAQPAKKRLNFGQTGDTESVPDPQPIGEPPAAPSG | |
| | VGSLTMASGGGAPVADNNEGADGVGSSSGNWHCDSQWLGDRVITTSTR | |
| | TWALPTYNNHLYKQISNSTSGGSSNDNAYFGYSTPWGYFDFNRFHCHFS | |
| | PRDWQRLINNNWGFRPKRLNFKLFNIQVKEVTDNNGVKTIANNLTSTVQVF | |
| | TDSDYQLPYVLGSAHEGCLPPFPADVFMIPQYGYLTLNDGSQAVGRSSFY | |
| | CLEYFPSQMLRTGNNFQFSYEFENVPFHSSYAHSQSLDRLMNPLIDQYLY | |
| | YLSKTINGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGSLIFG | |
| | KQGTGHDNVDADKVMITNEEEIKTTNPVATESYGQVATNKQSAYAQAQTG | |
| | ALQSQGILPGMVWQDRDVYLQGPIWAKIPHTDGNFHPSPLMGGFGMKHP | |
| | PPQILIKNTPVPADPPTAFNKDKLNSFITQYSTGQVSVEIEWELQKENSKRW | |
| | NPEIQYTSNYYKSNNVEFAVNTEGVYSEPRPIGTRYLTRNL | |
| V45 (amino | MAADGYLPDWLEDNLSEGIREWWALKPGAPQPKANQQHQDNARGLVLP | 340 |
| acid) | GYKYLGPGNGLDKGEPVNAADAAALEHDKAYDQQLKAGDNPYLKYNHAD | |
| | AEFQERLKEDTSFGGNLGRAVFQAKKRLLEPLGLVEEAAKTAPGKKRPVE | |
| | QSPQEPDSSAGIGKSGAQPAKKRLNFGQTGDTESVPDPQPIGEPPAAPSG | |
| | VGSLTMASGGGAPVADNNEGADGVGSSSGNWHCDSQWLGDRVITTSTR | |
| | TWALPTYNNHLYKQISNSTSGGSSNDNAYFGYSTPWGYFDFNRFHCHFS | |
| | PRDWQRLINNNWGFRPKRLNFKLFNIQVKEVTDNNGVKTIANNLTSTVQVF | |
| | TDSDYQLPYVLGSAHEGCLPPFPADVFMIPQYGYLTLNDGSQAVGRSSFY | |
| | CLEYFPSQMLRTGNNFQFSYEFENVPFHSSYAHSQSLDRLMNPLIDQYLY | |
| | YLSKTINGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGSLIFG | |
| | KQGTGRDNVDADKVMITNEEEIKTTNPVATEAYGTVATNKQSAYAQAQTG | |
| | AVQNQGVLPGMVWQDRDVYLQGPIWAKIPHTDGNFHPSPLMGGFGMKH | |
| | PPPQILIKNTPVPADPPTAFNKDKLNSFITQYSTGQVSVEIEWELQKENSKR | |
| | WNPEIQYTSNYYKSNNVEFAVNTEGVYSEPRPIGTRYLTRNL | |
| V46 (amino | MAADGYLPDWLEDNLSEGIREWWALKPGAPQPKANQQHQDNARGLVLP | 341 |
| acid) | GYKYLGPGNGLDKGEPVNAADAAALEHDKAYDQQLKAGDNPYLKYNHAD | |
| | AEFQERLKEDTSFGGNLGRAVFQAKKRLLEPLGLVEEAAKTAPGKKRPVE | |
| | QSPQEPDSSAGIGKSGAQPAKKRLNFGQTGDTESVPDPQPIGEPPAAPSG | |
| | VGSLTMASGGGAPVADNNEGADGVGSSSGNWHCDSQWLGDRVITTSTR | |
| | TWALPTYNNHLYKQISNSTSGGSSNDNAYFGYSTPWGYFDFNRFHCHFS | |
| | PRDWQRLINNNWGFRPKRLNFKLFNIQVKEVTDNNGVKTIANNLTSTVQVF | |
| | TDSDYQLPYVLGSAHEGCLPPFPADVFMIPQYGYLTLNDGSQAVGRSSFY | |
| | CLEYFPSQMLRTGNNFQFSYEFENVPFHSSYAHSQSLDRLMNPLIDQYLY | |
| | YLSKTINGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGSLIFG | |
| | KQGTGHDNVDADKVMITNEEEIKTTNPVATEAYGTVATNKQSAQAQAQTG | |
| | WVQSQGVLPGMVWQDRDVYLQGPIWAKIPHTDGNFHPSPLMGGFGMKH | |
| | PPPQILIKNTPVPADPPTAFNKDKLNSFITQYSTGQVSVEIEWELQKENSKR | |
| | WNPEIQYTSNYYKSNNVEFAVNTEGVYSEPRPIGTRYLTRNL | |
| V47 (amino | MAADGYLPDWLEDNLSEGIREWWALKPGAPQPKANQQHQDNARGLVLP | 342 |
| acid) | GYKYLGPGNGLDKGEPVNAADAAALEHDKAYDQQLKAGDNPYLKYNHAD | |
| | AEFQERLKEDTSFGGNLGRAVFQAKKRLLEPLGLVEEAAKTAPGKKRPVE | |
| | QSPQEPDSSAGIGKSGAQPAKKRLNFGQTGDTESVPDPQPIGEPPAAPSG | |
| | VGSLTMASGGGAPVADNNEGADGVGSSSGNWHCDSQWLGDRVITTSTR | |
| | TWALPTYNNHLYKQISNSTSGGSSNDNAYFGYSTPWGYFDFNRFHCHFS | |
| | PRDWQRLINNNWGFRPKRLNFKLFNIQVKEVTDNNGVKTIANNLTSTVQVF | |
| | TDSDYQLPYVLGSAHEGCLPPFPADVFMIPQYGYLTLNDGSQAVGRSSFY | |
| | CLEYFPSQMLRTGNNFQFSYEFENVPFHSSYAHSQSLDRLMNPLIDQYLY | |
| | YLSKTINGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGSLIFG | |
| | KQGTGRDNVDADKVMITNEEEIKTTNPVATEAYGQVATNHQSAYAQAQTG | |
| | ALQSQGVLPGMVWQDRDVYLQGPIWAKIPHTDGNFHPSPLMGGFGMKHP | |
| | PPQILIKNTPVPADPPTAFNKDKLNSFITQYSTGQVSVEIEWELQKENSKRW | |
| | NPEIQYTSNYYKSNNVEFAVNTEGVYSEPRPIGTRYLTRNL | |
| V48 (amino | MAADGYLPDWLEDNLSEGIREWWALKPGAPQPKANQQHQDNARGLVLP | 343 |
| acid) | GYKYLGPGNGLDKGEPVNAADAAALEHDKAYDQQLKAGDNPYLKYNHAD | |
| | AEFQERLKEDTSFGGNLGRAVFQAKKRLLEPLGLVEEAAKTAPGKKRPVE | |
| | QSPQEPDSSAGIGKSGAQPAKKRLNFGQTGDTESVPDPQPIGEPPAAPSG | |
| | VGSLTMASGGGAPVADNNEGADGVGSSSGNWHCDSQWLGDRVITTSTR | |
| | TWALPTYNNHLYKQISNSTSGGSSNDNAYFGYSTPWGYFDFNRFHCHFS | |
| | PRDWQRLINNNWGFRPKRLNFKLFNIQVKEVTDNNGVKTIANNLTSTVQVF | |
| | TDSDYQLPYVLGSAHEGCLPPFPADVFMIPQYGYLTLNDGSQAVGRSSFY | |
| | CLEYFPSQMLRTGNNFQFSYEFENVPFHSSYAHSQSLDRLMNPLIDQYLY | |
| | YLSKTINGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGSLIFG | |
| | KQGTGHDNVDADKVMITNEEEIKTTNPVATEAYGTVATNKQSAYAQAQTG | |
| | AVQNQGILPGMVWQDRDVYLQGPIWAKIPHTDGNFHPSPLMGGFGMKHP | |
| | PPQILIKNTPVPADPPTAFNKDKLNSFITQYSTGQVSVEIEWELQKENSKRW | |
| | NPEIQYTSNYYKSNNVEFAVNTEGVYSEPRPIGTRYLTRNL | |
| V49 (amino | MAADGYLPDWLEDNLSEGIREWWALKPGAPQPKANQQHQDNARGLVLP | 344 |
| acid) | GYKYLGPGNGLDKGEPVNAADAAALEHDKAYDQQLKAGDNPYLKYNHAD | |
| | AEFQERLKEDTSFGGNLGRAVFQAKKRLLEPLGLVEEAAKTAPGKKRPVE | |
| | QSPQEPDSSAGIGKSGAQPAKKRLNFGQTGDTESVPDPQPIGEPPAAPSG | |
| | VGSLTMASGGGAPVADNNEGADGVGSSSGNWHCDSQWLGDRVITTSTR | |
| | TWALPTYNNHLYKQISNSTSGGSSNDNAYFGYSTPWGYFDFNRFHCHFS | |
| | PRDWQRLINNNWGFRPKRLNFKLFNIQVKEVTDNNGVKTIANNLTSTVQVF | |
| | TDSDYQLPYVLGSAHEGCLPPFPADVFMIPQYGYLTLNDGSQAVGRSSFY | |
| | CLEYFPSQMLRTGNNFQFSYEFENVPFHSSYAHSQSLDRLMNPLIDQYLY | |
| | YLSKTINGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGSLIFG | |
| | KQGTGRDNVDADKVMITNEEEIKTTNPVATEAYGQVATNKQSAYAQAQTG | |
| | ALQSQGILPGMVWQDRDVYLQGPIWAKIPHTDGNFHPSPLMGGFGMKHP | |
| | PPQILIKNTPVPADPPTAFNKDKLNSFITQYSTGQVSVEIEWELQKENSKRW | |
| | NPEIQYTSNYYKSNNVEFAVNTEGVYSEPRPIGTRYLTRNL | |
| V50 (amino | MAADGYLPDWLEDNLSEGIREWWALKPGAPQPKANQQHQDNARGLVLP | 345 |
| acid) | GYKYLGPGNGLDKGEPVNAADAAALEHDKAYDQQLKAGDNPYLKYNHAD | |
| | AEFQERLKEDTSFGGNLGRAVFQAKKRLLEPLGLVEEAAKTAPGKKRPVE | |
| | QSPQEPDSSAGIGKSGAQPAKKRLNFGQTGDTESVPDPQPIGEPPAAPSG | |
| | VGSLTMASGGGAPVADNNEGADGVGSSSGNWHCDSQWLGDRVITTSTR | |
| | TWALPTYNNHLYKQISNSTSGGSSNDNAYFGYSTPWGYFDFNRFHCHFS | |
| | PRDWQRLINNNWGFRPKRLNFKLFNIQVKEVTDNNGVKTIANNLTSTVQVF | |
| | TDSDYQLPYVLGSAHEGCLPPFPADVFMIPQYGYLTLNDGSQAVGRSSFY | |
| | CLEYFPSQMLRTGNNFQFSYEFENVPFHSSYAHSQSLDRLMNPLIDQYLY | |
| | YLSKTINGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGSLIFG | |
| | KQGTGRDNVDADKVMITNEEEIKTTNPVATEAYGTVATNKQSAYAQAQTG | |
| | WVQSQGVLPGMVWQDRDVYLQGPIWAKIPHTDGNFHPSPLMGGFGMKH | |
| | PPPQILIKNTPVPADPPTAFNKDKLNSFITQYSTGQVSVEIEWELQKENSKR | |
| | WNPEIQYTSNYYKSNNVEFAVNTEGVYSEPRPIGTRYLTRNL | |
| V51 (amino | MAADGYLPDWLEDNLSEGIREWWALKPGAPQPKANQQHQDNARGLVLP | 346 |
| acid) | GYKYLGPGNGLDKGEPVNAADAAALEHDKAYDQQLKAGDNPYLKYNHAD | |
| | AEFQERLKEDTSFGGNLGRAVFQAKKRLLEPLGLVEEAAKTAPGKKRPVE | |
| | QSPQEPDSSAGIGKSGAQPAKKRLNFGQTGDTESVPDPQPIGEPPAAPSG | |
| | VGSLTMASGGGAPVADNNEGADGVGSSSGNWHCDSQWLGDRVITTSTR | |
| | TWALPTYNNHLYKQISNSTSGGSSNDNAYFGYSTPWGYFDFNRFHCHFS | |
| | PRDWQRLINNNWGFRPKRLNFKLFNIQVKEVTDNNGVKTIANNLTSTVQVF | |
| | TDSDYQLPYVLGSAHEGCLPPFPADVFMIPQYGYLTLNDGSQAVGRSSFY | |
| | CLEYFPSQMLRTGNNFQFSYEFENVPFHSSYAHSQSLDRLMNPLIDQYLY | |
| | YLSKTINGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGSLIFG | |
| | KQGTGRDNVDADKVMITNEEEIKTTNPVATEAYGTVATNHQSAYAQAQTG | |
| | ALQSQGILPGMVWQDRDVYLQGPIWAKIPHTDGNFHPSPLMGGFGMKHP | |
| | PPQILIKNTPVPADPPTAFNKDKLNSFITQYSTGQVSVEIEWELQKENSKRW | |
| | NPEIQYTSNYYKSNNVEFAVNTEGVYSEPRPIGTRYLTRNL | |
| V52 (amino | MAADGYLPDWLEDNLSEGIREWWALKPGAPQPKANQQHQDNARGLVLP | 347 |
| acid) | GYKYLGPGNGLDKGEPVNAADAAALEHDKAYDQQLKAGDNPYLKYNHAD | |
| | AEFQERLKEDTSFGGNLGRAVFQAKKRLLEPLGLVEEAAKTAPGKKRPVE | |
| | QSPQEPDSSAGIGKSGAQPAKKRLNFGQTGDTESVPDPQPIGEPPAAPSG | |
| | VGSLTMASGGGAPVADNNEGADGVGSSSGNWHCDSQWLGDRVITTSTR | |
| | TWALPTYNNHLYKQISNSTSGGSSNDNAYFGYSTPWGYFDFNRFHCHFS | |
| | PRDWQRLINNNWGFRPKRLNFKLFNIQVKEVTDNNGVKTIANNLTSTVQVF | |
| | TDSDYQLPYVLGSAHEGCLPPFPADVFMIPQYGYLTLNDGSQAVGRSSFY | |
| | CLEYFPSQMLRTGNNFQFSYEFENVPFHSSYAHSQSLDRLMNPLIDQYLY | |
| | YLSKTINGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGSLIFG | |
| | KQGTGRDNVDADKVMITNEEEIKTTNPVATEAYGQVATNKQSAYAQAQTG | |
| | ALQNQGVLPGMVWQDRDVYLQGPIWAKIPHTDGNFHPSPLMGGFGMKH | |
| | PPPQILIKNTPVPADPPTAFNKDKLNSFITQYSTGQVSVEIEWELQKENSKR | |
| | WNPEIQYTSNYYKSNNVEFAVNTEGVYSEPRPIGTRYLTRNL | |
| V53 (amino | MAADGYLPDWLEDNLSEGIREWWALKPGAPQPKANQQHQDNARGLVLP | 348 |
| acid) | GYKYLGPGNGLDKGEPVNAADAAALEHDKAYDQQLKAGDNPYLKYNHAD | |
| | AEFQERLKEDTSFGGNLGRAVFQAKKRLLEPLGLVEEAAKTAPGKKRPVE | |
| | QSPQEPDSSAGIGKSGAQPAKKRLNFGQTGDTESVPDPQPIGEPPAAPSG | |
| | VGSLTMASGGGAPVADNNEGADGVGSSSGNWHCDSQWLGDRVITTSTR | |
| | TWALPTYNNHLYKQISNSTSGGSSNDNAYFGYSTPWGYFDFNRFHCHFS | |
| | PRDWQRLINNNWGFRPKRLNFKLFNIQVKEVTDNNGVKTIANNLTSTVQVF | |
| | TDSDYQLPYVLGSAHEGCLPPFPADVFMIPQYGYLTLNDGSQAVGRSSFY | |
| | CLEYFPSQMLRTGNNFQFSYEFENVPFHSSYAHSQSLDRLMNPLIDQYLY | |
| | YLSKTINGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGSLIFG | |
| | KQGTGHDNVDADKVMITNEEEIKTTNPVATESYGTVATNKQSAYAQAQTG | |
| | AVQNQGVLPGMVWQDRDVYLQGPIWAKIPHTDGNFHPSPLMGGFGMKH | |
| | PPPQILIKNTPVPADPPTAFNKDKLNSFITQYSTGQVSVEIEWELQKENSKR | |
| | WNPEIQYTSNYYKSNNVEFAVNTEGVYSEPRPIGTRYLTRNL | |
| V54 (amino | MAADGYLPDWLEDNLSEGIREWWALKPGAPQPKANQQHQDNARGLVLP | 349 |
| acid) | GYKYLGPGNGLDKGEPVNAADAAALEHDKAYDQQLKAGDNPYLKYNHAD | |
| | AEFQERLKEDTSFGGNLGRAVFQAKKRLLEPLGLVEEAAKTAPGKKRPVE | |
| | QSPQEPDSSAGIGKSGAQPAKKRLNFGQTGDTESVPDPQPIGEPPAAPSG | |
| | VGSLTMASGGGAPVADNNEGADGVGSSSGNWHCDSQWLGDRVITTSTR | |
| | TWALPTYNNHLYKQISNSTSGGSSNDNAYFGYSTPWGYFDFNRFHCHFS | |
| | PRDWQRLINNNWGFRPKRLNFKLFNIQVKEVTDNNGVKTIANNLTSTVQVF | |
| | TDSDYQLPYVLGSAHEGCLPPFPADVFMIPQYGYLTLNDGSQAVGRSSFY | |
| | CLEYFPSQMLRTGNNFQFSYEFENVPFHSSYAHSQSLDRLMNPLIDQYLY | |
| | YLSKTINGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGSLIFG | |
| | KQGTGHDNVDADKVMITNEEEIKTTNPVATEAYGQVATNKQSAYAQAQTG | |
| | AVQSQGILPGMVWQDRDVYLQGPIWAKIPHTDGNFHPSPLMGGFGMKHP | |
| | PPQILIKNTPVPADPPTAFNKDKLNSFITQYSTGQVSVEIEWELQKENSKRW | |
| | NPEIQYTSNYYKSNNVEFAVNTEGVYSEPRPIGTRYLTRNL | |
| V55 (amino | MAADGYLPDWLEDNLSEGIREWWALKPGAPQPKANQQHQDNARGLVLP | 350 |
| acid) | GYKYLGPGNGLDKGEPVNAADAAALEHDKAYDQQLKAGDNPYLKYNHAD | |
| | AEFQERLKEDTSFGGNLGRAVFQAKKRLLEPLGLVEEAAKTAPGKKRPVE | |
| | QSPQEPDSSAGIGKSGAQPAKKRLNFGQTGDTESVPDPQPIGEPPAAPSG | |
| | VGSLTMASGGGAPVADNNEGADGVGSSSGNWHCDSQWLGDRVITTSTR | |
| | TWALPTYNNHLYKQISNSTSGGSSNDNAYFGYSTPWGYFDFNRFHCHFS | |
| | PRDWQRLINNNWGFRPKRLNFKLFNIQVKEVTDNNGVKTIANNLTSTVQVF | |
| | TDSDYQLPYVLGSAHEGCLPPFPADVFMIPQYGYLTLNDGSQAVGRSSFY | |
| | CLEYFPSQMLRTGNNFQFSYEFENVPFHSSYAHSQSLDRLMNPLIDQYLY | |
| | YLSKTINGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGSLIFG | |
| | KQGTGHDNVDADKVMITNEEEIKTTNPVATEAYGTVATNHQSAYAQAQTG | |
| | AVQNQGVLPGMVWQDRDVYLQGPIWAKIPHTDGNFHPSPLMGGFGMKH | |
| | PPPQILIKNTPVPADPPTAFNKDKLNSFITQYSTGQVSVEIEWELQKENSKR | |
| | WNPEIQYTSNYYKSNNVEFAVNTEGVYSEPRPIGTRYLTRNL | |
| V56 (amino | MAADGYLPDWLEDNLSEGIREWWALKPGAPQPKANQQHQDNARGLVLP | 351 |
| acid) | GYKYLGPGNGLDKGEPVNAADAAALEHDKAYDQQLKAGDNPYLKYNHAD | |
| | AEFQERLKEDTSFGGNLGRAVFQAKKRLLEPLGLVEEAAKTAPGKKRPVE | |
| | QSPQEPDSSAGIGKSGAQPAKKRLNFGQTGDTESVPDPQPIGEPPAAPSG | |
| | VGSLTMASGGGAPVADNNEGADGVGSSSGNWHCDSQWLGDRVITTSTR | |
| | TWALPTYNNHLYKQISNSTSGGSSNDNAYFGYSTPWGYFDFNRFHCHFS | |
| | PRDWQRLINNNWGFRPKRLNFKLFNIQVKEVTDNNGVKTIANNLTSTVQVF | |
| | TDSDYQLPYVLGSAHEGCLPPFPADVFMIPQYGYLTLNDGSQAVGRSSFY | |
| | CLEYFPSQMLRTGNNFQFSYEFENVPFHSSYAHSQSLDRLMNPLIDQYLY | |
| | YLSKTINGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGSLIFG | |
| | KQGTGRDNVDADKVMITNEEEIKTTNPVATEAYGTVATNKQSAYAQAQTG | |
| | ALQNQGILPGMVWQDRDVYLQGPIWAKIPHTDGNFHPSPLMGGFGMKHP | |
| | PPQILIKNTPVPADPPTAFNKDKLNSFITQYSTGQVSVEIEWELQKENSKRW | |
| | NPEIQYTSNYYKSNNVEFAVNTEGVYSEPRPIGTRYLTRNL | |
| V57 (amino | MAADGYLPDWLEDNLSEGIREWWALKPGAPQPKANQQHQDNARGLVLP | 352 |
| acid) | GYKYLGPGNGLDKGEPVNAADAAALEHDKAYDQQLKAGDNPYLKYNHAD | |
| | AEFQERLKEDTSFGGNLGRAVFQAKKRLLEPLGLVEEAAKTAPGKKRPVE | |
| | QSPQEPDSSAGIGKSGAQPAKKRLNFGQTGDTESVPDPQPIGEPPAAPSG | |
| | VGSLTMASGGGAPVADNNEGADGVGSSSGNWHCDSQWLGDRVITTSTR | |
| | TWALPTYNNHLYKQISNSTSGGSSNDNAYFGYSTPWGYFDFNRFHCHFS | |
| | PRDWQRLINNNWGFRPKRLNFKLFNIQVKEVTDNNGVKTIANNLTSTVQVF | |
| | TDSDYQLPYVLGSAHEGCLPPFPADVFMIPQYGYLTLNDGSQAVGRSSFY | |
| | CLEYFPSQMLRTGNNFQFSYEFENVPFHSSYAHSQSLDRLMNPLIDQYLY | |
| | YLSKTINGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGSLIFG | |
| | KQGTGHDNVDADKVMITNEEEIKTTNPVATEAYGTVATNHQSAYAQAQTG | |
| | ALQNQGILPGMVWQDRDVYLQGPIWAKIPHTDGNFHPSPLMGGFGMKHP | |
| | PPQILIKNTPVPADPPTAFNKDKLNSFITQYSTGQVSVEIEWELQKENSKRW | |
| | NPEIQYTSNYYKSNNVEFAVNTEGVYSEPRPIGTRYLTRNL | |
| V58 (amino | MAADGYLPDWLEDNLSEGIREWWALKPGAPQPKANQQHQDNARGLVLP | 353 |
| acid) | GYKYLGPGNGLDKGEPVNAADAAALEHDKAYDQQLKAGDNPYLKYNHAD | |
| | AEFQERLKEDTSFGGNLGRAVFQAKKRLLEPLGLVEEAAKTAPGKKRPVE | |
| | QSPQEPDSSAGIGKSGAQPAKKRLNFGQTGDTESVPDPQPIGEPPAAPSG | |
| | VGSLTMASGGGAPVADNNEGADGVGSSSGNWHCDSQWLGDRVITTSTR | |
| | TWALPTYNNHLYKQISNSTSGGSSNDNAYFGYSTPWGYFDFNRFHCHFS | |
| | PRDWQRLINNNWGFRPKRLNFKLFNIQVKEVTDNNGVKTIANNLTSTVQVF | |
| | TDSDYQLPYVLGSAHEGCLPPFPADVFMIPQYGYLTLNDGSQAVGRSSFY | |
| | CLEYFPSQMLRTGNNFQFSYEFENVPFHSSYAHSQSLDRLMNPLIDQYLY | |
| | YLSKTINGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGSLIFG | |
| | KQGTGHDNVDADKVMITNEEEIKTTNPVATESYGQVATNKQSAYAQAQTG | |
| | AVQSQGVLPGMVWQDRDVYLQGPIWAKIPHTDGNFHPSPLMGGFGMKH | |
| | PPPQILIKNTPVPADPPTAFNKDKLNSFITQYSTGQVSVEIEWELQKENSKR | |
| | WNPEIQYTSNYYKSNNVEFAVNTEGVYSEPRPIGTRYLTRNL | |
| V59 (amino | MAADGYLPDWLEDNLSEGIREWWALKPGAPQPKANQQHQDNARGLVLP | 354 |
| acid) | GYKYLGPGNGLDKGEPVNAADAAALEHDKAYDQQLKAGDNPYLKYNHAD | |
| | AEFQERLKEDTSFGGNLGRAVFQAKKRLLEPLGLVEEAAKTAPGKKRPVE | |
| | QSPQEPDSSAGIGKSGAQPAKKRLNFGQTGDTESVPDPQPIGEPPAAPSG | |
| | VGSLTMASGGGAPVADNNEGADGVGSSSGNWHCDSQWLGDRVITTSTR | |
| | TWALPTYNNHLYKQISNSTSGGSSNDNAYFGYSTPWGYFDFNRFHCHFS | |
| | PRDWQRLINNNWGFRPKRLNFKLFNIQVKEVTDNNGVKTIANNLTSTVQVF | |
| | TDSDYQLPYVLGSAHEGCLPPFPADVFMIPQYGYLTLNDGSQAVGRSSFY | |
| | CLEYFPSQMLRTGNNFQFSYEFENVPFHSSYAHSQSLDRLMNPLIDQYLY | |
| | YLSKTINGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGSLIFG | |
| | KQGTGHDNVDADKVMITNEEEIKTTNPVATESYGTVATNHQSAYAQAQTG | |
| | ALQNQGVLPGMVWQDRDVYLQGPIWAKIPHTDGNFHPSPLMGGFGMKH | |
| | PPPQILIKNTPVPADPPTAFNKDKLNSFITQYSTGQVSVEIEWELQKENSKR | |
| | WNPEIQYTSNYYKSNNVEFAVNTEGVYSEPRPIGTRYLTRNL | |
| V60 (amino | MAADGYLPDWLEDNLSEGIREWWALKPGAPQPKANQQHQDNARGLVLP | 355 |
| acid) | GYKYLGPGNGLDKGEPVNAADAAALEHDKAYDQQLKAGDNPYLKYNHAD | |
| | AEFQERLKEDTSFGGNLGRAVFQAKKRLLEPLGLVEEAAKTAPGKKRPVE | |
| | QSPQEPDSSAGIGKSGAQPAKKRLNFGQTGDTESVPDPQPIGEPPAAPSG | |
| | VGSLTMASGGGAPVADNNEGADGVGSSSGNWHCDSQWLGDRVITTSTR | |
| | TWALPTYNNHLYKQISNSTSGGSSNDNAYFGYSTPWGYFDFNRFHCHFS | |
| | PRDWQRLINNNWGFRPKRLNFKLFNIQVKEVTDNNGVKTIANNLTSTVQVF | |
| | TDSDYQLPYVLGSAHEGCLPPFPADVFMIPQYGYLTLNDGSQAVGRSSFY | |
| | CLEYFPSQMLRTGNNFQFSYEFENVPFHSSYAHSQSLDRLMNPLIDQYLY | |
| | YLSKTINGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGSLIFG | |
| | KQGTGHDNVDADKVMITNEEEIKTTNPVATESYGTVATNHQSAYAQAQTG | |
| | ALQSQGILPGMVWQDRDVYLQGPIWAKIPHTDGNFHPSPLMGGFGMKHP | |
| | PPQILIKNTPVPADPPTAFNKDKLNSFITQYSTGQVSVEIEWELQKENSKRW | |
| | NPEIQYTSNYYKSNNVEFAVNTEGVYSEPRPIGTRYLTRNL | |
| V61 (amino | MAADGYLPDWLEDNLSEGIREWWALKPGAPQPKANQQHQDNARGLVLP | 356 |
| acid) | GYKYLGPGNGLDKGEPVNAADAAALEHDKAYDQQLKAGDNPYLKYNHAD | |
| | AEFQERLKEDTSFGGNLGRAVFQAKKRLLEPLGLVEEAAKTAPGKKRPVE | |
| | QSPQEPDSSAGIGKSGAQPAKKRLNFGQTGDTESVPDPQPIGEPPAAPSG | |
| | VGSLTMASGGGAPVADNNEGADGVGSSSGNWHCDSQWLGDRVITTSTR | |
| | TWALPTYNNHLYKQISNSTSGGSSNDNAYFGYSTPWGYFDFNRFHCHFS | |
| | PRDWQRLINNNWGFRPKRLNFKLFNIQVKEVTDNNGVKTIANNLTSTVQVF | |
| | TDSDYQLPYVLGSAHEGCLPPFPADVFMIPQYGYLTLNDGSQAVGRSSFY | |
| | CLEYFPSQMLRTGNNFQFSYEFENVPFHSSYAHSQSLDRLMNPLIDQYLY | |
| | YLSKTINGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGSLIFG | |
| | KQGTGHDNVDADKVMITNEEEIKTTNPVATEAYGQVATNHQSAYAQAQTG | |
| | ALQSQGILPGMVWQDRDVYLQGPIWAKIPHTDGNFHPSPLMGGFGMKHP | |
| | PPQILIKNTPVPADPPTAFNKDKLNSFITQYSTGQVSVEIEWELQKENSKRW | |
| | NPEIQYTSNYYKSNNVEFAVNTEGVYSEPRPIGTRYLTRNL | |
| V62 (amino | MAADGYLPDWLEDNLSEGIREWWALKPGAPQPKANQQHQDNARGLVLP | 357 |
| acid) | GYKYLGPGNGLDKGEPVNAADAAALEHDKAYDQQLKAGDNPYLKYNHAD | |
| | AEFQERLKEDTSFGGNLGRAVFQAKKRLLEPLGLVEEAAKTAPGKKRPVE | |
| | QSPQEPDSSAGIGKSGAQPAKKRLNFGQTGDTESVPDPQPIGEPPAAPSG | |
| | VGSLTMASGGGAPVADNNEGADGVGSSSGNWHCDSQWLGDRVITTSTR | |
| | TWALPTYNNHLYKQISNSTSGGSSNDNAYFGYSTPWGYFDFNRFHCHFS | |
| | PRDWQRLINNNWGFRPKRLNFKLFNIQVKEVTDNNGVKTIANNLTSTVQVF | |
| | TDSDYQLPYVLGSAHEGCLPPFPADVFMIPQYGYLTLNDGSQAVGRSSFY | |
| | CLEYFPSQMLRTGNNFQFSYEFENVPFHSSYAHSQSLDRLMNPLIDQYLY | |
| | YLSKTINGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGSLIFG | |
| | KQGTGRDNVDADKVMITNEEEIKTTNPVATEAYGTVATNHQSAYAQAQTG | |
| | ALQNQGVLPGMVWQDRDVYLQGPIWAKIPHTDGNFHPSPLMGGFGMKH | |
| | PPPQILIKNTPVPADPPTAFNKDKLNSFITQYSTGQVSVEIEWELQKENSKR | |
| | WNPEIQYTSNYYKSNNVEFAVNTEGVYSEPRPIGTRYLTRNL | |
| V63 (amino | MAADGYLPDWLEDNLSEGIREWWALKPGAPQPKANQQHQDNARGLVLP | 358 |
| acid) | GYKYLGPGNGLDKGEPVNAADAAALEHDKAYDQQLKAGDNPYLKYNHAD | |
| | AEFQERLKEDTSFGGNLGRAVFQAKKRLLEPLGLVEEAAKTAPGKKRPVE | |
| | QSPQEPDSSAGIGKSGAQPAKKRLNFGQTGDTESVPDPQPIGEPPAAPSG | |
| | VGSLTMASGGGAPVADNNEGADGVGSSSGNWHCDSQWLGDRVITTSTR | |
| | TWALPTYNNHLYKQISNSTSGGSSNDNAYFGYSTPWGYFDFNRFHCHFS | |
| | PRDWQRLINNNWGFRPKRLNFKLFNIQVKEVTDNNGVKTIANNLTSTVQVF | |
| | TDSDYQLPYVLGSAHEGCLPPFPADVFMIPQYGYLTLNDGSQAVGRSSFY | |
| | CLEYFPSQMLRTGNNFQFSYEFENVPFHSSYAHSQSLDRLMNPLIDQYLY | |
| | YLSKTINGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGSLIFG | |
| | KQGTGRDNVDADKVMITNEEEIKTTNPVATESYGTVATNKQSAQAQAQTG | |
| | ALQSQGVLPGMVWQDRDVYLQGPIWAKIPHTDGNFHPSPLMGGFGMKHP | |
| | PPQILIKNTPVPADPPTAFNKDKLNSFITQYSTGQVSVEIEWELQKENSKRW | |
| | NPEIQYTSNYYKSNNVEFAVNTEGVYSEPRPIGTRYLTRNL | |
| V64 (amino | MAADGYLPDWLEDNLSEGIREWWALKPGAPQPKANQQHQDNARGLVLP | 359 |
| acid) | GYKYLGPGNGLDKGEPVNAADAAALEHDKAYDQQLKAGDNPYLKYNHAD | |
| | AEFQERLKEDTSFGGNLGRAVFQAKKRLLEPLGLVEEAAKTAPGKKRPVE | |
| | QSPQEPDSSAGIGKSGAQPAKKRLNFGQTGDTESVPDPQPIGEPPAAPSG | |
| | VGSLTMASGGGAPVADNNEGADGVGSSSGNWHCDSQWLGDRVITTSTR | |
| | TWALPTYNNHLYKQISNSTSGGSSNDNAYFGYSTPWGYFDFNRFHCHFS | |
| | PRDWQRLINNNWGFRPKRLNFKLFNIQVKEVTDNNGVKTIANNLTSTVQVF | |
| | TDSDYQLPYVLGSAHEGCLPPFPADVFMIPQYGYLTLNDGSQAVGRSSFY | |
| | CLEYFPSQMLRTGNNFQFSYEFENVPFHSSYAHSQSLDRLMNPLIDQYLY | |
| | YLSKTINGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGSLIFG | |
| | KQGTGHDNVDADKVMITNEEEIKTTNPVATESYGTVATNKQSAYAQAQTG | |
| | AVQSQGILPGMVWQDRDVYLQGPIWAKIPHTDGNFHPSPLMGGFGMKHP | |
| | PPQILIKNTPVPADPPTAFNKDKLNSFITQYSTGQVSVEIEWELQKENSKRW | |
| | NPEIQYTSNYYKSNNVEFAVNTEGVYSEPRPIGTRYLTRNL | |
| V65 (amino | MAADGYLPDWLEDNLSEGIREWWALKPGAPQPKANQQHQDNARGLVLP | 360 |
| acid) | GYKYLGPGNGLDKGEPVNAADAAALEHDKAYDQQLKAGDNPYLKYNHAD | |
| | AEFQERLKEDTSFGGNLGRAVFQAKKRLLEPLGLVEEAAKTAPGKKRPVE | |
| | QSPQEPDSSAGIGKSGAQPAKKRLNFGQTGDTESVPDPQPIGEPPAAPSG | |
| | VGSLTMASGGGAPVADNNEGADGVGSSSGNWHCDSQWLGDRVITTSTR | |
| | TWALPTYNNHLYKQISNSTSGGSSNDNAYFGYSTPWGYFDFNRFHCHFS | |
| | PRDWQRLINNNWGFRPKRLNFKLFNIQVKEVTDNNGVKTIANNLTSTVQVF | |
| | TDSDYQLPYVLGSAHEGCLPPFPADVFMIPQYGYLTLNDGSQAVGRSSFY | |
| | CLEYFPSQMLRTGNNFQFSYEFENVPFHSSYAHSQSLDRLMNPLIDQYLY | |
| | YLSKTINGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGSLIFG | |
| | KQGTGRDNVDADKVMITNEEEIKTTNPVATEAYGTVATNHQSAQAQAQTG | |
| | ALQSQGVLPGMVWQDRDVYLQGPIWAKIPHTDGNFHPSPLMGGFGMKHP | |
| | PPQILIKNTPVPADPPTAFNKDKLNSFITQYSTGQVSVEIEWELQKENSKRW | |
| | NPEIQYTSNYYKSNNVEFAVNTEGVYSEPRPIGTRYLTRNL | |
| V66 (amino | MAADGYLPDWLEDNLSEGIREWWALKPGAPQPKANQQHQDNARGLVLP | 361 |
| acid) | GYKYLGPGNGLDKGEPVNAADAAALEHDKAYDQQLKAGDNPYLKYNHAD | |
| | AEFQERLKEDTSFGGNLGRAVFQAKKRLLEPLGLVEEAAKTAPGKKRPVE | |
| | QSPQEPDSSAGIGKSGAQPAKKRLNFGQTGDTESVPDPQPIGEPPAAPSG | |
| | VGSLTMASGGGAPVADNNEGADGVGSSSGNWHCDSQWLGDRVITTSTR | |
| | TWALPTYNNHLYKQISNSTSGGSSNDNAYFGYSTPWGYFDFNRFHCHFS | |
| | PRDWQRLINNNWGFRPKRLNFKLFNIQVKEVTDNNGVKTIANNLTSTVQVF | |
| | TDSDYQLPYVLGSAHEGCLPPFPADVFMIPQYGYLTLNDGSQAVGRSSFY | |
| | CLEYFPSQMLRTGNNFQFSYEFENVPFHSSYAHSQSLDRLMNPLIDQYLY | |
| | YLSKTINGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGSLIFG | |
| | KQGTGRDNVDADKVMITNEEEIKTTNPVATEAYGTVATNHQSAYAQAQTG | |
| | AVQSQGVLPGMVWQDRDVYLQGPIWAKIPHTDGNFHPSPLMGGFGMKH | |
| | PPPQILIKNTPVPADPPTAFNKDKLNSFITQYSTGQVSVEIEWELQKENSKR | |
| | WNPEIQYTSNYYKSNNVEFAVNTEGVYSEPRPIGTRYLTRNL | |
| V67 (amino | MAADGYLPDWLEDNLSEGIREWWALKPGAPQPKANQQHQDNARGLVLP | 362 |
| acid) | GYKYLGPGNGLDKGEPVNAADAAALEHDKAYDQQLKAGDNPYLKYNHAD | |
| | AEFQERLKEDTSFGGNLGRAVFQAKKRLLEPLGLVEEAAKTAPGKKRPVE | |
| | QSPQEPDSSAGIGKSGAQPAKKRLNFGQTGDTESVPDPQPIGEPPAAPSG | |
| | VGSLTMASGGGAPVADNNEGADGVGSSSGNWHCDSQWLGDRVITTSTR | |
| | TWALPTYNNHLYKQISNSTSGGSSNDNAYFGYSTPWGYFDFNRFHCHFS | |
| | PRDWQRLINNNWGFRPKRLNFKLFNIQVKEVTDNNGVKTIANNLTSTVQVF | |
| | TDSDYQLPYVLGSAHEGCLPPFPADVFMIPQYGYLTLNDGSQAVGRSSFY | |
| | CLEYFPSQMLRTGNNFQFSYEFENVPFHSSYAHSQSLDRLMNPLIDQYLY | |
| | YLSKTINGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGSLIFG | |
| | KQGTGRDNVDADKVMITNEEEIKTTNPVATESYGTVATNKQSAYAQAQTG | |
| | ALQSQGILPGMVWQDRDVYLQGPIWAKIPHTDGNFHPSPLMGGFGMKHP | |
| | PPQILIKNTPVPADPPTAFNKDKLNSFITQYSTGQVSVEIEWELQKENSKRW | |
| | NPEIQYTSNYYKSNNVEFAVNTEGVYSEPRPIGTRYLTRNL | |
| V68 (amino | MAADGYLPDWLEDNLSEGIREWWALKPGAPQPKANQQHQDNARGLVLP | 363 |
| acid) | GYKYLGPGNGLDKGEPVNAADAAALEHDKAYDQQLKAGDNPYLKYNHAD | |
| | AEFQERLKEDTSFGGNLGRAVFQAKKRLLEPLGLVEEAAKTAPGKKRPVE | |
| | QSPQEPDSSAGIGKSGAQPAKKRLNFGQTGDTESVPDPQPIGEPPAAPSG | |
| | VGSLTMASGGGAPVADNNEGADGVGSSSGNWHCDSQWLGDRVITTSTR | |
| | TWALPTYNNHLYKQISNSTSGGSSNDNAYFGYSTPWGYFDFNRFHCHFS | |
| | PRDWQRLINNNWGFRPKRLNFKLFNIQVKEVTDNNGVKTIANNLTSTVQVF | |
| | TDSDYQLPYVLGSAHEGCLPPFPADVFMIPQYGYLTLNDGSQAVGRSSFY | |
| | CLEYFPSQMLRTGNNFQFSYEFENVPFHSSYAHSQSLDRLMNPLIDQYLY | |
| | YLSKTINGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGSLIFG | |
| | KQGTGHDNVDADKVMITNEEEIKTTNPVATESYGQVATNHQSAYAQAQTG | |
| | ALQSQGVLPGMVWQDRDVYLQGPIWAKIPHTDGNFHPSPLMGGFGMKHP | |
| | PPQILIKNTPVPADPPTAFNKDKLNSFITQYSTGQVSVEIEWELQKENSKRW | |
| | NPEIQYTSNYYKSNNVEFAVNTEGVYSEPRPIGTRYLTRNL | |
| V69 (amino | MAADGYLPDWLEDNLSEGIREWWALKPGAPQPKANQQHQDNARGLVLP | 364 |
| acid) | GYKYLGPGNGLDKGEPVNAADAAALEHDKAYDQQLKAGDNPYLKYNHAD | |
| | AEFQERLKEDTSFGGNLGRAVFQAKKRLLEPLGLVEEAAKTAPGKKRPVE | |
| | QSPQEPDSSAGIGKSGAQPAKKRLNFGQTGDTESVPDPQPIGEPPAAPSG | |
| | VGSLTMASGGGAPVADNNEGADGVGSSSGNWHCDSQWLGDRVITTSTR | |
| | TWALPTYNNHLYKQISNSTSGGSSNDNAYFGYSTPWGYFDFNRFHCHFS | |
| | PRDWQRLINNNWGFRPKRLNFKLFNIQVKEVTDNNGVKTIANNLTSTVQVF | |
| | TDSDYQLPYVLGSAHEGCLPPFPADVFMIPQYGYLTLNDGSQAVGRSSFY | |
| | CLEYFPSQMLRTGNNFQFSYEFENVPFHSSYAHSQSLDRLMNPLIDQYLY | |
| | YLSKTINGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGSLIFG | |
| | KQGTGHDNVDADKVMITNEEEIKTTNPVATEAYGQVATNHQSAYAQAQTG | |
| | ALQNQGVLPGMVWQDRDVYLQGPIWAKIPHTDGNFHPSPLMGGFGMKH | |
| | PPPQILIKNTPVPADPPTAFNKDKLNSFITQYSTGQVSVEIEWELQKENSKR | |
| | WNPEIQYTSNYYKSNNVEFAVNTEGVYSEPRPIGTRYLTRNL | |
| V70 (amino | MAADGYLPDWLEDNLSEGIREWWALKPGAPQPKANQQHQDNARGLVLP | 365 |
| acid) | GYKYLGPGNGLDKGEPVNAADAAALEHDKAYDQQLKAGDNPYLKYNHAD | |
| | AEFQERLKEDTSFGGNLGRAVFQAKKRLLEPLGLVEEAAKTAPGKKRPVE | |
| | QSPQEPDSSAGIGKSGAQPAKKRLNFGQTGDTESVPDPQPIGEPPAAPSG | |
| | VGSLTMASGGGAPVADNNEGADGVGSSSGNWHCDSQWLGDRVITTSTR | |
| | TWALPTYNNHLYKQISNSTSGGSSNDNAYFGYSTPWGYFDFNRFHCHFS | |
| | PRDWQRLINNNWGFRPKRLNFKLFNIQVKEVTDNNGVKTIANNLTSTVQVF | |
| | TDSDYQLPYVLGSAHEGCLPPFPADVFMIPQYGYLTLNDGSQAVGRSSFY | |
| | CLEYFPSQMLRTGNNFQFSYEFENVPFHSSYAHSQSLDRLMNPLIDQYLY | |
| | YLSKTINGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGSLIFG | |
| | KQGTGHDNVDADKVMITNEEEIKTTNPVATESYGTVATNKQSAYAQAQTG | |
| | ALQNQGILPGMVWQDRDVYLQGPIWAKIPHTDGNFHPSPLMGGFGMKHP | |
| | PPQILIKNTPVPADPPTAFNKDKLNSFITQYSTGQVSVEIEWELQKENSKRW | |
| | NPEIQYTSNYYKSNNVEFAVNTEGVYSEPRPIGTRYLTRNL | |
| V71 (amino | MAADGYLPDWLEDNLSEGIREWWALKPGAPQPKANQQHQDNARGLVLP | 366 |
| acid) | GYKYLGPGNGLDKGEPVNAADAAALEHDKAYDQQLKAGDNPYLKYNHAD | |
| | AEFQERLKEDTSFGGNLGRAVFQAKKRLLEPLGLVEEAAKTAPGKKRPVE | |
| | QSPQEPDSSAGIGKSGAQPAKKRLNFGQTGDTESVPDPQPIGEPPAAPSG | |
| | VGSLTMASGGGAPVADNNEGADGVGSSSGNWHCDSQWLGDRVITTSTR | |
| | TWALPTYNNHLYKQISNSTSGGSSNDNAYFGYSTPWGYFDFNRFHCHFS | |
| | PRDWQRLINNNWGFRPKRLNFKLFNIQVKEVTDNNGVKTIANNLTSTVQVF | |
| | TDSDYQLPYVLGSAHEGCLPPFPADVFMIPQYGYLTLNDGSQAVGRSSFY | |
| | CLEYFPSQMLRTGNNFQFSYEFENVPFHSSYAHSQSLDRLMNPLIDQYLY | |
| | YLSKTINGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGSLIFG | |
| | KQGTGHDNVDADKVMITNEEEIKTTNPVATESYGTVATNKQSAQAQAQTG | |
| | WLQSQGVLPGMVWQDRDVYLQGPIWAKIPHTDGNFHPSPLMGGFGMKH | |
| | PPPQILIKNTPVPADPPTAFNKDKLNSFITQYSTGQVSVEIEWELQKENSKR | |
| | WNPEIQYTSNYYKSNNVEFAVNTEGVYSEPRPIGTRYLTRNL | |
| V72 (amino | MAADGYLPDWLEDNLSEGIREWWALKPGAPQPKANQQHQDNARGLVLP | 367 |
| acid) | GYKYLGPGNGLDKGEPVNAADAAALEHDKAYDQQLKAGDNPYLKYNHAD | |
| | AEFQERLKEDTSFGGNLGRAVFQAKKRLLEPLGLVEEAAKTAPGKKRPVE | |
| | QSPQEPDSSAGIGKSGAQPAKKRLNFGQTGDTESVPDPQPIGEPPAAPSG | |
| | VGSLTMASGGGAPVADNNEGADGVGSSSGNWHCDSQWLGDRVITTSTR | |
| | TWALPTYNNHLYKQISNSTSGGSSNDNAYFGYSTPWGYFDFNRFHCHFS | |
| | PRDWQRLINNNWGFRPKRLNFKLFNIQVKEVTDNNGVKTIANNLTSTVQVF | |
| | TDSDYQLPYVLGSAHEGCLPPFPADVFMIPQYGYLTLNDGSQAVGRSSFY | |
| | CLEYFPSQMLRTGNNFQFSYEFENVPFHSSYAHSQSLDRLMNPLIDQYLY | |
| | YLSKTINGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGSLIFG | |
| | KQGTGHDNVDADKVMITNEEEIKTTNPVATESYGTVATNHQSAYAQAQTG | |
| | AVQSQGVLPGMVWQDRDVYLQGPIWAKIPHTDGNFHPSPLMGGFGMKH | |
| | PPPQILIKNTPVPADPPTAFNKDKLNSFITQYSTGQVSVEIEWELQKENSKR | |
| | WNPEIQYTSNYYKSNNVEFAVNTEGVYSEPRPIGTRYLTRNL | |
| V73 (amino | MAADGYLPDWLEDNLSEGIREWWALKPGAPQPKANQQHQDNARGLVLP | 368 |
| acid) | GYKYLGPGNGLDKGEPVNAADAAALEHDKAYDQQLKAGDNPYLKYNHAD | |
| | AEFQERLKEDTSFGGNLGRAVFQAKKRLLEPLGLVEEAAKTAPGKKRPVE | |
| | QSPQEPDSSAGIGKSGAQPAKKRLNFGQTGDTESVPDPQPIGEPPAAPSG | |
| | VGSLTMASGGGAPVADNNEGADGVGSSSGNWHCDSQWLGDRVITTSTR | |
| | TWALPTYNNHLYKQISNSTSGGSSNDNAYFGYSTPWGYFDFNRFHCHFS | |
| | PRDWQRLINNNWGFRPKRLNFKLFNIQVKEVTDNNGVKTIANNLTSTVQVF | |
| | TDSDYQLPYVLGSAHEGCLPPFPADVFMIPQYGYLTLNDGSQAVGRSSFY | |
| | CLEYFPSQMLRTGNNFQFSYEFENVPFHSSYAHSQSLDRLMNPLIDQYLY | |
| | YLSKTINGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGSLIFG | |
| | KQGTGHDNVDADKVMITNEEEIKTTNPVATEAYGQVATNKQSAYAQAQTG | |
| | ALQNQGILPGMVWQDRDVYLQGPIWAKIPHTDGNFHPSPLMGGFGMKHP | |
| | PPQILIKNTPVPADPPTAFNKDKLNSFITQYSTGQVSVEIEWELQKENSKRW | |
| | NPEIQYTSNYYKSNNVEFAVNTEGVYSEPRPIGTRYLTRNL | |
| V74 (amino | MAADGYLPDWLEDNLSEGIREWWALKPGAPQPKANQQHQDNARGLVLP | 369 |
| acid) | GYKYLGPGNGLDKGEPVNAADAAALEHDKAYDQQLKAGDNPYLKYNHAD | |
| | AEFQERLKEDTSFGGNLGRAVFQAKKRLLEPLGLVEEAAKTAPGKKRPVE | |
| | QSPQEPDSSAGIGKSGAQPAKKRLNFGQTGDTESVPDPQPIGEPPAAPSG | |
| | VGSLTMASGGGAPVADNNEGADGVGSSSGNWHCDSQWLGDRVITTSTR | |
| | TWALPTYNNHLYKQISNSTSGGSSNDNAYFGYSTPWGYFDFNRFHCHFS | |
| | PRDWQRLINNNWGFRPKRLNFKLFNIQVKEVTDNNGVKTIANNLTSTVQVF | |
| | TDSDYQLPYVLGSAHEGCLPPFPADVFMIPQYGYLTLNDGSQAVGRSSFY | |
| | CLEYFPSQMLRTGNNFQFSYEFENVPFHSSYAHSQSLDRLMNPLIDQYLY | |
| | YLSKTINGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGSLIFG | |
| | KQGTGRDNVDADKVMITNEEEIKTTNPVATEAYGQVATNKQSAYAQAQTG | |
| | AVQSQGVLPGMVWQDRDVYLQGPIWAKIPHTDGNFHPSPLMGGFGMKH | |
| | PPPQILIKNTPVPADPPTAFNKDKLNSFITQYSTGQVSVEIEWELQKENSKR | |
| | WNPEIQYTSNYYKSNNVEFAVNTEGVYSEPRPIGTRYLTRNL | |
| V75 (amino | MAADGYLPDWLEDNLSEGIREWWALKPGAPQPKANQQHQDNARGLVLP | 370 |
| acid) | GYKYLGPGNGLDKGEPVNAADAAALEHDKAYDQQLKAGDNPYLKYNHAD | |
| | AEFQERLKEDTSFGGNLGRAVFQAKKRLLEPLGLVEEAAKTAPGKKRPVE | |
| | QSPQEPDSSAGIGKSGAQPAKKRLNFGQTGDTESVPDPQPIGEPPAAPSG | |
| | VGSLTMASGGGAPVADNNEGADGVGSSSGNWHCDSQWLGDRVITTSTR | |
| | TWALPTYNNHLYKQISNSTSGGSSNDNAYFGYSTPWGYFDFNRFHCHFS | |
| | PRDWQRLINNNWGFRPKRLNFKLFNIQVKEVTDNNGVKTIANNLTSTVQVF | |
| | TDSDYQLPYVLGSAHEGCLPPFPADVFMIPQYGYLTLNDGSQAVGRSSFY | |
| | CLEYFPSQMLRTGNNFQFSYEFENVPFHSSYAHSQSLDRLMNPLIDQYLY | |
| | YLSKTINGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGSLIFG | |
| | KQGTGHDNVDADKVMITNEEEIKTTNPVATESYGTVATNHQSAQAQAQTG | |
| | ALQSQGVLPGMVWQDRDVYLQGPIWAKIPHTDGNFHPSPLMGGFGMKHP | |
| | PPQILIKNTPVPADPPTAFNKDKLNSFITQYSTGQVSVEIEWELQKENSKRW | |
| | NPEIQYTSNYYKSNNVEFAVNTEGVYSEPRPIGTRYLTRNL | |
| V76 (amino | MAADGYLPDWLEDNLSEGIREWWALKPGAPQPKANQQHQDNARGLVLP | 371 |
| acid) | GYKYLGPGNGLDKGEPVNAADAAALEHDKAYDQQLKAGDNPYLKYNHAD | |
| | AEFQERLKEDTSFGGNLGRAVFQAKKRLLEPLGLVEEAAKTAPGKKRPVE | |
| | QSPQEPDSSAGIGKSGAQPAKKRLNFGQTGDTESVPDPQPIGEPPAAPSG | |
| | VGSLTMASGGGAPVADNNEGADGVGSSSGNWHCDSQWLGDRVITTSTR | |
| | TWALPTYNNHLYKQISNSTSGGSSNDNAYFGYSTPWGYFDFNRFHCHFS | |
| | PRDWQRLINNNWGFRPKRLNFKLFNIQVKEVTDNNGVKTIANNLTSTVQVF | |
| | TDSDYQLPYVLGSAHEGCLPPFPADVFMIPQYGYLTLNDGSQAVGRSSFY | |
| | CLEYFPSQMLRTGNNFQFSYEFENVPFHSSYAHSQSLDRLMNPLIDQYLY | |
| | YLSKTINGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGSLIFG | |
| | KQGTGHDNVDADKVMITNEEEIKTTNPVATESYGTVATNKQSAQAQAQTG | |
| | ALQSQGILPGMVWQDRDVYLQGPIWAKIPHTDGNFHPSPLMGGFGMKHP | |
| | PPQILIKNTPVPADPPTAFNKDKLNSFITQYSTGQVSVEIEWELQKENSKRW | |
| | NPEIQYTSNYYKSNNVEFAVNTEGVYSEPRPIGTRYLTRNL | |
| V77 (amino | MAADGYLPDWLEDNLSEGIREWWALKPGAPQPKANQQHQDNARGLVLP | 372 |
| acid) | GYKYLGPGNGLDKGEPVNAADAAALEHDKAYDQQLKAGDNPYLKYNHAD | |
| | AEFQERLKEDTSFGGNLGRAVFQAKKRLLEPLGLVEEAAKTAPGKKRPVE | |
| | QSPQEPDSSAGIGKSGAQPAKKRLNFGQTGDTESVPDPQPIGEPPAAPSG | |
| | VGSLTMASGGGAPVADNNEGADGVGSSSGNWHCDSQWLGDRVITTSTR | |
| | TWALPTYNNHLYKQISNSTSGGSSNDNAYFGYSTPWGYFDFNRFHCHFS | |
| | PRDWQRLINNNWGFRPKRLNFKLFNIQVKEVTDNNGVKTIANNLTSTVQVF | |
| | TDSDYQLPYVLGSAHEGCLPPFPADVFMIPQYGYLTLNDGSQAVGRSSFY | |
| | CLEYFPSQMLRTGNNFQFSYEFENVPFHSSYAHSQSLDRLMNPLIDQYLY | |
| | YLSKTINGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGSLIFG | |
| | KQGTGHDNVDADKVMITNEEEIKTTNPVATESYGTVATNKQSAYAQAQTG | |
| | AVQNQGILPGMVWQDRDVYLQGPIWAKIPHTDGNFHPSPLMGGFGMKHP | |
| | PPQILIKNTPVPADPPTAFNKDKLNSFITQYSTGQVSVEIEWELQKENSKRW | |
| | NPEIQYTSNYYKSNNVEFAVNTEGVYSEPRPIGTRYLTRNL | |
| V78 (amino | MAADGYLPDWLEDNLSEGIREWWALKPGAPQPKANQQHQDNARGLVLP | 373 |
| acid) | GYKYLGPGNGLDKGEPVNAADAAALEHDKAYDQQLKAGDNPYLKYNHAD | |
| | AEFQERLKEDTSFGGNLGRAVFQAKKRLLEPLGLVEEAAKTAPGKKRPVE | |
| | QSPQEPDSSAGIGKSGAQPAKKRLNFGQTGDTESVPDPQPIGEPPAAPSG | |
| | VGSLTMASGGGAPVADNNEGADGVGSSSGNWHCDSQWLGDRVITTSTR | |
| | TWALPTYNNHLYKQISNSTSGGSSNDNAYFGYSTPWGYFDFNRFHCHFS | |
| | PRDWQRLINNNWGFRPKRLNFKLFNIQVKEVTDNNGVKTIANNLTSTVQVF | |
| | TDSDYQLPYVLGSAHEGCLPPFPADVFMIPQYGYLTLNDGSQAVGRSSFY | |
| | CLEYFPSQMLRTGNNFQFSYEFENVPFHSSYAHSQSLDRLMNPLIDQYLY | |
| | YLSKTINGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGSLIFG | |
| | KQGTGRDNVDADKVMITNEEEIKTTNPVATEAYGQVATNKQSAYAQAQTG | |
| | ALQNQGILPGMVWQDRDVYLQGPIWAKIPHTDGNFHPSPLMGGFGMKHP | |
| | PPQILIKNTPVPADPPTAFNKDKLNSFITQYSTGQVSVEIEWELQKENSKRW | |
| | NPEIQYTSNYYKSNNVEFAVNTEGVYSEPRPIGTRYLTRNL | |
| V79 (amino | MAADGYLPDWLEDNLSEGIREWWALKPGAPQPKANQQHQDNARGLVLP | 374 |
| acid) | GYKYLGPGNGLDKGEPVNAADAAALEHDKAYDQQLKAGDNPYLKYNHAD | |
| | AEFQERLKEDTSFGGNLGRAVFQAKKRLLEPLGLVEEAAKTAPGKKRPVE | |
| | QSPQEPDSSAGIGKSGAQPAKKRLNFGQTGDTESVPDPQPIGEPPAAPSG | |
| | VGSLTMASGGGAPVADNNEGADGVGSSSGNWHCDSQWLGDRVITTSTR | |
| | TWALPTYNNHLYKQISNSTSGGSSNDNAYFGYSTPWGYFDFNRFHCHFS | |
| | PRDWQRLINNNWGFRPKRLNFKLFNIQVKEVTDNNGVKTIANNLTSTVQVF | |
| | TDSDYQLPYVLGSAHEGCLPPFPADVFMIPQYGYLTLNDGSQAVGRSSFY | |
| | CLEYFPSQMLRTGNNFQFSYEFENVPFHSSYAHSQSLDRLMNPLIDQYLY | |
| | YLSKTINGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGSLIFG | |
| | KQGTGRDNVDADKVMITNEEEIKTTNPVATESYGQVATNKQSAYAQAQTG | |
| | ALQNQGVLPGMVWQDRDVYLQGPIWAKIPHTDGNFHPSPLMGGFGMKH | |
| | PPPQILIKNTPVPADPPTAFNKDKLNSFITQYSTGQVSVEIEWELQKENSKR | |
| | WNPEIQYTSNYYKSNNVEFAVNTEGVYSEPRPIGTRYLTRNL | |
| V80 (amino | MAADGYLPDWLEDNLSEGIREWWALKPGAPQPKANQQHQDNARGLVLP | 375 |
| acid) | GYKYLGPGNGLDKGEPVNAADAAALEHDKAYDQQLKAGDNPYLKYNHAD | |
| | AEFQERLKEDTSFGGNLGRAVFQAKKRLLEPLGLVEEAAKTAPGKKRPVE | |
| | QSPQEPDSSAGIGKSGAQPAKKRLNFGQTGDTESVPDPQPIGEPPAAPSG | |
| | VGSLTMASGGGAPVADNNEGADGVGSSSGNWHCDSQWLGDRVITTSTR | |
| | TWALPTYNNHLYKQISNSTSGGSSNDNAYFGYSTPWGYFDFNRFHCHFS | |
| | PRDWQRLINNNWGFRPKRLNFKLFNIQVKEVTDNNGVKTIANNLTSTVQVF | |
| | TDSDYQLPYVLGSAHEGCLPPFPADVFMIPQYGYLTLNDGSQAVGRSSFY | |
| | CLEYFPSQMLRTGNNFQFSYEFENVPFHSSYAHSQSLDRLMNPLIDQYLY | |
| | YLSKTINGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGSLIFG | |
| | KQGTGRDNVDADKVMITNEEEIKTTNPVATESYGTVATNKQSAYAQAQTG | |
| | AVQNQGVLPGMVWQDRDVYLQGPIWAKIPHTDGNFHPSPLMGGFGMKH | |
| | PPPQILIKNTPVPADPPTAFNKDKLNSFITQYSTGQVSVEIEWELQKENSKR | |
| | WNPEIQYTSNYYKSNNVEFAVNTEGVYSEPRPIGTRYLTRNL | |
| V81 (amino | MAADGYLPDWLEDNLSEGIREWWALKPGAPQPKANQQHQDNARGLVLP | 376 |
| acid) | GYKYLGPGNGLDKGEPVNAADAAALEHDKAYDQQLKAGDNPYLKYNHAD | |
| | AEFQERLKEDTSFGGNLGRAVFQAKKRLLEPLGLVEEAAKTAPGKKRPVE | |
| | QSPQEPDSSAGIGKSGAQPAKKRLNFGQTGDTESVPDPQPIGEPPAAPSG | |
| | VGSLTMASGGGAPVADNNEGADGVGSSSGNWHCDSQWLGDRVITTSTR | |
| | TWALPTYNNHLYKQISNSTSGGSSNDNAYFGYSTPWGYFDFNRFHCHFS | |
| | PRDWQRLINNNWGFRPKRLNFKLFNIQVKEVTDNNGVKTIANNLTSTVQVF | |
| | TDSDYQLPYVLGSAHEGCLPPFPADVFMIPQYGYLTLNDGSQAVGRSSFY | |
| | CLEYFPSQMLRTGNNFQFSYEFENVPFHSSYAHSQSLDRLMNPLIDQYLY | |
| | YLSKTINGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGSLIFG | |
| | KQGTGRDNVDADKVMITNEEEIKTTNPVATEAYGTVATNKQSAYAQAQTG | |
| | AVQNQGILPGMVWQDRDVYLQGPIWAKIPHTDGNFHPSPLMGGFGMKHP | |
| | PPQILIKNTPVPADPPTAFNKDKLNSFITQYSTGQVSVEIEWELQKENSKRW | |
| | NPEIQYTSNYYKSNNVEFAVNTEGVYSEPRPIGTRYLTRNL | |
| V82 (amino | MAADGYLPDWLEDNLSEGIREWWALKPGAPQPKANQQHQDNARGLVLP | 377 |
| acid) | GYKYLGPGNGLDKGEPVNAADAAALEHDKAYDQQLKAGDNPYLKYNHAD | |
| | AEFQERLKEDTSFGGNLGRAVFQAKKRLLEPLGLVEEAAKTAPGKKRPVE | |
| | QSPQEPDSSAGIGKSGAQPAKKRLNFGQTGDTESVPDPQPIGEPPAAPSG | |
| | VGSLTMASGGGAPVADNNEGADGVGSSSGNWHCDSQWLGDRVITTSTR | |
| | TWALPTYNNHLYKQISNSTSGGSSNDNAYFGYSTPWGYFDFNRFHCHFS | |
| | PRDWQRLINNNWGFRPKRLNFKLFNIQVKEVTDNNGVKTIANNLTSTVQVF | |
| | TDSDYQLPYVLGSAHEGCLPPFPADVFMIPQYGYLTLNDGSQAVGRSSFY | |
| | CLEYFPSQMLRTGNNFQFSYEFENVPFHSSYAHSQSLDRLMNPLIDQYLY | |
| | YLSKTINGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGSLIFG | |
| | KQGTGRDNVDADKVMITNEEEIKTTNPVATESYGTVATNHQSAYAQAQTG | |
| | ALQSQGILPGMVWQDRDVYLQGPIWAKIPHTDGNFHPSPLMGGFGMKHP | |
| | PPQILIKNTPVPADPPTAFNKDKLNSFITQYSTGQVSVEIEWELQKENSKRW | |
| | NPEIQYTSNYYKSNNVEFAVNTEGVYSEPRPIGTRYLTRNL | |
| V83 (amino | MAADGYLPDWLEDNLSEGIREWWALKPGAPQPKANQQHQDNARGLVLP | 378 |
| acid) | GYKYLGPGNGLDKGEPVNAADAAALEHDKAYDQQLKAGDNPYLKYNHAD | |
| | AEFQERLKEDTSFGGNLGRAVFQAKKRLLEPLGLVEEAAKTAPGKKRPVE | |
| | QSPQEPDSSAGIGKSGAQPAKKRLNFGQTGDTESVPDPQPIGEPPAAPSG | |
| | VGSLTMASGGGAPVADNNEGADGVGSSSGNWHCDSQWLGDRVITTSTR | |
| | TWALPTYNNHLYKQISNSTSGGSSNDNAYFGYSTPWGYFDFNRFHCHFS | |
| | PRDWQRLINNNWGFRPKRLNFKLFNIQVKEVTDNNGVKTIANNLTSTVQVF | |
| | TDSDYQLPYVLGSAHEGCLPPFPADVFMIPQYGYLTLNDGSQAVGRSSFY | |
| | CLEYFPSQMLRTGNNFQFSYEFENVPFHSSYAHSQSLDRLMNPLIDQYLY | |
| | YLSKTINGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGSLIFG | |
| | KQGTGHDNVDADKVMITNEEEIKTTNPVATESYGQVATNHQSAYAQAQTG | |
| | ALQSQGILPGMVWQDRDVYLQGPIWAKIPHTDGNFHPSPLMGGFGMKHP | |
| | PPQILIKNTPVPADPPTAFNKDKLNSFITQYSTGQVSVEIEWELQKENSKRW | |
| | NPEIQYTSNYYKSNNVEFAVNTEGVYSEPRPIGTRYLTRNL | |
| V84 (amino | MAADGYLPDWLEDNLSEGIREWWALKPGAPQPKANQQHQDNARGLVLP | 379 |
| acid) | GYKYLGPGNGLDKGEPVNAADAAALEHDKAYDQQLKAGDNPYLKYNHAD | |
| | AEFQERLKEDTSFGGNLGRAVFQAKKRLLEPLGLVEEAAKTAPGKKRPVE | |
| | QSPQEPDSSAGIGKSGAQPAKKRLNFGQTGDTESVPDPQPIGEPPAAPSG | |
| | VGSLTMASGGGAPVADNNEGADGVGSSSGNWHCDSQWLGDRVITTSTR | |
| | TWALPTYNNHLYKQISNSTSGGSSNDNAYFGYSTPWGYFDFNRFHCHFS | |
| | PRDWQRLINNNWGFRPKRLNFKLFNIQVKEVTDNNGVKTIANNLTSTVQVF | |
| | TDSDYQLPYVLGSAHEGCLPPFPADVFMIPQYGYLTLNDGSQAVGRSSFY | |
| | CLEYFPSQMLRTGNNFQFSYEFENVPFHSSYAHSQSLDRLMNPLIDQYLY | |
| | YLSKTINGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGSLIFG | |
| | KQGTGRDNVDADKVMITNEEEIKTTNPVATESYGTVATNKQSAQAQAQTG | |
| | WLQSQGVLPGMVWQDRDVYLQGPIWAKIPHTDGNFHPSPLMGGFGMKH | |
| | PPPQILIKNTPVPADPPTAFNKDKLNSFITQYSTGQVSVEIEWELQKENSKR | |
| | WNPEIQYTSNYYKSNNVEFAVNTEGVYSEPRPIGTRYLTRNL | |
| V85 (amino | MAADGYLPDWLEDNLSEGIREWWALKPGAPQPKANQQHQDNARGLVLP | 380 |
| acid) | GYKYLGPGNGLDKGEPVNAADAAALEHDKAYDQQLKAGDNPYLKYNHAD | |
| | AEFQERLKEDTSFGGNLGRAVFQAKKRLLEPLGLVEEAAKTAPGKKRPVE | |
| | QSPQEPDSSAGIGKSGAQPAKKRLNFGQTGDTESVPDPQPIGEPPAAPSG | |
| | VGSLTMASGGGAPVADNNEGADGVGSSSGNWHCDSQWLGDRVITTSTR | |
| | TWALPTYNNHLYKQISNSTSGGSSNDNAYFGYSTPWGYFDFNRFHCHFS | |
| | PRDWQRLINNNWGFRPKRLNFKLFNIQVKEVTDNNGVKTIANNLTSTVQVF | |
| | TDSDYQLPYVLGSAHEGCLPPFPADVFMIPQYGYLTLNDGSQAVGRSSFY | |
| | CLEYFPSQMLRTGNNFQFSYEFENVPFHSSYAHSQSLDRLMNPLIDQYLY | |
| | YLSKTINGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGSLIFG | |
| | KQGTGHDNVDADKVMITNEEEIKTTNPVATEAYGTVATNHQSAYAQAQTG | |
| | AVQNQGILPGMVWQDRDVYLQGPIWAKIPHTDGNFHPSPLMGGFGMKHP | |
| | PPQILIKNTPVPADPPTAFNKDKLNSFITQYSTGQVSVEIEWELQKENSKRW | |
| | NPEIQYTSNYYKSNNVEFAVNTEGVYSEPRPIGTRYLTRNL | |
| V86 (amino | MAADGYLPDWLEDNLSEGIREWWALKPGAPQPKANQQHQDNARGLVLP | 381 |
| acid) | GYKYLGPGNGLDKGEPVNAADAAALEHDKAYDQQLKAGDNPYLKYNHAD | |
| | AEFQERLKEDTSFGGNLGRAVFQAKKRLLEPLGLVEEAAKTAPGKKRPVE | |
| | QSPQEPDSSAGIGKSGAQPAKKRLNFGQTGDTESVPDPQPIGEPPAAPSG | |
| | VGSLTMASGGGAPVADNNEGADGVGSSSGNWHCDSQWLGDRVITTSTR | |
| | TWALPTYNNHLYKQISNSTSGGSSNDNAYFGYSTPWGYFDFNRFHCHFS | |
| | PRDWQRLINNNWGFRPKRLNFKLFNIQVKEVTDNNGVKTIANNLTSTVQVF | |
| | TDSDYQLPYVLGSAHEGCLPPFPADVFMIPQYGYLTLNDGSQAVGRSSFY | |
| | CLEYFPSQMLRTGNNFQFSYEFENVPFHSSYAHSQSLDRLMNPLIDQYLY | |
| | YLSKTINGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGSLIFG | |
| | KQGTGHDNVDADKVMITNEEEIKTTNPVATESYGTVATNKQSAQAQAQTG | |
| | WVQSQGVLPGMVWQDRDVYLQGPIWAKIPHTDGNFHPSPLMGGFGMKH | |
| | PPPQILIKNTPVPADPPTAFNKDKLNSFITQYSTGQVSVEIEWELQKENSKR | |
| | WNPEIQYTSNYYKSNNVEFAVNTEGVYSEPRPIGTRYLTRNL | |
| V87 (amino | MAADGYLPDWLEDNLSEGIREWWALKPGAPQPKANQQHQDNARGLVLP | 382 |
| acid) | GYKYLGPGNGLDKGEPVNAADAAALEHDKAYDQQLKAGDNPYLKYNHAD | |
| | AEFQERLKEDTSFGGNLGRAVFQAKKRLLEPLGLVEEAAKTAPGKKRPVE | |
| | QSPQEPDSSAGIGKSGAQPAKKRLNFGQTGDTESVPDPQPIGEPPAAPSG | |
| | VGSLTMASGGGAPVADNNEGADGVGSSSGNWHCDSQWLGDRVITTSTR | |
| | TWALPTYNNHLYKQISNSTSGGSSNDNAYFGYSTPWGYFDFNRFHCHFS | |
| | PRDWQRLINNNWGFRPKRLNFKLFNIQVKEVTDNNGVKTIANNLTSTVQVF | |
| | TDSDYQLPYVLGSAHEGCLPPFPADVFMIPQYGYLTLNDGSQAVGRSSFY | |
| | CLEYFPSQMLRTGNNFQFSYEFENVPFHSSYAHSQSLDRLMNPLIDQYLY | |
| | YLSKTINGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGSLIFG | |
| | KQGTGHDNVDADKVMITNEEEIKTTNPVATESYGQVATNHQSAYAQAQTG | |
| | AVQSQGVLPGMVWQDRDVYLQGPIWAKIPHTDGNFHPSPLMGGFGMKH | |
| | PPPQILIKNTPVPADPPTAFNKDKLNSFITQYSTGQVSVEIEWELQKENSKR | |
| | WNPEIQYTSNYYKSNNVEFAVNTEGVYSEPRPIGTRYLTRNL | |
| V88 (amino | MAADGYLPDWLEDNLSEGIREWWALKPGAPQPKANQQHQDNARGLVLP | 383 |
| acid) | GYKYLGPGNGLDKGEPVNAADAAALEHDKAYDQQLKAGDNPYLKYNHAD | |
| | AEFQERLKEDTSFGGNLGRAVFQAKKRLLEPLGLVEEAAKTAPGKKRPVE | |
| | QSPQEPDSSAGIGKSGAQPAKKRLNFGQTGDTESVPDPQPIGEPPAAPSG | |
| | VGSLTMASGGGAPVADNNEGADGVGSSSGNWHCDSQWLGDRVITTSTR | |
| | TWALPTYNNHLYKQISNSTSGGSSNDNAYFGYSTPWGYFDFNRFHCHFS | |
| | PRDWQRLINNNWGFRPKRLNFKLFNIQVKEVTDNNGVKTIANNLTSTVQVF | |
| | TDSDYQLPYVLGSAHEGCLPPFPADVFMIPQYGYLTLNDGSQAVGRSSFY | |
| | CLEYFPSQMLRTGNNFQFSYEFENVPFHSSYAHSQSLDRLMNPLIDQYLY | |
| | YLSKTINGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGSLIFG | |
| | KQGTGRDNVDADKVMITNEEEIKTTNPVATESYGTVATNKQSAYAQAQTG | |
| | ALQNQGILPGMVWQDRDVYLQGPIWAKIPHTDGNFHPSPLMGGFGMKHP | |
| | PPQILIKNTPVPADPPTAFNKDKLNSFITQYSTGQVSVEIEWELQKENSKRW | |
| | NPEIQYTSNYYKSNNVEFAVNTEGVYSEPRPIGTRYLTRNL | |
| V89 (amino | MAADGYLPDWLEDNLSEGIREWWALKPGAPQPKANQQHQDNARGLVLP | 384 |
| acid) | GYKYLGPGNGLDKGEPVNAADAAALEHDKAYDQQLKAGDNPYLKYNHAD | |
| | AEFQERLKEDTSFGGNLGRAVFQAKKRLLEPLGLVEEAAKTAPGKKRPVE | |
| | QSPQEPDSSAGIGKSGAQPAKKRLNFGQTGDTESVPDPQPIGEPPAAPSG | |
| | VGSLTMASGGGAPVADNNEGADGVGSSSGNWHCDSQWLGDRVITTSTR | |
| | TWALPTYNNHLYKQISNSTSGGSSNDNAYFGYSTPWGYFDFNRFHCHFS | |
| | PRDWQRLINNNWGFRPKRLNFKLFNIQVKEVTDNNGVKTIANNLTSTVQVF | |
| | TDSDYQLPYVLGSAHEGCLPPFPADVFMIPQYGYLTLNDGSQAVGRSSFY | |
| | CLEYFPSQMLRTGNNFQFSYEFENVPFHSSYAHSQSLDRLMNPLIDQYLY | |
| | YLSKTINGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGSLIFG | |
| | KQGTGHDNVDADKVMITNEEEIKTTNPVATEAYGQVATNHQSAYAQAQTG | |
| | ALQNQGILPGMVWQDRDVYLQGPIWAKIPHTDGNFHPSPLMGGFGMKHP | |
| | PPQILIKNTPVPADPPTAFNKDKLNSFITQYSTGQVSVEIEWELQKENSKRW | |
| | NPEIQYTSNYYKSNNVEFAVNTEGVYSEPRPIGTRYLTRNL | |
| V90 (amino | MAADGYLPDWLEDNLSEGIREWWALKPGAPQPKANQQHQDNARGLVLP | 385 |
| acid) | GYKYLGPGNGLDKGEPVNAADAAALEHDKAYDQQLKAGDNPYLKYNHAD | |
| | AEFQERLKEDTSFGGNLGRAVFQAKKRLLEPLGLVEEAAKTAPGKKRPVE | |
| | QSPQEPDSSAGIGKSGAQPAKKRLNFGQTGDTESVPDPQPIGEPPAAPSG | |
| | VGSLTMASGGGAPVADNNEGADGVGSSSGNWHCDSQWLGDRVITTSTR | |
| | TWALPTYNNHLYKQISNSTSGGSSNDNAYFGYSTPWGYFDFNRFHCHFS | |
| | PRDWQRLINNNWGFRPKRLNFKLFNIQVKEVTDNNGVKTIANNLTSTVQVF | |
| | TDSDYQLPYVLGSAHEGCLPPFPADVFMIPQYGYLTLNDGSQAVGRSSFY | |
| | CLEYFPSQMLRTGNNFQFSYEFENVPFHSSYAHSQSLDRLMNPLIDQYLY | |
| | YLSKTINGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGSLIFG | |
| | KQGTGHDNVDADKVMITNEEEIKTTNPVATEAYGQVATNKQSAYAQAQTG | |
| | AVQNQGILPGMVWQDRDVYLQGPIWAKIPHTDGNFHPSPLMGGFGMKHP | |
| | PPQILIKNTPVPADPPTAFNKDKLNSFITQYSTGQVSVEIEWELQKENSKRW | |
| | NPEIQYTSNYYKSNNVEFAVNTEGVYSEPRPIGTRYLTRNL | |
| V91 (amino | MAADGYLPDWLEDNLSEGIREWWALKPGAPQPKANQQHQDNARGLVLP | 386 |
| acid) | GYKYLGPGNGLDKGEPVNAADAAALEHDKAYDQQLKAGDNPYLKYNHAD | |
| | AEFQERLKEDTSFGGNLGRAVFQAKKRLLEPLGLVEEAAKTAPGKKRPVE | |
| | QSPQEPDSSAGIGKSGAQPAKKRLNFGQTGDTESVPDPQPIGEPPAAPSG | |
| | VGSLTMASGGGAPVADNNEGADGVGSSSGNWHCDSQWLGDRVITTSTR | |
| | TWALPTYNNHLYKQISNSTSGGSSNDNAYFGYSTPWGYFDFNRFHCHFS | |
| | PRDWQRLINNNWGFRPKRLNFKLFNIQVKEVTDNNGVKTIANNLTSTVQVF | |
| | TDSDYQLPYVLGSAHEGCLPPFPADVFMIPQYGYLTLNDGSQAVGRSSFY | |
| | CLEYFPSQMLRTGNNFQFSYEFENVPFHSSYAHSQSLDRLMNPLIDQYLY | |
| | YLSKTINGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGSLIFG | |
| | KQGTGRDNVDADKVMITNEEEIKTTNPVATEAYGTVATNHQSAYAQAQTG | |
| | AVQNQGVLPGMVWQDRDVYLQGPIWAKIPHTDGNFHPSPLMGGFGMKH | |
| | PPPQILIKNTPVPADPPTAFNKDKLNSFITQYSTGQVSVEIEWELQKENSKR | |
| | WNPEIQYTSNYYKSNNVEFAVNTEGVYSEPRPIGTRYLTRNL | |
| V92 (amino | MAADGYLPDWLEDNLSEGIREWWALKPGAPQPKANQQHQDNARGLVLP | 387 |
| acid) | GYKYLGPGNGLDKGEPVNAADAAALEHDKAYDQQLKAGDNPYLKYNHAD | |
| | AEFQERLKEDTSFGGNLGRAVFQAKKRLLEPLGLVEEAAKTAPGKKRPVE | |
| | QSPQEPDSSAGIGKSGAQPAKKRLNFGQTGDTESVPDPQPIGEPPAAPSG | |
| | VGSLTMASGGGAPVADNNEGADGVGSSSGNWHCDSQWLGDRVITTSTR | |
| | TWALPTYNNHLYKQISNSTSGGSSNDNAYFGYSTPWGYFDFNRFHCHFS | |
| | PRDWQRLINNNWGFRPKRLNFKLFNIQVKEVTDNNGVKTIANNLTSTVQVF | |
| | TDSDYQLPYVLGSAHEGCLPPFPADVFMIPQYGYLTLNDGSQAVGRSSFY | |
| | CLEYFPSQMLRTGNNFQFSYEFENVPFHSSYAHSQSLDRLMNPLIDQYLY | |
| | YLSKTINGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGSLIFG | |
| | KQGTGRDNVDADKVMITNEEEIKTTNPVATESYGTVATNHQSAQAQAQTG | |
| | ALQSQGVLPGMVWQDRDVYLQGPIWAKIPHTDGNFHPSPLMGGFGMKHP | |
| | PPQILIKNTPVPADPPTAFNKDKLNSFITQYSTGQVSVEIEWELQKENSKRW | |
| | NPEIQYTSNYYKSNNVEFAVNTEGVYSEPRPIGTRYLTRNL | |
| V93 (amino | MAADGYLPDWLEDNLSEGIREWWALKPGAPQPKANQQHQDNARGLVLP | 388 |
| acid) | GYKYLGPGNGLDKGEPVNAADAAALEHDKAYDQQLKAGDNPYLKYNHAD | |
| | AEFQERLKEDTSFGGNLGRAVFQAKKRLLEPLGLVEEAAKTAPGKKRPVE | |
| | QSPQEPDSSAGIGKSGAQPAKKRLNFGQTGDTESVPDPQPIGEPPAAPSG | |
| | VGSLTMASGGGAPVADNNEGADGVGSSSGNWHCDSQWLGDRVITTSTR | |
| | TWALPTYNNHLYKQISNSTSGGSSNDNAYFGYSTPWGYFDFNRFHCHFS | |
| | PRDWQRLINNNWGFRPKRLNFKLFNIQVKEVTDNNGVKTIANNLTSTVQVF | |
| | TDSDYQLPYVLGSAHEGCLPPFPADVFMIPQYGYLTLNDGSQAVGRSSFY | |
| | CLEYFPSQMLRTGNNFQFSYEFENVPFHSSYAHSQSLDRLMNPLIDQYLY | |
| | YLSKTINGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGSLIFG | |
| | KQGTGHDNVDADKVMITNEEEIKTTNPVATESYGQVATNKQSAYAQAQTG | |
| | AVQNQGVLPGMVWQDRDVYLQGPIWAKIPHTDGNFHPSPLMGGFGMKH | |
| | PPPQILIKNTPVPADPPTAFNKDKLNSFITQYSTGQVSVEIEWELQKENSKR | |
| | WNPEIQYTSNYYKSNNVEFAVNTEGVYSEPRPIGTRYLTRNL | |
| V94 (amino | MAADGYLPDWLEDNLSEGIREWWALKPGAPQPKANQQHQDNARGLVLP | 389 |
| acid) | GYKYLGPGNGLDKGEPVNAADAAALEHDKAYDQQLKAGDNPYLKYNHAD | |
| | AEFQERLKEDTSFGGNLGRAVFQAKKRLLEPLGLVEEAAKTAPGKKRPVE | |
| | QSPQEPDSSAGIGKSGAQPAKKRLNFGQTGDTESVPDPQPIGEPPAAPSG | |
| | VGSLTMASGGGAPVADNNEGADGVGSSSGNWHCDSQWLGDRVITTSTR | |
| | TWALPTYNNHLYKQISNSTSGGSSNDNAYFGYSTPWGYFDFNRFHCHFS | |
| | PRDWQRLINNNWGFRPKRLNFKLFNIQVKEVTDNNGVKTIANNLTSTVQVF | |
| | TDSDYQLPYVLGSAHEGCLPPFPADVFMIPQYGYLTLNDGSQAVGRSSFY | |
| | CLEYFPSQMLRTGNNFQFSYEFENVPFHSSYAHSQSLDRLMNPLIDQYLY | |
| | YLSKTINGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGSLIFG | |
| | KQGTGHDNVDADKVMITNEEEIKTTNPVATESYGQVATNHQSAYAQAQTG | |
| | ALQNQGVLPGMVWQDRDVYLQGPIWAKIPHTDGNFHPSPLMGGFGMKH | |
| | PPPQILIKNTPVPADPPTAFNKDKLNSFITQYSTGQVSVEIEWELQKENSKR | |
| | WNPEIQYTSNYYKSNNVEFAVNTEGVYSEPRPIGTRYLTRNL | |
| V95 (amino | MAADGYLPDWLEDNLSEGIREWWALKPGAPQPKANQQHQDNARGLVLP | 390 |
| acid) | GYKYLGPGNGLDKGEPVNAADAAALEHDKAYDQQLKAGDNPYLKYNHAD | |
| | AEFQERLKEDTSFGGNLGRAVFQAKKRLLEPLGLVEEAAKTAPGKKRPVE | |
| | QSPQEPDSSAGIGKSGAQPAKKRLNFGQTGDTESVPDPQPIGEPPAAPSG | |
| | VGSLTMASGGGAPVADNNEGADGVGSSSGNWHCDSQWLGDRVITTSTR | |
| | TWALPTYNNHLYKQISNSTSGGSSNDNAYFGYSTPWGYFDFNRFHCHFS | |
| | PRDWQRLINNNWGFRPKRLNFKLFNIQVKEVTDNNGVKTIANNLTSTVQVF | |
| | TDSDYQLPYVLGSAHEGCLPPFPADVFMIPQYGYLTLNDGSQAVGRSSFY | |
| | CLEYFPSQMLRTGNNFQFSYEFENVPFHSSYAHSQSLDRLMNPLIDQYLY | |
| | YLSKTINGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGSLIFG | |
| | KQGTGRDNVDADKVMITNEEEIKTTNPVATEAYGQVATNHQSAYAQAQTG | |
| | ALQNQGVLPGMVWQDRDVYLQGPIWAKIPHTDGNFHPSPLMGGFGMKH | |
| | PPPQILIKNTPVPADPPTAFNKDKLNSFITQYSTGQVSVEIEWELQKENSKR | |
| | WNPEIQYTSNYYKSNNVEFAVNTEGVYSEPRPIGTRYLTRNL | |
| V96 (amino | MAADGYLPDWLEDNLSEGIREWWALKPGAPQPKANQQHQDNARGLVLP | 391 |
| acid) | GYKYLGPGNGLDKGEPVNAADAAALEHDKAYDQQLKAGDNPYLKYNHAD | |
| | AEFQERLKEDTSFGGNLGRAVFQAKKRLLEPLGLVEEAAKTAPGKKRPVE | |
| | QSPQEPDSSAGIGKSGAQPAKKRLNFGQTGDTESVPDPQPIGEPPAAPSG | |
| | VGSLTMASGGGAPVADNNEGADGVGSSSGNWHCDSQWLGDRVITTSTR | |
| | TWALPTYNNHLYKQISNSTSGGSSNDNAYFGYSTPWGYFDFNRFHCHFS | |
| | PRDWQRLINNNWGFRPKRLNFKLFNIQVKEVTDNNGVKTIANNLTSTVQVF | |
| | TDSDYQLPYVLGSAHEGCLPPFPADVFMIPQYGYLTLNDGSQAVGRSSFY | |
| | CLEYFPSQMLRTGNNFQFSYEFENVPFHSSYAHSQSLDRLMNPLIDQYLY | |
| | YLSKTINGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGSLIFG | |
| | KQGTGHDNVDADKVMITNEEEIKTTNPVATEAYGQVATNHQSAYAQAQTG | |
| | AVQSQGILPGMVWQDRDVYLQGPIWAKIPHTDGNFHPSPLMGGFGMKHP | |
| | PPQILIKNTPVPADPPTAFNKDKLNSFITQYSTGQVSVEIEWELQKENSKRW | |
| | NPEIQYTSNYYKSNNVEFAVNTEGVYSEPRPIGTRYLTRNL | |
| V97 (amino | MAADGYLPDWLEDNLSEGIREWWALKPGAPQPKANQQHQDNARGLVLP | 392 |
| acid) | GYKYLGPGNGLDKGEPVNAADAAALEHDKAYDQQLKAGDNPYLKYNHAD | |
| | AEFQERLKEDTSFGGNLGRAVFQAKKRLLEPLGLVEEAAKTAPGKKRPVE | |
| | QSPQEPDSSAGIGKSGAQPAKKRLNFGQTGDTESVPDPQPIGEPPAAPSG | |
| | VGSLTMASGGGAPVADNNEGADGVGSSSGNWHCDSQWLGDRVITTSTR | |
| | TWALPTYNNHLYKQISNSTSGGSSNDNAYFGYSTPWGYFDFNRFHCHFS | |
| | PRDWQRLINNNWGFRPKRLNFKLFNIQVKEVTDNNGVKTIANNLTSTVQVF | |
| | TDSDYQLPYVLGSAHEGCLPPFPADVFMIPQYGYLTLNDGSQAVGRSSFY | |
| | CLEYFPSQMLRTGNNFQFSYEFENVPFHSSYAHSQSLDRLMNPLIDQYLY | |
| | YLSKTINGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGSLIFG | |
| | KQGTGRDNVDADKVMITNEEEIKTTNPVATESYGTVATNHQSAYAQAQTG | |
| | AVQSQGVLPGMVWQDRDVYLQGPIWAKIPHTDGNFHPSPLMGGFGMKH | |
| | PPPQILIKNTPVPADPPTAFNKDKLNSFITQYSTGQVSVEIEWELQKENSKR | |
| | WNPEIQYTSNYYKSNNVEFAVNTEGVYSEPRPIGTRYLTRNL | |
| V98 (amino | MAADGYLPDWLEDNLSEGIREWWALKPGAPQPKANQQHQDNARGLVLP | 393 |
| acid) | GYKYLGPGNGLDKGEPVNAADAAALEHDKAYDQQLKAGDNPYLKYNHAD | |
| | AEFQERLKEDTSFGGNLGRAVFQAKKRLLEPLGLVEEAAKTAPGKKRPVE | |
| | QSPQEPDSSAGIGKSGAQPAKKRLNFGQTGDTESVPDPQPIGEPPAAPSG | |
| | VGSLTMASGGGAPVADNNEGADGVGSSSGNWHCDSQWLGDRVITTSTR | |
| | TWALPTYNNHLYKQISNSTSGGSSNDNAYFGYSTPWGYFDFNRFHCHFS | |
| | PRDWQRLINNNWGFRPKRLNFKLFNIQVKEVTDNNGVKTIANNLTSTVQVF | |
| | TDSDYQLPYVLGSAHEGCLPPFPADVFMIPQYGYLTLNDGSQAVGRSSFY | |
| | CLEYFPSQMLRTGNNFQFSYEFENVPFHSSYAHSQSLDRLMNPLIDQYLY | |
| | YLSKTINGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGSLIFG | |
| | KQGTGHDNVDADKVMITNEEEIKTTNPVATEAYGQVATNHQSAYAQAQTG | |
| | AVQNQGVLPGMVWQDRDVYLQGPIWAKIPHTDGNFHPSPLMGGFGMKH | |
| | PPPQILIKNTPVPADPPTAFNKDKLNSFITQYSTGQVSVEIEWELQKENSKR | |
| | WNPEIQYTSNYYKSNNVEFAVNTEGVYSEPRPIGTRYLTRNL | |
| V99 (amino | MAADGYLPDWLEDNLSEGIREWWALKPGAPQPKANQQHQDNARGLVLP | 394 |
| acid) | GYKYLGPGNGLDKGEPVNAADAAALEHDKAYDQQLKAGDNPYLKYNHAD | |
| | AEFQERLKEDTSFGGNLGRAVFQAKKRLLEPLGLVEEAAKTAPGKKRPVE | |
| | QSPQEPDSSAGIGKSGAQPAKKRLNFGQTGDTESVPDPQPIGEPPAAPSG | |
| | VGSLTMASGGGAPVADNNEGADGVGSSSGNWHCDSQWLGDRVITTSTR | |
| | TWALPTYNNHLYKQISNSTSGGSSNDNAYFGYSTPWGYFDFNRFHCHFS | |
| | PRDWQRLINNNWGFRPKRLNFKLFNIQVKEVTDNNGVKTIANNLTSTVQVF | |
| | TDSDYQLPYVLGSAHEGCLPPFPADVFMIPQYGYLTLNDGSQAVGRSSFY | |
| | CLEYFPSQMLRTGNNFQFSYEFENVPFHSSYAHSQSLDRLMNPLIDQYLY | |
| | YLSKTINGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGSLIFG | |
| | KQGTGRDNVDADKVMITNEEEIKTTNPVATEAYGQVATNHQSAYAQAQTG | |
| | ALQSQGILPGMVWQDRDVYLQGPIWAKIPHTDGNFHPSPLMGGFGMKHP | |
| | PPQILIKNTPVPADPPTAFNKDKLNSFITQYSTGQVSVEIEWELQKENSKRW | |
| | NPEIQYTSNYYKSNNVEFAVNTEGVYSEPRPIGTRYLTRNL | |
| V100 (amino | MAADGYLPDWLEDNLSEGIREWWALKPGAPQPKANQQHQDNARGLVLP | 395 |
| acid) | GYKYLGPGNGLDKGEPVNAADAAALEHDKAYDQQLKAGDNPYLKYNHAD | |
| | AEFQERLKEDTSFGGNLGRAVFQAKKRLLEPLGLVEEAAKTAPGKKRPVE | |
| | QSPQEPDSSAGIGKSGAQPAKKRLNFGQTGDTESVPDPQPIGEPPAAPSG | |
| | VGSLTMASGGGAPVADNNEGADGVGSSSGNWHCDSQWLGDRVITTSTR | |
| | TWALPTYNNHLYKQISNSTSGGSSNDNAYFGYSTPWGYFDFNRFHCHFS | |
| | PRDWQRLINNNWGFRPKRLNFKLFNIQVKEVTDNNGVKTIANNLTSTVQVF | |
| | TDSDYQLPYVLGSAHEGCLPPFPADVFMIPQYGYLTLNDGSQAVGRSSFY | |
| | CLEYFPSQMLRTGNNFQFSYEFENVPFHSSYAHSQSLDRLMNPLIDQYLY | |
| | YLSKTINGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGSLIFG | |
| | KQGTGHDNVDADKVMITNEEEIKTTNPVATESYGQVATNKQSAYAQAQTG | |
| | ALQNQGILPGMVWQDRDVYLQGPIWAKIPHTDGNFHPSPLMGGFGMKHP | |
| | PPQILIKNTPVPADPPTAFNKDKLNSFITQYSTGQVSVEIEWELQKENSKRW | |
| | NPEIQYTSNYYKSNNVEFAVNTEGVYSEPRPIGTRYLTRNL | |
| V101 (amino | MAADGYLPDWLEDNLSEGIREWWALKPGAPQPKANQQHQDNARGLVLP | 396 |
| acid) | GYKYLGPGNGLDKGEPVNAADAAALEHDKAYDQQLKAGDNPYLKYNHAD | |
| | AEFQERLKEDTSFGGNLGRAVFQAKKRLLEPLGLVEEAAKTAPGKKRPVE | |
| | QSPQEPDSSAGIGKSGAQPAKKRLNFGQTGDTESVPDPQPIGEPPAAPSG | |
| | VGSLTMASGGGAPVADNNEGADGVGSSSGNWHCDSQWLGDRVITTSTR | |
| | TWALPTYNNHLYKQISNSTSGGSSNDNAYFGYSTPWGYFDFNRFHCHFS | |
| | PRDWQRLINNNWGFRPKRLNFKLFNIQVKEVTDNNGVKTIANNLTSTVQVF | |
| | TDSDYQLPYVLGSAHEGCLPPFPADVFMIPQYGYLTLNDGSQAVGRSSFY | |
| | CLEYFPSQMLRTGNNFQFSYEFENVPFHSSYAHSQSLDRLMNPLIDQYLY | |
| | YLSKTINGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGSLIFG | |
| | KQGTGRDNVDADKVMITNEEEIKTTNPVATESYGTVATNHQSAYAQAQTG | |
| | ALQNQGVLPGMVWQDRDVYLQGPIWAKIPHTDGNFHPSPLMGGFGMKH | |
| | PPPQILIKNTPVPADPPTAFNKDKLNSFITQYSTGQVSVEIEWELQKENSKR | |
| | WNPEIQYTSNYYKSNNVEFAVNTEGVYSEPRPIGTRYLTRNL | |
| V102 (amino | MAADGYLPDWLEDNLSEGIREWWALKPGAPQPKANQQHQDNARGLVLP | 397 |
| acid) | GYKYLGPGNGLDKGEPVNAADAAALEHDKAYDQQLKAGDNPYLKYNHAD | |
| | AEFQERLKEDTSFGGNLGRAVFQAKKRLLEPLGLVEEAAKTAPGKKRPVE | |
| | QSPQEPDSSAGIGKSGAQPAKKRLNFGQTGDTESVPDPQPIGEPPAAPSG | |
| | VGSLTMASGGGAPVADNNEGADGVGSSSGNWHCDSQWLGDRVITTSTR | |
| | TWALPTYNNHLYKQISNSTSGGSSNDNAYFGYSTPWGYFDFNRFHCHFS | |
| | PRDWQRLINNNWGFRPKRLNFKLFNIQVKEVTDNNGVKTIANNLTSTVQVF | |
| | TDSDYQLPYVLGSAHEGCLPPFPADVFMIPQYGYLTLNDGSQAVGRSSFY | |
| | CLEYFPSQMLRTGNNFQFSYEFENVPFHSSYAHSQSLDRLMNPLIDQYLY | |
| | YLSKTINGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGSLIFG | |
| | KQGTGRDNVDADKVMITNEEEIKTTNPVATEAYGQVATNHQSAYAQAQTG | |
| | AVQSQGVLPGMVWQDRDVYLQGPIWAKIPHTDGNFHPSPLMGGFGMKH | |
| | PPPQILIKNTPVPADPPTAFNKDKLNSFITQYSTGQVSVEIEWELQKENSKR | |
| | WNPEIQYTSNYYKSNNVEFAVNTEGVYSEPRPIGTRYLTRNL | |
| V103 (amino | MAADGYLPDWLEDNLSEGIREWWALKPGAPQPKANQQHQDNARGLVLP | 398 |
| acid) | GYKYLGPGNGLDKGEPVNAADAAALEHDKAYDQQLKAGDNPYLKYNHAD | |
| | AEFQERLKEDTSFGGNLGRAVFQAKKRLLEPLGLVEEAAKTAPGKKRPVE | |
| | QSPQEPDSSAGIGKSGAQPAKKRLNFGQTGDTESVPDPQPIGEPPAAPSG | |
| | VGSLTMASGGGAPVADNNEGADGVGSSSGNWHCDSQWLGDRVITTSTR | |
| | TWALPTYNNHLYKQISNSTSGGSSNDNAYFGYSTPWGYFDFNRFHCHFS | |
| | PRDWQRLINNNWGFRPKRLNFKLFNIQVKEVTDNNGVKTIANNLTSTVQVF | |
| | TDSDYQLPYVLGSAHEGCLPPFPADVFMIPQYGYLTLNDGSQAVGRSSFY | |
| | CLEYFPSQMLRTGNNFQFSYEFENVPFHSSYAHSQSLDRLMNPLIDQYLY | |
| | YLSKTINGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGSLIFG | |
| | KQGTGHDNVDADKVMITNEEEIKTTNPVATESYGTVATNHQSAYAQAQTG | |
| | ALQNQGILPGMVWQDRDVYLQGPIWAKIPHTDGNFHPSPLMGGFGMKHP | |
| | PPQILIKNTPVPADPPTAFNKDKLNSFITQYSTGQVSVEIEWELQKENSKRW | |
| | NPEIQYTSNYYKSNNVEFAVNTEGVYSEPRPIGTRYLTRNL | |
| V104 (amino | MAADGYLPDWLEDNLSEGIREWWALKPGAPQPKANQQHQDNARGLVLP | 399 |
| acid) | GYKYLGPGNGLDKGEPVNAADAAALEHDKAYDQQLKAGDNPYLKYNHAD | |
| | AEFQERLKEDTSFGGNLGRAVFQAKKRLLEPLGLVEEAAKTAPGKKRPVE | |
| | QSPQEPDSSAGIGKSGAQPAKKRLNFGQTGDTESVPDPQPIGEPPAAPSG | |
| | VGSLTMASGGGAPVADNNEGADGVGSSSGNWHCDSQWLGDRVITTSTR | |
| | TWALPTYNNHLYKQISNSTSGGSSNDNAYFGYSTPWGYFDFNRFHCHFS | |
| | PRDWQRLINNNWGFRPKRLNFKLFNIQVKEVTDNNGVKTIANNLTSTVQVF | |
| | TDSDYQLPYVLGSAHEGCLPPFPADVFMIPQYGYLTLNDGSQAVGRSSFY | |
| | CLEYFPSQMLRTGNNFQFSYEFENVPFHSSYAHSQSLDRLMNPLIDQYLY | |
| | YLSKTINGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGSLIFG | |
| | KQGTGRDNVDADKVMITNEEEIKTTNPVATEAYGTVATNHQSAYAQAQTG | |
| | AVQSQGILPGMVWQDRDVYLQGPIWAKIPHTDGNFHPSPLMGGFGMKHP | |
| | PPQILIKNTPVPADPPTAFNKDKLNSFITQYSTGQVSVEIEWELQKENSKRW | |
| | NPEIQYTSNYYKSNNVEFAVNTEGVYSEPRPIGTRYLTRNL | |
| V105 (amino | MAADGYLPDWLEDNLSEGIREWWALKPGAPQPKANQQHQDNARGLVLP | 400 |
| acid) | GYKYLGPGNGLDKGEPVNAADAAALEHDKAYDQQLKAGDNPYLKYNHAD | |
| | AEFQERLKEDTSFGGNLGRAVFQAKKRLLEPLGLVEEAAKTAPGKKRPVE | |
| | QSPQEPDSSAGIGKSGAQPAKKRLNFGQTGDTESVPDPQPIGEPPAAPSG | |
| | VGSLTMASGGGAPVADNNEGADGVGSSSGNWHCDSQWLGDRVITTSTR | |
| | TWALPTYNNHLYKQISNSTSGGSSNDNAYFGYSTPWGYFDFNRFHCHFS | |
| | PRDWQRLINNNWGFRPKRLNFKLFNIQVKEVTDNNGVKTIANNLTSTVQVF | |
| | TDSDYQLPYVLGSAHEGCLPPFPADVFMIPQYGYLTLNDGSQAVGRSSFY | |
| | CLEYFPSQMLRTGNNFQFSYEFENVPFHSSYAHSQSLDRLMNPLIDQYLY | |
| | YLSKTINGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGSLIFG | |
| | KQGTGHDNVDADKVMITNEEEIKTTNPVATESYGTVATNHQSAYAQAQTG | |
| | AVQSQGILPGMVWQDRDVYLQGPIWAKIPHTDGNFHPSPLMGGFGMKHP | |
| | PPQILIKNTPVPADPPTAFNKDKLNSFITQYSTGQVSVEIEWELQKENSKRW | |
| | NPEIQYTSNYYKSNNVEFAVNTEGVYSEPRPIGTRYLTRNL | |
| V106 (amino | MAADGYLPDWLEDNLSEGIREWWALKPGAPQPKANQQHQDNARGLVLP | 401 |
| acid) | GYKYLGPGNGLDKGEPVNAADAAALEHDKAYDQQLKAGDNPYLKYNHAD | |
| | AEFQERLKEDTSFGGNLGRAVFQAKKRLLEPLGLVEEAAKTAPGKKRPVE | |
| | QSPQEPDSSAGIGKSGAQPAKKRLNFGQTGDTESVPDPQPIGEPPAAPSG | |
| | VGSLTMASGGGAPVADNNEGADGVGSSSGNWHCDSQWLGDRVITTSTR | |
| | TWALPTYNNHLYKQISNSTSGGSSNDNAYFGYSTPWGYFDFNRFHCHFS | |
| | PRDWQRLINNNWGFRPKRLNFKLFNIQVKEVTDNNGVKTIANNLTSTVQVF | |
| | TDSDYQLPYVLGSAHEGCLPPFPADVFMIPQYGYLTLNDGSQAVGRSSFY | |
| | CLEYFPSQMLRTGNNFQFSYEFENVPFHSSYAHSQSLDRLMNPLIDQYLY | |
| | YLSKTINGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGSLIFG | |
| | KQGTGHDNVDADKVMITNEEEIKTTNPVATESYGQVATNKQSAYAQAQTG | |
| | AVQSQGILPGMVWQDRDVYLQGPIWAKIPHTDGNFHPSPLMGGFGMKHP | |
| | PPQILIKNTPVPADPPTAFNKDKLNSFITQYSTGQVSVEIEWELQKENSKRW | |
| | NPEIQYTSNYYKSNNVEFAVNTEGVYSEPRPIGTRYLTRNL | |
| V107 (amino | MAADGYLPDWLEDNLSEGIREWWALKPGAPQPKANQQHQDNARGLVLP | 402 |
| acid) | GYKYLGPGNGLDKGEPVNAADAAALEHDKAYDQQLKAGDNPYLKYNHAD | |
| | AEFQERLKEDTSFGGNLGRAVFQAKKRLLEPLGLVEEAAKTAPGKKRPVE | |
| | QSPQEPDSSAGIGKSGAQPAKKRLNFGQTGDTESVPDPQPIGEPPAAPSG | |
| | VGSLTMASGGGAPVADNNEGADGVGSSSGNWHCDSQWLGDRVITTSTR | |
| | TWALPTYNNHLYKQISNSTSGGSSNDNAYFGYSTPWGYFDFNRFHCHFS | |
| | PRDWQRLINNNWGFRPKRLNFKLFNIQVKEVTDNNGVKTIANNLTSTVQVF | |
| | TDSDYQLPYVLGSAHEGCLPPFPADVFMIPQYGYLTLNDGSQAVGRSSFY | |
| | CLEYFPSQMLRTGNNFQFSYEFENVPFHSSYAHSQSLDRLMNPLIDQYLY | |
| | YLSKTINGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGSLIFG | |
| | KQGTGRDNVDADKVMITNEEEIKTTNPVATESYGQVATNHQSAYAQAQTG | |
| | ALQSQGVLPGMVWQDRDVYLQGPIWAKIPHTDGNFHPSPLMGGFGMKHP | |
| | PPQILIKNTPVPADPPTAFNKDKLNSFITQYSTGQVSVEIEWELQKENSKRW | |
| | NPEIQYTSNYYKSNNVEFAVNTEGVYSEPRPIGTRYLTRNL | |
| V108 (amino | MAADGYLPDWLEDNLSEGIREWWALKPGAPQPKANQQHQDNARGLVLP | 403 |
| acid) | GYKYLGPGNGLDKGEPVNAADAAALEHDKAYDQQLKAGDNPYLKYNHAD | |
| | AEFQERLKEDTSFGGNLGRAVFQAKKRLLEPLGLVEEAAKTAPGKKRPVE | |
| | QSPQEPDSSAGIGKSGAQPAKKRLNFGQTGDTESVPDPQPIGEPPAAPSG | |
| | VGSLTMASGGGAPVADNNEGADGVGSSSGNWHCDSQWLGDRVITTSTR | |
| | TWALPTYNNHLYKQISNSTSGGSSNDNAYFGYSTPWGYFDFNRFHCHFS | |
| | PRDWQRLINNNWGFRPKRLNFKLFNIQVKEVTDNNGVKTIANNLTSTVQVF | |
| | TDSDYQLPYVLGSAHEGCLPPFPADVFMIPQYGYLTLNDGSQAVGRSSFY | |
| | CLEYFPSQMLRTGNNFQFSYEFENVPFHSSYAHSQSLDRLMNPLIDQYLY | |
| | YLSKTINGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGSLIFG | |
| | KQGTGRDNVDADKVMITNEEEIKTTNPVATESYGQVATNKQSAYAQAQTG | |
| | AVQSQGVLPGMVWQDRDVYLQGPIWAKIPHTDGNFHPSPLMGGFGMKH | |
| | PPPQILIKNTPVPADPPTAFNKDKLNSFITQYSTGQVSVEIEWELQKENSKR | |
| | WNPEIQYTSNYYKSNNVEFAVNTEGVYSEPRPIGTRYLTRNL | |
| V109 (amino | MAADGYLPDWLEDNLSEGIREWWALKPGAPQPKANQQHQDNARGLVLP | 404 |
| acid) | GYKYLGPGNGLDKGEPVNAADAAALEHDKAYDQQLKAGDNPYLKYNHAD | |
| | AEFQERLKEDTSFGGNLGRAVFQAKKRLLEPLGLVEEAAKTAPGKKRPVE | |
| | QSPQEPDSSAGIGKSGAQPAKKRLNFGQTGDTESVPDPQPIGEPPAAPSG | |
| | VGSLTMASGGGAPVADNNEGADGVGSSSGNWHCDSQWLGDRVITTSTR | |
| | TWALPTYNNHLYKQISNSTSGGSSNDNAYFGYSTPWGYFDFNRFHCHFS | |
| | PRDWQRLINNNWGFRPKRLNFKLFNIQVKEVTDNNGVKTIANNLTSTVQVF | |
| | TDSDYQLPYVLGSAHEGCLPPFPADVFMIPQYGYLTLNDGSQAVGRSSFY | |
| | CLEYFPSQMLRTGNNFQFSYEFENVPFHSSYAHSQSLDRLMNPLIDQYLY | |
| | YLSKTINGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGSLIFG | |
| | KQGTGRDNVDADKVMITNEEEIKTTNPVATEAYGQVATNKQSAYAQAQTG | |
| | AVQNQGVLPGMVWQDRDVYLQGPIWAKIPHTDGNFHPSPLMGGFGMKH | |
| | PPPQILIKNTPVPADPPTAFNKDKLNSFITQYSTGQVSVEIEWELQKENSKR | |
| | WNPEIQYTSNYYKSNNVEFAVNTEGVYSEPRPIGTRYLTRNL | |
| V110 (amino | MAADGYLPDWLEDNLSEGIREWWALKPGAPQPKANQQHQDNARGLVLP | 405 |
| acid) | GYKYLGPGNGLDKGEPVNAADAAALEHDKAYDQQLKAGDNPYLKYNHAD | |
| | AEFQERLKEDTSFGGNLGRAVFQAKKRLLEPLGLVEEAAKTAPGKKRPVE | |
| | QSPQEPDSSAGIGKSGAQPAKKRLNFGQTGDTESVPDPQPIGEPPAAPSG | |
| | VGSLTMASGGGAPVADNNEGADGVGSSSGNWHCDSQWLGDRVITTSTR | |
| | TWALPTYNNHLYKQISNSTSGGSSNDNAYFGYSTPWGYFDFNRFHCHFS | |
| | PRDWQRLINNNWGFRPKRLNFKLFNIQVKEVTDNNGVKTIANNLTSTVQVF | |
| | TDSDYQLPYVLGSAHEGCLPPFPADVFMIPQYGYLTLNDGSQAVGRSSFY | |
| | CLEYFPSQMLRTGNNFQFSYEFENVPFHSSYAHSQSLDRLMNPLIDQYLY | |
| | YLSKTINGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGSLIFG | |
| | KQGTGRDNVDADKVMITNEEEIKTTNPVATESYGQVATNKQSAYAQAQTG | |
| | ALQSQGILPGMVWQDRDVYLQGPIWAKIPHTDGNFHPSPLMGGFGMKHP | |
| | PPQILIKNTPVPADPPTAFNKDKLNSFITQYSTGQVSVEIEWELQKENSKRW | |
| | NPEIQYTSNYYKSNNVEFAVNTEGVYSEPRPIGTRYLTRNL | |
| V111 (amino | MAADGYLPDWLEDNLSEGIREWWALKPGAPQPKANQQHQDNARGLVLP | 406 |
| acid) | GYKYLGPGNGLDKGEPVNAADAAALEHDKAYDQQLKAGDNPYLKYNHAD | |
| | AEFQERLKEDTSFGGNLGRAVFQAKKRLLEPLGLVEEAAKTAPGKKRPVE | |
| | QSPQEPDSSAGIGKSGAQPAKKRLNFGQTGDTESVPDPQPIGEPPAAPSG | |
| | VGSLTMASGGGAPVADNNEGADGVGSSSGNWHCDSQWLGDRVITTSTR | |
| | TWALPTYNNHLYKQISNSTSGGSSNDNAYFGYSTPWGYFDFNRFHCHFS | |
| | PRDWQRLINNNWGFRPKRLNFKLFNIQVKEVTDNNGVKTIANNLTSTVQVF | |
| | TDSDYQLPYVLGSAHEGCLPPFPADVFMIPQYGYLTLNDGSQAVGRSSFY | |
| | CLEYFPSQMLRTGNNFQFSYEFENVPFHSSYAHSQSLDRLMNPLIDQYLY | |
| | YLSKTINGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGSLIFG | |
| | KQGTGHDNVDADKVMITNEEEIKTTNPVATESYGTVATNHQSAYAQAQTG | |
| | AVQNQGVLPGMVWQDRDVYLQGPIWAKIPHTDGNFHPSPLMGGFGMKH | |
| | PPPQILIKNTPVPADPPTAFNKDKLNSFITQYSTGQVSVEIEWELQKENSKR | |
| | WNPEIQYTSNYYKSNNVEFAVNTEGVYSEPRPIGTRYLTRNL | |
| V112 (amino | MAADGYLPDWLEDNLSEGIREWWALKPGAPQPKANQQHQDNARGLVLP | 407 |
| acid) | GYKYLGPGNGLDKGEPVNAADAAALEHDKAYDQQLKAGDNPYLKYNHAD | |
| | AEFQERLKEDTSFGGNLGRAVFQAKKRLLEPLGLVEEAAKTAPGKKRPVE | |
| | QSPQEPDSSAGIGKSGAQPAKKRLNFGQTGDTESVPDPQPIGEPPAAPSG | |
| | VGSLTMASGGGAPVADNNEGADGVGSSSGNWHCDSQWLGDRVITTSTR | |
| | TWALPTYNNHLYKQISNSTSGGSSNDNAYFGYSTPWGYFDFNRFHCHFS | |
| | PRDWQRLINNNWGFRPKRLNFKLFNIQVKEVTDNNGVKTIANNLTSTVQVF | |
| | TDSDYQLPYVLGSAHEGCLPPFPADVFMIPQYGYLTLNDGSQAVGRSSFY | |
| | CLEYFPSQMLRTGNNFQFSYEFENVPFHSSYAHSQSLDRLMNPLIDQYLY | |
| | YLSKTINGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGSLIFG | |
| | KQGTGRDNVDADKVMITNEEEIKTTNPVATESYGQVATNKQSAYAQAQTG | |
| | ALQNQGILPGMVWQDRDVYLQGPIWAKIPHTDGNFHPSPLMGGFGMKHP | |
| | PPQILIKNTPVPADPPTAFNKDKLNSFITQYSTGQVSVEIEWELQKENSKRW | |
| | NPEIQYTSNYYKSNNVEFAVNTEGVYSEPRPIGTRYLTRNL | |
| V113 (amino | MAADGYLPDWLEDNLSEGIREWWALKPGAPQPKANQQHQDNARGLVLP | 408 |
| acid) | GYKYLGPGNGLDKGEPVNAADAAALEHDKAYDQQLKAGDNPYLKYNHAD | |
| | AEFQERLKEDTSFGGNLGRAVFQAKKRLLEPLGLVEEAAKTAPGKKRPVE | |
| | QSPQEPDSSAGIGKSGAQPAKKRLNFGQTGDTESVPDPQPIGEPPAAPSG | |
| | VGSLTMASGGGAPVADNNEGADGVGSSSGNWHCDSQWLGDRVITTSTR | |
| | TWALPTYNNHLYKQISNSTSGGSSNDNAYFGYSTPWGYFDFNRFHCHFS | |
| | PRDWQRLINNNWGFRPKRLNFKLFNIQVKEVTDNNGVKTIANNLTSTVQVF | |
| | TDSDYQLPYVLGSAHEGCLPPFPADVFMIPQYGYLTLNDGSQAVGRSSFY | |
| | CLEYFPSQMLRTGNNFQFSYEFENVPFHSSYAHSQSLDRLMNPLIDQYLY | |
| | YLSKTINGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGSLIFG | |
| | KQGTGRDNVDADKVMITNEEEIKTTNPVATESYGQVATNKQSAYAQAQTG | |
| | AVQSQGILPGMVWQDRDVYLQGPIWAKIPHTDGNFHPSPLMGGFGMKHP | |
| | PPQILIKNTPVPADPPTAFNKDKLNSFITQYSTGQVSVEIEWELQKENSKRW | |
| | NPEIQYTSNYYKSNNVEFAVNTEGVYSEPRPIGTRYLTRNL | |
| V114 (amino | MAADGYLPDWLEDNLSEGIREWWALKPGAPQPKANQQHQDNARGLVLP | 409 |
| acid) | GYKYLGPGNGLDKGEPVNAADAAALEHDKAYDQQLKAGDNPYLKYNHAD | |
| | AEFQERLKEDTSFGGNLGRAVFQAKKRLLEPLGLVEEAAKTAPGKKRPVE | |
| | QSPQEPDSSAGIGKSGAQPAKKRLNFGQTGDTESVPDPQPIGEPPAAPSG | |
| | VGSLTMASGGGAPVADNNEGADGVGSSSGNWHCDSQWLGDRVITTSTR | |
| | TWALPTYNNHLYKQISNSTSGGSSNDNAYFGYSTPWGYFDFNRFHCHFS | |
| | PRDWQRLINNNWGFRPKRLNFKLFNIQVKEVTDNNGVKTIANNLTSTVQVF | |
| | TDSDYQLPYVLGSAHEGCLPPFPADVFMIPQYGYLTLNDGSQAVGRSSFY | |
| | CLEYFPSQMLRTGNNFQFSYEFENVPFHSSYAHSQSLDRLMNPLIDQYLY | |
| | YLSKTINGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGSLIFG | |
| | KQGTGRDNVDADKVMITNEEEIKTTNPVATESYGQVATNHQSAYAQAQTG | |
| | ALQNQGVLPGMVWQDRDVYLQGPIWAKIPHTDGNFHPSPLMGGFGMKH | |
| | PPPQILIKNTPVPADPPTAFNKDKLNSFITQYSTGQVSVEIEWELQKENSKR | |
| | WNPEIQYTSNYYKSNNVEFAVNTEGVYSEPRPIGTRYLTRNL | |
| V115 (amino | MAADGYLPDWLEDNLSEGIREWWALKPGAPQPKANQQHQDNARGLVLP | 410 |
| acid) | GYKYLGPGNGLDKGEPVNAADAAALEHDKAYDQQLKAGDNPYLKYNHAD | |
| | AEFQERLKEDTSFGGNLGRAVFQAKKRLLEPLGLVEEAAKTAPGKKRPVE | |
| | QSPQEPDSSAGIGKSGAQPAKKRLNFGQTGDTESVPDPQPIGEPPAAPSG | |
| | VGSLTMASGGGAPVADNNEGADGVGSSSGNWHCDSQWLGDRVITTSTR | |
| | TWALPTYNNHLYKQISNSTSGGSSNDNAYFGYSTPWGYFDFNRFHCHFS | |
| | PRDWQRLINNNWGFRPKRLNFKLFNIQVKEVTDNNGVKTIANNLTSTVQVF | |
| | TDSDYQLPYVLGSAHEGCLPPFPADVFMIPQYGYLTLNDGSQAVGRSSFY | |
| | CLEYFPSQMLRTGNNFQFSYEFENVPFHSSYAHSQSLDRLMNPLIDQYLY | |
| | YLSKTINGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGSLIFG | |
| | KQGTGHDNVDADKVMITNEEEIKTTNPVATESYGQVATNHQSAYAQAQTG | |
| | AVQSQGILPGMVWQDRDVYLQGPIWAKIPHTDGNFHPSPLMGGFGMKHP | |
| | PPQILIKNTPVPADPPTAFNKDKLNSFITQYSTGQVSVEIEWELQKENSKRW | |
| | NPEIQYTSNYYKSNNVEFAVNTEGVYSEPRPIGTRYLTRNL | |
| V116 (amino | MAADGYLPDWLEDNLSEGIREWWALKPGAPQPKANQQHQDNARGLVLP | 411 |
| acid) | GYKYLGPGNGLDKGEPVNAADAAALEHDKAYDQQLKAGDNPYLKYNHAD | |
| | AEFQERLKEDTSFGGNLGRAVFQAKKRLLEPLGLVEEAAKTAPGKKRPVE | |
| | QSPQEPDSSAGIGKSGAQPAKKRLNFGQTGDTESVPDPQPIGEPPAAPSG | |
| | VGSLTMASGGGAPVADNNEGADGVGSSSGNWHCDSQWLGDRVITTSTR | |
| | TWALPTYNNHLYKQISNSTSGGSSNDNAYFGYSTPWGYFDFNRFHCHFS | |
| | PRDWQRLINNNWGFRPKRLNFKLFNIQVKEVTDNNGVKTIANNLTSTVQVF | |
| | TDSDYQLPYVLGSAHEGCLPPFPADVFMIPQYGYLTLNDGSQAVGRSSFY | |
| | CLEYFPSQMLRTGNNFQFSYEFENVPFHSSYAHSQSLDRLMNPLIDQYLY | |
| | YLSKTINGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGSLIFG | |
| | KQGTGRDNVDADKVMITNEEEIKTTNPVATEAYGQVATNHQSAYAQAQTG | |
| | ALQNQGILPGMVWQDRDVYLQGPIWAKIPHTDGNFHPSPLMGGFGMKHP | |
| | PPQILIKNTPVPADPPTAFNKDKLNSFITQYSTGQVSVEIEWELQKENSKRW | |
| | NPEIQYTSNYYKSNNVEFAVNTEGVYSEPRPIGTRYLTRNL | |
| V117 (amino | MAADGYLPDWLEDNLSEGIREWWALKPGAPQPKANQQHQDNARGLVLP | 412 |
| acid) | GYKYLGPGNGLDKGEPVNAADAAALEHDKAYDQQLKAGDNPYLKYNHAD | |
| | AEFQERLKEDTSFGGNLGRAVFQAKKRLLEPLGLVEEAAKTAPGKKRPVE | |
| | QSPQEPDSSAGIGKSGAQPAKKRLNFGQTGDTESVPDPQPIGEPPAAPSG | |
| | VGSLTMASGGGAPVADNNEGADGVGSSSGNWHCDSQWLGDRVITTSTR | |
| | TWALPTYNNHLYKQISNSTSGGSSNDNAYFGYSTPWGYFDFNRFHCHFS | |
| | PRDWQRLINNNWGFRPKRLNFKLFNIQVKEVTDNNGVKTIANNLTSTVQVF | |
| | TDSDYQLPYVLGSAHEGCLPPFPADVFMIPQYGYLTLNDGSQAVGRSSFY | |
| | CLEYFPSQMLRTGNNFQFSYEFENVPFHSSYAHSQSLDRLMNPLIDQYLY | |
| | YLSKTINGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGSLIFG | |
| | KQGTGRDNVDADKVMITNEEEIKTTNPVATESYGTVATNHQSAYAQAQTG | |
| | ALQNQGILPGMVWQDRDVYLQGPIWAKIPHTDGNFHPSPLMGGFGMKHP | |
| | PPQILIKNTPVPADPPTAFNKDKLNSFITQYSTGQVSVEIEWELQKENSKRW | |
| | NPEIQYTSNYYKSNNVEFAVNTEGVYSEPRPIGTRYLTRNL | |
| V118 (amino | MAADGYLPDWLEDNLSEGIREWWALKPGAPQPKANQQHQDNARGLVLP | 413 |
| acid) | GYKYLGPGNGLDKGEPVNAADAAALEHDKAYDQQLKAGDNPYLKYNHAD | |
| | AEFQERLKEDTSFGGNLGRAVFQAKKRLLEPLGLVEEAAKTAPGKKRPVE | |
| | QSPQEPDSSAGIGKSGAQPAKKRLNFGQTGDTESVPDPQPIGEPPAAPSG | |
| | VGSLTMASGGGAPVADNNEGADGVGSSSGNWHCDSQWLGDRVITTSTR | |
| | TWALPTYNNHLYKQISNSTSGGSSNDNAYFGYSTPWGYFDFNRFHCHFS | |
| | PRDWQRLINNNWGFRPKRLNFKLFNIQVKEVTDNNGVKTIANNLTSTVQVF | |
| | TDSDYQLPYVLGSAHEGCLPPFPADVFMIPQYGYLTLNDGSQAVGRSSFY | |
| | CLEYFPSQMLRTGNNFQFSYEFENVPFHSSYAHSQSLDRLMNPLIDQYLY | |
| | YLSKTINGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGSLIFG | |
| | KQGTGRDNVDADKVMITNEEEIKTTNPVATESYGTVATNHQSAQAQAQTG | |
| | WLQSQGVLPGMVWQDRDVYLQGPIWAKIPHTDGNFHPSPLMGGFGMKH | |
| | PPPQILIKNTPVPADPPTAFNKDKLNSFITQYSTGQVSVEIEWELQKENSKR | |
| | WNPEIQYTSNYYKSNNVEFAVNTEGVYSEPRPIGTRYLTRNL | |
| V119 (amino | MAADGYLPDWLEDNLSEGIREWWALKPGAPQPKANQQHQDNARGLVLP | 414 |
| acid) | GYKYLGPGNGLDKGEPVNAADAAALEHDKAYDQQLKAGDNPYLKYNHAD | |
| | AEFQERLKEDTSFGGNLGRAVFQAKKRLLEPLGLVEEAAKTAPGKKRPVE | |
| | QSPQEPDSSAGIGKSGAQPAKKRLNFGQTGDTESVPDPQPIGEPPAAPSG | |
| | VGSLTMASGGGAPVADNNEGADGVGSSSGNWHCDSQWLGDRVITTSTR | |
| | TWALPTYNNHLYKQISNSTSGGSSNDNAYFGYSTPWGYFDFNRFHCHFS | |
| | PRDWQRLINNNWGFRPKRLNFKLFNIQVKEVTDNNGVKTIANNLTSTVQVF | |
| | TDSDYQLPYVLGSAHEGCLPPFPADVFMIPQYGYLTLNDGSQAVGRSSFY | |
| | CLEYFPSQMLRTGNNFQFSYEFENVPFHSSYAHSQSLDRLMNPLIDQYLY | |
| | YLSKTINGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGSLIFG | |
| | KQGTGHDNVDADKVMITNEEEIKTTNPVATESYGTVATNHQSAYAQAQTG | |
| | AVQNQGILPGMVWQDRDVYLQGPIWAKIPHTDGNFHPSPLMGGFGMKHP | |
| | PPQILIKNTPVPADPPTAFNKDKLNSFITQYSTGQVSVEIEWELQKENSKRW | |
| | NPEIQYTSNYYKSNNVEFAVNTEGVYSEPRPIGTRYLTRNL | |
| V120 (amino | MAADGYLPDWLEDNLSEGIREWWALKPGAPQPKANQQHQDNARGLVLP | 415 |
| acid) | GYKYLGPGNGLDKGEPVNAADAAALEHDKAYDQQLKAGDNPYLKYNHAD | |
| | AEFQERLKEDTSFGGNLGRAVFQAKKRLLEPLGLVEEAAKTAPGKKRPVE | |
| | QSPQEPDSSAGIGKSGAQPAKKRLNFGQTGDTESVPDPQPIGEPPAAPSG | |
| | VGSLTMASGGGAPVADNNEGADGVGSSSGNWHCDSQWLGDRVITTSTR | |
| | TWALPTYNNHLYKQISNSTSGGSSNDNAYFGYSTPWGYFDFNRFHCHFS | |
| | PRDWQRLINNNWGFRPKRLNFKLFNIQVKEVTDNNGVKTIANNLTSTVQVF | |
| | TDSDYQLPYVLGSAHEGCLPPFPADVFMIPQYGYLTLNDGSQAVGRSSFY | |
| | CLEYFPSQMLRTGNNFQFSYEFENVPFHSSYAHSQSLDRLMNPLIDQYLY | |
| | YLSKTINGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGSLIFG | |
| | KQGTGRDNVDADKVMITNEEEIKTTNPVATESYGQVATNHQSAYAQAQTG | |
| | ALQSQGILPGMVWQDRDVYLQGPIWAKIPHTDGNFHPSPLMGGFGMKHP | |
| | PPQILIKNTPVPADPPTAFNKDKLNSFITQYSTGQVSVEIEWELQKENSKRW | |
| | NPEIQYTSNYYKSNNVEFAVNTEGVYSEPRPIGTRYLTRNL | |
| V121 (amino | MAADGYLPDWLEDNLSEGIREWWALKPGAPQPKANQQHQDNARGLVLP | 416 |
| acid) | GYKYLGPGNGLDKGEPVNAADAAALEHDKAYDQQLKAGDNPYLKYNHAD | |
| | AEFQERLKEDTSFGGNLGRAVFQAKKRLLEPLGLVEEAAKTAPGKKRPVE | |
| | QSPQEPDSSAGIGKSGAQPAKKRLNFGQTGDTESVPDPQPIGEPPAAPSG | |
| | VGSLTMASGGGAPVADNNEGADGVGSSSGNWHCDSQWLGDRVITTSTR | |
| | TWALPTYNNHLYKQISNSTSGGSSNDNAYFGYSTPWGYFDFNRFHCHFS | |
| | PRDWQRLINNNWGFRPKRLNFKLFNIQVKEVTDNNGVKTIANNLTSTVQVF | |
| | TDSDYQLPYVLGSAHEGCLPPFPADVFMIPQYGYLTLNDGSQAVGRSSFY | |
| | CLEYFPSQMLRTGNNFQFSYEFENVPFHSSYAHSQSLDRLMNPLIDQYLY | |
| | YLSKTINGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGSLIFG | |
| | KQGTGRDNVDADKVMITNEEEIKTTNPVATESYGTVATNKQSAYAQAQTG | |
| | AVQNQGILPGMVWQDRDVYLQGPIWAKIPHTDGNFHPSPLMGGFGMKHP | |
| | PPQILIKNTPVPADPPTAFNKDKLNSFITQYSTGQVSVEIEWELQKENSKRW | |
| | NPEIQYTSNYYKSNNVEFAVNTEGVYSEPRPIGTRYLTRNL | |
| V122 (amino | MAADGYLPDWLEDNLSEGIREWWALKPGAPQPKANQQHQDNARGLVLP | 417 |
| acid) | GYKYLGPGNGLDKGEPVNAADAAALEHDKAYDQQLKAGDNPYLKYNHAD | |
| | AEFQERLKEDTSFGGNLGRAVFQAKKRLLEPLGLVEEAAKTAPGKKRPVE | |
| | QSPQEPDSSAGIGKSGAQPAKKRLNFGQTGDTESVPDPQPIGEPPAAPSG | |
| | VGSLTMASGGGAPVADNNEGADGVGSSSGNWHCDSQWLGDRVITTSTR | |
| | TWALPTYNNHLYKQISNSTSGGSSNDNAYFGYSTPWGYFDFNRFHCHFS | |
| | PRDWQRLINNNWGFRPKRLNFKLFNIQVKEVTDNNGVKTIANNLTSTVQVF | |
| | TDSDYQLPYVLGSAHEGCLPPFPADVFMIPQYGYLTLNDGSQAVGRSSFY | |
| | CLEYFPSQMLRTGNNFQFSYEFENVPFHSSYAHSQSLDRLMNPLIDQYLY | |
| | YLSKTINGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGSLIFG | |
| | KQGTGRDNVDADKVMITNEEEIKTTNPVATESYGTVATNHQSAYAQAQTG | |
| | AVQNQGVLPGMVWQDRDVYLQGPIWAKIPHTDGNFHPSPLMGGFGMKH | |
| | PPPQILIKNTPVPADPPTAFNKDKLNSFITQYSTGQVSVEIEWELQKENSKR | |
| | WNPEIQYTSNYYKSNNVEFAVNTEGVYSEPRPIGTRYLTRNL | |
| V123 (amino | MAADGYLPDWLEDNLSEGIREWWALKPGAPQPKANQQHQDNARGLVLP | 418 |
| acid) | GYKYLGPGNGLDKGEPVNAADAAALEHDKAYDQQLKAGDNPYLKYNHAD | |
| | AEFQERLKEDTSFGGNLGRAVFQAKKRLLEPLGLVEEAAKTAPGKKRPVE | |
| | QSPQEPDSSAGIGKSGAQPAKKRLNFGQTGDTESVPDPQPIGEPPAAPSG | |
| | VGSLTMASGGGAPVADNNEGADGVGSSSGNWHCDSQWLGDRVITTSTR | |
| | TWALPTYNNHLYKQISNSTSGGSSNDNAYFGYSTPWGYFDFNRFHCHFS | |
| | PRDWQRLINNNWGFRPKRLNFKLFNIQVKEVTDNNGVKTIANNLTSTVQVF | |
| | TDSDYQLPYVLGSAHEGCLPPFPADVFMIPQYGYLTLNDGSQAVGRSSFY | |
| | CLEYFPSQMLRTGNNFQFSYEFENVPFHSSYAHSQSLDRLMNPLIDQYLY | |
| | YLSKTINGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGSLIFG | |
| | KQGTGRDNVDADKVMITNEEEIKTTNPVATESYGTVATNHQSAYAQAQTG | |
| | AVQSQGILPGMVWQDRDVYLQGPIWAKIPHTDGNFHPSPLMGGFGMKHP | |
| | PPQILIKNTPVPADPPTAFNKDKLNSFITQYSTGQVSVEIEWELQKENSKRW | |
| | NPEIQYTSNYYKSNNVEFAVNTEGVYSEPRPIGTRYLTRNL | |
| V124 (amino | MAADGYLPDWLEDNLSEGIREWWALKPGAPQPKANQQHQDNARGLVLP | 419 |
| acid) | GYKYLGPGNGLDKGEPVNAADAAALEHDKAYDQQLKAGDNPYLKYNHAD | |
| | AEFQERLKEDTSFGGNLGRAVFQAKKRLLEPLGLVEEAAKTAPGKKRPVE | |
| | QSPQEPDSSAGIGKSGAQPAKKRLNFGQTGDTESVPDPQPIGEPPAAPSG | |
| | VGSLTMASGGGAPVADNNEGADGVGSSSGNWHCDSQWLGDRVITTSTR | |
| | TWALPTYNNHLYKQISNSTSGGSSNDNAYFGYSTPWGYFDFNRFHCHFS | |
| | PRDWQRLINNNWGFRPKRLNFKLFNIQVKEVTDNNGVKTIANNLTSTVQVF | |
| | TDSDYQLPYVLGSAHEGCLPPFPADVFMIPQYGYLTLNDGSQAVGRSSFY | |
| | CLEYFPSQMLRTGNNFQFSYEFENVPFHSSYAHSQSLDRLMNPLIDQYLY | |
| | YLSKTINGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGSLIFG | |
| | KQGTGRDNVDADKVMITNEEEIKTTNPVATESYGQVATNKQSAYAQAQTG | |
| | AVQNQGVLPGMVWQDRDVYLQGPIWAKIPHTDGNFHPSPLMGGFGMKH | |
| | PPPQILIKNTPVPADPPTAFNKDKLNSFITQYSTGQVSVEIEWELQKENSKR | |
| | WNPEIQYTSNYYKSNNVEFAVNTEGVYSEPRPIGTRYLTRNL | |
| V125 (amino | MAADGYLPDWLEDNLSEGIREWWALKPGAPQPKANQQHQDNARGLVLP | 420 |
| acid) | GYKYLGPGNGLDKGEPVNAADAAALEHDKAYDQQLKAGDNPYLKYNHAD | |
| | AEFQERLKEDTSFGGNLGRAVFQAKKRLLEPLGLVEEAAKTAPGKKRPVE | |
| | QSPQEPDSSAGIGKSGAQPAKKRLNFGQTGDTESVPDPQPIGEPPAAPSG | |
| | VGSLTMASGGGAPVADNNEGADGVGSSSGNWHCDSQWLGDRVITTSTR | |
| | TWALPTYNNHLYKQISNSTSGGSSNDNAYFGYSTPWGYFDFNRFHCHFS | |
| | PRDWQRLINNNWGFRPKRLNFKLFNIQVKEVTDNNGVKTIANNLTSTVQVF | |
| | TDSDYQLPYVLGSAHEGCLPPFPADVFMIPQYGYLTLNDGSQAVGRSSFY | |
| | CLEYFPSQMLRTGNNFQFSYEFENVPFHSSYAHSQSLDRLMNPLIDQYLY | |
| | YLSKTINGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGSLIFG | |
| | KQGTGHDNVDADKVMITNEEEIKTTNPVATEAYGQVATNHQSAYAQAQTG | |
| | AVQNQGILPGMVWQDRDVYLQGPIWAKIPHTDGNFHPSPLMGGFGMKHP | |
| | PPQILIKNTPVPADPPTAFNKDKLNSFITQYSTGQVSVEIEWELQKENSKRW | |
| | NPEIQYTSNYYKSNNVEFAVNTEGVYSEPRPIGTRYLTRNL | |
| V126 (amino | MAADGYLPDWLEDNLSEGIREWWALKPGAPQPKANQQHQDNARGLVLP | 421 |
| acid) | GYKYLGPGNGLDKGEPVNAADAAALEHDKAYDQQLKAGDNPYLKYNHAD | |
| | AEFQERLKEDTSFGGNLGRAVFQAKKRLLEPLGLVEEAAKTAPGKKRPVE | |
| | QSPQEPDSSAGIGKSGAQPAKKRLNFGQTGDTESVPDPQPIGEPPAAPSG | |
| | VGSLTMASGGGAPVADNNEGADGVGSSSGNWHCDSQWLGDRVITTSTR | |
| | TWALPTYNNHLYKQISNSTSGGSSNDNAYFGYSTPWGYFDFNRFHCHFS | |
| | PRDWQRLINNNWGFRPKRLNFKLFNIQVKEVTDNNGVKTIANNLTSTVQVF | |
| | TDSDYQLPYVLGSAHEGCLPPFPADVFMIPQYGYLTLNDGSQAVGRSSFY | |
| | CLEYFPSQMLRTGNNFQFSYEFENVPFHSSYAHSQSLDRLMNPLIDQYLY | |
| | YLSKTINGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGSLIFG | |
| | KQGTGRDNVDADKVMITNEEEIKTTNPVATEAYGTVATNHQSAYAQAQTG | |
| | AVQNQGILPGMVWQDRDVYLQGPIWAKIPHTDGNFHPSPLMGGFGMKHP | |
| | PPQILIKNTPVPADPPTAFNKDKLNSFITQYSTGQVSVEIEWELQKENSKRW | |
| | NPEIQYTSNYYKSNNVEFAVNTEGVYSEPRPIGTRYLTRNL | |
| V127 (amino | MAADGYLPDWLEDNLSEGIREWWALKPGAPQPKANQQHQDNARGLVLP | 422 |
| acid) | GYKYLGPGNGLDKGEPVNAADAAALEHDKAYDQQLKAGDNPYLKYNHAD | |
| | AEFQERLKEDTSFGGNLGRAVFQAKKRLLEPLGLVEEAAKTAPGKKRPVE | |
| | QSPQEPDSSAGIGKSGAQPAKKRLNFGQTGDTESVPDPQPIGEPPAAPSG | |
| | VGSLTMASGGGAPVADNNEGADGVGSSSGNWHCDSQWLGDRVITTSTR | |
| | TWALPTYNNHLYKQISNSTSGGSSNDNAYFGYSTPWGYFDFNRFHCHFS | |
| | PRDWQRLINNNWGFRPKRLNFKLFNIQVKEVTDNNGVKTIANNLTSTVQVF | |
| | TDSDYQLPYVLGSAHEGCLPPFPADVFMIPQYGYLTLNDGSQAVGRSSFY | |
| | CLEYFPSQMLRTGNNFQFSYEFENVPFHSSYAHSQSLDRLMNPLIDQYLY | |
| | YLSKTINGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGSLIFG | |
| | KQGTGRDNVDADKVMITNEEEIKTTNPVATEAYGQVATNHQSAYAQAQTG | |
| | AVQSQGILPGMVWQDRDVYLQGPIWAKIPHTDGNFHPSPLMGGFGMKHP | |
| | PPQILIKNTPVPADPPTAFNKDKLNSFITQYSTGQVSVEIEWELQKENSKRW | |
| | NPEIQYTSNYYKSNNVEFAVNTEGVYSEPRPIGTRYLTRNL | |
| V128 (amino | MAADGYLPDWLEDNLSEGIREWWALKPGAPQPKANQQHQDNARGLVLP | 423 |
| acid) | GYKYLGPGNGLDKGEPVNAADAAALEHDKAYDQQLKAGDNPYLKYNHAD | |
| | AEFQERLKEDTSFGGNLGRAVFQAKKRLLEPLGLVEEAAKTAPGKKRPVE | |
| | QSPQEPDSSAGIGKSGAQPAKKRLNFGQTGDTESVPDPQPIGEPPAAPSG | |
| | VGSLTMASGGGAPVADNNEGADGVGSSSGNWHCDSQWLGDRVITTSTR | |
| | TWALPTYNNHLYKQISNSTSGGSSNDNAYFGYSTPWGYFDFNRFHCHFS | |
| | PRDWQRLINNNWGFRPKRLNFKLFNIQVKEVTDNNGVKTIANNLTSTVQVF | |
| | TDSDYQLPYVLGSAHEGCLPPFPADVFMIPQYGYLTLNDGSQAVGRSSFY | |
| | CLEYFPSQMLRTGNNFQFSYEFENVPFHSSYAHSQSLDRLMNPLIDQYLY | |
| | YLSKTINGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGSLIFG | |
| | KQGTGRDNVDADKVMITNEEEIKTTNPVATEAYGQVATNHQSAYAQAQTG | |
| | AVQNQGVLPGMVWQDRDVYLQGPIWAKIPHTDGNFHPSPLMGGFGMKH | |
| | PPPQILIKNTPVPADPPTAFNKDKLNSFITQYSTGQVSVEIEWELQKENSKR | |
| | WNPEIQYTSNYYKSNNVEFAVNTEGVYSEPRPIGTRYLTRNL | |
| V129 (amino | MAADGYLPDWLEDNLSEGIREWWALKPGAPQPKANQQHQDNARGLVLP | 424 |
| acid) | GYKYLGPGNGLDKGEPVNAADAAALEHDKAYDQQLKAGDNPYLKYNHAD | |
| | AEFQERLKEDTSFGGNLGRAVFQAKKRLLEPLGLVEEAAKTAPGKKRPVE | |
| | QSPQEPDSSAGIGKSGAQPAKKRLNFGQTGDTESVPDPQPIGEPPAAPSG | |
| | VGSLTMASGGGAPVADNNEGADGVGSSSGNWHCDSQWLGDRVITTSTR | |
| | TWALPTYNNHLYKQISNSTSGGSSNDNAYFGYSTPWGYFDFNRFHCHFS | |
| | PRDWQRLINNNWGFRPKRLNFKLFNIQVKEVTDNNGVKTIANNLTSTVQVF | |
| | TDSDYQLPYVLGSAHEGCLPPFPADVFMIPQYGYLTLNDGSQAVGRSSFY | |
| | CLEYFPSQMLRTGNNFQFSYEFENVPFHSSYAHSQSLDRLMNPLIDQYLY | |
| | YLSKTINGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGSLIFG | |
| | KQGTGRDNVDADKVMITNEEEIKTTNPVATEAYGQVATNKQSAYAQAQTG | |
| | AVQNQGILPGMVWQDRDVYLQGPIWAKIPHTDGNFHPSPLMGGFGMKHP | |
| | PPQILIKNTPVPADPPTAFNKDKLNSFITQYSTGQVSVEIEWELQKENSKRW | |
| | NPEIQYTSNYYKSNNVEFAVNTEGVYSEPRPIGTRYLTRNL | |
| V130 (amino | MAADGYLPDWLEDNLSEGIREWWALKPGAPQPKANQQHQDNARGLVLP | 425 |
| acid) | GYKYLGPGNGLDKGEPVNAADAAALEHDKAYDQQLKAGDNPYLKYNHAD | |
| | AEFQERLKEDTSFGGNLGRAVFQAKKRLLEPLGLVEEAAKTAPGKKRPVE | |
| | QSPQEPDSSAGIGKSGAQPAKKRLNFGQTGDTESVPDPQPIGEPPAAPSG | |
| | VGSLTMASGGGAPVADNNEGADGVGSSSGNWHCDSQWLGDRVITTSTR | |
| | TWALPTYNNHLYKQISNSTSGGSSNDNAYFGYSTPWGYFDFNRFHCHFS | |
| | PRDWQRLINNNWGFRPKRLNFKLFNIQVKEVTDNNGVKTIANNLTSTVQVF | |
| | TDSDYQLPYVLGSAHEGCLPPFPADVFMIPQYGYLTLNDGSQAVGRSSFY | |
| | CLEYFPSQMLRTGNNFQFSYEFENVPFHSSYAHSQSLDRLMNPLIDQYLY | |
| | YLSKTINGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGSLIFG | |
| | KQGTGHDNVDADKVMITNEEEIKTTNPVATESYGQVATNHQSAYAQAQTG | |
| | AVQNQGVLPGMVWQDRDVYLQGPIWAKIPHTDGNFHPSPLMGGFGMKH | |
| | PPPQILIKNTPVPADPPTAFNKDKLNSFITQYSTGQVSVEIEWELQKENSKR | |
| | WNPEIQYTSNYYKSNNVEFAVNTEGVYSEPRPIGTRYLTRNL | |
| V131 (amino | MAADGYLPDWLEDNLSEGIREWWALKPGAPQPKANQQHQDNARGLVLP | 426 |
| acid) | GYKYLGPGNGLDKGEPVNAADAAALEHDKAYDQQLKAGDNPYLKYNHAD | |
| | AEFQERLKEDTSFGGNLGRAVFQAKKRLLEPLGLVEEAAKTAPGKKRPVE | |
| | QSPQEPDSSAGIGKSGAQPAKKRLNFGQTGDTESVPDPQPIGEPPAAPSG | |
| | VGSLTMASGGGAPVADNNEGADGVGSSSGNWHCDSQWLGDRVITTSTR | |
| | TWALPTYNNHLYKQISNSTSGGSSNDNAYFGYSTPWGYFDFNRFHCHFS | |
| | PRDWQRLINNNWGFRPKRLNFKLFNIQVKEVTDNNGVKTIANNLTSTVQVF | |
| | TDSDYQLPYVLGSAHEGCLPPFPADVFMIPQYGYLTLNDGSQAVGRSSFY | |
| | CLEYFPSQMLRTGNNFQFSYEFENVPFHSSYAHSQSLDRLMNPLIDQYLY | |
| | YLSKTINGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGSLIFG | |
| | KQGTGRDNVDADKVMITNEEEIKTTNPVATESYGTVATNHQSAQAQAQTG | |
| | ALQNQGILPGMVWQDRDVYLQGPIWAKIPHTDGNFHPSPLMGGFGMKHP | |
| | PPQILIKNTPVPADPPTAFNKDKLNSFITQYSTGQVSVEIEWELQKENSKRW | |
| | NPEIQYTSNYYKSNNVEFAVNTEGVYSEPRPIGTRYLTRNL | |
| V132 (amino | MAADGYLPDWLEDNLSEGIREWWALKPGAPQPKANQQHQDNARGLVLP | 427 |
| acid) | GYKYLGPGNGLDKGEPVNAADAAALEHDKAYDQQLKAGDNPYLKYNHAD | |
| | AEFQERLKEDTSFGGNLGRAVFQAKKRLLEPLGLVEEAAKTAPGKKRPVE | |
| | QSPQEPDSSAGIGKSGAQPAKKRLNFGQTGDTESVPDPQPIGEPPAAPSG | |
| | VGSLTMASGGGAPVADNNEGADGVGSSSGNWHCDSQWLGDRVITTSTR | |
| | TWALPTYNNHLYKQISNSTSGGSSNDNAYFGYSTPWGYFDFNRFHCHFS | |
| | PRDWQRLINNNWGFRPKRLNFKLFNIQVKEVTDNNGVKTIANNLTSTVQVF | |
| | TDSDYQLPYVLGSAHEGCLPPFPADVFMIPQYGYLTLNDGSQAVGRSSFY | |
| | CLEYFPSQMLRTGNNFQFSYEFENVPFHSSYAHSQSLDRLMNPLIDQYLY | |
| | YLSKTINGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGSLIFG | |
| | KQGTGRDNVDADKVMITNEEEIKTTNPVATESYGQVATNHQSAYAQAQTG | |
| | WLQSQGILPGMVWQDRDVYLQGPIWAKIPHTDGNFHPSPLMGGFGMKHP | |
| | PPQILIKNTPVPADPPTAFNKDKLNSFITQYSTGQVSVEIEWELQKENSKRW | |
| | NPEIQYTSNYYKSNNVEFAVNTEGVYSEPRPIGTRYLTRNL | |
| V133 (amino | MAADGYLPDWLEDNLSEGIREWWALKPGAPQPKANQQHQDNARGLVLP | 428 |
| acid) | GYKYLGPGNGLDKGEPVNAADAAALEHDKAYDQQLKAGDNPYLKYNHAD | |
| | AEFQERLKEDTSFGGNLGRAVFQAKKRLLEPLGLVEEAAKTAPGKKRPVE | |
| | QSPQEPDSSAGIGKSGAQPAKKRLNFGQTGDTESVPDPQPIGEPPAAPSG | |
| | VGSLTMASGGGAPVADNNEGADGVGSSSGNWHCDSQWLGDRVITTSTR | |
| | TWALPTYNNHLYKQISNSTSGGSSNDNAYFGYSTPWGYFDFNRFHCHFS | |
| | PRDWQRLINNNWGFRPKRLNFKLFNIQVKEVTDNNGVKTIANNLTSTVQVF | |
| | TDSDYQLPYVLGSAHEGCLPPFPADVFMIPQYGYLTLNDGSQAVGRSSFY | |
| | CLEYFPSQMLRTGNNFQFSYEFENVPFHSSYAHSQSLDRLMNPLIDQYLY | |
| | YLSKTINGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGSLIFG | |
| | KQGTGRDNVDADKVMITNEEEIKTTNPVATEAYGQVATNHQSAYAQAQTG | |
| | AVQNQGILPGMVWQDRDVYLQGPIWAKIPHTDGNFHPSPLMGGFGMKHP | |
| | PPQILIKNTPVPADPPTAFNKDKLNSFITQYSTGQVSVEIEWELQKENSKRW | |
| | NPEIQYTSNYYKSNNVEFAVNTEGVYSEPRPIGTRYLTRNL | |
| V134 (amino | MAADGYLPDWLEDNLSEGIREWWALKPGAPQPKANQQHQDNARGLVLP | 429 |
| acid) | GYKYLGPGNGLDKGEPVNAADAAALEHDKAYDQQLKAGDNPYLKYNHAD | |
| | AEFQERLKEDTSFGGNLGRAVFQAKKRLLEPLGLVEEAAKTAPGKKRPVE | |
| | QSPQEPDSSAGIGKSGAQPAKKRLNFGQTGDTESVPDPQPIGEPPAAPSG | |
| | VGSLTMASGGGAPVADNNEGADGVGSSSGNWHCDSQWLGDRVITTSTR | |
| | TWALPTYNNHLYKQISNSTSGGSSNDNAYFGYSTPWGYFDFNRFHCHFS | |
| | PRDWQRLINNNWGFRPKRLNFKLFNIQVKEVTDNNGVKTIANNLTSTVQVF | |
| | TDSDYQLPYVLGSAHEGCLPPFPADVFMIPQYGYLTLNDGSQAVGRSSFY | |
| | CLEYFPSQMLRTGNNFQFSYEFENVPFHSSYAHSQSLDRLMNPLIDQYLY | |
| | YLSKTINGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGSLIFG | |
| | KQGTGRDNVDADKVMITNEEEIKTTNPVATESYGQVATNHQSAYAQAQTG | |
| | AVQSQGILPGMVWQDRDVYLQGPIWAKIPHTDGNFHPSPLMGGFGMKHP | |
| | PPQILIKNTPVPADPPTAFNKDKLNSFITQYSTGQVSVEIEWELQKENSKRW | |
| | NPEIQYTSNYYKSNNVEFAVNTEGVYSEPRPIGTRYLTRNL | |
| V135 (amino | MAADGYLPDWLEDNLSEGIREWWALKPGAPQPKANQQHQDNARGLVLP | 430 |
| acid) | GYKYLGPGNGLDKGEPVNAADAAALEHDKAYDQQLKAGDNPYLKYNHAD | |
| | AEFQERLKEDTSFGGNLGRAVFQAKKRLLEPLGLVEEAAKTAPGKKRPVE | |
| | QSPQEPDSSAGIGKSGAQPAKKRLNFGQTGDTESVPDPQPIGEPPAAPSG | |
| | VGSLTMASGGGAPVADNNEGADGVGSSSGNWHCDSQWLGDRVITTSTR | |
| | TWALPTYNNHLYKQISNSTSGGSSNDNAYFGYSTPWGYFDFNRFHCHFS | |
| | PRDWQRLINNNWGFRPKRLNFKLFNIQVKEVTDNNGVKTIANNLTSTVQVF | |
| | TDSDYQLPYVLGSAHEGCLPPFPADVFMIPQYGYLTLNDGSQAVGRSSFY | |
| | CLEYFPSQMLRTGNNFQFSYEFENVPFHSSYAHSQSLDRLMNPLIDQYLY | |
| | YLSKTINGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGSLIFG | |
| | KQGTGRDNVDADKVMITNEEEIKTTNPVATESYGQVATNHQSAQAQAQTG | |
| | ALQSQGILPGMVWQDRDVYLQGPIWAKIPHTDGNFHPSPLMGGFGMKHP | |
| | PPQILIKNTPVPADPPTAFNKDKLNSFITQYSTGQVSVEIEWELQKENSKRW | |
| | NPEIQYTSNYYKSNNVEFAVNTEGVYSEPRPIGTRYLTRNL | |
| V136 (amino | MAADGYLPDWLEDNLSEGIREWWALKPGAPQPKANQQHQDNARGLVLP | 431 |
| acid) | GYKYLGPGNGLDKGEPVNAADAAALEHDKAYDQQLKAGDNPYLKYNHAD | |
| | AEFQERLKEDTSFGGNLGRAVFQAKKRLLEPLGLVEEAAKTAPGKKRPVE | |
| | QSPQEPDSSAGIGKSGAQPAKKRLNFGQTGDTESVPDPQPIGEPPAAPSG | |
| | VGSLTMASGGGAPVADNNEGADGVGSSSGNWHCDSQWLGDRVITTSTR | |
| | TWALPTYNNHLYKQISNSTSGGSSNDNAYFGYSTPWGYFDFNRFHCHFS | |
| | PRDWQRLINNNWGFRPKRLNFKLFNIQVKEVTDNNGVKTIANNLTSTVQVF | |
| | TDSDYQLPYVLGSAHEGCLPPFPADVFMIPQYGYLTLNDGSQAVGRSSFY | |
| | CLEYFPSQMLRTGNNFQFSYEFENVPFHSSYAHSQSLDRLMNPLIDQYLY | |
| | YLSKTINGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGSLIFG | |
| | KQGTGRDNVDADKVMITNEEEIKTTNPVATESYGQVATNKQSAYAQAQTG | |
| | AVQNQGILPGMVWQDRDVYLQGPIWAKIPHTDGNFHPSPLMGGFGMKHP | |
| | PPQILIKNTPVPADPPTAFNKDKLNSFITQYSTGQVSVEIEWELQKENSKRW | |
| | NPEIQYTSNYYKSNNVEFAVNTEGVYSEPRPIGTRYLTRNL | |
| V137 (amino | MAADGYLPDWLEDNLSEGIREWWALKPGAPQPKANQQHQDNARGLVLP | 432 |
| acid) | GYKYLGPGNGLDKGEPVNAADAAALEHDKAYDQQLKAGDNPYLKYNHAD | |
| | AEFQERLKEDTSFGGNLGRAVFQAKKRLLEPLGLVEEAAKTAPGKKRPVE | |
| | QSPQEPDSSAGIGKSGAQPAKKRLNFGQTGDTESVPDPQPIGEPPAAPSG | |
| | VGSLTMASGGGAPVADNNEGADGVGSSSGNWHCDSQWLGDRVITTSTR | |
| | TWALPTYNNHLYKQISNSTSGGSSNDNAYFGYSTPWGYFDFNRFHCHFS | |
| | PRDWQRLINNNWGFRPKRLNFKLFNIQVKEVTDNNGVKTIANNLTSTVQVF | |
| | TDSDYQLPYVLGSAHEGCLPPFPADVFMIPQYGYLTLNDGSQAVGRSSFY | |
| | CLEYFPSQMLRTGNNFQFSYEFENVPFHSSYAHSQSLDRLMNPLIDQYLY | |
| | YLSKTINGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGSLIFG | |
| | KQGTGHDNVDADKVMITNEEEIKTTNPVATESYGQVATNHQSAYAQAQTG | |
| | AVQNQGILPGMVWQDRDVYLQGPIWAKIPHTDGNFHPSPLMGGFGMKHP | |
| | PPQILIKNTPVPADPPTAFNKDKLNSFITQYSTGQVSVEIEWELQKENSKRW | |
| | NPEIQYTSNYYKSNNVEFAVNTEGVYSEPRPIGTRYLTRNL | |
| V138 (amino | MAADGYLPDWLEDNLSEGIREWWALKPGAPQPKANQQHQDNARGLVLP | 433 |
| acid) | GYKYLGPGNGLDKGEPVNAADAAALEHDKAYDQQLKAGDNPYLKYNHAD | |
| | AEFQERLKEDTSFGGNLGRAVFQAKKRLLEPLGLVEEAAKTAPGKKRPVE | |
| | QSPQEPDSSAGIGKSGAQPAKKRLNFGQTGDTESVPDPQPIGEPPAAPSG | |
| | VGSLTMASGGGAPVADNNEGADGVGSSSGNWHCDSQWLGDRVITTSTR | |
| | TWALPTYNNHLYKQISNSTSGGSSNDNAYFGYSTPWGYFDFNRFHCHFS | |
| | PRDWQRLINNNWGFRPKRLNFKLFNIQVKEVTDNNGVKTIANNLTSTVQVF | |
| | TDSDYQLPYVLGSAHEGCLPPFPADVFMIPQYGYLTLNDGSQAVGRSSFY | |
| | CLEYFPSQMLRTGNNFQFSYEFENVPFHSSYAHSQSLDRLMNPLIDQYLY | |
| | YLSKTINGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGSLIFG | |
| | KQGTGRDNVDADKVMITNEEEIKTTNPVATESYGQVATNHQSAYAQAQTG | |
| | AVQNQGVLPGMVWQDRDVYLQGPIWAKIPHTDGNFHPSPLMGGFGMKH | |
| | PPPQILIKNTPVPADPPTAFNKDKLNSFITQYSTGQVSVEIEWELQKENSKR | |
| | WNPEIQYTSNYYKSNNVEFAVNTEGVYSEPRPIGTRYLTRNL | |
| V139 (amino | MAADGYLPDWLEDNLSEGIREWWALKPGAPQPKANQQHQDNARGLVLP | 434 |
| acid) | GYKYLGPGNGLDKGEPVNAADAAALEHDKAYDQQLKAGDNPYLKYNHAD | |
| | AEFQERLKEDTSFGGNLGRAVFQAKKRLLEPLGLVEEAAKTAPGKKRPVE | |
| | QSPQEPDSSAGIGKSGAQPAKKRLNFGQTGDTESVPDPQPIGEPPAAPSG | |
| | VGSLTMASGGGAPVADNNEGADGVGSSSGNWHCDSQWLGDRVITTSTR | |
| | TWALPTYNNHLYKQISNSTSGGSSNDNAYFGYSTPWGYFDFNRFHCHFS | |
| | PRDWQRLINNNWGFRPKRLNFKLFNIQVKEVTDNNGVKTIANNLTSTVQVF | |
| | TDSDYQLPYVLGSAHEGCLPPFPADVFMIPQYGYLTLNDGSQAVGRSSFY | |
| | CLEYFPSQMLRTGNNFQFSYEFENVPFHSSYAHSQSLDRLMNPLIDQYLY | |
| | YLSKTINGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGSLIFG | |
| | KQGTGRDNVDADKVMITNEEEIKTTNPVATESYGQVATNHQSAYAQAQTG | |
| | ALQNQGILPGMVWQDRDVYLQGPIWAKIPHTDGNFHPSPLMGGFGMKHP | |
| | PPQILIKNTPVPADPPTAFNKDKLNSFITQYSTGQVSVEIEWELQKENSKRW | |
| | NPEIQYTSNYYKSNNVEFAVNTEGVYSEPRPIGTRYLTRNL | |
| V140 (amino | MAADGYLPDWLEDNLSEGIREWWALKPGAPQPKANQQHQDNARGLVLP | 435 |
| acid) | GYKYLGPGNGLDKGEPVNAADAAALEHDKAYDQQLKAGDNPYLKYNHAD | |
| | AEFQERLKEDTSFGGNLGRAVFQAKKRLLEPLGLVEEAAKTAPGKKRPVE | |
| | QSPQEPDSSAGIGKSGAQPAKKRLNFGQTGDTESVPDPQPIGEPPAAPSG | |
| | VGSLTMASGGGAPVADNNEGADGVGSSSGNWHCDSQWLGDRVITTSTR | |
| | TWALPTYNNHLYKQISNSTSGGSSNDNAYFGYSTPWGYFDFNRFHCHFS | |
| | PRDWQRLINNNWGFRPKRLNFKLFNIQVKEVTDNNGVKTIANNLTSTVQVF | |
| | TDSDYQLPYVLGSAHEGCLPPFPADVFMIPQYGYLTLNDGSQAVGRSSFY | |
| | CLEYFPSQMLRTGNNFQFSYEFENVPFHSSYAHSQSLDRLMNPLIDQYLY | |
| | YLSKTINGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGSLIFG | |
| | KQGTGRDNVDADKVMITNEEEIKTTNPVATESYGQVATNHQSAQAQAQTG | |
| | AVQNQGVLPGMVWQDRDVYLQGPIWAKIPHTDGNFHPSPLMGGFGMKH | |
| | PPPQILIKNTPVPADPPTAFNKDKLNSFITQYSTGQVSVEIEWELQKENSKR | |
| | WNPEIQYTSNYYKSNNVEFAVNTEGVYSEPRPIGTRYLTRNL | |
| V141 (amino | MAADGYLPDWLEDNLSEGIREWWALKPGAPQPKANQQHQDNARGLVLP | 436 |
| acid) | GYKYLGPGNGLDKGEPVNAADAAALEHDKAYDQQLKAGDNPYLKYNHAD | |
| | AEFQERLKEDTSFGGNLGRAVFQAKKRLLEPLGLVEEAAKTAPGKKRPVE | |
| | QSPQEPDSSAGIGKSGAQPAKKRLNFGQTGDTESVPDPQPIGEPPAAPSG | |
| | VGSLTMASGGGAPVADNNEGADGVGSSSGNWHCDSQWLGDRVITTSTR | |
| | TWALPTYNNHLYKQISNSTSGGSSNDNAYFGYSTPWGYFDFNRFHCHFS | |
| | PRDWQRLINNNWGFRPKRLNFKLFNIQVKEVTDNNGVKTIANNLTSTVQVF | |
| | TDSDYQLPYVLGSAHEGCLPPFPADVFMIPQYGYLTLNDGSQAVGRSSFY | |
| | CLEYFPSQMLRTGNNFQFSYEFENVPFHSSYAHSQSLDRLMNPLIDQYLY | |
| | YLSKTINGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGSLIFG | |
| | KQGTGRDNVDADKVMITNEEEIKTTNPVATESYGQVATNHQSAYAQAQTG | |
| | AVQNQGILPGMVWQDRDVYLQGPIWAKIPHTDGNFHPSPLMGGFGMKHP | |
| | PPQILIKNTPVPADPPTAFNKDKLNSFITQYSTGQVSVEIEWELQKENSKRW | |
| | NPEIQYTSNYYKSNNVEFAVNTEGVYSEPRPIGTRYLTRNL | |
| V142 (amino | MAADGYLPDWLEDNLSEGIREWWALKPGAPQPKANQQHQDNARGLVLP | 437 |
| acid) | GYKYLGPGNGLDKGEPVNAADAAALEHDKAYDQQLKAGDNPYLKYNHAD | |
| | AEFQERLKEDTSFGGNLGRAVFQAKKRLLEPLGLVEEAAKTAPGKKRPVE | |
| | QSPQEPDSSAGIGKSGAQPAKKRLNFGQTGDTESVPDPQPIGEPPAAPSG | |
| | VGSLTMASGGGAPVADNNEGADGVGSSSGNWHCDSQWLGDRVITTSTR | |
| | TWALPTYNNHLYKQISNSTSGGSSNDNAYFGYSTPWGYFDFNRFHCHFS | |
| | PRDWQRLINNNWGFRPKRLNFKLFNIQVKEVTDNNGVKTIANNLTSTVQVF | |
| | TDSDYQLPYVLGSAHEGCLPPFPADVFMIPQYGYLTLNDGSQAVGRSSFY | |
| | CLEYFPSQMLRTGNNFQFSYEFENVPFHSSYAHSQSLDRLMNPLIDQYLY | |
| | YLSKTINGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGSLIFG | |
| | KQGTGRDNVDADKVMITNEEEIKTTNPVATESYGTVATNHQSAQAQAQTG | |
| | AVQNQGILPGMVWQDRDVYLQGPIWAKIPHTDGNFHPSPLMGGFGMKHP | |
| | PPQILIKNTPVPADPPTAFNKDKLNSFITQYSTGQVSVEIEWELQKENSKRW | |
| | NPEIQYTSNYYKSNNVEFAVNTEGVYSEPRPIGTRYLTRNL | |
| V143 (amino | MAADGYLPDWLEDNLSEGIREWWALKPGAPQPKANQQHQDNARGLVLP | 438 |
| acid) | GYKYLGPGNGLDKGEPVNAADAAALEHDKAYDQQLKAGDNPYLKYNHAD | |
| | AEFQERLKEDTSFGGNLGRAVFQAKKRLLEPLGLVEEAAKTAPGKKRPVE | |
| | QSPQEPDSSAGIGKSGAQPAKKRLNFGQTGDTESVPDPQPIGEPPAAPSG | |
| | VGSLTMASGGGAPVADNNEGADGVGSSSGNWHCDSQWLGDRVITTSTR | |
| | TWALPTYNNHLYKQISNSTSGGSSNDNAYFGYSTPWGYFDFNRFHCHFS | |
| | PRDWQRLINNNWGFRPKRLNFKLFNIQVKEVTDNNGVKTIANNLTSTVQVF | |
| | TDSDYQLPYVLGSAHEGCLPPFPADVFMIPQYGYLTLNDGSQAVGRSSFY | |
| | CLEYFPSQMLRTGNNFQFSYEFENVPFHSSYAHSQSLDRLMNPLIDQYLY | |
| | YLSKTINGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGSLIFG | |
| | KQGTGRDNVDADKVMITNEEEIKTTNPVATESYGQVATNHQSAQAQAQTG | |
| | ALQNQGILPGMVWQDRDVYLQGPIWAKIPHTDGNFHPSPLMGGFGMKHP | |
| | PPQILIKNTPVPADPPTAFNKDKLNSFITQYSTGQVSVEIEWELQKENSKRW | |
| | NPEIQYTSNYYKSNNVEFAVNTEGVYSEPRPIGTRYLTRNL | |
| V144 (amino | MAADGYLPDWLEDNLSEGIREWWALKPGAPQPKANQQHQDNARGLVLP | 439 |
| acid) | GYKYLGPGNGLDKGEPVNAADAAALEHDKAYDQQLKAGDNPYLKYNHAD | |
| | AEFQERLKEDTSFGGNLGRAVFQAKKRLLEPLGLVEEAAKTAPGKKRPVE | |
| | QSPQEPDSSAGIGKSGAQPAKKRLNFGQTGDTESVPDPQPIGEPPAAPSG | |
| | VGSLTMASGGGAPVADNNEGADGVGSSSGNWHCDSQWLGDRVITTSTR | |
| | TWALPTYNNHLYKQISNSTSGGSSNDNAYFGYSTPWGYFDFNRFHCHFS | |
| | PRDWQRLINNNWGFRPKRLNFKLFNIQVKEVTDNNGVKTIANNLTSTVQVF | |
| | TDSDYQLPYVLGSAHEGCLPPFPADVFMIPQYGYLTLNDGSQAVGRSSFY | |
| | CLEYFPSQMLRTGNNFQFSYEFENVPFHSSYAHSQSLDRLMNPLIDQYLY | |
| | YLSKTINGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGSLIFG | |
| | KQGTGRDNVDADKVMITNEEEIKTTNPVATESYGQVATNHQSAQAQAQTG | |
| | WVQNQGVLPGMVWQDRDVYLQGPIWAKIPHTDGNFHPSPLMGGFGMKH | |
| | PPPQILIKNTPVPADPPTAFNKDKLNSFITQYSTGQVSVEIEWELQKENSKR | |
| | WNPEIQYTSNYYKSNNVEFAVNTEGVYSEPRPIGTRYLTRNL | |
| V145 (amino | MAADGYLPDWLEDNLSEGIREWWALKPGAPQPKANQQHQDNARGLVLP | 440 |
| acid) | GYKYLGPGNGLDKGEPVNAADAAALEHDKAYDQQLKAGDNPYLKYNHAD | |
| | AEFQERLKEDTSFGGNLGRAVFQAKKRLLEPLGLVEEAAKTAPGKKRPVE | |
| | QSPQEPDSSAGIGKSGAQPAKKRLNFGQTGDTESVPDPQPIGEPPAAPSG | |
| | VGSLTMASGGGAPVADNNEGADGVGSSSGNWHCDSQWLGDRVITTSTR | |
| | TWALPTYNNHLYKQISNSTSGGSSNDNAYFGYSTPWGYFDFNRFHCHFS | |
| | PRDWQRLINNNWGFRPKRLNFKLFNIQVKEVTDNNGVKTIANNLTSTVQVF | |
| | TDSDYQLPYVLGSAHEGCLPPFPADVFMIPQYGYLTLNDGSQAVGRSSFY | |
| | CLEYFPSQMLRTGNNFQFSYEFENVPFHSSYAHSQSLDRLMNPLIDQYLY | |
| | YLSKTINGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGSLIFG | |
| | KQGTGRDNVDADKVMITNEEEIKTTNPVATESYGQVATNHQSAQAQAQTG | |
| | WVQSQGILPGMVWQDRDVYLQGPIWAKIPHTDGNFHPSPLMGGFGMKHP | |
| | PPQILIKNTPVPADPPTAFNKDKLNSFITQYSTGQVSVEIEWELQKENSKRW | |
| | NPEIQYTSNYYKSNNVEFAVNTEGVYSEPRPIGTRYLTRNL | |
| V146 (amino | MAADGYLPDWLEDNLSEGIREWWALKPGAPQPKANQQHQDNARGLVLP | 441 |
| acid) | GYKYLGPGNGLDKGEPVNAADAAALEHDKAYDQQLKAGDNPYLKYNHAD | |
| | AEFQERLKEDTSFGGNLGRAVFQAKKRLLEPLGLVEEAAKTAPGKKRPVE | |
| | QSPQEPDSSAGIGKSGAQPAKKRLNFGQTGDTESVPDPQPIGEPPAAPSG | |
| | VGSLTMASGGGAPVADNNEGADGVGSSSGNWHCDSQWLGDRVITTSTR | |
| | TWALPTYNNHLYKQISNSTSGGSSNDNAYFGYSTPWGYFDFNRFHCHFS | |
| | PRDWQRLINNNWGFRPKRLNFKLFNIQVKEVTDNNGVKTIANNLTSTVQVF | |
| | TDSDYQLPYVLGSAHEGCLPPFPADVFMIPQYGYLTLNDGSQAVGRSSFY | |
| | CLEYFPSQMLRTGNNFQFSYEFENVPFHSSYAHSQSLDRLMNPLIDQYLY | |
| | YLSKTINGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGSLIFG | |
| | KQGTGRDNVDADKVMITNEEEIKTTNPVATESYGQVATNHQSAQAQAQTG | |
| | WLQNQGILPGMVWQDRDVYLQGPIWAKIPHTDGNFHPSPLMGGFGMKHP | |
| | PPQILIKNTPVPADPPTAFNKDKLNSFITQYSTGQVSVEIEWELQKENSKRW | |
| | NPEIQYTSNYYKSNNVEFAVNTEGVYSEPRPIGTRYLTRNL | |
| V147 (amino | MAADGYLPDWLEDNLSEGIREWWALKPGAPQPKANQQHQDNARGLVLP | 442 |
| acid) | GYKYLGPGNGLDKGEPVNAADAAALEHDKAYDQQLKAGDNPYLKYNHAD | |
| | AEFQERLKEDTSFGGNLGRAVFQAKKRLLEPLGLVEEAAKTAPGKKRPVE | |
| | QSPQEPDSSAGIGKSGAQPAKKRLNFGQTGDTESVPDPQPIGEPPAAPSG | |
| | VGSLTMASGGGAPVADNNEGADGVGSSSGNWHCDSQWLGDRVITTSTR | |
| | TWALPTYNNHLYKQISNSTSGGSSNDNAYFGYSTPWGYFDFNRFHCHFS | |
| | PRDWQRLINNNWGFRPKRLNFKLFNIQVKEVTDNNGVKTIANNLTSTVQVF | |
| | TDSDYQLPYVLGSAHEGCLPPFPADVFMIPQYGYLTLNDGSQAVGRSSFY | |
| | CLEYFPSQMLRTGNNFQFSYEFENVPFHSSYAHSQSLDRLMNPLIDQYLY | |
| | YLSKTINGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGSLIFG | |
| | KQGTGRDNVDADKVMITNEEEIKTTNPVATESYGQVATNHQSAYAQAQTG | |
| | WVQNQGILPGMVWQDRDVYLQGPIWAKIPHTDGNFHPSPLMGGFGMKH | |
| | PPPQILIKNTPVPADPPTAFNKDKLNSFITQYSTGQVSVEIEWELQKENSKR | |
| | WNPEIQYTSNYYKSNNVEFAVNTEGVYSEPRPIGTRYLTRNL | |
| V148 (amino | MAADGYLPDWLEDNLSEGIREWWALKPGAPQPKANQQHQDNARGLVLP | 443 |
| acid) | GYKYLGPGNGLDKGEPVNAADAAALEHDKAYDQQLKAGDNPYLKYNHAD | |
| | AEFQERLKEDTSFGGNLGRAVFQAKKRLLEPLGLVEEAAKTAPGKKRPVE | |
| | QSPQEPDSSAGIGKSGAQPAKKRLNFGQTGDTESVPDPQPIGEPPAAPSG | |
| | VGSLTMASGGGAPVADNNEGADGVGSSSGNWHCDSQWLGDRVITTSTR | |
| | TWALPTYNNHLYKQISNSTSGGSSNDNAYFGYSTPWGYFDFNRFHCHFS | |
| | PRDWQRLINNNWGFRPKRLNFKLFNIQVKEVTDNNGVKTIANNLTSTVQVF | |
| | TDSDYQLPYVLGSAHEGCLPPFPADVFMIPQYGYLTLNDGSQAVGRSSFY | |
| | CLEYFPSQMLRTGNNFQFSYEFENVPFHSSYAHSQSLDRLMNPLIDQYLY | |
| | YLSKTINGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGSLIFG | |
| | KQGTGRDNVDADKVMITNEEEIKTTNPVATESYGQVATNHQSAQAQAQTG | |
| | AVQNQGILPGMVWQDRDVYLQGPIWAKIPHTDGNFHPSPLMGGFGMKHP | |
| | PPQILIKNTPVPADPPTAFNKDKLNSFITQYSTGQVSVEIEWELQKENSKRW | |
| | NPEIQYTSNYYKSNNVEFAVNTEGVYSEPRPIGTRYLTRNL | |
| V149 (amino | MAADGYLPDWLEDNLSEGIREWWALKPGAPQPKANQQHQDNARGLVLP | 444 |
| acid) | GYKYLGPGNGLDKGEPVNAADAAALEHDKAYDQQLKAGDNPYLKYNHAD | |
| | AEFQERLKEDTSFGGNLGRAVFQAKKRLLEPLGLVEEAAKTAPGKKRPVE | |
| | QSPQEPDSSAGIGKSGAQPAKKRLNFGQTGDTESVPDPQPIGEPPAAPSG | |
| | VGSLTMASGGGAPVADNNEGADGVGSSSGNWHCDSQWLGDRVITTSTR | |
| | TWALPTYNNHLYKQISNSTSGGSSNDNAYFGYSTPWGYFDFNRFHCHFS | |
| | PRDWQRLINNNWGFRPKRLNFKLFNIQVKEVTDNNGVKTIANNLTSTVQVF | |
| | TDSDYQLPYVLGSAHEGCLPPFPADVFMIPQYGYLTLNDGSQAVGRSSFY | |
| | CLEYFPSQMLRTGNNFQFSYEFENVPFHSSYAHSQSLDRLMNPLIDQYLY | |
| | YLSKTINGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGSLIFG | |
| | KQGTGHDNVDADKVMITNEEEIKTTNPVATESYGQVATNHQSAQAQAQTG | |
| | WVQNQGILPGMVWQDRDVYLQGPIWAKIPHTDGNFHPSPLMGGFGMKH | |
| | PPPQILIKNTPVPADPPTAFNKDKLNSFITQYSTGQVSVEIEWELQKENSKR | |
| | WNPEIQYTSNYYKSNNVEFAVNTEGVYSEPRPIGTRYLTRNL | |
| V150 (amino | MAADGYLPDWLEDNLSEGIREWWALKPGAPQPKANQQHQDNARGLVLP | 445 |
| acid) | GYKYLGPGNGLDKGEPVNAADAAALEHDKAYDQQLKAGDNPYLKYNHAD | |
| | AEFQERLKEDTSFGGNLGRAVFQAKKRLLEPLGLVEEAAKTAPGKKRPVE | |
| | QSPQEPDSSAGIGKSGAQPAKKRLNFGQTGDTESVPDPQPIGEPPAAPSG | |
| | VGSLTMASGGGAPVADNNEGADGVGSSSGNWHCDSQWLGDRVITTSTR | |
| | TWALPTYNNHLYKQISNSTSGGSSNDNAYFGYSTPWGYFDFNRFHCHFS | |
| | PRDWQRLINNNWGFRPKRLNFKLFNIQVKEVTDNNGVKTIANNLTSTVQVF | |
| | TDSDYQLPYVLGSAHEGCLPPFPADVFMIPQYGYLTLNDGSQAVGRSSFY | |
| | CLEYFPSQMLRTGNNFQFSYEFENVPFHSSYAHSQSLDRLMNPLIDQYLY | |
| | YLSKTINGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGSLIFG | |
| | KQGTGRDNVDADKVMITNEEEIKTTNPVATESYGTVATNHQSAQAQAQTG | |
| | WVQNQGILPGMVWQDRDVYLQGPIWAKIPHTDGNFHPSPLMGGFGMKH | |
| | PPPQILIKNTPVPADPPTAFNKDKLNSFITQYSTGQVSVEIEWELQKENSKR | |
| | WNPEIQYTSNYYKSNNVEFAVNTEGVYSEPRPIGTRYLTRNL | |

### 10. CITATION OF REFERENCES

All publications, patent applications, patents, and other publications and references (e.g., sequence database reference numbers) cited herein are incorporated by reference in their entirety. For example, all GenBank, Uniprot, Unigene, and Entrez sequences referred to herein, e.g., in any Table herein, are incorporated by reference. When one gene or protein references a plurality of sequence accession numbers, all of the sequence variants are encompassed.

1-72. (canceled)

73. A capsid polypeptide comprising an amino acid sequence having at least 95% sequence identity to SEQ ID NO:7 or a VP2 or VP3 portion thereof and comprising:
(a) a histidine at a position corresponding to R550 of the VP1 capsid polypeptide of SEQ ID NO:7;
(b) an alanine at a position corresponding to S576 of the VP1 capsid polypeptide of SEQ ID NO:7;
(c) a threonine at a position corresponding to Q579 of the VP1 capsid polypeptide of SEQ ID NO:7;
(d) a lysine at a position corresponding to H584 of the VP1 capsid polypeptide of SEQ ID NO:7;
(e) a tyrosine at a position corresponding to Q588 of the VP1 capsid polypeptide of SEQ ID NO:7;
(f) an alanine at a position corresponding to W595 of the VP1 capsid polypeptide of SEQ ID NO:7;
(g) a leucine at a position corresponding to V596 of the VP1 capsid polypeptide of SEQ ID NO:7;
(h) a serine at a position corresponding to N598 of the VP1 capsid polypeptide of SEQ ID NO:7; and
(i) a valine at a position corresponding to 1601 of the VP1 capsid polypeptide of SEQ ID NO:7.

74. The capsid polypeptide of claim 73, wherein the amino acid sequence has at least 96% sequence identity to SEQ ID NO:7.

75. The capsid polypeptide of claim 73, wherein the amino acid sequence has at least 97% sequence identity to SEQ ID NO:7.

76. The capsid polypeptide of claim 73, wherein the amino acid sequence has at least 98% sequence identity to SEQ ID NO:7.

77. The capsid polypeptide of claim 73, which comprises the amino acid sequence of SEQ ID NO:12 or a VP2 or VP3 portion thereof.

78. The capsid polypeptide of claim 73, whose sequence has an edit distance of 12 or lower to a VP1 capsid polypeptide of SEQ ID NO:7 or the VP2 or VP3 portion thereof.

79. The capsid polypeptide of claim 73, whose sequence has an edit distance of 11 or lower to a VP1 capsid polypeptide of SEQ ID NO:7 or the VP2 or VP3 portion thereof.

80. The capsid polypeptide of claim 73, whose sequence has an edit distance of 10 or lower to a VP1 capsid polypeptide of SEQ ID NO:7 or the VP2 or VP3 portion thereof.

81. The capsid polypeptide of claim 73, which is a VP1 capsid polypeptide.

82. The capsid polypeptide of claim 73, which is a VP2 capsid polypeptide.

83. The capsid polypeptide of claim 73, which is a VP3 capsid polypeptide.

84. A nucleic acid molecule comprising a nucleotide sequence encoding the capsid polypeptide of claim 73.

85. A virus particle comprising the capsid polypeptide of claim 73.

86. The virus particle of claim 85, further comprising a nucleic acid comprising a payload and a promoter.

87. A virus particle comprising the capsid polypeptide of claim 77.

88. The virus particle of claim 87, further comprising a nucleic acid comprising a payload and a promoter.

89. A host cell comprising a nucleic acid encoding the capsid polypeptide of claim 73.

90. A method of producing a virus particle comprising a capsid polypeptide, the method comprising introducing the nucleic acid molecule of claim 84 into a cell and harvesting virus particles therefrom.

91. A method of producing a virus particle comprising a capsid polypeptide, the method comprising culturing a cell engineered to express the capsid polypeptide of claim 73 and harvesting virus particles therefrom.

92. A method of delivering a payload to a cell, the method comprising contacting the cell with a virus particle comprising the capsid polypeptide of claim 73 and the payload.

93. A method of delivering a payload to a subject, the method comprising administering to the subject a virus particle comprising the capsid polypeptide of claim 73 and the payload.

94. A method of delivering a payload to the CNS of a subject, the method comprising administering to the subject the virus particle of claim 85.

95. A method of delivering a payload to the muscle of a subject, the method comprising administering to the subject the virus particle of claim 85.

96. A method of treating a disease or condition in a subject, the method comprising administering to the subject in an amount effective to treat the disease or condition a virus particle comprising the capsid polypeptide of claim 73 and a heterologous nucleic acid sequence encoding a therapeutic product suitable for treating the disease or condition.

97. The method of claim 96, wherein the disease or condition is a disease or condition of the CNS.

98. The method of claim 96, wherein the disease or condition is a muscle disease or condition.
